SEQUENCE LISTING (365 sequences)

SEQ ID NO: 1 - DNA, 6068 bp, synthetic construct
  source 1..6068: mol_type = other DNA
ggagagaattgatcagaactcctgtcttgtctccggtctttgtgtctcccattttcctcccttctaggtgcttcggggtccctcgtgtagtgtcccgcgggtcgggacaactggcgcccaacgtggggcctgaagtctcctagaagacgagacgcctgagttcgtccggtctaaggagctgcagcatatttcttctttgatcaccataagactacccaacttgtgggagatctgtacaggtaagcggacgactccttcaaaaaaatgggacatatatttgtgttctagacttatgtatagtctacaggcttcccctcagacacttagactagggttcccctaacctgttgtcccagtctccctttttatctgctctcagctcactttgggttttagtcgttcaccaacgagacagttttctaggtgtttgggaccgtttgagcgagattttgcctgcttactttgagctccaatcgtccacccagaggatttcccgaccggttgagtcccgactggctttcgcctgagggtcgttaccagccgcgtcgcctctcgggatccgtgttggcggattataccaaccgattgctcacgtaagggctttttctcctctcaccccaacacccccgtggctccggccgggtgagtcccaaaagacattcgtctgcgggtcgttaccatccgtgccgtctcgtttgggtccatgttggtggattgtaccaaccgactgcctatgtgaggagagtctttattccttatcataatgggacaagaggttagtgttcatgacatgtttatctcaggactaaaagagtccttacaaataaggagagttaaagtcaagaaaaaagatttagttatcttttttaatttcttaaaagatgtttgcccttggctccctcaggaaggaaccatagaccacaaaagatggaaaaggatcggagatgcccttaatgacttttataaaacttttggccctaaaaagaatccccatcactgctttcacttattggaattgcattattgagctacttatggtacatcgctacacccctgacatcgaccgagtgatacaagaaggaaacacatttttacaaaacgcttcccgcccctcctcctccttacaggtcccctcttctaagtcctctcacgattcagattctatttctatttcaatgcctcctgaagatcctgagaccaccaaaaaagatcctagtaagccttatatcctcccctaccacctaattgtcctgatcttaatgtaaattctagcccacctgaggacgatcagttaagccctgaggacgaggctgatttagaggaagctgccgctaaatatcataatcctgtctggcagtttctggcctctaatcaattgccccctccctataatccccaaatgcctttagctcctatccacgatcctgatcaaactctcctctcccaccaagtccaacaattacaaagaactgttcaactcaaaaaacaacatctaactctccttaaacaacttcaacaattagatttacaactctcctctgctgctactcaaaaaattcccccccctttccataaatcctacaaaaacatttcccatctcaaataaaaaaaaccctattaatcttttccccgttattgaattcccccccaataaaaactgaaggaggcagtgcagatagtgataaagaccccgacagagacaatatagaaccccgcaagacactataaacgccttgacttaaaaaccacaaaagaactcaaaaaagcggtggacgaatatggccccacggccccctttacactctcaattttacaatccctagatgacctctggttaaccacccatgattggcactatttggcccatgccaccctatcggggggcgattatgttctctggaaatctgagttttctgaggcctgtaaagaaactgcacaccgcaacgcagaagcgggaggcgagtgcactgattcgacctatgataagttcaggggctttaagccctacgatacaaatgaagctcaactacaatatccatctggccttttttctcaaatttcaccttgccgctactaaggcatggaaaaaacttctccctaaggggccggccacaactcaactcactagtattagacagaggccagaggaaccttatgctgacttcatcagtcgcctaaccaatgccactgaaagactccttggtagcacagaaactgatagtgattttttcaaacaattagcttttgaaaataccaattctgcctgtcaggcagccatctgccctagaaaaaaggattcactctctgattacattcgcctatgcactgatatttggtcctggtcaccaaatgggcctcgctatcggggcagctttaaaagattcattacttaatctgtctaaaggcaaaaacaattgtttttcatgtggccagcccggacatttcgccaaacaatgcccaacccctcgccagaacaccattaggccaacccactcccacacccatattgcccccgcgagtatgtcccagatgcaagagagacaaacattgggccaatcaatgtagatcaaaaatagatgcccacaacaatcctctcctgccccagcaggggaaacttcctgaggggccagccccaggcccctacaggagaatccaaaccttggggcgactcggtttgctcatccacaacaaaactttgtcccatctcaagtctcctccgagcaacccctggcagtgctggactggacctcagtcccctcctccaaatcaatattaactccctgacatgggacctcagatactacctacgggtgtcaccggacccctaccaaccaacacttttggtctaaaattggaagaggtagttcgagcctacaaggcctatatatttaccctggtgttatagataatgattttacgggagaaatacagattgtagcctcctccacttcctctctcatttctatacaaccgggacagagaatagctcaactactccttctcccactccagaccacccataaatctgccaacaatgagcctagaaacaacaaaaattttagatcctcagatgcttattggattcaaaatctctcccccaataagcccatgctagatttaaaacttgatggaaaaaccttttaaaggccttatcgacactggtgctgatgcaaccattattagacaaaaagactggccgctttcttggccccttttctgacacacttactcacctacaaggcataggacaaacaactaaccccagacaaagtgccaagttcctaacatggctagataaagaaaataactctggcacagtacaaccttacgttgtacccaaccctcccagtaaatctgtggggccgtgacatattatcccaaatgggagtaatcatgttcagccccaattccaagataaccatccagatgttaaaacaagggtttctcccaggtcagggattagaaaaacaaggacagggaattaaaaaacccctgtctactgcttcagtgcctgccttcgattaggcttaggacattttcactagtggcctctgaccaacctgcaccccatgctgaccctatatcctggaaaggacaactcgcccatatgggtggatcagtggccactaaattcagaaaaactaaatgctgccaatcagttagtgcagaaacaattggcggcagggcatctagagcccagtaactcccccctggaacacacctatctttgtcgtaaaaagaaatctggaaattggagacttctccaagaccatagggaagtcaataaaacaatgataattatgggcgcccttcaaccaggcctacctaccccctggagctattccctcggggatccttaaaaatcattattgatctcaaagactgcttcttcactatccctctacaccctcaagatagacaatgtttttgctttcagcatacctataactaatttccaagggcccatgcagagatttcagtggaaggtcttacctcaggggcatggccaacagcccgacactgtcaaatatttgtttgctctggccatcgatcccattcgaactcagtggccctctctttatattattcattatatggatgatatcttaatagctggcaagaatgggtctgtacttcctctccccaatataaacaagaaaaacctcagccttgtcccgctaaatgctctactatttaccctattattcatagttcttgttacaatacctataaaacatgtacagaaaagataactcctcttattatacggctgtcatgacaagcactggtcccgctgtccctcattctgactggtctaacacccctgctgcggttggcatttggctcccataaacccgcaccctgcgcggcatctaatatgttagaaaaaaatatttgctgggcagatcgaatcccctataccatatgtttctgacggcggggggtccagccgatctccaatccaatgaaaaacgcattaaaaaatttgctaaatacaaaagacccttaaccctaaatttacctatcaccctttggcccaccctaaaaaaccggggtcacgtggacattgatcctcagacttttgacattcttagttctacccacaagttattgctttctgttaattcatcctacgccagagactgctggctgtgtttactacaaggtacccctttaccattagctataccctatccctttgtcacctctgactaccaataattcatacaacatagctctcccctttttttagtccaaccccttggctttaacaataccccgtgcatcctctctcccattcaaaacaatactacagaggttatatttaggaagcctctcctttacaaattgctcctccttcattaatgtatcctctcctatgtgtacacccaatggatcggtatatatttgtggaaataattattggcctacacctatttaccacaaaactggacaggagttttgtaccctaggctccctcctcccagatgtatccatcattccaggagatgagccagtccctatcccgactttcgaacatattgcaggacgcactaaacgtgcagtccattttattcccttattagcgggtctagacatcaccagcacacttgccaccggggtccgcggggataggaacattccctagtacaataccataaattatctggacaactcatatcagatgtccaggtactctcagaaactaatccaagatcttcaagatcaggttgattccctagcagaagttgtcctccaaaacaggagggggattagatttacttactgcaaaaaaagggggcatctgtctggccctcggagaaaaatgctgttttttatgctaacaaatctggaattgttcgtgaacagagtcaaaaaaattacaaaaagacttgaaaaaaagaagggacctcctttccaaccctctctggaccggattcaatggacttttaccctacttactaccccctgcttggccccatactcgggtgctttatcctactatcactgggaccacatccctcctcaataaactcatgcgctttctcagacaacaaatagaggccttgcaggccaagcccatacaggtccattacacccgacgggagatgcaagagcgaggagatccctatctcccaataacaggagtcataaaacaggactcctcccctgtgagatgaactggatagccaatgacgggtaagaggacagctctctaagtaacattaaaaaatcaaaaacctgtcgctgtaccaggtttcacagagatggactgtcccaacctaagacaggcacagttccctaggtggctcagagctcttttttataaaacagaaacggggggacctgtagtgggcgggtgcctgtaaggcaccaatcacatgactgagaagcatgagatagaggaagttacttgggtctttagataacacccacattctgtaaggtatgtccagagggcttaagaccatcagcctgcggcaaccctgcttatgttaatgcccctccacccagcacaaaaatgtataataacccatgattgagctgcaataaagagagacttgatc

SEQ ID NO: 2 - DNA, 3350 bp, synthetic construct
  source 1..3350: mol_type = other DNA
ggagacctcgtcgcgcagcggagcggtgcaccagccggtccttcgttactaaaggactcaggtggaggtaggtgtgcgttgggccgctgatactcgagcttgtgtgaccggactgcttttaagaaatagacatttacacacatatataatttaaaaaagcaaacaaacatttcaggatgcattacgtacctttattgcctgtcctgcactctattcagtgttctgttcctttgtcagttttaaaatgttggtcctgactcactgtattgctttcatgactctcagatgggtcgcaacacacattttaaaaaatgctgtaagaatccgggaagtgggtggtaccacgttttgaccgactagtgccccgtgtatacctgcgtcaaacagcacgtaggtgtgaatgagcccaagaccggtctcactgtgtcgttggcagaaaagaatccttggcagtttctgacaaaactaaacaaaaaaggatgaaattcacagaaaatttaagttatagccctgccttagttatgtatctttttgcacaatgactaggactttggtaataacctgtttgttttcaacttgaaaaatgcataatgaatatcgtagtatgtcatcaataaatattcatgtataacatacctttcagtgacagcaaaagtttgcatcctactgatggacatttttaaaagaaaaatatttactgaagtttaacaattacacaaaaagcatatgaaagtgaacaactcaatatatttacacaaagcaagcagacccacgtacctagcacccactgtgagaaccaaaatcattaccagaatcccagagactcctgccaaaggtagtgggacctcccagtcactaccttccaagcgtaataattatcctgatttctaccacggtattagtttcacctatcccttcagaccaggctgtctcccataaaccactgaatttcttttgtcgcaaccacttttctctccctctcctctctcccttcttatccctcttcctcttttctctgtttaggagacctgatttctccatttgcaaaaagtatttttgcccaaccttcgtttcacctggaggtctgtcttcctttgcaaagttactttcttgctttgtacaacaggcaactgtcatctctgtatccttccttatctggaactagaagagagttagagtcgtgtagtcgtggccgagtggttaaggcgatggactagaaatccattggggtctccccgcgcaggttcgaatcctgccgactacggggttctttttcttcccgaaccgcgagtgactcggcaaaacccgtggctgaacttgccgggccagagctccagcgacggggagggaaggttccgcgaggagcatggcccagtttctgtcgctccttctttttaggacagctcttcgtgaattttcctccctatgataaagggctgcggtccctgggtcgcagtctcgggtcagcgagagattccaagggatcagtgggcccagcagccatcctcgttagtatagtggtgagtatccccgcctgtcacgcgggagaccggggttcgattccccgacggggaggcagtatgttttgttttgcactcagtcacctgttttggagttcctggagactctgtggtccctgctaaggacatgaatgctacagagctctgtgtgggtgccacaggttctgtgggtccttccccttgcagctctcggcgaccgcccctgcagggctctggggactgaatggcaggggaccttcctgtcagctcttttcaacttgaccctgccccctgccaggcttgtgccactccccgttctgccgctctctgatcagagaaacacttcagagcgactctaaactaccaaaacctagaggggaacttaggttttaagtgacgcaggacttagaacacttactgagacttagtaagagtgtggttgtctgcacgcgcctcccatttgcagaaagagccactgggggcaatgtgcgagatggcaaaaaaaatccacgtgggtcttcaggccctccttcctcctagaggtcacctgggaatggggaccgcccacaggctcagctggggctctttactccatcctgggcaactgctgcccctaggctcttgcacccaagtgtgtgtaggaaggtggttaagtggtctcggacctgtgggaacaggaggcctccaagttccaggatactgctttcaacaagatctgaagctcctagcagtgtgcttttgagtgtatgttagactttatgaactaaagctttctgaaaggaaaaaaaaaaccactgttataaagccatggcagtcgagacagtgtggcccttactcaggaatggataactaaacggatggaacagaacgcatcctaaacagatccactcatacagccatttggtttaaaacaaaggtgatgccgcaatgcactagggaaagaccgttcttttcaataaattgaaaatcaataaattggtggttcaattggatatcaatatggaaataaatgaattacaacataccccaaactcagtcacacggaagtatatttaaacatcaaagggaaagcaataatgtttctgaaaggtaacaggataatttcttcatgactttggagtatgcaagaatttctaaaacagcacaaaaagcagtcgtcacaaaagataagatatatgtatacattacacttcaccaatattggaaacttttgttcatgactagccaccagtaagcaagtacaaggcaaatgttagagcaggtgtttgtattacatgtacctaataagagactgtgtccctagacagagttctccagagaaacagaaccaataagaggtatgcgtatgtaacaagagatctgttttgaggaattggctcacgccattcatttcaacaatgttttgtggctttcagagtataacttttatacttattttgttaaatttattcctattttatttttgctatgatttttaaatggaagtatttacttttgtcctttttcttttcctgtgaaacattaggaggctgacacctcccagatgcaagtatgaagtgctgaaagatagcagggattaatgtccgctaggagggatactccataaacatgcaaagaaatatagcccacacagggagagtttgaaaaaactgcttcagactcataggataatggcacagataaagtgagaagcatacatacaattgaaatgtgcagtgtttagctggctaggacttgaagatgctgattggaagaaagtgctgatccatgtctttccatgtacaagatgcagctcatggaactcgacccttaaagtggtgcctgtttgttctcagaagcaacaagatagag

SEQ ID NO: 3 - DNA, 7955 bp, synthetic construct
  source 1..7955: mol_type = other DNA
gagaattggagatggcggcggcgcagggaacttcgcaggaaccggcggtttcagaacagcccgctgagctgactgcctccgtgcgggcgagcatcgagcggaagcggcagcgggcactgatgctgcgccaggcccggctggcggcccggccctacccgacgacggaggttgcggctaccggaggttcgggccctggcggcgcctgcccctgccttctcccggcgggccgggcggtgccgcgtcccgtgtgtggcgtctacgcctccggactcccagccccgggctttcctcactgcacctgggcggtccagctgcggtctttagcttgggggtgcagcccccctctccgtctggaggtgcccactagtgcccgtccgcgccgcagctctccctttctgttctcttccgatagcctccaccattcccagagatgatgcttgcagaaaacttttagacctgtaacccatctcagtaatctgcacccgcctcttctttcgtcctcagagggcacattccggatccagcacaatgcttgccacgcgcaaggcaccaagaggagcagagagacagtagccaccgccttcgcggggctcacagagtagcctctgttgtgcttcatatgtttgattctcggagctaacctggaaaattagggcagggtttggtatccgtgttggtgaggtggtcgttgcggacaagaaaaacggggtttgcttaggtccgtctcagtaagtgcacaggctaatcaggactcgaactcgggtcatccgacactgggttcagggcctttccttgccaccagctgcccctgctacacaaagcacctctcctacccttaggaagaaaggctgttattgtctggatttcatcttcctcctttcttagggtagctcttcgctgcgtatctgtcgtgtatgtattaatatgtgtaattctccactgtggtcaaataataatcttccccagggtgcctaaaatatagtttgggtcttcagggctagctctataacgtgaagtacatgtgttcctaaagctaatcccatactgtgtgagtagttgagcacagtttaaagctgtgttatctactatccttttgcaacagtcagagtaaggaagagtgaccagtctgggtctgactgcgtgtcttgatattgatacactgaatctgcaaattccagccacctttaataattctggtcttgtccttattgcttgtgtgtgtgtatgttttaattcctttttcagcttgaggcattctagagtcaggagaaaaagttgttcatttgcattgattaatatttatgattctataaaggattctagatctgtacagacagtccccaacttacagtgatttgacttacggtggtgtgaatgttattcagtagaaaccatactttgaattttgatcttttcctgggatagccatatgtagtactatactcttgggatgctgagccacagctccctgctagccacgtgatcatgtgggtaaacaaccgatactctacagtatagtattaaatgcattttctttttttaatgttgtaaacattaaaatattatagagcagagatgtgtattcaaaaaacacagtcataaacagaaacaaaatgtattggatgaaaaaaagacagtgcgcatttgggaagggtgatagtggaaaactatttaacacatcattaaatgcatttttgacttaaaaaattttctatttatgataggtttctctggatgtaaccccattataaactgaggagcatttgtactaaatgtagaatggatgcaaaatagagtataaactagtattaaacttctggtcatggaaagcaaggtagaatgaatattctgtaagatttcttaggcagttacccaagaagtgaactgtgttgtagtattgcatacaacccgctgtgcttttaagacttaggtaggtactgagatttttatcttcgcagtagttttatttcaatgtactgtacaattttccattttctgtatgtgctctgacatacaccatgaaaaagatggggaagaacttgcttagaatgtggtgctaagaagtggtgctgagggcctggtgaaacagcaaggcatagcagctgagaaaaactggcatgatttagcattgttcaggatcttgctctagtttcagccttgactactttagcttcccctcttcttaattctcattgcactcttggtcattccagttatgtgctacacgattcatgaaatcaatatcattctggtatatttattgatttctatccatccagtagatattcatggaatgtttaactatcagaattacagagataaaacactcagtctaatggatggatatacagccaccacttccggaaccttagaagtttccctaaagccacgttttagtcaatcagcaaccctcagacataactactgttctaaccatttgattagtaatagtatctttttttgaacctcatgtaaatggaatcatacagtgcctggatagttttgctcagcataatatctgccagattcatccatgttgttgcatgttttggtagtttatttatatgctatatagttattttttttgtattataccacaattcttccatttttccttttggtggatgtttgggttgtttgcagtttggagctatcatgaagaaaacttttgtgaacattcttttaaaattttcaattacatttgacacacagtattagtttcaggtgtatatcatagtgattagacatttatacaacttacaagtgatcactctgattaagtcttgtagccatctgacaccatacatagttattataatattattgactatatttcttttcccatgactgtttataattggcaatttgtacttcttaatctcttcaccattttcatccattcccccacccccctcccatctggcagccattcagtttgttctctatatctatgagtttgttttgtttgttcgtttatcttgttttttagattccacatttaagtgaaatcacatggtatttgtctttctctgtttgacatttcacttagtataatatccactaggttcatccatgtcacaaatgacaagattttgttttttatagctgagtaatattccattgtatacatataccacatcttctttgtgtattcgtctgtcagtgaactttggttacttccatatcttggctgttgtaaataatgctgcagtgaacataggggtgtgtatatcttttcgaattagtattttggatttttttcagataaatacccagaagtggaattgctgggtcatatggtaattctatttttaattttttgagggacctccatactgttttccgtagtggctgcaccaatttacaaggtgcttttctctacatccttgccaacacttgttgtttattgatttattgatgatggccattctgacacgtgtgacatgatagctcattgtggttttaatttgcatgtccctgatgattagtgacattgagtattttttcatatgtctattggccatctctgtgtcctctggagaaatgtctgttcaggtcctctgcccattttttaaatcagattgtttcgttttgtgtgttaagttgtatgagttccttatatattttggatattaaacccttattggcatcttctcccattcagcaggttatcattttgttttgctaatggcatccttcactgtgcaaaaactgtttagtttgatgtagtcccatttgtttattttttttcttttgtttcccctgccagaggaaacatattcaaagaaatactactaaaagagatgtaaaagcgtttactgcctatattttcttctaggagttttacggttttgggtcttaaatttaactccttaatccatttttagtttattcttatatgtatacagtgatccagtttcattcttttgcatgtatctgtctatagtttttccaacaccatttactgaagagactgtctttacccaattatatatttttgcctcctgtcatagattaattgaccatgtgggcatgggtttatttctgggttctgttccattgatttatgtgtctgtttttatgtcagtaccatgatgttttgattactatggtctagtagtatagtttgatatcaagtagcatgatacctccagctttgttcttctttatcaagatcgctttagctatctggggtctgttgtggggtctacaaattttagggttacttgttctggttctgtgaaaatgccattggtattttgataggaattgcattgaatctgtagattgatttgggtagtatgaacattttaatgatgttaattctttctattcacaaacatagtatatgcttccatttattagtatcttaactttcattcttcagtgtcttacagttttccaagcacaggtcttttacttccttaaattcattcctaggtattttattctatttaatgcaattttaaatgggattgttttcttaatctctctttctgatagtttgttattggtgtataaaaatgcaaccaatttctgaatattaattttgtgtcctgatactttactgaattcatttattagttctaattgtttttttggtggaatcttaaggttctctctatatagtatcatgtcatctgtgaataatgacaattttacttcttccttttcaatttggatggcttttatttctagtctgactgctgtggctgggacttctagtactatgttgaataaaagtgaaagtggcttgttcctgatcttaaaggaaaagctttcagctcttcactactgagtatgatgttagctgtgggtttgtcctatatggcctttattatgttgaggtattttccctctattcccaatttgctgagagtttttatcataaatagatgttggattttgtcaaatgctttttctgcatctattgatatgatcatatgatttttatctttcattttgtttatatagtttatcacattaattgatttgcaaatattgaaccaaccttgcatgccaggaataaatcccacttaatcatggtgtatgaactttttaatgtactgctgaatttggtttgctaatactttgttgaggatttttgcatctatgttgttcatcagggatgttgggcattttttttttttttttgtattgtctctggttttggtatcaggctaatgctggccttgtaaatgagtttgagagccttccctccttttcagttttttggaatgtttggtaaaatttacctgtgaagtcatttggttcagggcttttgtttgttgggagttttttgattactgattcgattttgttagcagttactggtctgttcagattttctgttactgattcagccttaattttctgctgattcaagccttggaagattgtatgtgtctagcgatttatccatctcttccagtttgtccaatttgtcagcatatagttgttctagtgtttccttatacttctttgtatacctgtggtgtcagttgtcgtatctctttcatttctgattttattttggccctctctcttttcttcttgagtctggctaaaggtttatcaattttgtttatcttttcagagaaccatctcttgcttttgttcatcttttctattgtctttttagactctatttttgtttctactgatctttattatctccttccttttacacactttgggctttcttctttttctagttccttcaggtataaggttagattgtttatttgatattttttttttgtttcttgaggtaggcctgtattgctataaatttcctcttagaactgctttcgctgtgtcccatagattttgggctgtcgtgtttttatttgtctcaaggtattttttgattttctccttaattgcattgttgacccagtcattgtttagtaacatgttatttagccgccatgtgtttgtgtgtgtttcagtttttttcttgtaattgatttctagtttcataccagagaagatgcttggtataatttcaatttactgagacttattttgtggcctaacgtggtctatcctagacagtgttccatgtgcacttgaatatactgccgctttttggtgaaatatcctaaaattatcattcaagtccatctggtcttatgtgtcatttaatgtcactctttccttgttggtgggaatatagtgatattttattatggttttaatttgtattttcccagtgaccagtgatgataactttttcatgtgtttactagctatttggataccctcatttgtgaagtccctattcaggtcttttgcctttttttttttttttcagttgggtaatttgtcttttatttatttataggatttcattacatattctggatgtgaatcctttgtcagatatgcatcttgcaaatagcttctcccagtttgcatcctgtcttttcactctcctaatggtgtcttttgatgaatagaggttcttttaatcaagaccagtttaacaatattttttccccaatggttagtacgttaggccactaagaaagttttagctatctcaagttcatgaagttattctcttgtgttttttattttctggaagcattgtgtttcacattcaagattatgatccattaaaaaatgttttttggtgtatattgcatgaagtagggttaaagttcctttattgaaaagaccattttttcctcactgttttgtagtgtcacttttgtcataaatcccagtgtcatttactgaaaagattattattattattatttttttaaccacagaattgccttggagcatttgctgtaaattaaatgaccaaatatgtgtaggtctatttcaagattctctcctattccattgatctctttgtttgtctttgtgtcagtatcacactgtcttaatttatagtaaatagctttatagtaaatctttaaaacctccaattattacatataaatgtgagaatcagcttgtcagcgcccacctcaaggtccccccccccccgatccctccaactactgaggttttgactgggatcatattggagagataaatttggggaggctgagatctttacagtattgaggcttccaatctgcacatggtatatttctccatttatttaggtctttgatttctcttactggtgttttcagtgtagacgttttatacatctcttcctaggtgttatttcttaattctaattgtagattccaatggatattctacatacataatcatatatttgtgaataaagactgatctattgccagccttgatgcttgttttgatttcttaccatcgtgcactagctggcaccttcagataatgttgaatggaaatgtaatagtggacagtgcttgtcctgtttgatatatattaaatttagtgaaagttcctgtttctacacgagggatcatatgggtttacctcgttcaattattgaccacttttacttattttttgtaggcatggctaatgtaaaagcagccccaaagacaattgacacaggaggaggcttctttctggaagaggaagaagaagaagaacatacaattggaaaagttgttcatcaaccaggacctgttatggaatttgattatgcgatatgtgaagaatgtggtagagacttcatggattcttatcttatgaaccactttgatttggcgacttgtgataactgcagagatgctgatgataaacacaagcttataactaaaacagaagcaaaacaagaataccttctgaatgactgtgatttagaaaaaagagaaccagctcttaagtttattgtgaagaagaatcctcatcattcacaatggggtgatatgaaactctacttaaaattacagattgtgaagcgggctcttgaagtttggggtagtcaggaagcattagaagaagctaaggaagttcgacagaaaaaccgagaaaaaatgaaacagaagaagtttgat

SEQ ID NO: 4 - DNA, 6404 bp, synthetic construct
  source 1..6404: mol_type = other DNA
aagcaaatcctagagctttttgttttttatactattctattgaaacaaagtggaaggtttaaagaggcagcacatatacaagtaggtcagtatcccagtcaataaaagtattgttttattgtcaacaagctgaatctaatgcaccacacacacatatatacacatcatcagatagatacagacttggttaatttgatgagtggagcaaatgagaactagactgctgcatctactgttttctatggaagtggacattgagcaacataaatagctgatcaaagatctataagcactgtcaggaaacaagaattccaggtgttttcatgctgtgacaatgagcaactccaagaagattaatcagaaaaatgcataccaaaaaaaaaaaaaaaaaaaggaagaaaaaaaaaagaaaaatgcattcctactcacaaccataccattttgtcttttgtgaactccgtgtgctgtcttggcggtagtgtgacactggagaaatctgtccagcagcatcctccctgttagataccctcactctttcaacctacaatgaaatatattgtttccactgaaatatcacgagggccatctacacagctttttcacgtttttggcagacctcactccttagtgaactcctggggcagtaacctcttccttctcaaaatcatctggatgaatcctcctgttatttgaaaatcatctcactgagcttcaagggtcctcttgtgaattgtgaccatagcctacctcatatcaacaaaagtttccaatatgaggtgtggaaagaggataaactttattcagctgaacagttggtaaacaggaaaaaccgaaagtgcacaccaagacaaaggggaaggggccttttacagagaaagttagtgccctggttcccatttggtccatttttatgcaaatgagaaatccaaatcacacagttctgatcagtcagcatcatatgttctgattggttgttgtgaatcagttctgattggtcagtatagatgcaaatgaggatataacgccacagttctgattggatgggactagtctcagtcctttggaagttccatcaggagttccatcaggaagttcctgacaatggttgacttaggcagcagcaggagcacagttcgggaggtggaaatttcagtctgtggcttttccctgaaatgcagagtgtgcgagaggcttgtgtcaggaatggctgttagactctattaagaatttgagctcagttaaccatgaggaatccttcttggcagattatttcttctcaaggttcacacttatgagggagactgcttcagagcttccaatgaaagggcgggtacaagggtggtgattggactactgatatgtctttcagccataaggctcacattgatgctggtagggatcccattgcatctgcagatggatgtgtgctttacgatttgagaattgactctgacccatgagaaaacagagctcgaagactggctgaggagggtacatttgggtcaatgtgacacagagtattaaagttaaggcacactgttgtcaattcatgtattcagagttgctctgtaatgtccacagttttttagttgttcttcctagaacttctttctcaggaagcacttgaaacttcattgtaacagatgaaaccaagaagtcattttaagctctttttttttttttaaactctttttaaaaaggtattttagtgttttgtttcttagttgactaagaacaatggcacatcattatattaaatactaaaattcagtggtcaaattggcttatttgaaatttagaaggtaaagtgaactttggccaaattcctttcaaatgtaaaataatttcattgtgattcactcagcaacactttgagattaatttgggatttggggatcaaaaactatcaagcttttaggttgatggttagaggactctagaactataattattaatttccttggttgtgccagacagagttgggcattattgctcagaaatgaataaatcaaagttgttttgcatgagaaactcacaaagttgcatgagggacagagtgggtgttgagtgctagagtgaaggatacagagtgttaagcaagtaaagagaagcaacccagaataaacataatgccagaacacatttctaaaattaggttatgctaaagatgattctaaagaaatatgtgggtgtggcaagcaaaataatggcccctcaaaatgtgctaatcctaatccctgaaatatgttaacatgttactttatacagcaaaatggaccttgtacaaatgattaaattaagactattgagatggggagattgttttgtattatctgtgtggatccattgtaatctcaagggtccttgtaagtgaaagaggtagacaagagaatcatacaaagagatgtgattatggaagcagaggtcagagtaatgtggtctcacatgatgccaagttttggaactggatgagtgagtgccattcaataaaggagggtcaggtattatttgttaattcttgacatccatttgctttattctgacagcagctctgtgtttcatttgaggttctgtccctctttcccccactctcagcccgtgggaggtacccatgagccctgcgatgatgtgaaacggctaaacagagcagttcattgcatctctctggctaacgtatttggttcagtgttggacatgtgaccttagccgttctaatctgagtgactgtcaaaactttggtggaaatactaggaaaatagtaaaaacagaagctgcacagttcttttctgcctggttagaatctggaagcatgcagtttagggagatggtggtagtcatttgtggtcacaaatgaccagcattctgaaggtgaaattaaaaaaaaaaaagagaaatgagaaggaactagcaaaacagaaatggcgcatgatcagtgagacttggagcttctgcatccaacgagtcttatcctggaaccaaaaggttattttgagttttttgtttttgttttttctacacaatttgattttgactttctcttacttgcaatcaaactaatctgaagagagtacagaagaaagggcaggcatggatgtttaaatttaaagacatccacgtggattatgctgtaaggaaatggaaaaatggatttaatgatcagaaagtagtgtatatagaagatgtttatttgggatttatcagctcatagatgggagaaagccgggcatattgatcatattgagtgagactagaaggggtttaaggtcagaagttgaagaataccaatgtttaatagtcaggcacagtacaagaaaacttctaaaagacagggagaaatcattgccagagactaaacctaaatttgtcagttttcaaaagtgtagtgtagagattaaataaagagaagacactttaaggaaatttattaaaatgtgaagcagtgctgtgtttttgtctttggatattgggaatatgaatgattttttctcttttcacctaattttctgtatcacttctgaaataaacaatacgttttgttggggtggcctaatggctcagttggttagactgtgagctctcaacaacaaggttgctggttcaattcccgcatgggatggtgggctgcgccccctgcaactaaagattgaaaaacggcgactggacttggagctgagctgtgccctccacacctagattgaagggcaatgacttggagctgatgggccctggagaaacacactgttcccctatatagcacaataaaaaaatttaaataaaatactcataataagtcaacatagaacattgactgtattgaaaatcttgaaatgtttgtcaaaatatggggtcttaaaattaagttcgagaacttgccaccttgcgtttacattggcagcactgtacaaacagctcgataaggtttcataaccttggtatataaatctcacagctgtgtccgtgtggacatgtggcggtgttgctgaatggcattcattattgttgttgtgtgtttttgtgttgcatcgcaagaatgtctgagcttgaattagaacaatgaacaaacattaaatttcttggtaaacctggcaagagtggaagtgaaatcagggacgtgttagtccaagtctatgaggataatgccaagaataaaatggcagtgtactagtggagtaaacgttttttccgaggggagagaacgtgcaactgataaagagaggtcagggcatccaataacgagtagaactgatgaaaaaaattgcaaaaattcatcaaatgatccatcaaagttattggctgactctgagaagcatagtagtccaaggtaaaatcaatagagaaagacaaaatctgaactgaaaatcttggcatgaggaagatgtgtgcaaaaatggtcccgaagtagctcaccggtgaacaaaaacaaaagagagtccaagtttgtcaagaccttttggagaggcaacatgacattttaggccatgttgtcactggtgatgaaacatgggtgtaccaatatgatcctataacagaatgtcaaagtacaaaatggaagtcagccaattctccacgaagaaaaaagttccatcagtccaaatcaagggtccaaacgatgttgctgaccttttttgatatcagagggattattcattatgaatttgtaccaactggacaaacagttaaccaagtttactatttagaagtgcagaaaaggctgcgtgaaaaacttcagacgaaaatggcctgaacgtttctccaacaattcatggattttgcatcatgacaatacaccggctcacacagtctgtgagggagtttttaaccagcaaacaaataaccgtattggaacaccctccctactcacttcacctggcccccaatgccttctctctttacctgatgataaaggaaatattgaaaggaaaacattttgatgacattcaggacatcaagggtaacacgacgagagctctgatgaccattccaggaaaagagttccaaaattgctttgaagggtggactaggcgctggcatcagtgcacagcttcccaaggggagtactttgaaggtgaccacagtgatattcaccaatgagatatgcattactttttctagaatgaattcacgaatgtaattgtcagacctcgtatactataagacaagaatcgtaacctccagtgcttatggagacaaagaaggtgaccaaagtaagtgaagaacccaggtggggacagtagcaaactagagaacacatgtctgatctaaaaggcacagcacagtaagtgatcaagaaggaccaggtttgattctttagagaagcttgacatccacattctacgtgagtctccaaaattgtcagcgttgatcaatacatggaggcaaattaaacatatccaggagacacatttagtctatagggcacttgggattttatatttgctgtttccaaatggttgtgtataatgtgaatatttgtatgtaaaatctttcctttctttggtatcctacgttttatccaaaaattgggccgcagcttgcaataaagacagcttgtcatttagactcattttacccacttcaggaatttttcaaaacttattcacaccacagtccatttgcatttatttttcacagattgttaatcaaatactcaattcctgcataggaccgctgattctaaattattgaaacagttccgttctgttttggtacgaactccaggttcttgatgttttgatgtttaaacctaccctcctgattatgccagggctgtaggaattaaacagacatattgagacagtctatcgcacagcttcaactaaaaggaaggttcatgatttcttactgctgcaggaaaagcatgctggtggtaaacatttattgatctaacgacctgagcgtgaacagagatgcaaaactctttcttcaagggtcggattctacttattagtagactacccatcagcaaatgtctaaagagtctctgagcgccagtgaatgactgatggcaaaaaggaaacaggtgtacttctgtaggccagcagataccgccaatgatatccctttcacttctcgagcccactggtaaagacagttcaagtcagcctaagcgtgttgcaaaggagagagatgaagtaagtacccctcactaactgtaccttttctagaggtttcttacgcttttgaaatctgtgaagtgatacattacacttatacattcagtacttttgaaacaagggttgtatcagaaactcggggaactatttctaaatacacaatgtccaggccttattagattgactcagtcaaaaaccttcagggaggagtccaggcctgtaaaggtttgtaaagttcctcaagtgactgtgagtcgccagcacaactctagctgagaaatactgcagtag

SEQ ID NO: 5 - DNA, 4860 bp, synthetic construct
  source 1..4860: mol_type = other DNA
ttccctcctccacttacacctggaatggttggatgggtccagtgacatagaaggtgtggtggctggcaaaattctgccatactttggggttacatgtatatagatgttaactactatacagatgtgccaggcattgttcactatgtattacatattgaattttcacaataatgttaagaggcaggtacaattaatacctccactttcagatgagaaaattaaggcagagaggttacataatgtgcccaaggtaccacaccttgataaacagcagctgggattctcacccatccagtcagcttcagaatctgtgcacttaactactagatgctatatagaattaatgccaaaactctcaaaatcagagtcatgagagaaaagccaaagccatcatgccaatatttgttaggttaggttaggctatgttaggttcgttttattttttattcccctaatttcctaatcttctacatttaggggaagagatgtgcttctatattcatgaatgtttatgaatgaacatcgtatgggaccattataactggaccctaaggagatatgttcttgacataattcattatcaatgatcagcattctctttgggttgattggccatgtctttatcatctccacgtcctatagaactgttcttatgaagaatatagtcaggacacacacacacatacacacacgcgcgcgcgcgatggggactcttaactagctcacccccaccaaaaagcctcatctagaagcacagagttatcatgaaatactctgggggagggggcatcatgggggtggcaattcaaagagaaatgagaaaaatcacaagatgtttaaatcaatggggatagcgctggaattttccatcctgaagattttttccagggctaaacctctgactgagttttgtttctttaacaaaggaggtggtggtggtggtagattaccttattttcaaaaacgttctttgtaaacatccaaaattatttccatgaaaattgtttctcttacatgtgacctcaattgtactcagctgaccctgtgactacttggagttgtggtggaacaaagtgcaacagtttcctcctggaagtctttcattttcattgtatgaggtgtgataaaaaaaatacagtgaatgtttaaataaaaaatttattacagtaaaagacacattaccattaattctcctcaaaatactcccccttgctttgaacacatgtatccctttgtttttgccactttctgaagcagttctggaagtcctcttttatgagtgtctttaattgtactgtagtggctgctttgatgtcctgaatcaattcaaaaagtttaccttttgtggtcattttttctttagggaagagccagcagtcgcacggtgccagatctggttaataaggcggatgaggacacaccataatgtctttagttgacagaaattgctgtataccagaagcgatgttggagcattgtcatgatagaggatgatttacagcacactgtaaaacacaccttctctcaagtgtatctcacacccaactgactgcaccaaacaagttgaaacttgtcacacatcattactaaggtttgacatgcagcttcttgtattgaatatccctgcctttccattggatggcacttagcagcaacgttcactgtattgtttaatcacacctcgtacttattctgatggagaaatttttgtcagttgagcacactttcctctctcatccttttattttctgtgtctagctttagtttgggatgagggaggacaaagtactattattatgaaattacagtggctctggaggcctctcaaatcctgactatgacacagaaaattctgaaataattcacagcaggagtactataggacttggtcagctttgcattgaacataactccacatctatattggctctgcttttgcttctcaatattaaatgctcaaatatgtcagtgctaggcactattatttatatccctctgaaacatgtttctattcaaggatgcagcattcagaagactcagtccagcgagtgacagaaaaagacttcccttggattatctatgagattgtaatagcttatctgcatatctgctcactgaatactgcctcgatcattcatatatctggctcacaatgggtaatcaataaatgtgtgatgaatggtctacaattcccagattgcagccctaacttgctcatgatggcttccagtagttttctatcaaagccacatgtggtcagtgtgcaggatgaggagtcgagcccttaaaactcaactctagaagacctactgaagcagttattacaacatgctacaatacacaaagaacaagacttgtacatcagaaacaggttgtctgaaaaagttttctattgggggaatgaagcaaattgagcctaagttttctggacaaaaagaaaaggctgatttactcagtttaagtctaagaccaaagaataagtctgagaaaaacaagatgttacctgatcttatatgcactctattattatttttgctttgcatgtcccttgtaatagtgattggttttaatgatcatttcatataaaaattaaaaagaagtacattttttaacttcctgtcaaaaattctgcctaaggtacttcctcaacacacacacgttagttgctacccctccttcaaggctctgttcatgcccgtctcctccacgaagacttttttgttctacacctagaaaggctctgcctactcaggcagttgttattacctccgatttcctactatcagatctcttcgtattatcttcttatatgactaggtctcatctccccctcaaccacaatctctctgagggctggaatattgtgcacattgccttgcacataataaaggctccagaggtatctgtctaaactggctttatttccttgagactacaagcacttattctgtgccaggcacttttaggttccagggaaaaagaggtacaaaaccagacacaaaccctaccgttatggagcttacctttttaattaaaaggtggaagggatgaacctttttttggtctctctagaaagttgcagcaggagaccataggaaatagtataaaatagttgaaagcactgtggagtgtgagtcaggataccttggtctcatctctaatttgatgtatcttgagcacatttcttaaacattggtcatctgtttccctgtatgccatataggaatcatatggttactgggaaaactgaatcagaaaacagatgcaaatcatgttggagggaactttctcaacctgataaaaagcatctatgaaaaacccacagctaacaccatacttaaaggtgaaagactggaagccttcttccgaagatcagtaacaagacaaggatgtctgctctcaccactgctattcaacattctaccggaagttctagccaggttctaagtaagaaaatgaaataaaaagaatcaagattggaaatgaagaagtactaaaactatctattttcatatgacatgaccttacttagaaaatgctaaagaatccacccccaacccccaccccaacaaaactattaaagctaataagtgaattcagcaagatttcaggatacaaggtcaatacggaaaaaaaaaagttgtatttctataaactaacaatgaacaatctgaaaatgaaattaaaaaacaacaccatttatgatagcattaaaaagaaattaaggaataaatttagcaaagaagtgtaacacttgtacgtggaaaacaacaaaacattgttgaaagaaatcaaagacctaaataaaatttttaaaatcctgcctttgtggattagaacacttaattttgttaaaatagcagtactcctcaatttgaattattcacagcaaatcctacaaaaatcttagctacctttattttcctgcagaaattgacaagctgagtttaaattttacatggaaatgcaaggaacccagaatatccaaaacaatcttgaaaaaaaggaacaaagtgggaagactcatacttcctaatttaaaaactgacggcaaagctacagtaatcaagactatgaggtactggcataaagacagacatataaatcaatggaatagactatgagtccagaataaatccatggtcaattgatttttgataaatgtgccaagacaattcaatggaagaaaataatcttttcaacaaatggtgctaagacaactggatatccacatgcaaaaggatgaattttgaaaccctacctcacaccatatacaaaaattagcttgaaatggatcaaagatatacaaataagtgttacaactataaaacttgaagaaaacataggtgtaaatctccatgaccttggattaagcaatgtcttcttagatacaacatcaaagcacaagcaacaaaagaaaacaattggatttcatcaaaattgaaaacttttgtgagccaaccctcacaaccctcacacggtggctcaggtggttggagcgccatgctggttcgattcccacgtgggccagtgcgctgcatcctctacagctaagactgtgaacaacggctctccctggagctgggctgccacgggctgccgtgggctaccatgtgctgccaggagcggctggtggccagcgtgagtgaccggcagccagcgagaactgacatgaagtgctgtgagtggccgagaggtccaaccagtaaccgactgcctcagctggggggagcgcaaggctcataataccagcatgggccagggagctgtgtcctacatagctagactgagaaacaatagcttacgccggagtggtgggggaggcggaaggggaaaacaacaacaacaacaacaacaaaa

SEQ ID NO: 6 - DNA, 9649 bp, synthetic construct
  source 1..9649: mol_type = other DNA
aaattaagactcacgttagggaaggctgagacaagcagcagaaaccactagataggaacaagaaatgtgaggaaatcaaggcagggagcatgtgaagtggcagggaggggacaatggaagagtgaaacagagcagaggtgacaggcagcagaagagaaagtgattagaagagaaggtggtacattaagctgttggtaataacagagacaagaaatcgcaatagaggaagagtgttgcttctgaaaggaaaaaatctaaattaactaactaaaagcaatctacgatcacaactctacctgttaggagcaaatagcactatatacctacatacctctgtcatcccacatgcattacagtgctgccctggacaaacatgagggtgaataagtccccgctttccctgggaatgtcccagtcttagcacggaaagtcctgtatcccaagaaaacacacacacagtagcagtctaatcaggacagttgttcaccctgattagcattgactcaaaatagcagtgcagtttggggctggtctgtaaagtgtccccttagtggtactcaggattattactgcttcacagtaaccacacacatgctagtaagtgttaagatccggaattgtccccctcagacagacagagaacccgcacaataatgcaagtcacacagcgaggtttattaccagccggctggggtccccgtctctgcccgacgcagcgggtttttaacaaggaccccaaacacttaaagccaaggggttatatagtatttttaaggccttccatagcttctgagagtacaagataaacttacaggacttacatagtttcaaagagtataagatttactacaggacaaggagaccaggagtataagataagctacaggacttctagtcgccatgctgcaactgcccacatcctggaattttataattatgttgtttcaggctaggggctgttaaccctgacctgaagatagcagttctcatgctaacagtctcttacatgctaacagtctcttacatttcccccctgtgtgttgttcataatgaggaatcttgctcatgtacgggccagccgtagaggtcctcacgggtgggcactgtcttatactgttgtcttaggaccatgagctggacagtattaacacgatcttttacaaaattaataagcttgtttaatatgcaagggcccaaggttagaataagcacaagcaagataattgggcccactaaggtagatactaaggtggtgagccaaggagatctgttaaaccaagcctcaaaccagttctcctgagcttccctttcccgttttctcttagctaacccttctctcacctttgccatggattcttttaccacaccagtatggtcagcataaaaacaacattcctcccccagcgcggcacacagtcccccttgttggaggaacaacaaatcaagtcctcttttattctggagtactacctcggatagagaggtgagcgacttctctaaatgactaatcgaggtttccaacctttctatgtcctcatctatggctgcccttagggaggtcattcctgattgttgagtagccagggaagctatgccggtcccagctccggctattcccaaactgaacagggtggcaatggttagtgcggtgatgggctctcttttacttcttgtgtcactatcccaatgcgaatacatactctcctcagggtgatagaggatgcggggcagcactgttactaggacacagaattcattggcggcattaaagaccgaggtagataagcacggggtgaggccagtctttgaacatatccaccatccatcagttctggggattaaccacttagtgtcacttttccaactaggggagctgtctatggaggcacataaactttgtttagcctggggcacctttcctagacaggtcccattgccgctcactagttgcatggttaagccaattttacgatttccccatgaacactgagaagggttcttaccgttagaggcgttgtaagtggcattaagtcctattgcctcatagaacgggggctttatatcatagcacagccaacaggaggttgtgaggttaggactggtggcattaagggtctcgtatacagtgcgcaccagtttccgcaatgagtctttggttggctgcgtagcaggcgtcaaaggagttacagaggtctttggctgggttcccgcagtccctcctgcggtgtttttatcccttgatatacctgggttcttggtagggacgagaggggccagcaccttatttggacccacctgagtgctgatcatttccaccgatagtcgaatagttaggagaccgccggggtggggacctatccatgcccaacggcctatatctaattggaacccccaagttaatcctgataaccaaccacgctctcttcgcgccacgtcttggttaaactggacacgtacctggggcacccggttgtgggggtccctaaaggagaatttaactagatccctgttcccaacgtcccactgtcggggcccgtcataggaggtgacacaactccaactaccacaatagtagcggtctgggccgccacaggttttccaattgtttcttaggttccctgggcaggcccaaaacccttgtgcactatgtccttgagaggtgtcaattacagcccgcttggacctgactgagtagtcatactgccgtccacgtttagtgccgaaaatgtcacgcaggtcgaaaaacaagtctggccaccacgtattgatgggggcagtatgtgtggtgctattaagggttgtttgggtctgtccatctgttagggtccatgttagcttatggggttggtgtgggttgatccccgcgtggctcttctcccagatattgagcagagttagggctagcagccattccatcgttagctgaggcagggggcttgacgcttccccgaggtcgggagagctgcagcttcagagggttatcagggtgtcgccgtacgatccactccttagcttgcgtcttctccagctggctggctcggcgcacgtgagagtgatggacccaaggcccaatgccgtcaacctttaaggcagtgggggtaaccagaataaccacataaggacctttccatctctcctccagtgtccgggacctgtgtctccttacccatacccaatcccctggaacgatgccatgttccgggtttggggcgtccttaatttcatacagggaactcactaggggccatatctcatgttggaccccttgtagggcctttaaactggccagataacttggggccacattggggtcatgatctggtagagtacgaacaataatggggggtggtgccccatacagaatttcgaaaggtgtcaaaccatgtacatatggtgagttccggacccggaagatggcatagggtaagagggtcacccagtccccgccagtctcgatggctagtttggacaaggtctcctttagagtccgattcattctctctacctgccctgagctctggggattatattcacaatgtaacttccaattgatccctatcgctcgggctagtccttgtaggacgttactgatgaaagctgggccgttatcggagcctaaaacctcaggaaccccatatctgggaataatttcttctagtaatgccttagcaaccacttgggcagtctcccgtttcgtggggaaggcttccacccagcccgaaaatgtgtcaaccattactagcaagtacttatacccacacctcccaggctttacctcagtaaaatccacttcccaactccgtcccggcgctcttccccgtaccctcgtacctgtatgttggggtccttttctactgggtctcatagcctggcacccaatgcactgatctacaatctcttgaatctgagccgcttgtcggggaaaccggaggcgggcggactcgagaattgtcagcaacttcttttttcctaagtgggtggcttgatgcaggttggagagaagaaacagtcctagctgtgccggcagtatcaatcttccttctgtatcccgatgccacccctgctgatcagattccgggcagtggtggttctggatccatcgcaggtcttctggagtgtagtcaggtcgcgggggcaggcgagggagctcaggtgtgggcagggtgagtgctaaagctgatgaagctactgccactgccttggcggcttcatccgctcgccggtttcctttagcttccggggtctgggcagactggtgcccagggatgtggacaactgcgactgcccggggcatttgtacagccatcagcagtcttcgtacctcaggaagattgcgcagagtctttccttccgctgtaacaaagcctctttcccggtagatagcgccatgcacatggacagtgccaaaggcgtagcggctatcggtgtagacagtcactcgtctcccttcggcccgttccagcgcttccgccagcgcgatcagttcggccttctgtgctgatgtccccggggaaagcgaggcactccaaatgatgtttcccccttggtctaccaccgctgcgcctgccctccgcacaccatctataacgaagctgcttccatcagtgtaccataccaactcactgttgggtagtgcggtgtcctggaggtcggggcgcacctgggtgacttctgccatgatctcttggcagtcatgcaggggagctctcagatccggggtcggcagcagggtggctggattcagagcggtgggttcagcgaagatgatccggggtgcatctagcaagagtccttggtaatgtgttagtcgggcattagtcatccacctaccagggggatatttcaggaccccctcgatcgcatggggggttactaccttcagatgttgcccaaaagtgagtttatcagcatccttcaccattagggctactgccgcaatgatcctcaagcacgggggccatcctgctgcaactggatctagcttcttggataaataggcaaccgggcgtttccagggccccagacgctgcattagcacccctttcgctattcccctcctctcatcaacaaagagagtgaagggcttcagggggtctggcaatgccagagccggggctcttaggagagcgaccttgagttcatcgtaggccttctgttggtctgacccccaggcccaagggaccttatccttggttgcctcatacagaggttttgctatttcagcataccccaaaatccacagccggcagtagcctgtcgtccctaaaaactcacggacctctcgtgctgaggtcgggactggaagtctaagaatagtctctttcatggcctctgtcagccatctggctcctttttttagtttataccccaggtaggtgactgtttgcctgcatatttgagccttctttgcactggcccgatagcccaactgccccagctcctggaggaggtctccagtggcctgtcggcattcagcttcggagggggctgccagaagcaagtcatctacgtactgcaggagcgtaactgaattatggctctggcgaaacgagtccaaatcctgatttagggcttcattaaacagagttggagagtttttgaagccttgcggtagtctagtccaggtcagctgcccgggggttcccgtattgccatcattccattcgaaagcaaaaatgtgttggctgctgggtgccagggctatgctaaaaaacccatcctttaggtctaaggtagtataccagacatgtgaagggggcaagtgacttagtaaggtataagggttggggaccgtgggatggatgtcttcaaccctcttatttacttccctcaagtcctggactggcctataatcttttccccccggtttcttaacggggagaagtggggtgttccaggcagaatggcaaggtttcagtattccagcttccagtaaacggttaatgtgcggggcaatccctttccgcgcctctgcagacatggggtactggcggatccggataggctgggctgaggctttaagttccaccactactggtgctcggcgggccgcccggcccacacccgctatttttgcccacgcctgagggtatgttttaagccaataatccatatcacggggccattctgtagagggagggttgtaggggttgtcctgcagggcgaacaggcgatgttcatccacaagagacagggtcaaaatgtggaggggctgtccttggccatccaatagcttaatgccatccggctcaaaatggatctgagccctgatcttagtcaggagatcgcgccccaataagggggcagggcattcagggataactaggaaggagtgggtcacttggtggcggcctaagtctacttggcgcctactagtccaccgataagccttggacccagttgccccttgcaccaaactggttttctgagataagggctctgtgggcttattcaaaactgagtactgggctcctgtgtctaccaggaatcctactggcttcccctccacatacgcagttacccaagactcggggaggggatccgagtcccgtctcccctagtcactctccatccccgccagcaggacccgtgcgtcttgccctgtttggccctggcgcttggggcactccctcttccaatgtccatactctttgcagtttgcacactgtcccctatccagtcggggccgcggtctccacggtcgggctggtccggccggactcggtcccaccctgactgtgctttgcacgcctgccaacaagatcttggccatctccctctgctgcctcctgttctccctactctgatgctctctgtcttccttcctgattcgttcctgtaattcctgattttcttttctaattctatcctccctttcttcgggagtctctcgagtgttgaagactctctccgctactttcattaaatcccggatagacatttctcccagtccctcctgtttgtacaatttcttcctaatatctggggcagcctggtttataaaggacataattacagccgactggttttcctctgccagggggtccaacggggtgtactgtctgtaagcatcatagaggcgttctaaaaacagggccgggctttcattatccccttgcattatagcttttaccttggccaaattggtggggcggcgtgccgccgctcggagacctgccataagagtctggcggtagactcggagacgctccctaccttctgcgttcccaaagtcccaatccggtctattcaggggaaagcgctcgtctatcaggttcggcaaggtcgtcggtcttccgttgtcgccggggacattttttctggcctcggtgaggattcgctcgcgctcctcggtggtgaataaggtcttaagaagctgctggcaatcatcccaagtgggactgtgtgtgtgcatgacagactcgaacaggtcagttaggcctttcgggtcctcagaaaaaggagggttttgagccttccagttgtacagatcactgctagaaaaaggccagtactggtatgctcgctccccatctggaccagttcctcctagtgctcgcacagggagaatcggcgccccagcggaggtggaggaggacggttcctcctctggggtctcttcccggcgtctgcgaggcctcaatcccctcgccggcccttgagctggggcaggggaacccgggggagggggagccatcggggaggcggtagagtgaggcagctcggaaggagcggcagcctcaggcgggagcggcgccatcgggctgggcgcgcgccgcggctggagcggcgccaccggcgcgtaaggagggggggtctcttccagatctaggtctatcagggaggggtatatgtctgacccctcctgaaggatgggtccctgggtcggcttctccgggggtttcgggccggccgtgggcactgccgactggcgggagggtcccgaaacggtcaggactttaagggggagggggggatcttctggcttgtcggggataaagggcttaagccaggagggaggactctctactaaggcttgccacatcaaaatatagggatattggtccggatggcgccgattaataatatctcggacctttttaataatgtctagggaaaaagttccctgggggggccagcccacattaaaagtaggccattctgcagagcagaatgtatcaaacttacctttcttcacttccacaccatgattacgagccttggcgcggatttcaggaaagtggttcaggagcagggtcttaggtgttacctgaacctgtcccataattgtcacaaagagaaaccaagaaaaggcaaaagaaaggacaaaagacacagtgccagcaaatacacaacttcgcacaggactcttcaacacccaccggccggtcaaccacaccacatccacaggcgccgtttcaatcacaccagtctcaccacgctcaagatccttacctagggcccgtccaaacggcgtccactgtggacgtcgctgggccaccttctcgtcggggacgtctcccacgacttcaagtaacgaagcctccagggtcgtaacctgcactttccttcccgtgagaattctcaactgggaccgggcagagacctgtttcagtctctccggtcgaggacctgtttcagtcctcccctgtttgggaccgggcagagacctgtttcagtctctccggtcgaggacctgtttcagtcctcccctgtttgggaccgggcagagacctgtttcagtctctccggtcgaggacctgtttcagtcctcccctattggaggtggccaaacctccttccgcggttccctatgtaaacctcggtatcgggagttgtctgttcccctgagggggggcgtcccgggcgagcccccaaatgttaagatccggaattgtccccctcagacagacagagaacccgcacaataatgcaagtcacacagcgaggtttattaccagccggctggggtccccgtctctgcccgacgcagcgggtttttaacaaggaccccaaacacttaaagccaaggggttatatagtattttcaaggccttccatagcttctgagagtacaagataaacttacaggacttacatagtttcaaagagtataagatttactacaggacaaggagaccaggagtataagataagctacaggacttctagtcgccatgctgcaactgcccacatcctggaattttataattatgttgtttcaggctaggggctgttaaccctgacctgaagatagcagttctcatgctaacagtctcttacatgctaacagtctcttacagtaagaagttccaaagcctgtggtggcagtaagtgaatttcttccttttcaatagactatgaaggagggacattgcatttgaactcagtccatgagtcatgatgctctttatgtccattaaaaggattaactttctctctattcactatttctttcacactattgtatagggtaacgtgtttggggagaaaaatcaataaaaatgcttaaaataaaagtttccatgctcataaggtttttatcttccattataggaaaatgaatctatatggaagggtacattttctgatgatgttttgtaagaagcattattctatcaatctattaaaatatattgatgcactttcc

SEQ ID NO: 7 - DNA, 77 bp, synthetic construct
  source 1..77: mol_type = other DNA
tcctcgttagtatagtggtgagtatccccgcctgtcacgcgggagaccggggttcgattccccgacggggaggcagv

SEQ ID NO: 8 - protein, 11 aa, synthetic construct
PRRGIEPRSPA

SEQ ID NO: 9 - DNA, 65 bp, synthetic construct
  source 1..65: mol_type = other DNA
tatagtggtgagtatccccgcctgtcacgcgggagaccggggttcgattccccgacggggaggca

SEQ ID NO: 10 - DNA, 2140 bp, synthetic construct
  source 1..2140: mol_type = other DNA
ttcatccatgtcacaaatgacaagattttgttttttatagctgagtaatattccattgtatacatataccacatcttctttgtgtattcgtctgtcagtgaactttggttacttccatatcttggctgttgtaaataatgctgcagtgaacataggggtgtgtatatcttttcgaattagtattttggatttttttcagataaatacccagaagtggaattgctgggtcatatggtaattctatttttaattttttgagggacctccatactgttttccgtagtggctgcaccaatttacaaggtgcttttctctacatccttgccaacacttgttgtttattgatttattgatgatggccattctgacacgtgtgacatgatagctcattgtggttttaatttgcatgtccctgatgattagtgacattgagtattttttcatatgtctattggccatctctgtgtcctctggagaaatgtctgttcaggtcctctgcccattttttaaatcagattgtttcgttttgtgtgttaagttgtatgagttccttatatattttggatattaaacccttattggcatcttctcccattcagcaggttatcattttgttttgctaatggcatccttcactgtgcaaaaactgtttagtttgatgtagtcccatttgtttattttttttcttttgtttcccctgccagaggaaacatattcaaagaaatactactaaaagagatgtaaaagcgtttactgcctatattttcttctaggagttttacggttttgggtcttaaatttaactccttaatccatttttagtttattcttatatgtatacagtgatccagtttcattcttttgcatgtatctgtctatagtttttccaacaccatttactgaagagactgtctttacccaattatatatttttgcctcctgtcatagattaattgaccatgtgggcatgggtttatttctgggttctgttccattgatttatgtgtctgtttttatgtcagtaccatgatgttttgattactatggtctagtagtatagtttgatatcaagtagcatgatacctccagctttgttcttctttatcaagatcgctttagctatctggggtctgttgtggggtctacaaattttagggttacttgttctggttctgtgaaaatgccattggtattttgataggaattgcattgaatctgtagattgatttgggtagtatgaacattttaatgatgttaattctttctattcacaaacatagtatatgcttccatttattagtatcttaactttcattcttcagtgtcttacagttttccaagcacaggtcttttacttccttaaattcattcctaggtattttattctatttaatgcaattttaaatgggattgttttcttaatctctctttctgatagtttgttattggtgtataaaaatgcaaccaatttctgaatattaattttgtgtcctgatactttactgaattcatttattagttctaattgtttttttggtggaatcttaaggttctctctatatagtatcatgtcatctgtgaataatgacaattttacttcttccttttcaatttggatggcttttatttctagtctgactgctgtggctgggacttctagtactatgttgaataaaagtgaaagtggcttgttcctgatcttaaaggaaaagctttcagctcttcactactgagtatgatgttagctgtgggtttgtcctatatggcctttattatgttgaggtattttccctctattcccaatttgctgagagtttttatcataaatagatgttggattttgtcaaatgctttttctgcatctattgatatgatcatatgatttttatctttcattttgtttatatagtttatcacattaattgatttgcaaatattgaaccaaccttgcatgccaggaataaatcccacttaatcatggtgtatgaactttttaatgtactgctgaatttggtttgctaatactttgttgaggatttttgcatctatgttgttcatcagggatgttgggcattttttttttttttttgtattgtctctggttttggtatcaggctaatgctggccttgtaaatgagtt

SEQ ID NO: 11 - DNA, 2314 bp, synthetic construct
  source 1..2314: mol_type = other DNA
taaatacccagaagtggaattgctgggtcatatggtaattctatttttaattttttgagggacctccatactgttttccgtagtggctgcaccaatttacaaggtgcttttctctacatccttgccaacacttgttgtttattgatttattgatgatggccattctgacacgtgtgacatgatagctcattgtggttttaatttgcatgtccctgatgattagtgacattgagtattttttcatatgtctattggccatctctgtgtcctctggagaaatgtctgttcaggtcctctgcccattttttaaatcagattgtttcgttttgtgtgttaagttgtatgagttccttatatattttggatattaaacccttattggcatcttctcccattcagcaggttatcattttgttttgctaatggcatccttcactgtgcaaaaactgtttagtttgatgtagtcccatttgtttattttttttcttttgtttcccctgccagaggaaacatattcaaagaaatactactaaaagagatgtaaaagcgtttactgcctatattttcttctaggagttttacggttttgggtcttaaatttaactccttaatccatttttagtttattcttatatgtatacagtgatccagtttcattcttttgcatgtatctgtctatagtttttccaacaccatttactgaagagactgtctttacccaattatatatttttgcctcctgtcatagattaattgaccatgtgggcatgggtttatttctgggttctgttccattgatttatgtgtctgtttttatgtcagtaccatgatgttttgattactatggtctagtagtatagtttgatatcaagtagcatgatacctccagctttgttcttctttatcaagatcgctttagctatctggggtctgttgtggggtctacaaattttagggttacttgttctggttctgtgaaaatgccattggtattttgataggaattgcattgaatctgtagattgatttgggtagtatgaacattttaatgatgttaattctttctattcacaaacatagtatatgcttccatttattagtatcttaactttcattcttcagtgtcttacagttttccaagcacaggtcttttacttccttaaattcattcctaggtattttattctatttaatgcaattttaaatgggattgttttcttaatctctctttctgatagtttgttattggtgtataaaaatgcaaccaatttctgaatattaattttgtgtcctgatactttactgaattcatttattagttctaattgtttttttggtggaatcttaaggttctctctatatagtatcatgtcatctgtgaataatgacaattttacttcttccttttcaatttggatggcttttatttctagtctgactgctgtggctgggacttctagtactatgttgaataaaagtgaaagtggcttgttcctgatcttaaaggaaaagctttcagctcttcactactgagtatgatgttagctgtgggtttgtcctatatggcctttattatgttgaggtattttccctctattcccaatttgctgagagtttttatcataaatagatgttggattttgtcaaatgctttttctgcatctattgatatgatcatatgatttttatctttcattttgtttatatagtttatcacattaattgatttgcaaatattgaaccaaccttgcatgccaggaataaatcccacttaatcatggtgtatgaactttttaatgtactgctgaatttggtttgctaatactttgttgaggatttttgcatctatgttgttcatcagggatgttgggcattttttttttttttttgtattgtctctggttttggtatcaggctaatgctggccttgtaaatgagtttgagagccttccctccttttcagttttttggaatgtttggtaaaatttacctgtgaagtcatttggttcagggcttttgtttgttgggagttttttgattactgattcgattttgttagcagttactggtctgttcagattttctgttactgattcagccttaattttctgctgattcaagccttggaagattgtatgtgtctagcgatttatccatctcttccagtttgtccaatttgtcagcatatagttgttctagtgtttccttatacttctttgtatacctgtggtgtcagttgtcgtatctctttcatttctgattttattttggccctctctcttttcttcttgagtctggctaaaggtttatcaat

SEQ ID NO: 12 - DNA, 663 bp, synthetic construct
  source 1..663: mol_type = other DNA
ttttcttctaggagttttacggttttgggtcttaaatttaactccttaatccatttttagtttattcttatatgtatacagtgatccagtttcattcttttgcatgtatctgtctatagtttttccaacaccatttactgaagagactgtctttacccaattatatatttttgcctcctgtcatagattaattgaccatgtgggcatgggtttatttctgggttctgttccattgatttatgtgtctgtttttatgtcagtaccatgatgttttgattactatggtctagtagtatagtttgatatcaagtagcatgatacctccagctttgttcttctttatcaagatcgctttagctatctggggtctgttgtggggtctacaaattttagggttacttgttctggttctgtgaaaatgccattggtattttgataggaattgcattgaatctgtagattgatttgggtagtatgaacattttaatgatgttaattctttctattcacaaacatagtatatgcttccatttattagtatcttaactttcattcttcagtgtcttacagttttccaagcacaggtcttttacttccttaaattcattcctaggtattttattctatttaatgcaattttaaatgggattgttttcttaatct

SEQ ID NO: 13 - DNA, 563 bp, synthetic construct
  source 1..563: mol_type = other DNA
aggttcatccatgtcacaaatgacaagattttgttttttatagctgagtaatattccattgtatacatataccacatcttctttgtgtattcgtctgtcagtgaactttggttacttccatatcttggctgttgtaaataatgctgcagtgaacataggggtgtgtatatcttttcgaattagtattttggatttttttcagataaatacccagaagtggaattgctgggtcatatggtaattctatttttaattttttgagggacctccatactgttttccgtagtggctgcaccaatttacaaggtgcttttctctacatccttgccaacacttgttgtttattgatttattgatgatggccattctgacacgtgtgacatgatagctcattgtggttttaatttgcatgtccctgatgattagtgacattgagtattttttcatatgtctattggccatctctgtgtcctctggagaaatgtctgttcaggtcctctgcccattttttaaatcagattgtttcgttttgtgtgttaagttgtatgagttccttatatatt

SEQ ID NO: 14 - protein, 32 aa, synthetic construct
PTSLMNNIDAKILNKVLANQIQQYIKKFIHHD

SEQ ID NO: 15 - protein, 28 aa, synthetic construct
MTSQVNFTKHSKKLKRREGSQTHLQGQH

SEQ ID NO: 16 - protein, 1525 aa, synthetic construct
MFVVVVVVVFPFRLPHHSGVSYCFSVLCRTQLPGPCWYYEPCAPPSGSRLLVGPLGHSQHFMSVLAGCRSLTLATSRSWQHMVAHGSPWQPSSRESRCSQSLRMQRTGPRGNRTSMALQPPEPPCEGCEGWLTKVFNFDEIQLFSFVACALMLYLRRHCLIQGHGDLHLCFLQVLLHLFVYLSISSFLYMVGRVSKFILLHVDIQLSHHLLKRLFSSIELSWHIYQKSIDHGFILDSSIPLIYMSVFMPVPHSLDYCSFAVSFIRKYESSHFVPFFQDCFGYSGFLAFPCKITQLVNFCRKIKVAKIFVGFAVNNSNGVLLFQNVFSTKAGFKFYLGLFLSTMFCCFPRTSVTLLCIYSLISFCYHKWCCFLISFSDCSLLVYRNTTFFFPYPCILKSCIHLLALIVLLGWGLGVDSLAFSKGHVIKIVLVLLHFQSFFLFHFLTNLARTSGRMLNSSGESRHPCLVTDLRKKASSLSPLSMVLAVGFSMLFIRLRKFPPTFASVFFSFPSNHMIPIWHTGKQMTNVEMCSRYIKLEMRPRYPDSHSTVLSTILYYFLWSPAATFRDQKKVHPFHLLIKKVSSITVGFVSGFVPLFPWNLKVPGTEVLVVSRKSQFRQIPLEPLLCARQCAQYSSPQRDCGGGDETSYKKIIRRDLIVGNRRQLPEAEPFVNKKVFVEETGMNRALKEGQLTCVCGSTLGRIFDRKLKNVLLFNFYMKSLKPITITRDMQSKNNNRVHIRSGNILFFSDLFFGLRLKLSKSAFSFCPENLGSICFIPPIENFFRQPVSDVQVLFFVYCSMLLLQVFSVLRARLLILHTDHMWLKTTGSHHEQVRAAIWELTIHHTFIDYPLARYMNDRGSIQADMQISYYNLIDNPREVFFCHSLDVFMLHPIETCFRGIIIVPSTDIFEHLILRSKSRANIDVELCSMQSPSPIVLLLIISEFSVSSGFERPPEPLFHNNSTLSSLIPNSTQKIKGERKVCSTDKNFSIRISTRCDTIQTLLLSAIQWKGRDIQYKKLHVKPCVTSFNLFGAVSWVDTLERRCVLQCAVNHPLSQCSNIASGIQQFLSTKDIMVCPHPPYPDLAPCDCWLFPKEKMTTKGKLFELIQDIKAATTVQLKTLIKEDFQNCFRKWQKQRDTCVQSKGEYFEENWCVFYCNKFFITFTVFFLSHLIQKKTSRRKLLHFVPPQLQVVTGSAEYNGHMEKQFSWKFWMFTKNVFENKVIYHHHHLLCRNKTQSEVPWKKSSGWKIPALSPLITSCDFSHFSLNCHPHDAPSPRVFHDNSVLLDEAFWWGASESPSRARVCVCVCVSLYSSEQFYRTWRRHGQSTQRECSLIMNYVKNISPGPVIMVPYDVHSTFMNIEAHLFPMKIRKLGEKIKRTHSLTPNKYWHDGFGFSLMTLILRVLALILYSILSAQILKLTGWVRIPAAVYQGVVPWAHYVTSLPFSHLKVEVLIVPASHYCENSICNTTMPGTSVLTSIYMPQSMAEFCQPPHLLCHWTHPTIPGVSGGGXXXXXXXXXXXCX

SEQ ID NO: 17 - protein, 2027 aa, synthetic construct
MYCSISQLELCWRLTVTGTLQTFTGLDSSLKVFDVNLIRPGHCVFRNSSPSFYNPCFKSTECISVMYHFTDFKSVRNLKRYSGVLTSSLSFATRLGLELSLPVGSRSERDIIGGICWPTEVHLFPFCHQSFTGAQRLFRHLLMGSLLISRIRPLKKEFCISVHAQVVRSINVYHQHAFPAAVRNHEPSFLKLCDRLSQYVCLIPTALASGGVTSKHQEPGVRTKTERNCFNNLESAVLCRNVFDQSVKNKCKWTVVISFEKFLKWVKVMTSCLYCKLRPNFWIKRRIPKKGKILHTNIHIIHNHLETANIKSQVPYRLNVSPGYVFASMYSTLTILETHVECGCQASLKNQTWSFLITYCAVPFRSDMCSLVCYCPHLGSSLTLVTFFVSISTGGYDSCLIVYEVQLHSIHSRKSNAYLIGEYHCGHLQSTPLGKLCTDASASTLQSNFGTLFLEWSSELSSCYPCPECHQNVFLSIFPLSSGKERRHWGPGEVSREGVPIRLFVCWLKTPSQTVAGVLSCKIHELLEKRSGHFRLKFFTQPFLHFIVNLVNCLSSWYKFIMNNPSDIKKGQQHRLDPFGLMELFSSWRIGLPFCTLTFCYRIILVHPCFITSDNMANVMLPLQKVLTNLDSLLFLFTGELLRDHFCTHLPHAKIFSSDFVFLYFYLGLLCFSESANNFDGSFDEFLQFFSSVLLVIGCPDLSLSVARSLPSEKTFTPLVHCHFILGIILIDLDHVPDFTSTLARFTKKFNVCSLFFKLRHSCDATQKHTTTIMNAIQQHRHMSTRTQLDLYTKVMKPYRAVCTVLPMTQGGKFSNLILRPHILTNISRFSIQSMFYVDLLVFYLNFFIVLYRGTVCFSRAHQLQVIALQSRCGGHSSAPSPVAVFQSLVAGGAAHHPMRELNQQPCCELTVPTEPLGHPNKTYCLFQKYRKLGEKRKNHSYSQYPKTKTQHCFTFISLKCLLFISLHYTFENQIVSLAMISPCLLEVFLYCALLNIGILQLLTLNPFSHSISICPAFSHLADKSQINIFYIHYFLIIKSIFPFPYSIIHVDVFKFKHPCLPFLLYSLQISLIASKRKSKSNCVEKTKTKNSKPFGSRIRLVGCRSSKSHSCAISVLLVPSHFSFFFLISPSECWSFVTTNDYHHLPKLHASRFPGRKELCSFCFYYFPSISTKVLTVTQIRTAKVTCPTLNQIRPERCNELLCLAVSHHRRAHGYLPRAESGGKRDRTSNETQSCCQNKANGCQELTNNTPSFIEWHSLIQFQNLASCETTLLPLLPSHLFVFSCLPLSLTRTLEITMDPHRYKTISPSQSFNHLYKVHFAVSNMLTYFRDDHILRGHYFACHTHIFLNHLHNLILEMCSGIMFILGCFSLLAHSVSFTLALNTHSVPHATLVSHAKQLFIHFAIMPNSVWHNQGNLFSPLTINLKAFLIPKSQINLKVLLSESQNYFTFERNLAKVHFTFISNKPIPLNFSIYNDVPLFLVNETKHNTFLKRVKKKKSLKLLGFICYNEVSSASERSSRKNNKTVDITEQLIHELTTVCLNFNTLCHIDPNVPSSASLRALFSHGSESILKSSTHPSADAMGSLPASMALWLKDISVVQSPPLYPPFHWKLSSLPHKCEPEEIICQEGFLMVNAQILNRVQPFLTQASRTLCISGKSHRLKFPPPELCSCCCLSQPLSGTSWNSWNFQRTETSPIQSELWRYILICIYTDQSELIHNNQSEHMMLTDQNCVIWISHLHKNGPNGNQGTNFLCKRPLPLCLGVHFRFFLFTNCSAESLSSFHTSYWKLLLIGRLWSQFTRGPLKLSEMIFKQEDSSRFEGRGYCPRSSLRSEVCQKREKAVMALVIFQWKQYISLVERVRVSNREDAAGQISPVSHYRQDSTRSSQKTKWYGCEECIFLFFFFLFFFFFLVCIFLINLLGVAHCHSMKTPGILVSQCLIFDQLFMLLNVHFHRKQMQQSSSHLLHSSNPSLYLSDDVYICVCGALDSACQNNTFIDWDTDLLVYVLPLTFHFVSIEYKKQKALGFA

SEQ ID NO: 18 - protein, 228 aa, synthetic construct
TCTCACCTAGCAGGAAGGSCADMTCTCAGGACCATCCCATACAGCAGGGTGGAGGATTGGTGGATCAGGTACATAGGCCCAATACGTCTGGTCTTCTTCTGCATTGCTGAGGGTCATCAATGTCATCAGCAGGTAGAGGGTCCACATGTCGCACCAATCGTTCTGGCAGCCACCGAGGGCCTTCTGCATCCTGTGGAAAAACACAAACATGCCCTCGGCCCCATATGA

SEQ ID NO: 19 - protein, 75 aa, synthetic construct
SHLAGRXXLRTIPYSRVEDWWIRYIGPIRLVFFCIAEGHQCHQQVEGPHVAPIVLAATEGLLHPVEKHKHALGPI

SEQ ID NO: 20 - DNA, 351 bp, synthetic construct
  source 1..351: mol_type = other DNA
ccttggatgcccatggtaagagtgctgtggagcgcttttggcatccttctgctgcccctcaggctttggtcaaatggaaagacccacttacaggctcttggcaaggcccagatccagtcctcatatggggccgagggcatgtttgtgtttttccacaggatgcagaaggccctcggtggctgccagaacgattggtgcgacatgtggaccctctacctgctgatgacattgatgascadmccctctgcatcctgtggaaaaacacaaacatgccctcggccccatatgaggactggatctgggccttgccaagagcctgtaagtgggtctttccatttgaccaaagcctga

SEQ ID NO: 21 - protein, 116 aa, synthetic construct
PWMPMVRVLWSAFGILLLPLRLWSNGKTHLQALGKAQIQSSYGAEGMFVFFHRMQKALGGCQNDWCDMWTLYLLMTLMXXPSASCGKTQTCPRPHMRTGSGPCQEPVSGSFHLTKA

SEQ ID NO: 22 - DNA, 555 bp, synthetic construct
  source 1..555: mol_type = other DNA
agggtccacatgtcgcaccaatcgttctggcagccaccgagggccttctgcatcctgtggaaaaacacaaacatgccctcggccccatatgaggactggatctgggccttgccaagagcctgtaagtgggtctttccatttgaccscadmaatggaaagacccacttacaggcttttggcaaggcccagatccagtcctcatatggggccgagggcatgtttgtgtttttccacaggatacagaaggccctcggtggctgccagaacgattggtgcgacatgtggaccctctacctgctgatgacattgatgaccctcaacaatgcagaagaagaccagacgtattgggcctatgscadmatcccatacagcagggtggaggattggtggatcaggtacataggcccaatacgtctggtcttcttctgtattgctgagggtcatcaatgtcatcagcaggtagagggtccacatgtcgcaccaatcgttctggcagccaccgagggccttctgtattctgtggaaaaacacaaacatgccctcggccccatatga

SEQ ID NO: 23 - protein, 184 aa, synthetic construct
RVHMSHQSFWQPPRAFCILWKNTNMPSAPYEDWIWALPRACKWVFPFDXXNGKTHLQAFGKAQIQSSYGAEGMFVFFHRIQKALGGCQNDWCDMWTLYLLMTLMTLNNAEEDQTYWAYXXIPYSRVEDWWIRYIGPIRLVFFCIAEGHQCHQQVEGPHVAPIVLAATEGLLYSVEKHKHALGPI

SEQ ID NO: 24 - DNA, 1515 bp, synthetic construct
  source 1..1515: mol_type = other DNA
ccaaagccscadmtcgaatttccagagcctctgaaaagatatcagtggcgagtccttccccaagacatggcaaatagccccaccttgtgtcagaagtttgttagtaaaacaattgataacaccagaaaacagtttccttctgtgtacattattcattatatggatgacattttattggcttgtaagaaagaaggagtattgttagcttgctttgcaaatctgcaaaagaatcttctaacctcgggtcttattattgcacccgaaaaaatacagagaagtgagccttgttcttacttgggatttcagttgtttgctcagtatttcactccacaaaaaaaagagcttagaaaagatcatcttaaatctcttaatgattttcaaaagttgttgggagatattaattggctgcacccttctttgggattaactactggagatcttaaaccactgtttgaaattttaaaaagagattctgatccgacctcccccaggtctcttactgagcctgcacggaaggctctctctaaggttgagaaagccattcagcaacagcatgtttcctttttagattattctaaacctctatatgtgtatattttagataccaaacacacgcccacggcggtgttatggcaagaagggccacttagatggatacacctccacgtggctgctcaaaagaatcttactccttattatgaacttgtggccagtttaattcaggagagtcgcttagaagctcgaaaatattatggaaaggagccagattctattgttatcccttttacaaaaatgcagattcaaggcctgatgcagtttacaaacagttttcctatcgccttggctcattttgcggggactttggataatcattatcctaagcataaattgcttcaattttttcaacatcatgatccaatttttccttcaattgtgtcccatgctcctcttcctgctgtacctaatgtttttactgatggatctagcaatggtgtagctgtctatgcactcaatgaaaaagtcaccaagagagtgcagacacctccagcctcagctcaaattgttgagcttcgagcagttcatatggtattgcttgattttgcttcccagtcttttaatttattctctgacagccattatgtggttcgtgccgtcagaaatttagaaacagtaccttttattagcaccagtaatcctgttattcaggatctgtttcttcagatacaacaagccattcagctgcgctgtaacaaattttatattggccatattagagctcactctaatcttccaggccctttagcctcaggaaatcaaactgcagattctgccacacagctcattgttttaactcaaatagaaaaggcacaaaaggctcttagcttccaccatcaaaacaaccagagcttaagactgcaatatactataactagagaaacagcacgccagatagtaaaacaatgcccagattgttcgcatttacagcctgtgcctcattatggagtcaacccttga

SEQ ID NO: 25 - protein, 504 aa, synthetic construct
PKPXXEFPEPLKRYQWRVLPQDMANSPTLCQKFVSKTIDNTRKQFPSVYIIHYMDDILLACKKEGVLLACFANLQKNLLTSGLIIAPEKIQRSEPCSYLGFQLFAQYFTPQKKELRKDHLKSLNDFQKLLGDINWLHPSLGLTTGDLKPLFEILKRDSDPTSPRSLTEPARKALSKVEKAIQQQHVSFLDYSKPLYVYILDTKHTPTAVLWQEGPLRWIHLHVAAQKNLTPYYELVASLIQESRLEARKYYGKEPDSIVIPFTKMQIQGLMQFTNSFPIALAHFAGTLDNHYPKHKLLQFFQHHDPIFPSIVSHAPLPAVPNVFTDGSSNGVAVYALNEKVTKRVQTPPASAQIVELRAVHMVLLDFASQSFNLFSDSHYVVRAVRNLETVPFISTSNPVIQDLFLQIQQAIQLRCNKFYIGHIRAHSNLPGPLASGNQTADSATQLIVLTQIEKAQKALSFHHQNNQSLRLQYTITRETARQIVKQCPDCSHLQPVPHYGVNP

SEQ ID NO: 26 - DNA, 681 bp, synthetic construct
  source 1..681: mol_type = other DNA
ggcctacgtcctaatgatttatggcaaatggatgtaacacatatacctgaatttggaaaattaaaatatgttcatgtctccatagacacattttctggctttgtcgtggctaccgctcaaactggagaggacacatctcatgttattagacattgtcttgctgcttttgctatgattggaacacctaaaaaacttaaaacagataatggctcaggttataccagcaaaaaattctctttattttgccagcaattctcgatcaatcatgttactggcattccttacaatccccaagggcaagggattgttaaacgcactcatggcacattaaaagtcaatttacagaaaataaaaaagggggagttatatcccctgacgccccataattacctgtctcattctctctttatccaaaattttttgaccttggatgcccatggtaagagtgctgcggagtgcttttggcatccttctactgccactcaggctttggtcaaatggaaagacccacttacgggctcttggcaaggcccagatccagtcctcatatggggccgaggacatgtttgtgtttttccacaggatgcagaaggccctcggtggctgccagaacgattggtgcgacatgtggaccctctacctgctgatgacattgatgacscadmggctttggtcaaataa

SEQ ID NO: 27 - protein, 226 aa, synthetic construct
GLRPNDLWQMDVTHIPEFGKLKYVHVSIDTFSGFVVATAQTGEDTSHVIRHCLAAFAMIGTPKKLKTDNGSGYTSKKFSLFCQQFSINHVTGIPYNPQGQGIVKRTHGTLKVNLQKIKKGELYPLTPHNYLSHSLFIQNFLTLDAHGKSAAECFWHPSTATQALVKWKDPLTGSWQGPDPVLIWGRGHVCVFPQDAEGPRWLPERLVRHVDPLPADDIDDXXALVK

SEQ ID NO: 28 - DNA, 309 bp, synthetic construct
  source 1..309: mol_type = other DNA
tggacacataaaacaacatttgaaaagttttgtaaatcaggcactccctgcagtcaggtgactgatttacaagatgggactagagactggtctaaaaaatctgttaatgtatctgcttgtgttcscadmgggtcatcaatgtcatcagcaggtagagggtccacatatcgcaccaatcgttctggcagccaccgagggccctctgtatcctgtggaaaaacacaaacatgccctcggccccatatgaggactggatctgggccttgccaagagcctgtaagtgggtctttccatttgaccaaagcctga

SEQ ID NO: 29 - protein, 102 aa, synthetic construct
WTHKTTFEKFCKSGTPCSQVTDLQDGTRDWSKKSVNVSACVXXGSSMSSAGRGSTYRTNRSGSHRGPSVSCGKTQTCPRPHMRTGSGPCQEPVSGSFHLTKA

SEQ ID NO: 30 - DNA, 234 bp, synthetic construct
  source 1..234: mol_type = other DNA
ggcgtgagtgtcattgacataatctggaatctcaggaccatcccatacagcagggtggaggattggtggatcaggtacataggcccaatacgtctggtctttttctgcattgttgagggtcatcaatgtcatcagcaggtagagggtccacatgtcgcaccaatcgttttggcagccaccgagggccctctgtatcctgtggaaaaacacaaacatgccctcggccccatatga

SEQ ID NO: 31 - protein, 77 aa, synthetic construct
GVSVIDIIWNLRTIPYSRVEDWWIRYIGPIRLVFFCIVEGHQCHQQVEGPHVAPIVLAATEGPLYPVEKHKHALGPI

SEQ ID NO: 32 - DNA, 297 bp, synthetic construct
  source 1..297: mol_type = other DNA
agggtccatatgtcgcaccaatcgttctggcagccaccgagggccctctgcatcctgtggaaaaacacaaacatgccctcggccccatatgaggactggatctgggccttgccaagagcctgtaagtgggtctttccatttgaccaaagcctgagtggcagtagaaggatgccaaaagcgctctgcagcactcttaccatgggcatccaaggtcaaaaaattttgaataaagagagaatgagacaggtaattatggggcgtcaggggatacaactcccccttttttattttttgtaa

SEQ ID NO: 33 - protein, 98 aa, synthetic construct
RVHMSHQSFWQPPRALCILWKNTNMPSAPYEDWIWALPRACKWVFPFDQSLSGSRRMPKALCSTLTMGIQGQKILNKERMRQVIMGRQGIQLPLFYFL

SEQ ID NO: 34 - DNA, 258 bp, synthetic construct
  source 1..258: mol_type = other DNA
ctttggatgcctatgttaagagtgcagctgaacgtttctggcatccttctgccgtccctgaggctttggtcagaaagaaggatccacttactggatcatggcaaggcccagacccagtcctcatatggggccgagggcatgtttgtgtttttccacaggatgcaaascadmaggagaaacaagaatggtggtggctttatatcgcagataggaaggaacagacattcgtatctatgccatatcatgtctgtacattaa

SEQ ID NO: 35 - protein, 85 aa, synthetic construct
LWMPMLRVQLNVSGILLPSLRLWSERRIHLLDHGKAQTQSSYGAEGMFVFFHRMQXXRRNKNGGGFISQIGRNRHSYLCHIMSVH

SEQ ID NO: 36 - DNA, 399 bp, synthetic construct
  source 1..399: mol_type = other DNA
cattatacccctcaatacctgaacacgtatcttctaagaacaagggccttttacatcagcacaatacaattattatattcaggaagtttaacattgatatggtattattgtctaatatgcaatccgtattcaaatttcctcaaatactccactaatacccgttacagtctttgtcttgtttttaagttcaggatccaatcagggatcacacattgcatttggttgccattcctcgttagcacacttcttggcctttttctttttaaatttttcatgccattgatatttttgaggcgtccaggcaaggtattttgtaaattagcccttaatttgaatttgtctcattggttactcctgattgtattcatcttaaatatttttggcaaaaatacaacatag

SEQ ID NO: 37 - protein, 132 aa, synthetic construct
HYTPQYLNTYLLRTRAFYISTIQLLYSGSLTLIWYYCLICNPYSNFLKYSTNTRYSLCLVFKFRIQSGITHCIWLPFLVSTLLGLFLFKFFMPLIFLRRPGKVFCKLALNLNLSHWLLLIVFILNIFGKNTT

SEQ ID NO: 38 - DNA, 258 bp, synthetic construct
  source 1..258: mol_type = other DNA
ttcagtgctgacactgtctacctggatctgataatatcagatcccacaggtcaagggctcagtcccacaggacggctgtcccccccttcagatgccaatcacaagtcgcaggttgtcacctatatacaccaaatggctataaatcagggtacccgcgactccctccttgggttcagtaatttgccggaatggttcacagaactcaggaaaacacattaccagtttattatgaaagactatgataaaggatatatatga

SEQ ID NO: 39 - protein, 85 aa, synthetic construct
FSADTVYLDLIISDPTGQGLSPTGRLSPPSDANHKSQVVTYIHQMAINQGTRDSLLGFSNLPEWFTELRKTHYQFIMKDYDKGYI

SEQ ID NO: 40 - DNA, 348 bp, synthetic construct
  source 1..348: mol_type = other DNA
aggttagatatagatattttcctattatctcacagcatttatcttagaaataagaacttggttagaatgattgcctttctggtgaagtctattttatttcaacatttctttcattattttattttaaaataccaaattaacatgttgtatgccttaaatttgcacaatgttacatgtcaaatacattttttttttaaacttttacttattttaagtgtgttttcccaggacccatcagctccaagtcaagtagtttcaatcgagttgtggagggcgcagctcacagtggcccatgtggggattgaaccagcaaccttgttgttaagagctcacgctctaaccgactga

SEQ ID NO: 41 - protein, 115 aa, synthetic construct
RLDIDIFLLSHSIYLRNKNLVRMIAFLVKSILFQHFFHYFILKYQINMLYALNLHNVTCQIHFFFKLLLILSVFSQDPSAPSQVVSIELWRAQLTVAHVGIEPATLLLRAHALTD

SEQ ID NO: 42 - DNA, 276 bp, synthetic construct
  source 1..276: mol_type = other DNA
cattttagagtatactctttgtgtatgtatcatttgaagcacactcccattagtgtttaccattttacttgggatttttataaaagtcattctatggtgttaaagagattgtgctgcagtatagtttcactgtgtactgcagtcccaaaggaaagggagccagtaaagacgtgccgctttttttccacaagagtaccatatttcttaacgttggctataaaattttacttcatgagtcccgaagcagcaaaatacctctttgaaagtcacatttga

SEQ ID NO: 43 - protein, 91 aa, synthetic construct
HFRVYSLCMYHLKHTPISVYHFTWDFYKSHSMVLKRLCCSIVSLCTAVPKEREPVKTCRFFSTRVPYFLTLAIKFYFMSPEAAKYLFESHI

SEQ ID NO: 44 - DNA, 228 bp, synthetic construct
  source 1..228: mol_type = other DNA
tgtctacatttaattctttgtagttggaagttcacgaggctaagcccgtgccagaaaatcacccgcagtgggatacagcagtggagggggatgaagaccaggaggacagcgagggctttgaagacagctttgaggaagaggaggaagaagaggaagatgacgactaagcagtactgcaaacggaccacaatactttcacattttcactgttttggaagtgtagaataa

SEQ ID NO: 45 - protein, 75 aa, synthetic construct
CLHLILCSWKFTRLSPCQKITRSGIQQWRGMKTRRTARALKTALRKRRKKRKMTTKQYCKRTTILSHFHCFGSVE

SEQ ID NO: 46 - DNA, 429 bp, synthetic construct
  source 1..429: mol_type = other DNA
ctttggatgcctatgttaagagtgcagctgaacgtttctggcatccttctgcggtccctgaggctttggtcagaaagaaggatccacttactggatcatggcaaggcccagacccagtcctcatatggggccgagggcatgtttgtgtttttccacaggatgcagaaggccctcggtggctgccagaacgattggtgcgacatgtggaccctctacctgctgatgacattgatgaccctcagcaatgcagaagaagaccagacgtattgggcctacgtacttgatccacctattctccaccctgctgtgtgggatggtcctgagattccagactatgtcaatgacactcacgccctaggattgccttctgatggacacataaaacatttggaaagttttgtaaatcaggcactccctgcagtcaggtga

SEQ ID NO: 47 - protein, 142 aa, synthetic construct
LWMPMLRVQLNVSGILLRSLRLWSERRIHLLDHGKAQTQSSYGAEGMFVFFHRMQKALGGCQNDWCDMWTLYLLMTLMTLSNAEEDQTYWAYVLDPPILHPAVWDGPEIPDYVNDTHALGLPSDGHIKHLESFVNQALPAVR

SEQ ID NO: 48 - DNA, 237 bp, synthetic construct
  source 1..237: mol_type = other DNA
tacttaaacaaccatcttttgttatgcttcctgttaatatctctggaccttggtatactaaaagaaatttggcatgatgttaatgtgtctttagatatgtttcagcttcatgagaaaattcaaaatscadmtcatcagcaggtagagggtccacatgtcgcaccaatcgttctggcagccactgagggccttctgcatcctgtggaaaaacacaaacatgccctcggccccatatga

SEQ ID NO: 49 - protein, 78 aa, synthetic construct
YLNNHLLLCFLLISLDLGILKEIWHDVNVSLDMFQLHEKIQNXXHQQVEGPHVAPIVLAATEGLLHPVEKHKHALGPI

SEQ ID NO: 50 - DNA, 498 bp, synthetic construct
  source 1..498: mol_type = other DNA
ccaaagcctgaggggcagcagaaggatgccagaaacgttcagctgcactcttaccascadmctggcattccttacaatccacagggacaagggattgttgaacgcactcatggcacattaaaagtcaatttacaaaaaataaaaaagggggagtcatatcccctgacgccccataattatctgtctcattctctctttattcaaaattttttgaccttggatgcccatggtaagagtgctgcagagcgcttttggcatccttccactgccactcaggctttggtcaaatggaaagacccacttacgggctcttggcaaggcccagatccagtcctcatatggggccgagggcatgtttgtgtttttccacaggatgcagaaggccctcggtggctgccagaacgattggtgcgacatgtggaccctctacctgctgatgacattgatgaccctaagcaatgcagaagaagaccagacgtattgggcctatgtacctga

SEQ ID NO: 51 - protein, 165 aa, synthetic construct
PKPEGQQKDARNVQLHSYXXXGIPYNPQGQGIVERTHGTLKVNLQKIKKGESYPLTPHNYLSHSLFIQNFLTLDAHGKSAAERFWHPSTATQALVKWKDPLTGSWQGPDPVLIWGRGHVCVFPQDAEGPRWLPERLVRHVDPLPADDIDDPKQCRRRPDVLGLCT

SEQ ID NO: 52 - DNA, 438 bp, synthetic construct
  source 1..438: mol_type = other DNA
tggacacatgaaacaacatttggaaagttttgtaaatcaggcactccctgcagtcaggtgactgatttacaagacgggactagagactggtctaagaaatctgttaatatatctgcttgtgttccttccccatatacacttttgattscadmttggtcaaatggaaagacccacttacaggctcttggcaaggcccagatccagtcctcatatggggccgagggcatgtttgtgtttttccacaggatgcagaaggccctcggtggttgccagaacgattggtgcgacatgtggaccctctacctgctgatgacattgatgaccctcagcaatacagaagaagaccagacgtattgggcctatgtacctgatccaccaatcctccaccctgttgtatgggaaggtcctgagattccagtscadmaaataaaactataa

SEQ ID NO: 53 - protein, 145 aa, synthetic construct
WTHETTFGKFCKSGTPCSQVTDLQDGTRDWSKKSVNISACVPSPYTLLIXXWSNGKTHLQALGKAQIQSSYGAEGMFVFFHRMQKALGGCQNDWCDMWTLYLLMTLMTLSNTEEDQTYWAYVPDPPILHPVVWEGPEIPVXXIKL

SEQ ID NO: 54 - DNA, 324 bp, synthetic construct
  source 1..324: mol_type = other DNA
cgccccataattacttgtctttttattcaaaattttttgactttggatgcctatgttaagagtgcagctgaacgtttctggcatccttctgccgaccctgaggctttggtcagaaagaaggatccacttactggatcatggcaaggcccagacccagtcctcatatggggccgagggcatgtttgtgtttttccacaggatgcagatagtcctcggtggctgccagaacgattggtgcgacatgtggaccctctacctgctgatgacattgatgaccctcagcaatgcagaagaagaccagacgtattgggcctacgtacctga

SEQ ID NO: 55 - protein, 107 aa, synthetic construct
RPIITCLFIQNFLTLDAYVKSAAERFWHPSADPEALVRKKDPLTGSWQGPDPVLIWGRGHVCVFPQDADSPRWLPERLVRHVDPLPADDIDDPQQCRRRPDVLGLRT

SEQ ID NO: 56 - DNA, 255 bp, synthetic construct
  source 1..255: mol_type = other DNA
accactgttgttaaaactgttaatatatctgcttgtgttccttccccttatatacttttgattaaaaatattaatgtacacscadmagaacaggtctggggtattttccccaggggtcatagatttacctgtactccaccaaaaaactacaaaggcaataatttggaaaacagatacacctgtgtggatagatcagtggccccttacacaggaaaagatatcggccgcccaggcgcttgtacaggagcagcttga

SEQ ID NO: 57 - protein, 84 aa, synthetic construct
TTVVKTVNISACVPSPYILLIKNINVHXXEQVWGIFPRGHRFTCTPPKNYKGNNLENRYTCVDRSVAPYTGKDIGRPGACTGAA

SEQ ID NO: 58 - DNA, 432 bp, synthetic construct
  source 1..432: mol_type = other DNA
ccttggatgcccatggtaagagtgctgcagagcgcttttggcatccttctactgccactcaggctttggtcaaatggaaagacccacttacaggctcttggcaaggcccagatccagtcctcatatggggccgagggcatgtttgtgtttttccacaggatgcagagggccctcggtggctgccaagacgattggtgcgacatgtggaccctctacctgctgatgacattgatgaccctcagcaatgcagaagaagaccagacgtattgggcctatgttcctgatccaccaatcctccaccctgctgtatgggaaggtcctgagattccagactatgtcaatgacactcacgccctaggattgccttctgatggacacataaaacaacatttggaaagttttgtaaatcaggcactccctgcagtcaggtga

SEQ ID NO: 59 - protein, 143 aa, synthetic construct
PWMPMVRVLQSAFGILLLPLRLWSNGKTHLQALGKAQIQSSYGAEGMFVFFHRMQRALGGCQDDWCDMWTLYLLMTLMTLSNAEEDQTYWAYVPDPPILHPAVWEGPEIPDYVNDTHALGLPSDGHIKQHLESFVNQALPAVR

SEQ ID NO: 60 - DNA, 285 bp, synthetic construct
  source 1..285: mol_type = other DNA
ctttggatgcccatggtaaaagtgcagctgcacgttttttggcatccttcaactagccctcaggccttggtcaaatggaaggacccacttacgggtgtctggcaaggcccagatccagtcctcatatggggccgagggcatgtttgtgtttttccacaggatgcagatagtcctcggtggctgctagaacgattggtgcgacatgtggaccctctacctgctgatgacattgatgaccctcagcaatgcagaagaagaccagacgtattgggcctacgtacctga

SEQ ID NO: 61 - protein, 94 aa, synthetic construct
LWMPMVKVQLHVFWHPSTSPQALVKWKDPLTGVWQGPDPVLIWGRGHVCVFPQDADSPRWLLERLVRHVDPLPADDIDDPQQCRRRPDVLGLRT

SEQ ID NO: 62 - DNA, 792 bp, synthetic construct
  source 1..792: mol_type = other DNA
tggacacatgaaacaacatttggaaagttttgtaaatcaggcactccctgcagtcaggtgactgacttacaagacgggactagagactggtctaagaaatctgttaatgtatctgcttgtgttccttccccttatacacttttgattgaaaatattaatgtacattttgtaggagttcagtttatggaagatgtgattcagagtataaaagttaaatcttatttagaatgtcattcagaatatcattggatacgtgttacttctaaaaggtataataatagtcaatatgattggaatcgggttcgtttacatcttcaaggaatttggcatgatgctaatgtgtctttagatascadmcgaggagtgcagatagagccggcggcggcggcgcagcgagcgagcagtgaccgcgctcctacccagttctgccccacggctcctacctgcttgcctccctcagcccctcgcccggctgtgactaaccgcgaccatgatgttctccagcttcaacgccgactacgacgcggcctcttcccgctgcagcagcgcctccccagctggggacagtctctcctactaccactcacccgccgactccttctccagcatgggctctcctgtcaatgcgcaggtaaggctggcttcaccgagcccagggctcggggtcactggggtggaggcatcgggcgggaagctcaggaagacgagtcgggtaccccttttggcggggagggagcagccctaactcgcgagtcccggacttgtggggcgctcacacacgcttgtcagtaa

SEQ ID NO: 63 - protein, 263 aa, synthetic construct
WTHETTFGKFCKSGTPCSQVTDLQDGTRDWSKKSVNVSACVPSPYTLLIENINVHFVGVQFMEDVIQSIKVKSYLECHSEYHWIRVTSKRYNNSQYDWNRVRLHLQGIWHDANVSLDXXRGVQIEPAAAAQRASSDRAPTQFCPTAPTCLPPSAPRPAVTNRDHDVLQLQRRLRRGLFPLQQRLPSWGQSLLLPLTRRLLLQHGLSCQCAGKAGFTEPRARGHWGGGIGREAQEDESGTPFGGEGAALTRESRTCGALTHACQ

SEQ ID NO: 64 - DNA, 327 bp, synthetic construct
  source 1..327: mol_type = other DNA
cagacctcctgccctggcggatgccatggattccagagccctagtctcccacccctcactgtcgcaggacagtctgggcatgtttgcacatgctcctgctgcacagggcactctctcgtaatgtatctcagagttcagtcccatagatggccttataacgtaagtactcttctaagcactgaaggacattatcatccactttggggtcaaacttgttggccaacaggtgagggttacgaagaatccagtgcaggtccccagccccataaatgcagataccccgctggtgggttccagagcaaggtccataaggtgcccccttactga

SEQ ID NO: 65 - protein, 108 aa, synthetic construct
QTSCPGGCHGFQSPSLPPLTVAGQSGHVCTCSCCTGHSLVMYLRVQSHRWPYNVSTLLSTEGHYHPLWGQTCWPTGEGYEESSAGPQPHKCRYPAGGFQSKVHKVPPY

SEQ ID NO: 66 - DNA, 420 bp, synthetic construct
  source 1..420: mol_type = other DNA
gagaaagtctcagagcgacaatggccagcaggaaatagcagcccagagcccacaggtagtgcttctggaagagtttcttcttccaccaaatcatcttcatggaatggaagatcggtagaatttgggcaccaggaagaagaaggatgggatccttscadmaccctggccgcgggggcggcgcgcaccgtccacgcgtccggggcccagcggggccgggcccggagtcggcatgaatcgctgctgggcgctcttcctgtctctctgctgctacctgcgtctggtcagcgccgaggtgagttgcgacagccgtggggctggttcgcttcattcattgcccccacccccatccctgttgccccctcccctccctgcagtgaactttggacccttgcagcccgtgggcctggcgcccggcgctag

SEQ ID NO: 67 - protein, 139 aa, synthetic construct
EKVSERQWPAGNSSPEPTGSASGRVSSSTKSSSWNGRSVEFGHQEEEGWDPXXTLAAGAARTVHASGAQRGRARSRHESLLGALPVSLLLPASGQRRGELRQPWGWFASFIAPTPIPVAPSPPCSELWTLAARGPGARR

SEQ ID NO: 68 - DNA, 309 bp, synthetic construct
  source 1..309: mol_type = other DNA
tcttatcccccacctcctcagaaaccccagaataagcccctaactggcctaagggagagggggtggggtggtgccgagggtgcagaaggcggcgcgtccttccaagcccacttcagttccagcttaggttctgtccgggaaccggcttgcacggaaggtgcgagctcgcgcactggtggcagccacgccaacctacggcaggggtttgcgtcccaccctggctcccgctccagctcttgcttgctcggccccagagcgtggtgcaggagcagcttgtgtcttgggcgcggcgggggtacagagagatag

SEQ ID NO: 69 - protein, 102 aa, synthetic construct
SYPPPPQKPQNKPLTGLRERGWGGAEGAEGGASFQAHFSSSLGSVREPACTEGASSRTGGSHANLRQGFASHPGSRSSSCLLGPRAWCRSSLCLGRGGGTER

SEQ ID NO: 70 - DNA, 762 bp, synthetic construct
  source 1..762: mol_type = other DNA
ggtgaagaggctcaggggctgcgaggagcgctacgcgcctggtcccgtcccgcctcagctcggcggccgccgggagcccgcaccgagccggctcctgggagggccggcccctctcgggcctccaacgaggagcaggaaggaggcggcggcggcggcgaaggggttaaggtgaagggcttcgaggccgcggccgggccttgggccgcagccagcgcaggttgttttgaccacggaggagccgtctccgtctccttttgttctcggggctcctcgagggccgccggccgtccgccctggggccccgcccttccgcggccgtcccccgtggcccgcacccgggagggaggacgcggggatcagcctggctgcctgcagtcccctcccgacgccccctcctctcctcctgctgatgccccccgggccgcggccagctgttggggcggggggcgccggccggccccagctgccgcctcgccgcggggcctgggggctgggccctgtgccagggcgtcctgggaacggcggcgccccagccgctgctctccgcagcccaccccgcccggccccccgactcgctcactcaccccacgcatgcacactcttggccggaggcgatgctgcgctccggcgggcgggcgcgcagggcgacgggcacgcactggcgcggccgggtcgcgcgcccgccgccacgcccgtgcacatgcgggacacacgcgcgcgcactacacacacacacgcatggtccccgcacacacggcttga

SEQ ID NO: 71 - protein, 253 aa, synthetic construct
GEEAQGLRGALRAWSRPASARRPPGARTEPAPGRAGPSRASNEEQEGGGGGGEGVKVKGFEAAAGPWAAASAGCFDHGGAVSVSFCSRGSSRAAGRPPWGPALPRPSPVARTREGGRGDQPGCLQSPPDAPSSPPADAPRAAASCWGGGRRPAPAAASPRGLGAGPCARASWERRRPSRCSPQPTPPGPPTRSLTPRMHTLGRRRCCAPAGGRAGRRARTGAAGSRARRHARAHAGHTRAHYTHTRMVPAHTA

SEQ ID NO: 72 - DNA, 318 bp, synthetic construct
  source 1..318: mol_type = other DNA
gcttccgagggggctcccaccccccactgttctgtgctctttgctgatcccagccagcacgctgcagagaggctgggtgacagctggataaggctttcccgcctgcccttaccattcccagcttcatccagcacctcctcctcctttcccacaactccctgggtgtgtgtttggggggtgagcctatggcacagaaactggtgcctgtctcctcactttaatcacagcatccttggacacatggctctcaggaacccacagttgtgtggtgctttgcagtttacgaagcactttcctgctaagccttactctgagtaa

SEQ ID NO: 73 - protein, 105 aa, synthetic construct
ASEGAPTPHCSVLFADPSQHAAERLGDSWIRLSRLPLPFPASSSTSSSFPTTPWVCVWGVSLWHRNWCLSPHFNHSILGHMALRNPQLCGALQFTKHFPAKPYSE

SEQ ID NO: 74 - DNA, 243 bp, synthetic construct
  source 1..243: mol_type = other DNA
tcctcagcccagggtagcctagaatggccacactgctcttcaccaggcatcctcattcgagcccccccggccccccatcttgagagacaagcatatctttcttttccatgtcttgggctgccaatattggacaggacagaggggaagaaacagaaggaaaatcagatcgcaaggcttctgtgtatcttgagcaggcctgggcctcagttgccgccgcgtgagaatatgagaaggttggattag

SEQ ID NO: 75 - protein, 80 aa, synthetic construct
SSAQGSLEWPHCSSPGILIRAPPAPHLERQAYLSFPCLGLPILDRTEGKKQKENQIARLLCILSRPGPQLPPRENMRRLD

SEQ ID NO: 76 - DNA, 420 bp, synthetic construct
  source 1..420: mol_type = other DNA
gcaatccaggtttccttggcagctgaagctctacagtttctctgcctctccactgttgacatttggggccagacagttcttgattgtgggggaggctgtcctgtgcatagtaggatgtttagcagcaaccctggcctctacctactagacaccagtagcaggcctccagttgtgataaccaaaagtgcctccagacctggccagtgtcccctgggggtcactcactccctgctctatgacctccactgggtgaagagtggacctgaactgaaaacagtccatgaaagagggaggggcccgctctgctccttaccagtcgtgttgacctttagccatttacttaatttttctaagcctcagcttcctcatttggaagacagggatacaaacagtgacagcctcttgattgtatttgattga

SEQ ID NO: 77 - protein, 139 aa, synthetic construct
AIQVSLAAEALQFLCLSTVDIWGQTVLDCGGGCPVHSRMFSSNPGLYLLDTSSRPPVVITKSASRPGQCPLGVTHSLLYDLHWVKSGPELKTVHERGRGPLCSLPVVLTFSHLLNFSKPQLPHLEDRDTNSDSLLIVFD

SEQ ID NO: 78 - DNA, 243 bp, synthetic construct
  source 1..243: mol_type = other DNA
gggaatcatgcaggagagaaccccagggagaaggggagagtccttcatgcattttaccagtgtttagtgagcacctactctgtgctttcccccagtctctgtcctgggctcttccccgtgcaggctgggagggtggggttctgggtttgtttccataagacatcatcgtctcttttttattataggccgggtccagggtgtccactgggcccagctgggatctgcctactctgccatggctag

SEQ ID NO: 79 - protein, 80 aa, synthetic construct
GNHAGENPREKGRVLHAFYQCLVSTYSVLSPSLCPGLFPVQAGRVGFWVCFHKTSSSLFYYRPGPGCPLGPAGICLLCHG

SEQ ID NO: 80 - DNA, 279 bp, synthetic construct
  source 1..279: mol_type = other DNA
cagctgcagccagctctccagtgggcaaggaggtcttggcatgagtgttacgtgccatttggtactgggtcttcagtccgctctcctaagaggttaattgattcattatgccacaaacagcctgggagacctggctgggcacccccacttcggcttcctctgctgctgcctctcctgccaaccccagacagaattagaattaaaatcaaatcaaatggctacaaccccctcagttcacaggtgatagccaggacccgagaggggcagcaaccaacctga

SEQ ID NO: 81 - protein, 92 aa, synthetic construct
QLQPALQWARRSWHECYVPFGTGSSVRSPKRLIDSLCHKQPGRPGWAPPLRLPLLLPLLPTPDRIRIKIKSNGYNPLSSQVIARTREGQQPT

SEQ ID NO: 82 - DNA, 279 bp, synthetic construct
  source 1..279: mol_type = other DNA
agggtggggctgtgggaggggaggcaggcagggagaaggtgcccagggcatctgcaccctgagtatccaggtgtggactcagccagggagggtggtgctggaggagccacctccctgtctctctggccaaaggcccgctctacaaggtctcccggggacacctggccgggaccagtgggcagccctgcccgtgcccaagagggcactcagagaatgggcacgtgcttggtggcacacacgtggcagggctggcgggctgtgtcgggaatgtatttataa

SEQ ID NO: 83 - protein, 92 aa, synthetic construct
RVGLWEGRQAGRRCPGHLHPEYPGVDSAREGGAGGATSLSLWPKARSTRSPGDTWPGPVGSPARAQEGTQRMGTCLVAHTWQGWRAVSGMYL

SEQ ID NO: 84 - DNA, 243 bp, synthetic construct
  source 1..243: mol_type = other DNA
acgctgtcttcagagcaaattccattctattctaacctctggcctgttccctggagccctggtcagcacccccctgcacccccagctccccttccctctggggttttgtctctttgtcactttgtaatccttgcccagactgctatctacgggggacagcatttcctgcctttgtttcctctcccagttgggcccctggctccctctcaaaagcattccccgggccctttcaaacccgcctag

SEQ ID NO: 85 - protein, 80 aa, synthetic construct
TLSSEQIPFYSNLWPVPWSPGQHPPAPPAPLPSGVLSLCHFVILAQTAIYGGQHFLPLFPLPVGPLAPSQKHSPGPFKPA

SEQ ID NO: 86 - DNA, 474 bp, synthetic construct
  source 1..474: mol_type = other DNA
cttttctgctgtttctttccaaggtccttcgcccccaccctcatattgcccctccacaccccgggtgggggtcgggtcggagaagacgaggttttcaatagcaggcctgtttcgaggcaaccatgtggctattttttcctaatcaacttaacctttccacaaagcacatcttttccccatctcctcccaaccagggacattccagaaatggcagagagaaaggaatggagccagagggacagacagacacactgttcgtgggacaataggctagacggaagtgcatcagttttaggaaagtctgctctaaacagggccccttgggagcccacagggacgagcaatagttttgtcatgggcagtggcagtgggatggggagacagtgtgaccctgagatgctgtgtggagggggacagagcttgtccccgacacccttcagtgtatttgctggctttcagccatcagagagctag

SEQ ID NO: 87 - protein, 157 aa, synthetic construct
LFCCFFPRSFAPTLILPLHTPGGGRVGEDEVFNSRPVSRQPCGYFFLINLTFPQSTSFPHLLPTRDIPEMAERKEWSQRDRQTHCSWDNRLDGSASVLGKSALNRAPWEPTGTSNSFVMGSGSGMGRQCDPEMLCGGGQSLSPTPFSVFAGFQPSES

SEQ ID NO: 88 - DNA, 237 bp, synthetic construct
  source 1..237: mol_type = other DNA
aagagtctgcccaccattcaacgtcaagctcaaagttcccctgtccagccctcactttccgcagccggcttccggctgcctctacccagagggatgtctccaaggagtgctgatggtgctgagatgagggcctccaggctagagaagggagctgtagttgtgaccttaggaataaatgtacagcttagggcaggcatggggcaaaaggtcagagggagagagacagaaacacaatga

SEQ ID NO: 89 - protein, 78 aa, synthetic construct
KSLPTIQRQAQSSPVQPSLSAAGFRLPLPRGMSPRSADGAEMRASRLEKGAVVVTLGINVQLRAGMGQKVRGRETETQ

SEQ ID NO: 90 - DNA, 312 bp, synthetic construct
  source 1..312: mol_type = other DNA
gggctcccctatcccagcagttccagctccctacctctctctgcctttagtccccaccccaccccaccccacccctctccttcccaccctctctcccgcccaactgaaccattgtcaggggctccacaggggctgtgtccagggcatgctggtcccccctggggactatgggaatttctccattcagcacttcctatgggaacgctgggtggaggggcactggaaagtggcctcagagctctgggtccttgccctgccctggaggccgaggagggttcgcttacagtagcaaaagggaacggttatttttaa

SEQ ID NO: 91 - protein, 103 aa, synthetic construct
GLPYPSSSSSLPLSAFSPHPTPPHPSPSHPLSRPTEPLSGAPQGLCPGHAGPPWGLWEFLHSALPMGTLGGGALESGLRALGPCPALEAEEGSLTVAKGNGYF

SEQ ID NO: 92 - DNA, 315 bp, synthetic construct
  source 1..315: mol_type = other DNA
ggggcaagaggcaatccttccgtctgtcccagagcccccactggagtccccagcccgtggtatgaccagccagcacttgtcacagtgcttctgactgtgccttctcttgcagatgaagacggggctgagttggacctgaatttgactcagtcccattctggaggcaagctggagagcttatcccgagggagaaggagcctaggtaagaatgagggtgcaaacgggggcccctcaaaggtgggggccagggaagaagaactgagcacacagcctgccggaggctgtgagggtgggccctgtttgtcccacacttag

SEQ ID NO: 93 - protein, 104 aa, synthetic construct
GARGNPSVCPRAPTGVPSPWYDQPALVTVLLTVPSLADEDGAELDLNLTQSHSGGKLESLSRGRRSLGKNEGANGGPSKVGAREEELSTQPAGGCEGGPCLSHT

SEQ ID NO: 94 - DNA, 231 bp, synthetic construct
  source 1..231: mol_type = other DNA
gggctgtggcctgggacagcaggcatggagcaagcctgggacctgcctcctgctgtactgcagaaaccaaaaggagaatgtagatcagggaaggcaagtgcccactccacgcccctcttcctctgtgcccacctgcagtccccaaaacactgtagacagtggctggggggcctccaggtaagagtcagtggcctgagttccactctttgctctgtgaatttgggcatctaa

SEQ ID NO: 95 - protein, 76 aa, synthetic construct
GLWPGTAGMEQAWDLPPAVLQKPKGECRSGKASAHSTPLFLCAHLQSPKHCRQWLGGLQVRVSGLSSTLCSVNLGI

SEQ ID NO: 96 - DNA, 435 bp, synthetic construct
  source 1..435: mol_type = other DNA
cagccagtacttaaaacctcccctgacgtggaaggaagagggaatcggccaaccgttttggaggtgctccacttcctgccggctaggggccctgagcagccctccaccccactcctgacggagttccctctcccttcagattcccagccggtcgccgagccagccatgatcgcggagtgtaaaacgcgcactgaggtgtttgagatctcccggcgcctggttgaccgcaccaacgccaacttcctggtgtggccgccctgcgtggaggtgcagcgctgctccggctgctgcaacaatcgcaacgtgcagtgccgccccacccaggtgcagctgcgacatgtccaggtgtgcaggccccacgtcccctcctgggctggcccagctgagagcaggggctgcccctctggggctggcactcacggaccaggctcttga

SEQ ID NO: 97 - protein, 144 aa, synthetic construct
QPVLKTSPDVEGRGNRPTVLEVLHFLPARGPEQPSTPLLTEFPLPSDSQPVAEPAMIAECKTRTEVFEISRRLVDRTNANFLVWPPCVEVQRCSGCCNNRNVQCRPTQVQLRHVQVCRPHVPSWAGPAESRGCPSGAGTHGPGS

SEQ ID NO: 98 - DNA, 273 bp, synthetic construct
  source 1..273: mol_type = other DNA
atgcgtcaaaaggcattcctggcagggtgtgggctcagtccagagaaggcgctctcaggaagctctccggacaggtgtgcggaggctgcccaagaatcctctatggcctcccaagccactgtgacaaaaagtcacaggcagacctccagacaggctgggtatgggacattaagtaaaaggcattgcctcattctttacagggataaaatcccaaaatgtctcttgaagagacatgtctacaaacatattggaccctcaggatgttctgggtag

SEQ ID NO: 99 - protein, 90 aa, synthetic construct
MRQKAFLAGCGLSPEKALSGSSPDRCAEAAQESSMASQATVTKSHRQTSRQAGYGTLSKRHCLILYRDKIPKCLLKRHVYKHIGPSGCSG

SEQ ID NO: 100 - DNA, 555 bp, synthetic construct
  source 1..555: mol_type = other DNA
agcctcccctcacttccttccagacaaccatctcccgcctctgccacagcccctgaccttggctggcgctccaggaatgaggacaccacaggctccacgctccacccggaaatgcctttctccctctctgagagcaccgaggggggctgtggccaagctggaggccaggtcgggagggcttgttttgatggaaaagctacaagaagggcagagggcaaggtcctgctattgttttggccgcagtgtctgcactgctgctcttcaggctttcgaggaaagattccccacagaggacgctggggtgggaagagaaggcaggcagctacctcagcccctgcccaagtggtcttacagaggcacttgggtggttctgtcctccaggtgaggaagatcgagattgtacggaagaagccaagctttaagaaggccacagtgaccctggaggaccacctggcgtgcaagtgtgagacggtagtggctgcacgacctgtgacccgaagcccagggagttcccaggagcagcgaggtaaccttcagtccagggttggtctctga

SEQ ID NO: 101 - protein, 184 aa, synthetic construct
SLPSLPSRQPSPASATAPDLGWRSRNEDTTGSTLHPEMPFSLSESTEGGCGQAGGQVGRACFDGKATRRAEGKVLLLFWPQCLHCCSSGFRGKIPHRGRWGGKRRQAATSAPAQVVLQRHLGGSVLQVRKIEIVRKKPSFKKATVTLEDHLACKCETVVAARPVTRSPGSSQEQRGNLQSRVGL

SEQ ID NO: 102 - DNA, 279 bp, synthetic construct
  source 1..279: mol_type = other DNA
tattgttcccctcgtccgtctgtctcgatgcctgattcggacggccaatggtgcttccccgcccctccacgcgtccgtccacccctctgccagtgggtctcccctcagtggcscadmctcaggggctgcgaggagcgctacgcgcctggtcccgtcccgcctcagctcggcggccgccgggagcccgcaccgagccggctcctgggagggccggcccctctcgggcctccaacgaggagcaggaaggaggcggcggcggcggcgaaggggttaaggtga

SEQ ID NO: 103 - protein, 92 aa, synthetic construct
YCSPRPSVSMPDSDGQWCFPAPPRVRPPLCQWVSPQWXXLRGCEERYAPGPVPPQLGGRREPAPSRLLGGPAPLGPPTRSRKEAAAAAKGLR

SEQ ID NO: 104 - DNA, 627 bp, synthetic construct
  source 1..627: mol_type = other DNA
agggcttcgaggccgcggccgggccttgggccgcagccagcgcaggttgttttgaccacggaggagccgtctccgtctccttttgttctcggggctcctcgagggccgccggccgtccgccctggggccccgcccttccgcggccgtcccccgtggcccgcacccgggagggaggacgcggggatcagcctggctgcctgcagtcccctcccgacgccccctcctctcctcctgctgatgccccccgggccgcggccagctgttggggcggggggcgccggccggccccagctgccgcctcgccgcggggcctgggggctgggccctgtgccagggcgtcctgggaacggcggcgccccagccgctgctctccgcagcccaccccgcccggccccccgactcgctcactcaccccacgcatgcacactcttggccggaggcgatgctgcgctccggcgggcgggcgcgcagggcgacgggcacgcactggcgcggccgggtcgcgcgcccgccgccacgcccgtgcacatgcgggacacacgcgcgcgcactacacacacacacgcatggtccccgcacacacggcttgagcacacgtgcgcgcacacccacgcacgcacagcctag

SEQ ID NO: 105 - protein, 208 aa, synthetic construct
RASRPRPGLGPQPAQVVLTTEEPSPSPFVLGAPRGPPAVRPGAPPFRGRPPWPAPGREDAGISLAACSPLPTPPPLLLLMPPGPRPAVGAGGAGRPQLPPRRGAWGLGPVPGRPGNGGAPAAALRSPPRPAPRLAHSPHACTLLAGGDAALRRAGAQGDGHALARPGRAPAATPVHMRDTRARTTHTHAWSPHTRLEHTCAHTHARTA

SEQ ID NO: 106 - DNA, 228 bp, synthetic construct
  source 1..228: mol_type = other DNA
ctctgtttccttcttgggtgttctgagggaggggaaacaggaaccctccctcgggtccctctccacagcacccatgggtgtgtttttttttttttttggtcaggtcagttccacaccctttgcgcattacccttctatgattgctttctttcagccactcccatgtggctgaaaatagtgtcgatgtgcttggggggtactgttcagagcatttctcccttcaagtaa

SEQ ID NO: 107 - protein, 75 aa, synthetic construct
LCFLLGCSEGGETGTLPRVPLHSTHGCVFFFFWSGQFHTLCALPFYDCFLSATPMWLKIVSMCLGGTVQSISPFK

SEQ ID NO: 108 - DNA, 279 bp, synthetic construct
  source 1..279: mol_type = other DNA
gcctatggcacagaaactggtgcctgtctcctcactttaatcacagcatccttggacacatggctctcaggaacccacagttgtgtggtgctttgcagtttacgaagcactttcctgctaagccttactctgagtaagcaagcctcaggcagctcttggggaagagacctaaagggaaaacctatcgacatggggaccagtccaggaaggtggacttcaggagatcttactggcagaggtggccttggggctggccacgtctcaggcctgtgtggctga

SEQ ID NO: 109 - protein, 92 aa, synthetic construct
AYGTETGACLLTLITASLDTWLSGTHSCVVLCSLRSTFLLSLTLSKQASGSSWGRDLKGKPIDMGTSPGRWTSGDLTGRGGLGAGHVSGLCG

SEQ ID NO: 110 - DNA, 429 bp, synthetic construct
  source 1..429: mol_type = other DNA
attaggcctgagactctcaggcaggctggcgcttggaggtatggtcggcttctgcctctgccaaccatagacaacgcccctgggtgctggggccaagagcgacgtcctctctcagctgaacggcgcactggggagtgtgtatctgtgtgcagagtgtgtgtctgtgtgcgctggggcccaggtggagggtggggtccaagcccctttgatctgccagcatggttgggagcaggtaattcacctggcctcacgcttcctaccttctgcagctggtgttgggggtggggtggggtggggaagagactgtttgccttggctcccaaggctggctgtgccccagctgccttctcgccacgccctcaccctgctaggaaccccaggcctgagatctgggacagtttcctcatagtaccaagcctcctttcctag

SEQ ID NO: 111 - protein, 142 aa, synthetic construct
IRPETLRQAGAWRYGRLLPLPTIDNAPGCWGQERRPLSAERRTGECVSVCRVCVCVRWGPGGGWGPSPFDLPAWLGAGNSPGLTLPTFCSWCWGWGGVGKRLFALAPKAGCAPAAFSPRPHPARNPRPEIWDSFLIVPSLLS

SEQ ID NO: 112 - DNA, 258 bp, synthetic construct
  source 1..258: mol_type = other DNA
gagttagttgtggtattatcaaacccagggcctcttagtgagttctgggcacccagtggtcaaattgctagaagcatgtgcaggaatgacctctctgctaagaataaagtggactctataggaaacaatttgcatgtgtggggggtggtatgggagactatcccaggtggtcctcctggtggaggaggtgagggaatcatgcaggagagaaccccagggagaaggggagagtccttcatgcattttaccagtgtttag

SEQ ID NO: 113 - protein, 85 aa, synthetic construct
ELVVVLSNPGPLSEFWAPSGQIARSMCRNDLSAKNKVDSIGNNLHVWGVVWETIPGGPPGGGGEGIMQERTPGRRGESFMHFTSV

SEQ ID NO: 114 - DNA, 270 bp, synthetic construct
  source 1..270: mol_type = other DNA
gcacctactctgtgctttcccccagtctctgtcctgggctcttccccgtgcaggctgggagggtggggttctgggtttgtttccataagacatcatcgtctcttttttattataggccgggtccagggtgtccactgggcccagctgggatctgcctactctgccatggctagcagctgcagccagctctccagtgggcaaggaggtcttggcatgagtgttacgtgccatttggtactgggtcttcagtccgctctcctaagaggttaa

SEQ ID NO: 115 - protein, 89 aa, synthetic construct
APTLCFPPVSVLGSSPCRLGGWGSGFVSIRHHRLFFIIGRVQGVHWAQLGSAYSAMASSCSQLSSGQGGLGMSVTCHLVLGLQSALLRG

SEQ ID NO: 116 - DNA, 390 bp, synthetic construct
  source 1..390: mol_type = other DNA
ggcactgagttgttagacccaaggttaaacagtggtaagtcaagtcagctgacaccctcccagggctcctcccacgagaccatgccgtcctgtgtgtttgtgcacacacgtgtgtgtttgtgcacacacgtgtgtgtttgcctgggagtgagtgcggaggtacagcagcatcttatgcattttctttgccctgaggcgctgcgtgtcagctttgtgtatctcagattctcatctgccctcacttctttctctagacctctggcttcagccccttgggtctccctggacagggggggatgtggctgcgtccttcctatcgggctgctctcatgtcattgtgggtcctgtggtttccctggaggaagcccagctccgagtggggcctgttaa

SEQ ID NO: 117 - protein, 129 aa, synthetic construct
GTELLDPRLNSGKSSQLTPSQGSSHETMPSCVFVHTRVCLCTHVCVCLGVSAEVQQHLMHFLCPEALRVSFVYLRFSSALTSFSRPLASAPWVSLDRGGCGCVLPIGLLSCHCGSCGFPGGSPAPSGAC

SEQ ID NO: 118 - DNA, 345 bp, synthetic construct
  source 1..345: mol_type = other DNA
cccggctgtccacctgtccatgtccaagaggccccgtgggaactttctgtaggggatagtgtctgttggggcgaagagggctgtggctggaaagtccttactcccagcgtgtttgcctggcagggggaccccattcctgaggaactctatgagatgctgagtgaccactcgatccgctccttcgatgacctccagcgcctgctgcacggagactccgtaggtaaattgaatcctcgcccagggctctggccctccactgagtcctcgcgtgccagggggtggggagtgggtgccgggcaagggccatcctctcttttgtgccatccagagacctgtggcagctga

SEQ ID NO: 119 - protein, 114 aa, synthetic construct
PGCPPVHVQEAPWELSVGDSVCWGEEGCGWKVLTPSVFAWQGDPIPEELYEMLSDHSIRSFDDLQRLLHGDSVGKLNPRPGLWPSTESSRARGWGVGAGQGPSSLLCHPETCGS

SEQ ID NO: 120 - DNA, 519 bp, synthetic construct
  source 1..519: mol_type = other DNA
gggaggacttggccacacctgtctggggcagggctgagtaggcggacgggctggtacctagggtgtgaggtgtggcaggagaagcatccacatgtggctctggcttggggtagagggtggggctgtgggaggggaggcaggcagggagaaggtgcccagggcatctgcaccctgagtatccaggtgtggactcagccagggagggtggtgctggaggagccacctccctgtctctctggccaaaggcccgctctacaaggtctcccggggacacctggccgggaccagtgggcagccctgcccgtgcccaagagggcactcagagaatgggcacgtgcttggtggcacacacgtggcagggctggcgggctgtgtcgggaatgtatttataaacgctgtcttcagagcaaattccattctattctaacctctggcctgttccctggagccctggtcagcacccccctgcacccccagctccccttccctctggggttttgtctctttgtcactttgtaa

SEQ ID NO: 121 - protein, 172 aa, synthetic construct
GRTWPHLSGAGLSRRTGWYLGCEVWQEKHPHVALAWGRGWGCGRGGRQGEGAQGICTLSIQVWTQPGRVVLEEPPPCLSGQRPALQGLPGTPGRDQWAALPVPKRALREWARAWWHTRGRAGGLCRECIYKRCLQSKFHSILTSGLFPGALVSTPLHPQLPFPLGFCLFVTL

SEQ ID NO: 122 - DNA, 405 bp, synthetic construct
  source 1..405: mol_type = other DNA
tccttgcccagactgctatctacgggggacagcatttcctgcctttgtttcctctcccagttgggcccctggctccctctcaaaagcattccccgggccctttcaaacccgcctaggccgggggctgatgatgcaggcaggagggggccccagctgggcccacctattgttcaccaggccccccacccgatgtctcccacacccccaccccatgcccgactggccagccctggccaacacaatggggcaacttccaaatttagcttttctgctgtttctttccaaggtccttcgcccccaccctcatattgcccctccacaccccgggtgggggtcgggtcggagaagacgaggttttcaatagcaggcctgtttcgaggcaaccatgtggctattttttcctaa

SEQ ID NO: 123 - protein, 134 aa, synthetic construct
SLPRLLSTGDSISCLCFLSQLGPWLPLKSIPRALSNPPRPGADDAGRRGPQLGPPIVHQAPHPMSPTPPPHARLASPGQHNGATSKFSFSAVSFQGPSPPPSYCPSTPRVGVGSEKTRFSIAGLFRGNHVAIFS

SEQ ID NO: 124 - DNA, 291 bp, synthetic construct
  source 1..291: mol_type = other DNA
acagggccccttgggagcccacagggacgagcaatagttttgtcatgggcagtggcagtgggatggggagacagtgtgaccctgagatgctgtgtggagggggacagagcttgtccccgacacccttcagtgtatttgctggctttcagccatcagagagctagaagagtctgcccaccattcaacgtcaagctcaaagttcccctgtccagccctcactttccgcagccggcttccggctgcctctacccagagggatgtctccaaggagtgctgatggtgctgagatga

SEQ ID NO: 125 - protein, 96 aa, synthetic construct
TGPLGSPQGRAIVLSWAVAVGWGDSVTLRCCVEGDRACPRHPSVYLLAFSHQRARRVCPPFNVKLKVPLSSPHFPQPASGCLYPEGCLQGVLMVLR

SEQ ID NO: 126 - DNA, 399 bp, synthetic construct
  source 1..399: mol_type = other DNA
atgtacagcttagggcaggcatggggcaaaaggtcagagggagagagacagaaacacaatgagggactgggagatggagagagaccaagacctagaaggacgctgggtgagggctcccctatcccagcagttccagctccctacctctctctgcctttagtccccaccccaccccaccccacccctctccttcccaccctctctcccgcccaactgaaccattgtcaggggctccacaggggctgtgtccagggcatgctggtcccccctggggactatgggaatttctccattcagcacttcctatgggaacgctgggtggaggggcactggaaagtggcctcagagctctgggtccttgccctgccctggaggccgaggagggttcgcttacagtag

SEQ ID NO: 127 - protein, 132 aa, synthetic construct
MYSLGQAWGKRSEGERQKHNEGLGDGERPRPRRTLGEGSPIPAVPAPYLSLPLVPTPPHPTPLLPTLSPAQLNHCQGLHRGCVQGMLVPPGDYGNFSIQHFLWERWVEGHWKVASELWVLALPWRPRRVRLQ

SEQ ID NO: 128 - DNA, 249 bp, synthetic construct
  source 1..249: mol_type = other DNA
agagcccagagtggggctgaaggccctccgagggtacagtctgggccccatcacctcctgaaccccatggccaccctggggtttgcctggagggcgcctcctcagaggcagggagccagaaggggagtatgttctctggagtggggtcccagtgaggggcaagaggcaatccttccgtctgtcccagagcccccactggagtccccagcccgtggtatgaccagccagcacttgtcacagtgcttctga

SEQ ID NO: 129 - protein, 82 aa, synthetic construct
RAQSGAEGPPRVQSGPHHLLNPMATLGFAWRAPPQRQGARRGVCSLEWGPSEGQEAILPSVPEPPLESPARGMTSQHLSQCF

SEQ ID NO: 130 - DNA, 318 bp, synthetic construct
  source 1..318: mol_type = other DNA
ccctattttataaaattggagactggagcccagagaagggaaagaagtggctgtggtgacacagctagcatgtggtacggctgggatcccaatagctcttctcagtgccgcctgctgtgtgtctctgctgtggctaagggctgtggcctgggacagcaggcatggagcaagcctgggacctgcctcctgctgtactgcagaaaccaaaaggagaatgtagatcagggaaggcaagtgcccactccacgcccctcttcctctgtgcccacctgcagtccccaaaacactgtagacagtggctggggggcctccaggtaa

SEQ ID NO: 131 - protein, 105 aa, synthetic construct
PYFIKLETGAQRRERSGCGDTASMWYGWDPNSSSQCRLLCVSAVAKGCGLGQQAWSKPGTCLLLYCRNQKENVDQGRQVPTPRPSSSVPTCSPQNTVDSGWGASR

SEQ ID NO: 132 - DNA, 291 bp, synthetic construct
  source 1..291: mol_type = other DNA
aatggatgggggctggcggaaggaaactggcatttacaacatgcagcagcctctgaattacctcacttgatcctgacagtggttcttgggtgtagacctcatcacccccacttgcacagggggaaacagattcagaacccatcagcgacctgcccaaataccatggctgataacagccagtacttaaaacctcccctgacgtggaaggaagagggaatcggccaaccgttttggaggtgctccacttcctgccggctaggggccctgagcagccctccaccccactcctga

SEQ ID NO: 133 - protein, 96 aa, synthetic construct
NGWGLAEGNWHLQHAAASELPHLILTVVLGCRPHHPHLHRGKQIQNPSATCPNTMADNSQYLKPPLTWKEEGIGQPFWRCSTSCRLGALSSPPPHS

SEQ ID NO: 134 - DNA, 390 bp, synthetic construct
  source 1..390: mol_type = other DNA
tcgcggagtgtaaaacgcgcactgaggtgtttgagatctcccggcgcctggttgaccgcaccaacgccaacttcctggtgtggccgccctgcgtggaggtgcagcgctgctccggctgctgcaacaatcgcaacgtgcagtgccgccccacccaggtgcagctgcgacatgtccaggtgtgcaggccccacgtcccctcctgggctggcccagctgagagcaggggctgcccctctggggctggcactcacggaccaggctcttgaatgcgtcaaaaggcattcctggcagggtgtgggctcagtccagagaaggcgctctcaggaagctctccggacaggtgtgcggaggctgcccaagaatcctctatggcctcccaagccactgtga

SEQ ID NO: 135 - protein, 129 aa, synthetic construct
SRSVKRALRCLRSPGAWLTAPTPTSWCGRPAWRCSAAPAAATIATCSAAPPRCSCDMSRCAGPTSPPGLAQLRAGAAPLGLALTDQALECVKRHSWQGVGSVQRRRSQEALRTGVRRLPKNPLWPPKPL

SEQ ID NO: 136 - DNA, 600 bp, synthetic construct
  source 1..600: mol_type = other DNA
tctggtgacttcaccacgccccctcccctgcggtcagctgtggcccttcctcttgcccaccttccatcccagggctgggccctgagcccgagattacgagtgtcactctccaccccacctcccactgccatggtatctcctgtccccaatgcttccagctctatggatggacacctgacagctgacctcccccttcccgcctccctcctggataaagcctcccctcacttccttccagacaaccatctcccgcctctgccacagcccctgaccttggctggcgctccaggaatgaggacaccacaggctccacgctccacccggaaatgcctttctccctctctgagagcaccgaggggggctgtggccaagctggaggccaggtcgggagggcttgttttgatggaaaagctacaagaagggcagagggcaaggtcctgctattgttttggccgcagtgtctgcactgctgctcttcaggctttcgaggaaagattccccacagaggacgctggggtgggaagagaaggcaggcagctacctcagcccctgcccaagtggtcttacagaggcacttgggtggttctgtcctccaggtga

SEQ ID NO: 137 - protein, 199 aa, synthetic construct
SGDFTTPPPLRSAVALPLAHLPSQGWALSPRLRVSLSTPPPTAMVSPVPNASSSMDGHLTADLPLPASLLDKASPHFLPDNHLPPLPQPLTLAGAPGMRTPQAPRSTRKCLSPSLRAPRGAVAKLEARSGGLVLMEKLQEGQRARSCYCFGRSVCTAALQAFEERFPTEDAGVGREGRQLPQPLPKWSYRGTWVVLSSR

SEQ ID NO: 138 - DNA, 231 bp, synthetic construct
  source 1..231: mol_type = other DNA
cccgaagcccagggagttcccaggagcagcgaggtaaccttcagtccagggttggtctctgattgctcagcctggccagccccttctcctgtggcagctgccgggtggggggaaattggatcaggcatgcgccccaccccccactcctggttaaattcatctgaagctttccatctcacagaacaatccagattcatccccccgactgcaaggccctatatgaggatgtag

SEQ ID NO: 139 - protein, 76 aa, synthetic construct
PEAQGVPRSSEVTFSPGLVSDCSAWPAPSPVAAAGWGEIGSGMRPTPHSWLNSSEAFHLTEQSRFIPPTARPYMRM

SEQ ID NO: 140 - DNA, 327 bp, synthetic construct
  source 1..327: mol_type = other DNA
tcagaaccctgggctaaaatttctgctctgtcacttgtgagttgtacgacaaccttgagctggctcgggctttgccagtccagtgtcctgctggtggcctggtctctggatcagaaactccaggccctcaatggttcttctgggtacaaaggtcccaagtccctgaattgcagagatagggtaactactttatgggagcttgtgtctgcaaggtgggaggtcaagtgtttaacccaaagagtggggtgggccttgagcttggcagagaaagctttcattttctacttgggggcccaggaggaagagagatgtaagcgcaaaccttga

SEQ ID NO: 141 - protein, 108 aa, synthetic construct
SEPWAKISALSLVSCTTTLSWLGLCQSSVLLVAWSLDQKLQALNGSSGYKGPKSLNCRDRVTTLWELVSARWEVKCLTQRVGWALSLAEKAFIFYLGAQEEERCKRKP

SEQ ID NO: 142 - DNA, 273 bp, synthetic construct
  source 1..273: mol_type = other DNA
gtctgtgggcggatggggctcagctgggtggttctactgctgtctctcatagtttcggtcagtcatctggaggccacactgggacagctgggcctctgtcattcagggcctctcttttccatatggtctccccagcagggtaaccagacttcttatgtggcggcacagggctccacaaagtgcaaaggtgggacctaccaggcctttttaggcttatgcctggacctggcacagcactgctctgcctccttttattgtttaacagatagatag

SEQ ID NO: 143 - protein, 90 aa, synthetic construct
VCGRMGLSWVVLLLSLIVSVSHLEATLGQLGLCHSGPLFSIWSPQQGNQTSYVAAQGSTKCKGGTYQAFLGLCLDLAQHCSASFYCLTDR

SEQ ID NO: 144 - DNA, 387 bp, synthetic construct
  source 1..387: mol_type = other DNA
gcgtggcatggagttcctaggctgcttctgaccccgtgttcctctgcttaccttacagggttatttaatatggtatttgctgtattgcccccatggggtccttggagtgataatattgttcccctcgtccgtctgtctcgatgcctgattcggacggccaatggtgcttccccgcccctccacgcgtccgtccacccctctgccagtgggtctcccctcagtggcscadmctcaggggctgcgaggagcgctacgcgcctggtcccgtcccgcctcagctcggcggccgccgggagcccgcaccgagccggctcctgggagggccggcccctctcgggcctccaacgaggagcaggaaggaggcggcggcggcggcgaaggggttaa

SEQ ID NO: 145 - protein, 128 aa, synthetic construct
AWHGVPRLLLTPCSSAYLTGLFNMVFAVLPPWGPWSDNIVPLVRLSRCLIRTANGASPPLHASVHPSASGSPLSGXXSGAARSATRLVPSRLSSAAAGSPHRAGSWEGRPLSGLQRGAGRRRRRRRRG

SEQ ID NO: 146 - DNA, 630 bp, synthetic construct
  source 1..630: mol_type = other DNA
tgccccccgggccgcggccagctgttggggcggggggcgccggccggccccagctgccgcctcgccgcggggcctgggggctgggccctgtgccagggcgtcctgggaacggcggcgccccagccgctgctctccgcagcccaccccgcccggccccccgactcgctcactcaccccacgcatgcacactcttggccggaggcgatgctgcgctccggcgggcgggcgcgcagggcgacgggcacgcactggcgcggccgggtcgcgcgcccgccgccacgcccgtgcacatgcgggacacacgcgcgcgcactacacacacacacgcatggtccccgcacacacggcttgagcacacgtgcgcgcacacccacgcacgcacagcctagcgccaggtgcccacccccgcgccacaggtgggcccacggtaggccctggaacctcgtcaactctagtgactctgtttccttcttgggtgttctgagggaggggaaacaggaaccctccctcgggtccctctccacagcacccatgggtgtgtttttttttttttttggtcaggtcagttccacaccctttgcgcattacccttctatgattgctttctttcagccactcccatgtggctga

SEQ ID NO: 147 - protein, 209 aa, synthetic construct
CPPGRGQLLGRGAPAGPSCRLAAGPGGWALCQGVLGTAAPQPLLSAAHPARPPDSLTHPTHAHSWPEAMLRSGGRARRATGTHWRGRVARPPPRPCTCGTHARALHTHTHGPRTHGLSTRARTPTHAQPSARCPPPRHRWAHGRPWNLVNSSDSVSFLGVLREGKQEPSLGSLSTAPMGVFFFFFGQVSSTPFAHYPSMIAFFQPLPCG

SEQ ID NO: 148 - DNA, 342 bp, synthetic construct
  source 1..342: mol_type = other DNA
agggaaaacctatcgacatggggaccagtccaggaaggtggacttcaggagatcttactggcagaggtggccttggggctggccacgtctcaggcctgtgtggctgagcctcaggtagagggtagaggcctcagcagctgggaaggagggttgggacggctgaggcagggcctggcagggggtcagctgaggcctgtgaggttccacctccatcagctgaactggcttcaggagagtgactcccactgtcacgtgaggcctcctgccttagcacccttctgctgggaaagagtgaaggggcactaccgcccttcaccacccagcttccttctggtttgctaa

SEQ ID NO: 149 - protein, 113 aa, synthetic construct
RENLSTWGPVQEGGLQEILLAEVALGLATSQACVAEPQVEGRGLSSWEGGLGRLRQGLAGGQLRPVRFHLHQLNWLQESDSHCHVRPPALAPFCWERVKGHYRPSPPSFLLVC

SEQ ID NO: 150 - DNA, 333 bp, synthetic construct
  source 1..333: mol_type = other DNA
tgccttaggtggtgggagaccaacttgctggaatctcccagccctagacgtgtctgcaaggttaagatcaaacagaatttggagctctggtgcaaagctaggaacagtgcgtgcatgcgcatgagagagagagagagagagagagagagagagagagagagagagagagccctcttcagcaggagtggtaaagaggtgtttaccatgggcctcataaatctctcaaagtcttcccccccaacccacccggttgaaatgccccttctagacagctattttcattttctggtttatttagttgtttattatctgttttttctcactggagtgtaa

SEQ ID NO: 151 - protein, 110 aa, synthetic construct
CLRWWETNLLESPSPRRVCKVKIKQNLELWCKARNSACMRMRERERERERERERERALFSRSGKEVFTMGLINLSKSSPPTHPVEMPLLDSYFHFLVYLVVYYLFFLTGV

SEQ ID NO: 152 - DNA, 264 bp, synthetic construct
  source 1..264: mol_type = other DNA
cagtttttctgctcaagggagaggtggggagcccagtgggaggctgggctcacattaaggaggggtgggggggggagggcctctggagcactaggaaagggaaatggtaggtgggaaaggctgggtctaaatggcttctgtggtctgcccagaggaggcgtcttcaaagggcttggctttggcgttgaatctaaattaggcctgagactctcaggcaggctggcgcttggaggtatggtcggcttctgcctctgccaaccatag

SEQ ID NO: 153 - protein, 87 aa, synthetic construct
QFFCSRERWGAQWEAGLTLRRGGGGRASGALGKGNGRWERLGLNGFCGLPRGGVFKGLGFGVESKLGLRLSGRLALGGMVGFCLCQP

SEQ ID NO: 154 - DNA, 339 bp, synthetic construct
  source 1..339: mol_type = other DNA
acaacgcccctgggtgctggggccaagagcgacgtcctctctcagctgaacggcgcactggggagtgtgtatctgtgtgcagagtgtgtgtctgtgtgcgctggggcccaggtggagggtggggtccaagcccctttgatctgccagcatggttgggagcaggtaattcacctggcctcacgcttcctaccttctgcagctggtgttgggggtggggtggggtggggaagagactgtttgccttggctcccaaggctggctgtgccccagctgccttctcgccacgccctcaccctgctaggaaccccaggcctgagatctgggacagtttcctcatag

SEQ ID NO: 155 - protein, 112 aa, synthetic construct
TTPLGAGAKSDVLSQLNGALGSVYLCAECVSVCAGAQVEGGVQAPLICQHGWEQVIHLASRFLPSAAGVGGGVGWGRDCLPWLPRLAVPQLPSRHALTLLGTPGLRSGTVSS

SEQ ID NO: 156 - DNA, 228 bp, synthetic construct
  source 1..228: mol_type = other DNA
gaaacaatttgcatgtgtggggggtggtatgggagactatcccaggtggtcctcctggtggaggaggtgagggaatcatgcaggagagaaccccagggagaaggggagagtccttcatgcattttaccagtgtttagtgagcacctactctgtgctttcccccagtctctgtcctgggctcttccccgtgcaggctgggagggtggggttctgggtttgtttccataa

SEQ ID NO: 157 - protein, 75 aa, synthetic construct
ETICMCGGWYGRLSQVVLLVEEVRESCRREPQGEGESPSCILPVFSEHLLCAFPQSLSWALPRAGWEGGVLGLFP

SEQ ID NO: 158 - DNA, 324 bp, synthetic construct
  source 1..324: mol_type = other DNA
gtgcggaggtacagcagcatcttatgcattttctttgccctgaggcgctgcgtgtcagctttgtgtatctcagattctcatctgccctcacttctttctctagacctctggcttcagccccttgggtctccctggacagggggggatgtggctgcgtccttcctatcgggctgctctcatgtcattgtgggtcctgtggtttccctggaggaagcccagctccgagtggggcctgttaaagtgcttattaagtttcaagtgtttttggtaacaggccagagaggctctaaaaatagggtttgcctgggcaccgggcatgggtaa

SEQ ID NO: 159 - protein, 107 aa, synthetic construct
VRRYSSILCIFFALRRCVSALCISDSHLPSLLSLDLWLQPLGSPWTGGDVAASFLSGCSHVIVGPVVSLEEAQLRVGPVKVLIKFQVFLVTGQRGSKNRVCLGTGHG

SEQ ID NO: 160 - DNA, 294 bp, synthetic construct
  source 1..294: mol_type = other DNA
cagacgctggctgtcatctgtcaggtgtggaggagaagcataaagattgtggggtttcccggaacctgtagtgtgatgagggagatggatgtatacaatcaatcagagcaaactgggggtcctctttggaggcgagggatacagcatcctctctgggtcttcaaggcttcggcagattctggcccttgggcctttgtgttcctggttctcaggcctggaatctacctcctgcccacccctagcccggctgtccacctgtccatgtccaagaggccccgtgggaactttctgtag

SEQ ID NO: 161 - protein, 97 aa, synthetic construct
QTLAVICQVWRRSIKIVGFPGTCSVMREMDVYNQSEQTGGPLWRRGIQHPLWVFKASADSGPWAFVFLVLRPGIYLLPTPSPAVHLSMSKRPRGNFL

SEQ ID NO: 162 - DNA, 252 bp, synthetic construct
  source 1..252: mol_type = other DNA
gtatccaggtgtggactcagccagggagggtggtgctggaggagccacctccctgtctctctggccaaaggcccgctctacaaggtctcccggggacacctggccgggaccagtgggcagccctgcccgtgcccaagagggcactcagagaatgggcacgtgcttggtggcacacacgtggcagggctggcgggctgtgtcgggaatgtatttataaacgctgtcttcagagcaaattccattctattctaa

SEQ ID NO: 163 - protein, 83 aa, synthetic construct
VSRCGLSQGGWCWRSHLPVSLAKGPLYKVSRGHLAGTSGQPCPCPRGHSENGHVLGGTHVAGLAGCVGNVFINAVFRANSILF

SEQ ID NO: 164 - DNA, 456 bp, synthetic construct
  source 1..456: mol_type = other DNA
cctctggcctgttccctggagccctggtcagcacccccctgcacccccagctccccttccctctggggttttgtctctttgtcactttgtaatccttgcccagactgctatctacgggggacagcatttcctgcctttgtttcctctcccagttgggcccctggctccctctcaaaagcattccccgggccctttcaaacccgcctaggccgggggctgatgatgcaggcaggagggggccccagctgggcccacctattgttcaccaggccccccacccgatgtctcccacacccccaccccatgcccgactggccagccctggccaacacaatggggcaacttccaaatttagcttttctgctgtttctttccaaggtccttcgcccccaccctcatattgcccctccacaccccgggtgggggtcgggtcggagaagacgaggttttcaatag

SEQ ID NO: 165 - protein, 151 aa, synthetic construct
PLACSLEPWSAPPCTPSSPSLWGFVSLSLCNPCPDCYLRGTAFPAFVSSPSWAPGSLSKAFPGPFQTRLGRGLMMQAGGGPSWAHLLFTRPPTRCLPHPHPMPDWPALANTMGQLPNLAFLLFLSKVLRPHPHIAPPHPGWGSGRRRRGFQ

SEQ ID NO: 166 - DNA, 303 bp, synthetic construct
  source 1..303: mol_type = other DNA
accattgtcaggggctccacaggggctgtgtccagggcatgctggtcccccctggggactatgggaatttctccattcagcacttcctatgggaacgctgggtggaggggcactggaaagtggcctcagagctctgggtccttgccctgccctggaggccgaggagggttcgcttacagtagcaaaagggaacggttatttttaactccattgacatgggttctgtccaaaaatgtggctgaagagcccagagtggggctgaaggccctccgagggtacagtctgggccccatcacctcctga

SEQ ID NO: 167 - protein, 100 aa, synthetic construct
TIVRGSTGAVSRACWSPLGTMGISPFSTSYGNAGWRGTGKWPQSSGSLPCPGGRGGFAYSSKRERLFLTPLTWVLSKNVAEEPRVGLKALRGYSLGPITS

SEQ ID NO: 168 - DNA, 225 bp, synthetic construct
  source 1..225: mol_type = other DNA
gagcaggggctgcccctctggggctggcactcacggaccaggctcttgaatgcgtcaaaaggcattcctggcagggtgtgggctcagtccagagaaggcgctctcaggaagctctccggacaggtgtgcggaggctgcccaagaatcctctatggcctcccaagccactgtgacaaaaagtcacaggcagacctccagacaggctgggtatgggacattaagtaa

SEQ ID NO: 169 - protein, 74 aa, synthetic construct
EQGLPLWGWHSRTRLLNASKGIPGRVWAQSREGALRKLSGQVCGGCPRILYGLPSHCDKKSQADLQTGWVWDIK

SEQ ID NO: 170 - DNA, 234 bp, synthetic construct
  source 1..234: mol_type = other DNA
tggaaaagctacaagaagggcagagggcaaggtcctgctattgttttggccgcagtgtctgcactgctgctcttcaggctttcgaggaaagattccccacagaggacgctggggtgggaagagaaggcaggcagctacctcagcccctgcccaagtggtcttacagaggcacttgggtggttctgtcctccaggtgaggaagatcgagattgtacggaagaagccaagctttaa

SEQ ID NO: 171 - protein, 77 aa, synthetic construct
WKSYKKGRGQGPAIVLAAVSALLLFRLSRKDSPQRTLGWEEKAGSYLSPCPSGLTEALGWFCPPGEEDRDCTEEAKL

SEQ ID NO: 172 - DNA, 228 bp, synthetic construct
  source 1..228: mol_type = other DNA
ggatgtagcccccagttggccctttggtcttgctgccaaccaatcccccctcactgtgacaccccagccagcctggcctttttgaatggccagctacatttctgcctcagggcctttgcacatgccactctgtctgaaactcacttctctcagctcttcacaagcctactccttctcttcatttggatcttagctcagaagtcatctcctcctagaagtctgccctga

SEQ ID NO: 173 - protein, 75 aa, synthetic construct
GCSPQLALWSCCQPIPPHCDTPASLAFLNGQLHFCLRAFAHATLSETHFSQLFTSLLLLFIWILAQKSSPPRSLP

SEQ ID NO: 174 - DNA, 246 bp, synthetic construct
  source 1..246: mol_type = other DNA
gggtttctggctattttcatatactatctcctaatcctaggaggccagggctgctggcatctccattttagagatgtggaaattgaggcacagggagtttatatgacttgcccaaaccacatgactaacacgtgggagagcccagatttgaacccaggtggtctggcccaccatctgagctctggactgccccactgtgccgttactctaagtggcgagggtaaggcagacgtcaggcgcaactga

SEQ ID NO: 175 - protein, 81 aa, synthetic construct
GFLAIFIYYLLILGGQGCWHLHFRDVEIEAQGVYMTCPNHMTNTWESPDLNPGGLAHHLSSGLPHCAVTLSGEGKADVRRN

SEQ ID NO: 176 - DNA, 834 bp, synthetic construct
  source 1..834: mol_type = other DNA
ttcggacggccaatggtgcttccccgcccctccacgcgtccgtccacccctctgccagtgggtctcccctcagtggcscadmctcaggggctgcgaggagcgctacgcgcctggtcccgtcccgcctcagctcggcggccgccgggagcccgcaccgagccggctcctgggagggccggcccctctcgggcctccaacgaggagcaggaaggaggcggcggcggcggcgaaggggttaaggtgaagggcttcgaggccgcggccgggccttgggccgcagccagcgcaggttgttttgaccacggaggagccgtctccgtctccttttgttctcggggctcctcgagggccgccggccgtccgccctggggccccgcccttccgcggccgtcccccgtggcccgcacccgggagggaggacgcggggatcagcctggctgcctgcagtcccctcccgacgccccctcctctcctcctgctgatgccccccgggccgcggccagctgttggggcggggggcgccggccggccccagctgccgcctcgccgcggggcctgggggctgggccctgtgccagggcgtcctgggaacggcggcgccccagccgctgctctccgcagcccaccccgcccggccccccgactcgctcactcaccccacgcatgcacactcttggccggaggcgatgctgcgctccggcgggcgggcgcgcagggcgacgggcacgcactggcgcggccgggtcgcgcgcccgccgccacgcccgtgcacatgcgggacacacgcgcgcgcactacacacacacacgcatggtccccgcacacacggcttga

SEQ ID NO: 177 - protein, 277 aa, synthetic construct
FGRPMVLPRPSTRPSTPLPVGLPSVAXXQGLRGALRAWSRPASARRPPGARTEPAPGRAGPSRASNEEQEGGGGGGEGVKVKGFEAAAGPWAAASAGCFDHGGAVSVSFCSRGSSRAAGRPPWGPALPRPSPVARTREGGRGDQPGCLQSPPDAPSSPPADAPRAAASCWGGGRRPAPAAASPRGLGAGPCARASWERRRPSRCSPQPTPPGPPTRSLTPRMHTLGRRRCCAPAGGRAGRRARTGAAGSRARRHARAHAGHTRAHYTHTRMVPAHTA

SEQ ID NO: 178 - DNA, 318 bp, synthetic construct; identical to SEQ ID NO: 72
  source 1..318: mol_type = other DNA

SEQ ID NO: 179 - protein, 105 aa, synthetic construct; identical to SEQ ID NO: 73

SEQ ID NO: 180 - DNA, 243 bp, synthetic construct; identical to SEQ ID NO: 74
  source 1..243: mol_type = other DNA

SEQ ID NO: 181 - protein, 80 aa, synthetic construct; identical to SEQ ID NO: 75

SEQ ID NO: 182 - DNA, 420 bp, synthetic construct; identical to SEQ ID NO: 76
  source 1..420: mol_type = other DNA

SEQ ID NO: 183 - protein, 139 aa, synthetic construct; identical to SEQ ID NO: 77

SEQ ID NO: 184 - DNA, 243 bp, synthetic construct; identical to SEQ ID NO: 78
  source 1..243: mol_type = other DNA

SEQ ID NO: 185 - protein, 80 aa, synthetic construct; identical to SEQ ID NO: 79

SEQ ID NO: 186 - DNA, 279 bp, synthetic construct; identical to SEQ ID NO: 80
  source 1..279: mol_type = other DNA

SEQ ID NO: 187 - protein, 92 aa, synthetic construct; identical to SEQ ID NO: 81

SEQ ID NO: 188 - DNA, 279 bp, synthetic construct; identical to SEQ ID NO: 82
  source 1..279: mol_type = other DNA

SEQ ID NO: 189 - protein, 92 aa, synthetic construct; identical to SEQ ID NO: 83

SEQ ID NO: 190 - DNA, 243 bp, synthetic construct; identical to SEQ ID NO: 84
  source 1..243: mol_type = other DNA

SEQ ID NO: 191 - protein, 80 aa, synthetic construct; identical to SEQ ID NO: 85

SEQ ID NO: 192 - DNA, 474 bp, synthetic construct; identical to SEQ ID NO: 86
  source 1..474: mol_type = other DNA

SEQ ID NO: 193 - protein, 157 aa, synthetic construct; identical to SEQ ID NO: 87

SEQ ID NO: 194 - DNA, 237 bp, synthetic construct; identical to SEQ ID NO: 88
  source 1..237: mol_type = other DNA

SEQ ID NO: 195 - protein, 78 aa, synthetic construct; identical to SEQ ID NO: 89

SEQ ID NO: 196 - DNA, 312 bp, synthetic construct; identical to SEQ ID NO: 90
  source 1..312: mol_type = other DNA

SEQ ID NO: 197 - protein, 103 aa, synthetic construct; identical to SEQ ID NO: 91

SEQ ID NO: 198 - DNA, 315 bp, synthetic construct; identical to SEQ ID NO: 92
  source 1..315: mol_type = other DNA

SEQ ID NO: 199 - protein, 104 aa, synthetic construct; identical to SEQ ID NO: 93

SEQ ID NO: 200 - DNA, 231 bp, synthetic construct; identical to SEQ ID NO: 94
  source 1..231: mol_type = other DNA

SEQ ID NO: 201 - protein, 76 aa, synthetic construct; identical to SEQ ID NO: 95

SEQ ID NO: 202 - DNA, 435 bp, synthetic construct; identical to SEQ ID NO: 96
  source 1..435: mol_type = other DNA

SEQ ID NO: 203 - protein, 144 aa, synthetic construct; identical to SEQ ID NO: 97

SEQ ID NO: 204 - DNA, 273 bp, synthetic construct; identical to SEQ ID NO: 98
  source 1..273: mol_type = other DNA

SEQ ID NO: 205 - protein, 90 aa, synthetic construct; identical to SEQ ID NO: 99

SEQ ID NO: 206 - DNA, 555 bp, synthetic construct; identical to SEQ ID NO: 100
  source 1..555: mol_type = other DNA

SEQ ID NO: 207 - protein, 184 aa, synthetic construct; identical to SEQ ID NO: 101

SEQ ID NO: 208 - DNA, 1644 bp, synthetic construct
  source 1..1644: mol_type = other DNA
tattgttcccctcgtccgtctgtctcgatgcctgattcggacggccaatggtgcttccccgcccctccacgcgtccgtccacccctctgccagtgggtctcccctcagtggcscadmatctgtggccagcctaattcaagaaagtcgtttggaagctcgaaaatattacggaaaggagccagatttgattgttgttccttttacaaaaatgcagattcaaggcttgatgcagtttacagttttcccatcgccttggctcattttacaggaactttagataatcattatcctaagcataaattgcttcagttttttcaacatcatgatccaatttttccttcaattgtgtcacatgctcctcttcctgctgttccaaatgtttttactgatggatctaataatggagtagctgtttatgcactcaataaaaaagtcaccaagagagtacagacacctccagcttcagctcaaatagttgagcttcgagcagtacataaggtgctgcttgattttgcttctcagtcttttaatttattctctgacagccattatgtggttcgtgcagtcagaaatttagaaacagtaccttttattagcactagtaatcctgttattcaggatttgtttcttcagatacaacaggccattcagctgcgctgtaaaaaattttatattggccatattagagctcactctaatcttccaggtcctttagcagcaggcaatcaaattgcagattctgccacgcagcttattgccttaactcaaatagaaaaagcacaaaaggctcatagcctccaccatcaaaatagccagagcctaagattacagtataagatcctcagagaagcagcacgccagattataaaacaatgtccagattgctcgcatttacaacctgtgcctcattatggcattaaccctcgaggcttgcgtcccaatgatctgtggcaaatggatgttactcatatacctgaatttggaaaattaaaatacgtccatgtctctatagacacgttttctggctttgtaatagcttctgctcaatcaggagaagctacatctcatgttattagacattgtcttgctgcttttgccatgattggcactcctaaaaaacttaaaacagataatggctccggctacaccagtaaaaaatttgctttattttgtcaacaatttttaattaatcatgttactggcattccttacaatccccagcgacaagggattgttgaacgtactcatggcacattaaaagtcattttacaaaaaataaaaaagggggagttatatcccctaacgccccataattacttgtctcattctctttttattcaaaattttttgaccttggatgcccatggtaagagtgctgcagagcgcttttggcatccttctactgccactcaggctttggtcaaatggaaagatccacttactggatcttggcaaggcccagatccagtcctcatatggggccgagggcatgtttgtgtttttccacaggatgcagaaggccctcggtggctgccagaacgattggtgcgacatgtggaccctctacctgctgatgacattgatgascadmatgttgttttatgtgtccatcagaaggcaatcctagggcgtgagtgtcattga

SEQ ID NO: 209 - protein, 547 aa, synthetic construct
YCSPRPSVSMPDSDGQWCFPAPPRVRPPLCQWVSPQWXXICGQPNSRKSFGSSKILRKGARFDCCSFYKNADSRLDAVYSFPIALAHFTGTLDNHYPKHKLLQFFQHHDPIFPSIVSHAPLPAVPNVFTDGSNNGVAVYALNKKVTKRVQTPPASAQIVELRAVHKVLLDFASQSFNLFSDSHYVVRAVRNLETVPFISTSNPVIQDLFLQIQQAIQLRCKKFYIGHIRAHSNLPGPLAAGNQIADSATQLIALTQIEKAQKAHSLHHQNSQSLRLQYKILREAARQIIKQCPDCSHLQPVPHYGINPRGLRPNDLWQMDVTHIPEFGKLKYVHVSIDTFSGFVIASAQSGEATSHVIRHCLAAFAMIGTPKKLKTDNGSGYTSKKFALFCQQFLINHVTGIPYNPQRQGIVERTHGTLKVILQKIKKGELYPLTPHNYLSHSLFIQNFLTLDAHGKSAAERFWHPSTATQALVKWKDPLTGSWQGPDPVLIWGRGHVCVFPQDAEGPRWLPERLVRHVDPLPADDIDXXXVVLCVHQKAILGRECH

SEQ ID NO: 210 - DNA, 399 bp, synthetic construct
  source 1..399: mol_type = other DNA
tgtgggacacagctccctagcccatgctggtattatgagccttgcgctccccctaattcctcccccatcactggtcgttggtcgactgctcacagcagctcacggcagctcttgccggctgccagccactcatgctggttgccggccactcacgctgaccatcggccgctcctggcagcacacggcagcacacagcagcccgcggcagctcacgccgacctctggctgctcgcagcccagctccagggagccgttgttcacaatcttagctgtagagggtgcagctcactggcccatgtgggaatcgaaccggtgacctcgttgttaggcgcacggcgctccaaccacctgagccaccaggcggccctgattgtgtttctatatactgtgtttccctga

SEQ ID NO: 211 - protein, 132 aa, synthetic construct
CGTQLPSPCWYYEPCAPPNSSPITGRWSTAHSSSRQLLPAASHSCWLPATHADHRPLLAAHGSTQQPAAAHADLWLLAAQLQGAVVHNLSCRGCSSLAHVGIEPVTSLLGARRSNHLSHQAALIVFLYTVFP

SEQ ID NO: 212 - DNA, 246 bp, synthetic construct
  source 1..246: mol_type = other DNA
aataagaccgggttttatattaagttttgctccaaaagacgcattagagctgattgtccagctaggtcttattttcggggaaacatggtagagaatcatacagattctctgcatataaggaattttgtaaaggagaagggtactgagcagagattatatctctcaaataacactattctctcttcctttttgattttacagtggaggaaaggaggacaaagtactaaagtgaaaagtagatcttga

SEQ ID NO: 213 - protein, 81 aa, synthetic construct
NKTGFYIKFCSKRRIRADCPARSYFRGNMVENHTDSLHIRNFVKEKGTEQRLYLSNNTILSSFLILQWRKGGQSTKVKSRS

SEQ ID NO: 214 - DNA, 240 bp, synthetic construct
  source 1..240: mol_type = other DNA
agagaactgaggttgcttgtctttatagctactagtggcctcaaaaggccaatacatctgtctccatttgtcccttgctcaataccctctgatttacaaagcctttcttctcttaggaaacgaatggcagagaatgaactgagccggtcggtgaatgagtttctgtccaagctgcaggatgacctcaaagaggcaatgaataccatgatgtgcagccgatgccagggaaagcataggtag

SEQ ID NO: 215 - protein, 79 aa, synthetic construct
RELRLLVFIATSGLKRPIHLSPFVPCSIPSDLQSLSSLRKRMAENELSRSVNEFLSKLQDDLKEAMNTMMCSRCQGKHR

SEQ ID NO: 216 - DNA, 393 bp, synthetic construct
  source 1..393: mol_type = other DNA
ggagttgtccttttgttgggttgtaggaggtttgaaatggaccgggaacctaagagtgccagatactgtgctgagtgtaataggctgcatcccgctgaagaaggagacttttgggcagagtctagcatgttgggcctgaaaatcacctactttgcgctgatggatggaaaggtgtatgacatcacaggtacacttctgtcctctagaattccagactcatgtatgctcaaaactgttatgtattggctaattatttctcatgcttgcagagtgggctggatgccagcgtgtgggaatctccccagatacccacagagtcccctatcacatctcatttggttctcggatcccaggcaccagtgggcgacagaggtgggtgatattttccaataa

SEQ ID NO: 217 - protein, 130 aa, synthetic construct
GVVLLLGCRRFEMDREPKSARYCAECNRLHPAEEGDFWAESSMLGLKITYFALMDGKVYDITGTLLSSRIPDSCMLKTVMYWLIISHACRVGWMPACGNLPRYPQSPLSHLIWFSDPRHQWATEVGDIFQ

SEQ ID NO: 218 - DNA, 255 bp, synthetic construct
  source 1..255: mol_type = other DNA
ctcttaatttcttttgcctcattattcttttgttttccacccagagccaccccagatgcccctcctgctgaccttcaggatttcttgagccggatctttcaagtacccccaggacagatgtctaatgggaacttctttgcagctcctcagcctggccctgggggcaccgcagcctccaagcctaacagcacagtacccaagggagaagccaaaccgaagaggcggaagaaagtgaggaggcccttccaacgttga

SEQ ID NO: 219 - protein, 84 aa, synthetic construct
LLISFASLFFCFPPRATPDAPPADLQDFLSRIFQVPPGQMSNGNFFAAPQPGPGGTAASKPNSTVPKGEAKPKRRKKVRRPFQR

SEQ ID NO: 220 - DNA, 315 bp, synthetic construct
  source 1..315: mol_type = other DNA
caccccttctcttctctcctcaaatcaatgtcagggagtcaaaagggctgtgtacagcacaggatggagtttgatttgtttatttttaaatatttaaaaaggaaaattttaagctcaaattgttcactcagtacttgtagscadmgagaacaggtctggggtattttccccaggggtcatagatttacctgtactccaccaaaaaactgcaaaggcaataatttggaaaacagatacacctgtgtgaatagatcagtggccccttacacagaaaaagatatcggccgcccaggcgcttgtacaggagcagcttga

SEQ ID NO: 221 - protein, 104 aa, synthetic construct
HPFSSLLKSMSGSQKGCVQHRMEFDLFIFKYLKRKILSSNCSLSTCXXREQVWGIFPRGHRFTCTPPKNCKGNNLENRYTCVNRSVAPYTEKDIGRPGACTGAA

SEQ ID NO: 222 - DNA, 237 bp, synthetic construct
  source 1..237: mol_type = other DNA
gtgatagctccacgacctcgtgttacggagcttgagtgggctcgtaactgcgtttccggcactgtcttacggctaaacggcgatcaaaacttcggttttgccagggcgggggtttataccgccacgcttaattgccacgatagtcttggtcccgcgaggggcacggccagccgagcatctgtgtgttttacttgtgtgaaagaagggccgaggataaagggaaatgggtcacgctaa

SEQ ID NO: 223 - protein, 78 aa, synthetic construct
VIAPRPRVTELEWARNCVSGTVLRLNGDQNFGFARAGVYTATLNCHDSLGPARGTASRASVCFTCVKEGPRIKGNGSR

SEQ ID NO: 224 - DNA, 981 bp, synthetic construct
  source 1..981: mol_type = other DNA
caaagacggcaaacccttacagggaaactgggtgaggggccagccccaggccccgactcagcaatgttatggggcactgcaggttcaggaacagacccaggagccgaaaaagaacgaacccctgctaggaagcatgtcacagacttattcagggccaccacaggcagcgcaggattggacttgtgttccacctccgacatcatattaactcctgaaatgggaatgcaagttttgcccactggagtttttgggcccctgccacctaaaacggtgggtttactgttaggaagaagcagctccgttataaagggaattcatgtttctccagggattattgatgaggattttacaggagaaataaaaattatggctcattctcctcttaatatttctgccattcctgctggaacccgtattgcacaactgtttattttgcctcgtcttaatattggaaaaaacaggcaaaatcaagagcgggggaaccaaggatttggctcttctgatgtatattggattcaagaaataaaaaaggatcgacccgtattgttactcaaaataaatggaaaagattttcaaggacttctggacactggagccgatgtctcgtgcatatctgctgaacattggccctccagttggccgacgcgctttactaataccaatttacaaggcataggccaatcgcaatcccccctccaaagtagtgatcttttgtcttggcaagatccggagggtcatcaggggacgtttcagccatatattatccctggtcttccagttaatttatggggaagagatgttatgagtaaaatgggagtttatctttacagtcctagttcacaagtaactcaacagatgtttgatcaaggctttctccctggtcagggcttaggctcggtgggacaagggcgccgagagcctatttcaactaatcctaacttacagagaacaggtctggggtattttccccaggggtcatag

SEQ ID NO: 225 - protein, 326 aa, synthetic construct
QRRQTLTGKLGEGPAPGPDSAMLWGTAGSGTDPGAEKERTPARKHVTDLFRATTGSAGLDLCSTSDIILTPEMGMQVLPTGVFGPLPPKTVGLLLGRSSSVIKGIHVSPGIIDEDFTGEIKIMAHSPLNISAIPAGTRIAQLFILPRLNIGKNRQNQERGNQGFGSSDVYWIQEIKKDRPVLLLKINGKDFQGLLDTGADVSCISAEHWPSSWPTRFTNTNLQGIGQSQSPLQSSDLLSWQDPEGHQGTFQPYIIPGLPVNLWGRDVMSKMGVYLYSPSSQVTQQMFDQGFLPGQGLGSVGQGRREPISTNPNLQRTGLGYFPQGS

SEQ ID NO: 226 - DNA, 321 bp, synthetic construct
  source 1..321: mol_type = other DNA
ctttggatgcctatgttaagagtgcagctgaacgtttctggcatccttctgccgtccctgaggctttggtcagaaagaaggatccacttactggatcatggcaaggcccagacccagtcctcatatggggccgagggcatgtttgtgtttttccacaggatgcagatagtcctcggtggctgccagaacgattggtgcgacatgtggaccctctacctgctgatgacattgatgaccctcagcaatgcagaagaagaccagacgtattgggcctacgtacctgatccacctattctccaccctgctgtatscadmatgtaa

SEQ ID NO: 227 - protein, 106 aa, synthetic construct
LWMPMLRVQLNVSGILLPSLRLWSERRIHLLDHGKAQTQSSYGAEGMFVFFHRMQIVLGGCQNDWCDMWTLYLLMTLMTLSNAEEDQTYWAYVPDPPILHPAVXXM

SEQ ID NO: 228 - DNA, 234 bp, synthetic construct
  source 1..234: mol_type = other DNA
ggcgtgagtgtcactgacataatctggaatctcaggaccatcccatacagcagggtggagaataggtggatcaggtacgtaggcccaatacgtctggtcttcttctgcattgctgagggtcatcaatgtcatcagcaggtagagggtccacatgtcgcaccaatcgttctggcagccaccgaggactatctgcatcctgtggaaaaacacaaacatgccctcggccccatatga

SEQ ID NO: 229 - protein, 77 aa, synthetic construct
GVSVTDIIWNLRTIPYSRVENRWIRYVGPIRLVFFCIAEGHQCHQQVEGPHVAPIVLAATEDYLHPVEKHKHALGPI

SEQ ID NO: 230 - DNA, 231 bp, synthetic construct
  source 1..231: mol_type = other DNA
gcggtgaggacgtgtgcgcccttcctccttcctctttctcgactccatcttcgcggtagcggtagcggccgcagttcaggtaagatttgggccacggctggatccggacgacttaataggttagccgcgaggtctgacggcttgggaaaaatagaggaagaggggctgctctgtgggccgggttcttgtcaccacccgacctccctggctggcctggccttaggcacgtga

SEQ ID NO: 231 - protein, 76 aa, synthetic construct
AVRTCAPFLLPLSRLHLRGSGSGRSSGKIWATAGSGRLNRLAARSDGLGKIEEEGLLCGPGSCHHPTSLAGLALGT

SEQ ID NO: 232 - DNA, 261 bp, synthetic construct
  source 1..261: mol_type = other DNA
gacccgcgatcgtccccggcccgccacccactccccgactcccttactcccagagcatttcttctcttacaagcatttctttcctcagtcgccgacatgcagctctttgttcgcgcccaagatctacacaccctcgaggtgaccggccaggagactgtctcccagatcaaggtaaggctgcgtggtgctcctggtctgcatcctcttgtgttctttaacctcgctccccacgggagcgctgagcctcactttcccctgtag

SEQ ID NO: 233 - protein, 86 aa, synthetic construct
DPRSSPARHPLPDSLTPRAFLLLQAFLSSVADMQLFVRAQDLHTLEVTGQETVSQIKVRLRGAPGLHPLVFFNLAPHGSAEPHFPL

SEQ ID NO: 234 - DNA, 300 bp, synthetic construct
  source 1..300: mol_type = other DNA
agtccatggttccttggcccgtgctgggaaagtaagaggtcagactcccaaggtaagagagtattagtggtgccctttggacttttgttttcctgtcaccttcctcatgaaatgagcctgagggaaggcacggaagagatgaaccagggtctgattagccctcctttttcccaggtggccaaacaggagaagaagaagaagaagactggccgagccaagcggcggatgcagtacaaccggcgttttgtcaatgttgtgcccacctttggcaagaagaagggccccaatgccaactcttaa

SEQ ID NO: 235 - protein, 99 aa, synthetic construct
SPWFLGPCWESKRSDSQGKRVLVVPFGLLFSCHLPHEMSLREGTEEMNQGLISPPFSQVAKQEKKKKKTGRAKRRMQYNRRFVNVVPTFGKKKGPNANS

SEQ ID NO: 236 - DNA, 309 bp, synthetic construct
  source 1..309: mol_type = other DNA
ccttggatgcccatggtaagagtgctgcagagcgcttttggcatccttccactgccactcaggctttgttcaaatggaaagacccacttacaggctcttggcaaggcccagatccagtcctcatatggggccgagggcatgtttgtgtttttccacaggatgcagaaggccctcggtggttgccagaacgattggtgcgacatgtggaccctctacctgctgatgacattgatgascadmttacaaaactttccaaatgttgttttatgtgtccatcagaaggcaatcctagggcgtgagtgtcattga

SEQ ID NO: 237 - protein, 102 aa, synthetic construct
PWMPMVRVLQSAFGILPLPLRLCSNGKTHLQALGKAQIQSSYGAEGMFVFFHRMQKALGGCQNDWCDMWTLYLLMTLMXXLQNFPNVVLCVHQKAILGRECH

SEQ ID NO: 238 - DNA, 333 bp, synthetic construct
  source 1..333: mol_type = other DNA
aggggaatgggacttaattggggaacagtgtgtacttccaggacattttccaagtcaagttgtcctttcagtcttagttgtggagggcactgttcagccccaggtccagttgccgttgttagttgcagggggtggagcccagcaccccttgcgggagttgaaccagcaagcttgtggttgagagcccactggcccatgtgggctctggaaccggcagccttcaatgttaggagcacagagctccaaccgcctgagccactgggccggcccaccccccctttttttttttttaagaaaaagtatttttttctctcaaaagcttccttatattag

SEQ ID NO: 239 - protein, 110 aa, synthetic construct
RGMGLNWGTVCTSRTFSKSSCPFSLSCGGHCSAPGPVAVVSCRGWSPAPLAGVEPASLWLRAHWPMWALEPAAFNVRSTELQPPEPLGRPTPPFFFFKKKYFFLSKASLY

SEQ ID NO: 240 - DNA, 612 bp, synthetic construct
  source 1..612: mol_type = other DNA
gggagaatgaatgaattagcctttgaagctgatgtgtctgatttggttcttttcctctcaggtgaaaagctccgggtcttaggctacaatcacaatggcgaatggtgtgaagcccaaaccaaaaatggccaagggtgggttcccagcaactacatcacgcccgtcaacagcctggagaaacattcctggtaccacgggcccgtgtcccgcaatgctgccgagtacctgctgagcagtgggatcaacggcagcttcctggtgcgggagagtgagagcagccccgggcagaggtccatctcgctgagatacgaagggagggtgtaccactacaggatcaacacagcttcggacggcaaggtgggcggggcggggcgccgggggcggggcctgagtcttgggccagaactcagagatccctctgctgggtggataatgtttttacgacaatactcgagaagtggttggcagacactttcatgtaaacagcaggcgtcattcattagcctcatcgatgatcccctgtggaggactgatcatgtgacattacaagtccacgggctgggctggttctctggttgtcctgctggacgtttgttgttaacagtttcataa

SEQ ID NO: 241 - protein, 203 aa, synthetic construct
GRMNELAFEADVSDLVLFLSGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKVGGAGRRGRGLSLGPELRDPSAGWIMFLRQYSRSGWQTLSCKQQASFISLIDDPLWRTDHVTLQVHGLGWFSGCPAGRLLLTVS

SEQ ID NO: 242 - DNA, 273 bp, synthetic construct
  source 1..273: mol_type = other DNA
gggtgtagccagatggattgtcggtgtggctccagatggtgtatatattttttagtaatatgtaatgtatgcacacggtttttaaaaaaatcaattacagtgaaaggtaatttcgtttctagtttagttccctgcccagaagcaatcactgtaaccaccttcttccagagtaacacggtgttatatacacggtgtatatactgtgtttccctgaaaataagacctaaccggacagtaagccctagcatgatttttcaggatgacgtcccctga

SEQ ID NO: 243 - protein, 90 aa, synthetic construct
GCSQMDCRCGSRWCIYFLVICNVCTRFLKKSITVKGNFVSSLVPCPEAITVTTFFQSNTVLYTRCIYCVSLKIRPNRTVSPSMIFQDDVP

SEQ ID NO: 244 - DNA, 462 bp, synthetic construct
  source 1..462: mol_type = other DNA
cttgtagaatttgagagtcagccaatgaggaagccgacccctctgtctaaaagctggtgtgtgctggggctcctttcactccgggtggaactcagggagttcatttgctcaagcactgtccacccccgggcagctcgtcagacagttctgggcttctcgccctcctccctccctccctccagctgtctgagcacctggagcctcctgggcctacagggtcatcgggcagaccctctgcagaggctcctgcctgtgttgggtgggagcacattccaaaaggagtggaacagtgtctgcatggggaggtactccagtgatgcaggcgacagcctggcactgaggagctgctccaagcggagctttgaggggatccttttaggatttctaaggggaacatttaaggctggtaggagggacaggctggggttgaagaaatttagttcttattttcaaatgagctga

SEQ ID NO: 245 - protein, 153 aa, synthetic construct
LVEFESQPMRKPTPLSKSWCVLGLLSLRVELREFICSSTVHPRAARQTVLGFSPSSLPPSSCLSTWSLLGLQGHRADPLQRLLPVLGGSTFQKEWNSVCMGRYSSDAGDSLALRSCSKRSFEGILLGFLRGTFKAGRRDRLGLKKFSSYFQMS

SEQ ID NO: 246 - DNA, 237 bp, synthetic construct
  source 1..237: mol_type = other DNA
ggtagtaggatcgctacgaaaagactgtcagttataaaacctctgagccagagtttgctattggcttgcctgacttttaactgtccatgtgtgtcatctccccagaacagagagagagagagagagagagagagagagagaaagagagagagaatctccttgttaatgaatcctgcttaccttcttgagggttatagaaggtatcaacttgtatatgttgttatttctctcttttaa

SEQ ID NO: 247 - protein, 78 aa, synthetic construct
GSRIATKRLSVIKPLSQSLLLACLTFNCPCVSSPQNRERERERERERKRERISLLMNPAYLLEGYRRYQLVYVVISLF

SEQ ID NO: 248 - DNA, 318 bp, synthetic construct
  source 1..318: mol_type = other DNA
aaagagaagaaaaatgatagctgtccccatccacattgcgccctctgtcgtgtgctcctttcccttctctcgtctcagttggtccggacgagaactccttgtggaggggcttcctgcacaggtgctcaccactgtccatctcacaggagactcatgtgcgtgtgtctgaaaaccctcttcctgccttcccggccatggaaaaacctggatggccttgggcagccctccagcccctgctctgttcctggagagcactggccaaggaaccacggggtgtattactgggtcacggggtgtactgcaggtcttgatctatga

SEQ ID NO: 249 - protein, 105 aa, synthetic construct
KEKKNDSCPHPHCALCRVLLSLLSSQLVRTRTPCGGASCTGAHHCPSHRRLMCVCLKTLFLPSRPWKNLDGLGQPSSPCSVPGEHWPRNHGVYYWVTGCTAGLDL

SEQ ID NO: 250 - DNA, 288 bp, synthetic construct
  source 1..288: mol_type = other DNA
ggcttcgctgtgcatcgcgtttcgttagcagcaaagctggttcgttggcgttgtttgcgttggtgtctgctctgtggcctgaaggctgtccctgttttcctcagctctacgtctcctcagagagccgcttcaacactttggccgagttggttcatcatcactccactgtggcagacgggctcatcaccactctccactatccagcccccaagcgcaacaagcccaccgtctacggcgtgtctcccaactatgacaagtgggagatggagcgcacggacatcaccatga

SEQ ID NO: 251 - protein, 95 aa, synthetic construct
GFAVHRVSLAAKLVRWRCLRWCLLCGLKAVPVFLSSTSPQRAASTLWPSWFIITPLWQTGSSPLSTIQPPSATSPPSTACLPTMTSGRWSARTSP

SEQ ID NO: 252 - DNA, 243 bp, synthetic construct
  source 1..243: mol_type = other DNA
ccaaaaagcgctcagctcttctgtggatttttgttggcagatttgaaatgcaagtgctgcttagttcctagcaggttcctgttctttgtattgtgtgtccagacttctggaatgaagcaaacattaaggcttcttactaactcagatcagcccttccccccttctttcttgttatctgtgacttgcaccctcgccactaatgcacagtgtttgtggtttccaggcgctttgtttttcttttga

SEQ ID NO: 253 - protein, 80 aa, synthetic construct
PKSAQLFCGFLLADLKCKCCLVPSRFLFFVLCVQTSGMKQTLRLLTNSDQPFPPSFLLSVTCTLATNAQCLWFPGALFFF

SEQ ID NO: 254 - DNA, 312 bp, synthetic construct
  source 1..312: mol_type = other DNA
aaatcacttcttcccctctccccttctccgccatttgcccccctcagagtctatagctgtgatctaccttgctcttcaagactccttgggaaacccgtgcagctccagctccagctttcgtttgctcagcggttctcaccaagcacctcttcacctctccatgccagtcctcactgggcacctgagtctcggtcccctcctgcctccctgtcctgcctgttttgccttgctggccccgcaaagggcagtgccagctcctccttagccagcagggggagcaaggccggacttttaaccgcgactccatattga

SEQ ID NO: 255 - protein, 103 aa, synthetic construct
KSLLPLSPSPPFAPLRVYSCDLPCSSRLLGKPVQLQLQLSFAQRFSPSTSSPLHASPHWAPESRSPPASLSCLFCLAGPAKGSASSSLASRGSKAGLLTATPY

SEQ ID NO: 256 - DNA, 645 bp, synthetic construct
  source 1..645: mol_type = other DNA
aagcacatggcagcatgctgtggacactggtctgtagcctactgtccactgactgtatccgcacagctgttccttgtcggtacacataaggtcgccttgtttttatgtggtggatgtcagcatgtagcagccctctgtgggcatttgcgttcttcccagtgcgtggctgttacagaagtgctgcagggattctccttgtttgcacacaggggacagtgtcctggagggccagcactcagaggggaacgactgcgtcaggggccgtgtgtgtttgtcgtcttcctcacactcccaaagcctcccaaggagctcgtacctgtctgcgctctgccgcgcgtgttgggggagtgcctgcttcccgtccctgcactgacacagtgtgctttgctttggggtttatttttgtcattttcccccaggaaatttattggcaagctcagaaacgagcagagaaggaaaggttccgtgacagcactgacactagaccggcccacgcagtggccatgtgactacgcggggggtgtgcaccagggagaggccaccattgccgtgtggcacttgctgttacactgggttctcttctggctgtgcagcgagacccagctgccgtgtttggggaccagacttctgggggctcctctgtga

SEQ ID NO: 257 - protein, 214 aa, synthetic construct
KHMAACCGHWSVAYCPLTVSAQLFLVGTHKVALFLCGGCQHVAALCGHLRSSQCVAVTEVLQGFSLFAHRGQCPGGPALRGERLRQGPCVFVVFLTLPKPPKELVPVCALPRVLGECLLPVPALTQCALLWGLFLSFSPRKFIGKLRNEQRRKGSVTALTLDRPTQWPCDYAGGVHQGEATIAVWHLLLHWVLFWLCSETQLPCLGTRLLGAPL

SEQ ID NO: 258 - DNA, 357 bp, synthetic construct
  source 1..357: mol_type = other DNA
cttgtcctctggaagtcttccctcagatccgcggccagcggcgaatgcggcaatcctgggcagttgtgccgtaagcacaccttagagcctggtcgccccgaggggcaggtcccacatttcaataaactcgataaagctttcttcttgggggaggctagttttcaagacgttcactccccatctcccatacagtctttctcttcagacaattcaaactccctgtggaaacttgaagggtgggctcttgcctccctggtgggcctttgtagccaagttctcacagcaaacagatcgtgtcatttaccgccacccgcttcctgttttgagggtcagttcagaggacagtgggtcctttaa

SEQ ID NO: 259 - protein, 118 aa, synthetic construct
LVLWKSSLRSAASGECGNPGQLCRKHTLEPGRPEGQVPHFNKLDKAFFLGEASFQDVHSPSPIQSFSSDNSNSLWKLEGWALASLVGLCSQVLTANRSCHLPPPASCFEGQFRGQWVL

SEQ ID NO: 260 - DNA, 255 bp, synthetic construct
  source 1..255: mol_type = other DNA
tttcactacctttttttcctacaggaggacaccatggaggtggaagagtttttgaaggaagctgcggtaatgaaagagatcaagcaccctaacctagtacagttacttggtgagtgcgaggagctcggaagggggggcctttgcattaaacccgctggggtgatccaggtgctgtcaaagaggagatggctgcctcgctacatgaattcttctcatttggacatctgttctctactaacattcagccctcggtaa

SEQ ID NO: 261 - protein, 84 aa, synthetic construct
FHYLFFLQEDTMEVEEFLKEAAVMKEIKHPNLVQLLGECEELGRGGLCIKPAGVIQVLSKRRWLPRYMNSSHLDICSLLTFSPR

SEQ ID NO: 262 - DNA, 306 bp, synthetic construct
  source 1..306: mol_type = other DNA
tttaggccatttgatgtgtgcctggcctttgcttctgaactcggtggcagcctcttcctgtttaagttcattggcttgagaggaagaaaagagcaggccatgtaccaccccctgtctccccccccagaaacatcatctcaagtcacaggtgcttggaaccgtcttagcactgagtccagggcttgggggcagagtcagatccatttcagaagccttttccttgaggtccagtcctttctgatgcctgtgctgtgtctcgttggcaggggtctgcacccgggagcccccgttctatataatcactga

SEQ ID NO: 263 - protein, 101 aa, synthetic construct
FRPFDVCLAFASELGGSLFLFKFIGLRGRKEQAMYHPLSPPPETSSQVTGAWNRLSTESRAWGQSQIHFRSLFLEVQSFLMPVLCLVGRGLHPGAPVLYNH

SEQ ID NO: 264 - DNA, 264 bp, synthetic construct
  source 1..264: mol_type = other DNA
ccggcaggaggtgaacgctgtggtgctgctgtacatggccacgcagatctcgtcagccatggagtacctggagaagaaaaacttcatccacaggtaggagcctgccgaggccgcctccccacagggccccggcacccttctgtaaaaggccccaccttgaggggtgaccgctcggcctctcccttcagtgctggcaacatgttaggtctgagacaagagcgcagcggtgggttccgacgtggccagctctgggtgtgtgtctag

SEQ ID NO: 265 - protein, 87 aa, synthetic construct
PAGGERCGAAVHGHADLVSHGVPGEEKLHPQVGACRGRLPTGPRHPSVKGPTLRGDRSASPFSAGNMLGLRQERSGGFRRGQLWVCV

SEQ ID NO: 266 - DNA, 480 bp, synthetic construct
  source 1..480: mol_type = other DNA
ggggcatactcagtgtttcatacaaggagtcgagtgctccttgttccgccgagcccagccggcgggcgccgtagtgacctcttccccggagcgggtggccctgccctgacacacggcaagagcggccagtgcatgggtttcggttttgtgctgcgtgttttttttctcccttctctttattatcatttcattctccacttaacttgctgtcaccggcctcggcaatgtttccacaattggcagaattgtgtagatgcggctctaagtgaagtgtctttgctgtttcaaagcccggagtgttgtgaccttcaggtgcgccacaattatcctggtcttcacattctttgctggtggaaatggcttcctagcagagtgacagcctatccagggcagagcctgtgggctttgccagagtcgttcatacaagacattctctctgccaccactgtgacctttcctgtccaattatctcgactatga

SEQ ID NO: 267 - protein, 159 aa, synthetic construct
GAYSVFHTRSRVLLVPPSPAGGRRSDLFPGAGGPALTHGKSGQCMGFGFVLRVFFLPSLYYHFILHLTCCHRPRQCFHNWQNCVDAALSEVSLLFQSPECCDLQVRHNYPGLHILCWWKWLPSRVTAYPGQSLWALPESFIQDILSATTVTFPVQLSRL

SEQ ID NO: 268 - DNA, 495 bp, synthetic construct
  source 1..495: mol_type = other DNA
aaaaaaaaaaggaaaccaacataccaacatgacagcattactgatggctgctgctttttgtgttgtttttgtgtgtgtgtgtgtatgtggttcttagaagtggaaaaggaactggggaaaaaaggcatgcgaggggttgcaagcactctgctgcaggccccagagctgcccaccaagacaagaacctccaggagagctgtggaacacaaagaccccaccgacgtgcccgagacaccccactccaagggcccgggagagcctggtatgtctgcaccccacccccactgcaggctcagggtcagtgcccttagggccagggtggcagacggggagcagtgcgcgcagcctgcacagaaaggcaggcaaactcccattagttgtccagcggtggagaaggttcttctctccctgcagcatcccaccctccctctgggaatcgttaggggccattggcttcagcaggtagttcagtctgatgggcagaggtgcttctga

SEQ ID NO: 269 - protein, 164 aa, synthetic construct
KKKRKPTYQHDSITDGCCFLCCFCVCVCMWFLEVEKELGKKGMRGVASTLLQAPELPTKTRTSRRAVEHKDPTDVPETPHSKGPGEPGMSAPHPHCRLRVSALRARVADGEQCAQPAQKGRQTPISCPAVEKVLLSLQHPTLPLGIVRGHWLQQVVQSDGQRCF

SEQ ID NO: 270 - DNA, 291 bp, synthetic construct
  source 1..291: mol_type = other DNA
aaactggaaaaggtcaccccttcttgtttcccaagcataatggcccagtgtcactgcactctgtgggatgtgtcccgttccctccaggtcacaccctgtagaaaccaccagttggctggtctgagaggcacaggttatgaccctttgctcggccgtgtcatagtttttactcacaagatagtgaggggactctgcagatataaaggaaaccagtgcaggggtgggggagacggggacgtcccggctttttgttctgctgtcttcaaggagagagacctaagctcttcctaa

SEQ ID NO: 271 - protein, 96 aa, synthetic construct
KLEKVTPSCFPSIMAQCHCTLWDVSRSLQVTPCRNHQLAGLRGTGYDPLLGRVIVFTHKIVRGLCRYKGNQCRGGGDGDVPAFCSAVFKERDLSSS

SEQ ID NO: 272 - DNA, 438 bp, synthetic construct
  source 1..438: mol_type = other DNA
gtgcggcggggggcggccgcggccagtgggggggggcgctggagttggggcggcagggccgagcggcccggggcggggagtcgctgtcctcgccgagcgcgcgggcgcacgggggcgcaggtgagccgcgggcggggcgctgcggctgggggctgggggcggcagggcggcttcgtgtgccactcggcctcggcaggccagctcttcgagctccgtgtccctggctctgtcctccttgggaccccacaagtgccctcaggaaggctgtggggttcccctgcgccgaggcccacccgtggccatgcgctaggaggtgtctcccacccgccggagtcccaaggacccctcccaagagctcgggcaccctgcggccatcacacccaacaggcgagtcggggtgtaggaagtccactgctcacaagggcaccccctcattaa

SEQ ID NO: 273 - protein, 145 aa, synthetic construct
VRRGAAAASGGGRWSWGGRAERPGAGSRCPRRARGRTGAQVSRGRGAAAGGWGRQGGFVCHSASAGQLFELRVPGSVLLGTPQVPSGRLWGSPAPRPTRGHALGGVSHPPESQGPLPRARAPCGHHTQQASRGVGSPLLTRAPPH

SEQ ID NO: 274 - DNA, 252 bp, synthetic construct
  source 1..252: mol_type = other DNA
acatcagaaattggagacaccccggatggatgggggccttggccccaaaccctttttctgtcccacctgtttccgtgcccctacacctcctgtgggtcttttcttgtctgtgagtctgtgcttaccagggggaaccctgggcccacagggcctcctcactcacctgccttgttttctcagaacttctcatggctgcaggccccatgggtttcccttagtttaacttatgtgggtcttctccttggagcgtaa

SEQ ID NO: 275 - protein, 83 aa, synthetic construct
TSEIGDTPDGWGPWPQTLFLSHLFPCPYTSCGSFLVCESVLTRGNPGPTGPPHSPALFSQNFSWLQAPWVSLSLTYVGLLLGA

SEQ ID NO: 276 - DNA, 321 bp, synthetic construct
  source 1..321: mol_type = other DNA
atacttgtcgaatgcaccgacatgcccagtggggcctggaacctgtcgtcggttggcactggcctgcctgggcacgctgctgtgtgctccaccgtggcaggacctgttcccttagggagggggactggtgacctcagcctgggcgcctccagttcgggctttctgcctactcagcaacttctaatttgggtgcgtggttgggagatgctctcagctgtcagtcctgcccttggggggccagcttcctgcctctcacagccattaagtgcagctggacgcaggacccctgtcccactcctgggctgcaggagccacaggtga

SEQ ID NO: 277 - protein, 106 aa, synthetic construct
ILVECTDMPSGAWNLSSVGTGLPGHAAVCSTVAGPVPLGRGTGDLSLGASSSGFLPTQQLLIWVRGWEMLSAVSPALGGPASCLSQPLSAAGRRTPVPLLGCRSHR

SEQ ID NO: 278 - DNA, 321 bp, synthetic construct
  source 1..321: mol_type = other DNA
cacaagcttttctgcctgttgcaccgagggggaccctcgtcctcggacctgagggcacaagaggtgcagggaggggctcgtggtgcacatactgcgtcccaggaggggtgggggtccctaagcagtgtcctcgcgcaggactcctaccggaagcaagtggtcatcgatggggagacgtgtctgctggacatcctggacacggcgggccaggaggagtacagcgccatgcgggaccagtacatgcgcaccggcgagggtttcctctgcgtgtttgccatcaacaacaccaagtcctttgaagacatccaccagtaccggtga

SEQ ID NO: 279 - protein, 106 aa, synthetic construct
HKLFCLLHRGGPSSSDLRAQEVQGGARGAHTASQEGWGSLSSVLAQDSYRKQVVIDGETCLLDILDTAGQEEYSAMRDQYMRTGEGFLCVFAINNTKSFEDIHQYR

SEQ ID NO: 280 - DNA, 225 bp, synthetic construct
  source 1..225: mol_type = other DNA
gctggctccctgcccacctgtagccagggccccgcccgccccgccagggagccgtgctcaccgcccctctccctcgacacagggcagccgctctggctccagctccgggaccccgggacccagcggcccctcgcgctgtscadmcggagcccatgcgccggaggagctgcgcgccccggcccccgcccccgcccgacccggcccggggggctgtcgctccagtga

SEQ ID NO: 281 - protein, 74 aa, synthetic construct
AGSLPTCSQGPARPAREPCSPPLSLDTGQPLWLQLRDPGTQRPLALXXRSPCAGGAARPGPRPRPTRPGGLSLQ

SEQ ID NO: 282 - DNA, 558 bp, synthetic construct
  source 1..558: mol_type = other DNA
gcggtgagtgcggcggggggcggccgcggccagtgggggggggcgctggagttggggcggcagggccgagcggcccggggcggggagtcgctgtcctcgccgagcgcgcgggcgcacgggggcgcaggtgagccgcgggcggggcgctgcggctgggggctgggggcggcagggcggcttcgtgtgccactcggcctcggcaggccagctcttcgagctccgtgtccctggctctgtcctccttgggaccccacaagtgccctcaggaaggctgtggggttcccctgcgccgaggcccacccgtggccatgcgctaggaggtgtctcccacccgccggagtcccaaggacccctcccaagagctcgggcaccctgcggccatcacacccaacaggcgagtcggggtgtaggaagtccactgctcacaagggcaccccctcattaaacatcagaaattggagacaccccggatggatgggggccttggccccaaaccctttttctgtcccacctgtttccgtgcccctacacctcctgtgggtcttttcttgtctgtga

SEQ ID NO: 283 - protein, 185 aa, synthetic construct
AVSAAGGGRGQWGGALELGRQGRAARGGESLSSPSARAHGGAGEPRAGRCGWGLGAAGRLRVPLGLGRPALRAPCPWLCPPWDPTSALRKAVGFPCAEAHPWPCARRCLPPAGVPRTPPKSSGTLRPSHPTGESGCRKSTAHKGTPSLNIRNWRHPGWMGALAPNPFSVPPVSVPLHLLWVFSCL

SEQ ID NO: 284 - DNA, 228 bp, synthetic construct
  source 1..228: mol_type = other DNA
cctcagcctgggcgcctccagttcgggctttctgcctactcagcaacttctaatttgggtgcgtggttgggagatgctctcagctgtcagtcctgcccttggggggccagcttcctgcctctcacagccattaagtgcagctggacgcaggacccctgtcccactcctgggctgcaggagccacaggtgagcggtcggccgttgttcggctgctaccctgatgcctga

SEQ ID NO: 285 - protein, 75 aa, synthetic construct
PQPGRLQFGLSAYSATSNLGAWLGDALSCQSCPWGASFLPLTAIKCSWTQDPCPTPGLQEPQVSGRPLFGCYPDA

SEQ ID NO: 286 - DNA, 333 bp, synthetic construct
  source 1..333: mol_type = other DNA
gcggggctgactccccgcccagccctaatcctgacacaagcttttctgcctgttgcaccgagggggaccctcgtcctcggacctgagggcacaagaggtgcagggaggggctcgtggtgcacatactgcgtcccaggaggggtgggggtccctaagcagtgtcctcgcgcaggactcctaccggaagcaagtggtcatcgatggggagacgtgtctgctggacatcctggacacggcgggccaggaggagtacagcgccatgcgggaccagtacatgcgcaccggcgagggtttcctctgcgtgtttgccatcaacaacaccaagtcctttga

SEQ ID NO: 287 - protein, 110 aa, synthetic construct
AGLTPRPALILTQAFLPVAPRGTLVLGPEGTRGAGRGSWCTYCVPGGVGVPKQCPRAGLLPEASGHRWGDVSAGHPGHGGPGGVQRHAGPVHAHRRGFPLRVCHQQHQVL

SEQ ID NO: 288 - DNA, 288 bp, synthetic construct
  source 1..288: mol_type = other DNA
agacatccaccagtaccggtgagctgccagcacccgcgcaggccgtcccttctggcgccctggacgcagcctgccggtggctcacaccatcctccttgcagggagcagatcaagcgggtgaaggactcggacgacgtgcccatggtgctggtgggaaacaagtgtgacctggctgcacgcactgtggagtctcggcaggcacaggacctggcccgcagctacggcatcccctacatcgagacctcggccaagacgcgccaggtgagctggctccctgcccacctgtag

SEQ ID NO: 289 - protein, 95 aa, synthetic construct
RHPPVPVSCQHPRRPSLLAPWTQPAGGSHHPPCREQIKRVKDSDDVPMVLVGNKCDLAARTVESRQAQDLARSYGIPYIETSAKTRQVSWLPAHL

SEQ ID NO: 290 - DNA, 375 bp, synthetic construct
  source 1..375: mol_type = other DNA
gtcaatttacaaaaaataaaaaagggggagttgtatcccctgacgccccataattacctgtctcattctctctttattcaaaattttttgaccttggatgcccatggtaagagtgctgcagagcgcttttggcatccttctactgcccctcaggctttggtcaaatggaaagacccatttacaggctcttggcaaggcccagatctagtcctcatatggggccgagggcatgtttgtgtttttccacaggatgcagaaggccctcggtggctgccagaacgattggtgcgacatgtggaccctctacctgctgatgacattgattactctcagcaatgtagaagaagaccagacatattgggcctatgttcctga

SEQ ID NO: 291 - protein, 124 aa, synthetic construct
VNLQKIKKGELYPLTPHNYLSHSLFIQNFLTLDAHGKSAAERFWHPSTAPQALVKWKDPFTGSWQGPDLVLIWGRGHVCVFPQDAEGPRWLPERLVRHVDPLPADDIDYSQQCRRRPDILGLCS

SEQ ID NO: 292 - DNA, 234 bp, synthetic construct
  source 1..234: mol_type = other DNA
ggcgtgagtgtcattgacatagtctggaatctcaggaccttcccatacagcagggtggagaataggtggatcaggtacgtaggcccaatacgtctggtcttcttctgcattgctgagggtcatcaatgtcatcagcaggtagagggtccacatgtcgcaccaatcgttctggcagccaccgagggccttctgtatcctgtggaaaaacacaaacatgccctcggccccatatga

SEQ ID NO: 293 - protein, 77 aa, synthetic construct
GVSVIDIVWNLRTFPYSRVENRWIRYVGPIRLVFFCIAEGHQCHQQVEGPHVAPIVLAATEGLLYPVEKHKHALGPI

SEQ ID NO: 294 - DNA, 321 bp, synthetic construct
  source 1..321: mol_type = other DNA
ccggagccattatctgttttaagttttttaggagtggcagaagggtgtggtaacccscadmtggtcaaatggaaagacccacttacgggctcttggcaaggcccagatccagtcctcatatggggccgagggcatgtttgtgtttttccacaggatacagaaggccctcggtggctgccagaacgattggtgcgacatgtggaccctctacttgctgatgacattgatgaccctcagcaatacagaagaagaccagacgtattscadmcaagcagatacattaacagattttttagaccagtctctagtcccatcttgtaa

SEQ ID NO: 295 - protein, 106 aa, synthetic construct
PEPLSVLSFLGVAEGCGNPXXVKWKDPLTGSWQGPDPVLIWGRGHVCVFPQDTEGPRWLPERLVRHVDPLLADDIDDPQQYRRRPDVXXXSRYINRFFRPVSSPIL

SEQ ID NO: 296 - DNA, 246 bp, synthetic construct
  source 1..246: mol_type = other DNA
agggtccacatgtcgcaccaatcattctggcagccaccgagggccttctgcatcctgtggaaaaacacaaacatgccctcggccccatatgaggactggatctgggccttgccaagagcctgtaagtgggtctttccatttgaccaaagcctgagtggcagcagaaggatgccaaaagcgctccgcagcactcttaccatgggcatccascadmctctagtcccgtcttgtaaatcagtcacctga

SEQ ID NO: 297 - protein, 81 aa, synthetic construct
RVHMSHQSFWQPPRAFCILWKNTNMPSAPYEDWIWALPRACKWVFPFDQSLSGSRRMPKALRSTLTMGIXXXLVPSCKSVT

SEQ ID NO: 298 - DNA, 306 bp, synthetic construct
  source 1..306: mol_type = other DNA
gggtcatcaatgtcatcagcaggtagagggtccacatgtcacaccaatcgttctggcagccaccgagggccttctgtatcctgtggaaaaacacaaacatgccctcggccccatatgaggactggatatgggcscadmatttgtggccagcttaattcaagaaagccgtttggaagctcgaaaatattatgggaaagagccagatttgattgttgttccttttacaaaaacacagattcaaggcttgatgcagtttacagacagttttcccatcgccttggctcattttgcaggaactttagataa

SEQ ID NO: 299 - protein, 101 aa, synthetic construct
GSSMSSAGRGSTCHTNRSGSHRGPSVSCGKTQTCPRPHMRTGYGXXICGQLNSRKPFGSSKILWERARFDCCSFYKNTDSRLDAVYRQFSHRLGSFCRNFR

SEQ ID NO: 300 - DNA, 993 bp, synthetic construct
  source 1..993: mol_type = other DNA
attgcttcagtttttcaacatcatgatccaatttttccttcaattgtgtcacatgctcctcttcctgcggtaccaaatgtctttactgatggatctaacaatggtgtcgctgtttatgcactcaataaacaaattaaaaagatccagacacctccagcttcagctcaaatagttgagcttcgagcagttcatatggtgttgcttgattttgcttcccagtcttttaatttattctctgacagccattatgtggttcgtgcagtcaaaaatttagaaacagtaccgtttattaataccagtaatcctgttattcaggatttatttcttcagatacaacaagccattcagctgcgctgtaaaaaattttatattggccatattagagctcactctagtcttccaggccctttagcagcaggcaatcaaattgcagattctgccacgcagcttattgccttaactcaaatagaaaaagcacaaaaggctcatagcctccaccatcaaaacagccagagcctaagattacagtataagatccccagagaagcagcacgccagattgtaaagcaatgtcctgactgttcacatttacagcctgtgcctcattatggagttaaccctcggggcttgcgtcccaatgatctgtggcagacggatgtgactcatatacctgaatttgggaaattaaaatacgtccatgtctctatagacacgttctctggctttgtaattacttctggtcaatcaggagaagctacgtctcatgttatcagacactgtcttgctgcttttgccatgattggcactcctaaaaaacttaaaacagataatggctccggctacaccagcaagaaatttgctttattttgccagcaattttcaattaatcatgttactggcattccttacaatccccaaggacaagggattgttgaacgcactcatggcacattaaaagtcattttacaaaaaataaaaaagggggagttatag

SEQ ID NO: 301 - protein, 330 aa, synthetic construct
IASVFQHHDPIFPSIVSHAPLPAVPNVFTDGSNNGVAVYALNKQIKKIQTPPASAQIVELRAVHMVLLDFASQSFNLFSDSHYVVRAVKNLETVPFINTSNPVIQDLFLQIQQAIQLRCKKFYIGHIRAHSSLPGPLAAGNQIADSATQLIALTQIEKAQKAHSLHHQNSQSLRLQYKIPREAARQIVKQCPDCSHLQPVPHYGVNPRGLRPNDLWQTDVTHIPEFGKLKYVHVSIDTFSGFVITSGQSGEATSHVIRHCLAAFAMIGTPKKLKTDNGSGYTSKKFALFCQQFSINHVTGIPYNPQGQGIVERTHGTLKVILQKIKKGEL

SEQ ID NO: 302 - DNA, 381 bp, synthetic construct
  source 1..381: mol_type = other DNA
cccctgacgccccataattacctgtctcattctctctttattcaacattttttgaccttggatgcccatggtaagagtgctgcagagcgcttttggcatccttctactgccactcaggctttggtcaaatggaaagactcacttacaggctcttggcaaggcccagatccagtcctcatatggggccgagggcatgtttgtgtttttccacaggatgcagaaggccctcggtggctgccagaacgattggtgcgacatgtggaccctctatttgctgatgascadmgccatgcactgtgtccgcgtcccgctcgctaccattgggaaccagcagcagccgctgcagctctcgcccctgaaggggctcagcctagcggataa

SEQ ID NO: 303 - protein, 126 aa, synthetic construct
PLTPHNYLSHSLFIQHFLTLDAHGKSAAERFWHPSTATQALVKWKDSLTGSWQGPDPVLIWGRGHVCVFPQDAEGPRWLPERLVRHVDPLFADXXXHALCPRPARYHWEPAAAAAALAPEGAQPSG

SEQ ID NO: 304 - DNA, 270 bp, synthetic construct
  source 1..270: mol_type = other DNA
tgcaaccaatgtccagtgacccagattgcgctgaactttgatgtgtttaccactaggtggagcggtttagccaagaagttcagattacagaagcccgctgtttctatggcttccaaattgccatggaaaacatacattctgagatgtatagtctcctcattgacacttacatcaaagattccaaggaaaggtgagtatttgagtggtatgccaacatgtttgggactcactaattgtttatttcaagtttttggattcagaccgggatag

SEQ ID NO: 305 - protein, 89 aa, synthetic construct
CNQCPVTQIALNFDVFTTRWSGLAKKFRLQKPAVSMASKLPWKTYILRCIVSSLTLTSKIPRKGEYLSGMPTCLGLTNCLFQVFGFRPG

SEQ ID NO: 306 - DNA, 285 bp, synthetic construct
  source 1..285: mol_type = other DNA
gcacgctcgggccgggttggggtggcgggtacctgggggactcgggcatgcctctcaccgcatgtctccccgcagccacccgctctcaacggcacccgcgtgctggccagcaaggcggcccggaggatcttccaggaggcggcggagtccgtggagccggtgagcggatgcccgagggcggagacagcgcagtgggcgtggccagcgcgcagcgcctgggggcgacagccgacttcgccggctctctggcgccatggctttctttgtctttctacttactcataa

SEQ ID NO: 307 - protein, 94 aa, synthetic construct
ARSGRVGVAGTWGTRACLSPHVSPQPPALNGTRVLASKAARRIFQEAAESVEPVSGCPRAETAQWAWPARSAWGRQPTSPALWRHGFLCLSTYS

SEQ ID NO: 308 - DNA, 291 bp, synthetic construct
  source 1..291: mol_type = other DNA
cagctgacacgtaagacactggaccacatgaaattgccgacaattgaatgtaactggatgggaaaaatggcaatttcatatggttcgatggatacttcacattttcattactttctcccccaacagaaaactaaggtgtctgccctcagcgggcaggatgaaccactgctgagagaaaacccccgccgctttgtcgtctttcccatcgaataccatgatatctggcagatgtataagaaagcggaggcttccttttggacagctgaggaggtaatcagattcaggagctag

SEQ ID NO: 309 - protein, 96 aa, synthetic construct
QLTRKTLDHMKLPTIECNWMGKMAISYGSMDTSHFHYFLPQQKTKVSALSGQDEPLLRENPRRFVVFPIEYHDIWQMYKKAEASFWTAEEVIRFRS

SEQ ID NO: 310 - DNA, 285 bp, synthetic construct; identical to SEQ ID NO: 306
  source 1..285: mol_type = other DNA

SEQ ID NO: 311 - protein, 94 aa, synthetic construct; identical to SEQ ID NO: 307

SEQ ID NO: 312 - DNA, 291 bp, synthetic construct; identical to SEQ ID NO: 308
  source 1..291: mol_type = other DNA

SEQ ID NO: 313 - protein, 96 aa, synthetic construct; identical to SEQ ID NO: 309

SEQ ID NO: 314 - DNA, 255 bp, synthetic construct
  source 1..255: mol_type = other DNA
ctttggatgcccatggtaaaagtgcagctgaacgtttttggcatccttcaactagccctcaggccttggtcaaatggaaggacccacttacgggtgtctggcaaggcccagatccagtcctcatatgggggcgagggcatgtttgtgtttttccacaggatgcagaaggccctcggtggctgccagaacgattggtgcgacatgtggaccctctacctgctgatgacattgatgascadmctccgcttcagctag

SEQ ID NO: 315 - protein, 84 aa, synthetic construct
LWMPMVKVQLNVFGILQLALRPWSNGRTHLRVSGKAQIQSSYGGEGMFVFFHRMQKALGGCQNDWCDMWTLYLLMTLMXXLRFS

SEQ ID NO: 316 - DNA, 270 bp, synthetic construct
  source 1..270: mol_type = other DNA
gacagggagctgatgaatcttttcaagattttgtgtctcgccttactgttgctgcgggacggacctttggagcgtccgtggctacggaggctttcattaaacagcttgcttatgaaaatgcaaattctgcctgccaagcgattattaggcccattaagaaaaaaggcactatctctgattttatccgttcctgtgccgatgtcggcccctccttttcacagggagtggccctggctgccgctttacaaggaaaaagcattcatgaagtaa

SEQ ID NO: 317 - protein, 89 aa, synthetic construct
DRELMNLFKILCLALLLLRDGPLERPWLRRLSLNSLLMKMQILPAKRLLGPLRKKALSLILSVPVPMSAPPFHREWPWLPLYKEKAFMK

SEQ ID NO: 318 - DNA, 240 bp, synthetic construct
  source 1..240: mol_type = other DNA
tgcagcaacaggccaagcttcatgctagtggccgcgcaggagcttgttttaactgtggaaaaatgggacatcgagcttctcaatgcccacataaaatggaggctaacaatccgtcggctactgctgtggttaaaaaacctccagggccttgtcccaggtacaagaaaggcgctcattgggctaataaatgtaaatccaaaactgacaaagacggcaaacccttacagggaaactgggtga

SEQ ID NO: 319 - protein, 79 aa, synthetic construct
CSNRPSFMLVAAQELVLTVEKWDIELLNAHIKWRLTIRRLLLWLKNLQGLVPGTRKALIGLINVNPKLTKTANPYRETG

SEQ ID NO: 320 - DNA, 315 bp, synthetic construct
  source 1..315: mol_type = other DNA
ttacttgtctttttattcaaaatttttttgactttggatgcctatgttaagagtgcagctgaacgtttctggcatccttctgccgtccctgaggctttggtcagaaagaaggatccacttactggatcatggcaaggcccagacccagtcctcatatggggccgagggcatgtttgtgtttttccacaggatgcagatagtcctcggtggttgccagaacgattggtgcgacatgtggaccctctacctgctgatgacattgatgaccctcagcaatacagaagaagaccagacgtattgggcctacgtacctga

SEQ ID NO: 321 - protein, 104 aa, synthetic construct
LLVFLFKIFLTLDAYVKSAAERFWHPSAVPEALVRKKDPLTGSWQGPDPVLIWGRGHVCVFPQDADSPRWLPERLVRHVDPLPADDIDDPQQYRRRPDVLGLRT

SEQ ID NO: 322 - DNA, 801 bp, synthetic construct
  source 1..801: mol_type = other DNA
tggacacatgaaacaacatttggaaagttttgtaaatcaggcactccctgcagtcaggtgactgatttacaagacgggactagagactggtctaagaaatctgttaatgtatctgcttgtgttccttccccttatacacttttgattggaaatattaatgtacattttgtaggagttcagtttatggaagatgtgattcagagtataaaagttaaatcttatttaaaatgtcattcagaatatcattggatatgtgttacttcscadmccccggcgacggggcgcgcggggggcggggcggactgtgcccagtgcgccccgggcgggtcgcgccgtcgggcccggggggtttccaggcgccacgccgtgaccaaagcacagcgaagcgagcgcacggggtcagcggcgatgtcggccacccacccgacccgtcttgaaacacggaccaaggagtctaacacgtgcgcgagtcaggggctcgcacgaaagccgccgtggcgcaatgaaggtgaaggccggcgccgctcgccggccgaggtgggatcccgaggcctctccagtccgccgagggcgcaccaccggcccgtctcgcccgcagcgccggggaggtggagcacgagcgcacgtgttaggacccgaaagatggtgaactatgcctgggcagggcgaagccagaggaaactctggtggaggtccgtagcggtcctgacgtgcaaatcggtcgtccgacctgggtataggggcgaaagactaatcgaaccatctagtagctggttccctccgaagtttccctcaggatag

SEQ ID NO: 323 - protein, 266 aa, synthetic construct
WTHETTFGKFCKSGTPCSQVTDLQDGTRDWSKKSVNVSACVPSPYTLLIGNINVHFVGVQFMEDVIQSIKVKSYLKCHSEYHWICVTSXXPATGRAGGGADCAQCAPGGSRRRARGVSRRHAVTKAQRSERTGSAAMSATHPTRLETRTKESNTCASQGLARKPPWRNEGEGRRRSPAEVGSRGLSSPPRAHHRPVSPAAPGRWSTSARVRTRKMVNYAWAGRSQRKLWWRSVAVLTCKSVVRPGYRGERLIEPSSSWFPPKFPSG

SEQ ID NO: 324 - DNA, 273 bp, synthetic construct
  source 1..273: mol_type = other DNA
ccttggatgcccatggtaagagtgctgcggagcgcttttggcatccttctgctgccactcaggctttggtcaaatggaaagacccacttacaggctcttggcaaggcccagatccagtcctcatatggggccgagggcatgtttgtgtttttccacaggatgcagaaggccctcggtggctgccagaacgattggtgcgacatgtggaccctctacctgctgatgacattgatgaccscadmgttgagggtcatcaatgtcatcagcaagtag

SEQ ID NO: 325 - protein, 90 aa, synthetic construct
PWMPMVRVLRSAFGILLLPLRLWSNGKTHLQALGKAQIQSSYGAEGMFVFFHRMQKALGGCQNDWCDMWTLYLLMTLMTXXLRVINVISK

SEQ ID NO: 326 - DNA, 450 bp, synthetic construct
  source 1..450: mol_type = other DNA
agggtccacatgtcgcaccaatcgttctggcagccaccgagggccttctgcatcctgtggaaaaacacaaacatgccctcggccccatatgaggactggatctgggccttgccaagagcctgtaagtgggtctttccatttgaccaaagcctgagtggcagcagaaggatgccaaaagcgctccgcagcactcttaccatgggcatccaaggtcaaaaaattttgaataaagagagaatgscadmgaccgggccgggctcatcgcccggcggccgccgccgccgctttctcgttaatgatccttccgcaggttcacctacggaaaccttgttacgacttttacttcctctagatagtcaagttcgaccgtcttctcagcgctccgccagggccgtgggccgaccccggcggggccgatccgagggcctcactaaaccatccaatcggtag

SEQ ID NO: 327 - protein, 149 aa, synthetic construct
RVHMSHQSFWQPPRAFCILWKNTNMPSAPYEDWIWALPRACKWVFPFDQSLSGSRRMPKALRSTLTMGIQGQKILNKERMXXTGPGSSPGGRRRRFLVNDPSAGSPTETLLRLLLPLDSQVRPSSQRSARAVGRPRRGRSEGLTKPSNR

SEQ ID NO: 328 - DNA, 321 bp, synthetic construct
  source 1..321: mol_type = other DNA
tcgtcttcgaacctccgactttcgttcttgattaatgaaaacattcttggcaaatgctttcgctctggtccgtcttgcgccggtccaagaatttcacctctagcggcgcaatacgaatgcccccggccgtccctcttaatcatggcctcagttccgaaaaccaacaaaatagaaccgcggtcctattccatscadmttgctgagggtcatcaatgtcatcagcaggtagagggtccacatgtcgcaccaatcgttctggcagccaccgagggccttctgcatcctgtggaaaaacacaaacatgccctcggccccatatga

SEQ ID NO: 329 - protein, 106 aa, synthetic construct
SSSNLRLSFLINENILGKCFRSGPSCAGPRISPLAAQYECPRPSLLIMASVPKTNKIEPRSYSXXXAEGHQCHQQVEGPHVAPIVLAATEGLLHPVEKHKHALGPI

SEQ ID NO: 330 - DNA, 279 bp, synthetic construct
  source 1..279: mol_type = other DNA
ccaaagcctgagtggcagtggaaggatgccaaaagcgctccgcagcactcttaccascadmtgtcatcagcaggtagagggtccacatgtcgcaccaatcgttctggcagccaccgagggccttctgcatcctgtggaaaaacacaaacatgccctcggccccatatgaggactgggtctgggccttgccatgatccagtaagtggatccttctttctgaccaaagcctcagggacggcagaaggatgccagaaacgttcagctgcactcttaacatag

SEQ ID NO: 331 - protein, 92 aa, synthetic construct
PKPEWQWKDAKSAPQHSYXXXSSAGRGSTCRTNRSGSHRGPSASCGKTQTCPRPHMRTGSGPCHDPVSGSFFLTKASGTAEGCQKRSAALLT

SEQ ID NO: 332 - DNA, 555 bp, synthetic construct
  source 1..555: mol_type = other DNA
cactgcccttccttcgagcacaggctgacctcagtgacagatgaactggctgcggtcaccgcagtggtgttcagccggcaggaggtggtcacccagctgcagcgcgagctgcggaatgaggaacagaacatccacccccggcagcggtcagtgggtcccacctattgtagccttgtgcccgcgccccaccccacacacctgccctgcagccagctgcaggctgagccctctctctgccccctcccacctcccacctgcctgtctcctttcagggtttacctgctgggcaagaggcaggtattgcaggaggagctccaggggctgcaggtggcactgtgcagccaggccaagctggaggcccagcaggatcttttgcaggccaagctggagcagctgggccccggggatcccccgcctgtgccgctcctacaggacgaccgccactctacctcctcctcggtgagtgccctactgccctccgtggtcaccttgctgccagcccaggctgtgtcctcattttcgccctccccctccccaagcctggccacccgctga

SEQ ID NO: 333 - protein, 184 aa, synthetic construct
HCPSFEHRLTSVTDELAAVTAVVFSRQEVVTQLQRELRNEEQNIHPRQRSVGPTYCSLVPAPHPTHLPCSQLQAEPSLCPLPPPTCLSPFRVYLLGKRQVLQEELQGLQVALCSQAKLEAQQDLLQAKLEQLGPGDPPPVPLLQDDRHSTSSSVSALLPSVVTLLPAQAVSSFSPSPSPSLATR

SEQ ID NO: 334 - DNA, 231 bp, synthetic construct
  source 1..231: mol_type = other DNA
cgtctgtccctggcctcaggagcaggagcgggaaggggtacggacgcctaccctggagctcctgaagagccacatctcaggaatctttcgccccaagttttcggtgagtggcacctgtctgggcctgcgcctctgcccttctccaaggggtgggctgggccaggggtctcagacatgcccccactgcaccccgcccacatggtgttctggttagcccctgggttgccctaa

SEQ ID NO: 335 - protein, 76 aa, synthetic construct
RLSLASGAGAGRGTDAYPGAPEEPHLRNLSPQVFGEWHLSGPAPLPFSKGWAGPGVSDMPPLHPAHMVFWLAPGLP

SEQ ID NO: 336 - DNA, 258 bp, synthetic construct
  source 1..258: mol_type = other DNA
agaggaggctctctccacgccgcttttattggggtgccaagcaccaacgtccccagatcctgccactctcacacccccttcttctctgccatcacatgtgctgaagggactcacagctttagtgaccccatggctctccctgctccaggagtggttggggggccgcagcctggtggaaaaggcaaaagtttggtttgggaccagtcagccggcccccccatcccagctgtgcctgggccagtctatggcctgctctag

SEQ ID NO: 337 - protein, 85 aa, synthetic construct
RGGSLHAAFIGVPSTNVPRSCHSHTPFFSAITCAEGTHSFSDPMALPAPGVVGGPQPGGKGKSLVWDQSAGPPIPAVPGPVYGLL

SEQ ID NO: 338 - DNA, 297 bp, synthetic construct
  source 1..297: mol_type = other DNA
cgtgagtggtgccaggacccgcgcccaccctgccccacccttccctgtcaccagaatgaccttgagagggtaggaagaaaggggctgctagtcttagatgctagtcagagctgcaaggggccatggagaccacttagtccctataacagaacaggcgtaagtagcatgggtagcaggtgtgttgggcgccatgaggtcgtgccttcctgcagtgtctctgcctctcgtcccaggcaggccctttctccctgctactctcccgctcccctcccagggctcaggccccctcagcagtag

SEQ ID NO: 339 - protein, 98 aa, synthetic construct
REWCQDPRPPCPTLPCHQNDLERVGRKGLLVLDASQSCKGPWRPLSPYNRTGVSSMGSRCVGRHEVVPSCSVSASRPRQALSPCYSPAPLPGLRPPQQ

SEQ ID NO: 340 - DNA, 315 bp, synthetic construct
  source 1..315: mol_type = other DNA
aggctgctgaccccaagttgccctgccctgcagaacctgtaccgactggaaggtgatggttttcccagcgtccccttgctcattgaccacctgctgcagtcccagcagcccctcaccaagaagagcggtattgtcctgaacagagctgtgcccaaggtgagcctgcaccccaccggcccacaccacccaccacagggtttggggagcgcgggttcaggcccacagaatcggggcaggaggggctttccaggtctctggtctacggtctgggtaccacgcgactcctcactctccaaggggtcagctccctcctag

SEQ ID NO: 341 - protein, 104 aa, synthetic construct
RLLTPSCPALQNLYRLEGDGFPSVPLLIDHLLQSQQPLTKKSGIVLNRAVPKVSLHPTGPHHPPQGLGSAGSGPQNRGRRGFPGLWSTVWVPRDSSLSKGSAPS

SEQ ID NO: 342 - DNA, 270 bp, synthetic construct
  source 1..270: mol_type = other DNA
gtgcgggcacggcctcgtgctgcccacgccagccccccagtaaccccgcccaagcacaggccatgctgtcaccccgtgccccctttcccgagggaccatgagtcctgggcagggagcggcccttgttcatgtctatgtgtggagtccccagctcagggaggtgacgggtgcggtgtgtggtggctgagtgagcccctttcctgctttatccagggaccttgctgctcggaactgcctggtcacagagaagaatgtcttgaagatcagtga

SEQ ID NO: 343 - protein, 89 aa, synthetic construct
VRARPRAAHASPPVTPPKHRPCCHPVPPFPRDHESWAGSGPCSCLCVESPAQGGDGCGVWWLSEPLSCFIQGPCCSELPGHREECLEDQ

SEQ ID NO: 344 - DNA, 246 bp, synthetic construct
  source 1..246: mol_type = other DNA
ggctgggcgtgcctctggctgatggacgtgggtggctcactcacactgcctcacctccttgcaggccgctattcgtccgagagcgatgtgtggagctttggcatcttgctctgggaggccttcagcctgggggcctccccctaccccaacctcagcaatcagcagactcgggagttcgtagaaaaaggtaaggcaaccccactgcatgacagcagcccgacccacgcgctcatcccagtgctatag

SEQ ID NO: 345 - protein, 81 aa, synthetic construct
GWACLWLMDVGGSLTLPHLLAGRYSSESDVWSFGILLWEAFSLGASPYPNLSNQQTREFVEKGKATPLHDSSPTHALIPVL

SEQ ID NO: 346 - DNA, 438 bp, synthetic construct
  source 1..438: mol_type = other DNA
cgctgtgttcaggctcatggagcagtgctgggcctacgagcccagtcagcgacccagcttcagcaccatctaccaggagctgcagaccatccgaaagcggcatcggtgaggctcggcccgcttctcaagccagtggcttctgttggcaagattatacctcctccccagctccagctcacaccgtgggacagcccttcccagtcctggactctggccgccggcatccatgctgccaggggggatgcagctccatgtctgctgtgcgtccccattcctgccagscadmgatttaacctttatgctttgaatgacatctcccatatactgaactcctacaaaatgtacattaatatttccaatcaaaagtgtatatggggaaggaacacaagcagatatattaacagatttcttagaccagtctctagtcccgtctggtaa

SEQ ID NO: 347 - protein, 145 aa, synthetic construct
RCVQAHGAVLGLRAQSATQLQHHLPGAADHPKAASVRLGPLLKPVASVGKIIPPPQLQLTPWDSPSQSWTLAAGIHAARGDAAPCLLCVPIPAXXRFNLYALNDISHILNSYKMYINISNQKCIWGRNTSRYINRFLRPVSSPVW

SEQ ID NO: 348 - DNA, 228 bp, synthetic construct
  source 1..228: mol_type = other DNA
gggtcatcaatgtcatcagcaggtagagggtccacatgttgcaccaatcgttctggcagccaccgaggactatctgcatcctgtggaaaaacacaaacatgccctcggccccatatgaggactgggtctgggccttgccatgatccagtaagtggatccttccttctgaccaaagcctcagggacggcagaaggatgccagaaacgttcagctgcactcttaacatag

SEQ ID NO: 349 - protein, 75 aa, synthetic construct
GSSMSSAGRGSTCCTNRSGSHRGLSASCGKTQTCPRPHMRTGSGPCHDPVSGSFLLTKASGTAEGCQKRSAALLT

SEQ ID NO: 350 - DNA, 20 bp, synthetic construct
  source 1..20: mol_type = other DNA
tggtgaaggtcggagtgaac

SEQ ID NO: 351 - DNA, 20 bp, synthetic construct
  source 1..20: mol_type = other DNA
gaaggggtcattgatggcga

SEQ ID NO: 352 - DNA, 6523 bp, Scotophilus bat coronavirus
  source 1..6523: mol_type = other DNA
caattgcttggttgcattgcctaagttgcaaggtcttactaccactctgtcttttgactcaccacttaatgtgcctgggttttcctgtaacggcgccaatggttctagctcagcggaagcctttcgttttaacgtcaatgatactaagttgtttgttggtgctggcgctgttacattgaacaccgtcgatggtgttaatgtttctattgtgtgctccaataatgcaacacagcccactaggtcaaacaacttgcaggaagacctgccttactattgcttcactaacactagtagcggcactaatcacactgttaagtttctttcagttttcccgccaatcattcgtgagtttgtgatcaccaaatatggcaatgtctatgttaatggctatatctatttgagaactagaccattgacagccgtgcacttgaacgcatcctctcattcgcaggacgtagcagggttttggactattgccgccacaaacttcacggatgtgcttgttgaggtgaacaacacaggcattcagaggttgttgtattgtgacacgcctgaaaacagtgtcaaatgttcacaactctcttttgaactggaggacgggttttattccatgactgcagataatgtttatgcagtaactaagccccacacgtttgtgactttgcccacgtttaatgaccatgggttcgttaatgttactgtgggtggtaactttgacagttcatacccaccaaagttcactgctaatggcaccttagttaataacggcactgtggtgtgtgtcacttctaatcagttcacccttagacacgactttatggtaggttattctgctgatatgcgtaagggtatatttgagtactctagtacatgccctttcaatagagaaactatcaataactaccttacgtttggtcgtatttgtttctctacttcaccggcggacggtgcttgcgaattgaagtactatgtttggaacaccattggagccgtttcacacctggctggcaccttgtatgttcaacatacaaagggtgacataataactggtacacccaaaccattgcagggtttgaatgacatttctgaattgcacctagacacgtgcaccacttacaccatttatggttttaggggtgacggtgttattaggttgaccaatcaaactttcttgtcaggtgtctactacacttcagagagtggtcagttattagcttttaagaatgtcactacagggcagatttattctgttacaccctgccaactggttcagcaggttgcttttgttgaggataggattgttggcgtcattagtagtgctaataatactgggttctttaattccacaagaacatttccaggcttctattatcactctaatgacaccaccaattgcacctcaccaagacttgtttactctaatataggtgtttgtactagtggtgccataggtttgctgtctcctaaagctgcacaacctcaggttcaacccatgttccagggtaatattagtatccctactaattttactatgagtgtgcgcactgagtatatacagttgtttaacaaacccgtttctgtagactgcgcaatgtatgtctgcaatggtaatgaccgttgtaagcaattgttgtctcagtacacttcagcatgcaagaacatagaatctgcgctgcagctcagcgcaaggttggaatcaatggaggttaactctatgttgacagtttcagatgaggcacttaagcttgccactataagccaatttcctggtggtggttataattttaccaatattcttccagcaaatcctggtgctaggtcagttattgaagacattttgttcgataaagttgtcactagtggtttgggcacagttgatgaagattataaacgctgcagtaatggactgtctattgcagatttagcttgtgcgcagcactataacggcattatggtgttgccgggtgttgcggactgggaaaaggtccatatgtactcggcttcacttgtcggtggtatgaccttaggtggtatcacttctgctgcggctttgcctttctcatatgcagtgcaggcaagacttaattatgttgcactacagaccgacgtgctgcaacgtaatcaacaaatgctagccaattcctttaatagtgctattagtaacatcacattagcttttgagagtgtcaataacgctatctatcaaacttctgctggtttgaatacggtagcagaggcactttcaaaagtacaggatgttgtgaatggtcaaggaaatgcactcagtcaactaacagtccaattgcagaataattttcaagctatttccaattctattggtgacatttatagtaggttagatcagataactgctgatgcgcaagttgacagacttatcacaggtcggcttgcagctcttaatgcctttgttgcacagtcacttaccaagtatgcagaagtgcaagctagtaggacattggccaagcaaaaggttaacgagtgtgttaagtcacagtcccccagatacggtttctgtggtgatgaaggggaacatattttctcactcacccaagctgctccacagggtctgatgttcctacacaccgttttagtacctaatggttttattaacgttacagcagttacaggtttatgtgttgatgagaccatagctatgacattacgtcagagtggatttgtcttgtttgtgcaaaatggtaattatctcgtgtcaccgaggaaaatgtttgaacctcggagacctgaagttgctgattttgtgcaagtaaaaacatgcacgattagttatgttaacatcaccaataaccagttgcctgacattattccagattatgtagacgttaataagactatagatgagattttggccaacctacctaataatactgtgcctgatttgccacttgatgtctttaatcaaacatttcttaatctcactggtgagattgcagaccttgaagcgcgatctgaatcccttaaaaacacatcagaagaacttagacagttgatccaaaatattaacaacacacttgtagaccttcagtggcttaatagggttgagacctttattaagtggccgtggtacgtgtggttggctattgttatagctcttattttggttgtttcactgcttgtgttctgctgtatatctacaggttgttgcggttgttgcggttgttgtggttcttgtttctcaggttgttgtcgtggaactaaacttcaacattacgaaccaatagaaaaggttcatgtgcaataatgtttcttggtctgttccagtatactattgatactgcagttgagcacactgtagaacatgctaacttgtcccaagaagaggctttgatgttggaagaaaacatcgttcctctgagacaagctacacatgttactggatttttgctcaccagtgtttttgtttacttctttgcactgtttaaggcttcaagctacaaacgtaatttgctgctatttttagcacgtttgttagctttattaatttatgcacccattttaatattttgtggtgcatacttggacgcttttatagtagtcgcaacattgacttctcgtctattgtttttgacctactactcatggcgttataaaacttataaatttcttatttacaactcttccacacttatgtttttacatggtcatgccaattattataatggcaggccctatgtaatgcttgaaggtggaagccattacgtcacattgggtactgatatagtaccattcgtcagccgaagtaatctctatcttgccattcgtggtagtgctgagtcagatatccaactgttgagaactgtcgagttgttagatggtaattacctctacattttctccagttgtcaagtcgttggtgttactaattcaggttttgaggagattcaactagacgaatatgctacaattagtgaatgataatggtgtagttgtaaatgcgattctctggctttttgtactcttttttgtgctagttattagcattactttcgtccaacttataaacctttgttttacttgccaccggttgtgtaataacgttgtttataagcctgttggaaaagtatacggagtatacaagtcttatatgcgaattcaacccttgacatctgacattattcaagtataaacgaaaatgtcttcgaaccaatccgttcctgtagaggaggtgattaaacacctcagaaattggaacttttcatggaatatcatacttacaatactcttagtagtgttgcagtatggacattacaaatattccagggttctctatggcttaaagatggccattctttggcttctttggccacttgttctggccctttccatctttgatgcctgggccagttttaatgttaattgggttttcttcgcattcagcatcctaatggcctgcgtcacagctgtgctgtggattatgtactttgttaacagtatcaggttgtggcgacgcacccattcttggtggtcctacaatcctgaaacggactctattctgtctgtctctgtgctgggtcggcatgtctgcctaccaatacttggtgcacccacgggcgtaacgctcacactgcttaatggcacattgcttgtagaaggctatcaggttgctactggcgtacaggtaaataatttacctggttacgtaacagtcgccaaagcttcaacaacaattgtctaccagcgtgtgggacgttccatgaatgcaaattcaagtactggctgggctttcttcgtgaagtccaagcatggcgactactatgctgctgcgaatccaacagaggttgtaacagatagtgagaaaattctacatttagtctaaacagaaacttatggcttctgtaaaattccaacctcgtggtcgttccaagggacgtgttcctctgtctctttttgctccacttagggttactgatgaaaaaccactttacaaggtcctaccaaataatgccgtccctcagggaatgggaggtaaggaccaacaaattggatactgggttgaacaacagcgctggagaatgcgccgcggagacagagttgacctgccatctaactggcacttctacttcctcggtactggaccgcattctgatttgcctttcagaaaacgcactgatggtgttttctgggttgcaatcgatggtgctaagacccagccaacaggccttggcgtacgtaagtcgtctgagaagccgttggttccaaaatttaagaacaaattacccaataatgtggaaatcgttgaacctaccacaccaaacaactccagagctaactcaaggagtcgtagtcgtggtggacagtccaacagcagaggaaattcccaaaacagaggtgataaatccagaaaccagtccagaaacaggagtcaatctaatgatcgtgggtctgactcgcgagatgacttagtggctgccgttaaaaaagcacttgaagacctaggagttggtgctgcaaagccaaaaggcaaaacccagagtggtaaaaacacccctaagaacaaatctaggtcaggctctgtgcaacgtgcagaagccaaggacaaacccgagtggcgtcgtactcctagtggcgatgagtcagttgaggtttgttttggaccccgtggtggcaccagaaattttggtagctccgaatttgttgctaaaggtgtgaatgcccccggttatgctcaggctgcttcactggtacccggcgccgcagcactgctttttggtggtaatgttgccaccaaggaaatggctgatggtgttgaaatcacctatacatataaaatgttagtccctaaggacgacaagaaccttgaaatctttcttgctcaggttgacgcatacaagctcggcgatcccaagcctcagcgtaaagtcaaacgttcaagaaccccaacaccaaaacctgcaacagagccagtttatgacgacgttgctgcagatcctacttacgccaatcttgagtgggacaccacagtggaggatggtgttgagatgatcaacgaggtttttgacacccagaattgaattcaactaaaacaatgtacagaattgtagctattgttttggctgagctttttcgagcactggccatttttggctcattcttccaaatttttttgctatattttgattgcatttccaaggtgagtttaagctgtcctacaggacgttggtgtttgcttacatgtgctgatttccttattcttgtgctcatattctttcttttcttggtgcctttttcttactgtttagtggtgtacatcgttaaagatgattgggccccctggatgtggtatgttaacctctacaggcccctacatgatgccttaatcagatttcttatgacaccagactttgctgtcttggttttatctttcttgttcatgatcttaacatggctgctgggcattggaatcttccaatactagcggtcttggtcttgcacacaacggtaagcctgtaataatgacagtgcaagcaggttattattatattgc

SEQ ID NO: 353 - DNA, 6402 bp, synthetic construct
  source 1..6402: mol_type = other DNA
ctactgcagtatttctcagctagagttgtgctggcgactcacagtcacttgaggaactttacaaacctttacaggcctggactcctccctgaaggtttttgactgagtcaatctaataaggcctggacattgtgtatttagaaatagttccccgagtttctgatacaacccttgtttcaaaagtactgaatgtataagtgtaatgtatcacttcacagatttcaaaagcgtaagaaacctctagaaaaggtacagttagtgaggggtacttacttcatctctctcctttgcaacacgcttaggctgacttgaactgtctttaccagtgggctcgagaagtgaaagggatatcattggcggtatctgctggcctacagaagtacacctgtttcctttttgccatcagtcattcactggcgctcagagactctttagacatttgctgatgggtagtctactaataagtagaatccgacccttgaagaaagagttttgcatctctgttcacgctcaggtcgttagatcaataaatgtttaccaccagcatgcttttcctgcagcagtaagaaatcatgaaccttccttttagttgaagctgtgcgatagactgtctcaatatgtctgtttaattcctacagccctggcataatcaggagggtaggtttaaacatcaaaacatcaagaacctggagttcgtaccaaaacagaacggaactgtttcaataatttagaatcagcggtcctatgcaggaattgagtatttgattaacaatctgtgaaaaataaatgcaaatggactgtggtgtgaataagttttgaaaaattcctgaagtgggtaaaatgagtctaaatgacaagctgtctttattgcaagctgcggcccaatttttggataaaacgtaggataccaaagaaaggaaagattttacatacaaatattcacattatacacaaccatttggaaacagcaaatataaaatcccaagtgccctatagactaaatgtgtctcctggatatgtttaatttgcctccatgtattgatcaacgctgacaattttggagactcacgtagaatgtggatgtcaagcttctctaaagaatcaaacctggtccttcttgatcacttactgtgctgtgccttttagatcagacatgtgttctctagtttgctactgtccccacctgggttcttcacttactttggtcaccttctttgtctccataagcactggaggttacgattcttgtcttatagtatacgaggtctgacaattacattcgtgaattcattctagaaaaagtaatgcatatctcattggtgaatatcactgtggtcaccttcaaagtactccccttgggaagctgtgcactgatgccagcgcctagtccacccttcaaagcaattttggaactcttttcctggaatggtcatcagagctctcgtcgtgttacccttgatgtcctgaatgtcatcaaaatgttttcctttcaatatttcctttatcatcaggtaaagagagaaggcattgggggccaggtgaagtgagtagggagggtgttccaatacggttatttgtttgctggttaaaaactccctcacagactgtgtgagccggtgtattgtcatgatgcaaaatccatgaattgttggagaaacgttcaggccattttcgtctgaagtttttcacgcagccttttctgcacttctaaatagtaaacttggttaactgtttgtccagttggtacaaattcataatgaataatccctctgatatcaaaaaaggtcagcaacatcgtttggacccttgatttggactgatggaacttttttcttcgtggagaattggctgacttccattttgtactttgacattctgttataggatcatattggtacacccatgtttcatcaccagtgacaacatggcctaaaatgtcatgttgcctctccaaaaggtcttgacaaacttggactctcttttgtttttgttcaccggtgagctacttcgggaccatttttgcacacatcttcctcatgccaagattttcagttcagattttgtctttctctattgattttaccttggactactatgcttctcagagtcagccaataactttgatggatcatttgatgaatttttgcaatttttttcatcagttctactcgttattggatgccctgacctctctttatcagttgcacgttctctcccctcggaaaaaacgtttactccactagtacactgccattttattcttggcattatcctcatagacttggactaacacgtccctgatttcacttccactcttgccaggtttaccaagaaatttaatgtttgttcattgttctaattcaagctcagacattcttgcgatgcaacacaaaaacacacaacaacaataatgaatgccattcagcaacaccgccacatgtccacacggacacagctgtgagatttatataccaaggttatgaaaccttatcgagctgtttgtacagtgctgccaatgtaaacgcaaggtggcaagttctcgaacttaattttaagaccccatattttgacaaacatttcaagattttcaatacagtcaatgttctatgttgacttattatgagtattttatttaaatttttttattgtgctatataggggaacagtgtgtttctccagggcccatcagctccaagtcattgcccttcaatctaggtgtggagggcacagctcagctccaagtccagtcgccgtttttcaatctttagttgcagggggcgcagcccaccatcccatgcgggaattgaaccagcaaccttgttgttgagagctcacagtctaaccaactgagccattaggccaccccaacaaaacgtattgtttatttcagaagtgatacagaaaattaggtgaaaagagaaaaaatcattcatattcccaatatccaaagacaaaaacacagcactgcttcacattttaataaatttccttaaagtgtcttctctttatttaatctctacactacacttttgaaaactgacaaatttaggtttagtctctggcaatgatttctccctgtcttttagaagttttcttgtactgtgcctgactattaaacattggtattcttcaacttctgaccttaaaccccttctagtctcactcaatatgatcaatatgcccggctttctcccatctatgagctgataaatcccaaataaacatcttctatatacactactttctgatcattaaatccatttttccatttccttacagcataatccacgtggatgtctttaaatttaaacatccatgcctgccctttcttctgtactctcttcagattagtttgattgcaagtaagagaaagtcaaaatcaaattgtgtagaaaaaacaaaaacaaaaaactcaaaataaccttttggttccaggataagactcgttggatgcagaagctccaagtctcactgatcatgcgccatttctgttttgctagttccttctcatttctcttttttttttttaatttcaccttcagaatgctggtcatttgtgaccacaaatgactaccaccatctccctaaactgcatgcttccagattctaaccaggcagaaaagaactgtgcagcttctgtttttactattttcctagtatttccaccaaagttttgacagtcactcagattagaacggctaaggtcacatgtccaacactgaaccaaatacgttagccagagagatgcaatgaactgctctgtttagccgtttcacatcatcgcagggctcatgggtacctcccacgggctgagagtgggggaaagagggacagaacctcaaatgaaacacagagctgctgtcagaataaagcaaatggatgtcaagaattaacaaataatacctgaccctcctttattgaatggcactcactcatccagttccaaaacttggcatcatgtgagaccacattactctgacctctgcttccataatcacatctctttgtatgattctcttgtctacctctttcacttacaaggacccttgagattacaatggatccacacagataatacaaaacaatctccccatctcaatagtcttaatttaatcatttgtacaaggtccattttgctgtataaagtaacatgttaacatatttcagggattaggattagcacattttgaggggccattattttgcttgccacacccacatatttctttagaatcatctttagcataacctaattttagaaatgtgttctggcattatgtttattctgggttgcttctctttacttgcttaacactctgtatccttcactctagcactcaacacccactctgtccctcatgcaactttgtgagtttctcatgcaaaacaactttgatttattcatttctgagcaataatgcccaactctgtctggcacaaccaaggaaattaataattatagttctagagtcctctaaccatcaacctaaaagcttgatagtttttgatccccaaatcccaaattaatctcaaagtgttgctgagtgaatcacaatgaaattattttacatttgaaaggaatttggccaaagttcactttaccttctaaatttcaaataagccaatttgaccactgaattttagtatttaatataatgatgtgccattgttcttagtcaactaagaaacaaaacactaaaatacctttttaaaaagagtttaaaaaaaaaaaaagagcttaaaatgacttcttggtttcatctgttacaatgaagtttcaagtgcttcctgagaaagaagttctaggaagaacaactaaaaaactgtggacattacagagcaactctgaatacatgaattgacaacagtgtgccttaactttaatactctgtgtcacattgacccaaatgtaccctcctcagccagtcttcgagctctgttttctcatgggtcagagtcaattctcaaatcgtaaagcacacatccatctgcagatgcaatgggatccctaccagcatcaatgtgagccttatggctgaaagacatatcagtagtccaatcaccacccttgtacccgccctttcattggaagctctgaagcagtctccctcataagtgtgaaccttgagaagaaataatctgccaagaaggattcctcatggttaactgagctcaaattcttaatagagtctaacagccattcctgacacaagcctctcgcacactctgcatttcagggaaaagccacagactgaaatttccacctcccgaactgtgctcctgctgctgcctaagtcaaccattgtcaggaacttcctgatggaactcctgatggaacttccaaaggactgagactagtcccatccaatcagaactgtggcgttatatcctcatttgcatctatactgaccaatcagaactgattcacaacaaccaatcagaacatatgatgctgactgatcagaactgtgtgatttggatttctcatttgcataaaaatggaccaaatgggaaccagggcactaactttctctgtaaaaggccccttcccctttgtcttggtgtgcactttcggtttttcctgtttaccaactgttcagctgaataaagtttatcctctttccacacctcatattggaaacttttgttgatatgaggtaggctatggtcacaattcacaagaggacccttgaagctcagtgagatgattttcaaataacaggaggattcatccagatgattttgagaaggaagaggttactgccccaggagttcactaaggagtgaggtctgccaaaaacgtgaaaaagctgtgtagatggccctcgtgatatttcagtggaaacaatatatttcattgtaggttgaaagagtgagggtatctaacagggaggatgctgctggacagatttctccagtgtcacactaccgccaagacagcacacggagttcacaaaagacaaaatggtatggttgtgagtaggaatgcatttttcttttttttttcttcctttttttttttttttttttggtatgcatttttctgattaatcttcttggagttgctcattgtcacagcatgaaaacacctggaattcttgtttcctgacagtgcttatagatctttgatcagctatttatgttgctcaatgtccacttccatagaaaacagtagatgcagcagtctagttctcatttgctccactcatcaaattaaccaagtctgtatctatctgatgatgtgtatatatgtgtgtgtggtgcattagattcagcttgttgacaataaaacaatacttttattgactgggatactgacctacttgtatatgtgctgcctctttaaaccttccactttgtttcaatagaatagtataaaaaacaaaaagctctaggatttgc

SEQ ID NO: 354 - DNA, 3800 bp, Bat coronavirus
  source 1..3800: mol_type = other DNA
tttttcttgttttattgccactagtttctagtcagtgtgttaatctaacaactagaactcagttacctcctgcatacaccaactcatccacccgtggtgtctattaccctgacaaagttttcagatcttcagttttacatttaactcaggatttgtttttacctttcttctccaatgtgacctggttccatgctatacatgtttcagggaccaatggtattaaaaggtttgataacccagttctgccattcaacgatggcgtctattttgcttccactgagaagtctaatataataagaggatggatttttggtactaccttagattcgaagacccagtctctacttattgttaataacgctactaatgttgttattaaagtctgtgaatttcaattttgtaatgatccatttttgggtgtttattaccacaaaaacaacaaaagttggatggaaagtgagttcagagtttactctagtgcgaataattgcacttttgagtatgtctctcagccttttcttatggaccttgaaggaaaacagggtaatttcaaaaatcttagggaattcgtgtttaagaatattgatggttatttcaaaatatattctaaacatacgcctattaatttagtgcgtgatcttccccctggtttttcagctttagaaccattggtagatctgccaataggtattaacatcactaggtttcaaactttacttgctttacatagaagctatttgactcctggtgattcttcttcaggttggacagctggtgctgcagcttattatgtgggttatcttcaaccaaggacttttctactaaaatataatgagaatggaaccattacagatgctgtagactgtgcacttgaccctctttcagaaacaaagtgtacgttaaaatccttcactgttgaaaaaggaatttatcaaacctctaactttagagtccaaccaacagattctattgttagattcccaaatattacaaacttatgtccttttggtgaagtttttaacgccaccacattcgcatcagtttatgcttggaacagaaagagaattagcaactgtgttgctgattactctgtcctatataattccacttcattttctacctttaaatgttatggagtgtctcctactaaattaaatgatctctgctttactaatgtttatgcagactcatttgtgattacaggtgatgaagtcagacaaattgcgccaggacaaactggaaagattgctgactacaattataaactaccagatgattttactggttgtgttatagcttggaattctaagcatattgatgcaaaagagggcggtaattttaactatctttaccgtctctttagaaaagctaatcttaaaccctttgagagggatatctcaactgaaatttaccaagcaggcagcaaaccttgtaatggtcaaactggtctaaattgctactacccactttatagatatggattttaccctactgatggtgttggtcaccaaccttatagggtagtagtactttcttttgaacttctaaatgcaccagcaactgtttgtggacctaagaagtctactaacttggttaaaaataaatgtgtcaatttcaactttaatggtttaactggcacaggtgtcctcacagagtctaataaaaagtttctacctttccaacaatttggtagagacattgcagacactactgatgccgtccgtgatccacagacacttgagattcttgacattacaccatgttcttttggtggtgtcagtgttataacacctggaacaaatgcctctaaccaggttgctgttctttatcaggatgttaactgcacagaagtccctgttgctatccatgcagaccaacttactcccacttggcgtgtttactccacaggttctaatgtttttcaaacacgtgcaggttgtttaataggggctgaacatgtcaataactcgtatgagtgtgacatacctattggtgcaggaatatgcgccagttatcagactcaaactaattcacgtagtgtggccagtcaatctattattgcctacactatgtcacttggtgcagaaaattcagttgcttattctaataactctattgccatacctacaaattttactattagtgtgaccactgaaattctacctgtgtctatgacaaagacatcggtagactgtacaatgtatatttgtggtgattcaactgagtgcagcaaccttttgttgcaatatggtagtttttgcacacaattaaatcgtgctttaactggaatagctgttgaacaggacaaaaatactcaagaagtttttgctcaagttaaacaaatttataagacaccaccaattaaagattttggtggtttcaatttttcacaaatattaccagatccatcaaaaccaagcaagaggtcatttattgaggatttacttttcaataaagtgacacttgctgatgctggcttcatcaaacaatatggtgattgccttggtgatattgctgctagggatcttatttgtgctcaaaagttcaatggccttactgttctgccacctttgctcacagatgaaatgatcgctcaatacacttctgcactattagcaggtacaatcacttctggttggacttttggtgcaggtgctgctttacaaataccatttgccatgcaaatggcttataggtttaatggtattggagttacacagaatgttctctatgagaaccaaaaattgattgccaaccagtttaatagtgctattggcaaaattcaagactcactttcttctacagcaagtgcacttggaaaacttcaagatgttgtcaaccaaaatgcacaagctttaaacacgcttgttaaacaacttagctccaattttggagctatttctagcgtgttaaatgatatcctttcacgtctcgacaaagttgaggctgaagtgcagattgacaggttgatcacaggcagacttcaaagcttgcagacatatgtgactcaacaattaattagagctgcagaaatcagagcttctgccaatcttgctgctactaaaatgtcagagtgtgtactcggacaatcaaaaagagttgatttttgtggaaaaggctatcatcttatgtctttccctcagtcagcacctcatggtgtagtcttcttgcatgtgacatatgtccctgcacaagaaaagaacttcacaactgctcctgccatttgtcatgatggaaaagcacactttccacgtgaaggtgttttcgtttcaaatggcacacactggtttgttacacaaaggaatttttatgaaccacaaattattacaacagacaacacatttgtctctggtagctgtgatgttgtaataggaattgtcaacaacacagtttatgatcctttgcaaccagaacttgattcattcaaggaggagttggataaatactttaaaaatcatacatcacctgatgtagatttaggtgacatttctggcattaatgcttcagttgtcaatattcaaaaggaaattgaccgcctcaatgaggttgccaaaaatctaaatgaatctctcatcgatctccaagaacttggaaagtatgaacagtatataaaatggccatggtacatttggctaggttttatagctggcttgattgccataataatggtcacgattatgctttgctgtatgaccagttgctgcagttgtctcaagggctgttgttcttgcggatcttgctgcaaatttgatgaagacgactctgagccagtgctcaaaggagtcaaattacattacaca

SEQ ID NO: 355 - DNA, 3915 bp, synthetic construct
  source 1..3915: mol_type = other DNA
ttgttcactatgtattacatattgaattttcacaataatgttaagaggcaggtacaattaatacctccactttcagatgagaaaattaaggcagagaggttacataatgtgcccaaggtaccacaccttgataaacagcagctgggattctcacccatccagtcagcttcagaatctgtgcacttaactactagatgctatatagaattaatgccaaaactctcaaaatcagagtcatgagagaaaagccaaagccatcatgccaatatttgttaggttaggttaggctatgttaggttcgttttattttttattcccctaatttcctaatcttctacatttaggggaagagatgtgcttctatattcatgaatgtttatgaatgaacatcgtatgggaccattataactggaccctaaggagatatgttcttgacataattcattatcaatgatcagcattctctttgggttgattggccatgtctttatcatctccacgtcctatagaactgttcttatgaagaatatagtcaggacacacacacacatacacacacgcgcgcgcgcgatggggactcttaactagctcacccccaccaaaaagcctcatctagaagcacagagttatcatgaaatactctgggggagggggcatcatgggggtggcaattcaaagagaaatgagaaaaatcacaagatgtttaaatcaatggggatagcgctggaattttccatcctgaagattttttccagggctaaacctctgactgagttttgtttctttaacaaaggaggtggtggtggtggtagattaccttattttcaaaaacgttctttgtaaacatccaaaattatttccatgaaaattgtttctcttacatgtgacctcaattgtactcagctgaccctgtgactacttggagttgtggtggaacaaagtgcaacagtttcctcctggaagtctttcattttcattgtatgaggtgtgataaaaaaaatacagtgaatgtttaaataaaaaatttattacagtaaaagacacattaccattaattctcctcaaaatactcccccttgctttgaacacatgtatccctttgtttttgccactttctgaagcagttctggaagtcctcttttatgagtgtctttaattgtactgtagtggctgctttgatgtcctgaatcaattcaaaaagtttaccttttgtggtcattttttctttagggaagagccagcagtcgcacggtgccagatctggttaataaggcggatgaggacacaccataatgtctttagttgacagaaattgctgtataccagaagcgatgttggagcattgtcatgatagaggatgatttacagcacactgtaaaacacaccttctctcaagtgtatctcacacccaactgactgcaccaaacaagttgaaacttgtcacacatcattactaaggtttgacatgcagcttcttgtattgaatatccctgcctttccattggatggcacttagcagcaacgttcactgtattgtttaatcacacctcgtacttattctgatggagaaatttttgtcagttgagcacactttcctctctcatccttttattttctgtgtctagctttagtttgggatgagggaggacaaagtactattattatgaaattacagtggctctggaggcctctcaaatcctgactatgacacagaaaattctgaaataattcacagcaggagtactataggacttggtcagctttgcattgaacataactccacatctatattggctctgcttttgcttctcaatattaaatgctcaaatatgtcagtgctaggcactattatttatatccctctgaaacatgtttctattcaaggatgcagcattcagaagactcagtccagcgagtgacagaaaaagacttcccttggattatctatgagattgtaatagcttatctgcatatctgctcactgaatactgcctcgatcattcatatatctggctcacaatgggtaatcaataaatgtgtgatgaatggtctacaattcccagattgcagccctaacttgctcatgatggcttccagtagttttctatcaaagccacatgtggtcagtgtgcaggatgaggagtcgagcccttaaaactcaactctagaagacctactgaagcagttattacaacatgctacaatacacaaagaacaagacttgtacatcagaaacaggttgtctgaaaaagttttctattgggggaatgaagcaaattgagcctaagttttctggacaaaaagaaaaggctgatttactcagtttaagtctaagaccaaagaataagtctgagaaaaacaagatgttacctgatcttatatgcactctattattatttttgctttgcatgtcccttgtaatagtgattggttttaatgatcatttcatataaaaattaaaaagaagtacattttttaacttcctgtcaaaaattctgcctaaggtacttcctcaacacacacacgttagttgctacccctccttcaaggctctgttcatgcccgtctcctccacgaagacttttttgttctacacctagaaaggctctgcctactcaggcagttgttattacctccgatttcctactatcagatctcttcgtattatcttcttatatgactaggtctcatctccccctcaaccacaatctctctgagggctggaatattgtgcacattgccttgcacataataaaggctccagaggtatctgtctaaactggctttatttccttgagactacaagcacttattctgtgccaggcacttttaggttccagggaaaaagaggtacaaaaccagacacaaaccctaccgttatggagcttacctttttaattaaaaggtggaagggatgaacctttttttggtctctctagaaagttgcagcaggagaccataggaaatagtataaaatagttgaaagcactgtggagtgtgagtcaggataccttggtctcatctctaatttgatgtatcttgagcacatttcttaaacattggtcatctgtttccctgtatgccatataggaatcatatggttactgggaaaactgaatcagaaaacagatgcaaatcatgttggagggaactttctcaacctgataaaaagcatctatgaaaaacccacagctaacaccatacttaaaggtgaaagactggaagccttcttccgaagatcagtaacaagacaaggatgtctgctctcaccactgctattcaacattctaccggaagttctagccaggttctaagtaagaaaatgaaataaaaagaatcaagattggaaatgaagaagtactaaaactatctattttcatatgacatgaccttacttagaaaatgctaaagaatccacccccaacccccaccccaacaaaactattaaagctaataagtgaattcagcaagatttcaggatacaaggtcaatacggaaaaaaaaaagttgtatttctataaactaacaatgaacaatctgaaaatgaaattaaaaaacaacaccatttatgatagcattaaaaagaaattaaggaataaatttagcaaagaagtgtaacacttgtacgtggaaaacaacaaaacattgttgaaagaaatcaaagacctaaataaaatttttaaaatcctgcctttgtggattagaacacttaattttgttaaaatagcagtactcctcaatttgaattattcacagcaaatcctacaaaaatcttagctacctttattttcctgcagaaattgacaagctgagtttaaattttacatggaaatgcaaggaacccagaatatccaaaacaatcttgaaaaaaaggaacaaagtgggaagactcatacttcctaatttaaaaactga

SEQ ID NO: 356 - protein, 12 aa, synthetic construct
QAGILTTILTRM

SEQ ID NO: 357 - protein, 47 aa, synthetic construct
PDTKTRDNTKKKKKCPTSLMNNIDAKILNKVLANQIQQYIKKFIHHD

SEQ ID NO: 358 - protein, 70 aa, synthetic construct
VGFIPGMQGWFNICKSINVINYINKMKDKNHMIISIDAEKAFDKIQHLFMIKTLSKLGIEGKYLNIIKAI

SEQ ID NO: 359 - protein, 22 aa, synthetic construct
DKPTANIILSSEELKAFPLRSG

SEQ ID NO: 360 - protein, 24 aa, Falconid herpesvirus 1
PRRGFEPRSPVRQTGILTTILTRI

SEQ ID NO: 361 - DNA, 65 bp, Sindbis virus
  source 1..65: mol_type = other DNA
tatagtggtgagtatccccgcctgtcacgcgggagaccggggttcggttccccgacggggagcca

SEQ ID NO: 362 - protein, 163 aa, Lymphocystis disease virus
LTTVYNYSFKEKKLPETLNQAQIIPILKPGKNPLELSSYRPVSLLNCDYKILAKILASRLEKFLHVFISPDQTGYVKNRRAVYNIHRLLEIVYSSEAFESECLILTDAEKAFDFLKRSYLFETLKRFGLGENFIAWVKLIYDKPSACVWTGDRQSIFFLLKNG

SEQ ID NO: 363 - protein, 99 aa, Erythrocytic necrosis virus
PISLLNFDSEMITKLIARGRGSITLAYPDQMGFIKNWYSPDNVRRLFDVINHLNEHKTPPILLSLDAEKAFNRVEWNFLFSVLQKFHMVANFIKWVKAI

SEQ ID NO: 364 - DNA, 578 bp, synthetic construct
  source 1..578: mol_type = other DNA
aggttcatccatgtcacaaatgacaagattttgttttttatagctgagtaatattccattgtatacatataccacatcttctttgtgtattcgtctgtcagtgaactttggttacttccatatcttggctgttgtaaataatgctgcagtgaacataggggtgtgtatatcttttcgaattagtattttggatttttttcagataaatacccagaagtggaattgctgggtcatatggtaattctatttttaattttttgagggacctccatactgttttccgtagtggctgcaccaatttacaaggtgcttttctctacatccttgccaacacttgttgtttattgatttattgatgatggccattctgacacgtgacatgatagctcattgtggttttaatttgcatgtccctgatgattagtgacattgagtattttttcatatgtctattggccatctctgtgtcctctggagaaatgtctgttcaggtcctctgcccattttttaaatcagattgtttcgttttgtgtgttaagttgtatgagttccttatatattttggatattaaaccctt

SEQ ID NO: 365 - DNA, 609 bp, Severe acute respiratory syndrome coronavirus 2
  source 1..609: mol_type = other DNA
agcttcatccatgtccctacaaaggacatgaactcatcattttttatggctgcatagtattccatggtgtatatgtgccacattttcttaatccagtctatcattgctggacatttgggttggttccaagtctttgctattgtgaataatgccacaataaacataagtgtgcctgtgtctttatagcagcatgatttatagtcctttgggtatatacccagtaatgggatggctgggtcaaatggtatttctagttctagatccctgaggaatcgccacactgacttccacaatggttgaactagtttacactcccaccaacagtgtaaaagtgttcctatttctccacatcctctccagcacctgttgtttcctgactttttaatgattgccattctaactggtgtgagatggtatctcattgtggttttgatttgcatttctctgatggccagcgatggtgagcattttttcatgcgttttttggctgcataaatgtcttcttttgagaagtgtctgttcatgtccttcgcccactttttgatggggttgtttgtttttttcttgtaaatttgtttgagttctttgtagattctggatattaccctt

(19) United States
(12) Patent Application Publication
(10) Pub. No.: US 2024/0417697 A1
(43) Pub. Date: Dec. 19, 2024

(54) METHODS AND COMPOSITIONS FOR BAT IPSC PREPARATION AND USE

(71) Applicant: Icahn School of Medicine at Mount Sinai, New York, NY (US)

(72) Inventors: Thomas P. Zwaka, New York, NY (US); Marion Dejosez Zwaka, New York, NY (US); Adolfo Garcia- Sastre, New York, NY (US)

(21) Appl. No.: 18/691,516

(22) PCT Filed: Sep. 26, 2022
(86) PCT No.: PCT/US2022/077012

§ 371 Date: Mar. 13, 2024

Related U.S. Application Data
(60) Provisional application No. 63/360,472, filed on Oct. 4, 2021.
(60) Provisional application No. 63/248,835, filed on Sep. 27, 2021.

(30) Foreign Application Priority Data
Nov. 1, 2021 (GB) 2115676.5

(51) Int. Cl. C12N 5/074; A61K 39/21; A61K 39/215; C12N 7/00

(52) U.S. Cl. CPC C12N 5/0696; A61K 39/21; A61K 39/215; C12N 7/00; C12N 2501/115; C12N 2501/125; C12N 2501/235; C12N 2501/602; C12N 2501/603; C12N 2501/604; C12N 2501/606; C12N 2502/1323; C12N 2506/1307; C12N 2513/00; C12N 2740/10022; C12N 2740/10034; C12N 2740/10051; C12N 2770/20022; C12N 2770/20034; C12N 2770/20051

(57) ABSTRACT

Disclosed herein are compositions and methods of making and using bat IPSCs (BipS). Also disclosed herein are methods and compositions of virus nucleic acids residing in bat IPSCs. Also disclosed are nucleotides, cells, and methods associated with the compositions including their use as vaccines.

### CROSS-REFERENCE TO RELATED APPLICATIONS

This application claims the benefit of and priority to Great Britain Patent Application No. GB 2115676.5, filed on Nov. 1, 2021; U.S. Provisional Patent Application No. 63/360,472, filed on Oct. 4, 2020; U.S. Provisional Patent Application No. 63/248,835, filed on Sep. 27, 2021, the disclosure of each of which is hereby incorporated by reference in its entirety for all purposes.

### STATEMENT AS TO FEDERALLY SPONSORED RESEARCH

This invention was made with U.S. government support, Grant No. HR0011-19-2-0020, awarded by DARPA and Grant No. W81XWH-20-1-0270, awarded by Department of Defense (DoD), NIAID grant U19AI135972, and CRIPT (Center for Research on Influenza Pathogenesis and Response), a NIAID supported Center of Excellence for Influenza Research and Response grant CEIRR, contract #75N93019R00028. The U.S. government has certain rights to the invention.

### BACKGROUND

Bats have evolved features unique amongst mammals, including flight, laryngeal echolocation, and an immune system that shows unusual tolerance for viruses that cause life-threatening diseases in humans (e.g., SARS-CoVs, MERS-CoV, Ebola). Recent comparative genomic studies uncovered bat-specific changes to key immunity genes and exposed numerous integrated viral sequences, suggesting a particularly intimate and deep-rooted accord between bats and viruses. Still, what makes bats most distinctive is that they are home to the richest virosphere among mammals with some of the bat-related viruses causing significant outbreaks, including SARS, Ebola, and COVID-19. Remarkably, bats can be infected with viruses that are lethal to other mammals without causing any symptoms. Even more, the bat genome seems to act as a sponge for viral sequences. While endowed with a small genome, bats house a spacious number of ancient and contemporary viral insertions of retroviral and non-retroviral origin. Because some of the viral sequences are full length and even of non-bat origin, bats might supply an essential template for zoonotic viruses and act as super-spreaders. Nonetheless, how bats deal with viruses so well is poorly understood. It is clear that, although bats are a critically needed new model organism, limited access to animal and cell models has hindered their study. Bat breeding colonies are notoriously challenging to establish, and bat primary cell lines typically have a limited lifespan in vitro. Therefore, induced pluripotent stem cells would offer a research tool for bat research.

### SUMMARY

In one aspect, the disclosure provides a composition for an induced pluripotent bat stem cell (bat IPSC), wherein the cell is in a pluripotent state. In some embodiments the bat IPS cell is in a pluripotent state characterized by the expression of one or more factors for example of Klf4, Klf17, Essrb, Tfcp2l1, Tfe3, Dppa, Oct4, Sox2, Nanog, and Dusp6. In some embodiments, the IPSC cell is in a naïve pluripotent state. In some embodiments, the cell is characterized by the expression of one or more factors for example Otx2 or Zic2. In some embodiments the cell is a bat fibroblast or a bat embryonic fibroblast. In some embodiments the bat is a Rhinolophus bat or a Rhinolophus ferrumequinum bat, alternatively the bat is a Myotis bat or a Myotis myotis bat. In some embodiments, the IPS cell is capable of differentiating into embryonic bodies. In some embodiments, the embryonic bodies are capable of differentiating into three-dimensional structures comprising three germ layer markers.

In another aspect, the disclosure provides a method of producing induced pluripotent bat stem cells (bat IPSCs), the method comprising: (i) reprogramming isolated bat cells with Oct4, Sox2, cMyc, and Klf4 factors, (ii) culturing the reprogrammed cells on feeder cells in a medium comprising FGF, Leukemia inhibitory factor (Lif), SCF, and Forskolin until colonies appear; and (iii) splitting cells using a low concentration EDTA buffer; thereby producing IPSCs from bats. In some embodiments, the isolated bat cell is a fibroblast or an embryonic fibroblast. In some embodiments the cell is derived from a bat is a Rhinolophus bat or a Rhinolophus ferrumequinum bat, alternatively the bat is a Myotis bat or a Myotis myotis bet. In some embodiments, the Lif is at a concentration of 10\hat{ }4 U/ml. In some embodiments, the FGF is at a concentration of 100 ng/ml. In some embodiments, the SCF is at a concentration of 100 ng/ml. In some embodiments, the Forskolin is at a concentration of 20 nM. In some embodiments, the feeder cell is a mouse CFT mouse embryonic fibroblasts (MEF). In some embodiments, the method further comprises passaging the bat IPSCs every 5 days onto feeder cells. In some embodiments, the bat IPSC is further differentiated into embryonic bodies. In some embodiments, the embryonic bodies are further differentiated into three-dimensional structures comprising three germ layer markers.

In another aspect the disclosure provides a method of producing induced pluripotent bat stem cells (bat IPSCs), the method comprising: (i) reprogramming isolated bat cells with Oct4, Sox2, cMyc, and Klf4 factors; (ii) culturing the reprogrammed cells in feeder free medium comprising FGF, Leukemia inhibitory factor (Lif), SCF, and Forskolin until colonies appear; and (iii) splitting cells using a low concentration EDTA buffer thereby producing IPSCs from bats.

In another aspect the disclosure provides a composition for reprogramming a bat cell to produce pluripotent stem cells comprising a medium comprising FGF, Leukemia inhibitory factor (Lif), SCF, and Forskolin. In some embodiments, the Lif is at a concentration of 10\hat{ }4 U/ml. In some embodiments, the FGF is at a concentration of 100 ng/ml. In some embodiments the SCF is at a concentration of 100 ng/ml. In some embodiments, the Forskolin is at a concentration of 20 nM.

In another aspect the disclosure provides a method of obtaining viral sequences from bat IPSCs, the method comprising obtaining bat IPSCs; identifying viral sequences residing in the bat iPSC genome or intracellular virus genome; and assembling the viral sequences; thereby obtaining viral sequences from the bat iPSCs. In some embodiments, the identifying comprises sequencing the bat genome or the genome of viral particles residing in the bat IPSCs, or of viral particles shed by the bat IPSCs. In some embodiments, identifying comprises sequencing the RNA of the bat genome or the genome of viral particles residing in the bat IPSCs, or of viral particles shed by the bat IPSCs. In some embodiments, the identifying the proteins and peptides produced by the viral genome by proteomics e.g., LC-MS. In some embodiments, the method comprises translating the sequence into a protein sequence and determining whether the translated sequence has a significant homology to a known protein sequence in a viral protein database. In some embodiments, the sequence is selected from SEQ ID NO: 1-349. In some embodiments, the virus is selected from the group of a SARS-CoV-2 virus, endogenous retrovirus (RfRV), and sindbis virus. In some embodiments, the virus is a coronavirus. In some embodiments, the sequence encodes a gag protein, a pol protein, or an env protein.

In another aspect the disclosure provides a method of obtaining viral sequences from virus particles shed by bat IPSCs or cells derived from bat IPSCs, the method comprising obtaining bat IPSCs or cells derived from bat IPSCs; culturing the bat IPSCs or cells derived from bat IPSCs under conditions that allows shedding of virus particles into the culture media; collecting the culture media; identifying viral sequences residing in the culture media; and assembling the viral sequences, thereby obtaining viral sequences from virus particles shed by bat iPSCs or cells derived from bat IPSCs.

In another aspect the disclosure provides for the use of any one of the viral sequences described above for the development of a vaccine.

In another aspect the disclosure provides for a recombinant nucleic acid molecule, comprising a promoter, and a nucleic acid selected from SEQ ID NO: 1-349 encoding for a viral protein or fragment thereof. In some embodiments, a recombinant, replication deficient adenovirus, comprising nucleic acid described above is provided. In some embodiments, mRNA comprising the nucleic acid described above is provided.

In another aspect the disclosure provides for an expression vector comprising a promoter and a nucleic acid set forth in SEQ ID NO: 1-349 encoding for a viral protein or fragment thereof.

In another aspect the disclosure provides for an isolated protein or peptide comprising an amino acid sequence encoded in a nucleic acid set forth in SEQ ID NO: 1-349, wherein the peptide is no more than 100 amino acids in length, and an optional pharmaceutically acceptable carrier. In some embodiments, the protein or peptide is no more than 30 amino acids in length or 20 amino acids in length. In some embodiments, the protein or peptide is synthetic.

In another aspect the disclosure provides for a pharmaceutical composition comprising the adenovirus of described above, the mRNA described above, or the protein or peptide of any described above and a pharmaceutically acceptable carrier or excipient. In some embodiments, the pharmaceutical composition comprises a plurality of (e.g., 2, 3, 4, 5, 6, 7, 8, 9, 10, or more) proteins or peptides described above and a pharmaceutically acceptable carrier or excipient. In some embodiments, the pharmaceutical composition comprises a nucleic acid encoding the mRNA described above or the protein or peptide described above and a pharmaceutically acceptable carrier or excipient. In some embodiments, the pharmaceutical composition comprises one or more nucleic acids encoding a plurality of (e.g., 2, 3, 4, 5, 6, 7, 8, 9, 10, or more) mRNAs of described above or proteins or peptides of described above, and a pharmaceutically acceptable carrier or excipient. In some embodiments, the pharmaceutical composition further comprises a liposome, wherein the protein or peptide or the nucleic acid encoding the protein or peptide is disposed within the liposome. In some embodiments, the pharmaceutical composition further comprises a lipid nanoparticle, wherein the protein or peptide or the nucleic acid encoding the protein or peptide is disposed within the lipid nanoparticle. In some embodiments, the pharmaceutical composition comprises an immunogenicity enhancing adjuvant.

In another aspect the disclosure provides for a vaccine that stimulates a T cell mediated immune response when administered to a subject, the vaccine comprising the pharmaceutical composition described above. In some embodiments, the vaccine is a priming vaccine and/or a booster vaccine.

In another aspect the disclosure provides for a recombinant cell comprising a nucleic acid or a portion of a nucleic acid set forth in SEQ ID NO: 1-349. In some embodiments, the recombinant cell comprises a protein or a portion of a protein encoded by a nucleic acid set forth in SEQ ID NO: 1-349.

In another aspect the disclosure provides for a composition comprising an inhibitor of a protein encoded by a nucleic acid selected from SEQ ID NO: 1-349.

For a fuller understanding of the nature and advantages of the present disclosure, reference should be had to the ensuing detailed description taken in conjunction with the accompanying figures. The present disclosure is capable of modification in various respects without departing from the present disclosure. Accordingly, the figures and description of these embodiments are not restrictive.

### DESCRIPTION OF THE DRAWINGS

These and other features, aspects, and advantages of the present disclosure will become better understood with regard to the following description, and accompanying drawings, where:

FIG. 1A-FIG. 1I illustrate the derivation of pluripotent bat stem cells. FIG. 1A, illustrates the bat pluripotent stem cell derivation strategy. BEF, embryonic fibroblasts; OSMK, Oct4, Sox2, cMyc, Klf4; FB, fibroblast medium; PSC, pluripotent stem cell medium; PSC+, PSC with additives, FIG. 1B, shows exemplary morphologies of established BiPS cell colonies grown on mouse embryonic fibroblasts. FIG. 1C, Immunofluorescent detection of Oct4 in BiPS cells. FIG. 1D, MA plot of RNA-seq data illustrating the transcriptional differences between bat embryonic fibroblast (BEF) and pluripotent stem cells (BiPS). Selected genes with known functions in the establishment or maintenance of pluripotency are highlighted in dark filled circles. FIG. 1E, shows a Kmean cluster analysis of ATAC-seq signals obtained from BEF or BiPS cells. C, cluster. FIG. 1F, shows a density plot of RRBS results obtained from BEF and BiPS cells. PCC, Pearson correlation coefficient. FIG. 1G, shows scatter plots of histone 3 methylation status at K4 (activating chromatin modification) or K27 (repressing chromatin modification) after ChIP-seq from BEF or BiPS cells as indicated. FIG. 1H, shows a scatter plot of H3K4me3 and H3K27me3 in BiPS cells illustrating the occurrence of bivalent chromatin sites in BiPS cells. FIG. 1I, shows RNA-seq, ATAC-seq and H3K4me3 or H3K27me3 ChIP-seq signals of selected genes with known roles in reprogramming that are activated (Nanog, Kit) or repressed (Thy1) in BiPS when compared to BEF cells.

FIG. 2A-FIG. 2M. illustrate the characterization of pluripotent stem cells generated from Rhinolophus ferrumequinum and Myotis myotis fibroblasts. FIG. 2A, shows exemplary microscopic images of human embryonic stem cells (H9)(lower panels) and bat pluripotent stem cells (upper panel) at indicated magnifications showing cytoplasmic vesicles. FIG. 2B, shows a karyotype analysis of BiPS cells at passage 17. Shown is a representative image after Giemsa staining of a metaphase spread with 56 chromosomes.

FIG. 2C, shows PCR verification of reprograming-associated virus clearing. Bat iPS cells (BiPS) at passage 92 were tested for Sendai virus clearance in comparison to the embryonic fibroblasts used as starting material (BEF), adult fibroblasts as negative control (NC), and freshly-transduced cells at passage 3 as a positive control (PC). bp, base pairs; SeV, Sendai virus; KOS, KLF4-OCT4-SOX2, FIG. 2D, shows a correlation scatter plot of methylation level at common CpG sites in duplicate samples of BEF or BiPS cells. BEF, bat embryonic fibroblast cells; BiPS, bat pluripotent stem cells; PCC, Pearson correlation coefficient. FIG. 2E Venn diagram illustrating the overlap of bivalent genes in bat iPSCs and human ES cells. FIG. 2F, Correlation plot of shrunken log 2-fold changes in ATAC-seq signal with log 2-fold expression changes. Shown are all values with p<0.05. FIG. 2G, Correlation of log 2-fold changes in H3K4 trimethyla-tion (H3K4me3, left) or H3K27 trimethylation (H3K27me3, right) with log 2-fold changes in gene expression. FIG. 2H, Correlation of log 2-fold gene expression changes with the difference in the methylated fraction of promoters (left) or gene bodies (right) fractions. FIG. 2I, Characterization of Myotis myotis induced pluripotent stem cells. Microscopic images of Myotis myotis iPS cells after immunostaining to detect pluripotency marker Oct4. FIG. 2J, Microscopic images of Myotis myotis iPS cells that underwent differentiation and immunostaining to detect Pax6, Brachyury (T) and Afp as markers of ectoderm, mesoderm and endodem, respectively. FIG. 2K-FIG. 2M illustrate the characterization of pluripotency markers in pluripotent stem cells generated from Rhinolophus ferrumequinum fibroblasts FIG. 2K, Sequencing tracks showing expression, ATAC-seq signal, Histone H3K27 trimethylation (H3K27me3) and Histone H3K4 trimethylation (H3K4me3) status of pluripotency markers Oct4, and Sox2 in bat embryonic fibroblasts (BEF) or induced pluripotent stem cells (BiPS). FIG. 2L, Fraction of methylated sites in promoters of pluripotency genes that did show promoter methylation. FIG. 2M, Immunofluorescence images of bat pluripotent stem cells after staining of markers of naïve (Tfe3 and Tfcp2l1) or primed pluripotency (Zic2 and Otx2).

FIG. 3A-FIG. 3G illustrate the differentiation potential of bat pluripotent stem cells. FIG. 3A, illustrates exemplary immunofluorescence microscopy images after staining with antibodies detecting the expression of lineage-specific markers Pax6, Afp or Brachyury (T) following specific directed differentiation into ectoderm, endoderm or mesoderm, respectively. FIG. 3B illustrates exemplary immunofluorescence images of embryonic bodies (EB) that formed after 3D-differentiation of BiPS cells and were stained with antibodies to detect markers specific to all three germ layers as in FIG. 3A. FIG. 3C shows RNA-seq signal of selected lineage-specific marker genes in BiPS cells that underwent monolayer differentiation as in (FIG. 3A) or embryonic body differentiation as in (FIG. 3B). EB, embryonic body differentiation, EC, human ectoderm differentiation protocol; EN, human endoderm differentiation protocol; M, human mesoderm differentiation protocol. FIG. 3D, illustrates exemplary microscopic images of Hematoxylin-Eosin-stained sections of tumor tissue after injection of BiPS cells into immunocompromised mice exhibiting ectodermal (left), mesodermal (middle) and endodermal (right) features. FIG. 3E shows exemplary images of floating blastoids that were obtained from BiPS cells after exposure to Bmp4 to capture their morphology by phase-contrast microscopy (left) and to detect Oct4 expression in inner-cell mass-like cell clusters by after immunofluorescence staining (middle, right). FIG. 3F illustrates Phase-contrast microscopy image of atypical blastocyst outgrowth-like cell cluster that formed after attachment of blastoids to the cell culture vessel surface during Bmp4-induced differentiation as in FIG. 3E. ICL, Inner cell mass-like; TLO, trophoblast-like outgrowth. FIG. 3G shows an expression profile of genes associated with tumor suppression. The data sets were from this study (bat), GSE53212 (mouse, GEO), PRJNA400257 (Naked mole-rat, BioProject), and GEOGSE175070 (human, GEO). ARF, ADP ribosylation factor; BEF, bat embryonic fibroblasts; BiPS, bat induced pluripotent stem cells, ERAS, ES cell-expressed Ras; FOXO6, Forkhead Box 06; H9, human ES cells; HAS, Hyaloron-synthase; MEFs, mouse embryonic fibroblasts; NMR, naked mole-rat.

FIG. 4A-FIG. 4D. illustrate the differentiation potential of bat pluripotent stem cells. FIG. 4A, Schematic of differentiation strategies. FIG. 4B, Representative image of embryoid bodies differentiated for 3 days. FIG. 4C, shows a MA plot depicting the log 2 mean expression and log 2 fold expression changes of all genes in bat pluripotent stem cells (BiPS) after exposure to the noted differentiation conditions illustrated in FIG. 4A. EB, Embryoid body differentiation; EC, human ectoderm differentiation conditions; EN, human endoderm differentiation conditions; M, human mesoderm differentiation conditions. FIG. 4D, shows a heatmap depicting expression changes of genes known as markers for human ectoderm, mesoderm, or endoderm during the differentiation of BiPS under the conditions described in FIG. 4A.

FIG. 5A-5D. illustrate distinct characteristics of pluripotent bat stem cells. FIG. 5A shows principal component analysis of induced pluripotent bat stem cells (BiPS) in comparison to those derived from other species, b, human; m, mouse. PS, pluripotent stem cells, iPS, induced pluripotent stem cells, S, embryonic stem cells, EF, embryonic fibroblasts. FIG. 5B shows a plot of genes that contribute to the differences of pluripotent bat and mouse stein cells as part of principal component 1 (PC1). Highlighted in light blue is the “leading edge” comprised of the top 5% of PC1-contributing genes. FIG. 5C shows selected GO and FIG. 5D shows KEGG pathways identified to be significantly enriched among the top 5% of PC1-contributing genes/leading edge genes defined in (FIG. 5B) were plotted by their odds ratio, with the color of each circle indicating the enrichment p-value and the size indicating the number of genes present in the respective category. ER, endoplasmic reticulum: PT, protein targeting: Pos, positive; Reg, regulation.

FIG. 6A illustrates the interaction of genes that are part of the KEGG Corona Virus Disease pathway. Nodes are colored based on the log 2 fold change between BiPS and mouse iPS cells. Red indicates genes that are expressed at a higher level in BiPS, blue indicates those that are expressed at a lower level. Bold borders indicate proteins that were present in the top 5% of genes in PC1 (leading edge). FIG. 6B illustrates that the selection analyses of leading edge-genes by comparative genomics analyses of the R. ferrumequinum lineage identified eight genes showing significant evidence of positive selection. Additional lineages and the number of genes showings selection found in them, are highlighted in brackets.

FIG. 7A-7J illustrate viral tolerance of pluripotent bat stem cells. FIG. 7A shows the expression of indicated ERV elements in bat embryonic fibroblasts (BEF) and iPS cells (BiPS) as determined by extracting the overlap between RNA-seq reads mapped to the R. ferrumequinum genome and known mapped ERV elements. Shown are the elements with the most evident differences. FIG. 7B, shows an exemplary electron microscopy image of cytoplasmic vesicles of BiPS cells containing virus-like structures. Bottom: higher magnification of viroid structures: Intracellular inclusions of virus-like particles (black arrows) with granular and electron-dense content (white arrowheads), typically surrounded by double membrane structures (white arrows), and some of them coated with protrusions (black arrowheads). FIG. 7C, Western blotting in human 293FT (kidney tumor cell line) and embryonic stem cells (H9), mouse 3T3 (fibroblasts) and embryonic stem cells (R1), and bat pluripotent stem cells (BiPS) with a HERV K capsid (Cap) specific antibody detecting human endogenous retroviruses. FIG. 7D, shows exemplary immunofluorescence images of BiPS cells detecting the HERVK Gag/Cap protein. FIG. 7E, shows Western blotting in human 293FT, H9, mouse 3T3 and R1, and BiPS with a pan coronavirus antibody known to be specific for the nucleocapsid; its reactivity includes but might not be limited to feline infectious peritonitis virus type 1 and 2, the canine coronavirus (CCV), pig coronavirus transmissible gastroenteritis virus (TGEV), and ferret coronavirus. FIG. 7F, illustrates exemplary immunofluorescence images of BiPS cells after detection of pan coronavirus antigen. FIG. 7G, shows exemplary immunofluorescence images of BiPS cells after detection of double stranded RNA characteristic RNA viruses.

FIG. 8A-FIG. 8C illustrate exemplary microscopic images of bat pluripotent stem cells. FIG. 8A, shows a 40× magnification of a bat pluripotent stem cell colony. FIG. 8B and FIG. 8C show an overview of transmission electron microscopy of bat pluripotent stem cells. Vi, vesicles containing viral-like structures; OV, other vesicle structures filled with homogenous content: Nu, Nucleus; A, autophagosome; M, mitochondria. FIG. 8D shows a higher magnification of the structures.

FIG. 9A-9H illustrate exemplary virome mining in BIPS cells. FIG. 9A flow diagram of the sequence mining for viral sequences in the bat genome. FIG. 9B shows the taxonomic distribution of virome reads as determined by the metagenomic classifier Kraken2. The distribution of the reads that were mapped according to the virus data base are shown in a phylogenetic tree. The green color coding represents the number of taxa observed, the red nodes denote particular taxa of interest. FIG. 9B shows the number of viral species as classified by Kraken through RNA-seq and iso-seq sequencing. FIG. 9C shows the number of individual viruses species and subspecies obtained from iso-seq (top panel) and RNA-seq (bottom panel). FIG. 9D shows RNA and Iso-seq sequencing tracks for a newly discovered full-length retrovirus sequence, RFe-V-MD1, aligned to the R. ferrumequinum genome. The Iso-seq fragment represents a 6088 bp-long transcript. FIG. 9E shows genomic and sequence track for short integrated viral sequences for Columbid/Falconid herpesvirus and Sindbis virus. FIG. 9F illustrate the short viral insertion shown in FIG. 9E form stem-loop structures. FIG. 9G illustrates another example of a short viral integration showing homology to two human herpesvirus 4 isolates (HKD40 and HKNPC60), the human respiratory syncytial virus (Kilifi isolate), and a fragment of about 500 bp that was identified at the end of a SARS-CoV2 isolate in an infected patient (OU077605.1). FIG. 9H shows a genome track for a Scotophilus bat coronavirus 512 homologous sequence of the spike protein coding region. FIG. 9I ImageStream analysis after immunofluorescence staining of BiPS cells. A brightfield image, Crystal Violet nuclear staining (Nucleus), dsRNA staining (dsRNA) and an overlay is shown for each representative cell.

FIG. 10A shows exemplary results of long-read RNA sequencing (iso-seq), the sequencing reads were mapped against a virus database, using a metagenomic classification tool (Kraken) including viruses from several significant viral families, including Paramyxoviridae, Rhabdoviridae, Filoviridae, Bornaviridae, Flaviviridae, Coronaviridae, Picornaviridae, and Retroviridae. FIG. 10B shows the number of viral species as classified in BEFs and BiPS. FIG. 10C illustrates an exemplary assembly of full-length viruses, shorter viral insertions, and novel, more distant viruses based on the sequencing data from BiPS cells such as the shown full-length bat retrovirus (RFeRV). The top shows short nucleotide reads aligned to a full length sequence. The middle and lower prat of the figure shows the position of a Gag, Pol, and Env protein in the genome.

FIG. 11A-11D illustrate exemplary protein and nucleotide sequences identified in the BiPS cells that are associated with viruses. FIG. 11A shows a protein sequence with homology to a hypothetical protein CoVHLJ_8—from Columbid alphaherpesvirus 1 and a nucleotide sequence that is similar to a Sindbis virus defective interfering particle di-2. FIG. 11A discloses SEQ ID NOS 8, 356, 360, 9 and 361, respectively, in order of appearance. FIG. 11B shows a protein or a protein fragment with homologies to an RNA-dependent DNA polymerase of the lymphocystis disease virus and of the erythrocytic necrosis virus. FIG. 11B discloses SEQ ID NOS 15, 357-359, 362, 14, 358 and 363, respectively, in order of appearance. FIG. 11C illustrates the results of mapping of a region residing in the first intron of the XPA gene (a DNA damage and repair factor) on chromosome 12. A BLAST search with the fragment showed homology to two human herpesvirus 4 isolates (HKD40 and HKNPC60), the human respiratory syncytial virus (Kilifi isolate), and a fragment of about 500 bp that was identified at the end of a SARS-CoV2 isolate in an infected patient. FIG. 11C discloses SEQ ID NOS 364 and 365, respectively, in order of appearance. FIG. 11D shows a phylogenic analysis of the genomic sequences mostly resembled the spike protein-encoding genomic portion of human coronavirus 229E and the human coronavirus OC43.

### DETAILED DESCRIPTION

Various features and aspects of the disclosure are discussed in more detail below.

The disclosure is based, in part, upon the discovery that induced pluripotent bat stem cells can be produced and are stable in culture, readily differentiate into all three germ layers, and form complex embryoid bodies, including organoids. Bat iPSCs (BiPS) and their differentiated progeny can be used for example as an accessible and versatile tool required to advance bats as a new model system. Further, BiPS can provide the platform to further understand the role bats play as virus reservoirs and enable new insights into emerging viruses, such as SARS-CoV-2, and better prepare for future pandemics. BiPS can enable studies that directly impact every aspect of bats' particular biology, including this mammal's unique adaptations of flight, echolocation, extreme longevity, and unique immunity. Further, BiPS are also useful for example in understanding of bats' asymptomatic response to viral pathogens.

Accordingly, the disclosure provides BiPS, methods of producing and using BiPS, and compositions for reprogramming bat cells.

In another aspect, the disclosure is based in part on the discovery of viruses and viral nucleic acids and proteins in BiPS. The viruses, viral nucleic acids, viral proteins, viral nucleic acid sequences, and protein sequences are useful in the development of therapeutics and prophylactics for viral diseases, such as vaccines, antibodies, and small molecule antivirals.

Accordingly, the disclosure provides viral nucleic acid and protein sequences, expression constructs, vectors comprising the expression constructs, methods of making and using therapeutics and prophylactics against viral diseases such as vaccines, antibodies, and small molecule antivirals.

Unless otherwise defined herein, scientific and technical terms used in this application shall have the meanings that are commonly understood by those of ordinary skill in the art.

Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J.B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, NY (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, NY (1998); Coligan et al., Short Protocols in Protein Science, John Wiley & Sons, NY (2003); Short Protocols in Molecular Biology (Wiley and Sons, 1999).

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

Throughout this specification and embodiments, the word “comprise,” or variations such as “comprises” or “comprising,” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in an embodiment of the disclosure.

Exemplary methods and materials are described herein, although methods and materials similar or equivalent to those described herein can also be used in the practice or testing of the present disclosure. The materials, methods, and examples are illustrative only and not intended to be limiting.

### I. Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. The section headings used herein are for organizational purposes only and are not to be construed as limiting the subject matter described.

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, “residue” refers to a position in a protein and its associated amino acid identity.

As used herein the term “antigen” is a substance that induces an immune response. An antigen can be a neoantigen.

As used herein the term “antigen-based vaccine” is a vaccine composition based on one or more antigens, e.g., a plurality of antigens. The vaccines can be nucleotide-based (e.g., virally based, RNA based, or DNA based), protein-based (e.g., peptide based), or a combination thereof.

As used herein the term “coding region” is the portion(s) of a gene that encode protein.

As used herein the term “coding mutation” is a mutation occurring in a coding region.

As used herein the term “ORF” means open reading frame.

As used herein the term “epitope” is the specific portion of an antigen typically bound by an antibody or T cell receptor.

As used herein the term “immunogenic” is the ability to elicit an immune response, e.g., via T cells, B cells, or both.

As used herein the term “HLA binding affinity” “MHC binding affinity” means affinity of binding between a specific antigen and a specific MHC allele.

As used herein the term “ELISPOT” means Enzyme-linked immunosorbent spot assay—which is a common method for monitoring immune responses in humans and animals.

The term “lipid” includes hydrophobic and/or amphiphilic molecules. Lipids can be cationic, anionic, or neutral. Lipids can be synthetic or naturally derived, and in some instances biodegradable. Lipids can include cholesterol, phospholipids, lipid conjugates including, but not limited to, polyethylenegly col (PEG) conjugates (PEGylated lipids), waxes, oils, glycerides, fats, and fat-soluble vitamins. Lipids can also include dilinoleylmethyl-4-dimethylaminobutyrate (MC3) and MC3-like molecules.

The term “lipid nanoparticle” or “LNP” includes vesicle like structures formed using a lipid containing membrane surrounding an aqueous interior, also referred to as liposomes. Lipid nanoparticles includes lipid-based compositions with a solid lipid core stabilized by a surfactant. The core lipids can be fatty acids, acylglycerols, waxes, and mixtures of these surfactants. Biological membrane lipids such as phospholipids, sphingomyelins, bile salts (sodium taurocholate), and sterols (cholesterol) can be utilized as stabilizers. Lipid nanoparticles can be formed using defined ratios of different lipid molecules, including, but not limited to, defined ratios of one or more cationic, anionic, or neutral lipids. Lipid nanoparticles can encapsulate molecules within an outer-membrane shell and subsequently can be contacted with target cells to deliver the encapsulated molecules to the host cell cytosol. Lipid nanoparticles can be modified or functionalized with non-lipid molecules, including on their surface. Lipid nanoparticles can be single-layered (unilamellar) or multi-layered (multilamellar). Lipid nanoparticles can be complexed with nucleic acid. Unilamellar lipid nanoparticles can be complexed with nucleic acid, wherein the nucleic acid is in the aqueous interior. Multilamellar lipid nanoparticles can be complexed with nucleic acid, wherein the nucleic acid is in the aqueous interior or and/or can be sandwiched between the layers.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methylphosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NRi (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The terms “polypeptide,” “oligopeptide,” “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably generally to refer to a combination of nucleic acid sequences or elements that are expressed together or are operably linked for expression. In some cases, an expression cassette refers to the combination of regulatory elements and a gene or genes to which they are operably linked for expression.

As used herein, the term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

The term “sequence similarity,” in all its grammatical forms, refers to the degree of identity or correspondence between nucleic acid or amino acid sequences that may or may not share a common evolutionary origin.

“Percent (%) sequence identity” or “percent (%) identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Alternatively, sequence similarity or dissimilarity can be established by the combined presence or absence of particular nucleotides, or, for translated sequences, amino acids at selected sequence positions (e.g., sequence motifs).

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions.

However, in common usage and in the instant application, the term “homologous,” when modified with an adverb such as “highly,” may refer to sequence similarity and may or may not relate to a common evolutionary origin.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as miRNA, siRNA, or shRNA.

As used herein, “isolated molecule” (where the molecule is, for example, a polypeptide, a polynucleotide, or fragment thereof) is a molecule that by virtue of its origin or source of derivation (1) is not associated with one or more naturally associated components that accompany it in its native state, (2) is substantially free of one or more other molecules from the same species (3) is expressed by a cell from a different species, or (4) does not occur in nature.

The term “subject” encompasses a cell, tissue, or organism, human or non-human, whether in vivo, ex vivo, or in vitro, male or female. The term subject is inclusive of mammals including humans.

The term “mammal” encompasses both humans and non-humans and includes but is not limited to humans, non-human primates, canines, felines, murines, bovines, equines, pteropines, and porcines.

As used herein, a “vector,” refers to a recombinant plasmid or virus that comprises a nucleic acid to be delivered into a host cell, either in vitro or in vivo. A “recombinant viral vector” refers to a recombinant polynucleotide vector comprising one or more heterologous sequences (i.e. a nucleic acid sequence not of viral origin). In the case of recombinant AAV vectors, the recombinant nucleic acid is flanked by at least one inverted terminal repeat sequence (ITR). In some embodiments, the recombinant nucleic acid is flanked by two ITRs.

The phrase “pharmaceutical composition” refers to a mixture containing a specified amount of a therapeutic, e.g., a therapeutically effective amount, of a therapeutic compound in a pharmaceutically acceptable carrier to be administered to a mammal, e.g., a human, in order to treat a disease.

The phrase “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio.

Each embodiment described herein may be used individually or in combination with any other embodiment described herein.

### II. Bat Pluripotent Stem Cells (BiPS)

The disclosure is based, in part, upon the discovery that bat induced pluripotent stem cells (iPSC) (BiPS) can be produced and are stable in culture, proliferate, readily differentiate into all three germ layers, and form complex embryoid bodies, including organoids.

Accordingly, compositions and methods of making and using the BiPS are provided herein.

### BiPS of the Disclosure

In some embodiments, BiPS are provided. In some embodiments the pluripotent state of the BiPS is characterized by the expression of one or more factors selected from the group of Klf4, Klf17, Essrb, Tfcp2l1, Tfe3, Dppa, Oct4, Sox2, Nanog, and Dusp6. In some embodiments, 1, 2, 3, 4, 5, 6, 7, 8, 9, or all 10 factors are expressed in the BiPS. Pluripotent stem cells can be classified into at least naïve and primed stem cell states based on the growth characteristics in vitro and their potential rise to all somatic lineages and the germ line in chimeras. In some embodiments, the BiPS are in a naïve pluripotent state. In some embodiments, the BiPS are further characterized by the expression pf one or more factors for example Otx2 or Zic2.

Bats are divided in two groups: fruit-eating megabats, and the echolocating microbats. Megabats are further divided into Yinpterochiroptera that include the Pteropodidae, or megabat family, as well as the family of Rhinolophoidea, and Yangochiroptera. Rhinolophoidea can be further divided into Hipposideridae, Craseonycteridae, Megadermatidae, Rhinopomatidae and Rhinolophidae. In some embodiments, the BiPS can be derived from isolated source bat cells from embryonic, young, or adult bats. In some embodiments, the bat is a Rhinolophus bat. In some embodiments the bat is a wild horseshoe bat (Rhinolophus ferrumequinum). In some embodiments, the bat is a Myotis bat or a Myotis myotis bat. In some embodiments, embryonic fibroblasts (BEF) cells can be isolated from the bat. In some embodiments, adult fibroblasts cells can be isolated from the bat.

A BiPS of the disclosure may be isolated, substantially isolated, purified or substantially purified. The iPSC is isolated or purified if it is completely free of any other components, such as culture medium, other cells of the disclosure or other cell types. The iPSC is substantially isolated if it is mixed with carriers or diluents, such as culture medium, which will not interfere with its intended use. Alternatively, the iPSC of the disclosure may be present in a growth matrix or immobilized on a surface as discussed below.

In some embodiments, the BiPS are further differentiated into embryonic bodies. In some embodiments, the BiPS can be further differentiated into endoderm (Afp+), ectoderm (Tbxt+), and mesoderm (Pax6+). The embryonic bodies derived from the BiPS can be further differentiated into three-dimensional structures comprising the three germ layer markers.

Techniques for producing and culturing iPSCs are well known to a person skilled in the art. Suitable conditions are discussed below.

### Method of Producing an BiPS of the Disclosure

The one aspect, the disclosure also provides a method of producing a population of BiPS, comprising culturing source bat cells under conditions which reprogram the source bat cells to produce the BiPS. Any of the source bat cells discussed above may be used.

Induced pluripotent stem cells (iPSCs) are a type of pluripotent stem cell that can be generated (reprogrammed) from a non-pluripotent cell of a multicellular organism, such as a somatic cell. iPSCs are characterized in that they propagate indefinitely and can differentiate into the three germ layers endoderm, mesoderm and ectoderm, form embryonic bodies, develop into teratomas in vivo, and can form fully differentiated tissues including but not limited to neurons, cardiomyocytes, hepatocytes, and immune cells. Typically, iPSCs express a group of markers for stem cells on the surface of the cell such as SSEA-4, TRA-1-60, and CD30, though expressed markers and timing of expression for the markers can vary (for example as described in Pomeroy et al., Stem Cells Transl Med. (2016) 5(7): 870-882). Recently, two protocols to produce bat reprogrammed stem cells were published (Mo et al., Theriogenology (2014)15; 82(2):283-93, Aurine et al., BioRxiv (2019)). However, neither of the protocols provides for BiPS that are able to differentiate into the three germ layers or form embryonic bodies or teratomas in vivo. Thus, lack of access to robust cell models has hindered further understanding of bat asymptomatic response to viral pathogens.

To establish bats as new model study species, initially the Yamanaka reprogramming protocol based on four reprogramming factors (Oct4, Sox2, Klf4, and cMyc) (Takahashi K. et al., Cell (2006) 25; 126(4):663-76, and. Hochedlinger K. et al., Cold Spring Harb Perspect Biol. (2015) 7(12): a019448), that is highly effective in mice, humans, and other mammalian species (e.g., dog, pig, marmoset) was tried to produce induced pluripotent stem cells (iPSCs) from a wild horseshoe bat (Rhinolophus ferrumequinum). However, the protocol failed to produce BiPS that were stable in culture, and that proliferated. Though the protocols failed, the Yamanaka factors triggered the formation of rudimentary stem cell-like colonies even though they ceased to expand.

Here, methods of making BiPS are provided that overcome these problems.

The method preferably comprises culturing the source bat cells with a Sendai virus system, a retroviral system, a lentiviral system, microRNA or other reprogramming factors which is/are capable of reprogramming the source bat cells to produce the BiPS. In some embodiments, the method of making bat iPSCs comprises (i) reprogramming isolated bat cells with Oct4, Sox2, cMyc, and Klf4 factors; (ii) culturing the reprogrammed cells in a medium comprising FGF, Leukemia inhibitory factor (Lif), SCF, and Forskolin until colonies appear; and (iii) splitting cells using a low concentration EDTA buffer.

In some embodiments, the reprogramming factors can be delivered to the bat cells with viruses such as a Sendai virus, retrovirus, AAV, nonviral vector systems, physical delivery, mechanical and chemical methods, or with mRNA delivery. In some embodiments, the reprogramming factors comprise Oct4, Sox2, cMyc, and Klf4 factors. In some embodiments, the reprogramming factors comprise additional factors.

In some embodiments, the method comprises culturing the cells in a feeder free medium. In some embodiments, the cells can be cultured on feeder cells, such as CFT mouse embryonic fibroblasts.

In some embodiments, the feeder cell free or the feeder cell culture medium comprises FGF, Leukemia inhibitory factor (Lif), SCF, and Forskolin. In some embodiments, the Lif is at a concentration of 10\hat{ }4 U/ml. In some embodiments, the FGF is at a concentration of 100 ng/ml. In some embodiments, the SCF is at a concentration of 100 ng/ml. In some embodiments, the Forskolin is at a concentration of 20 nM. In some embodiments, the Lif is at a concentration of 10\hat{ }4 U/ml, the FGF is at a concentration of 100 ng/ml, the SCF is at a concentration of 100 ng/ml and the Forskolin is at a concentration of 20 nM. In some embodiments, the Lif is at a concentration of 10\hat{ }4 to 10≡U/ml. In some embodiments, the FGF is at a concentration of 100 ng/ml. In some embodiments, the SCF is at a concentration of 10-100 ng/ml. In some embodiments, the Forskolin is at a concentration of 5-20 nM. In some embodiments, the Lif is at a concentration of 10\hat{ }4 to 10≡U/ml, the FGF is at a concentration of 4-100 ng/ml, the SCF is at a concentration of 10-100 ng/ml and the Forskolin is at a concentration of 5-20 nM. In some embodiments, the concentration of Lif is 40%, 30%, 20%, 10%, or 5% more or less than 10\hat{ }4 U/ml. In some embodiments, the concentration of FGF is 40%, 30%, 20%, 10%, or 5% more or less than 100 ng/ml. In some embodiments, the concentration of SCF is 40%, 30%, 20%, 10%, or 5% more or less than 100 ng/ml. In some embodiments, the concentration of Forskolin is 40%, 30%, 20%, 10%, or 5% more or less than 20 nM. In some embodiments, the concentration of Lif is about 10\hat{ }4 U/ml. In some embodiments, the concentration of FGF is about 100 ng/ml. In some embodiments, the concentration of SCF is about 100 ng/ml. In some embodiments, the concentration of Forskolin is about 20 nM.

In some embodiments, the BiPS are passaged, i.e. moved into fresh media. In some embodiments the BiPS are passaged every 1, 2, 3, 4, 5, 6, 7, 8, 9, or 10 days. In some embodiments, the BiPS are passaged every 5 days. In some embodiments, the BiPS are passaged when they are 50%, 60%, 70%, 80%, 90%, or 100% confluent. In some embodiments, the BiPS are passaged before they are confluent. In some embodiments, the feeder cells are freshly changed every passage. In some embodiments, the feeder cells are irradiated. In some embodiments, the BiPS are passaged using a low concentration EDTA buffer. In some embodiments, the BiPS are passaged using a low concentration EDTA buffer with a EDTA concentration less than 0.48 mM EDTA. In some embodiments the BiPS can be passaged indefinitely. In some embodiments the BiPS can be passaged at least to passage 78.

In some embodiments, the BiPS are further differentiated into embryonic bodies. In some embodiments, the BiPS can be further differentiated into endoderm (Afp+), ectoderm (Tbxt+), and mesoderm (Pax6+). The embryonic bodies can be further differentiated into three-dimensional structures comprising the three germ layer markers.

In some embodiments, a medium is provided that is conducive to producing and maintaining BiPS comprising FGF, Leukemia inhibitory factor (Lif), SCF, and Forskolin. In some embodiments, the medium comprises FGF at a concentration of 20 nM, Leukemia inhibitory factor (Lif) at a concentration of 10\hat{ }4 U/ml, SCF at a concentration of 100 ng/ml, and Forskolin at a concentration of 100 ng/ml. In some embodiments, the Lif is at a concentration of 10\hat{ }4 U/ml, the FGF is at a concentration of 100 ng/ml, the SCF is at a concentration of 100 ng/ml and the Forskolin is at a concentration of 20 nM. In some embodiments, the Lif is at a concentration of 10\hat{ }4 to 10≡U/ml. In some embodiments, the FGF is at a concentration of 100 ng/ml. In some embodiments, the SCF is at a concentration of 10-100 ng/ml. In some embodiments, the Forskolin is at a concentration of 5-20 nM. In some embodiments, the Lif is at a concentration of 10\hat{ }4 to 10≡U/ml, the FGF is at a concentration of 4-100 ng/ml, the SCF is at a concentration of 10-100 ng/ml and the Forskolin is at a concentration of 5-20 nM. In some embodiments, the medium comprises FGF at a concentration of 40%, 30%, 20%, 10%, or 5% more or less than 20 nM, Leukemia inhibitory factor (Lif) at a concentration of 40%, 30%, 20%, 10%, or 5% more or less than 10\hat{ }4 U/ml, SCF at a concentration of 40%, 30%, 20%, 10%, or 5% more or less than 100 ng/ml, and Forskolin at a concentration of 40%, 30%, 20%, 10%, or 5% more or less than 100 ng/ml.

An important method for reprogramming is the use of messenger RNA specific for the reprogramming factors since this does not involve any genetic modification of the cells and the risk of tumorigenesis. Another method is to produce from the reprogramming genes, recombinant proteins modified to permit their penetration of the plasma and nuclear membranes. Other reprogramming factors include, but are not limited to, small compounds synthesized through medicinal chemistry.

The method preferably further comprises isolating clonal lines of BiPS of the disclosure. For instance, the method preferably further comprises isolating clonal lines of BiPS of the disclosure by limiting dilution or the manual ‘picking’ of individual colonies.

Standard methods known in the art may be used to determine the detectable expression and level of expression of the various markers discussed above. Suitable methods include, but are not limited to, immunocytochemistry, flow cytometry, western blotting and quantitative PCR.

### III. Viruses and Viral Sequences

Provided herein are also methods and compositions for using the viruses and viral sequences identified herein from the bat pluripotent stem cells. In particular, viruses, viral families, and viral sequences are disclosed herein.

In some embodiments, the method of obtaining viral sequences from bat IPSCs, comprises obtaining bat IPSCs; identifying viral sequences residing in the bat iPSC genome or intracellular virus genome; and assembling the viral sequences. In some embodiments, the bat IPSCs (BiPS) are produced by the methods described above. In some embodiments, the nucleic acid sequences are obtained by sequencing RNA transcripts such as RNA seq, long read sequencing such ss Iso-seq (PacBio), or sequencing the genomic DNA such as by DNA sequencing of samples derived from the BiPS. In some embodiments, amino acid sequences can be obtained by LC-MS or amino acid sequencing of samples derived from the BiPS. In some embodiments the samples can be derived directly from the BiPS or the medium BiPS were grown in. In some embodiments, the samples can be derived from differentiated cells derived from the BiPS.

In some embodiments, the obtained nucleic acid sequences are assembled into longer nucleic acid sequences. Short and long assembled sequences can be classified as potentially viral origin or non-viral origin for example as described in Example 10. The sequences can be further classified into virus clades by comparing with known sequences from virus nucleic acids in databases such as the NCBI Assembly database (www.ncbi.nlm.nih.gov/assembly) or Virus Pathogen Resource (www.viprbrc.org/brc/home.spg?decorator=vipr). Nucleic acid sequences can be also classified using metagenomic classifiers, such as Kraken2.

TABLE 1 Exemplary virus families and viruses found in a taxonomic distribution of virome reads from BiPS as determined by the metagenomic classifier Kraken2.

TABLE 1

| Virus Family | Virus |
|---|---|
| Retroviridae | ND |
| Picornavirales | Rotavirus |
| Coronaviridae | ND |
| Hantaviridae | ND |
| Herpesvirales | ND |
| Poxviridae | ND |
| Adenoviridae | ND |
| Papillomaviridae | ND |
| Myoviridae | ND |
| Flaviviridae | ND |
| Siphoviridae | ND |
| Baculoviridae | ND |
| Duplondaviria | ND |
| Riboviria | ND |
| Filoviridae | Ebola |
| Filoviridae | Cueva |
| Filoviridae | Dianlovirus |
| Mononegavirales | ND |

ND, virus was not determined

More exemplary viral families, viruses and sequences identified from the BiPS are shown in TABLE A.

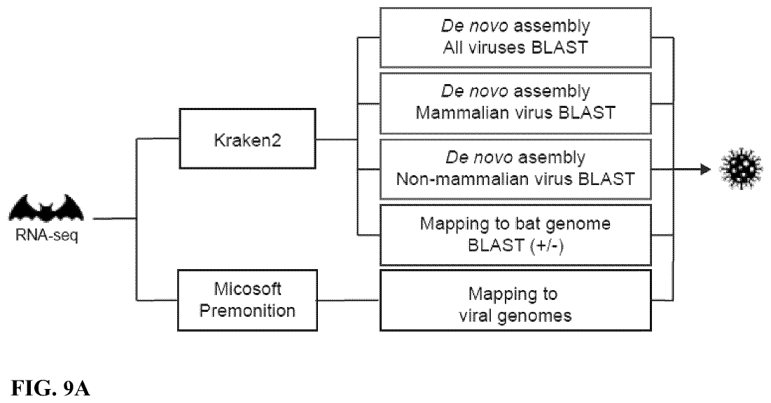

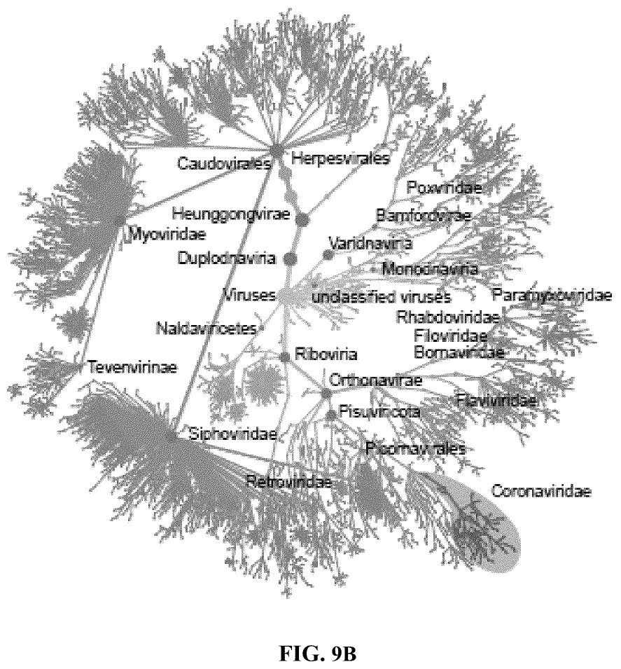

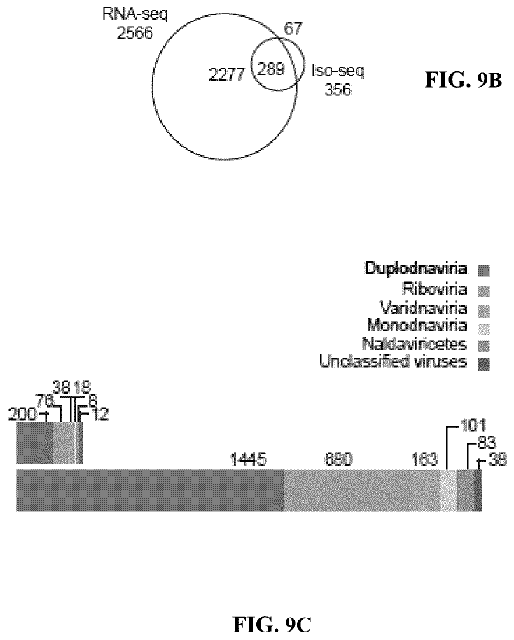

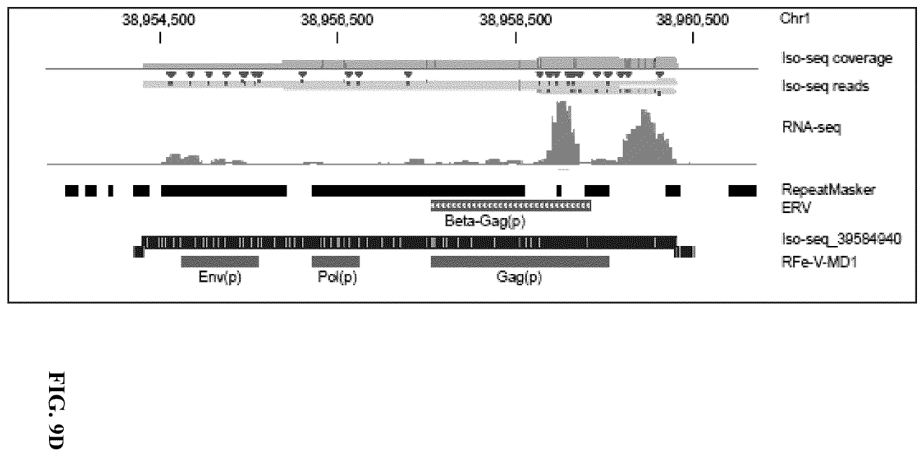

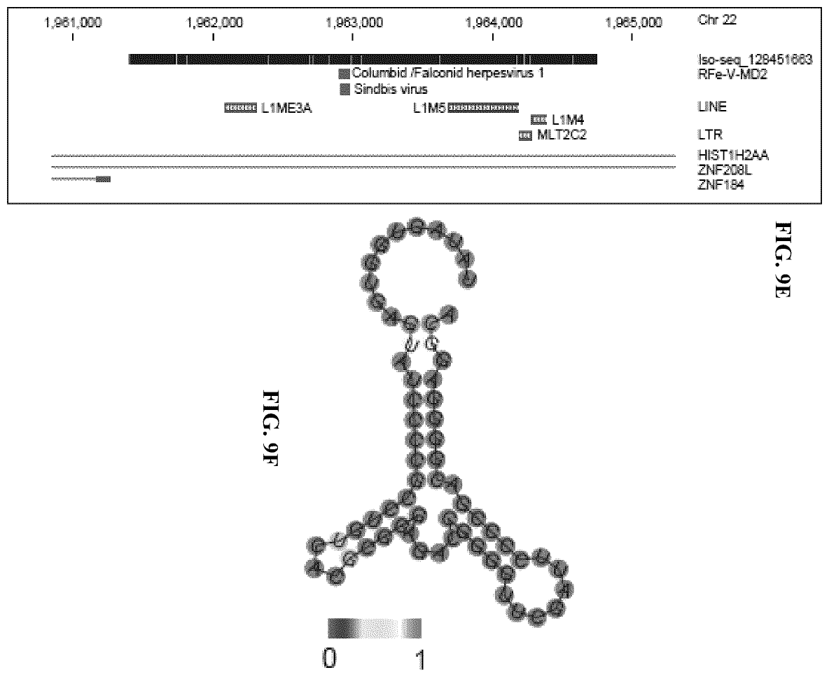

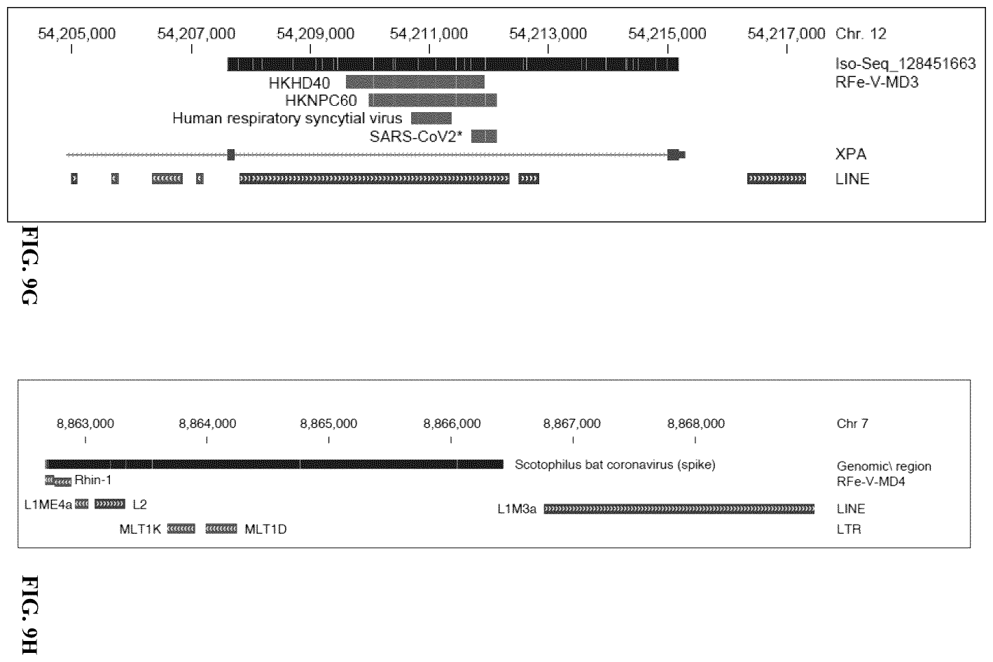

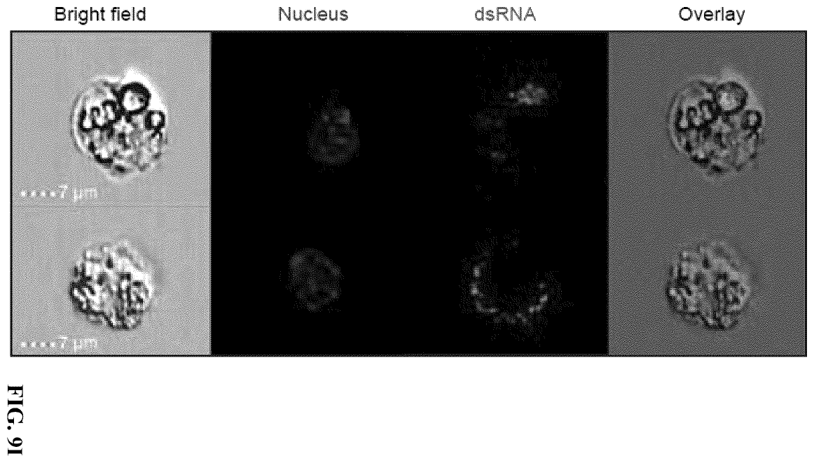

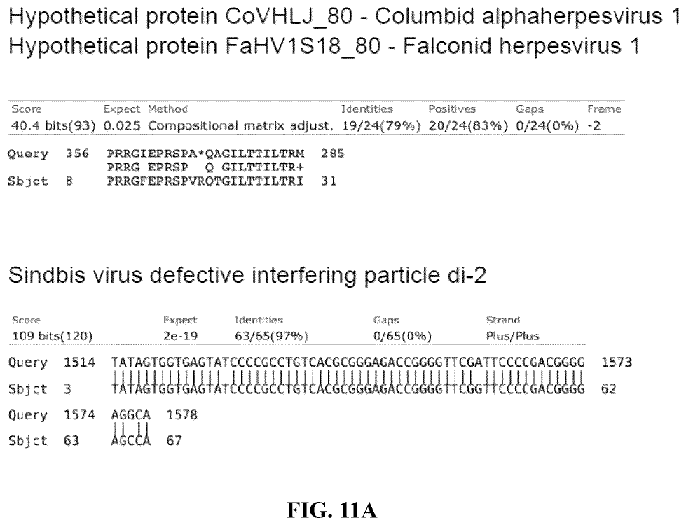

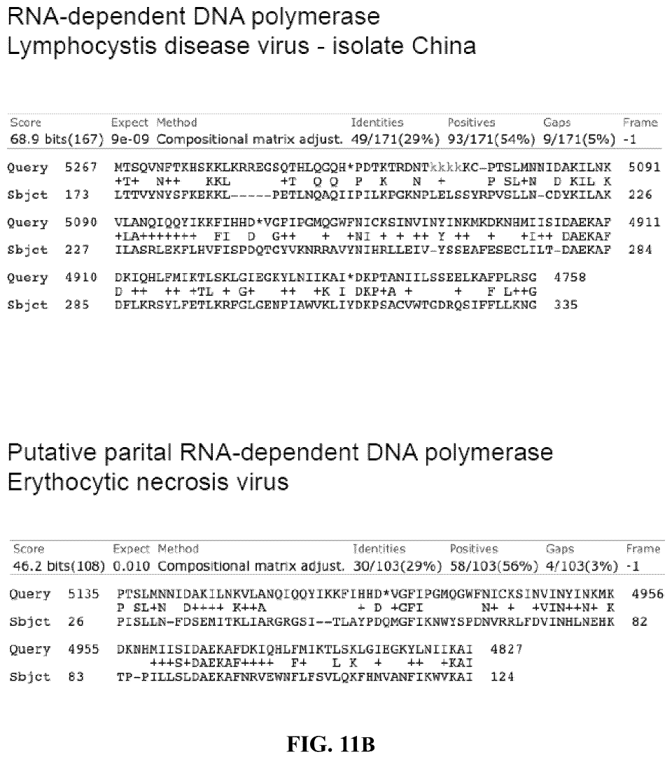

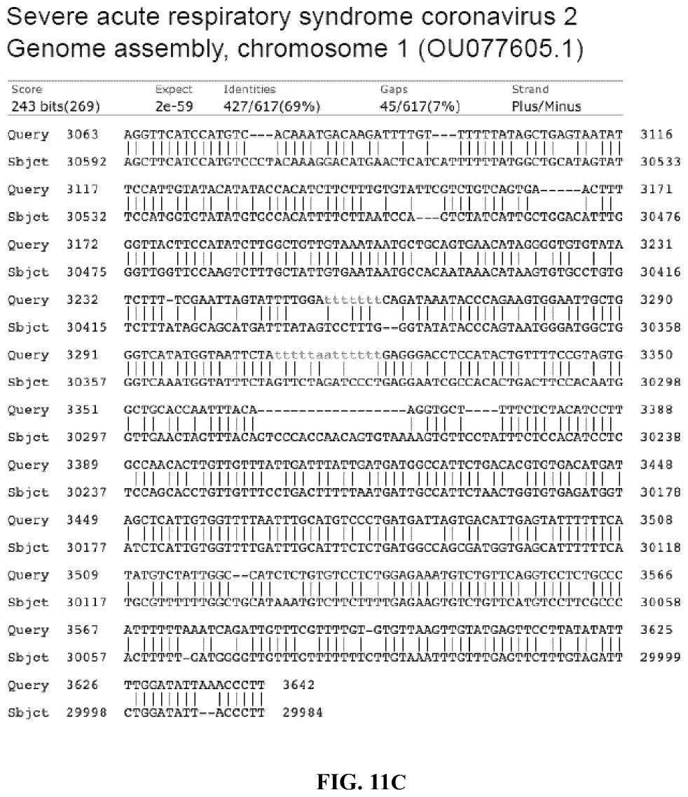

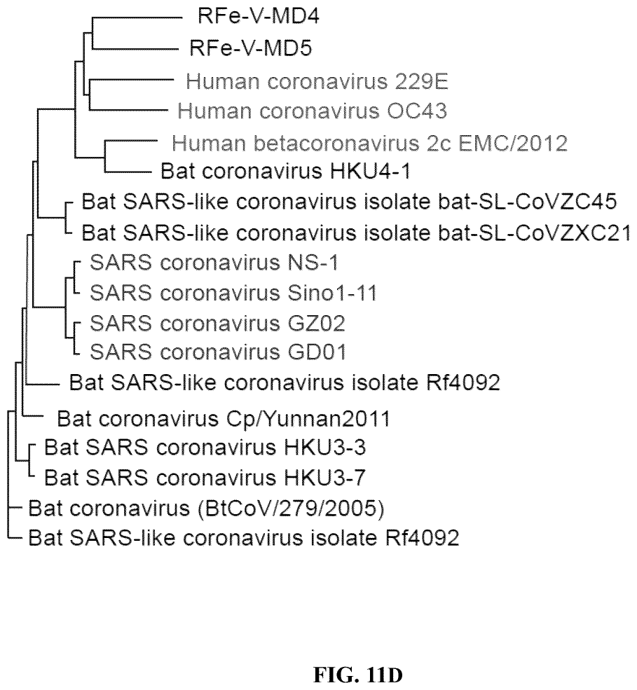

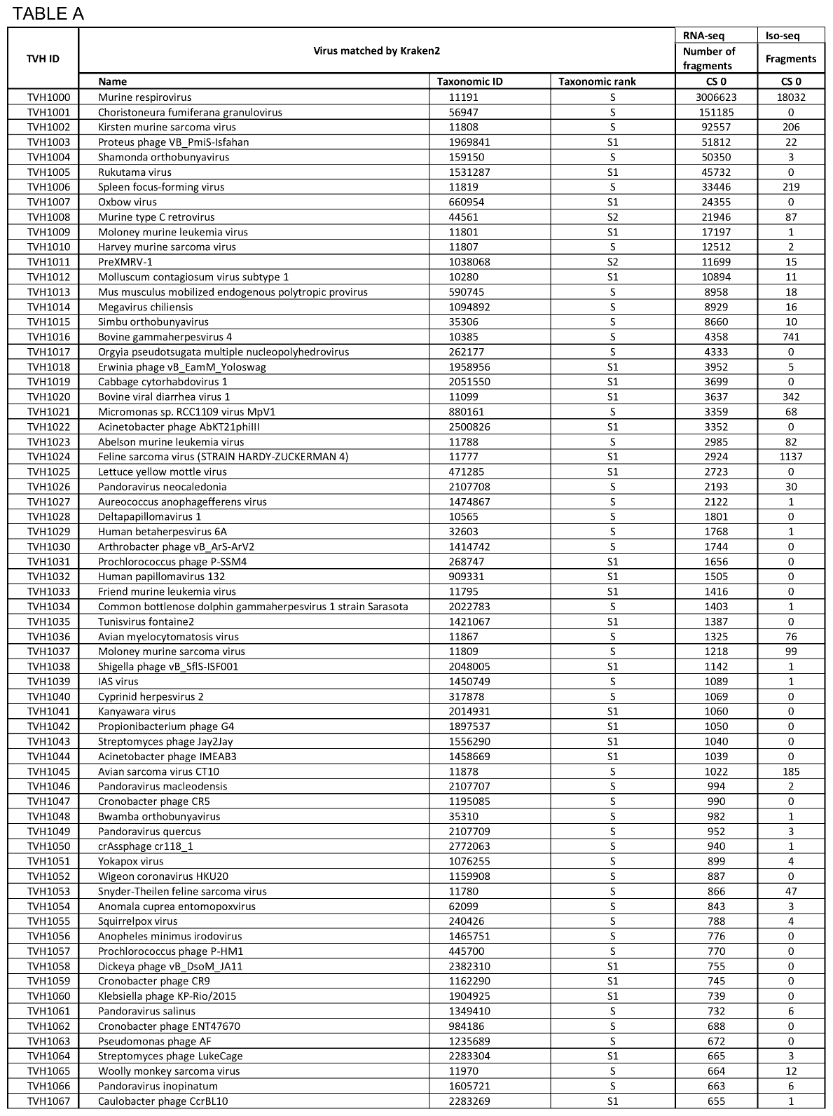

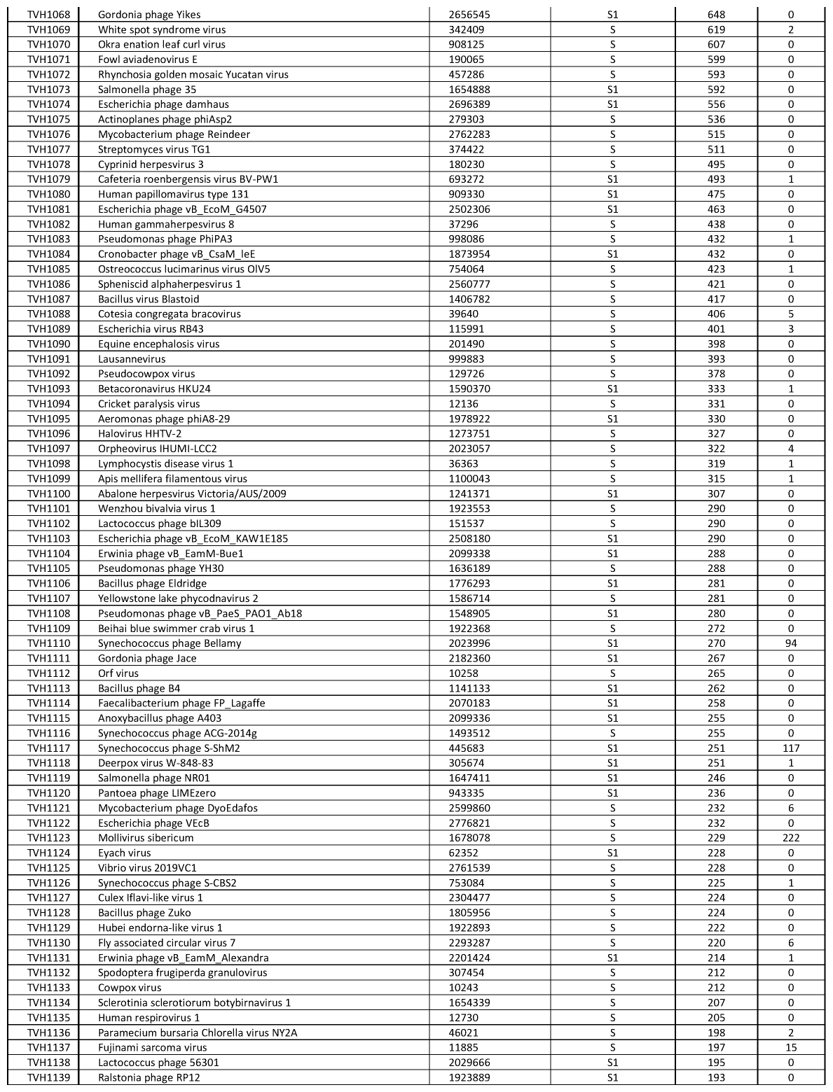

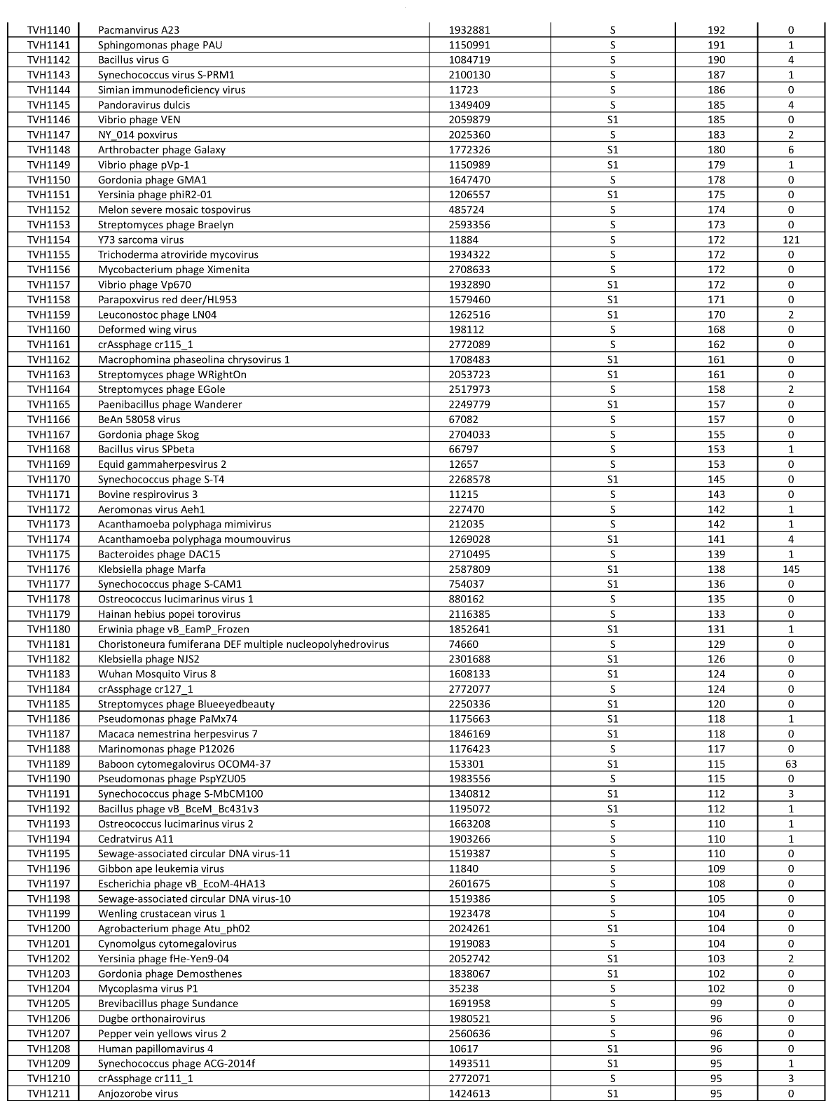

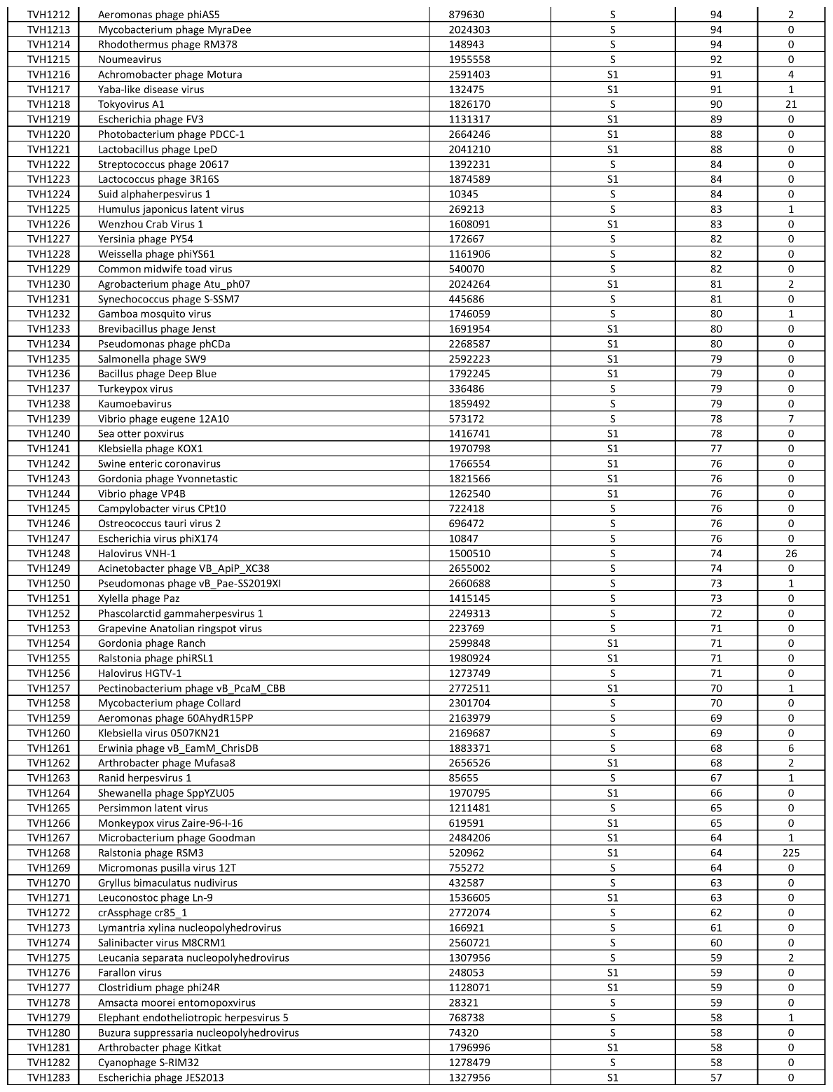

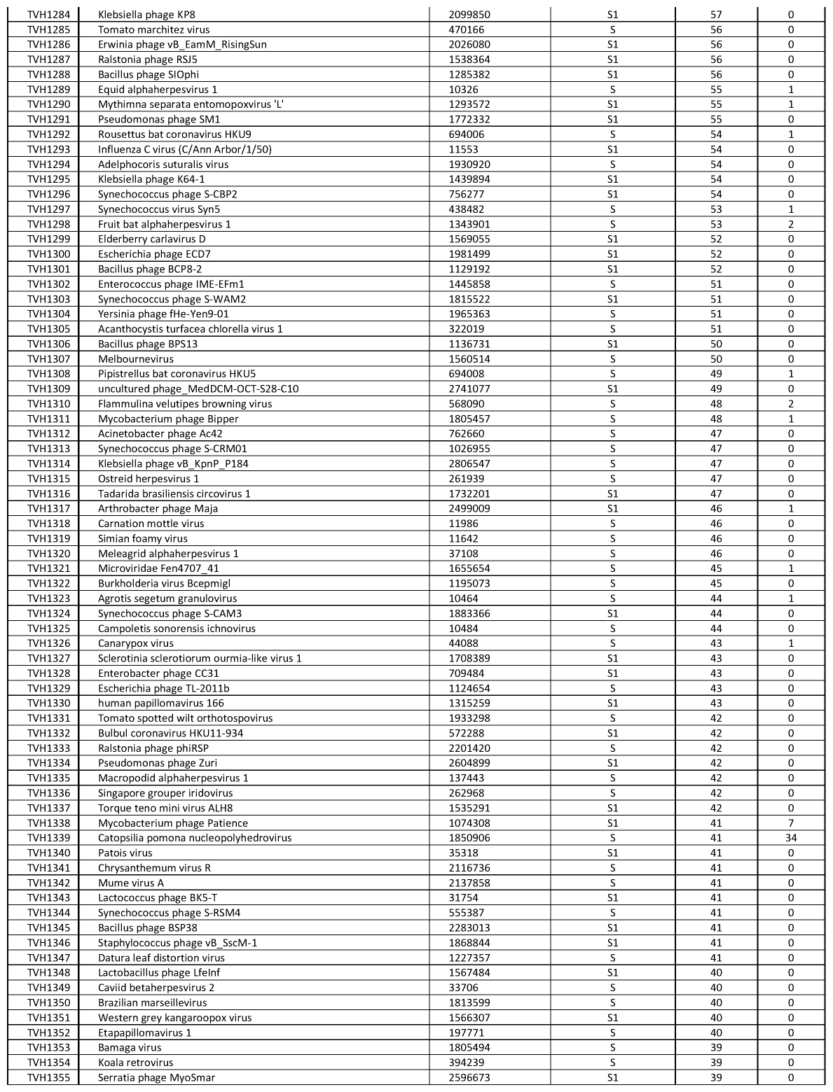

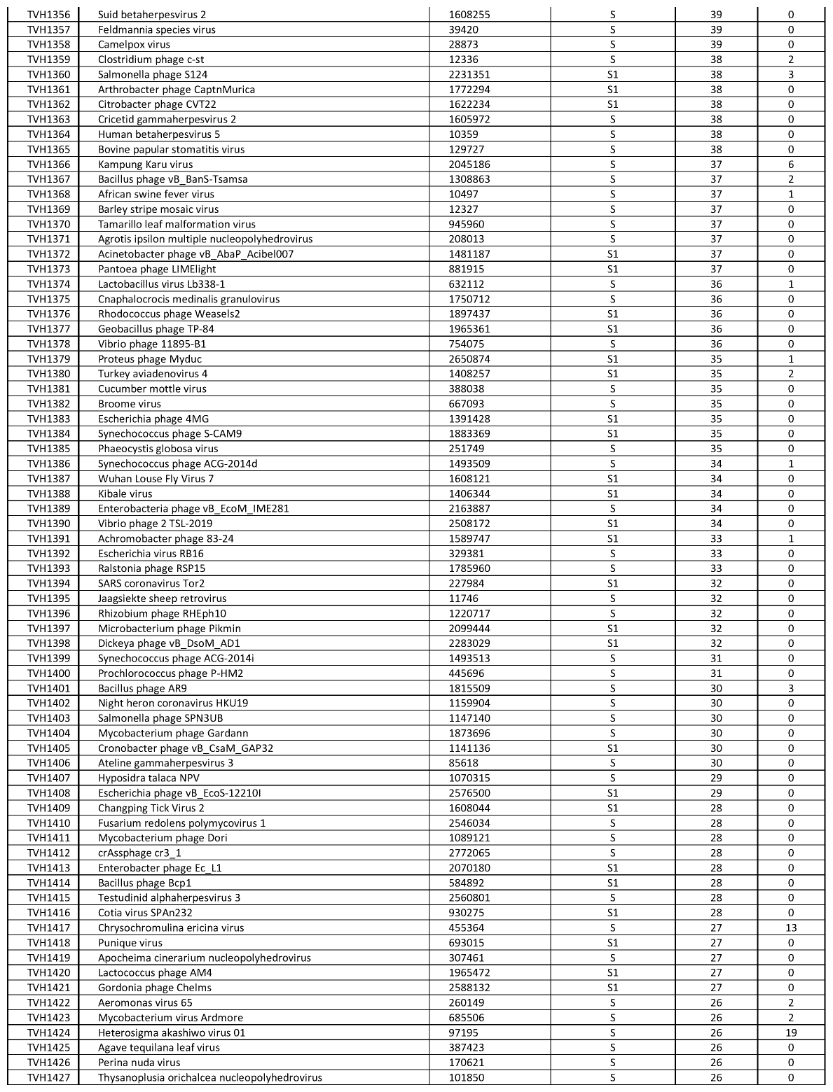

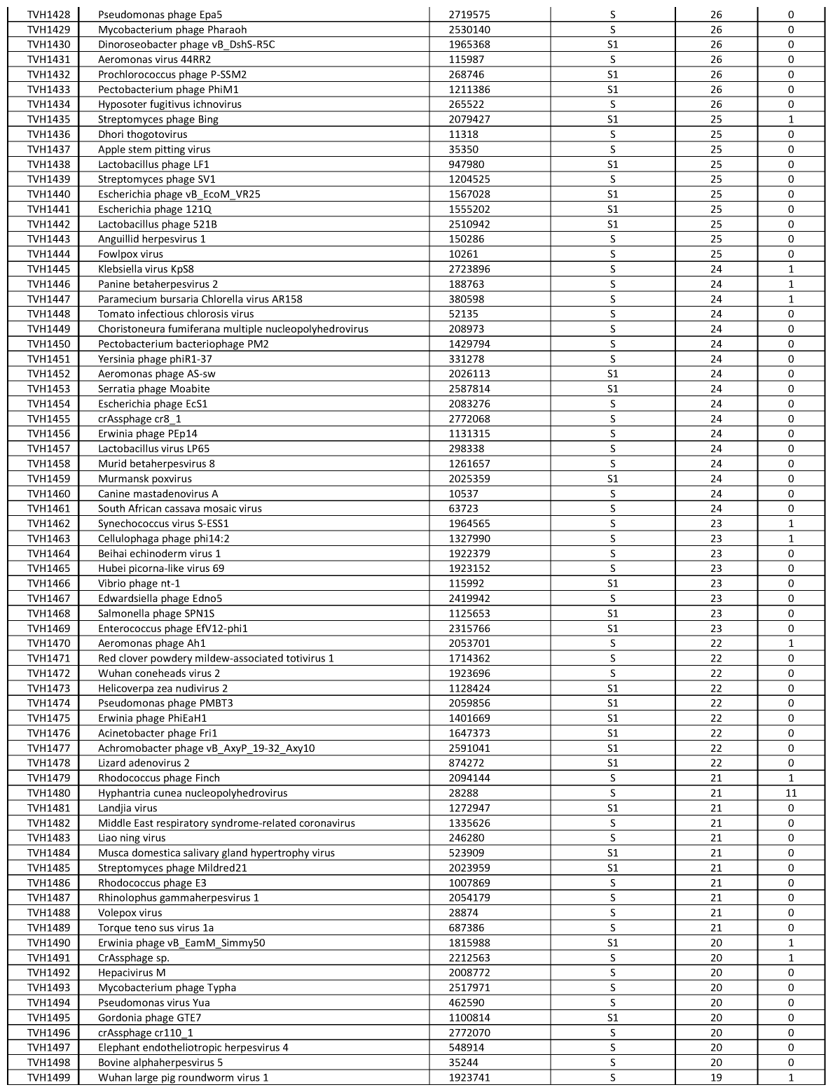

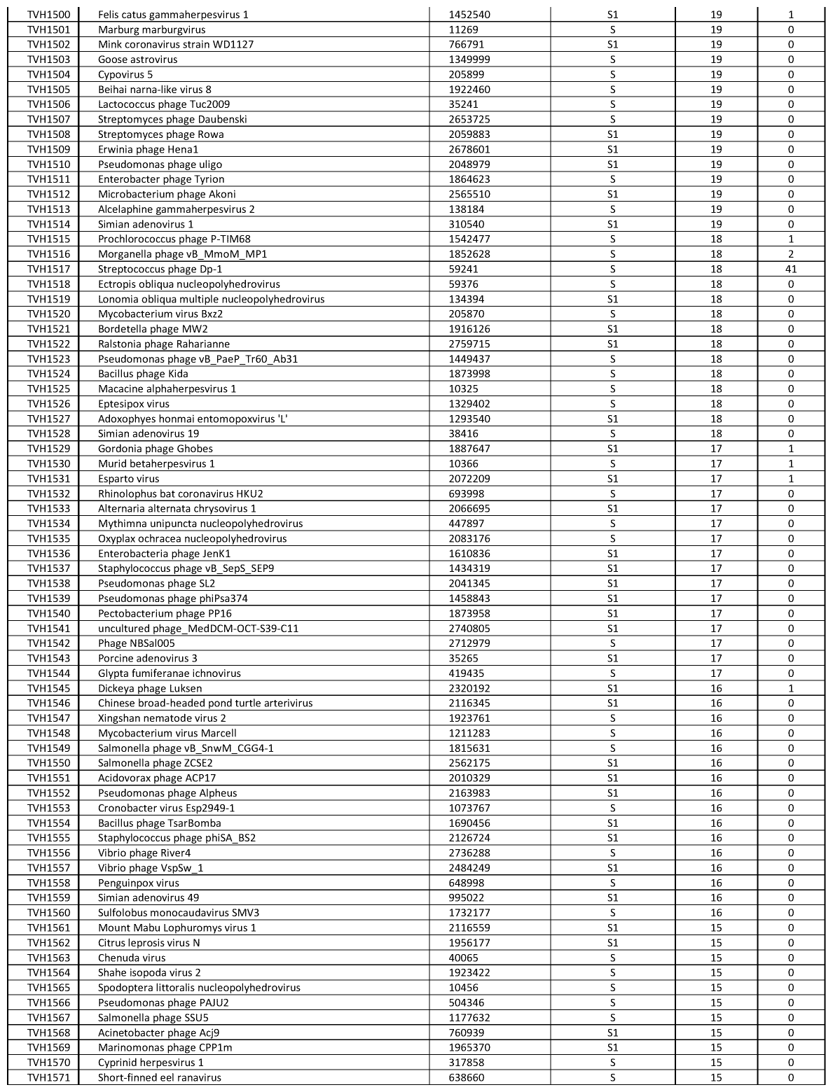

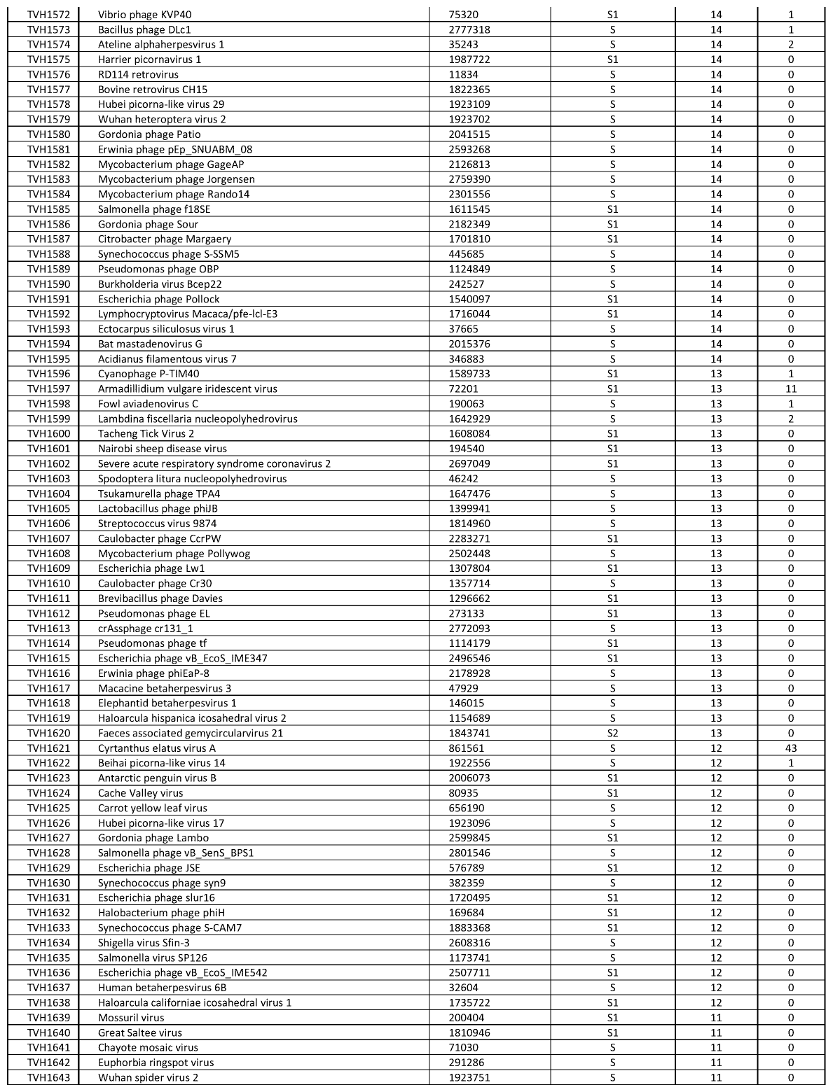

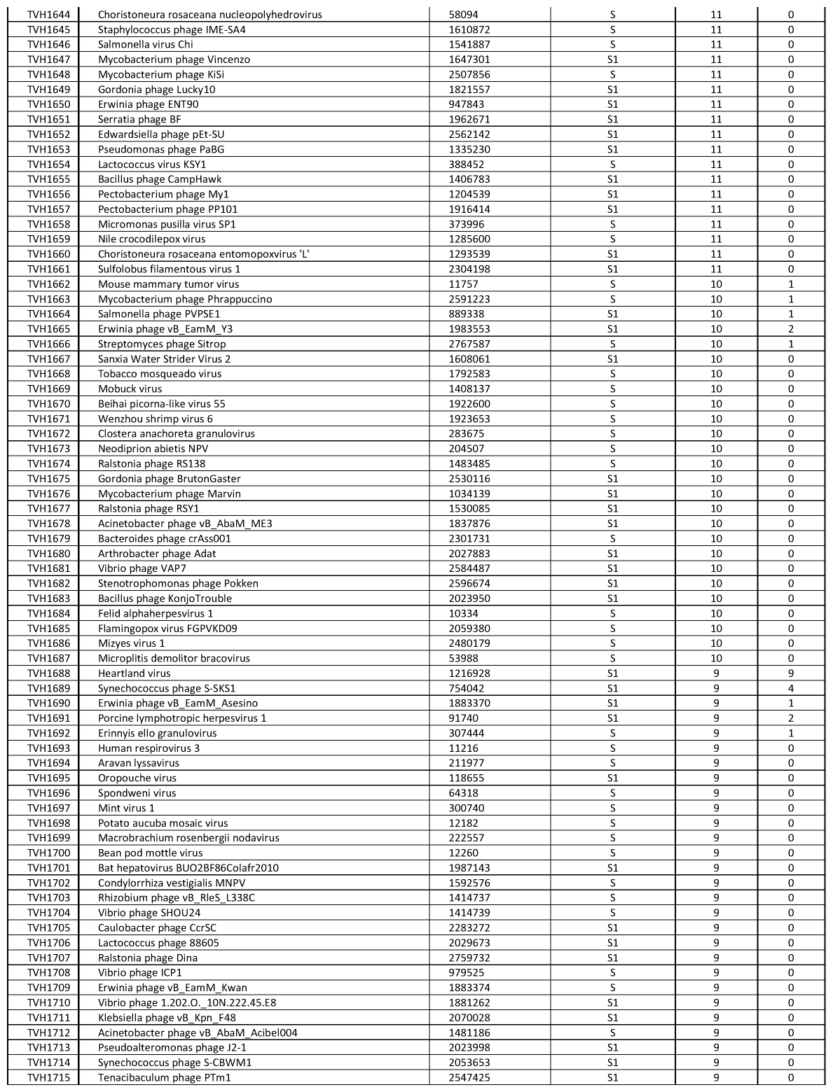

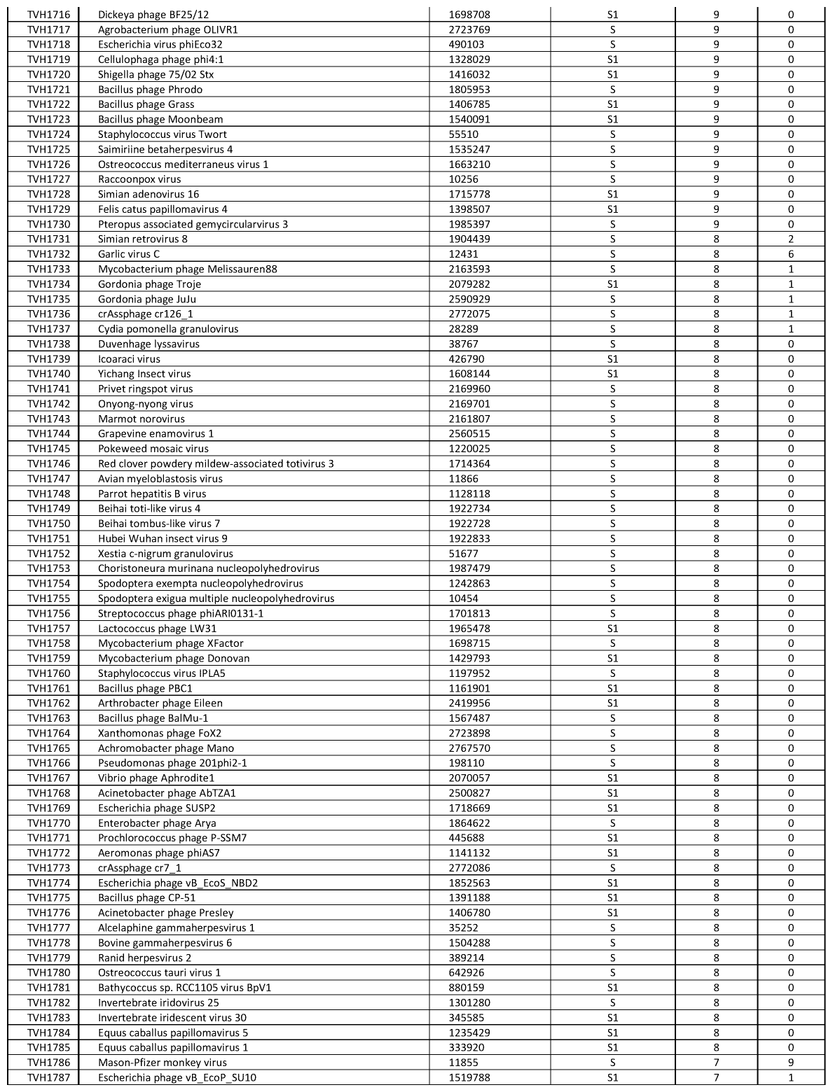

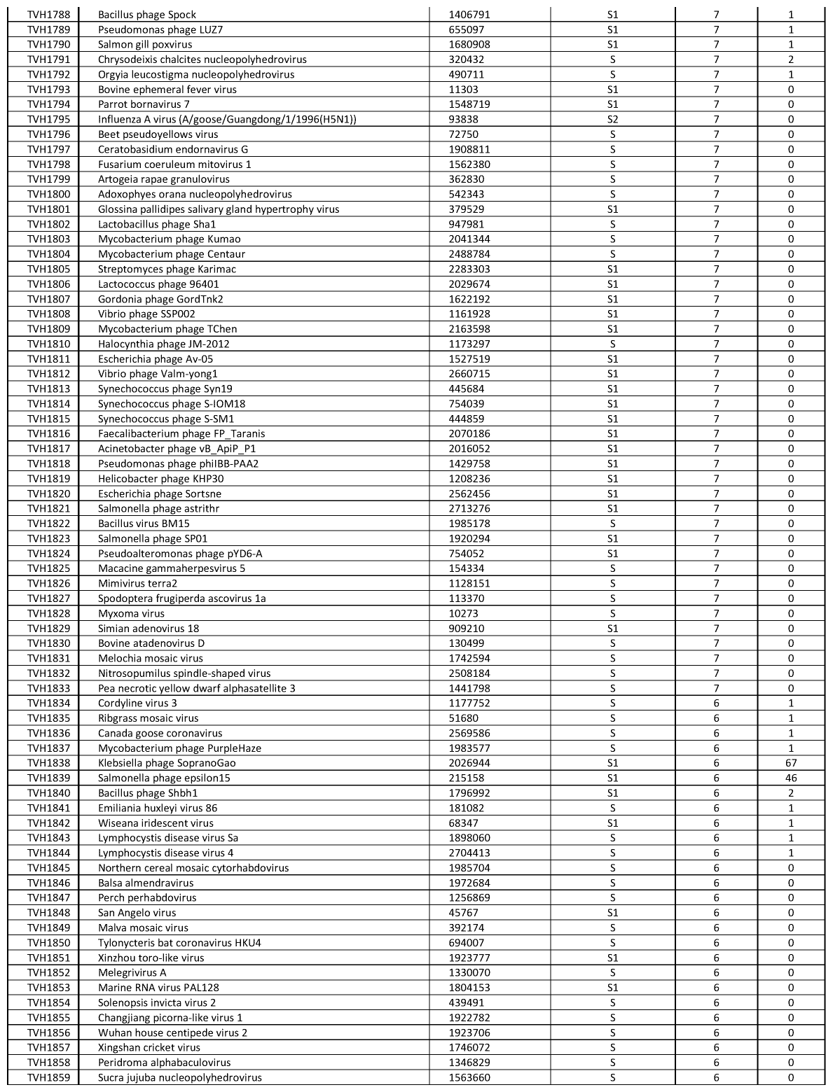

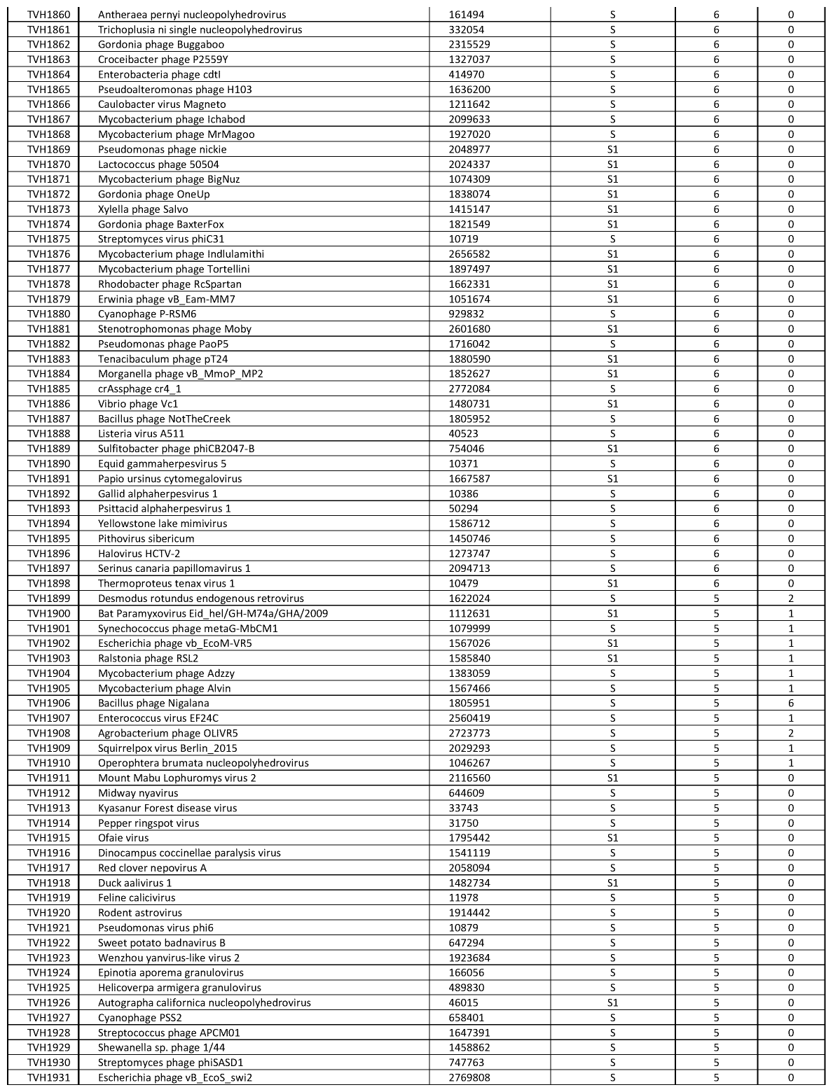

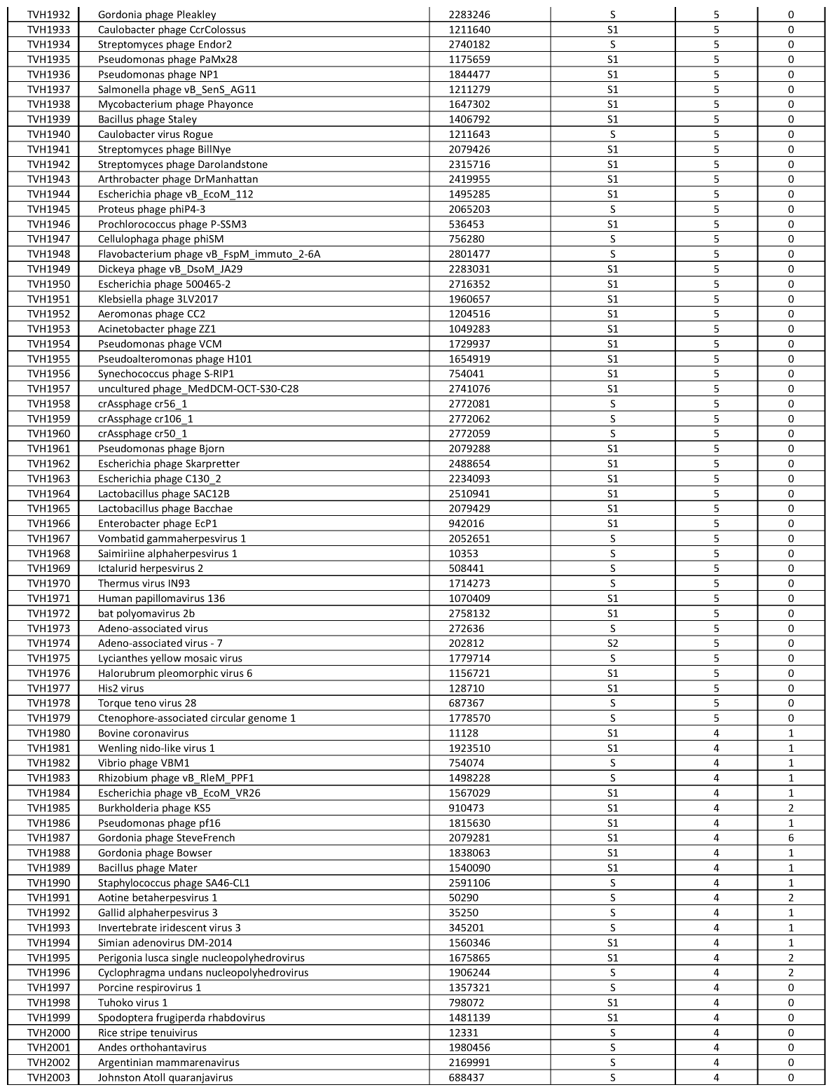

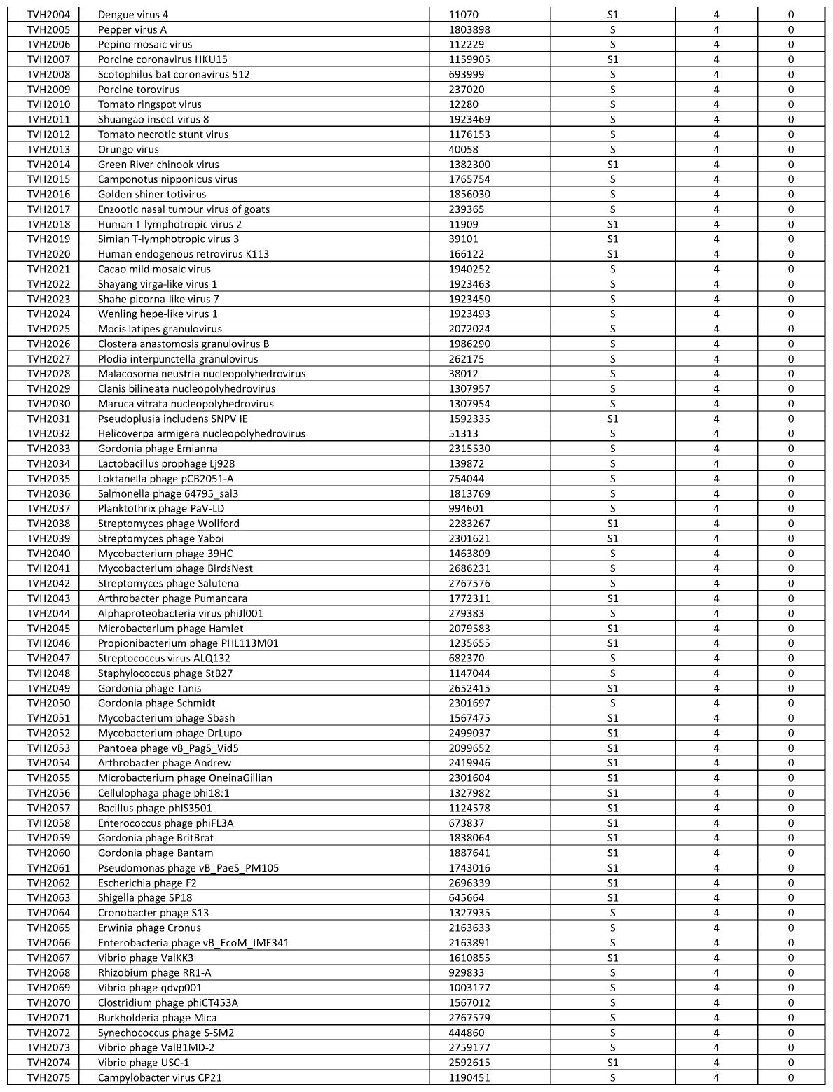

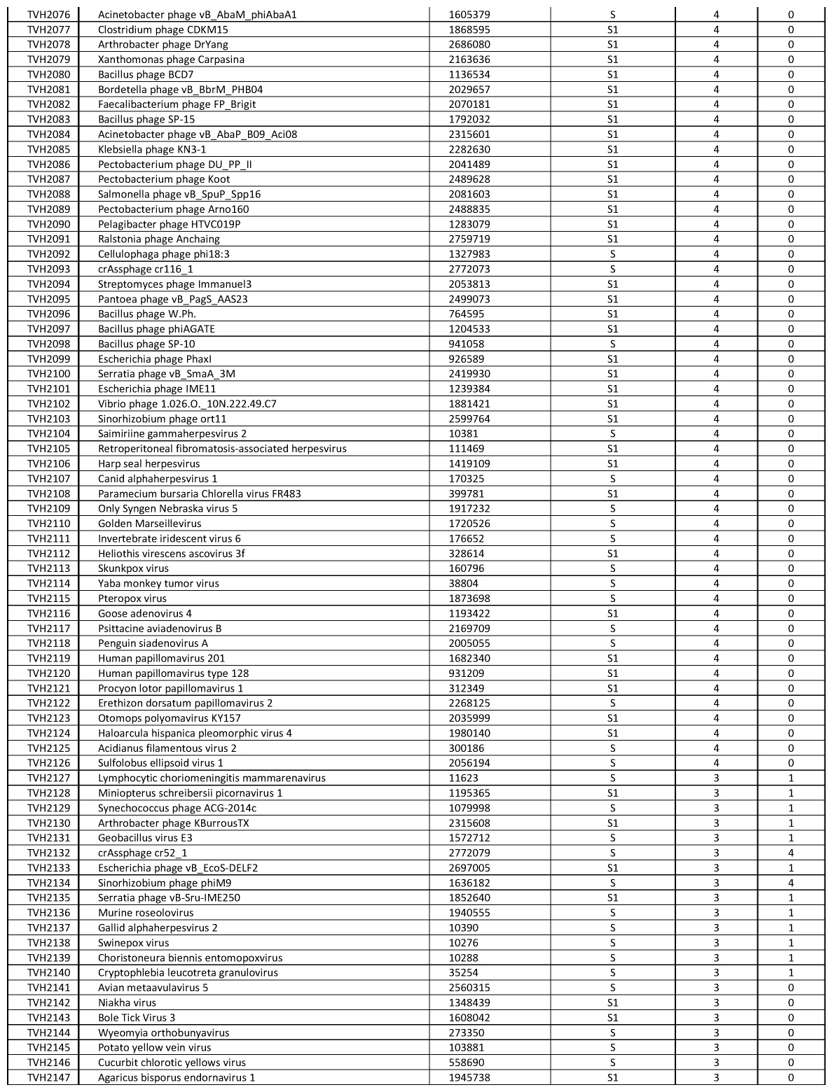

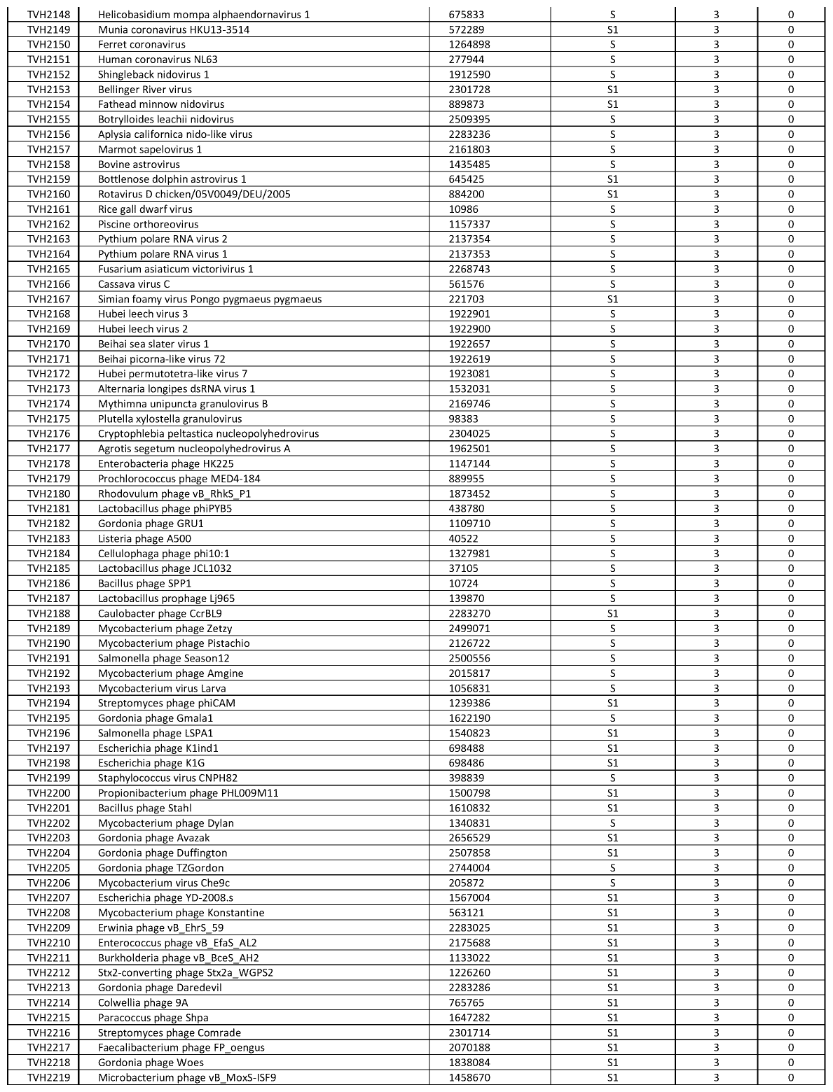

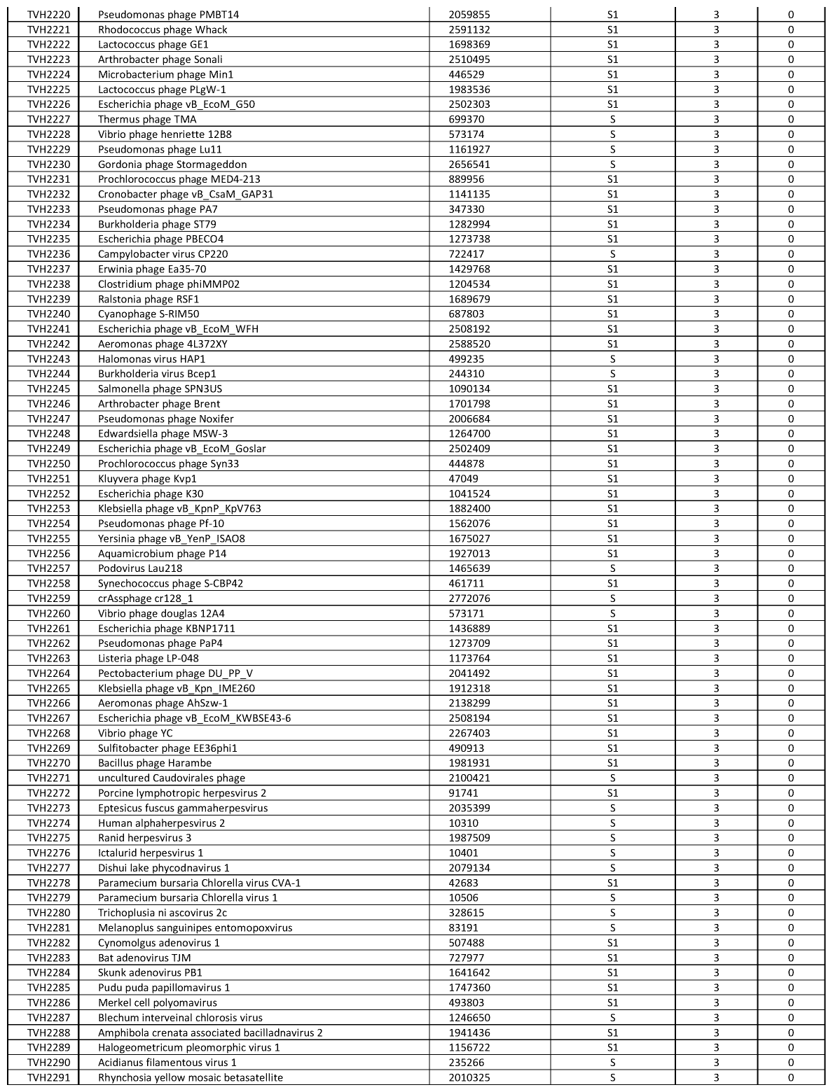

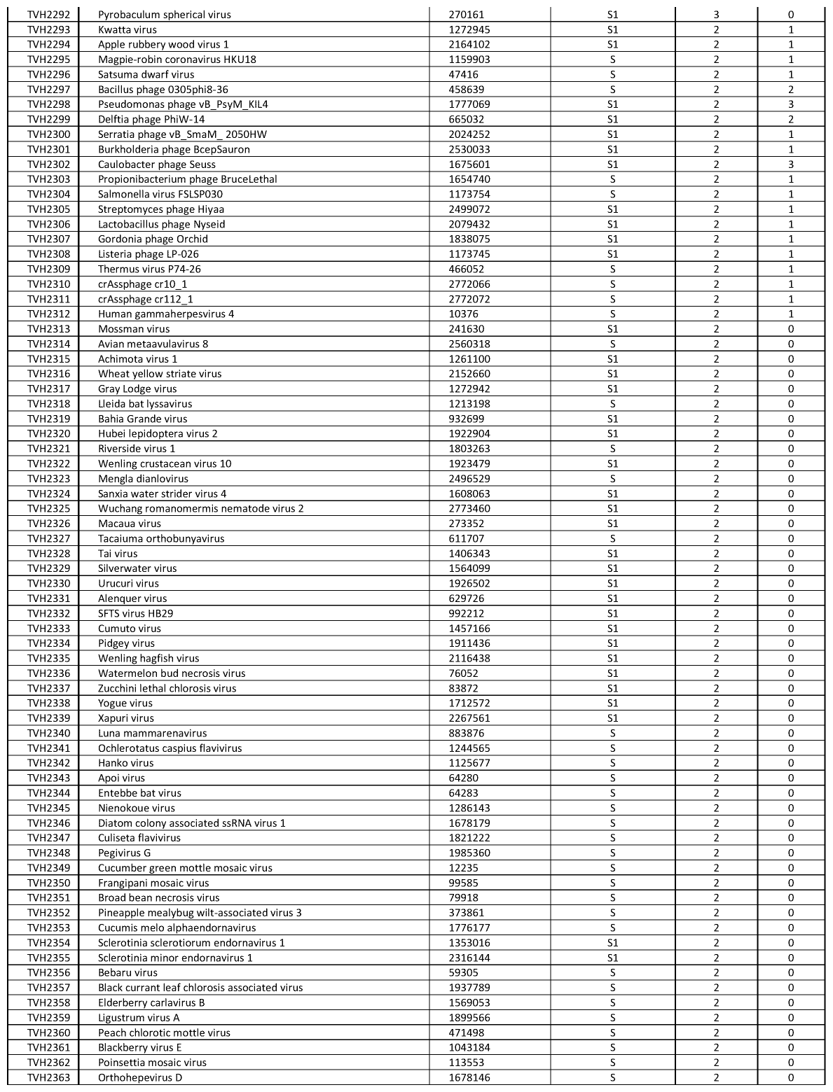

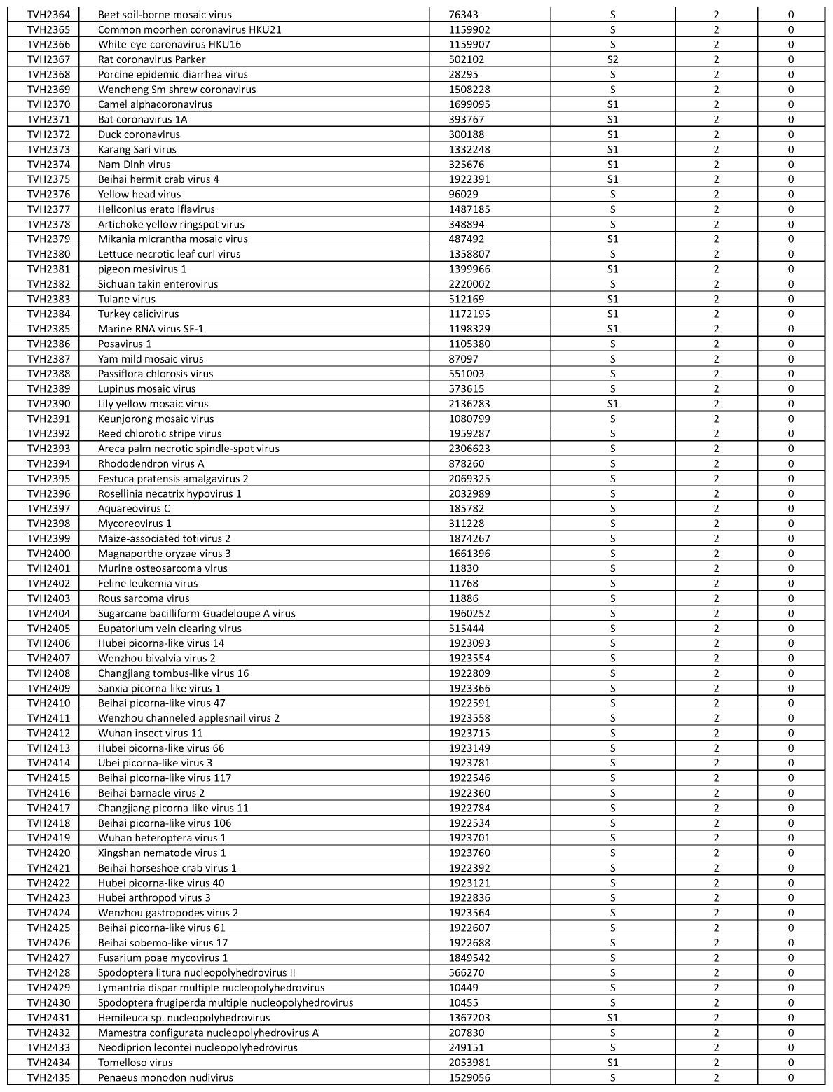

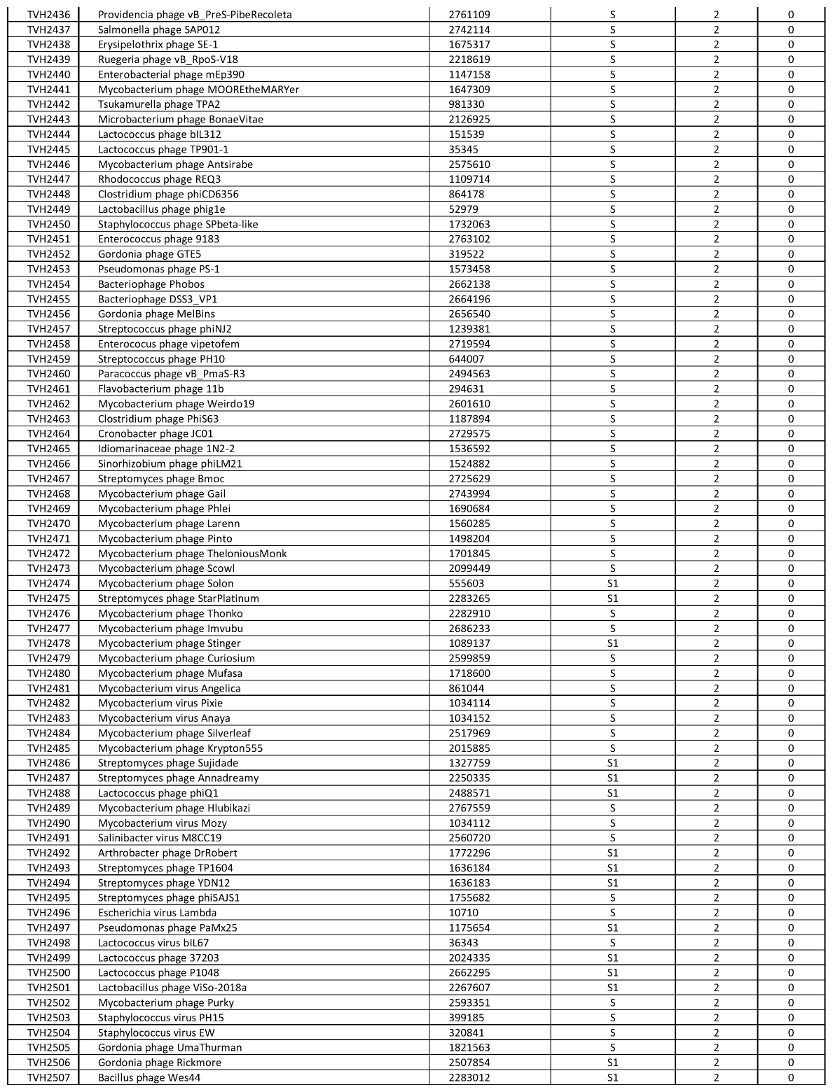

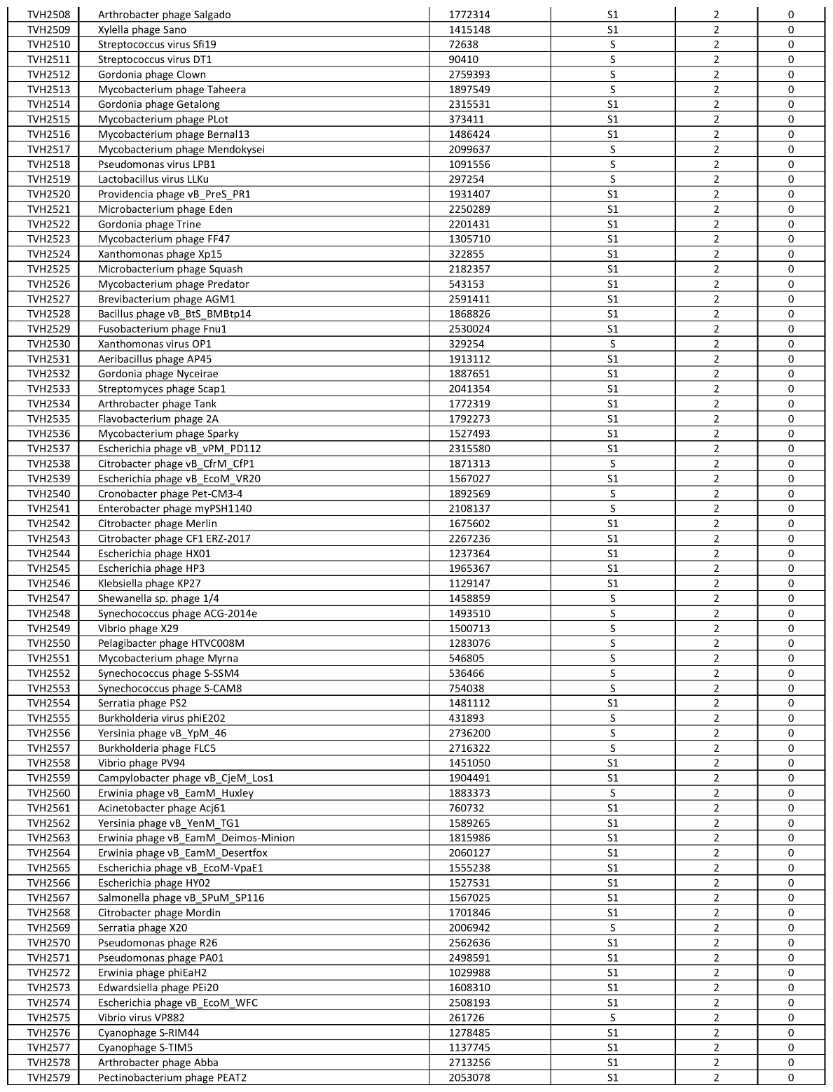

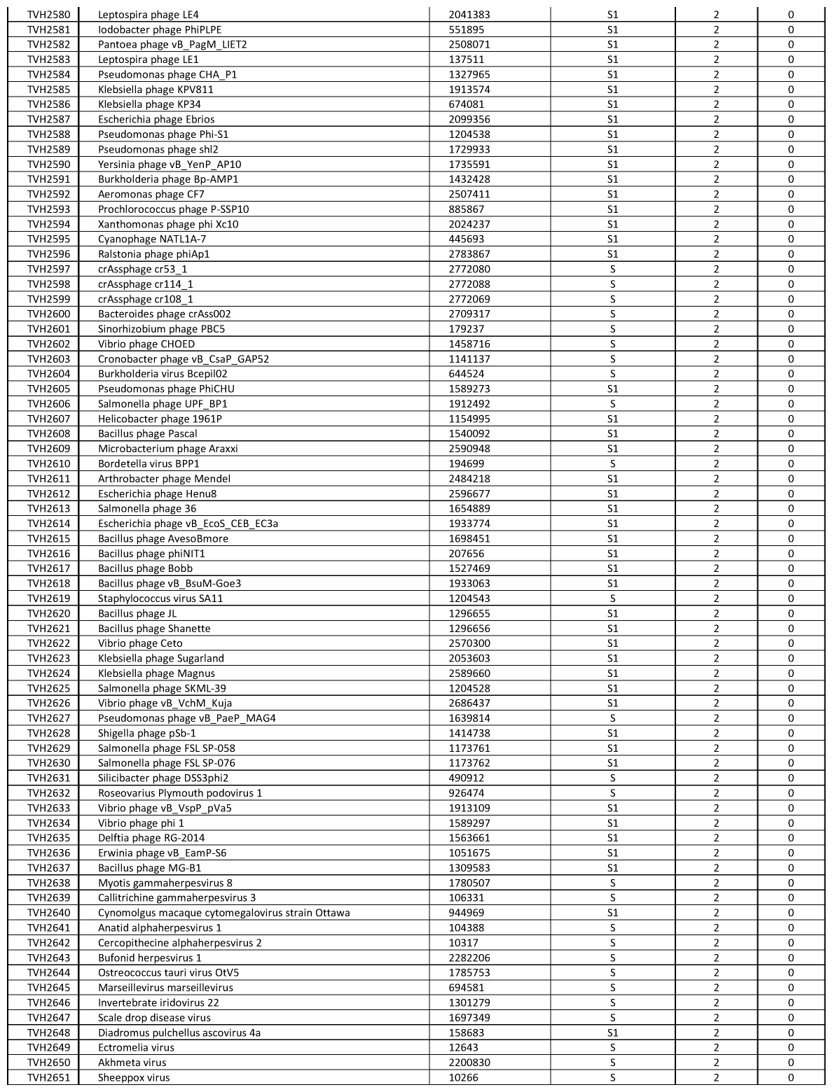

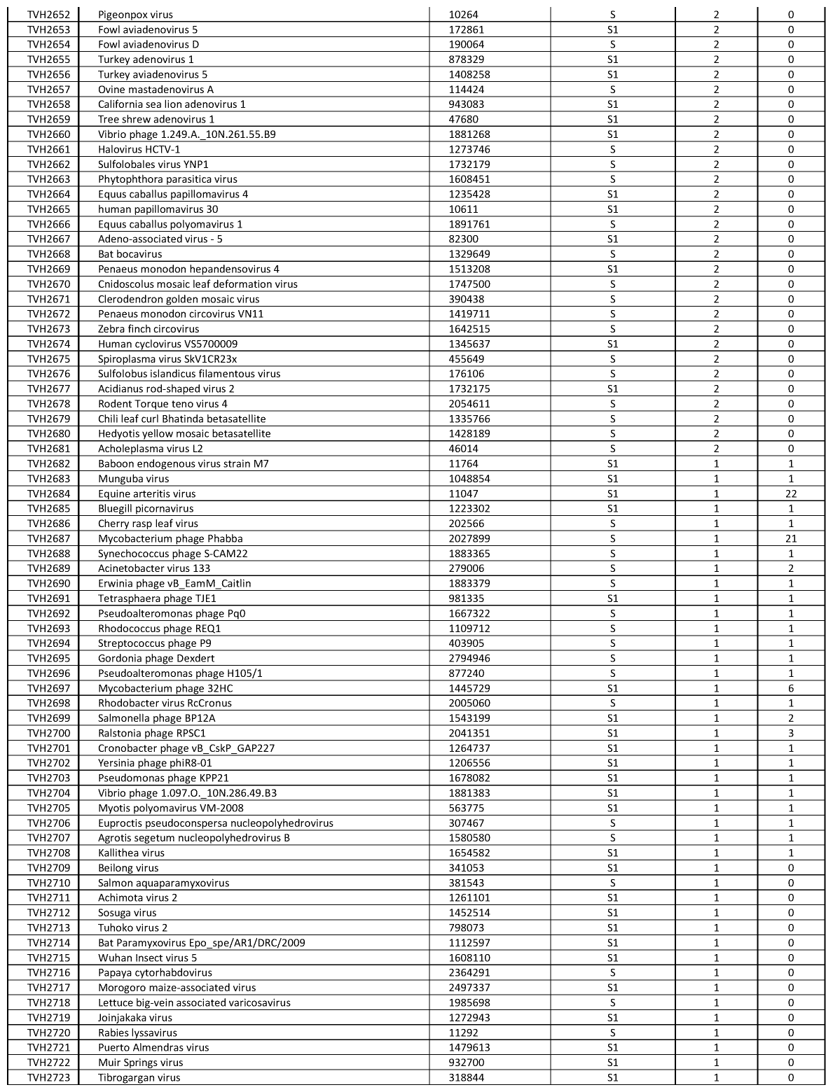

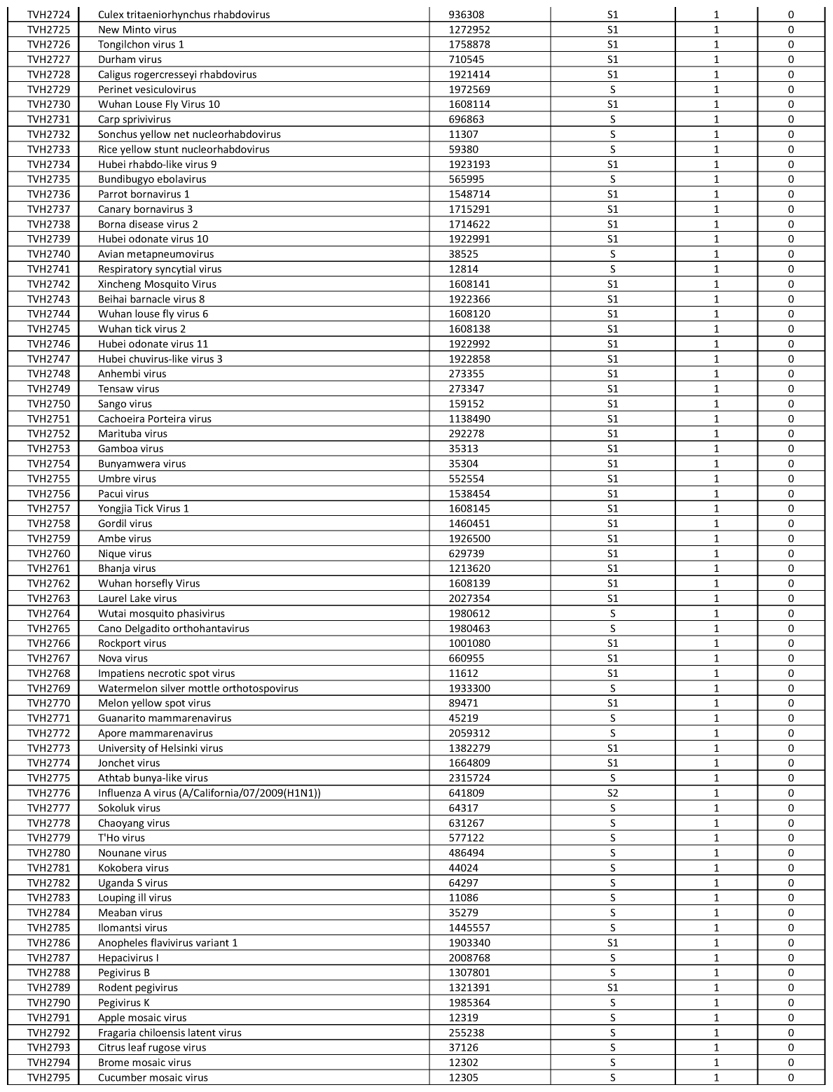

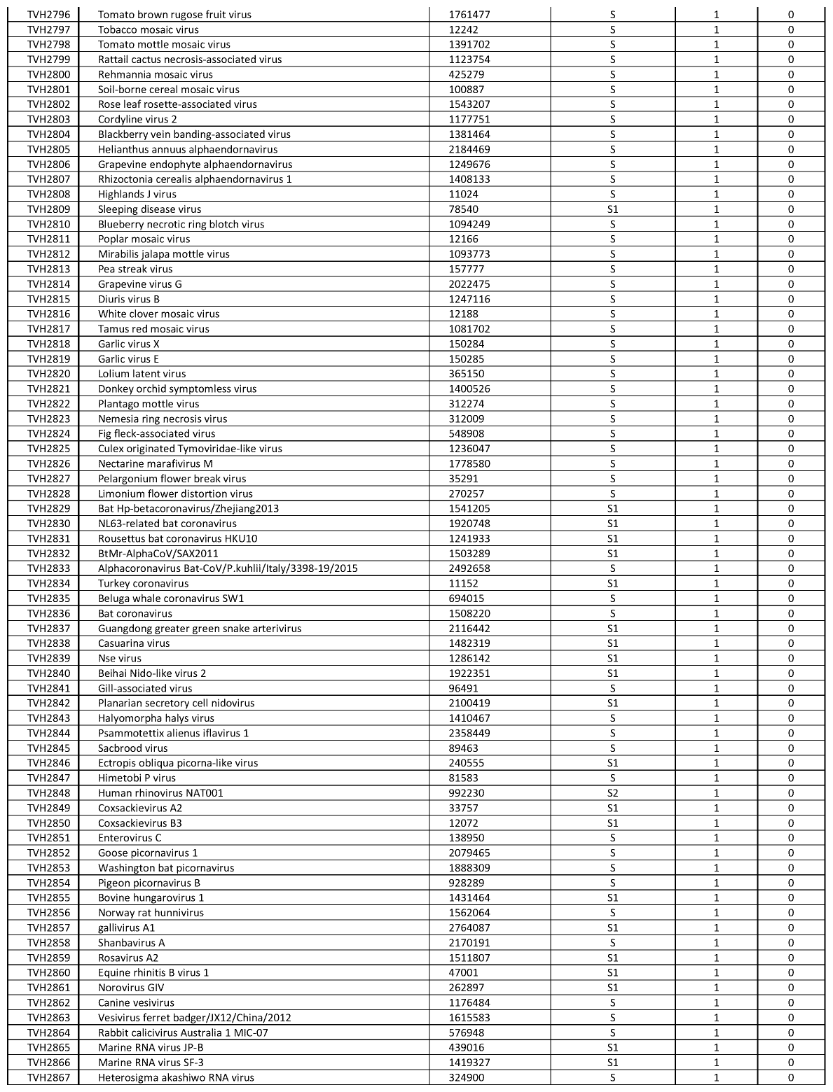

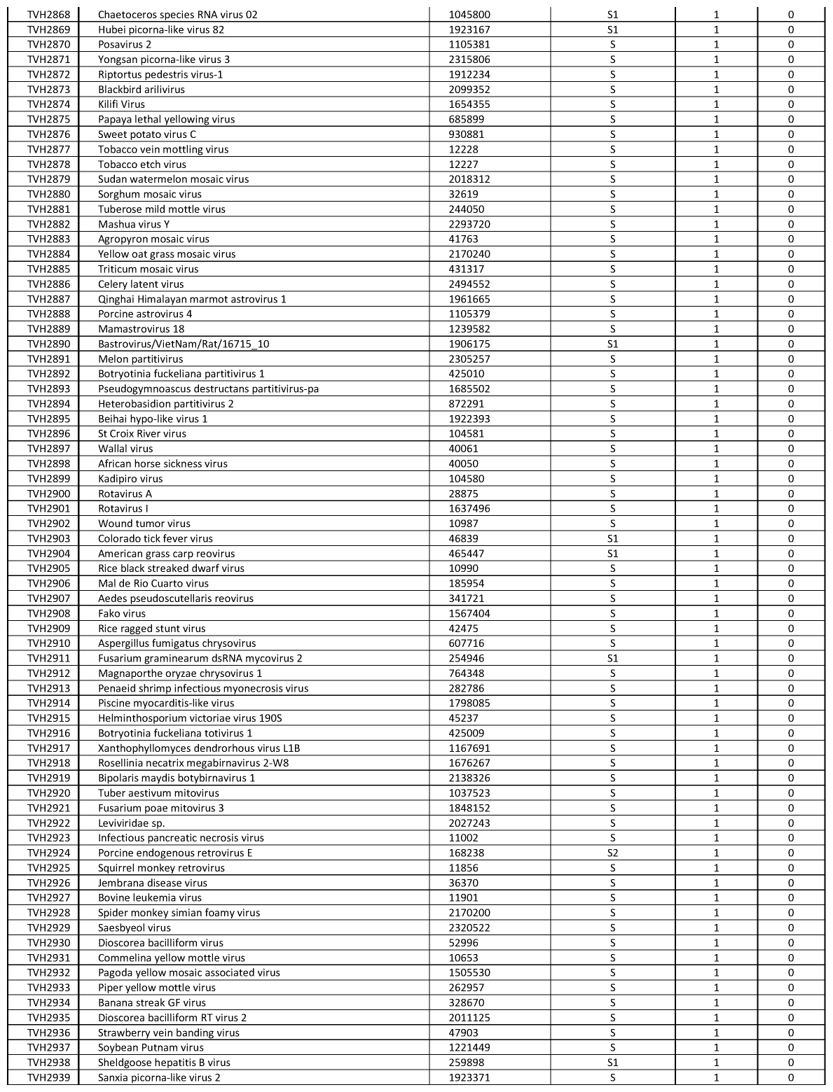

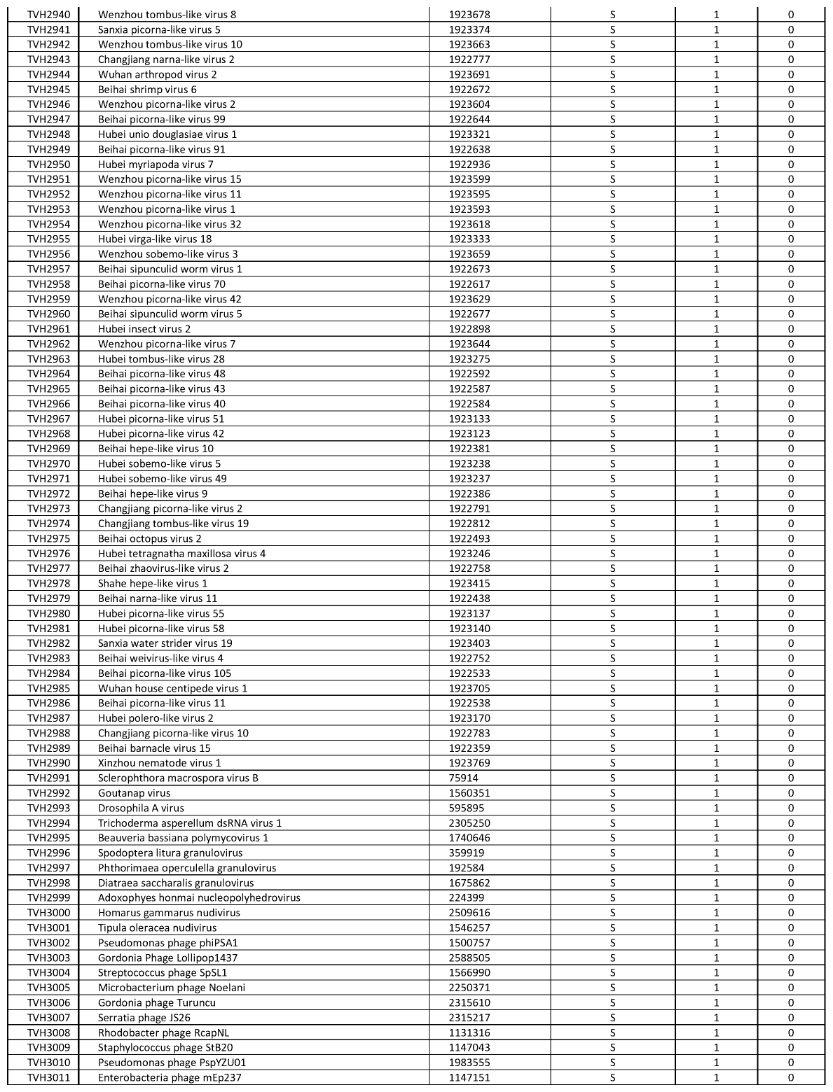

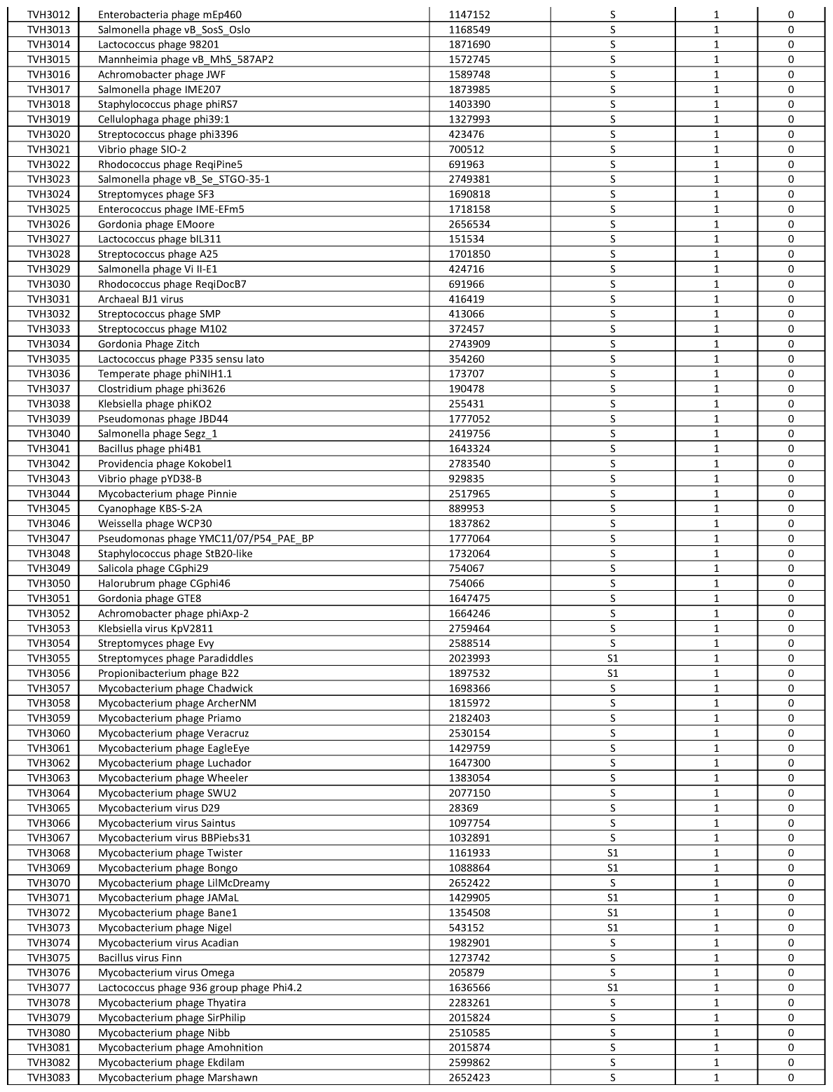

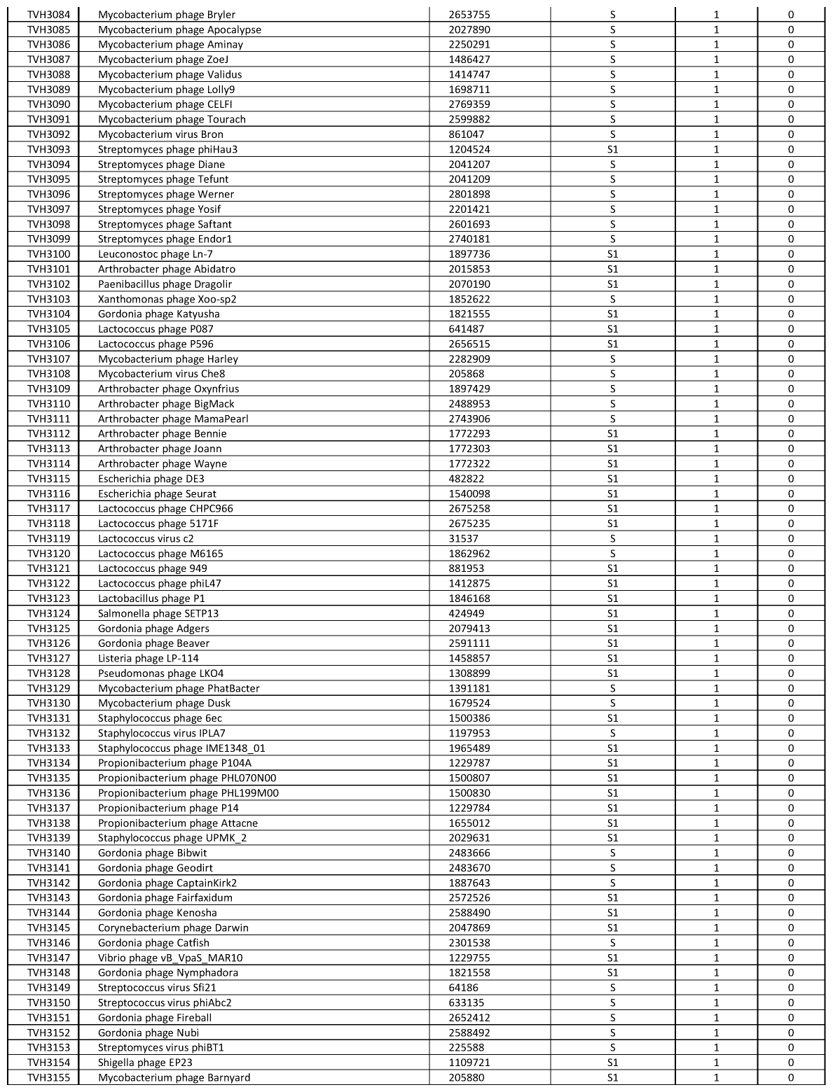

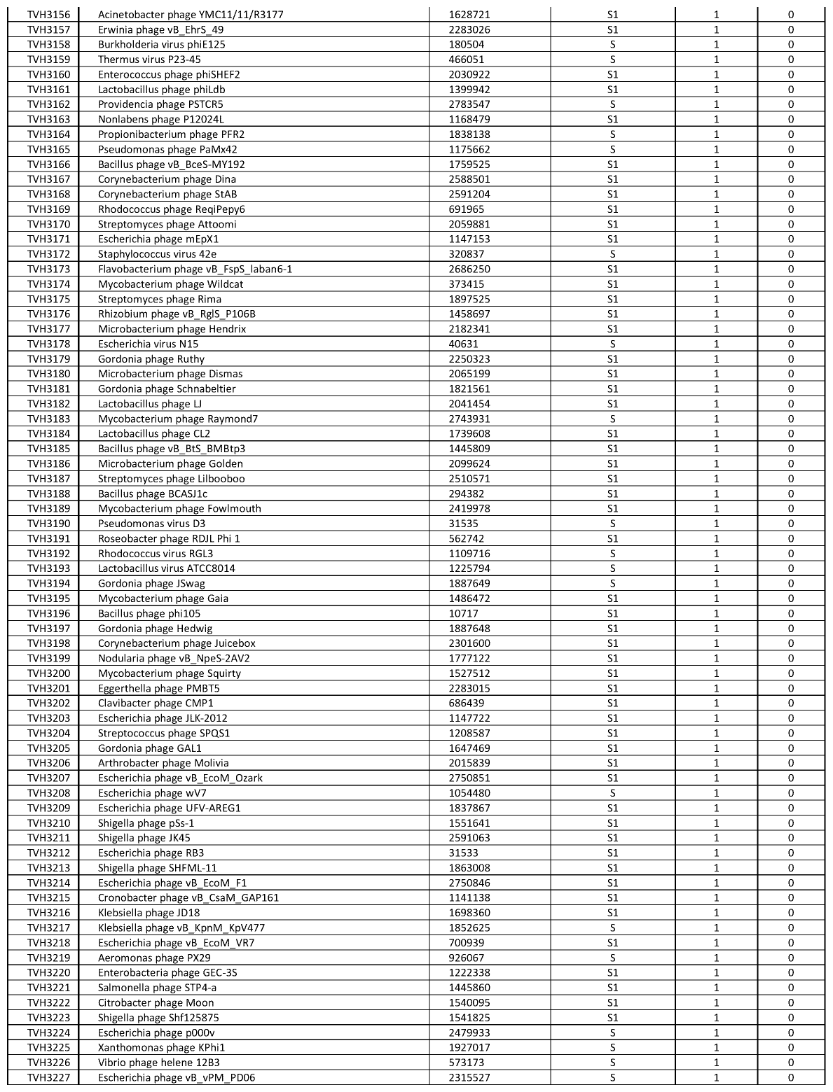

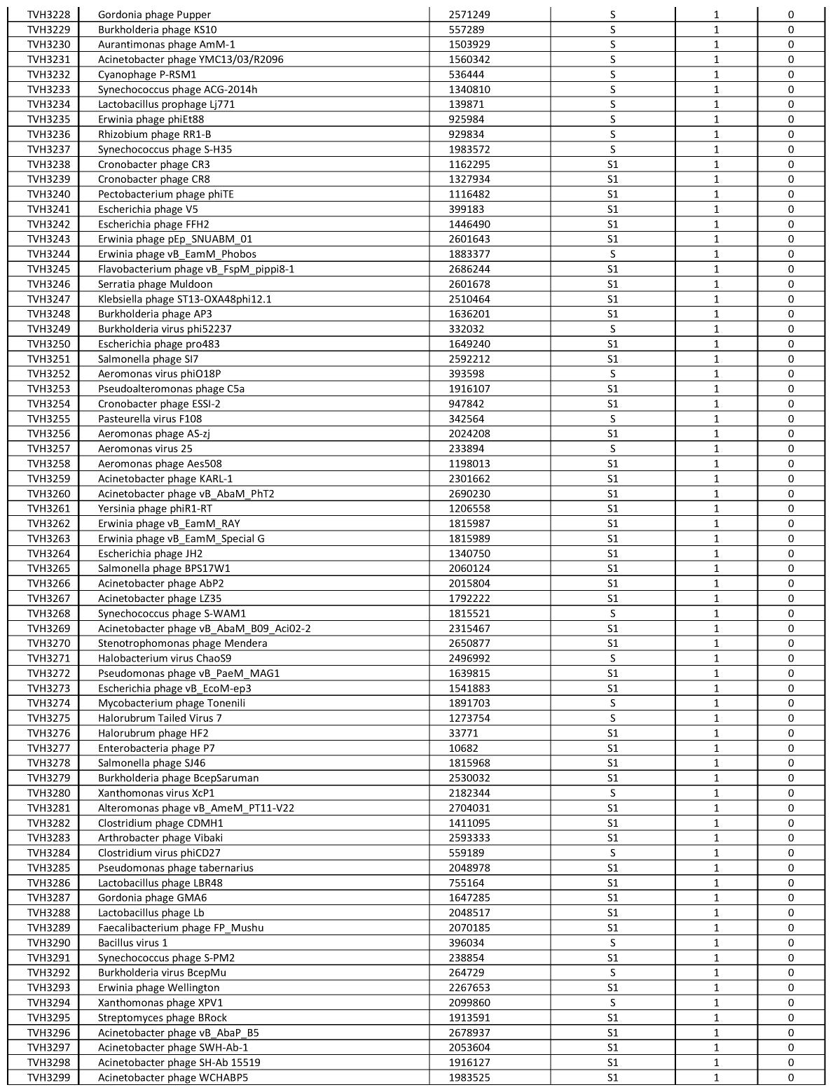

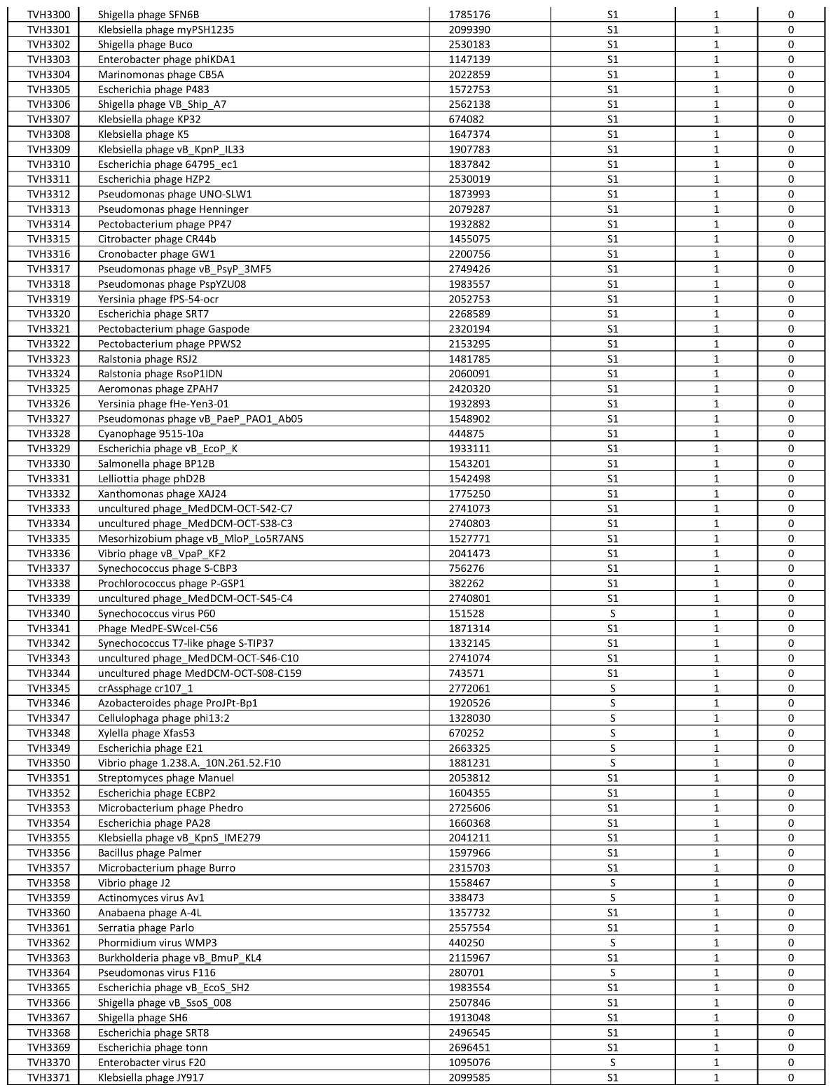

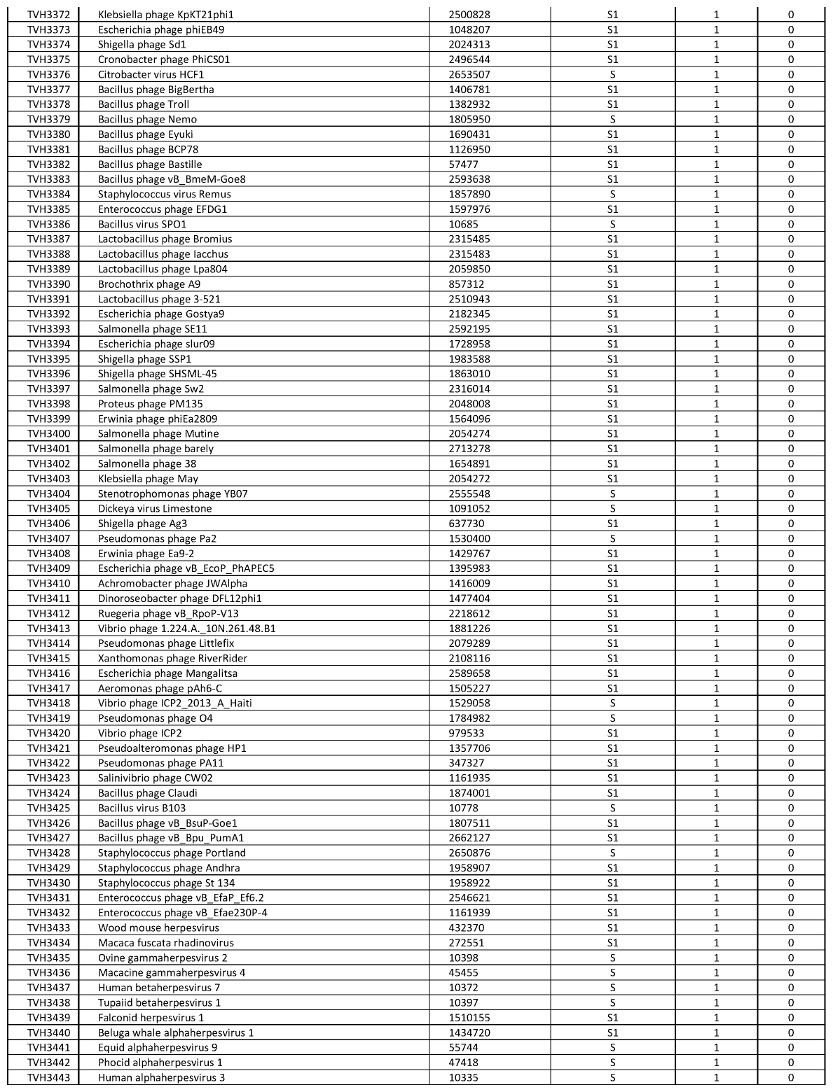

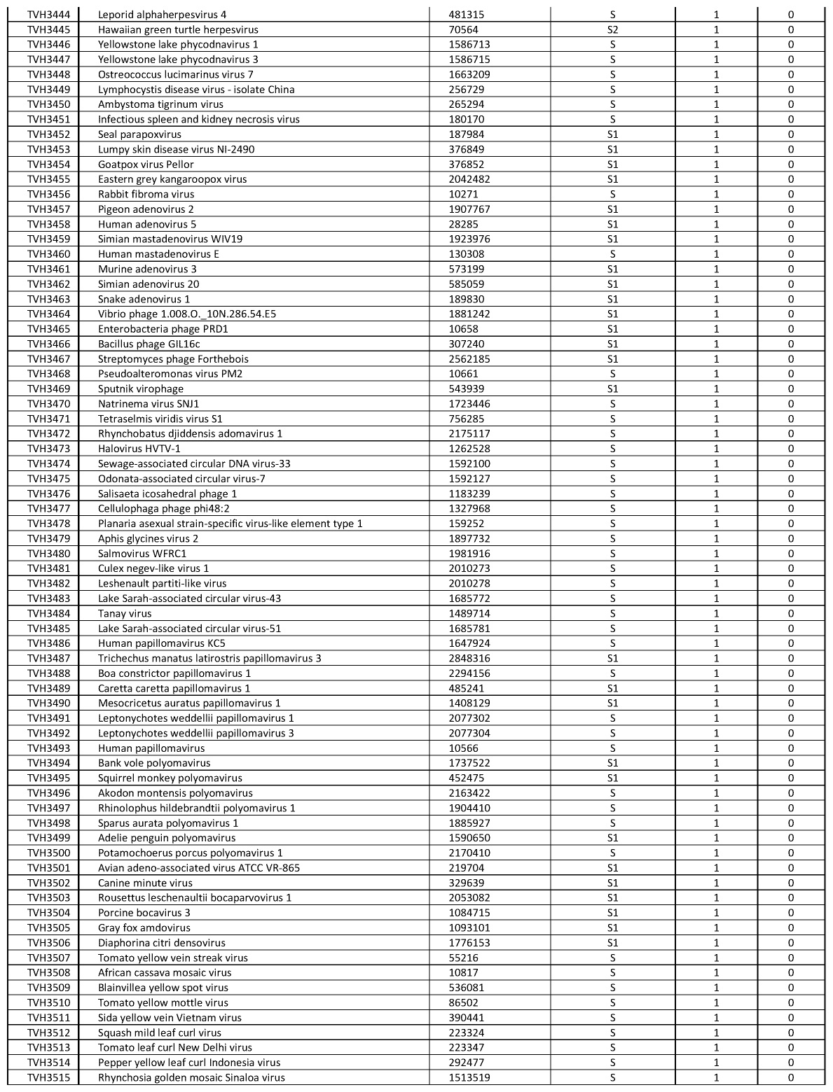

In some embodiments the nucleic acid sequences are derived from sequencing transcripts derived from the BiPS by Iso-seq. Exemplary Iso-Seq derived sequences are set forth in SEQ ID NO: 1-7. The sequences can be classified using Kraken 2. Exemplary Kraken 2 classification of Iso-Seq derived sequences and bat genome sequences are presented in TABLE 2. Exemplary full-length retrovirus sequence identified are RFe-V-MD1, RFe-V-MD2 RFe-V-MD3 RFe-V-MD4, and RFe-V-MD5, set forth in SEQ ID NO: 1-7. A detailed analysis of the sequence of RFe-V-MD1 is shown in FIG. 9D, showing the location of the Env, Pol, and Gag proteins in the genome. A detailed analysis of RFe-V-MD2 sequences is shown in FIG. 9E. The sequences comprise Columbid/Falconid herpesvirus and Sindbis virus sequences as shown. Detailed alignments of exemplary protein sequences are shown in FIG. 11A. A detailed analysis of RFe-V-MD3 sequences show similarities with HKHD40, HKNPC60, human respiratory synscytial virus and SARS-CoV2 (FIG. 9G). Detailed alignments of exemplary protein sequences of the SARS-CoV2 similar sequence with the sequence of a SARS-CoV2 virus isolated from a patient is shown in FIG. 11C. A detailed analysis and comparison of RFe-V-MD4 sequences with Scotophilus bat coronavirus spike protein is shown in FIG. 9H.

In some embodiments, exemplary nucleic acid sequences and an alignment with known viruses such as Scotophilus bat coronavirus 512 are shown in TABLE 3 and RaTG13 bat coronavirus are shown in TABLE 4.

FIG. 11B shows alignments of sequences identified to be similar to Lymphocystis disease virus and Erythocytic necrosis virus.

Other viral sequences such as presented in TABLE 3 and TABLE 4, or SEQ ID NO: 1-349 can be identified. Translated into amino acid sequences, and aligned with known viral sequences as described herein.

### III. Antigens and T Cell Epitopes

Methods for identifying antigens (e.g., antigens derived from an infectious disease organism) include identifying antigens that are likely to be presented on a cell surface (e.g., presented by MHC on an infected cell or an immune cell, including professional antigen presenting cells such as dendritic cells), and/or are likely to be immunogenic. As an example, one such method may comprise the steps of: obtaining at least one of exome, transcriptome or whole genome nucleotide sequencing and/or expression data from an infected cell or an infectious disease organism (e.g., RFe-V-MD1, RFe-V-MD2 RFe-V-MD3 RFe-V-MD4, and RFe-V-MD5, Columbid/Falconid herpesvirus, and Sindbis virus), wherein the nucleotide sequencing data and/or expression data is used to obtain data representing peptide sequences of each of a set of antigens (e.g., antigens derived from the infectious disease organism); inputting the peptide sequence of each antigen into one or more presentation models to generate a set of numerical likelihoods that each of the antigens is presented by one or more MHC alleles on a cell surface, such as an infected cell of the subject, the set of numerical likelihoods having been identified at least based on received mass spectrometry data; and selecting a subset of the set of antigens based on the set of numerical likelihoods to generate a set of selected antigens. Antigens can include nucleotides or polypeptides. For example, an antigen can be an RNA sequence that encodes for a polypeptide sequence. Antigens useful in vaccines can therefore include nucleotide sequences or polypeptide sequences. Antigens can be selected that are predicted to be presented on the cell surface of a cell, such as an infected cell or an immune cell, including professional antigen presenting cells such as dendritic cells. Antigens can be selected that are predicted to be immunogenic. Exemplary antigens predicted using the methods described herein to be presented on the cell surface by an MHC include predicted MHC class I epitopes and predicted MHC class II epitopes. Exemplary nucleic acid sequences or polypeptide sequences for antigen prediction are presented in SEQ ID NO: 1-349, FIGS. 9D-9H and FIGS. 11A-11C, TABLE 3 and TABLE 4.

Protein sequences for the desired antigen are analyzed for potential HLA specific antigens by using for example the SYFPEITHI algorithm (Rammensee et al. (1999) Immunogenetics 50:213-219), and the artificial neural network (ANN) and stabilized matrix method (SMM) algorithms from IEDB (Peters et al. (2005) PLoS Biol. 3:e91). Peptides are selected based on a predicted binding value of either >21 for SYFPEITHY, <6000 for ANN, or <600 for SMM. Selected peptides are synthesized.

Binding assays can be performed using a fluorescence polarization (FP) assay as previously described (e.g., Buchi et al. (2004) Biochemistry 43:14852-14863; Sette et al. (1994) Mol. Immunol. 31:813-822). To determine binding capacity of the peptides, percentage inhibition relative to controls can be determined in an FP competition assay with the placeholder peptide.

In some embodiments, the peptides bound to the pMHC multimers are from an unbiased library of peptides derived from the antigen. In some embodiments, the peptides are 9-mers. In some embodiments, the peptides bound to the pMHCI multimers are 9-mers which include an HLA-A2 binding motif with key amino acids at positions 2 and 9 which can include isoleucine (I), valine (V) or leucine (L).

In some embodiments, the library comprises all k-mer peptides produced by transcription and translation of any polynucleotide sequence of interest, for example, in silico production of the transcription and translation products of both the forward and reverse strands of a genome or metagenome in all six reading frames.

In some embodiments, a library of the disclosure comprises all k-mer peptides that can be derived from in silico translation of an exome of interest. In some embodiments, a library of the disclosure comprises all k-mer peptides that can be derived from in silico translation of a transcriptome of interest. In some embodiments, a library of the disclosure comprises all k-mer peptides that can be derived from a proteome of interest. In some embodiments, a library of the disclosure comprises all k-mer peptides that can be derived from in silico translation of an ORFeome of interest. In some embodiments, an algorithm can be used to select peptides in a peptide library. For example, an algorithm can be used to predict peptides most likely to fold or dock in an MHC/HLA binding pocket, and peptides above a certain threshold value can be selected for inclusion in the library.

In some embodiments, a library of the disclosure comprises all peptides that can be derived from in silico transcription and translation or translation of a group of genomes, proteomes, transcriptomes, ORFeomes, or any combination thereof. In some embodiments, the peptides are derived from in silico transcription and translation or translation of polynucleotide sequences from a group of samples, for example, clinical samples from a patient population, or a group of pathogen genomes.

One or more polypeptides encoded by an antigen nucleotide sequence can comprise at least one of: a binding affinity with MHC with an IC50 value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more antigens can be presented on the surface of an infected cell (e.g., a., RFe-V-MD1, RFe-V-MD2 RFe-V-MD3 RFe-V-MD4, and RFe-V-MD5, Columbid/Falconid herpesvirus, or Sindbis virus infected cell).

One or more antigens can be immunogenic in a subject having or suspected to have an infection (e.g., a RFe-V-MD1, RFe-V-MD2 RFe-V-MD3 RFe-V-MD4, and RFe-V-MD5, Columbid/Falconid herpesvirus, or Sindbis virus infection), e.g., capable of eliciting a T cell response or a B cell response in the subject. One or more antigens can be immunogenic in a subject at risk of an infection (e.g., a RFe-V-MD1, RFe-V-MD2 RFe-V-MD3 RFe-V-MD4, and RFe-V-MD5, Columbid/Falconid herpesvirus, or Sindbis virus infection), e.g., capable of eliciting a T cell response or a B cell response in the subject that provides immunological protection (i.e., immunity) against the infection, e.g., such as stimulating the production of memory T cells, memory B cells, or antibodies specific to the infection.

One or more antigens can be capable of eliciting a B cell response, such as the production of antibodies that recognize the one or more antigens (e.g., antibodies that recognize a RFe-V-MD1, RFe-V-MD2 RFe-V-MD3 RFe-V-MD4, and RFe-V-MD5, Columbid/Falconid herpesvirus, and Sindbis virus antigen and/or virus). Antibodies can recognize linear polypeptide sequences or recognize secondary and tertiary structures. Accordingly, B cell antigens can include linear polypeptide sequences or polypeptides having secondary and tertiary structures, including, but not limited to, full-length proteins, protein subunits, protein domains, or any polypeptide sequence known or predicted to have secondary and tertiary structures. In general, antigens capable of eliciting a B cell response to an infection are antigens found on the surface of an infectious disease organism (e.g., RFe-V-MD1, RFe-V-MD2 RFe-V-MD3 RFe-V-MD4, and RFe-V-MD5, Columbid/Falconid herpesvirus, and Sindbis virus). Exemplary antigens capable of eliciting a B cell response include, but are not limited to, ORF1ab, spike (S), envelope (E), membrane (M), and nucleocapsid (N).

One or more antigens that induce an autoimmune response in a subject can be excluded from consideration in the context of vaccine generation for a subject.

The size of at least one antigenic peptide molecule (e.g., an epitope sequence) can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the antigenic peptide molecules are equal to or less than 50 amino acids.

Antigenic peptides and polypeptides can be: for MHC Class I 15 residues or less in length and usually consist of between about 8 and about 11 residues, particularly 9 or 10 residues; for MHC Class II, 6-30 residues, inclusive.

In some embodiments, a recombinant cell is provided comprising a nucleic acid or polypeptide set forth in SEQ ID NO: 1-349. The recombinant cells can be used in therapeutic development, such as vaccines, small molecules and biologics. In some embodiments, a recombinant cell is provided comprising a nucleic acid or protein or part thereof set forth in FIG. 9D-9H and FIG. 11A-11C, TABLE 3, and TABLE 4. In some embodiments, the recombinant cell expresses a protein encoded by the nucleic acid or a portion thereof acid or a polypeptide set forth in SEQ ID NO: 1-349. In some embodiments, the recombinant cell expresses a protein encoded by the nucleic acid or a portion thereof acid set forth in FIGS. 9D-9H and FIGS. 11A-11C, TABLE 3, and TABLE 4. In some embodiments the recombinant cell is used to assay for suitable antigens. In some embodiments the recombinant cell is used to produce a selected antigen.

### IV. Pharmaceutical Compositions

The present disclosure also features pharmaceutical compositions that contain a therapeutically effective amount of one or more T cell epitopes, nucleic acids coding for T cells epitopes or peptides. The composition can be formulated for use in a variety of drug delivery systems. One or more physiologically acceptable excipients or carriers can also be included in the composition for proper formulation.

In various embodiments, the pharmaceutical compound includes an acceptable pharmaceutically acceptable carrier. The carrier(s) should be “acceptable” in the sense of being compatible with the other ingredients of the formulations and not deleterious to the subject. Pharmaceutically acceptable carriers include buffers, solvents, dispersion media, coatings, isotonic and absorption delaying agents, and the like, that are compatible with pharmaceutical administration. In one embodiment the pharmaceutical composition is administered orally and includes an enteric coating suitable for regulating the site of absorption of the encapsulated substances within the digestive system or gut.

Pharmaceutical compositions containing a therapeutic, such as those disclosed herein, can be presented in a dosage unit form and can be prepared by any suitable method. A pharmaceutical composition should be formulated to be compatible with its intended route of administration. Useful formulations can be prepared by methods well known in the pharmaceutical art. For example, see Remington's Pharmaceutical Sciences, 18th ed. (Mack Publishing Company, 1990).

Pharmaceutical formulations, in some embodiments, are sterile. Sterilization can be accomplished, for example, by filtration through sterile filtration membranes. Where the composition is lyophilized, filter sterilization can be conducted prior to or following lyophilization and reconstitution.

### Vaccines

Disclosed herein is an immunogenic composition, e.g., a vaccine composition, capable of raising a specific immune response, e.g., a tumor-specific immune response. Vaccine compositions typically comprise a plurality of viral antigens, e.g., selected using a method described herein. Vaccine compositions can also be referred to as vaccines.

The viral nucleic acids, proteins, antigens, and T cell epitopes can be used to design prophylactic or therapeutic vaccines comprising such composition (e.g., pharmaceutical compositions) for immunizing subjects at risk of contracting, or subjects having already contacted, a virus set forth in TABLE 1 or TABLE A. In certain embodiments, the vaccine is a subunit vaccine. In certain embodiments, the vaccine elicits a protective immune reaction against a plurality of viruses (e.g., RFe-V-MD1, RFe-V-MD2 RFe-V-MD3 RFe-V-MD4, or RFe-V-MD5). In certain embodiments, the vaccine elicits a protective immune reaction against a virus set forth in TABLE 1 or TABLE A.

In some embodiments, the vaccine comprises a recombinant nucleic acid molecule comprising one or more promoter and a nucleic acid encoding for a T cell epitope. In some embodiments the nucleic acid is set forth in SEQ ID NO: 1-349, TABLE 3, TABLE 4, or a functional portion thereof.

A vaccine composition of the disclosure can comprise a peptide composition(s) comprising the T cell epitope(s). Alternatively, a vaccine composition of the disclosure can comprise a nucleic acid composition, e.g., an RNA composition or DNA composition, encoding the T cell epitope(s). For such nucleic acid vaccines, suitable regulatory sequences are included such that the peptide epitope is expressed from the nucleic acid (RNA or DNA) in cells of the subject being immunized. In some embodiments, the nucleic acids or the peptides are synthetic.

A vaccine can contain between 1 and 30 peptides, 2, 3, 4, 5, 6, 7, 8, 9, 10, 11, 12, 13, 14, 15, 16, 17, 18, 19, 20, 21, 22, 23, 24, 25, 26, 27, 28, 29, or 30 different peptides, 6, 7, 8, 9, 10 11, 12, 13, or 14 different peptides, or 12, 13 or 14 different peptides. Peptides can include post-translational modifications. A vaccine can contain between 1 and 100 or more nucleotide sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different nucleotide sequences, 6, 7, 8, 9, 10 11, 12, 13, or 14 different nucleotide sequences, or 12, 13 or 14 different nucleotide sequences. A vaccine can contain between 1 and 30 viral antigen sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different viral antigen sequences, 6, 7, 8, 9, 10 11, 12, 13, or 14 different viral antigen sequences, or 12, 13 or 14 different viral antigen sequences.

In some embodiments, the pharmaceutical composition comprises a plurality of (e.g., 2, 3, 4, 5, 6, 7, 8, 9, 10, or more) proteins or peptides and a pharmaceutically acceptable carrier or excipient. A pharmaceutical composition comprising a nucleic acid encoding the mRNA of claim 44 or the protein or peptide of any one of claims 46-48 and a pharmaceutically acceptable carrier or excipient.

In some embodiments, the pharmaceutical composition comprises one or more nucleic acids encoding a plurality of (e.g., 2, 3, 4, 5, 6, 7, 8, 9, 10, or more) mRNAs and a pharmaceutically acceptable carrier or excipient.

In one embodiment, antigens or T cell epitopes are for example ORF1ab, spike (S), envelope (E), membrane (M) and nucleocapsid (N), RNA polymerases, kinases, and viral proteases. Exemplary antigens are shown in FIG. 9D-9H and FIG. 11A-11C, exemplary nucleic acids encoding antigens or portions of antigens are set forth in TABLE 3 and TABLE 4.

In certain embodiments, the two or more of the T cell peptides collectively recognize MHC molecules in at least 50%, at least 60%, at least 70%, at least 80%, at least 90%, at least 95%, or at least 99% of the human population. In certain embodiments, the vaccine contains individualized components according to the personal need (e.g., MHC variants) of the particular patient.

In one embodiment, different peptides and/or polypeptides or nucleotide sequences encoding them are selected so that the peptides and/or polypeptides capable of associating with different MHC molecules, such as different MHC class I molecule. In some aspects, one vaccine composition comprises coding sequence for peptides and/or polypeptides capable of associating with the most frequently occurring MHC class I molecules. Hence, vaccine compositions can comprise different fragments capable of associating with at least 2 preferred, at least 3 preferred, or at least 4 preferred MHC class I molecules.

The vaccine composition can be capable of raising a specific cytotoxic T-cell response and/or a specific helper T-cell response.

A vaccine composition of the disclosure can comprise one or more short (e.g., 8-35 amino acids) peptides as the immunostimulatory agent. In certain embodiments, a cell surface antigen sequence is incorporated into a larger carrier polypeptide or protein, to create a chimeric carrier polypeptide or protein that comprises the T cell epitope(s). This chimeric carrier polypeptide or protein can then be incorporated into the vaccine composition.

Recombinant cells can be engineered to express proteins and peptides of the disclosure. Vectors can be designed for the expression of cell surface antigens (e.g. nucleic acid transcripts, proteins, or enzymes) in prokaryotic or eukaryotic cells. For example, cell surface antigens can be expressed in bacterial cells such as Escherichia coli, insect cells (using baculovirus expression vectors), yeast cells, or mammalian cells. Suitable host cells are discussed further in Goeddel (1990) Gene Expression Technology: Methods in Enzymology 185, Academic Press, San Diego, Calif. The cell surface antigens can be purified from the recombinant cells and used in antibody development or further formulated into pharmaceutical compositions. Additionally or alternatively, the recombinant cells expressing the cell surface antigens can be used for producing antibodies or T cells specific to the cell surface antigens.

It is understood that a peptide can be expressed from a nucleic acid (e.g., an mRNA) in a cell of the subject. Exemplary methods of producing peptides by translation in vitro or in vivo are described in U.S. Patent Application Publication No. 2012/0157513 and He et al., J. Ind. Microbiol. Biotechnol. (2015) 42(4):647-53. The present disclosure provides a composition (e.g., pharmaceutical composition) comprising one or more nucleic acids (e.g., mRNAs) encoding one or more cell surface antigens or derived peptides. It is understood that a peptide can be expressed from a nucleic acid (e.g., an mRNA) in a cell of the subject. Exemplary methods of producing peptides by translation in vitro or in vivo are described in U.S. Patent Application Publication No. 2012/0157513 and He et al., J. Ind. Microbiol. Biotechnol. (2015) 42(4):647-53. The present disclosure provides a composition (e.g., pharmaceutical composition) comprising one or more nucleic acids (e.g., mRNAs) encoding one or more peptides disclosed herein, optionally further comprising a pharmaceutically acceptable carrier or excipient. In certain embodiments, the composition comprises nucleic acid sequences encoding two or more (e.g., three or more, four or more, five or more, six or more, seven or more, eight or more, nine or more, ten or more, 11 or more, 12 or more, 13 or more, 14, or more, 15 or more, 16 or more, 17 or more, 18 or more, 19 or more, or 20 or more) of the peptides disclosed herein. In certain embodiments, the two or more peptides are derived from the same cell surface antigen. In certain embodiments, the two or more peptides are derived from at least two different cell surface antigens. In certain embodiments, the two or more peptides collectively are recognized by MHC molecules in at least 50%, at least 60%, at least 70%, at least 80%, at least 90%, at least 95%, or at least 99% of the human population. In certain embodiments, the vaccine contains individualized components according to the personal need (e.g., MHC variants) of the particular patient. In certain embodiments, each of the nucleic acids further comprises one or more expression control sequences (e.g., promoter, enhancer, translation initiation site, internal ribosomal entry site, and/or ribosomal skipping element) operably linked to one or more of the peptide coding sequences.

A vaccine composition can further comprise an adjuvant and/or a carrier. Examples of useful adjuvants and carriers are given herein below. A composition can be associated with a carrier such as e.g. a protein or an antigen-presenting cell such as e.g. a dendritic cell (DC) capable of presenting the peptide to a T-cell.

Adjuvants are any substance whose admixture into a vaccine composition increases or otherwise modifies the immune response to a viral antigen. Carriers can be scaffold structures, for example a polypeptide or a polysaccharide, to which a viral antigen, is capable of being associated. Optionally, adjuvants are conjugated covalently or non-covalently.

The ability of an adjuvant to increase an immune response to an antigen is typically manifested by a significant or substantial increase in an immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to 1018 ISS, alum, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives can readily be determined by the skilled artisan without undue experimentation. Additional adjuvants include colony-stimulating factors, such as Granulocyte Macrophage Colony Stimulating Factor (GM-CSF, sargramostim).

A vaccine composition of the disclosure can comprise one or more short (e.g., 8-35 amino acids) peptides as the immunostimulatory agent. In certain embodiments, a T cell epitope sequence is incorporated into a larger carrier polypeptide or protein, to create a chimeric carrier polypeptide or protein that comprises the T cell epitope(s). This chimeric carrier polypeptide or protein can then be incorporated into the vaccine composition.

A vaccine composition can comprise more than one different adjuvant. Furthermore, a therapeutic composition can comprise any adjuvant substance including any of the above or combinations thereof. It is also contemplated that a vaccine and an adjuvant can be administered together or separately in any appropriate sequence.

A carrier (or excipient) can be present independently of an adjuvant. The function of a carrier can for example be to increase the molecular weight of in particular mutant to increase activity or immunogenicity, to confer stability, to increase the biological activity, or to increase serum half-life. Furthermore, a carrier can aid presenting peptides to T-cells. A carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. However, tetanus toxoid and/or diptheria toxoid are suitable carriers. Alternatively, the carrier can be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is possible if a trimeric complex of peptide antigen, MHC molecule, and APC (antigen presenting cell) is present. Correspondingly, it may enhance the immune response if not only the peptide is used for activation of CTLs, but if additionally APCs with the respective MHC molecule are added. Therefore, in some embodiments a vaccine composition additionally contains at least one antigen presenting cell.

Viral antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more viral antigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, infected cells express the viral antigens, and thereby elicit a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of viral antigens, e.g., Salmonella typhi vectors, and the like will be apparent to those skilled in the art from the description herein. In some embodiments, the viral vector is a adenovirus vector.

The compositions (e.g., pharmaceutical compositions) disclosed herein may be formulated for delivery into cells (e.g., APCs, such as dendritic cells, monocytes, macrophages, or artificial APCs). In certain embodiments, the composition comprises an agent that facilitate transfection in vitro or in vivo, such as a liposome or a nanoparticle (e.g., lipid nanoparticle). In certain embodiments, the liposome or nanoparticle further comprises a binding moiety (e.g., an antibody or an antigen-binding fragment thereof) for delivering the liposome or nanoparticle to a target T cell (e.g., a professional APC). Another delivery method employs virus particles (e.g., adenovirus, adeno-associated virus, vaccinia virus, fowlpox virus, self-replicating alphavirus, marabavirus, or lentivirus). In certain embodiments, the composition comprises a pharmaceutically acceptable carrier or excipient, such as a diluent, an isotonic solution, water, etc. Excipients also can be selected for enhancement of delivery of the composition.

Suitable routes of administration and dosages for vaccines are known in the art and can be determined by a person of medical skill. In certain embodiments, the vaccine is administered parenterally, e.g., by intramuscular, intradermal, subcutaneous, intravenous, topical, nasal, or local administration. In certain embodiments, the vaccine comprising peptide(s) is administered via skin scarification. In certain embodiments, the vaccine comprising peptide(s) is administered at a dosage of 0.1-10 mg, e.g., 0.1-0.5 mg, 0.5-1 mg, 1-3 mg, 1-5 mg, or 5-10 mg of total amount per human patient. In certain embodiments, the vaccine comprises a plurality of different peptides, wherein each peptide is provided at a dosage of 0.01-0.05 mg, 0.05-0.1, or 0.1-0.5 mg per human patient. Stimulation of an anti-virus T cell immune response in a subject by the vaccine can be monitored by methods established in the art, e.g., by isolating T cells from the subject and measuring reactivity of the T cells to the viral T cell epitope(s) contained within the vaccine (see, e.g., Immunohistochemistry, ELISPOT, binding assays such as Biacore and ELISA, and LC-MC techniques).

### Small Molecule Drugs

Small molecule drug therapeutics generally refer to therapeutics of low molecular weight (e.g., below 1 kDa) that modulate cellular behavior to treat a disease. Such small molecule drugs bind one or more biological targets of a target cell, thereby causing a change in the activity or function of the biological target of the target cell. Given their size, small molecule drug therapeutics are able to penetrate cellular membranes, thereby enabling them to bind or affect biological targets located within cells.

In various embodiments, small molecule drug therapeutics are inhibitors that serve to inhibit a biologic target that is involved in a disease. For example, small molecule drug therapeutics may be kinase inhibitors, proteasome inhibitors, proteinase inhibitors, or protein inhibitors. Additionally, small molecule drug therapeutics can be chemotherapeutics that prevent cell replication such as alkylating agents, anti-microtubule agents, topoisomerase inhibitors, DNA intercalators, and the like.

More comprehensive lists of small molecule drug therapeutics are found in publicly available databases such as DrugBank, ChemSpider, ChEMBL, KEGG, and PubChem. In some embodiments, the small molecule is an inhibitor of a protein or portion thereof encoded by the nucleic acid sequence set forth in SEQ ID NO: 1-349. In some embodiments, the small molecule is an inhibitor of a protein or portion thereof set forth in FIG. 9D-9H and FIG. 11A-11C, or encoded by the nucleic acid sequence or a portion thereof set forth in TABLE 3 and TABLE 4.

### Biologics

Biologics generally refer to therapeutics that are manufactured from biologic sources (e.g., produced in cells). Biologics are larger than small molecule drugs and often times more complex in structure and molecular makeup. In various embodiments, biologics are synthesized through manufacturing methods that include 1) inserting a DNA sequence encoding for the biologic or a portion of the biologic into a living cell, 2) having the cell produce transcribe/translate the DNA sequence into a protein, 3) isolating the protein from the cells, where the protein serves as the biologic or a component of the biologic. Example of biologics include antibodies (e.g., monoclonal or polyclonal antibodies), cytokines, growth factors, enzymes, immunomodulators, recombinant proteins, vaccines, allergenics, blood components, hormones, therapeutic cells (e.g., stem cells), tissues, carbohydrates, and nucleic acids.

### V. Kits

In some embodiments, any of the BiPS or viral sequences disclosed herein is assembled into a pharmaceutical or diagnostic or research kit to facilitate their use in therapeutic, diagnostic or research applications. A kit may include one or more containers housing any of the vectors, nucleic acids, proteins, peptides, or viruses disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure. For example, it will be appreciated that all features described and depicted herein can be applicable to all aspects of the disclosure described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the embodiments remain operable. Moreover, two or more steps or actions may be conducted simultaneously.

The use of any and all examples, or exemplary language herein, for example, “such as” or “including,” is intended merely to illustrate better the embodiments and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1 Isolation of Bat Embryonic Fibroblasts

This example describes the isolation of embryonic fibroblasts from bats. An embryo (approximately developmental stage 20) acquired from a Spanish Rhinolophus ferrumequinum bat (wild horseshoe bat) was cut into several pieces while removing the head and as much as the inner organ tissue as possible. The pieces were then flushed with PBS and processed separately. The tissue was covered with 0.05% trypsin, minced with a scalpel, and incubated in a cell culture incubator at 37° C. and 5% CO₂ for 45 minutes. The trypsin was deactivated with fibroblast medium consisting of DMEM (Life Technologies, CA), 10% fetal bovine serum (Sigma, MO), 0.1 mM MEM Non-essential amino acids (Life Technologies, CA), 2 mM GlutaMax supplement (Life Technologies, CA), and Penicillin-Streptomycin (10 U/ml and 10 μg/ml, respectively; Life Technologies, CA). The cells were broken up by pipetting up and down 20 times, collected by centrifugation, transferred to a gelatin-coated (Sigma-Aldrich, MO) T75 cell culture treated flasks (Corning, AZ) in 15 ml of fibroblast medium, and cultured at 37° C. and 5% CO₂. After 3 days, when reaching ˜80% confluency, the attached cells were washed with DPBS (Life Technologies, CA), treated with 0.05% trypsin-EDTA, (Life Technologies, CA) to obtain a single cell solution and either split at a ratio of 1:4 or used directly in a reprogramming experiment.

### Example 2 Isolation of Bat Fibroblasts from Tail Biopsies

This example describes the isolation of fibroblasts from tail biopsies from adult bats.

M. myotis bats were sampled in Morbihan, Brittany in North-West France in accordance with the permits and ethical guidelines issued by ‘Arrêté’ by the Préfet du Morbihan and the University College Dublin ethics committee. This population has been transponded and followed since 2010 as part of on-going mark-recapture studies by Bretagne Vivante and the Teeling laboratory (Huang et al., 2019). Once captured, all bats were placed in individual cloth bags before processing. A single 3 mm biopsy was taken from the outstretched uropatagium of each bat using a sterile biopsy punch and immediately submerged in a Cryotube with 2 ml of DMEM cell culture medium supplemented with 20% FBS, 1% NEA, and 1% Antibiotic-Antimycotic containing Streptomycin, Amphotericin B and Penicillin, maintaining as sterile conditions as possible. All bats were offered food and water and rapidly released after processing. Biopsies were then stored at 4° C. and transported to the laboratory for processing within 6 days. Samples were further processed through a cell extraction methodology similar to a previously established protocol (Kacprzyk et al., 2021) with a few modifications. The samples were rinsed with DPBS and cut finely within a minimal amount of cell culture medium using sterile blades to result in six 0.5 mm pieces. These pieces were then transferred aseptically to a cryotube containing cell culture medium and incubated for 18 hours with collagenase type II at 37° C. with 5% CO₂ to allow for digestion. The pieces were collected by centrifugation for 5 minutes at 300 rcf, resuspended in 2 ml of fresh cell culture medium and transferred to a 35 mm cell culture treated plate for initial P1 expansion. Cells were then fed every 2-3 days with cell culture medium as above but a reduced 0.2% concentration of antibiotic-antimycotic. For the first feeding a % media change was performed to avoid sudden changes in antibiotic-antimycotic concentration from 1% to 0.2%. When the cells reached 70% confluency, they were transferred to a T25 flask in cell culture medium after treatment with 0.05% Trypsin and were fed every 2-3 days as necessary. At 85% confluency, the cells were trypsinized as before and 1×10\hat{ }6 cells were frozen in 1 ml cell culture medium containing 10% DMSO.

### Example 3 Reprogramming and Expansion of Bat Embryonic and Adult Fibroblasts into Bat iPSCs

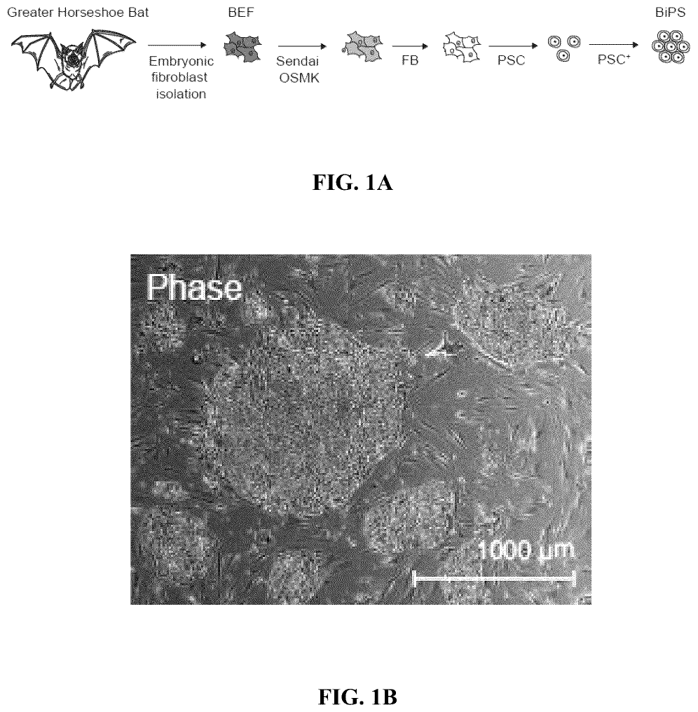

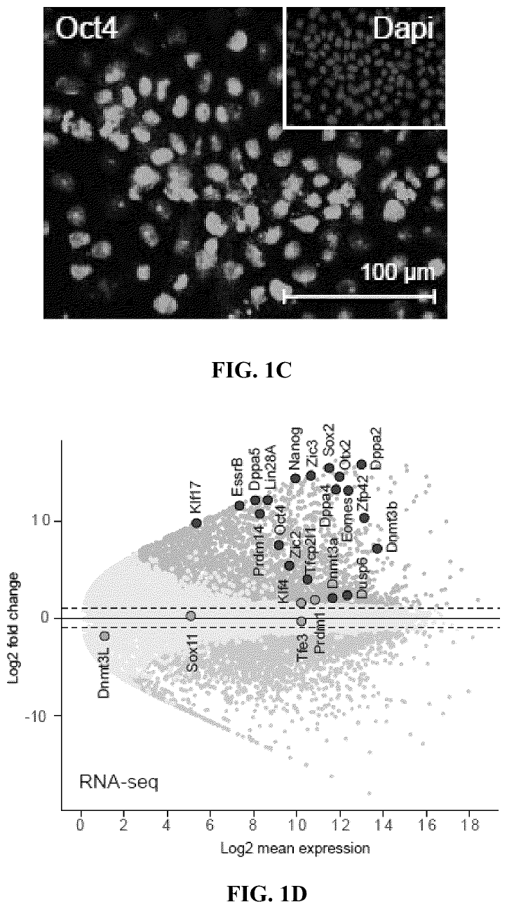

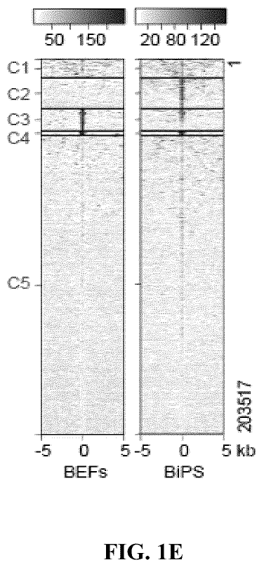

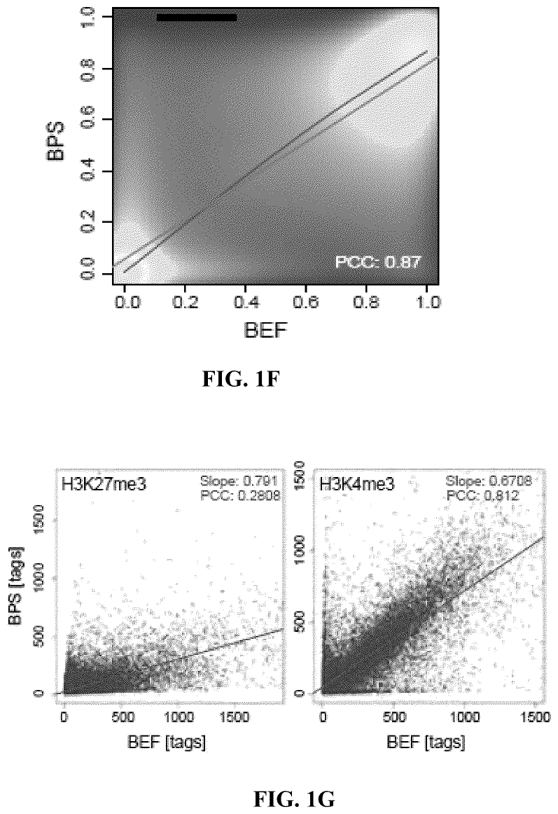

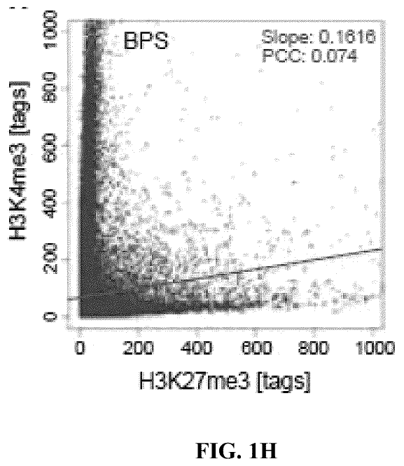

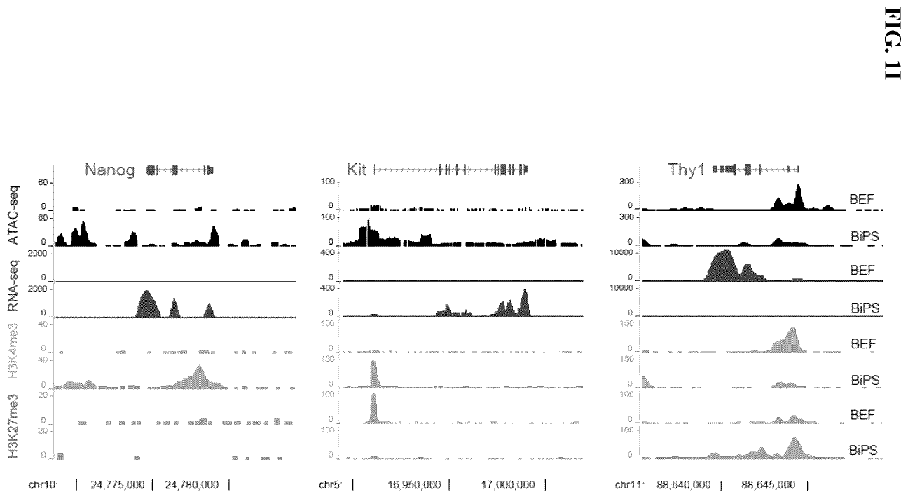

This example describes the reprogramming of bat embryonic fibroblasts for the generation of bat iPSCs. First, the original Yamanaka reprogramming protocol (Takahashi et al., Cell (2006) 126, 663-676) based on four reprogramming factors (Oct4, Sox2, Klf4, and cMyc) was tried, because it provides the most direct way to generate pluripotent stem cells in most species. Strikingly, the standard protocol that is highly effective in mice, humans and other mammalian species (domestic dog, (Canis familiaris), domestic pig, (Sus scrofa), common marmoset (Callithrix jacchus)) failed in bats. Even though the standard reprogramming protocol failed, it provided the crucial insight that the Yamanaka factors triggered the formation of rudimentary stem cell-like colonies even though the reprogrammed cells ceased to expand. Thus, the core pluripotency network might be conserved in bats. However, the signaling cascades that usually shield this network from differentiation cues are different. An exemplary bat pluripotent stem cell derivation strategy is illustrated in FIG. 1A.

Briefly, 150,000 embryonic Rhinolophus ferrumequinum fibroblasts at passage 2, adult Myotis myotis at passage 3, or CF1 mouse embryonic fibroblasts at passage 3 were resuspended in 1 ml of fibroblast medium and mixed with Sendai-virus particles containing the reprogramming factors Oct4, Sox2, cMyc, and Klf4 (CytoTune iPS 2.0, Life Technologies, CA) with a final multiplicity of infection (MOI) of 10, 10, 10, and 15, respectively. The cells were plated on one gelatin-coated well of a 6-well plate and cultured at 37° C. with 5% CO₂. The medium was replaced every 24 hours. 6 days after transduction, the cells of each well were collected by treatment with 0.05% trypsin-EDTA, seeded at a density of 50,000 cells per 60 cm² on irradiated CF1 mouse embryonic fibroblasts (MEFs; ThermoFisher, MA) in fibroblast medium. After 24 hours, the medium was switched to 50% fibroblast medium and 50% pluripotent stem cell (PSC) medium consisting of DMEM/F-12 (Life Technologies, CA), 20% knockout serum replacement, 0.1 mM MEM Non-essential amino acids, 2 mM GlutaMax supplement, Penicillin-Streptomycin (10 U/ml and 10 μg/ml, respectively), 100 μM 2-mercaptoethanol, and 40 ng/ml FGF2. From then on, the medium was replaced every day with PSC medium until day 14 when the FGF concentration was increased to 100 ng/ml and the medium was supplemented with 10\hat{ }4 U/ml Leukemia inhibitory factor (Lif), 100 ng/ml SCF (R&D Systems, MN) and 20 nM Forskolin Forskolin. Colonies appeared 14 to 16 days after transduction, were picked on day 20 and expanded on irradiated MEFs with Gentle Cell dissociation Reagent (StemCell Technologies, MA). After that, cells were passaged approximately every 5 days, or when they were confluent, at a ratio of 1:6 to 1:12 onto irradiated MEFs. Cell and colony morphology were recorded with an EVOS digital inverted microscope (Invitrogen, MA).

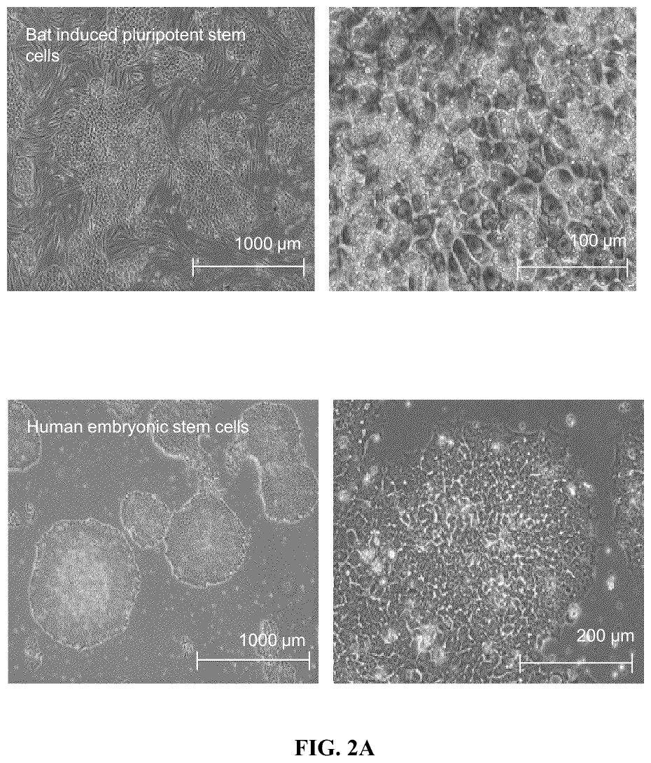

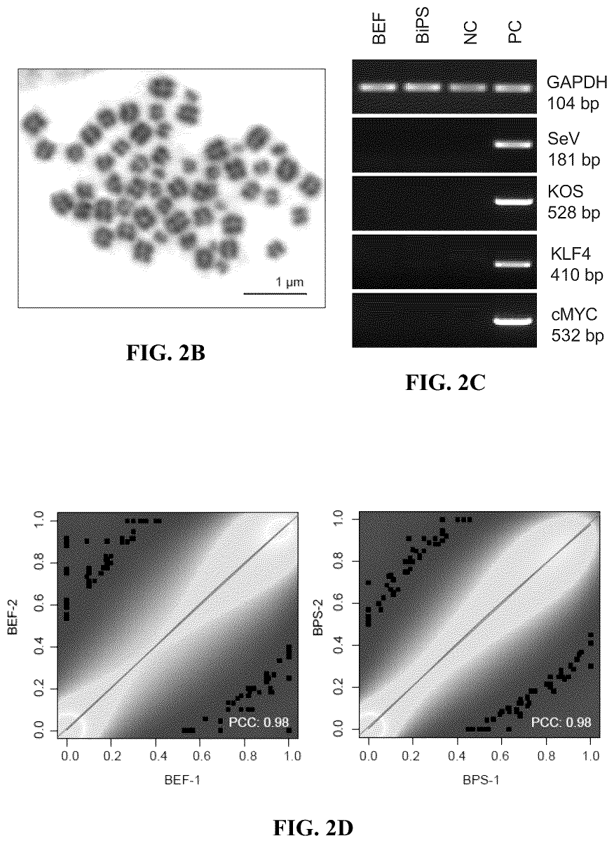

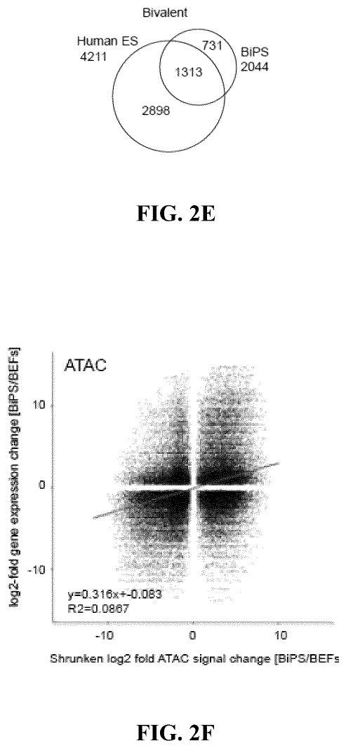

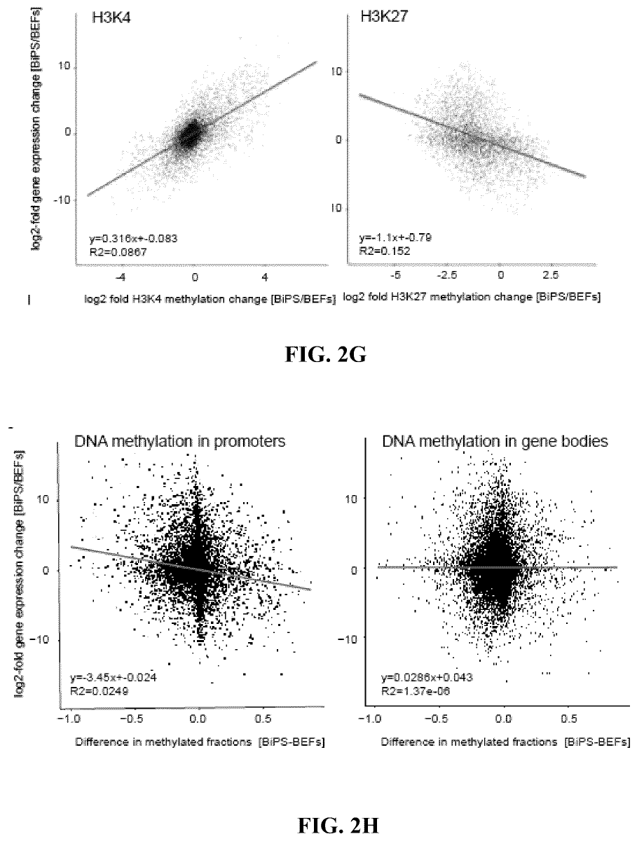

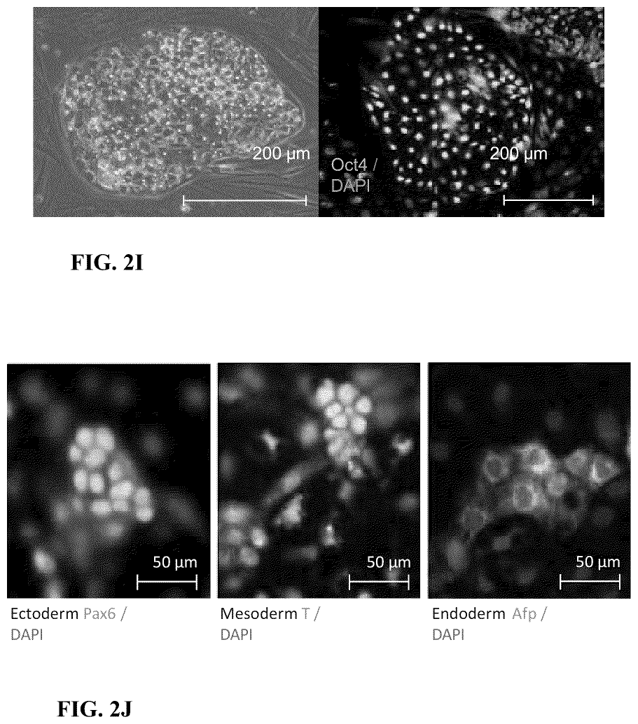

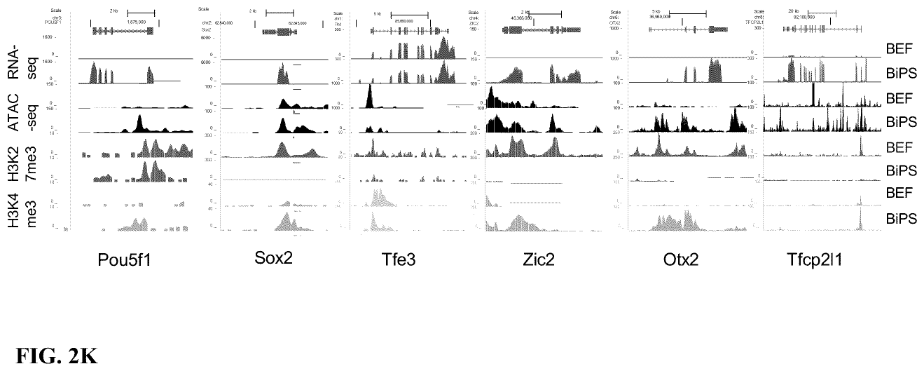

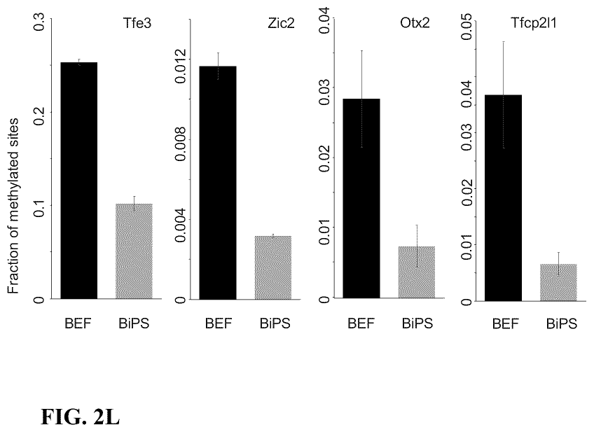

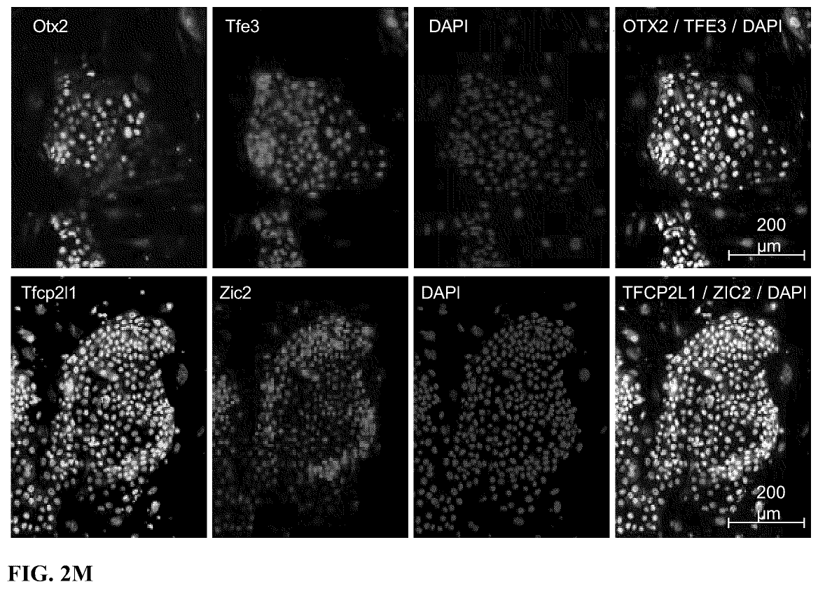

Thus, specific ratios of reprogramming factors, and the addition of Lif, Scf, the Pka activator forskolin and Fgf2 to the culture medium allowed for the uninterrupted growth of bat pluripotent stem cells. Under these conditions, bat stem cell colonies typically appeared after 14-16 days of culture. These initial stem cell colonies were, however, not readily pickable and expandable using conventional EDTA- (Versene), collagenase- or trypsin-based methods that are normally used to passage pluripotent stem cells from other species. To split cells for further passaging and growth cells were lightly flushed off the feeder cell layer after gentle treatment with low concentrations of EDTA. Exemplary cell morphology of the reprogrammed bat iPSCs is shown in FIG. 1B and FIG. 2A. Bat pluripotent stem cell colonies appeared tight and homogeneous. The cells had a large, apparent nucleus with one or two prominent nucleoli. Their proliferation rate was similar to human pluripotent cells despite a somewhat lower clonogenicity. The iPSC reprogramming protocol was further validated by developing iPS cells from an evolutionary distant bat species Myotis myotis (greater mouse-eared bat) non-lethally sampled in the wild, which exhibited similar attributes to the greater horseshoe bat iPS cells, suggesting that this unique pluripotent state evolved in the ancestral bat lineage. The iPSC cells derived from the M. myotis tail cell show that these fibroblasts were also readily reprogrammable using the new ‘batified’ Yamanaka protocol and yielded similar bat iPSCs that were Oct4 positive in immunostaining and differentiated into all three germ layers (FIG. 2I-J), suggesting that the protocol is applicable across the deepest basal divergencies in bats.

### Example 4 Characterization of the Reprogrammed Cells

This example illustrates the characterization of the reprogrammed cells. After reprogramming, cells were analyzed for karyotype, chromatin organization, and gene and RNA expression.

### Karyotyping

This example illustrates the karyotyping of reprogrammed cells. Briefly, cells were treated with 100 ng/ml KaryMax Colcemid Solution in HBSS (Life Technologies, CA) for 16 hours, then treated with 0.05% trypsin-EDTA for 15 minutes and filtered through a 40 μm cell strainer to remove clumps. Cells were collected by centrifugation, resuspended in 1 ml 0.075 M potassium chloride (Sigma-Aldrich, MO) and incubated for 20 minutes at room temperature. 0.5 ml fixative (1 part glacial acetic (Fisher Scientific, MA) mixed with 3 parts methanol (Sigma-Aldrich, MO) were added, cells were collected as before, resuspended in 4 ml fixative, and incubated for 20 minutes at room temperature. The fixation step was repeated, the cells collected as before and all but about 200 μl of the fixative was removed. The cells were resuspended in the remaining fixative and dropped onto slides that were precooled at −20° C. The slides were airdried and the cells stained for 10 minutes with Giemsa Staining solution consisting of 1 part KaryoMax Giemsa solution (Life Technologies, CA) and 3 parts Gurr buffer (Invitrogen, MA). The slides were washed with water, dried, and mounted in Cytoseal 60 (Thermo Scientific, MA). High-resolution pictures of chromosome spreads were acquired with an AxioObserver microscope (Zeiss) using the 100× oil objective. Even after prolonged culture (over 50 passages), the cells retained a normal karyotype, with most cells containing 56 chromosomes (FIG. 2B).

### RT-PCR

mRNA was extracted with the RNeasy Mini Kit (Qiagen). 500 ng of each sample were used to generate cDNA by reverse transcription using the SuperScript™ IV VILO™ Master Mix (Invitrogen). 2 μl of the cDNA were used to detect the presence of Sendai virus transcripts using GoTaq Green Polymerase (Promega), and the oligos as recommended in the CytoTune iPS 2.0 kit (Invitrogen). Gapdh was amplified as loading control using oligos with the following sequence: Z25-132:GAPDH_F1_GHB: TGGTGAAGGTCGGAGTGAAC (SEQ ID NO: 350) and Z25-133:GAPDH_R1_GHB: GAAGGGGTCATTGATGGCGA (SEQ ID NO: 351)). The PCR products were analyzed on a 2% agarose gel containing ethidium bromide.

### Immunofluorescence Staining

For immunofluorescence staining, cells were plated on pt-slides (Ibidi, Germany). After 4 days, cells were washed once with DPBS and fixed with Cytofix/Cytoperm solution (Becton Dickinson, NJ) for 20 minutes at 4° C. Cells were rinsed with Perm/Wash buffer (Becton Dickinson, NJ) and then incubated overnight at 4° C. in Perm/Wash buffer containing primary anti-Afp (R&D Systems, MN) anti-Pax6 (BioLegend, CA), J2 anti-dsRNA (Scicons, Hungary), anti-(gag/pol) HERVK (Austrial Biological) or FIPV3-70 anti-Pan Corona (Life Technologies, CA) or directly conjugated anti-Oct3/4-AF488 (Santa Cruz, CA) or anti-Brachyury (R&D Systems, MN) anti-Otx2 (R&D Systems), anti-Zic2 (Abcam), anti-Tfe3 (Sigma Aldrich) or anti-Tfcp2l1 (R&D Systems) in a 1:50 (anti-Oct3/4) or 1:100 dilution (all others). Cells were rinsed and washed 3 times for 2 minutes with Perm/Wash solution at room temperature followed by a 1-hour incubation with a 1:200 dilution of the corresponding secondary antibodies (Donkey anti-chicken-Cy3, Millipore, AP194C; Goat anti-chicken-AF488; Donkey anti-rabbit-AF647; Goat anti-rabbit-AF488, Goat anti-mouse-AF488) in Perm/Wash buffer. Cells were rinsed, washed twice for 2 minutes with Perm/Wash Buffer and then incubated for 5 minutes with Perm/Wash buffer containing 2 drops per ml NucBlue Dapi stain (Invitrogen, MA). The buffer was removed, and the cells were cover-slipped in Prolong Dimond antifade mounting medium (Invitrogen, MA). Images were acquired with an AxioObserver fluorescence microscope with Apotome (Zeiss). For the simulated emission depletion (STED) microscopy (super-resolution), the cells were plated on coverslips that were placed in wells of 6-well plates. The staining was performed as described above but with a 1:200 dilution of the Abberior Star 635P secondary antibody in Perm/Wash buffer. Cells were rinsed, washed twice for 2 minutes with Perm/Wash Buffer and then incubated for 5 minutes with Perm/Wash buffer containing 2 drops per ml DyeCycle Violet stain. The coverslips were mounted face down on glass slides with Prolong Dimond antifade mounting medium (Invitrogen). Images were acquired with a TCS SP8 confocal microscope with STED 3× and White Light Laser (Leica) with a 100× oil objective. 405 nm and 594 nm lasers were used for excitation and 775 nm laser for depletion. Image resolution obtained was 19.8 μm by 19.8 μm using a zoom factor of 6×. Exemplary immunofluorescent detection of Oct4/Pou5f2 in BiPS cells shows that the cells were positive for the pluripotency factor Oct4 (FIG. 1C).

### RNA Isolation and RNA-Seq

For RNA-seq, RNA was extracted from BiPS cells at passage 22 and BEFs at passage 3. RNA was extracted with the RNeasy RNA isolation kit (Qiagen, Germany) following the manufacturer's recommendations including the DNase digest (Qiagen, Germany) and eluted in 50 μl RNase/DNase free H₂O. The libraries were prepared with the SMART-Seq v4 Ultra Low Input kit (Takara Bio, undifferentiated cells) or the Stranded Total RNA with Ribo-Zero Plus kit (Illumina, differentiated cells) and 100 bp paired-end sequencing reads were (PE100) were generated by Illumina sequencing (NovaSeq 6000 S1) to a depth of 50 million reads (100 million total reads).

### RNA-Seq Mapping and Visualization

The quality of the reads from the RNA sequencing was analysed with FastQC v0.11.9 (Andrews, 2010), and visualized using MultiQC (Ewels et al., 2016. With the mean phred score of around Q35 across each base position no filter or processing was performed. To carry out the differential expression analysis, the genome of Rhinolophus ferrumequinum was used as reference genome, RefSeq assembly accession GCF_004115265.1, assembled and annotated by the Vertebrate Genomes Project (www.vertebrategenomesproject.org). The reads were mapped with HISAT2 v2.2.1 (Kim et al., 2019), the .sam files resulting from each mapping were converted into .bam files and indexed using samtools v1.10 (Li et al., 2009). The reads were mapped against each gene using featureCounts v2.0.1 (Liao et al., 2014) and the differential expression analysis was performed with DESeq2 v1.10.1 (Love et al., 2014). To visualize the RNA-seq data in the UCSC genome browser, bigwig files were generated using the bamCoverage command from deepTools (www.deeptools.readthedocs.io/en/develop/content/tools/bamCoverage.html; Ramirez et al., 2016).

### MA Plot

The MA plots were generated based on the DESeq2 (see above) results with the ggmaplot function (www.rpkgs.datanovia.com/ggpubr/reference/ggmaplot.html) from the R package ggpubr (www.rpkgs.datanovia.com/ggpubr/). Genes are indicated by dots, plotted by their log 2 fold change between bat fibroblast and pluripotent stem cells and the log 2 mean of normalized counts (ratio of means). Blue dots indicate genes with an adjusted p value of (or FDR) of <0.05 and a fold change of 2 (log 2 fold change of 1), red dots indicate genes with an adjusted p value (or FDR) of <0.05 and fold change of −2 (log 2 fold change of −1). Dotted lines are drawn at fold change of 2/−2 (log 2 fold change of 1/−1).

RNA-seq analyses revealed the induced expression of canonical pluripotency-associated genes (FIG. 1D).

However, closer data inspection revealed that the expression profile did not necessarily match any known pluripotency state. Instead, factors indicative of the so-called naive pluripotent state (Klf4, Klf17, Essrb, Tfcp2l1, Tfe3, Dppa, and Dusp6) were expressed alongside genes typically found in the more advanced primed pluripotent cells (e.g., Otx2, Zic2). Double immunostainings detecting four of the most commonly used primed/naïve factors, Otx2/Tfe3 and Tfcp2l1/Zic2, respectively, showed co-expression of naïve and primed markers in most cells (FIGS. 2K-M). No methylation in the promoters of Nanog, Pou5f1, or Sox2 was detected, which might be related to under-annotation of the Rhinolophus ferrumequinum genome at this point in time Germ cell factors such as Dnmt3l and Dazl were absent. Thus, while cellular heterogeneity might be at play, their uniform appearance makes it most likely that bat stem cells occupy a novel, yet-to-be-characterized pluripotent default state.

### ATAC-Seq

To analyze the effects of the reprogramming approach on the bat chromatin and epigenetic structures a global epigenetic landscape survey using ATAC-seq was performed. ATAC-seq and bioinformatics analysis to detect open chromatin in bat fibroblasts and bat pluripotent stem cells was performed by Active Motif, CA from 100,000 cryopreserved cells (ATAC-seq service). In brief, nuclei were isolated and libraries of open chromatin were prepared with the Nextera Library Prep Kit (Illumina) by Tn5 tagmentation. The tagmented DNA was purified using the MinElute PCR purification kit (Qiagen, Germany), amplified with 10 cycles of PCR, and purified using Agencourt AMPure SPRI beads (Beckman Coulter, CA). 42 bp paired-end sequencing reads (PE42) were generated by Illumina sequencing (using NextSeq 500) to a depth of at least 83 million total reads and mapped to the GCA_004115265.2 genome (Ensembl, annotation version 102) using the BWA algorithm with default settings (“bwa mem”). Alignment information for each read was stored as BAM file. Only reads that passed the Illumina's purity filter, aligned with no more than 2 mismatches, and mapped uniquely to the genome were used in the subsequent analysis. Duplicate reads (“PCR duplicates”) were removed. Genomic regions with high levels of transposition/tagging events were then determined using the MACS2 peak calling algorithm (Zhang et al., Genome Biology (2008) 9:R137). To identify the density of transposition events along the genome, the genome was divided into 32 bp bins and the number of fragments in each bin was determined. The data were then normalized by reducing the tag number of all samples by random sampling to the number of tags present in the smallest sample. Peak metrics between samples were compared by grouping overlapping Intervals into “Merged Regions,” which are defined by the start coordinate of the most upstream Interval and the end coordinate of the most downstream Interval (=union of overlapping Intervals; “merged peaks”). In locations where only one sample has an Interval, this Interval defines the Merged Region. Intervals and Merged Regions, their genomic locations along with their proximities to gene annotations and other genomic features were determined and average and peak (i.e. at “summit”) fragment densities were compiled. The sequencing tracks (number of fragments in each 32 bp bin stored as .bigwig file) were visualized with the UCSC genome browser.

The global epigenetic landscape survey using ATAC-seq revealed significant chromatin configuration changes when bat fibroblasts transitioned into the pluripotent state (FIG. 1E). Generally, there were strict correlations between newly opened sites and gene expression and conversely closed regions and gene shutdowns (FIG. 1F). Similarly, mapping the DNA methylome by RRBS-seq exposed significant CpG methylation changes across the genome after reprogramming (FIG. 2G-H and).

Reduced Representation Bisulfite Sequencing (RRBS) of Bat iPSCs

Reduced representation bisulfite sequencing of bat fibroblasts and pluripotent stem cells was performed by Active Motif, CA(RRBS Service, Active Motif, CA). Briefly, 500,000 cells were provided as a frozen pellet. Genomic DNA was isolated, and 100 ng were digested with TaqaI (NEB, MA) at 65° C. for 2 hours followed by MspI (NEB, MA) at 37° C. overnight. Following enzymatic digestion, samples were used for library generation with the Ovation RRBS Methyl-Seq System (Tecan, Switzerland) following the manufacturer's instructions. In brief, digested DNA was randomly ligated, and, following fragment end repair, bisulfite converted using the EpiTect Fast DNA Bisulfite Kit (Qiagen, Germany) following the Qiagen protocol. After conversion and clean-up, samples were amplified resuming the Ovation RRBS Methyl-Seq System protocol for library amplification and purification. 75 bp single-end sequencing reads (SE75) were generated by Illumina sequencing (using NextSeq 500) to a depth of at least 27 million reads (total of 54 million reads), with at least 2.9 million covered CpGs. The reads were mapped to the GCA_004115265.2 genome (Ensembl, annotation version 102) and the percentage of methylation at CpG sites across the genome was calculated. To visualize the methylation ratios aligned to the gnome with the UCSC genome browser, the methylation ratio files containing the methylation ratio for each chromosomal position were first converted to bed files, that were then used to generate bigwig files with the bedGraphToBigWig v4 tool (www.encodeproject.org/software/bedgraphtobigwig/). Correlation scatter plots were generated to show the level of methylation at common CpG sites. To visualize the global differences between bat fibroblast and pluripotent stem cells, the RRBS methylation data were combined for all samples based on chromosome position, the ratios of the duplicates were averaged and the methylation ratio for each chromosomal position was plotted using the ggplot2 function “stat_density_2d_filled” with fill based on density. Only chromosomal positions that were present in all replicates were included in the analysis.

Similarly, mapping the DNA methylome by RRBS exposed significant CpG methylation changes across the genome (FIGS. 1A and 2G) after reprogramming.

### Chromatin Immunoprecipitation Sequencing (ChIP-Seq)

5 million cells were fixed cells in 1% formaldehyde by adding 1/10 volume of freshly prepared Formaldehyde Solution (11% formaldehyde, 0.1 M NaCl, 1 mM EDTA, pH 8.0, 50 mM HEPES, pH 7.9) to the existing medium. Cells were agitated for 15 minutes at room temperature and the fixation was stopped by addition of 1/20 volume of 2.5 M glycine solution (final concentration of 0.125 M) to the existing medium and incubation at room temperature for 5 minutes. The cells were scraped off the wells, collected by centrifugation at 800 g and washed with 10 ml chilled 0.5% Igepal in PBS per tube by pipetting up and down. Cells were pelleted by centrifugation as before and resuspended in 10 ml chilled PBS-Igepal containing 1 mM PMSF. Cells were collected as before, and the cell pellet was snap-frozen in liquid nitrogen. Further processing, chromatin immunoprecipitation and bioinformatics analysis to detect H3K4me3 and H3K27me3 was performed by Active Motif, CA(HistoPath ChIP-seq service). In brief, chromatin was isolated by adding lysis buffer, followed by disruption with a Dounce homogenizer. Lysates were sonicated and the DNA sheared to an average length of 300-500 bp with Active Motif's EpiShear probe sonicator. Genomic DNA (Input) was prepared by treating aliquots of chromatin with RNase, proteinase K and heat for de-crosslinking, followed by SPRI beads clean up (Beckman Coulter, CA) and quantitation with Clariostar (BMG Labtech). An aliquot of chromatin (20 μg) was precleared with protein A agarose beads (Life Technologies, CA). Genomic DNA regions of interest were isolated using 4 μg of antibody against H3K4me3 (Active Motif, CA) or H3K27me3 (Active Motif, CA). Complexes were washed, eluted from the beads with SDS buffer, and subjected to RNase and proteinase K treatment. Crosslinks were reversed by incubation overnight at 65° C., and ChIP DNA was purified by phenol-chloroform extraction and ethanol precipitation. Illumina sequencing libraries were generated from the ChIP and Input DNAs with the standard consecutive enzymatic steps of end-polishing, dA-addition, and adaptor ligation. After a final PCR amplification step, 75-nt single-end (SE75) sequence reads were generated by Illumina sequencing (using NextSeq 500) to a depth of at least 36 million reads per sample and mapped to the GCA_004115265.2 genome (Ensembl, annotation version 102) using the BWA algorithm with default settings. Duplicate reads were removed, and only uniquely mapped reads (mapping quality >=25) were used for further analysis. Alignments were extended in silico at their 3′-ends to a length of 200 bp, which is the average genomic fragment length in the size-selected library and assigned to 32-nt bins along the genome. The resulting histograms (genomic “signal maps”) were stored in bigWig files. To find peaks, the generic term “Interval” was used to describe genomic regions with local enrichments in tag numbers. Intervals were defined by the chromosome number and a start and end coordinate. Peak locations were determined using the MACS algorithm (v2.1.0) with a cutoff of p-value=1e-7 (Zhang et al., 2008). Signal maps and peak locations were used as input data to Active Motifs proprietary analysis program, which creates Excel tables containing detailed information on sample comparison, peak metrics, peak locations and gene annotations. No normalization was performed on the H3K27me3 data, while standard normalization was applied to the H3K4me3 data. The tag number of all samples (within a comparison group) was reduced by random sampling to the number of tags present in the smallest sample. To compare peak metrics between 2 or more samples, overlapping Intervals were grouped into “Merged Regions,” which are defined by the start coordinate of the most upstream Interval and the end coordinate of the most downstream Interval (=union of overlapping Intervals; “merged peaks”). In locations where only one sample has an Interval, this Interval defines the Merged Region. The sequencing tracks (number of fragments in each 32 bp bin stored as bigwig file) were visualized with the UCSC genome browser.

ChIP-seq analysis showed that histone marks associated with active (H3K4me3) and developmentally repressed genes (H3K27me3) showed many changes (FIG. 1G, Approximately 18.2% of the bat stem cell genes were associated with a “bi-valent” domain (H3K4me3 and H3K27me3; FIG. 1H), a pluripotency chromatin hallmark initially found in human and mouse pluripotent cells. Interestingly, while there was overlap between human and bat bivalency genes there were also some bat- or human-specific genes (FIG. 2E). Generally, there were strict correlations between newly opened sites and gene expression, and conversely, closed regions and gene shutdowns during the reprogramming process that also corresponded to the absence or presence of histone modifications, respectively (FIG. 1I). However, there are instances when there were simultaneously active and repressive epigenetic marks, most likely as a result of spontaneous differentiation in the cultures (FIG. 2F).

Collectively, the results establish that the bat pluripotent stem cells are reprogrammed both transcriptionally and epigenetically.

### Example 5 Three Germ Layer Differentiation

This example illustrates the further functional characterization of the reprogrammed bat IPS cells. After reprogramming, cells were analyzed in pluripotency assays for pluripotency potential.

The differentiation of bat pluripotent stem cells was carried out with the STEMdiff Trilineage differentiation kit (StemCell Technologies, MA) following the manufacturer's protocol. Cells were plated at the desired densities in mTeSR medium (StemCell Technologies, MA), and plated on Vitronectin-coated (StemCell Technologies, MA) cell culture plates. After 5 days (endoderm or mesoderm) or 7 days (ectoderm) in culture as directed by the manufacturer. For the ectoderm differentiation, the floating three-dimensional structures were then replated and grown for 4 additional days in fibroblast medium. The cells were stained with antibodies detecting the appropriate lineage markers as described above or cells were collected (surface area of 10 cm² per replicate) for RNA isolation and RNAseq after addition of 600 μl lysis buffer RTL (part of the RNeasy kit; Qiagen, Germany).

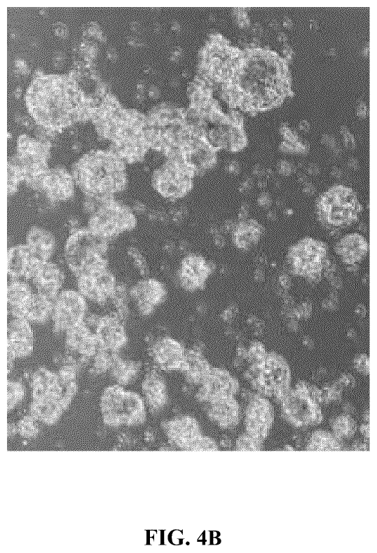

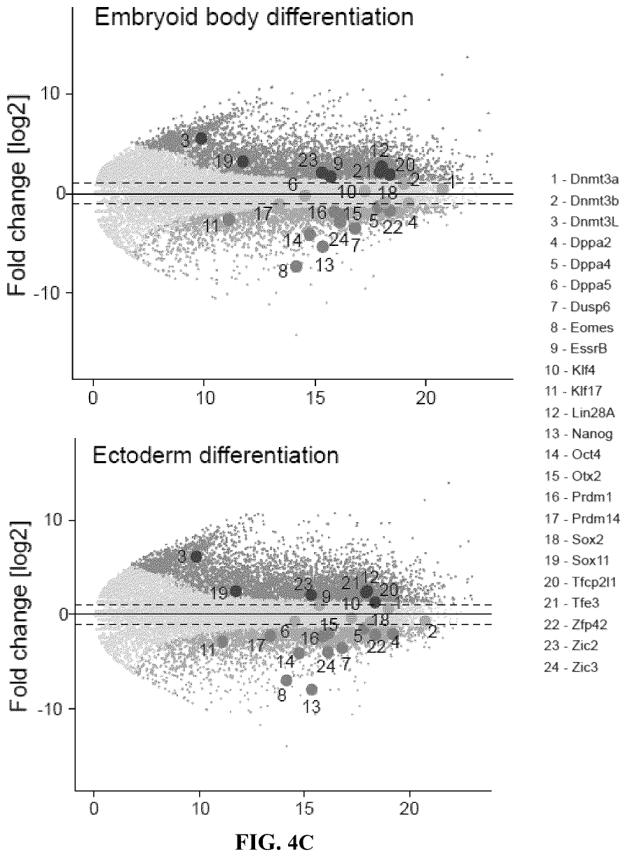

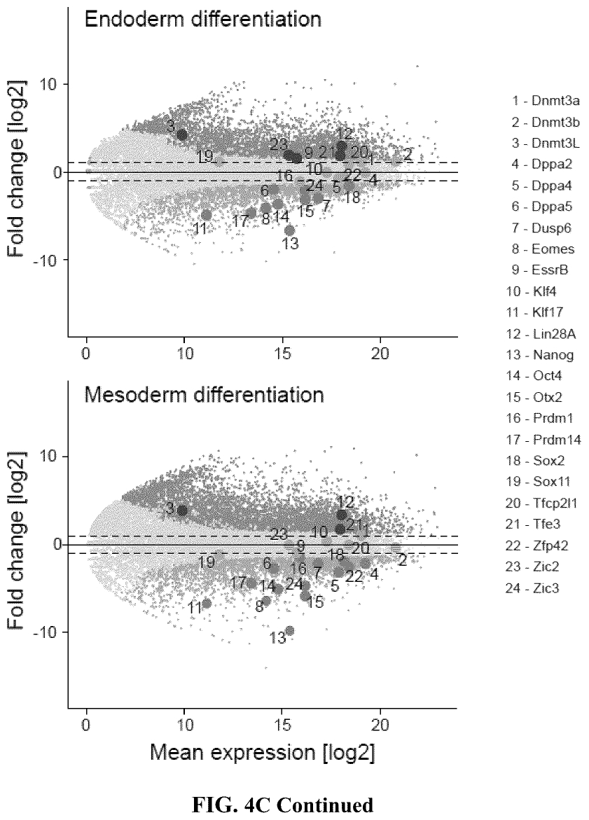

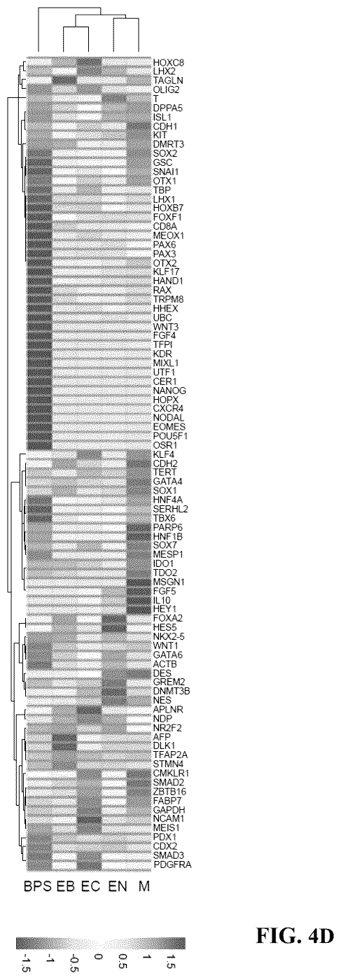

Results show that the bat iPSCs differentiate into ectodermal, mesodermal, and endodermal fates (FIG. 4A). In each case, the cells responded to the altered culture conditions by shifting their morphology profoundly. The differentiated iPSCs turned positive for Pax6 (ectoderm), T (mesoderm) or AFP (endoderm). Since the cells used in this experiment were at an advanced passage (passage 37, an equivalent of about 6 months of continuous culture), the results also suggest that pluripotency can be maintained long-term.

### Embryonic Body Differentiation

To analyze the bat stem cells' developmental plasticity, the cells were subjected to embryoid body (EB) differentiation. Briefly, bat pluripotent stem cells grown on irradiated mouse embryonic fibroblasts from a total area of 60 cm² were washed with PBS, treated for 10 minutes with Gentle Cell Dissociation Reagent (StemCell Technologies, MA), collected by centrifugation and resuspended in 12 ml differentiation medium consisting of DMEM/F-12 (Life Technologies, CA), 10% fetal bovine serum (Sigma, MO), 0.1 mM MEM Non-essential amino acids (Life Technologies, CA), 2 mM GlutaMax supplement (Life Technologies, CA), Penicillin-Streptomycin (10 U/ml and 10 μg/ml, respectively; Life Technologies, 15140122) and 100 μM 2-mercaptoethanol (Fluka, NC). The cells were then transferred to one uncoated 60 cm² petri dish (Corning, 351029). After 3 days in culture, as much as possible of the medium (about ⅔) was carefully exchanged without disturbing and removing the floating EBs that had formed. The floating EBs were collected after 3 more days (total of 6 days) in culture, fixed in Cytofix/Cytoperm fixation buffer (Becton Dickinson, NJ) overnight, and then stained with antibodies against as described above to detect differentiation markers of all three germ-layers by immunofluorescence. For RNA isolation and RNA-seq, EBs were formed as described, collected, resuspended in 6 ml differentiation medium, and distributed into three wells of cell-culture treated 6-well plates (10 cm² each). After 2 more days in culture, the cells were washed with PBS, lysed with 600 μl buffer RTL (part of the RNeasy kit; Qiagen, 74104) and RNA was isolated as described above.

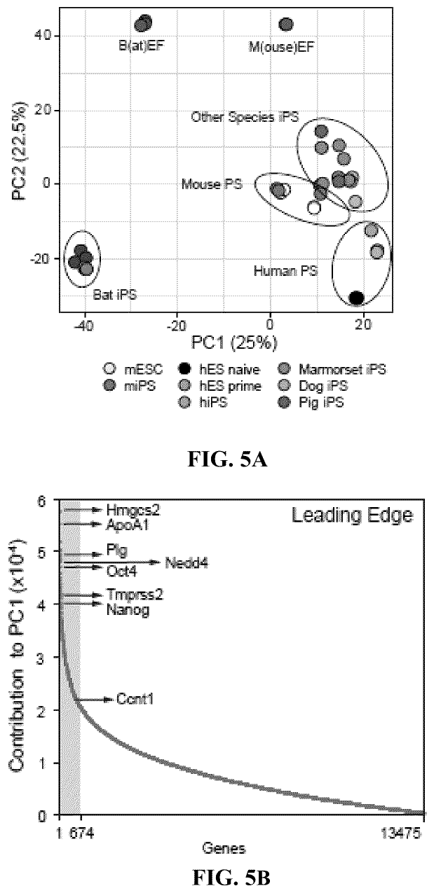

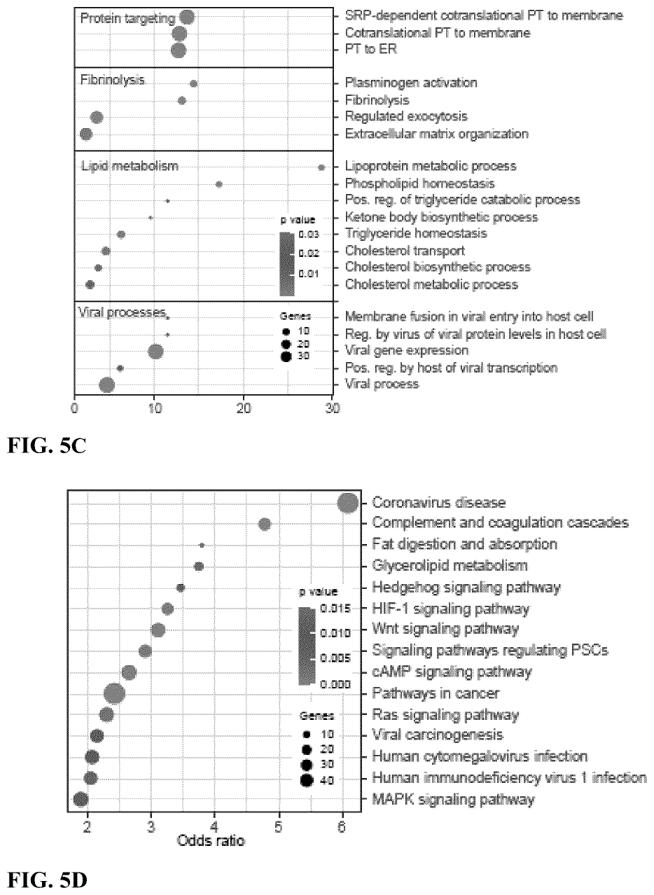

In the assay, cells differentiated and formed the for EBs' typical spherical arrangements. They subsequently matured into elaborate three-dimensional structures that were positive for all three germ layer markers (FIG. 4B). EBs were also analyzed by RNA-seq as described in Example 4. The RNA-seq analysis of RNA isolated from the monolayer differentiation and EB formation confirmed the respective cell fate changes (FIG. 4C, FIG. 5A-D).

### Teratomaformation

To assay the potential of the bat iPSCs to form teratomas in vivo, cells were injected into immunocompromised mice and then analyzed. Briefly, two 6-well plates (12 wells) of bat pluripotent stem cells grown on irradiated mouse embryonic fibroblasts were scraped off in 2 ml DMEM/F-12 medium (Life Technologies, CA), collected by centrifugation and resuspended in 500 μl DMEM/F-12 medium. 100 μl of the cell suspension were injected into the hindleg muscle of 8-week-old male Fox Chase SCID Beige Mice (Charles River, MA). Tumor tissue that had formed after 16 weeks was harvested, fixed in 10% Formalin (Fisher Scientific, MA) overnight and then transferred to 70% ethanol. The tissue was embedded in paraffin and hematoxylin and stained with eosin of 5 μm sections. Images were acquired with an AxioObserver microscope (Zeiss) and analyzed.

The analysis showed, that the bat iPSCs formed a particular tumor (teratoma) at the injection site after four to five months albeit infrequently (33%) and very small (2-4 mm). The tumors were comprised of immature tissue with epithelial, neural and stromal characteristics (FIG. 4D). Transcriptional profiling of pivotal genes previously reported critical for teratoma formation (FIG. 4G) revealed that while some genes are downregulated in bat iPSCs in comparison with mouse iPSCs (like Eras), other genes like the hyaluronidases (HAS) and ADP ribosylation factors (ARFs) are indistinguishable between the experimental groups, making it likely that the anti-tumor effect seen in the rudimentary teratomas is a complex phenomenon. While the host mice were severely immunocompromised and immune-related tissues were not analyzed the immaturity and delay in growth may suggest a yet to be characterized anti-tumorigenic property of bat stem cells similar to, for instance, the naked mole rat which could also underlie the extended healthspans and cancer resistance reported in bats.

### Blastoid Differentiation

To analyze the potential of the iPSCs to form embryoid structures, the cells were subjected to a modified blastoid protocol. Cells were harvested and plated as described for the embryonic body formation above. After 3 days in culture, 100 ng/ml BMP4 (R&D Systems, 314-BP-010) were added to the medium. 24 later the supernatant was diluted with ⅔ of fresh medium and transferred to two fresh uncoated petri dishes. The medium was exchanged after 3 more days in culture and floating blastoids were harvested 4 days later (total of 12 days of differentiation). The blastoids were fixed in Cytofix/Cytoperm fixation buffer (Becton Dickinson, BDB554714) overnight, and stained as described above to detect the expression of Oct4 by immunofluorescence microscopy.

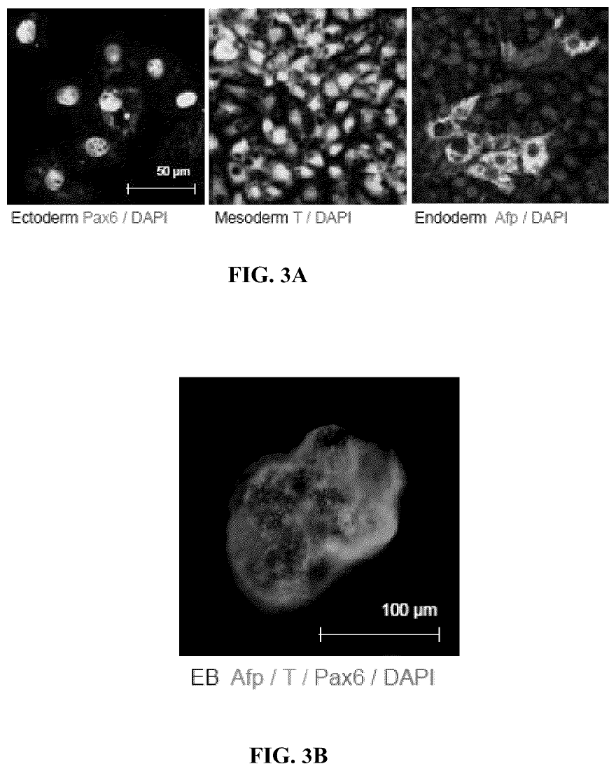

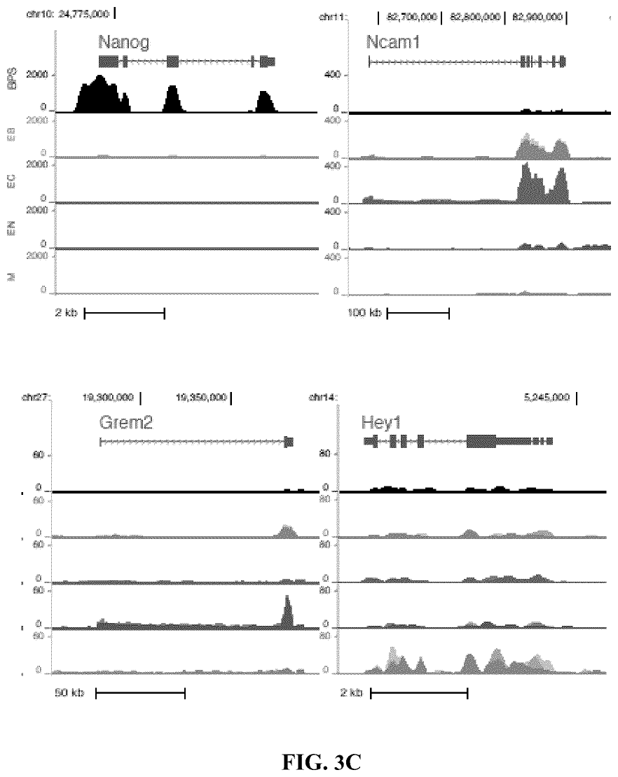

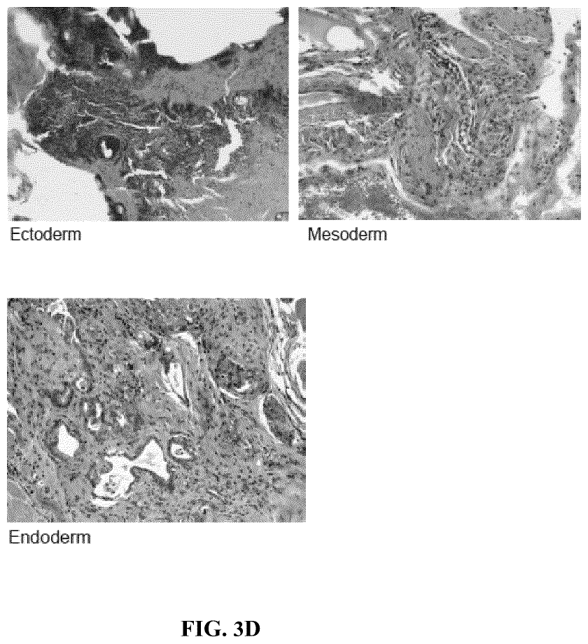

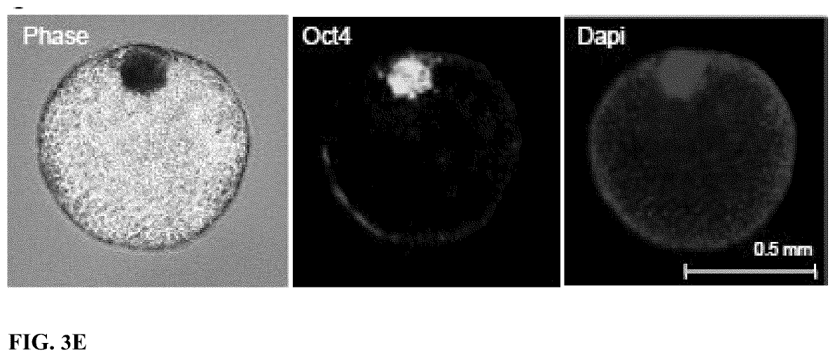

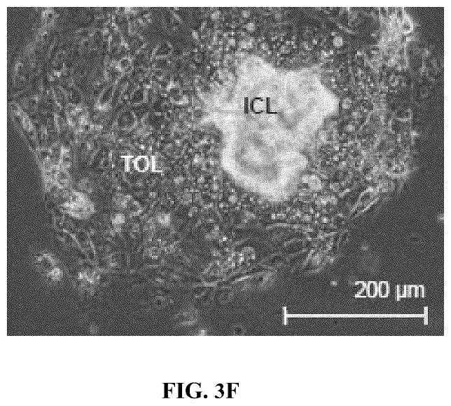

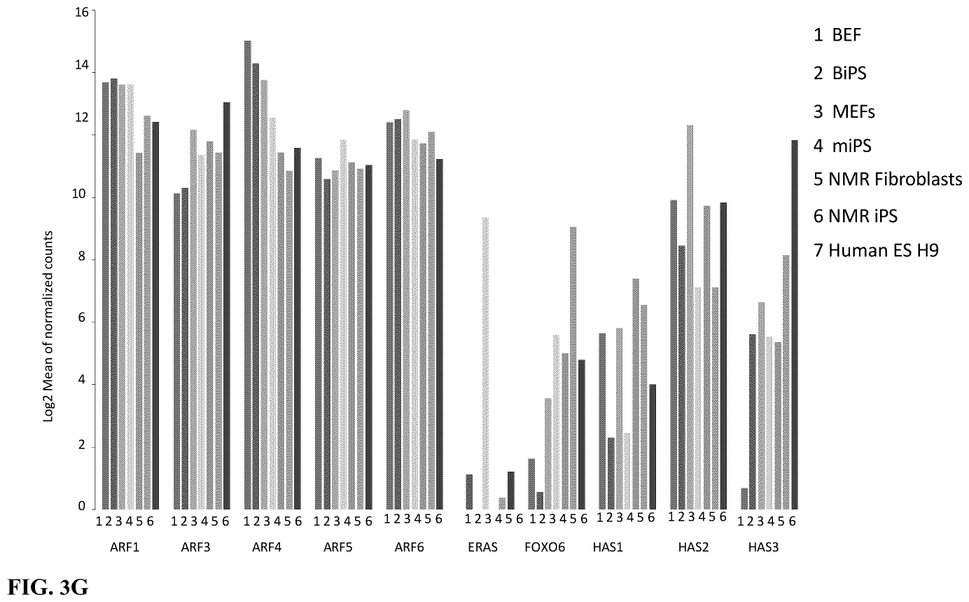

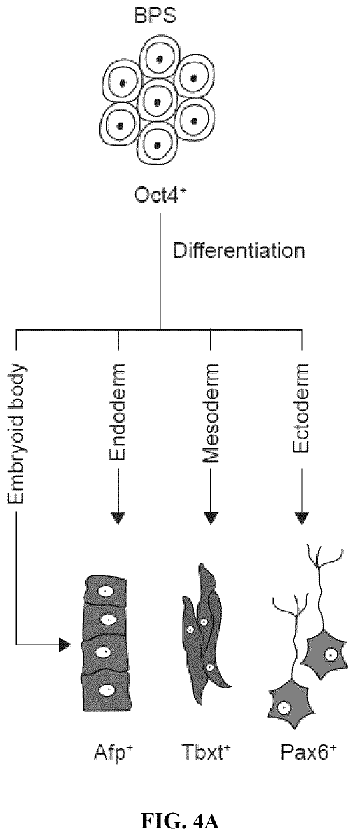

Further analysis showed, that bat blastoids recapitulate critical aspects of preimplantation embryos, including an Oct4-positive inner cell mass, the cystic cavity and a bilayered epithelium consisting of trophoblastic and yolk sac cells (FIG. 3E). Replating these embryo structures resulted in their attachment to a flattened trophoblastic epithelium to grow and an expansion of the inner cell mass (FIG. 3F). These differentiation studies exemplify the unique potential of pluripotent bat cells to recapitulate important developmental events and serve as a powerful model to study the unique physiological adaptations of bats, including their reduced cancer phenotype.

Embryonic stem cell lines were derived from these outgrowths, confirming these embryoids' blastocyst nature.

The differentiation studies exemplify the unique potential of the described pluripotent bat cells to recapitulate important developmental events and serve as a powerful model to study the unique physiological adaptations of bats.

### Example 6 Analysis of the Distinct Characteristics of Pluripotent Bat Stem Cells

To assay distinct characteristics of pluripotent bat stem cells, gene expression patterns in bat stem cells were analyzed such as the ground state transcriptome and then compared to other species. Transcriptome profiles of pluripotent stem cells from an assorted set of species (Bats, mouse, pig, dog, marmoset, human) and different cell types (EF, iPSCs, MEF, ESC) were assembled and principal component analysis was performed to obtain a high-level overview of the number of commonalities and differences between bats and other mammals (FIG. 5A)

### Principal Component Analysis (PCA)

The DESeq2 output files of the RNA-seq analyses described above were subjected to a Variance Stabilizing Transformation (VST) using within-group-variability (Anders and Huber, 2010) to compare the bat pluripotent stem cell transcriptional profile with that of other species. The first two principal components of this result were plotted using the ggscatter function (https://rpkgs.datanovia.com/ggpubr/reference/ggscatter.html) from the R package ggpubr (www.cran.r-project.org/web/packages/ggpubr/index.html). The datasets used in the PCA were: GSM4616525, GSM4616526 and GSM4616527 (dog iPS), GSM4617887, GSM4617889, GSM4617890, GSM4617891, GSM4617895, GSM4617900 and GSM4617901 (marmoset iPS), GSM4616532 (human iPS), GSM4616535 and GSM4616536 (pigIPS) from study GSE152493 (Yoshimatsu et al., 2021), and GSM1287734, GSM1287745 and GSM1287746 (mouse ESC) and GSM1287736, GSM1287747 and GSM1287748 (mouse iPS) from GSE53212 (Carter et al., 2014), as well as GSM2718393 and GSM2718399 (mouse iPS) from GSE101905 (Knaupp et al., 2017).

PCA showed that bats were unique to all mammals, even the more distant ones like dogs, clustered together in the PCA plot, while bats formed a separate distinctive group (FIG. 5A) despite including other closely related laurasiatherian mammals. Further analysis of the gene signature that contributed the most to the bat-specific gene expression profile in the PCA analysis was performed. The “leading edge,” was extracted, corresponding to the top 5% of the genes that fortified the difference in principal component 1 (FIG. 5B) when comparing bat with mouse pluripotent stem cells, corresponding to 674 genes. The list covered genes belonging to a broad spectrum of transcription factors, kinases, metabolic and homeostatic enzymes. For instance, it included the HMG-CoA synthase HMGCS2, the apolipoprotein APOA1, the cyclin CCNT1, plasminogen PLG, the pluripotency factors OCT4 and Nanog, Tmprss2 which is required for SARS-CoV-2 entry in humans and the ubiquitin ligase NEDD4 among many other categories. Given the broad spectrum of categories it was analyzed if the leading-edge genes were enriched for any particular biological pathway in gene ontology analyses. The leading-edge genes were further enriched for developmental controllers, proteins targeting membranes, including the endoplasmatic reticulum, lipid and cholesterol biosynthesis, and fibrinogen production. However, the most prominent groups were viral gene expression, viral transcription, and many sets of genes activated or suppressed after viral infection (FIG. 5C).

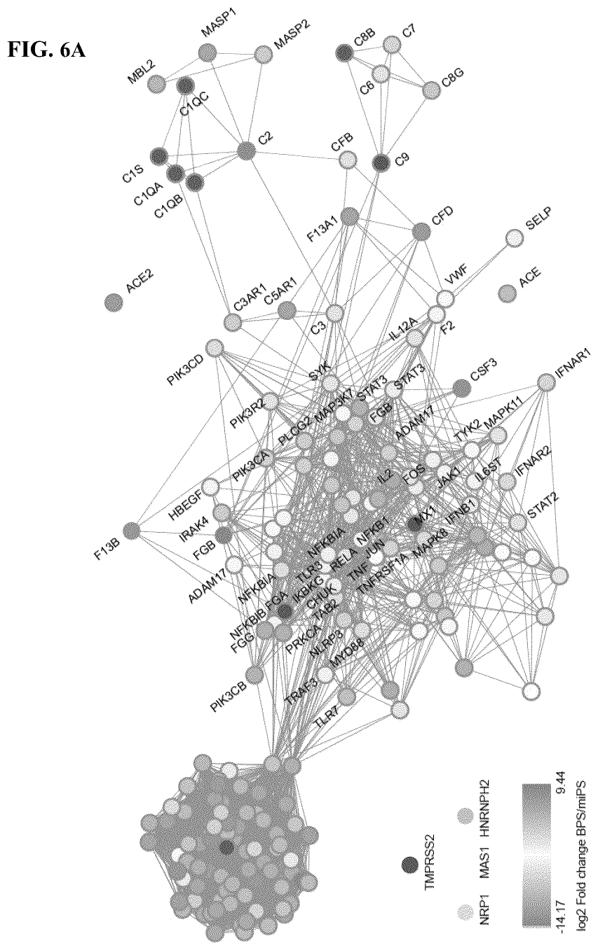

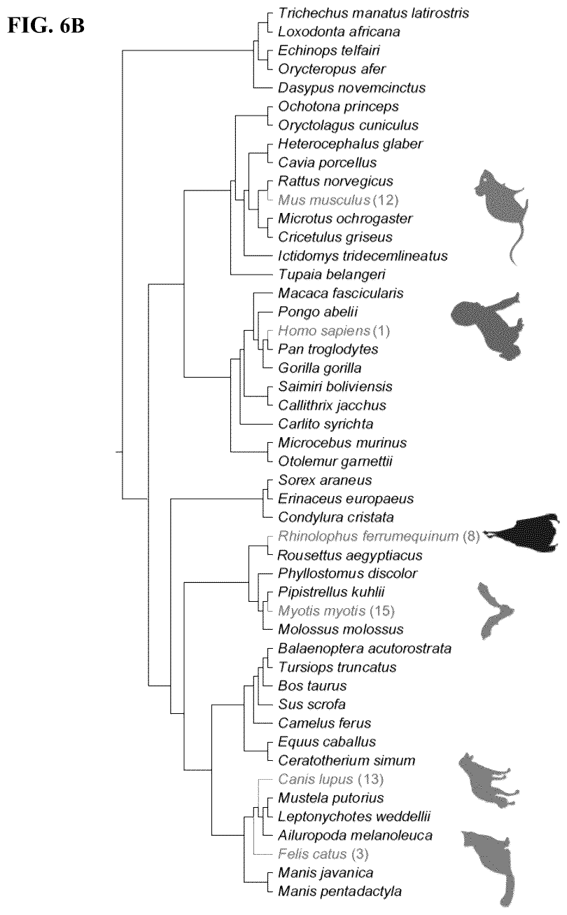

When analyzing the enrichment of any KEGG pathway, by far the most significantly enriched category was “Corona virus disease” (FIG. 5D, FIG. 6A). It almost seemed like bat stem cells executed a program normally activated after a virus infection. Interestingly, out of the set of leading-edge genes, only a total of eight genes showed significant evidence of positive selection in R. ferrumequinum, five of which showed at least one highly probable BEB site with no visual issues in the alignment region, while three genes (designated with ‘*’) did not (AARD, COL3A1, FAM111A, LAMB3, MUC1*, NES*, RGS5, RSPH1*) (FIG. 6B). Two of these genes, COL3A1 and MUC1, have roles in collagen formation in connective tissues protect against pathogen infections and showed evidence of selection in another bat species suggesting unique, bat-specific adaptations in these genes. The results might indicate that the unique bat signature is likely the consequence of the present viral sequences and that most of the coding leading edge genes are not under positive selection pressure.

Further, data were analyzed for the enrichment of transcription factor footprints in the mapping of open chromatin regions to these genes in the ATAC-seq data. Surprisingly, only two transcription factor motifs were significantly enriched, Klf5 and Ctcf Notably, however, these factors accompanied the majority of the genes in this set. Klf5 is a canonical pluripotency factor, which is essential for early embryogenesis and self-renewal of pluripotent stem cells. The recruitment of Klf5 binding sites to a new set of genes makes it likely that bat stem cells acquired novel features under the influence of this transcription factor. Ctcf, on the other hand, contributes to the establishment of higher-order genome structures (topologically associating domains), which are evolutionarily stable.

The leading-edge genes showed that they were under a purifying and positive selection. Of the 655 orthologous genes analyzed, a significant intensifying, purifying selection was observed in only five (Rsph1, Nes, Col3a1, Rgs5, and Lamb).

### MEME-ChIP

First, the ATAC-seq regions were identified that showed a shrunkelog2 fold change of 5 between bat fibroblast and pluripotent stem cells and an adjusted p value of less than 0.1 that were within 10 kb (i.e., any interval within 10 kb upstream or downstream) of any gene that is part of the top 5% of genes contributing to the differences in PC1 in the PCA analysis described above. The DNA sequences corresponding to these ATAC-seq regions were extracted from the GCF_004115265.1 reference genome und used in a MEME-ChIP motif search to identify sequence motifs (6-15 bp in width) for protein binding sites that are enriched in this set of genes (Machanick and Bailey, 2011; www.meme-suite.org/meme/tools/meme-chip). The sequence motifs with a p-value below 0.05 were then used in a FIMO analysis to identify the genomic positions and gene association of these motifs within the gene set. The number of genes associated with each motif within the gene set was then plotted against the factor known to bind to the and labeled with the protein know to bind to the motif

### Evolutionary Selection Analysis

To explore evidence of positive selection in R. ferrumequinum for the 674 genes identified as part of the “leading” edge in the PCA analysis described above, all gene alignments were extracted that were available for these transcripts (n=491) and had previously been annotated (Jebb et al., 2020), in addition to annotating 169 alignments that had been made available as part of BATIK but were currently unannotated. These alignments contained a maximum of 48 species from all eutherian mammalian superorders, with the species tree published by Jebb et al. (2020) used for all selection analyses. A total of 660 of these alignments contained representative genes for R. ferrumequinum and were analysed for positive selection using the branch-site models in the codeml package of the PAML suite of software (Yang, 2007). Positive selection was inferred using likelihood-derived dN/dS (o) values under both a null (foreground and background ω constrained to be less than 1) and alternative (foreground ω can vary) model. The R. ferrumequinum lineage was designated as foreground branch to detect unique instances of taxon-specific positive selection. A likelihood ratio test (LRT, 2*lnLₐₗₜ-lnLₙᵤₗₗ) was used to compare the fit of both models, with a p-value calculated assuming chi-squared distributed LRTs. P-values were corrected for multiple testing using the Benjamin-Hochberg False Discovery Rate (FDR) method via ‘padjust’ implemented in R. Any significant gene showing a p-value greater than 0.05 with ω>1 was explored further. Significant sites showing positive selection were identified using Bayes Empirical Bayes (BEB) scores with a probability >0.95. All significant genes were subject to a visual inspection of the alignment, to rule out potential false positive results having occurred due to misaligned sequences. In addition to R. ferrumequinum, the Myotis myotis (n=637 representative genes), Homo sapiens (n=652), Mus musculus (n=628), Canis lupus (n=593) and Felis catus (n=603) lineages were also independently designated as foreground branches for all genes containing a representative sequence shared with R. ferrumequinum. This served as a means of determining whether positive selection identified in R. ferrumequinum was truly unique to the species lineage or a consequence of bat-specific, Laurasiatherian-specific, or eutherian mammal-specific instances of sequence evolution.

### Gene Ontology and KEGG Pathway Analyses

Gene ontology and KEGG pathways that are enriched within a group of genes were identified with the Enrichr tool (Xie et al., 2021; www.maayanlab.cloud/Enrichr/). The odd ratios were then plotted with ggplot2 (Wickham, 2016; www.cran.r-project.org/web/packages/ggplot2/index.html) with the odds ratio displayed on the x-axis, the dot size reflecting the gene count (number of genes present in the top 5% of PC1 contributing genes) and the dot color reflecting the p-value.

### Protein Interaction Network in Bat IPSCs

In order to understand if the leading-edge genes that make horseshoe bats unique were enriched for any particular functional gene ontology category (FIG. 5C-D). The genes of the Corona virus disease related KEGG pathway were retrieved from the PathCards database (www.pathcards.genecards.org).

The differential expression analysis was performed between bat (this study) and mouse iPS cells (GEO accession number: GSM1287736, GSM1287747 and GSM1287748 from Study GSE53212 (Carter et al., 2014) using DESeq2 (Love et al., 2014). The Corona virus disease-related genes were then illustrated with Cytoscape (Version 3.8.2, Shannon et al., 2003) using the STRING protein query with a 0.8 confidence score cutoff. The nodes were colored based on the log 2FoldChange with a negative (blue) fold change indicating down-regulation and a positive (red) fold change indicating upregulation in bat pluripotent stem cells cells. Bold borders indicate proteins that were present in the top 5% of PC1 in the PCA analysis described above.

When analyzing the enrichment of any KEGG pathway, by far the most significantly enriched category was “Corona virus disease” (FIG. 5D, FIG. 6A). It almost seemed like bat stem cells executed a program normally activated after a virus infection. Interestingly, out of the set of leading-edge genes, only a total of eight genes showed significant evidence of positive selection in R. ferrumequinum, five of which showed at least one highly probable BEB site with no visual issues in the alignment region, while three genes (designated with ‘*’) did not (AARD, COL3A1, FAM111A, LAMB3, MUC1*, NES*, RGS5, RSPH1*) (FIG. 6B). Two of these genes, COL3A1 and MUC1, have roles in collagen formation in connective tissues protect against pathogen infections and showed evidence of selection in another bat species suggesting unique, bat-specific adaptations in these genes. The results might indicate that the unique bat signature is likely the consequence of the present viral sequences and that most of the coding leading edge genes are not under positive selection pressure.

### Example 7 Identification of Virus Like Structures in Bat IPSCs

This example describes the identification of virus like structures in bat IPSCs.

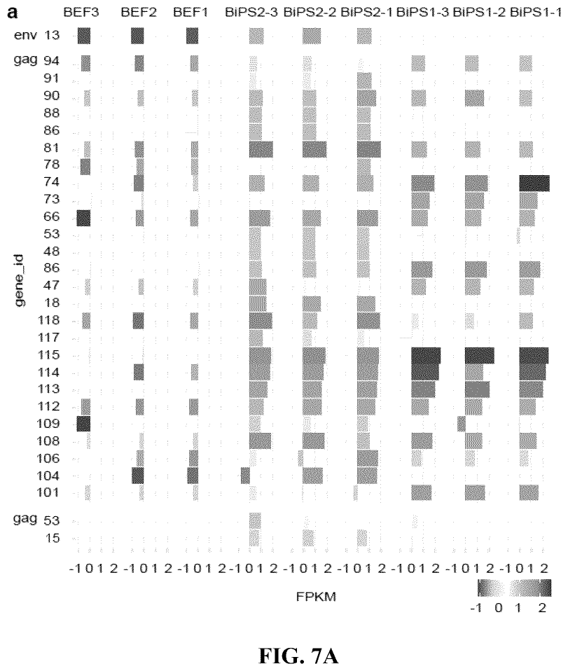

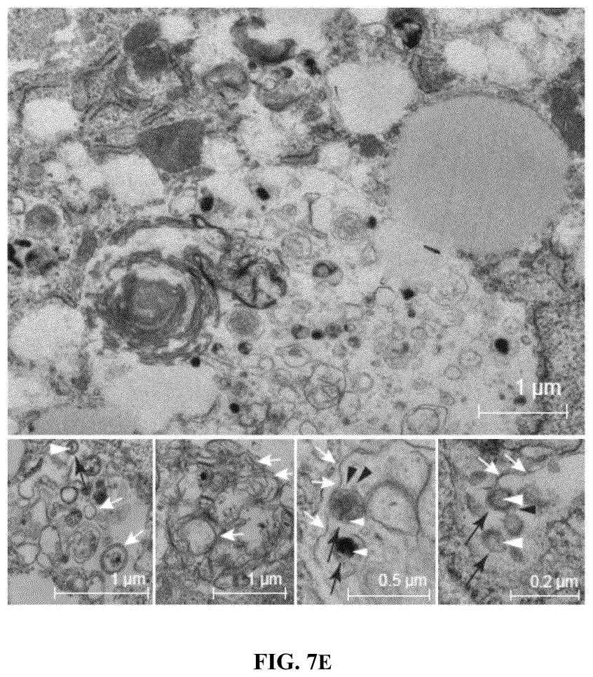

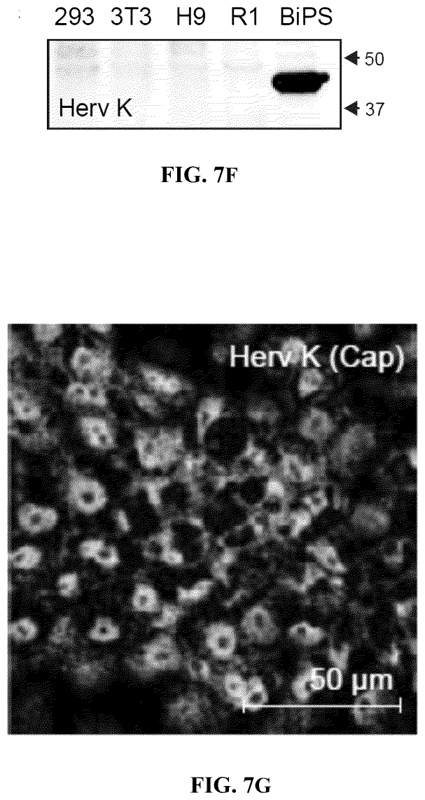

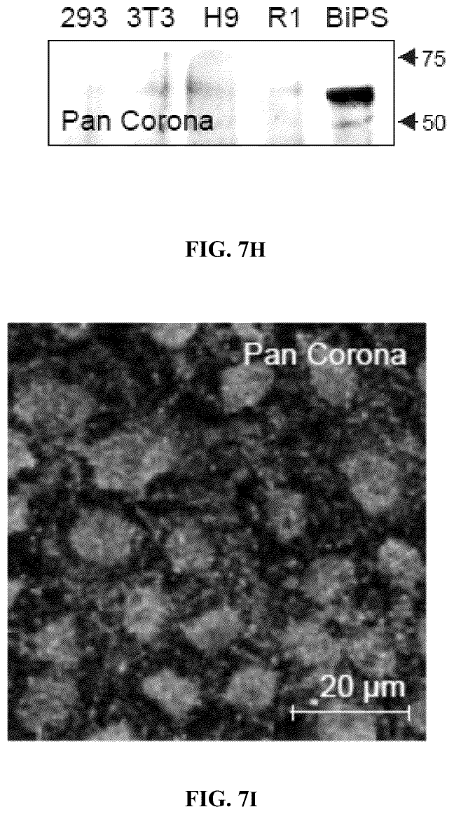

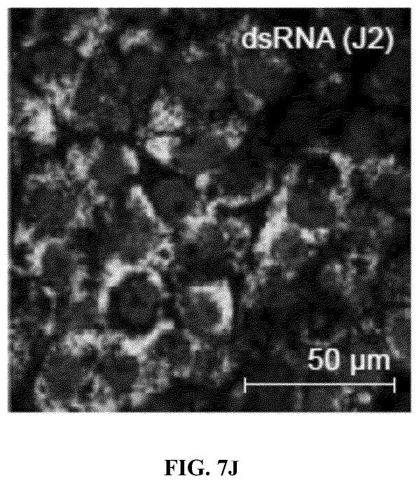

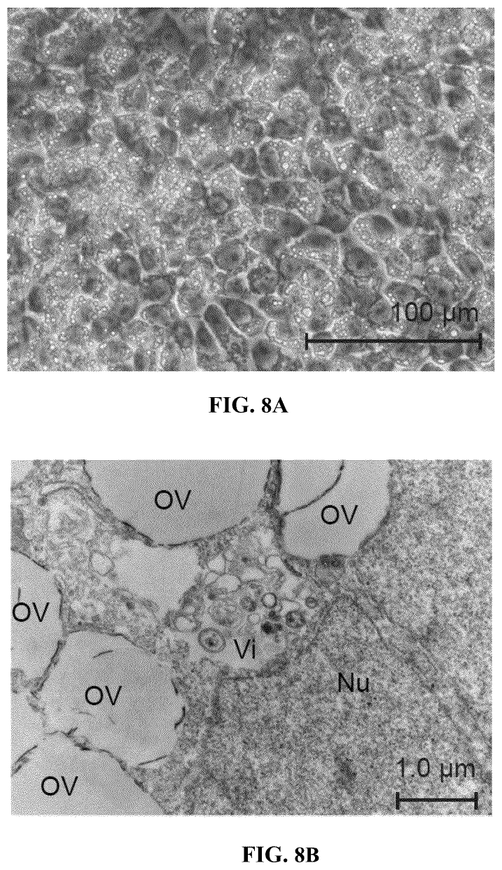

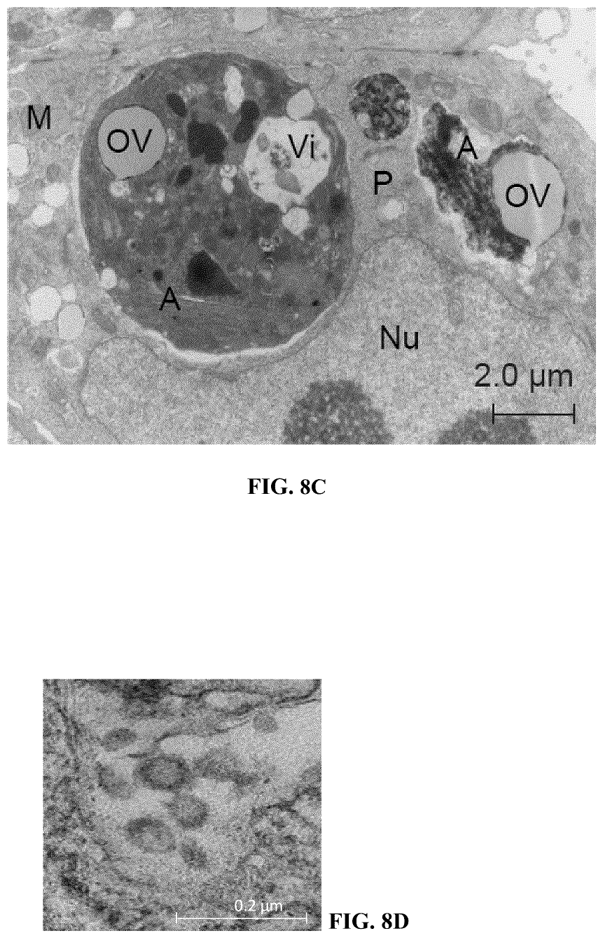

Briefly, bat IPSCs were imaged with differential interference contrast microscopy and Image-based flow cytometry. Images of the bat IPSCs highlighted prominent cytoplasmic vesicles. Bat stem cells were observed to be packed with small, luminescent vesicles that filled a significant proportion of the cytoplasm (FIG. 7A, FIG. 8A).

### Electron Microscopy and IMMUNOSTAINING

In order to analyze the vesicles, ultrastructural studies were performed using electron microscopy. Cells were grown in chambered Permanox slides (LabTek, MI) on irradiated mouse embryonic fibroblasts as described above for 5 days and then further processed by the Biorepository and Pathology core at the Icahn School of Medicine at Mount Sinai. Briefly, the cells were rinsed once with DPBS and fixed overnight with 2% paraformaldehyde and 2% glutaraldehyde in 0.01 M sodium cacodylate buffer at 4° C. Sections were rinsed in 0.1 M sodium cacodylate buffer, followed by a quick rinse with ddH₂O. Cells were post fixed with 1% aqueous osmium tetroxide for 1 hour, followed with an En bloc stain of 2% aqueous uranyl acetate for 1 hour. Sections were washed again in ddH₂O, dehydrated through graduated ethanol (25-100%), infiltrated through an ascending ethanol/epoxy resin mixture (Embed 812, EMS), and then covered with pure resin overnight. Chambers were separated from the slides, and a modified #3 BEEM embedding capsule (EMS) was placed over defined areas containing cells. Capsules were filled with pure resin and placed in vacuum oven to polymerize at 60° C. for 72 hours. Immediately after polymerization, the capsules were snapped from the substrate to dislodge the cells from the slide. Semithin sections (0.5-1 μm) were obtained using a Leica UC7 ultramicrotome (Leica, Buffalo Grove, IL), counterstained with 1% Toluidine Blue, cover slipped and viewed under a light microscope to identify successful dislodging of cells. Ultra-thin sections (85 nms) were collected on 300 hexagonal mesh copper grids (EMS) using a Coat-Quick adhesive pen (EMS). Sections were counter-stained with uranyl acetate and lead citrate and imaged with a Hitachi 7700 Electron Microscope (Hitachi High-Technologies) using an advantage CCD camera (Advanced Microscopy Techniques). Images were adjusted for brightness, contrast, and size using Adobe Photoshop CS4 11.0.1.

Data analysis showed that the vesicles were lipid or glycogen-filled vesicles and autophagosomes (FIG. 8B), all reported previously in bat inner cell mass cells and other pluripotent stem cells. The most prominent vesicles, some surrounded by lipid membranes, contained a significant number of structures resembling viruses-like particles (FIG. 7B).

Interestingly, the virion structures did not belong to a uniform set of virus categories. While some exhibited features of (endogenous) retroviruses, other virus-like particles were packed in highly electron-dense material and resembled DNA viruses. Finally, numerous intermediate assemblies were much smaller than the more “mature viruses” but could also be defective exogenous retroviruses and many of them were embedded in double-membrane structures (FIG. 7B). Some of the virus-like particles must have been shedding into the supernatant as significant levels of retroviral activity (1.21*10¹⁰ viral particles per mL) were detected in the culture medium. These observations suggest that bat cells either produce active particles through endogenized sequences in their genome or through persistent infection that was already present in the BEFs. Previously, ERV-like particles have been reported in naive pluripotent stem cells in mice and humans, and western blotting and immunostaining revealed high quantities of ERV antigen in the cytoplasm of bat stem cells (FIG. 7D, and FIG. 7F). Additionally, bat stem cells were positive for coronavirus antigen in western blots and immunostaining (FIG. 7C, and FIG. 7E) and stained positive with an antibody raised against double stranded RNA viruses (FIG. 7G), suggesting endogenous infection and expression of endogenized viruses or fragments of endogenized viruses on an unprecedented scale, not seen in other tumor or stem cell lines.

### Image-Based Flow Cytometry (ImageStream)

Cells were seeded onto 6-well plates and separated from irradiated MEFs via two-stage trypsinization after four days. Wells were dosed and incubated with 0.25 ml prewarmed (37° C.) trypsin which was removed and discarded at 4 minutes. An additional 0.25 ml trypsin was added and the plate was again incubated. After eight minutes cells were removed and pelleted via centrifugation. The cells were washed twice in PBS containing 0.5% BSA, fixed and permeabilized with Cytofix/Cytoperm. The Primary antibody was added at a dilution of 1:200 in wash buffer incubated overnight at 4° C. The cells were washed twice with 0.5% BSA/PBS, resuspended in wash buffer containing the secondary antibody at a 1:200 dilution Cells were then resuspended in wash buffer, the secondary goat anti-mouse AF568 antibody and incubated for 1 hour at 4° C. The cells were washed as before resuspended in 0.5% BSA/PBS containing two drops/ml DyeCycle Violet to stain the nuclei.

Imaging was conducted with the ImageStream MkII, at 60× magnification with the extended depth of field mode for probe resolution. Images were acquired using the INSPIRE 2.0 software at the lowest flow speed. Fluorophores were excited by the 405 nm and 568 nm lasers at 60 mW and 100 mW, respectively. Cells in focus were gated via histogram of brightfield gradient R. M.S. values and an aspect ratio vs. area plot was used to select the population of single cells. 5000 individual images of focused single cells were taken. Gating was refined further post-acquisition via the IDEAS 6.2 software suite by the same methods and plots, yielding n=1846 (BiPS). This software was used also for image processing, in which a set of custom masks defined by logical operators were used to denote vesicles and sensitively assess probes. For vesicles, it was observed that they may be selected from other cell component by contrast (bright and dark) and also by aspect ratio, and therefore are defined here by “Dilate(Range(Dilate(Range(System(Peak. (Threshold(M01, BF, 70), BF, Bright, 1), BF, 20), 0-5000, 0.4-1), 1), 0-5000, 0.4-1), 1) Or Range (AdaptiveErode(LevelSet(M01, BF, Dim, 5), BF, 75), 0-5000, 0.5-1).” BF and BF2 represent each brightfield image taken of a single cell from each of the two cameras, M01 and M09 represent the corresponding channel masks for each channel and the remaining terms represent mask modifiers and their associated values in the IDEAS software. For resolving immunofluorescence, “Peak(System(M05, Ch05, 3), Ch05, Bright, 1)” where Ch05 represents the staining of interest and M05 represents the corresponding channel mask. Modification was necessary to sensitively include all representative fluorescence, and to distinguish individual foci. The nuclear mask corresponding to DyeCycle Violet staining was defined “Object(M07, Ch07, Tight)” and the cytoplasm was defined through subtraction of the nuclear and vesicle masks from the cell mask through the logical operator available in the software (“Not”). Vesicle-nucleus overlap was determined in favor of vesicles by excluding them from the nuclear mask (“Not”). Probe localization was then defined according to these entities using the respective definitions and the operator “And.” Statistics for foci were generated using the Spot Count feature with a connectedness of 4. Prism 9 was used for graphs and statistics.

The results show that the bat stem cells were positive for coronavirus antigen in western blots and immunostaining (FIGS. 7H and I), and double-stranded RNA in immunostaining (FIG. 7J). The latter is considered a hallmark for the presence of replicative genomes from positive-strand and double stranded RNA viruses. Super-resolution imaging showed that the dsRNA was present in aggregates (micron-order) throughout the cytoplasm but essentially absent from the nucleus. Further, ImageStream analysis indicated a close quantitative relationship between viral antigens and the intracellular vesicles. Based on these findings, it appears that pieces of endogenous viruses are being expressed at a scale that has not been observed before in any other tumor or stem cell lines originating from other animals and humans.

### Example 8 Identification of Retroviral Sequences in the Bat Pluripotent Stem Cell

This example describes the identification of retroviral sequences in the bat IPSC.

### Retrovirus Assay

2 ml of tissue culture medium were collected, and retroviral particle concentrations were determined using the QuickTiter Retrovirus Quantitation Kit (Cell Biolabs) according to the manufacturer's instructions.

### Reverse Transcriptase Assay

Reverse transcriptase enzyme levels were determined with the colorimetric reverse transcriptase kit (Roche) per the manufacturer protocol. Cells lines represented were lysed in RIPA buffer, frozen at −80° C., thawed on ice, collected and resuspended in the kit lysis buffer (10 μL pellet in 40 μL lysis buffer per colorimetric well). Incubation duration (15 h at 37° C.) was selected for maximal sensitivity to the limit of the kit (1-5 pg RT). Absorbance at 405 nm was measured by microtiter ELISA plate reader. Sample absorbance measurements were fitted to a linear regression of the measured HIV-1 RT standards (Y=2.549×) to obtain RT concentrations in units of ng/well. The results show, that some of the virus-like particles shed from the BiPS into the supernatant as substantial levels of viral particles (1.21*1010 viral particles per mL as determined in a retroviral assay and 0.3 ng/well in a direct reverse transcriptase assay) were detected in the culture medium.

### Plaque Assay

Supernatants were centrifuged at 10000 rpm for 5 min to remove cellular debris, and the cleared lysates transferred to new tubes. Lysates were then diluted in 10-fold dilutions 6 times. Quantification of infectious titer was then performed by plaque assays in comparison to SARS-CoV-2 infection as positive control. Briefly, Vero-E6 cells were plated as confluent monolayers in 12 well dishes. Media was removed, and wells washed in 1 ml of PBS. 200 ul of diluted lysates was then added per well and allowed to incubate for 1 hour at 37° C. After viral adsorption, lysates were removed from the well and cells were overlaid with Minimum Essential Media supplemented with 2% FBS, 4 mM L-glutamine, 0.2% BSA, 10 mM HEPES and 0.12% NaHCO3 and 0.7% agar. 72 h post infection, agar plugs were fixed in 10% formalin for 24 h before being removed. Plaques were visualized by staining with TrueBlue substrate (KPL-Seracare) and viral titers calculated and expressed as PFU/ml. Immunostaining with an antibody detected the endogenous retrovirus protein Herv K or a Pan Corona antibody in Rhinolophus ferrumequinum embryonic fibroblasts. Immunostaining with a Pan corona antibody in Myotis myotis fibroblasts or induced pluripotent stem cells (iPS) is shown in FIG. The results show that inoculated Vero cells with cell culture supernatant of the bat iPSCs in the plaque assay did not detect any measurable cytotoxic effects in contrast to acute infectious virus particles that served as positive controls (SARS-CoV-2 particles).

### Metapneumovirus (MPV) Infection of BiPS and mES Cells

50,000 mouse ES cells (R1) or BiPS cells were plated per well of a 12-well plate on irradiated CF1 mouse embryonic fibroblasts using mouse and bat culture medium respectively. After 24 hours, culture medium containing human Metapneumovirus with GFP (MPV-GFP) (ViralTree) with a final multiplicity of infection (MOI) of 3. Medium was changed daily, and samples were dissociated at 3 and 5 dpi using trypsin/EDTA and the infection rate was determined by fluorescence activated cell sorting (FACS).

In line with the pro-viral environment that was observed transcriptionally, bat stem cells infected with an exogenous Metapneumovirus (MPV) in comparison with mouse stem cells revealed a particularly permissive environment for viral persistence, further underscoring the supportive nature of bat stem cells for viruses. These results suggest that bat stem cells execute a program that in other mammalian cells is activated only after a virus infection.

### Example 9 Identification of Viral Sequences in the Bat Pluripotent Stem Cell Transcriptome

This example describes the identification of viral sequences in the bat IPSC transcriptome.

Endogenization of an unusually varied group of viral genomes has occurred in bats (for example described in Banerjee et al. 2020; Katzourakis and Gifford 2010; Jebb et al. 2020). Endogenized viral sequences are reactivated and tolerated by all pluripotent stem cells (Grow et al. 2015). As a result, bat pluripotent stem cells should express and tolerate a particularly wide range of endogenized viral sequences. First, endogenous retroviruses, which are abundant and diverse in bat genomes (Jebb et al. 2020; Hayward et al. 2013; Skirmuntt and Katzourakis et al. 2019) were analyzed. As a starting point, anchor points of retroviral sequences that had been previously mapped (Jebb et al. 2020) were picked. To obtain a broader portrait of the virus-like particles and approximate their identity more specifically, RNA-seq data was re-analyzed and additional long-read RNA sequencing (iso-seq) was performed.

### Iso-Seq Library Preparation and Sequencing

Cells were lyzed in 400 μl Trizol reagent (Life Technologies) and total RNA was extracted using the AllPrep DNA/RNA Mini Kit (Qiagen) including a DNase digest to remove any potential contamination from carryover of genomic DNA using RNase-free DNase (Qiagen,) according to the manufacturer's instructions. The extracted RNA was then purified using 1.8×RNAClean XP beads (Beckman Coulter) to remove any molecular impurities. Iso-Seq SMRTbell libraries were prepared as recommended by the manufacturer (Pacific Biosciences). Briefly, 300 nanograms of total RNA (RIN>8) from each sample was used as input for cDNA synthesis using the NEBNext Single Cell/Low Input cDNA Synthesis & Amplification Module (NEB,), which employs a modified oligodT primer and template switching technology to reverse-transcribe full-length polyadenylated transcripts. Following double-stranded cDNA amplification and purification, the full-length cDNA was used as input into SMRTbell library preparation, using SMRTbell Express Template Preparation Kit v2.0. Briefly, a minimum of 100 ng of cDNA from each sample were treated with a DNA Damage Repair enzyme mix to repair nicked DNA, followed by an End Repair and A-tailing reaction to repair blunt ends and polyadenylate each template. Next, overhang SMRTbell adapters were ligated onto each template and purified using 0.6×AMPure PB beads to remove small fragments and excess reagents (Pacific Biosciences). The completed SMRTbell libraries were further treated with the SMRTbell Enzyme Clean Up Kit to remove unligated templates. The final libraries were then annealed to sequencing primer v4 and bound to sequencing polymerase 3.0 before being sequenced on one SMRTcell 8M on the Sequel II system with a 24-hour movie each. After data collection, the raw sequencing subreads were imported to the SMRTLink analysis suite, version 10.1 for processing. Intramolecular error correcting was performed using the circular consensus sequencing (CCS) algorithm to produce highly accurate (>Q10) CCS reads, each requiring a minimum of 3 polymerase passes. The polished CCS reads were then passed to the lima tool to remove Iso-Seq and template-switching oligo sequences and orient the isoforms into the correct 5′ to 3′ direction. The refine tool was then used to remove polyA tails and concatemers from the full-length reads to generate final full-length, non-chimeric (FLNC) isoforms. The FLNC isoforms were then clustered together using the cluster tool to generate final, polished consensus isoforms per sample.

Briefly, the existence of viruses in the Rhinolophus ferrumequinum transcriptome was explored by analyzing the RNA-seq and Iso-seq data based on a metagenomic approach using Kraken2 v2.1.2 (Wood et al, 2019). First, the adaptors in the RNA-seq data were removed with Trimgalore v0.6.7 (Krueger et al., 2021) and all replicates for corresponding datasets were joined in one file. The reference library “RefSeq complete viral genomes/proteins” was downloaded and a custom database was built to identify matches within the processed RNA-seq or Iso-seq. To eliminate false positive hits that could be due to matches with any cellular transcript such as oncogenes that are carried by some viruses, a second analysis was performed after eliminating all reads from the RNA-seq and Iso-seq datasets that matched any annotated Rhinolophus ferrumequinum transcript. To do this, the Iso-Seq FLNC isoforms or RNA-seq trimmed fastq sequences were first mapped to the “Rhinolophus ferrumequinum genomic ma exons RefSeq” file “GCF_004115265.1_mRhiFer1_v1.p_rna_from_genomic.fna” using gmap/gsnap (doi.org/10.1093/bioinformatics/bti310). The sequences with no mappings were then used to identify viral sequences using Kraken2 as before.

### Mapping of RNA-Seq Reads to Bat Genomes and Quantifying Expression of ERVs

To trim adapters and generate quality metrics of the fastq files, Trimmgalore v.0.6.6 (www.github.com/FelixKrueger/TrimGalore), a wrapper for Cutadapt (www.github.com/marcelm/cutadapt) and FastQC (www.bioinformatics.babraham.ac.uk/projects/fastqc/) were used. Then, reads were mapped to the genome of R. ferrumequinum (Bat1K assembly HLrhiFer5) using HISAT2 v.2.2.1 (PMID: 31375807) suppressing unpaired alignments for paired reads (--no-mixed), suppressing discordant alignments for paired reads (--no-discordant), and setting a function for the maximum number of ambiguous characters per read (--n-ceil L,0,0.05). Output files were then filtered to remove any unmapped reads (-F 4), sorted and indexed using samtools (PMC2723002). Aligned reads were then assembled into transcripts using stringTie v2.2.1 (PMC4643835) in stranded mode (-rf). To generate a Ballgown readable expression output with normalized expression units of fragments per kilobase of transcript per million mapped fragments (FPKMs), the Bat1K annotation of known endogenous retrovirus (ERVs) for R. ferrumequinum (PMID: 32699395) (www.genome.senckenberg.de/) were also used as input in strigTie. Output counts were post-process and plotted with a custom R script.

### Mapping of Iso-Seq Reads to Bat Genomes and Identifying ERVs

Iso-Seq transcripts were mapped to the genome of R. ferrumequinum (Bat1K assembly HLrhiFer5) using minimap2 (PMC6137996) in mode for long-read/Pacbio-CCS spliced alignment (-ax splice:hq), giving priority to known splice sites from an input annotation (BatIK), to find canonical splicing sites GT-AG in the transcript strand (--junc-bed -uf), with a cost of 5 for a non-canonical GT-AG splicing (-C5), and excluding from the output any secondary alignments (--secondary=no). Output files were then filtered to remove any unmapped reads or those not aligned to the primary alignment (-F 260), sorted and indexed using samtools (PMC2723002). Aligned transcripts to the genome were intersected with known ERVs.

### De Novo Assembly of Potential Virus-Derived RNA-Seq

The trimmed reads that were identified by Kraken2 v2.1.2 to map to viral sequences with a confidence score of 0 as described above were classified as either mammalian or non-mammalian using the VIRION database (Carlson et al., 2022) based on their viral taxonomic ID assigned by Kraken2. The data were converted to FASTA format using the Seqtk v1.3 program and the reads were assembled using the Trinity v2.12 software. To check and gather successful assemblies that had produced at least one contig, a custom BASH script was applied for both groups of mammalian and non-mammalian viruses.

### Mapping Transcripts to Viral and Mammal Databases

To determine if the assembled transcripts represented an expressed viral sequence, all transcripts were mapped to a database of viral genomes using BLAST. The viral database consisted of genomes whose host species contained either ‘human’ or ‘vertebrate’ as specified in the NCBI database. Initially this list contained over 17,000 genomes. However, this was reduced to 3,922 genomes by taking only unique virus/strain names. An additional non-mammalian virus database was generated by combining all genomic sequences of viruses identified by Kraken2 and classified as non-mammalian via VIRION.

Transcripts were also mapped to a combined database of bat, human and mouse genomes to both confirm their presence in the bat and to exclude the possibility of false positives through contamination. For each of these transcripts, expected values for both bat and viral genome BLAST results were combined into a single metric via the following formula: Log (bat-expected value+1×virus-expected value+1). A threshold of less than 0.3, representing a combined e-value of less than 1e⁻⁵⁰ for both viral and bat hits, was used to rule out potential false positives. In addition, SQUID (www.eddylab.org/software.html) was used to shuffle the 63 (bottom-up) and 82 (top-down) sequences while preserving the dinucleotide distribution (parameter -d) to obtain a conservative threshold to distinguish bona fide viral homology from matches by random chance. Shuffled sequences were mapped to both the bat genome and viral genome databases, with the same BLAST threshold applied. All transcripts passing this threshold were extended by 5000 bp flanks within the bat genome and these regions were subsequently mapped to the viral database to confirm their presence in a viral genome.

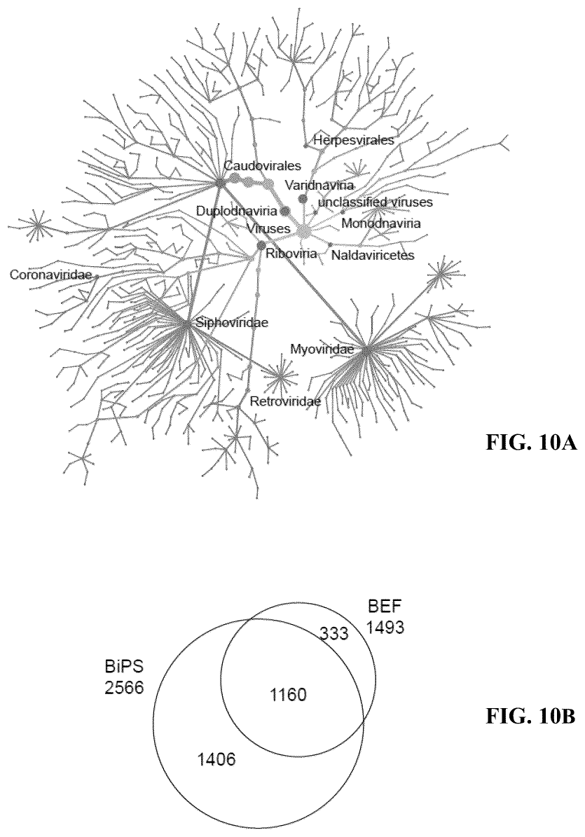

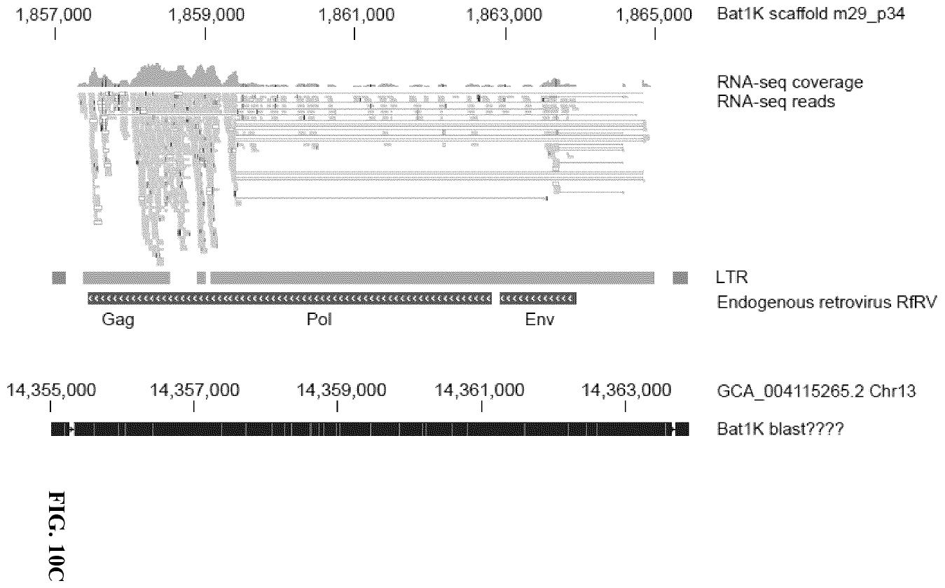

The resulting sequencing reads were mapped against a virus database, using a metagenomic classification tool (Kraken). Mapping of the RNA-seq data revealed the expression of a widely diverse set of retroviral families in bat pluripotent stem cells, which was undetectable in BEFs. The results revealed a taxonomically highly diverse “zoo” of assigned viruses belonging to several significant viral families (FIG. 9A-C, FIG. 10A). They included, but were not limited to, Paramyxoviridae, Rhabdoviridae, Filoviridae, Bornaviridae, Flaviviridae, Coronaviridae, Picomaviridae, and Retroviridae (FIG. 9A-C, FIG. 10A). Similarly, viral sequences in BEFs were analyzed, notably yielding some viral sequences but to a much lesser degree (FIG. 10B). This finding is surprising as post-implantation tissues typically do not exhibit endogenous viral activity, underscoring pro-viral environments that bats create. Hence, the metagenomic analysis strongly suggests the remarkable possibility that bat stem cells harbor a significant number of viral-like sequences.

The potential for confounding effects that might impact the metagenomic assessment could be three potential sources for distortions: (i) statistical stringency, (ii) cellular genes containing viral-like sequences (e.g., oncogenes), and (iii) potential xeno sequence pollution originating from the feeder cells. To address the first point, progressively higher statistical stringency was used, yielding an expected decrease in matches. However, even under the most binding conditions, it still resulted in a sizable number of hits. To exclude potential cellular genes misinterpreted by the classification algorithm as viruses, the RNA-seq and iso-seq were depleted from all sequences that match exons, which only marginally affected the number of hits. Finally, some of the classified sequences were checked for murine origin as was the case for several retroviruses. Somatic tissue-derived cells, such as mouse fibroblasts, do not express endogenous viruses in measurable quantities. Hence, the ability to readily detect such sequences may suggest the intriguing possibility that the BiPS cells triggered their activation and expansion or even the infection of the BiPS cells. While confounding effects could affect the metagenomic classification process, it is highly likely that a significant body of proviral sequences inhabits BiPS cells.

### Example 10 Assembly of Novel Viral Sequences

This example describes the assembly of novel full-length viruses, shorter viral insertions, and novel, more distant viruses based on the sequencing data from BiPS cells.

As a starting point, anchor points of retroviral sequences that had been previously mapped were picked. Curation of the RNA sequences predicted to match those genomic sequences allowed the identification of not only previously described full-length bat retroviruses (RFeRV, FIG. 10C) but also an undiscovered full-length retrovirus sequence, RFe-V-MD1 (FIG. 9D, SEQ ID NO:1). The RNA sequencing also readily revealed short integrated viral sequences, for instance, Columbid/Falconid herpesvirus and Sindbis virus (FIG. 9E, FIG. 10A). In this case, the metagenomic classification tool pointed to this sequence. Upon closer inspection, it was found that the transcripts came from a genomic region immediately adjacent to a LINE-1 sequence. Furthermore, it was discovered that some of the sequences formed stem-loop structures, thus suggesting a potential functional role of the RNA (FIG. 9F). Another case at point was a region residing in the first intron of the XPA gene (a DNA damage and repair factor) on chromosome 12. A BLAST search with the fragment showed homology to two human herpesvirus 4 isolates (HKD40 and HKNPC60), the human respiratory syncytial virus (Kilifi isolate), and a fragment of about 500 bp that was identified at the end of a SARS-CoV2 isolate in an infected patient (FIG. 11C, FIG. 9G). Additionally, a protein translation search discovered homologies to an RNA-dependent DNA polymerase of the lymphocystis disease virus and the erythrocytic necrosis virus (FIG. 11B). Finally, expression data in conjunction with the bat genome was analyzed for more distant viral sequences using metagenomic classification taxonomies. Analysis for spike protein-like sequences found distant matches, a nearly 50% identical sequence to either RaTG13 (TABLE 4) or the Scotophilus bat coronavirus 512 (TABLE 3) covering most of the spike encoding sequences (FIG. 9H,). A phylogenic analysis revealed that these genomic sequences mostly resembled the spike protein-encoding genomic portion of human coronavirus 229E and the human coronavirus OC43, respectively (FIG. 11D). In both cases, a flanking LINE-1 sequence was present. This suggests that potential LINE elements are directly involved in the homing of viral RNA.

TABLE 2

| Identifier | Fragment/Read ID | Source | Size | | Identified | Homology | Summary of result |
|---|---|---|---|---|---|---|---|
| RFe-V-MD1 | m64019_210624_{—} | Iso-seq | 6088 | bp | Overlap of | Full length endogenous | Iso-seq sequence overlapping |
| | 011637/39584940/ccs | RNA | | | Iso-seq | retrovirus | with a predicted retroviral gag |
| | | | | | sequence | | sequence allowed for |
| | | | | | with | | identification of a novel full |
| | | | | | previously | | retroviral sequence. |
| | | | | | predicted | | |
| | | | | | gag | | |
| | | | | | sequence of | | |
| | | | | | an | | |
| | | | | | endogenous | | |
| | | | | | retrovirus | | |
| RFe-V-MD2 | m64019_210624_{—} | Iso-Seq; | 3350 | bp | Kraken | Columbid alphaherpesvirus | Kraken analysis of Iso-seq |
| | 011637/330171/ccs | | | | analysis of | 1; Tax ID: 93386 | reads identified homology |
| | kraken: taxid\|93386 | | | | Iso-seq data | | with Columbid alphaherpesvirus |
| | | | | | and | | 1. A subsequent Blast search |
| | | | | | sequence | | confirmed a partial alignment |
| | | | | | alignments | | with the Columbid and |
| | | | | | | | Falconid herpesvirus 1 as well |
| | | | | | | | as the Sindbis virus. The |
| | | | | | | | homologous sequence codes |
| | | | | | | | for a 24 aa strech that has 79% |
| | | | | | | | homology with hypothetical |
| | | | | | | | proteins |
| | | | | | | | CoHVHLJ_080/FaH\HV1S18_80 |
| | | | | | | | of the Columbid or Falconid |
| | | | | | | | herpesvirus, respectively. Part |
| | | | | | | | of the sequence that shows |
| | | | | | | | homology to the Sindbis virus |
| | | | | | | | defective interfering particle |
| | | | | | | | di-2 which has been shown to |
| | | | | | | | inhibit viral replication in |
| | | | | | | | infected cells in vitro (Monroe |
| | | | | | | | S S, Schlesinger S. RNAs from |
| | | | | | | | two independently isolated |
| | | | | | | | defective interfering particles |
| | | | | | | | of Sindbis virus contain a |
| | | | | | | | cellular tRNA sequence at |
| | | | | | | | their 5′ ends. Proc Natl Acad |
| | | | | | | | Sci USA. 1983 |
| | | | | | | | June; 80(11): 3279-83. doi: |
| | | | | | | | 10.1073/pnas.80.11.3279. |
| | | | | | | | PMID: 6304704; PMCID: |
| | | | | | | | PMC394024) and can form a |
| | | | | | | | hairpin structure. |
| RFe-V-MD3 | m64019_210624_{—} | Iso-Seq | 7955 | bp | Kraken | Ranid herpesvirus 1, | Kraken analysis of Iso-seq |
| | 011637/ | | | | analysis of | Tax ID: 85655 | reads identified reads that |
| | 128451663/ccs | | | | Iso-seq data | | show homology with the |
| | kraken: taxid\|85655 | | | | and sequence | | Ranid herpesvirus 1. |
| | | | | | alignments | | Alignment analysis revealed |
| | | | | | | | that the particular Iso-seq read |
| | | | | | | | matches a genomic DNA |
| | | | | | | | fragment in the first intron of |
| | | | | | | | the Rhinolophus |
| | | | | | | | ferrumequinum XPA gene (a |
| | | | | | | | DNA damage and repair |
| | | | | | | | factor) on chromosome 12 that |
| | | | | | | | is known to harbor a predicted |
| | | | | | | | LINE-1 sequence. Closer |
| | | | | | | | inspection of this Iso-seq read |
| | | | | | | | revealed homology with two |
| | | | | | | | Human herpesvirus 4 isolates |
| | | | | | | | (HKD40 and HKNPC60), the |
| | | | | | | | Human respiratory syncytial |
| | | | | | | | virus (Kilifi isolate) and an |
| | | | | | | | about 500 bp DNA fragment |
| | | | | | | | that was identified at the end |
| | | | | | | | of a SARS-CoV2 isolate from |
| | | | | | | | an infected patient. |
| | | | | | | | Additionally, a BlastX search |
| | | | | | | | discovered homologies to an |
| | | | | | | | RNA-dependent DNA polymerase of |
| | | | | | | | the Lymphocystis disease virus |
| | | | | | | | and the Erythrocytic necrosis |
| | | | | | | | virus. |
| RFe-V-MD4 | m64019_210618_{—} | Bat | 6404 | bp | Kraken | Scotophilus bat coronavirus | Genomic sequence found that |
| | 193151/ | genome | | | analysis of | 512; Tax ID: 693999 NCBI | has 42% Identity and 42% |
| | 159712964/ccs | | | | genomic | Reference: NC_009657.1 | Similarity with the |
| | kraken: taxid\|693999 | | | | reads | | Scotophilus bat coronavirus 512. |
| RFe-V-MD5 | hub_1489433_GCA_{—} | Bat | 4860 | bp | Target | Bat coronavirus RaTG13 | Genomic sequence found that |
| | 004115265.2_dna | genome | | | analysis of | Tax ID: 2709072: NCBI | shows 44% identity and 44% |
| | range = chr1: | | | | RFe genome | Reference: MN996532.2 | similarity with RaTG13 |
| | 38151239-38156098 | | | | with spike | | coronavirus. |
| | | | | | protein | | |
| | | | | | coding | | |
| | | | | | sequence of | | |
| | | | | | bat RaTG13 | | |
| | | | | | coronavirus | | |
| RfRV | Bat1k: scaffold_{—} | Cui J, et | 9649 | bp | Transcription | Previously identified | Transcription profile in RNA- |
| | m29_p_34: 1,856,366- | al., J | | | profile in | endogenous retrovirus | seq in genomic region that |
| | 1,866,014/GCA_{—} | Virol. 2012 | | | RNA-seq in | | overlaps with the previously |
| | 004115265.2: chr13: | April; 86(8): | | | genomic | | identified endogenous |
| | 14,355,027-14,363,924 | 4288-93. | | | region that | | retrovirus |
| | | | | | overlaps | | |
| | | | | | with the | | |
| | | | | | previously | | |
| | | | | | identified | | |
| | | | | | endogenous | | |
| | | | | | retrovirus | | |

TABLE 3
Alignment of identified sequence with the Scotophilus bat coronavirus 512
genomic sequence.

| Sequence 1 | | NC_009657.1 (SEQ ID NO: 352) | |
|---|---|---|---|
| Sequence 2 | | m64019_210618_193151_159712964_ccs (SEQ ID NO: 353) | |
| Matrix | | EBLOSUM62 | |
| Gap penalty | | 16 | |
| Extend penalty | | 4 | |
| Length | | 6654 | |
| Identity | | 2802/6654 (42.1%) | |
| Similarity | | 2802/6654 (42.1%) | |
| Gaps | | 383/6654 (5.8%) | |
| Score | | 10094 | |
| NC_009657.1 | 21507 | CAATTGCTTGGTTGCATTGCCTAAGTTG--CAAG-GTCTTACTACCACTC | 21553 |
| | | \|.\|.\|\|\|....\|\|.\|...\|\|...\|\|\|\|\| \|..\| \|.\|\|.\|\|...\|\|\|\|. | |
| m64019_210618 | 1 | CTACTGCAGTATTTCTCAGCTAGAGTTGTGCTGGCGACTCACAGTCACTT | 50 |
| NC_009657.1 | 21554 | -TGTCTTTTGACTCACCACTTAATGTGCCTGGGTT--TTCCTGTAACGGC | 21600 |
| | | .\|....\|\|.\|\|..\|\|\| \|\|.\|...\|\|\|\|\|\|..\| \|.\|\|\|\|.\|..... | |
| m64019_210618 | 51 | GAGGAACTTTACAAACC--TTTACAGGCCTGGACTCCTCCCTGAAGGTTT | 98 |
| NC_009657.1 | 21601 | GCCAATGGTTCTAGCTCAGCGGAAGCCTT-TCGTTTTAACGTCAATGATA | 21649 |
| | | ...\|.\|\|..\|\|.\|.\|\|.\|...\| \|\|\|\|. .\|.\|\|.\|....\|.\|...\|\|\| | |
| m64019_210618 | 99 | TTGACTGAGTCAATCTAATAAG--GCCTGGACATTGTGTATTTAGAAATA | 146 |
| NC_009657.1 | 21650 | CTAAGTTGT-TTGTTGGTGCTGGCGCTGTTACATT-GAACACCGTCGATG | 21697 |
| | | .\|.....\|. \|\|..\|\|.\|.\|...\|..\|\|\|\|.\|\|.. \|.\|\|....\|..\|\|. | |
| m64019_210618 | 147 | GTTCCCCGAGTTTCTGATACAACCCTTGTTTCAAAAGTACTGAATGTATA | 196 |
| NC_009657.1 | 21698 | GTGTTAATGTTTCTATTGTGTGCTCCAATAATGCAACACAGCCCACTAGG | 21747 |
| | | ....\|\|\|\|\|\|.\|\|..\|\|....\|.\|...\| \|\|.\|\|...\|.\|..\|\|.\|\|\|\|. | |
| m64019_210618 | 197 | AGTGTAATGTATCACTTCACAGATTTCA-AAAGCGTAAGAAACCTCTAGA | 245 |
| NC 009657.1 | 21748 | TCAA--ACAACTTGCAGGAAGACCTGCCTTACTATTGCTTCACTAACACT | 21795 |
| | | ..\|. \|\|\|..\|.\|...\|..\|..\|\|..\|\|\|..\|.\|..\|\|.\|..\|...\|\|. | |
| m64019 210618 | 246 | AAAGGTACAGTTAGTGAGGGGTACTTACTTCATCTCTCTCCTTTGCAACA | 295 |
| NC_009657.1 | 21796 | AGTAGCGGCACTAATCACACTGTTAAGTTTCTTTCAGTTTTCCCGCCAAT | 21845 |
| | | .\|....\|\|\| \|.\|\|. \|\|....\|\|.\|.\|..\|\|\|\|...\|.\|\|..\|\|. | |
| m64019_210618 | 296 | CGCTTAGGC-----TGACT-TGAACTGTCTTTACCAGTGGGCTCGAGAAG | 339 |
| NC_009657.1 | 21846 | CATTCGTGAGTTTGTGATCACCAAATATGGCAATGTCTATGTTAATGGCT | 21895 |
| | | .....\|.\|\|..\|..\|...\|...\|..\|...\|...\|....\|.\|\|..\|. \|\| | |
| m64019_210618 | 340 | TGAAAGGGATATCATTGGCGGTATCTGCTGGCCTACAGAAGTACAC--CT | 387 |
| NC_009657.1 | 21896 | ATATCTATTTGAGAACTAGACCATTGACAGCCGTGCACTTGAACGCATCC | 21945 |
| | | .\|.\|\|..\|\|\| ..\|\|\|\| \|\|\|.\|.\|.\|\|\|\|.\|..\|.\|\|... | |
| m64019_210618 | 388 | GTTTCCTTTT--------TGCCAT---CAGTCATTCACTGGCGCTCAGAG | 426 |
| NC_009657.1 | 21946 | TCTCATTCGCAGGACGTAGCAGGGTTTTGGACTATTGCCGCCACAAACTT | 21995 |
| | | .\|\|\|.\|\|.\|......\|..\|..\|\|\|\|..\|..\|\|\|\|.\|.. \|....\|..\|.. | |
| m64019_210618 | 427 | ACTCTTTAGACATTTGCTGATGGGTAGTCTACTAATAA-GTAGAATCCGA | 475 |
| NC_009657.1 | 21996 | CACGGATGTGCTTGTTGAGGTGAACAACACAGG-CATTCAGAGGTTGTTG | 22044 |
| | | \|.\| .\|\|......\|\|\|.\|...\|\|.\|.\|.\|. \|\|..\|..\|\|\|\|.\|\|\|. | |
| m64019_210618 | 476 | CCC----TTGAAGAAAGAGTTTTGCATCTCTGTTCACGCTCAGGTCGTTA | 521 |
| NC_009657.1 | 22045 | TATTGTGACACGCCTGAAAACAGTGTCAAATGTTCACAACTCTCTTTTGA | 22094 |
| | | .\|\|....\|.\|.\|..\|...\|.\|\|\| \|\|....\|\|..\|..\|.....\|..\|\| | |
| m64019_210618 | 522 | GATCAATAAATGTTTACCACCAG---CATGCTTTTCCTGCAGCAGTAAGA | 568 |
| NC_009657.1 | 22095 | ACTGGAGGACGGGTTTTATTCCATGACTGCAGATAATGTTTATGCAGTAA | 22144 |
| | | .\|...\|.\|\| .\|\|\|\|.\|.. \|\|.\|.\|.\|.\| .\|\|\|..\|\|. | |
| m64019_210618 | 569 | AATCATGAAC-------CTTCCTTTT----AGTTGAAGCT-GTGCGATAG | 606 |
| NC_009657.1 | 22145 | CTAAGCCCCACACGTTTGTGACTTTGCCCACGTTTAATGACCATGGGTTC | 22194 |
| | | .....\|.\|.\|.\|.\|\|.\|\|\|....\|\| \|\|.\|\|. .\|\|.\|\|\| \| | |
| m64019_210618 | 607 | ACTGTCTCAATATGTCTGTTTAATT-CCTACA-------GCCCTGG---C | 645 |
| NC_009657.1 | 22195 | GTTAATGTTACTGTGGGTGGTAACTTTGACAGTTCATACCCACCAAAGTT | 22244 |
| | | .\|.\|.....\|..\|\|.\|\|\|...\|\|\|.\|..\|.\|..\|\|\|....\| \|...\|\|\|\| | |
| m64019_210618 | 646 | ATAATCAGGAGGGTAGGTTTAAACATCAAAACATCAAGAAC-CTGGAGTT | 694 |
| NC_009657.1 | 22245 | CACTGCTAATGGCACCTTAGTTAATAACGGCACTGTGGTGTGTGTCACTT | 22294 |
| | | \|....\|.\|\|....\|.\|..\|..\|..\|. \|...\|.\|.\|.\|..\|..\| \|..\|. | |
| m64019_210618 | 695 | CGTACCAAAACAGAACGGAACTGTTT-CAATAATTTAGAATCAG-CGGTC | 742 |
| NC_009657.1 | 22295 | CTAATCAG---TTCACCCTTAGACACGACTTTATGGTAGGTTATTCTGCT | 22341 |
| | | \|\|\|..\|\|\| \|\|.\|...\|\|.\|\|... \|\|..\|.\|\|..\|........\|\|\|. | |
| m64019_210618 | 743 | CTATGCAGGAATTGAGTATTTGATTA-ACAATCTGTGAAAAATAAATGCA | 791 |
| NC_009657.1 | 22342 | GATATGCGTAAGGGTATATTTGAGTACTCTAGTACATGCCCTTTCAATAG | 22391 |
| | | .\|\|...\|....\|.\|\|..\|\|..\|..\|.....\|.\|.\|.\|\|...\|....\|.\|. | |
| m64019_210618 | 792 | AATGGACTGTGGTGTGAATAAGTTTTGAAAAATTCCTGAAGTGGGTAAAA | 841 |
| NC_009657.1 | 22392 | AGAAACTATCAATAACTACCTTACGTTTGGTCGTATTTGTTTCTCTACTT | 22441 |
| | | .\|\|..\|\|\| \|\|\|.\|\|.\|.\|\|..\| \|\|\|..\|...\|..\|\|...\|.\|.\|.\|\| | |
| m64019_210618 | 842 | TGAGTCTA--AATGACAAGCTGTC-TTTATTGCAAGCTGCGGCCCAATTT | 888 |
| NC_009657.1 | 22442 | CACCGGCGGACGGTGCTTGCGAATTGAAGTACTATGTTTGGAACACCATT | 22491 |
| | | .........\|\|\|..\|..\|.\|.\|\|...\|\|\|.\|.....\|\|\|..\|.....\|\|. | |
| m64019_210618 | 889 | TTGGATAAAACGTAGGATACCAAAGAAAGGAAAGATTTTACATACAAATA | 938 |
| NC_009657.1 | 22492 | GGAGCCGTTT-CACACCTGGCTGGCACCTTGTATGTTCAACATACAAAGG | 22540 |
| | | ....\|..\|.\| \|\|\|\|.\|....\|\|\|.\|.\|. \|.\|..\|..\|\|.\|\|.\|.\|\|\| | |
| m64019_210618 | 939 | TTCACATTATACACAACCATTTGGAAACA-GCAAATATAAAATCCCAAG- | 986 |
| NC_009657.1 | 22541 | GTGACATAATAACTGGTACACCCAAACCATTGCAGGGTTTGAATGACATT | 22590 |
| | | \|\|.\|.\|\|...\|\|\| \|.\|.......\|.\|.\|....\|\|\|\|.\|.\|..\|.\|. | |
| m64019_210618 | 987 | -TGCCCTATAGACT---AAATGTGTCTCCTGGATATGTTTAATTTGCCTC | 1032 |
| NC_009657.1 | 22591 | TCTGAATTGCACCTAGACACGTGCACCACTTACACCATTTATGGTTTTAG | 22640 |
| | | ..\|\|.\|\|\|\|..\|. \|\|.\|\|...\|\|....\|.....\|.....\|.\|..\|.. | |
| m64019_210618 | 1033 | CATGTATTGATCA---ACGCTGACAATTTTGGAGACTCACGTAGAATGTG | 1079 |
| NC_009657.1 | 22641 | GG-GTGACGGTGTTATTAGGTTGACCAATCAAACTTTCTTGTCAGGTGTC | 22689 |
| | | \|. \|\|.\|.\|.\|..\|.\|.\|.\|......\|......\|.\|\|\|\|\|\|.....\|..\| | |
| m64019_210618 | 1080 | GATGTCAAGCTTCTCTAAAGAATCAAACCTGGTCCTTCTTGATCACTTAC | 1129 |
| NC_009657.1 | 22690 | TA--CTACACTTCAGAGAGTGGTCAGTTATTAGCT--TTTAAGAATGTCA | 22735 |
| | | \|. \|\|...\|.\|...\|\|\|...\|.\|\|..\|.\|\|..\|\| \|\|\|...\|.\|\|\|\|. | |
| m64019_210618 | 1130 | TGTGCTGTGCCTTTTAGATCAGACATGTGTTCTCTAGTTTGCTACTGTCC | 1179 |
| NC_009657.1 | 22736 | CTACAGGGCAGATTTATTCTGTTACACCCTGCCAACTGGTTCAGCAGGTT | 22785 |
| | | \|.\|\| \|.\|..\|\|.\|\|\|..\|\|\|\|... .\|.\|\|.\|\|..\|\|...\|....\| | |
| m64019_210618 | 1180 | CCAC----CTGGGTTCTTCACTTACTTT-GGTCACCTTCTTTGTCTCCAT | 1224 |
| NC_009657.1 | 22786 | GCTTTTGTTGAGGATAGGATTGTTGGCGTC-ATTAGTAGTGCTAATAATA | 22834 |
| | | ...... \|.\|\|\|\|.\|\|.\|\|\|\|.\|\|\|.\|.\|. \|.\|\|...\|.\|.\|...\|..\| | |
| m64019_210618 | 1225 | AAGCAC-TGGAGGTTACGATTCTTGTCTTATAGTATACGAGGTCTGACAA | 1273 |
| NC_009657.1 | 22835 | CTGGGTTCTTTAATTCCA-CAAGAACATTTCCAGGCT-TCTATT------ | 22876 |
| | | .\|...\|\|\|.\|.\|\|\|\|\|.. \|.\|\|\|\|.\|..\|...\|..\| \|\|\|..\| | |
| m64019_210618 | 1274 | TTACATTCGTGAATTCATTCTAGAAAAAGTAATGCATATCTCATTGGTGA | 1323 |
| NC_009657.1 | 22877 | ATCACTCTAATGACACCACCAATTGCACCTCACCAAGACTTGTTTACTCT | 22926 |
| | | \|\|..\|.\|\|...\|.\|\|\|\|..\|\|\|. \|.\|\|..\|.\|...\|.....\|.\|.\|.\|\| | |
| m64019_210618 | 1324 | ATATCACTGTGGTCACCTTCAAA-GTACTCCCCTTGGGAAGCTGTGCACT | 1372 |
| NC_009657.1 | 22927 | AATATAGGTGTTTGTACTAGTGGTGCCATAGGTTTGCTGTCTCCTAAAGC | 22976 |
| | | .\|\|....\|.\|..\|...\|.\|....\|...\|....\|\|\|.....\|\|\|.\|..... | |
| m64019_210618 | 1373 | GATGCCAGCGCCTAGTCCACCCTTCAAAGCAATTTTGGAACTCTTTTCCT | 1422 |
| NC_009657.1 | 22977 | TGCACAA-CCTCAG-GTTCAACCCATGTT--CCAGGGTAATATTAGTATC | 23022 |
| | | .\|.\|... \|.\|\|\|\| \|.\|\|....\|.\|\|\|\| \|\|..\|.\|....\|.\|.\|.\|\| | |
| m64019_210618 | 1423 | GGAATGGTCATCAGAGCTCTCGTCGTGTTACCCTTGATGTCCTGAATGTC | 1472 |
| NC_009657.1 | 23023 | C-CTACTAATTTTACTATGAGTGTGCGCACTGAGTATATACAGTTGTTTA | 23071 |
| | | . \|.\|....\|\|\|\|.\|\|.\|.\|.\|.\|...\|..\| \|\|...\|\|\|.\|...\|. | |
| m64019_210618 | 1473 | ATCAAAATGTTTTCCTTTCAATATTTCCTTT----ATCATCAGGTAAAGA | 1518 |
| NC_009657.1 | 23072 | ACAAACCCGTTTCTGTAGACTGCGCAATGTATGTCTGCAATGGTAATGAC | 23121 |
| | | ...\|\|..\|.\|\| \|..\|.\|...\|..\|.\|\|..\|\|..\| .\|.\|\|\|..\| \| | |
| m64019_210618 | 1519 | GAGAAGGCATT---GGGGGCCAGGTGAAGTGAGTAGG-GAGGGTGTT--C | 1562 |
| NC_009657.1 | 23122 | CGTTGTAAGCAATTGTTGTCTCAGTACACTTCAGCATGCAAGAACATAGA | 23171 |
| | | \|..\|......\|.\|\|\|\|\|..\|\|...\|\|.\|......\|...\|\|........\|\| | |
| m64019_210618 | 1563 | CAATACGGTTATTTGTTTGCTGGTTAAAAACTCCCTCACAGACTGTGTGA | 1612 |
| NC_009657.1 | 23172 | ATCTGCGCTGCAGCTCAGCGCAAGGTTGGAATCAATGGAGGTTAACTCTA | 23221 |
| | | ..\|.\|.\|......\|.....\|\|\|\|..\|. .\|\|.\|\|\|.\|..\|...\|..\|.. | |
| m64019_210618 | 1613 | GCCGGTGTATTGTCATGATGCAAAATC--CATGAATTGTTGGAGAAACGT | 1660 |
| NC_009657.1 | 23222 | TGTTGACAGTTTCAGATGAGGCACTTAAGCTTGCCACTATAAGCCAATTT | 23271 |
| | | \|...\|.\|\|.\|\|\|\|...\|\|\|.\|...\|\|.\|....\|\|\|..\|......\|...\|. | |
| m64019_210618 | 1661 | TCAGGCCATTTTCGTCTGAAGTTTTTCACGCAGCCTTTTCTGCACTTCTA | 1710 |
| NC_009657.1 | 23272 | CCTGGTGG---TGGTTATAATTTTACCAATATTCTTCCAGCAAATCCTGG | 23318 |
| | | ..\|.\|\|.. \|\|\|\|\|\|....\|\|\|..\|.\|..\|..\|.\|.\|....\|\|..\|\|. | |
| m64019_210618 | 1711 | AATAGTAAACTTGGTTAACTGTTTGTCCAGTTGGTACAAATTCATAATGA | 1760 |
| NC_009657.1 | 23319 | -TGCTAGGTCAGTTATTGAAGACATTTTGTTCGATAAAGTTGTCACTAGT | 23367 |
| | | \|..\|...\|\|.\|.\|\|\|..\|\|.\|...\|..\|....\|\|....\|.\|.\|.\|\| \| | |
| m64019_210618 | 1761 | ATAATCCCTCTGATATCAAAAAAGGTCAGCAACATCGTTTGGACCCT--T | 1808 |
| NC_009657.1 | 23368 | GGTTTGGGCACAGTTGATGAAGATTATAAACGCTGCAGTAATGGACTGTC | 23417 |
| | | \|.\|\|\|\|\|.\| \|\|\|\|\|.\|..\|\|.\|.....\|......\|\|\|.\|.\|\|\|.\| | |
| m64019_210618 | 1809 | GATTTGGAC-----TGATGGAACTTTTTTCTTCGTGGAGAATTGGCTGAC | 1853 |
| NC_009657.1 | 23418 | TATTGCAGATTTAGCTTGTGCGCAGCACTATAACGGCATTATGGTGTTGC | 23467 |
| | | \| \|.\|\|..\|\|...\|\|\|...\|.......\|\|\|\|....\|\|\|..\|\|\|\|....\| | |
| m64019_210618 | 1854 | T--TCCATTTTGTACTTTGACATTCTGTTATAGGATCATATTGGTACACC | 1901 |
| NC_009657.1 | 23468 | CGGGTGTTGCGGACTGGGAAAAGGT--CCATATGTACTCGGCTTCACTTG | 23515 |
| | | \|..\|\|.\|......\|.\|.\|\|.\|\|..\| \|\|..\|..\|.....\|.\|.\|...\|. | |
| m64019_210618 | 1902 | CATGTTTCATCACCAGTGACAACATGGCCTAAAATGTCATGTTGCCTCTC | 1951 |
| NC_009657.1 | 23516 | TCGGTGGTATGACCTTAGGTGGTATCACTTCTGCTGCGGCTTTGCCTTTC | 23565 |
| | | .....\|\|\|.\|...\|..\|..\|\|\|..\|\|.\|\|\|.\|\|.\| \|\|\|\| \|\|\| | |
| m64019_210618 | 1952 | CAAAAGGTCTTGACAAACTTGGACTCTCTTTTGTT-----TTTG---TTC | 1993 |
| NC_009657.1 | 23566 | TCATATGCAGTGCAGGCAAGACTTAATTATGTTGCACTACAGACCGACGT | 23615 |
| | | .\|...\|\|...\|.\|.. \|..\|\|\|....\|\| \|\|\|\|\|.......\|..\|.\| | |
| m64019_210618 | 1994 | ACCGGTGAGCTACTT-CGGGACCATTTT----TGCACACATCTTCCTCAT | 2038 |
| NC_009657.1 | 23616 | GCTGCAACGTAATCAACAAATGCTAGCCAATTCCTTTAATAGTGCTATTA | 23665 |
| | | \|\| \|\|\|..\|..\|\|\|. \|.\|........\|\|.\|\|...\|\|.\|\|.\|.\|\|\| | |
| m64019_210618 | 2039 | GC--CAAGATTTTCAG----TTCAGATTTTGTCTTTCTCTATTGATTTTA | 2082 |
| NC_009657.1 | 23666 | GTAACATCACATTAGCTTTTGAGAGT--GTCAATAACGCTATCTATCAAA | 23713 |
| | | .......\|...\|..\|\|\|\|.\|.\|\|\|\|\| \|.\|\|\|\|\|\|\|..\|........\|. | |
| m64019_210618 | 2083 | CCTTGGACTACTATGCTTCTCAGAGTCAGCCAATAACTTTGATGGATCAT | 2132 |
| NC_009657.1 | 23714 | CTTCTGCTGGTTTGAATACGGTAGCAGAGGCACTTTCAAAAGTACAGGAT | 23763 |
| | | .\|..\|\|....\|\|\|\|.\|.....\|..\|\|...\|..\|\|...... \|\|..\|\|\|\| | |
| m64019_210618 | 2133 | TTGATGAATTTTTGCAATTTTTTTCATCAGTTCTACTCGT--TATTGGAT | 2180 |
| NC_009657.1 | 23764 | GTTGTGAATGGTCAAGGAAATGCACTCAGTCAACTAACAGTCCAATTGCA | 23813 |
| | | \|...\|\|\|....\|\|......\|....\|.\|..\|\|. \|\|..\|.....\|\|..... | |
| m64019_210618 | 2181 | GCCCTGACCTCTCTTTATCAGTTGCACGTTCT-CTCCCCTCGGAAAAAAC | 2229 |
| NC_009657.1 | 23814 | GAATAATTTTCAAGCTATTTCCAATTCTATTGGTGACATTTA--TAGTAG | 23861 |
| | | \|..\|\|.\|...\|.\|\|.....\|.\|.\|\|\|.\|\|\|\|..\|\|.\|\|\|\|.. \|..\|\|\| | |
| m64019_210618 | 2230 | GTTTACTCCACTAGTACACTGCCATTTTATTCTTGGCATTATCCTCATAG | 2279 |
| NC_009657.1 | 23862 | GTTAGATCAGATAACTGCTGATGCGCAAGTTGACAGACTTATCACAGGTC | 23911 |
| | | ..\|.\|..\|..\|.\|..\|.\| .\|\|.......\|..\|\|..\|\|\|. .\|\|\|\|\|. | |
| m64019_210618 | 2280 | ACTTGGACTAACACGTCC--CTGATTTCACTTCCACTCTTG--CCAGGTT | 2325 |
| NC_009657.1 | 23912 | GGCTTGCAGCTCTTAATGCCTTTGTTGCACAGTCACTTACCAAGTATGCA | 23961 |
| | | ..\|....\|..\| \|\|\|\|\|\| \|\|\|\|\|\| \|\|..\|\|.......\|\|\|\|.. \|\| | |
| m64019_210618 | 2326 | TACCAAGAAAT-TTAATG--TTTGTT-CATTGTTCTAATTCAAGCT--CA | 2369 |
| NC_009657.1 | 23962 | GAAGTGCAAGCTA-GTAGGACATTGGCCAAGCAAAAGGTTAACGAGTGT- | 24009 |
| | | \|\|..\|.\|..\|\|.\| \|.\|..\|\|\|....\|..\|..\|.\|\|...\|\|\|.\|\|.\|\|. | |
| m64019_210618 | 2370 | GACATTCTTGCGATGCAACACAAAAACACACAACAACAATAATGAATGCC | 2419 |
| NC_009657.1 | 24010 | GTTAAGTCACAGTCCCCCAGAT----ACGGTTTCTGTGGTGATGAAGGGG | 24055 |
| | | .\|\|.\|\|..\|\|\|...\|\|.\|\|..\| \|\|\|\|...\|.\|..\|\|\|\|........ | |
| m64019_210618 | 2420 | ATTCAGCAACACCGCCACATGTCCACACGGACACAGCTGTGAGATTTATA | 2469 |
| NC_009657.1 | 24056 | AACATA--TTTTCTCACTCACCCAAGCTGCTCCACAGGGTCTGATGTT-C | 24102 |
| | | .\|\|..\| \|\|.\|...\|\|.....\|.\|\|\|\|\|.\|......\|..\|\|\|....\| . | |
| m64019_210618 | 2470 | TACCAAGGTTATGAAACCTTATCGAGCTGTTTGTACAGTGCTGCCAATGT | 2519 |
| NC_009657.1 | 24103 | CTACACACCGTTTTAGTACCTAATGGTTTTATTAACGTTACAGCAGTTAC | 24152 |
| | | ..\|\|.\|\|..\|\|...\|....\|\|.....\|\|...\|\|.\|.\| \|\|..\|\|..\|.. | |
| m64019_210618 | 2520 | AAACGCAAGGTGGCAAGTTCTCGAACTTAATTTTAAG--ACCCCATATTT | 2567 |
| NC_009657.1 | 24153 | AGGTTTATGTGTTGATGAGACCATAGCTATGACATTACGTCAGAGTGGAT | 24202 |
| | | .\|....\|..\|.\|..\|......\|\|...\|. \|.\|\|.\|.. \|\|...\|\|.\|\|. | |
| m64019_210618 | 2568 | TGACAAACATTTCAAGATTTTCAATACA--GTCAATGT-TCTATGTTGAC | 2614 |
| NC_009657.1 | 24203 | TTGTCTTGTTTGTGCAAAATGG-TAATTATCTCGTG-TCACCGAGGAAAA | 24250 |
| | | \|\|.\|..\|\|..\|.\|...\|..\|.. \|..\|\|.\|.\|.\|\|\| \|......\|\|.\|\|. | |
| m64019_210618 | 2615 | TTATTATGAGTATTTTATTTAAATTTTTTTATTGTGCTATATAGGGGAAC | 2664 |
| NC_009657.1 | 24251 | TGTTTGAACCTCGGAGACCTGAAGTTGCTGATTTTGTGCAAGTAAAAACA | 24300 |
| | | .\|\|.\|\|...\|\|\|...\|.\|\|........\|..\|.\|...\|\|\|...\|.\|\|...\| | |
| m64019_210618 | 2665 | AGTGTGTTTCTCCAGGGCCCATCAGCTCCAAGTCATTGCCCTTCAATCTA | 2714 |
| NC_009657.1 | 24301 | TGCACGATTAGTTATGTTAACATCACCAATAACCAGTTGCCTGACATTAT | 24350 |
| | | .\|...\|....\|....\|.\| \|\|.\|.\|\|\|\| ..\|\|\|\|\|.\|\|\|.....\|.\|. | |
| m64019_210618 | 2715 | GGTGTGGAGGGCACAGCT--CAGCTCCAA-GTCCAGTCGCCGTTTTTCAA | 2761 |
| NC_009657.1 | 24351 | TCC--AGATTATGTAGACGTTAATAAGACTATAGATGAGATTTTGGCCAA | 24398 |
| | | \|\|. \|\|.\|...\|..\|.\|\|......\|...\|........\|...\|\|\|..\|.\| | |
| m64019_210618 | 2762 | TCTTTAGTTGCAGGGGGCGCAGCCCACCATCCCATGCGGGAATTGAACCA | 2811 |
| NC_009657.1 | 24399 | CCTACCTAATAATACTGTGC---CTGATTTGCCACTTGATGTCTTTAATC | 24445 |
| | | .\|.\|\|\|\|..\|..\|...\|.\|\| \|.\|..\|..\|\|\|..\|\|\| \|.\|.\|\|\|..\| | |
| m64019_210618 | 2812 | GCAACCTTGTTGTTGAGAGCTCACAGTCTAACCAACTGA-GCCATTAGGC | 2860 |
| NC_009657.1 | 24446 | AAACATTTCTTAATCTCACTGGTGAGATTGCAGACCTTGAAGCGCGATCT | 24495 |
| | | .\|.\|....\|. \|\|.\|..\|.\|\|.\|.\| \|\|.\|\|\|\|..\|...\|..\|..\|..\| | |
| m64019_210618 | 2861 | CACCCCAACA-AAACGTATTGTTTA--TTTCAGAAGTGATACAGAAAATT | 2907 |
| NC_009657.1 | 24496 | GAATCCCTTAAAAACACATCAGAAGAACTTAGACAGTTGATCCAAA-ATA | 24544 |
| | | ...\|......\|.\|\|.\|.\|\|\|\| ..\|.\|\|..\|.....\|\|\|\|\|\|\| \|.\| | |
| m64019_210618 | 2908 | AGGTGAAAAGAGAAAAAATCA----TTCATATTCCCAATATCCAAAGACA | 2953 |
| NC_009657.1 | 24545 | TTAACAACACACTTGTAGACCTTCAGTGGCTTAATAGGGTTGAGACCTTT | 24594 |
| | | ..\|\|\|\|...\|\|\|\|..\|..\|\|.\|\|........\|.........\|.\|.\|.\|.\| | |
| m64019_210618 | 2954 | AAAACACAGCACTGCTTCACATTTTAATAAATTTCCTTAAAGTGTCTTCT | 3003 |
| NC_009657.1 | 24595 | ATTAAGTGGCCGTGGTACGTGTGGTTGGCTATTGTTATAGCTCTTATTTT | 24644 |
| | | .\|\|.\|.\| \|..\|...\|....\|...\|\|.\|\|\|..\|.....\|..\|\|\|\|. | |
| m64019_210618 | 3004 | CTTTATT-----TAATCTCTACACTACACTTTTGAAAACTGACAAATTTA | 3048 |
| NC_009657.1 | 24645 | GGTTGTTTCACTGCTTGTGTTCTGCTGTATATCTACAGGTTGTTGCGGTT | 24694 |
| | | \|\|\|\|...\|\|.\|\|\|....\|\|.\|.\| \|\|...\|.\|\|\|....\|..\|\|\| \|\| | |
| m64019_210618 | 3049 | GGTTTAGTCTCTGGCAATGATTT-CTCCCTGTCTTTTAGAAGTT----TT | 3093 |
| NC_009657.1 | 24695 | GTTGCGGTTGTTGTGGTTCTTGTTTCTCAGGTTGTTGTCGTGGAACTAAA | 24744 |
| | | .\|\|\|...\|.....\|\|..\|.\|\|... \|...\|\|\|..\|..\|\|... ..\|\|.\|. | |
| m64019_210618 | 3094 | CTTGTACTGTGCCTGACTATTAAA-CATTGGTATTCTTCAAC-TTCTGAC | 3141 |
| NC_009657.1 | 24745 | CTT---CAACATTACGAACCAATAGAAAAGGTTCATGTGCAATAATGTTT | 24791 |
| | | \|\|\| \|..\|.\|...\|...\|\|.\|..\|.\|.\|.\|..\|\|.\|\|\|.....\|.\|.\| | |
| m64019_210618 | 3142 | CTTAAACCCCTTCTAGTCTCACTCAATATGATCAATATGCCCGGCTTTCT | 3191 |
| NC_009657.1 | 24792 | CTTGGTCTGTTCCAGTATACTATTGATACTGCAGTTGAGCACA-CTGTAG | 24840 |
| | | \| \|..\| \|..\|...\|\|..\|.\|..\|...\|.\|..\|...\|. \|\|.\|\|. | |
| m64019_210618 | 3192 | C-----CCAT--CTATGAGCTGATAAATCCCAAATAAACATCTTCTATAT | 3234 |
| NC_009657.1 | 24841 | AACATGCTAACTTGTCCCAAGAAGAGGCTTTGATGTTGGAAGAAAACATC | 24890 |
| | | \|...\|.\|\|..\|\|..\|\|...\| \|\|.....\|\|\|....\|\|.....\|.\|.\|\|\|. | |
| m64019_210618 | 3235 | ACACTACTTTCTGATCATTA-AATCCATTTTTCCATTTCCTTACAGCATA | 3283 |
| NC_009657.1 | 24891 | GTTCCTCTGAGACAAGCTACACATGTTACTGGATTTTTGCTCACCAGTGT | 24940 |
| | | .\| \|\|.\| \|.\|.....\|\|..\|.\|\|.\|.\|.. \|\|...\|\|\|...\|\|..\|.. | |
| m64019_210618 | 3284 | AT-CCAC-GTGGATGTCTTTAAATTTAAAC--ATCCATGCCTGCCCTTTC | 3329 |
| NC_009657.1 | 24941 | TTTTGTTTACTTCTTTGCACTGTTTAAGGCTTCAAGCTACA-AACGTAAT | 24989 |
| | | \|\|.\|\|\|...\|\|..\|..\|....\|\|\|\| \|..\|.\|\|\|\|..\|.\| \|\|.\|\|.\|. | |
| m64019_210618 | 3330 | TTCTGTACTCTCTTCAGATTAGTTT--GATTGCAAGTAAGAGAAAGTCAA | 3377 |
| NC_009657.1 | 24990 | TTGCTGCTATTTTTAGCACGTTTGTTAGCTTTATTAATTTATGCACCCAT | 25039 |
| | | ...\|...\| \|.\|.\|\|\|.\|........\|.\|. \|..\|\|.\|.\|......\|.\| | |
| m64019_210618 | 3378 | AATCAAAT-TGTGTAGAAAAAACAAAAACA--AAAAACTCAAAATAACCT | 3424 |
| NC_009657.1 | 25040 | TTTAATATTTTGTGGTGCATACTTGGACGCTTTTA-TAGTAGTCGCAACA | 25088 |
| | | \|\|\|..\|.....\|....\|..\|..\|\|\|\|\|.\|\|..... \|...\|\|\|\|.\|\|... | |
| m64019_210618 | 3425 | TTTGGTTCCAGGATAAGACTCGTTGGATGCAGAAGCTCCAAGTCTCACTG | 3474 |
| NC_009657.1 | 25089 | TTGACTTCTCGTCTATTGTTTTTGACCTACTACTCATGGCGTTATAAAAC | 25138 |
| | | .\|.\|.....\|.\|.\|.\|..\|\|\|...\|..\|.\|\|.\|\|\|\|\|..\| \|..\|..... | |
| m64019_210618 | 3475 | ATCATGCGCCATTTCTGTTTTGCTAGTTCCTTCTCATTTC-TCTTTTTTT | 3523 |
| NC_009657.1 | 25139 | TTATAAATTTCTTATTTACAACTCTTCCACACTTATGTTTTTACATGG-T | 25187 |
| | | \|\|.\|.\|\|\|\|\|\|...\|\|.\|.\|\|..\|\|.. .\|\|.\|\|.\|\|.....\|.\|\|\|. \| | |
| m64019_210618 | 3524 | TTTTTAATTTCACCTTCAGAATGCTGG-TCATTTGTGACCACAAATGACT | 3572 |
| NC_009657.1 | 25188 | CATGCCAATTATTATAATGGCAGGC--CCTATGTAATGCTTGAAGGTGGA | 25235 |
| | | ....\|\|\|..\|.....\|\|..\|\|\|.\|\| \|\|....\|....\|..\|...\|...\| | |
| m64019_210618 | 3573 | ACCACCATCTCCCTAAACTGCATGCTTCCAGATTCTAACCAGGCAGAAAA | 3622 |
| NC_009657.1 | 25236 | AGCCATTACGTCA-CATTGGGTACTGATATAGTACCATTCGTCAGCCGAA | 25284 |
| | | ...\|..\|.\|..\|. \|..\|...\|\|\|\|..\|.\|..\|\|..\|\|\| \|\|..\|\|.\|\| | |
| m64019_210618 | 3623 | GAACTGTGCAGCTTCTGTTTTTACTATTTTCCTAGTATT--TCCACCAAA | 3670 |
| NC_009657.1 | 25285 | GTAATCTCTATCTTGCCATTCGTGGTAGTGCTGAG-TCAGATATCCAACT | 25333 |
| | | \|\|..\|..\|..\|\|...\|...\| \|.\|.\|..\|\|\|.\|\| \|\|\|.\|\|.\|\|\|\|\|\|. | |
| m64019_210618 | 3671 | GTTTTGACAGTCACTCAGAT--TAGAACGGCTAAGGTCACATGTCCAACA | 3718 |
| NC_009657.1 | 25334 | GTTGAGAACTGTCGAGT---TGTTAGATGGTAATTAC--CTCTA-----C | 25373 |
| | | .\|..\|..\|....\|\|... .\|..\|\|\|\|\|..\|...\|\| \|\|\|\|. \| | |
| m64019_210618 | 3719 | CTGAACCAAATACGTTAGCCAGAGAGATGCAATGAACTGCTCTGTTTAGC | 3768 |
| NC_009657.1 | 25374 | ATTTTCTCCAGTTGTCAAGTCGTTGGTGTTACTAATTCAGGTTTTGAG-G | 25422 |
| | | ..\|\|\|\|.\|....\|..\|\|.\|.\|....\|.\|\|..\|\|....\|.\|\|.\|..\|\|\| \| | |
| m64019_210618 | 3769 | CGTTTCACATCATCGCAGGGCTCATGGGTACCTCCCACGGGCTGAGAGTG | 3818 |
| NC_009657.1 | 25423 | AGATTCAACTAGACGAATATGCTACAATTAGTGAATGATAATGGTGTAGT | 25472 |
| | | .\|....\|....\|\|\|..\|....\|.\|.....\|...\|..\|.\|..\|\|....\|.\| | |
| m64019_210618 | 3819 | GGGGAAAGAGGGACAGAACCTCAAATGAAACACAGAGCTGCTGTCAGAAT | 3868 |
| NC_009657.1 | 25473 | TGTAAATGCGATTCTCTGGCTTTTTGTACTCTTTTTTGTGC-TAGTTATT | 25521 |
| | | .....\|...\|..\|.\|\|..\|..\|\|....\|.\|..\|...\|\|..\| \|..\|\|..\| | |
| m64019_210618 | 3869 | AAAGCAAATGGATGTCAAGAATTAACAAATAATACCTGACCCTCCTTTAT | 3918 |
| NC_009657.1 | 25522 | AGCATTACTTTCGTCCAAC---TTATAAACCTTTGTTTTACTTGCCACCG | 25568 |
| | | .\|.\|\|..\|..\|\|...\|\|.\| \|\|..\|\|\|.\|\|\|.\|..\|.\|..\|\|..\|\|\|. | |
| m64019_210618 | 3919 | TGAATGGCACTCACTCATCCAGTTCCAAAACTTGGCATCATGTGAGACCA | 3968 |
| NC_009657.1 | 25569 | GTTGTGTAATAACGTTGTTTATAAGCCTGTTGGAAAAGTATACGGAGTAT | 25618 |
| | | ..\|...\|...\|.\|..\|\|.\|\| \|\|......\|.\|..\|.\|..\|.\|..\|\| | |
| m64019_210618 | 3969 | CATTACTCTGACCTCTGCTT-----CCATAATCACATCTCTTTGTATGAT | 4013 |
| NC_009657.1 | 25619 | ACAAGTCTTATATGCGAATTCAACCCTTGACATCTGACATTATTCAAGTA | 25668 |
| | | .\|...\|.\|......\|.....\|...\|...\|\|\|...\|\|\|.\|\|\|\|.. \|.\|.\| | |
| m64019_210618 | 4014 | TCTCTTGTCTACCTCTTTCACTTACAAGGACCCTTGAGATTACA-ATGGA | 4062 |
| NC_009657.1 | 25669 | TAAACGAAAATGTCTTCGAACCAATCCGTTCCTGTAGAGGAGGTGATTAA | 25718 |
| | | \|..\|\|..\|.\|\|.. \|.\|.\|\|.\|\|\|\|\|....\|.\|.\|..\|.....\|.\|.\|.. | |
| m64019_210618 | 4063 | TCCACACAGATAA-TACAAAACAATCTCCCCATCTCAATAGTCTTAATTT | 4111 |
| NC_009657.1 | 25719 | ACACCTCAGAAATTGGAACTTTTCATGGAATATCATACTTACAATACTCT | 25768 |
| | | \|..\|.\|..\|.\|...\|\|..\|.\|\|\|....\|.\|\|\|...\|\|........\|\|... | |
| m64019_210618 | 4112 | AATCATTTGTACAAGGTCCATTTTGCTGTATAAAGTAACATGTTAACATA | 4161 |
| NC_009657.1 | 25769 | TAGTAGTGTTGCAGTATGGACATTACAAATATTCCAGGGTTCTCTATGGC | 25818 |
| | | \|...\|\|.\|.\|...\|..\|.\|....\|....\|....\|\|\|...\|\|.\|...\|\|.\| | |
| m64019_210618 | 4162 | TTTCAGGGATTAGGATTAGCACATTTTGAGGGGCCATTATTTTGCTTGCC | 4211 |
| NC_009657.1 | 25819 | TTAAAGATGGCCATTCTTTGGCTTCTTTGGCCACTTGTTCTGGCCCTTTC | 25868 |
| | | ..\|...\|.. ...\|\|\|\|\|\|.\|..\|\|.\|......\|.\|...\|\|... \|\|\|. | |
| m64019_210618 | 4212 | ACACCCACA-TATTTCTTTAGAATCATCTTTAGCATAACCTAAT--TTTA | 4258 |
| NC_009657.1 | 25869 | CATCTTTGATGCCTGGGCCAGTTTTAATGTTAATTGGGTTTTCTTCGCAT | 25918 |
| | | .\|..\|.\|\|.\| \|\|\|\|.....\|.\|\|.\|\|..\|...\|.\|.\|\|.\|\|\|\|..\|.\| | |
| m64019_210618 | 4259 | GAAATGTGTT--CTGGCATTATGTTTATTCTGGGTTGCTTCTCTTTACTT | 4306 |
| NC_009657.1 | 25919 | TCAGCATCCTAA-TGGCCTGCGTCACAGCTGT-GCTGTGGATTATGTACT | 25966 |
| | | .\|...\|..\|\|.. \|..\|\|\|.\|.....\|\|\|..\| .......\|.\|.\|\|\|.\|. | |
| m64019_210618 | 4307 | GCTTAACACTCTGTATCCTTCACTCTAGCACTCAACACCCACTCTGTCCC | 4356 |
| NC_009657.1 | 25967 | TTGT-TAACAGTATCAGGTTGTGGCGACGCACCCATTCTTGGTGGTCCTA | 26015 |
| | | \|..\| .\|\|\|..\|.\|.\|\|.\|\|.\|...\| \|..\|.\|.\|.\|.\|...\|..\|.\|.. | |
| m64019_210618 | 4357 | TCATGCAACTTTGTGAGTTTCTCATG-CAAAACAACTTTGATTTATTCAT | 4405 |
| NC_009657.1 | 26016 | CAATCCTGAAACGGACTCTATTCTGTCTGTCTCTGTGCTGGGTCGGCATG | 26065 |
| | | ...\|....\|\|.....\|.\|.\|.\|\|\|\|\|\|\|\|.\|.\|......\|\|......\|\|. | |
| m64019_210618 | 4406 | TTCTGAGCAATAATGCCCAACTCTGTCTGGCACAACCAAGGAAATTAATA | 4455 |
| NC_009657.1 | 26066 | TCTGCCTACCAATACTTGGTGCACCCACGGGCGTAACGCTCACACTGCTT | 26115 |
| | | ..\|......\|.\|.\|.\|......\|\|\|..\|...\|..\|\|.\|\|\|........\|\| | |
| m64019_210618 | 4456 | ATTATAGTTCTAGAGTCCTCTAACCATCAACCTAAAAGCTTGATAGTTTT | 4505 |
| NC_009657.1 | 26116 | AATGGCACATTGCTTGTAGAAGGCTATCAG-GTTGCT-ACTGGCGTACAG | 26163 |
| | | .....\|.\|\|...\|....\|..\|..\|\|...\|\| \|\|\|\|\|\| \|.\|\|....\|\|\|. | |
| m64019_210618 | 4506 | TGATCCCCAAATCCCAAATTAATCTCAAAGTGTTGCTGAGTGAATCACAA | 4555 |
| NC_009657.1 | 26164 | GTAAATAATTTACCTGGTTACGTAACAGTCGCCAAAGCTTCAACAACAAT | 26213 |
| | | ..\|\|\|\|.\|\|\|\|..\|...\|.\|....\|...\|.\|\|\|\|\|\|\|.\| \|\|.\| | |
| m64019_210618 | 4556 | TGAAATTATTTTACATTTGAAAGGAATTTGGCCAAAGTT-------CACT | 4598 |
| NC_009657.1 | 26214 | TGTCTACCAGCGTGTGGGACGTTCCATGAATGCAAATTCAAGTACTGGCT | 26263 |
| | | \|..\|...\|....\|.\|...\|....\|\|\|. ..\|\|...\|.\|.\|\|.\|...\|..\| | |
| m64019_210618 | 4599 | TTACCTTCTAAATTTCAAATAAGCCAA-TTTGACCACTGAATTTTAGTAT | 4647 |
| NC_009657.1 | 26264 | GGGCTTTCTTCGTGAAGTCCAAGCATGGCGACTACTATGCTGCTGCGAAT | 26313 |
| | | ....\|.\|..\|..\|\|. \|.\|...\|......\|.\|.\|\|\|\|.\|...\|... \|.. | |
| m64019_210618 | 4648 | TTAATATAATGATGT-GCCATTGTTCTTAGTCAACTAAGAAACAAA-ACA | 4695 |
| NC_009657.1 | 26314 | CCAACAGAGGTTGTAACAGATAGTGAGAAAATTCTACATTTAGTCTAAAC | 26363 |
| | | \|.\|\|.\|.\|..\|\|.\|.\|.\|.\|.\|\|\|...\|\|\|\|....\|.\|.....\|..\|\|\|. | |
| m64019_210618 | 4696 | CTAAAATACCTTTTTAAAAAGAGTTTAAAAAAAAAAAAAGAGCTTAAAAT | 4745 |
| NC_009657.1 | 26364 | AGAAACTTA-TGGCTTCTGTAAAATTCCAACCTCGTGGTCGTTCCAAGGG | 26412 |
| | | .....\|\|\|. \|..\|.\|\|\|\|\|.\|.\|.\|..\|...\|\|..\|..\| \|\|\|\|...\|. | |
| m64019_210618 | 4746 | GACTTCTTGGTTTCATCTGTTACAATGAAGTTTCAAGTGC-TTCCTGAGA | 4794 |
| NC_009657.1 | 26413 | ACGTGTTCCTCTGTCTCTTTTTGCTCCACTTAGGGTTACTGATGAAAAAC | 26462 |
| | | \|.\|...\|.\|\|..\|..............\|..\|..\|\|..\|.\|...\|\|..\|\|\| | |
| m64019_210618 | 4795 | AAGAAGTTCTAGGAAGAACAACTAAAAAACTGTGGACATTACAGAGCAAC | 4844 |
| NC_009657.1 | 26463 | -CACTTTACAAGGTCCTACCAAATAATGCCGTCCCTCAGGGAATGGGAGG | 26511 |
| | | \|....\|\|\|\|.\|.....\|\|.\|.\|...\|\|\|\|.\|..\|\|.....\|.\|..\|.\|. | |
| m64019_210618 | 4845 | TCTGAATACATGAATTGACAACAGTGTGCCTTAACTTTAATACTCTGTGT | 4894 |
| NC_009657.1 | 26512 | TAAG--GACCAACAAATTGGATACTGGGTTGAACAACAGCGCTGGAGAAT | 26559 |
| | | .\|.. \|\|\|\|.\|.\|..\|....\|.\|\|..\|.....\|..\| \|.\|\|\|. .\|..\| | |
| m64019_210618 | 4895 | CACATTGACCCAAATGTACCCTCCTCAGCCAGTCTTC-GAGCTC-TGTTT | 4942 |
| NC_009657.1 | 26560 | GCGCCGCGGAGACAGAGTTGACCTGCCATCTAACTGGCACTTCTACTTCC | 26609 |
| | | .\|.\|.. \|.\|.\|\|\|\|\|\|..\|....\|.\|........\|\|\|\|...\|.\|.\|\|. | |
| m64019_210618 | 4943 | TCTCAT--GGGTCAGAGTCAATTCTCAAATCGTAAAGCACACATCCATCT | 4990 |
| NC_009657.1 | 26610 | TCGGTACTGGACCGCATTCTGATTTGCCTTTCAGAAAACGCACTGATGGT | 26659 |
| | | .\|.\|.. \|\|\|.\|..\|\|\| \|\|.\|.\|....\|.\|....\|\|\|...\| | |
| m64019_210618 | 4991 | GCAGAT-------GCAATGGGAT---CCCTACCAGCATCAATGTGAGCCT | 5030 |
| NC_009657.1 | 26660 | GTTTTCTGGGTTGCA-ATCGATGGTGCTAAGACCCAGCCAACAGGCCTTG | 26708 |
| | | ..\|..\|\|\|.....\|\| \|\|\|..\|.\|\|.\|.\|..\|\|\|. \|\|......\|\|... | |
| m64019_210618 | 5031 | TATGGCTGAAAGACATATCAGTAGTCCAATCACCA--CCCTTGTACCCGC | 5078 |
| NC_009657.1 | 26709 | GCGTACGTAAGTCGTCTGAGAAGCCGTTGGTTCCAAAATTTAAGAACAAA | 26758 |
| | | .\|.\|.\|.\|..\|..\| \|\|..\|\|\|\|\|.\|\|......\|\|.\|\|.\|....\|.\|... | |
| m64019_210618 | 5079 | CCTTTCATTGGAAG-CTCTGAAGCAGTCTCCCTCATAAGTGTGAACCTTG | 5127 |
| NC_009657.1 | 26759 | TTACCCAATAATGTGGAAATCGTTGAACCTACCACACCAAACAACTCCAG | 26808 |
| | | ..\|...\|\|\|\|\|\|.\|\|..\|\|........\|\|\|........\|...\|.\|\|\|.\|. | |
| m64019_210618 | 5128 | AGAAGAAATAATCTGCCAAGAAGGATTCCTCATGGTTAACTGAGCTCAAA | 5177 |
| NC_009657.1 | 26809 | AGCTAACTCAAGGAGTCGTAGTCGTGGTGGACAGTCCAACAGCAGAGGAA | 26858 |
| | | ..\|\|.\|.\|..\|\|.. \|...\|\|.\|.\|....\|\|\|\| \|.\|.\|..\|......\| | |
| m64019_210618 | 5178 | TTCTTAATAGAGTC-TAACAGCCATTCCTGACA--CAAGCCTCTCGCACA | 5224 |
| NC_009657.1 | 26859 | ATTCCCAAAACAGAGGT--GATAAATCCAGAAA---CCAGTCCAGAAACA | 26903 |
| | | .\|...\|\|...\|\|\|.\|.. \|..\|.\|..\|.\|\|\|\| \|\|\|...\|...\|\|\|. | |
| m64019_210618 | 5225 | CTCTGCATTTCAGGGAAAAGCCACAGACTGAAATTTCCACCTCCCGAACT | 5274 |
| NC_009657.1 | 26904 | GGAGTCAATCTAATGATCGTGGGTCTGACTCGCGAGATGACTTAGTGGCT | 26953 |
| | | \|...\|\|...\|\|..\|\| \|.\|..\|\|\|...\|..........\|\|\|\|..\|\|... | |
| m64019_210618 | 5275 | GTGCTCCTGCTGCTG--CCTAAGTCAACCATTGTCAGGAACTTCCTGATG | 5322 |
| NC_009657.1 | 26954 | GCCGTTAAAAAAGCACTT--GAAGACCTAGGAGTTGGTGCTGCAAAGCCA | 27001 |
| | | \|...\|....\|..\|.\|\|\|\| .\|\|\|..\|\|..\|\|.\|.\|...\|..\|.\|\|.\|.. | |
| m64019_210618 | 5323 | GAACTCCTGATGGAACTTCCAAAGGACTGAGACTAGTCCCATCCAATCAG | 5372 |
| NC_009657.1 | 27002 | AAA---GGC---AAAACCCAGAGTG-GTAAAAAC--ACCCCTAAGAACAA | 27042 |
| | | \|\|. \|\|\| \|.\|.\|\|.....\|\| .\|..\|.\|\| \|\|\|..\|.\|\|\|\|\|.. | |
| m64019_210618 | 5373 | AACTGTGGCGTTATATCCTCATTTGCATCTATACTGACCAATCAGAACTG | 5422 |
| NC_009657.1 | 27043 | ATCTAGGTCAGGCTCTGTGCA-ACGTGCAGAAGCCAAGGACAAACCCGAG | 27091 |
| | | \|\|..\|...\|\|..\|..\|..\|.\| \|..\|\|..\|..\|.\|. \|..\|\|.\|.\|.\|.\| | |
| m64019_210618 | 5423 | ATTCACAACAACCAATCAGAACATATGATGCTGACT-GATCAGAACTGTG | 5471 |
| NC_009657.1 | 27092 | TGGCGTCGTACTCCTAGTGGCGATGAGTCAGTTGAGGTTTGTTTTGGACC | 27141 |
| | | \|\|...\|.\|...\|\|..\|.\|.\|\|....\|....\|...\|..\|..\|.....\|..\| | |
| m64019_210618 | 5472 | TGATTTGGATTTCTCATTTGCATAAAAATGGACCAAATGGGAACCAGGGC | 5521 |
| NC_009657.1 | 27142 | CCGTGGTGGCACCAGAAATTTTGGTAGCTCCGAATTTGTTGC-TAAAGGT | 27190 |
| | | .\|....\|..\|.\|...\|\|\|.........\|.\|\|....\|..\|.\|. \|..\|..\| | |
| m64019_210618 | 5522 | ACTAACTTTCTCTGTAAAAGGCCCCTTCCCCTTTGTCTTGGTGTGCACTT | 5571 |
| NC_009657.1 | 27191 | GTGAATGCCCCCGGTTATGCTCAG----GCTGCTTCACTGGTACCCGGCG | 27236 |
| | | ..\|..\|...\|\|.\|.\|\|\|....\|.\| \|\|\|\|..\|.\|....\|\|.\|\|.... | |
| m64019_210618 | 5572 | TCGGTTTTTCCTGTTTACCAACTGTTCAGCTGAATAAAGTTTATCCTCTT | 5621 |
| NC_009657.1 | 27237 | CCGCAGCACTGCTTTTTGGTGGTAATGTTGCCACCA---AGGAAATGG-- | 27281 |
| | | .\| \|\|...\|\|..\|.\|\|.\|....\|..\|\|\|\|\|..\|..\| \|\|\|..\|\|\|\| | |
| m64019_210618 | 5622 | TC-CACACCTCATATTGGAAACTTTTGTTGATATGAGGTAGGCTATGGTC | 5670 |
| NC_009657.1 | 27282 | CTGATGGTGTTGAAATCACCTATACATATAAAATGTTAGTCCCTAAGGAC | 27331 |
| | | ...\|\|......\|\|...\|.\|.\|..\|..\|.....\|..\|.\|.\|..\|.\|\|..\|\| | |
| m64019_210618 | 5671 | ACAATTCACAAGAGGACCCTTGAAGCTCAGTGAGATGATTTTCAAATAAC | 5720 |
| NC_009657.1 | 27332 | GACAAGAACCTTGAAATCTTTCTTGCTCAGGTTGACGCATACAAGCTCGG | 27381 |
| | | ...\|.\|\|..\|.\|..\|.....\|.\|\|\|...\|\|\|..\|\|.\|........\|..\|\| | |
| m64019_210618 | 5721 | AGGAGGATTCATCCAGATGATTTTGAGAAGGAAGAGGTTACTGCCCCAGG | 5770 |
| NC_009657.1 | 27382 | CGATCCCAAGCCTCAGCGTAAAGTCAAACGTTCAAGAACCCCAACACCAA | 27431 |
| | | .\|.\|\|.\|.\|. .\|.\|\|.\|.\|\|\|...\|....\|.\|......\|.\|..... | |
| m64019_210618 | 5771 | AGTTCACTAA----GGAGTGAGGTCTGCCAAAAACGTGAAAAAGCTGTGT | 5816 |
| NC_009657.1 | 27432 | AACCTGCAACAGAGCCAGTTTA-TGACGACGTTGCTGCAGATCCTACTTA | 27480 |
| | | \|....\|\|....\|.\|..\|.\|\|.\| \|\|...\|\|..\|. ...\|.\|....\|\| | |
| m64019_210618 | 5817 | AGATGGCCCTCGTGATATTTCAGTGGAAACAATA----TATTTCATTGTA | 5862 |
| NC_009657.1 | 27481 | CGCCAATCTTGAGTGGGACACCACAGTGGAGGATGGTGTTGAGATGATCA | 27530 |
| | | .\|...\|....\|.\|.\|\|\|...\|.\|....\|\|\|\|\|\|\|\|.\|\|.\|\|....\|\|\|.. | |
| m64019_210618 | 5863 | GGTTGAAAGAGTGAGGGTATCTAACAGGGAGGATGCTGCTGGACAGATTT | 5912 |
| NC_009657.1 | 27531 | ACGAGGTTTTTGACACCCAGAATTGAATTCAACTAAAACAATGTACAGAA | 27580 |
| | | .....\|\| \|.\|\|\|.\|.......\|\|..\|\|.\| \|.\|\|...\|\|.\|\|.\|\| | |
| m64019_210618 | 5913 | CTCCAGT----GTCACACTACCGCCAAGACAGC--ACACGGAGTTCACAA | 5956 |
| NC_009657.1 | 27581 | TTGTAGCTATTGTTTTGGCTGAGCTTTTTCGAGCACTGGCCATTTTTGGC | 27630 |
| | | ..\| \|...\|.\|\|.\|.\|\|\|.\|\|.\|..\|......\|\|\|.\|...\|.\|\|\|\|\| | |
| m64019_210618 | 5957 | AAG-ACAAAATGGTATGGTTGTGAGTAGGAATGCATTTTTCTTTTTT--- | 6002 |
| NC_009657.1 | 27631 | TCATTCTTCCAAATTTTTTTGCTATATTTTGATTGCATTTCCAAGGTGAG | 27680 |
| | | ..\|\|\|\|\|\|\|...\|\|\|\|\|\|\|..\|.\|.\|\|\|.\|..\|\|\|\|\|\|\|....\|.\|.\|. | |
| m64019_210618 | 6003 | -TTTTCTTCCTTTTTTTTTTTTTTTTTTTGGTATGCATTTTTCTGATTAA | 6051 |
| NC_009657.1 | 27681 | TTTAAGCTGTCCTACAGGACGTTGGTGTTTGCTTACATGTGCTGATTTCC | 27730 |
| | | \|.\|.....\|...\|.\|.....\|\|....\|..\|\|...\|\|\|..\|\| \|\|.\|\|\|. | |
| m64019_210618 | 6052 | TCTTCTTGGAGTTGCTCATTGTCACAGCATGAAAACACCTG--GAATTCT | 6099 |
| NC_009657.1 | 27731 | TTATTCTTGTGC-TCATATTCTTTCTTTTCTTGGTGCCTTTTTCTTACTG | 27779 |
| | | \|..\|\|\|.\|\|... \|..\|..\|...\|\|\|\|\|..\|..\|. ..\|\|\|.\|.\|\|.\|\|. | |
| m64019_210618 | 6100 | TGTTTCCTGACAGTGCTTATAGATCTTTGATCAGC-TATTTATGTTGCTC | 6148 |
| NC_009657.1 | 27780 | TTTAGTGGTGTACATCGTTAA-AGATGATTGGGCCCCCTGGATGTGGTAT | 27828 |
| | | ..\|.......\|.\|\|\|.\|..\|\| \|\|..\|\|\|...\|\|...\|\|.\|.\|.\|..\|.\| | |
| m64019_210618 | 6149 | AATGTCCACTTCCATAGAAAACAGTAGATGCAGCAGTCTAGTTCTCATTT | 6198 |
| NC_009657.1 | 27829 | GTTAACCTCTACAGGCCCCTACATGATGCCTTAATCAGATTTCTTATG-A | 27877 |
| | | \|.\|...\|\|\|..\|\|.........\|...\|\|..\|..\|\|\|.\|\|\|....\|.\|. \| | |
| m64019_210618 | 6199 | GCTCCACTCATCAAATTAACCAAGTCTGTATCTATCTGATGATGTGTATA | 6248 |
| NC_009657.1 | 27878 | CACCAGACTTTGCTGTCTTGGTTTTATCTTTCTTGTTCATGATCTTAACA | 27927 |
| | | .\|...\|..\|.\|\|.\|\|..\|\|.\|.\|\|.\|.\|\|\|..\|........\|.\|...\|\|. | |
| m64019_210618 | 6249 | TATGTGTGTGTGGTGCATTAGATTCAGCTTGTTGACAATAAAACAATACT | 6298 |
| NC_009657.1 | 27928 | TG-GCTGCTGGGCATTGGAATCTTCCAATACTAGCGGT-CTTGGTCTTGC | 27975 |
| | | \|. ..\|\|...\|\|.\|\|....\|.\|\|.\|...\|\|...\|.\|.\| \|.\|..\|....\| | |
| m64019_210618 | 6299 | TTTATTGACTGGGATACTGACCTACTTGTATATGTGCTGCCTCTTTAAAC | 6348 |
| NC_009657.1 | 27976 | ACACAACGGTAAGCCTGTAATAATGACAGTGCAAGCAGGTTATTATTATA | 28025 |
| | | ...\|.\|\|..\|....\|..\|\|..\|..\|.......\|\|..\|.....\|....... | |
| m64019_210618 | 6349 | CTTCCACTTTGTTTCAATAGAATAGTATAAAAAACAAAAAGCTCTAGGAT | 6398 |
| NC_009657.1 | 28026 | TTGC | 28029 |
| | | \|\|\|\| | |
| m64019_210618 | 6399 | TTGC | 6402 |

TABLE 4
Alignment of identified sequence with the RaTG13 bat coronavirus
genomic sequence

| Sequence 1 | MN996532.2:21560-25369 Bat coronavirus RaTG13, complete | | |
|---|---|---|---|
| | genome (SEQ ID NO: 354) | | |
| Sequence 2 | hub_1489433_GCA_004115265.2_dna (SEQ ID NO: 355) | | |
| Matrix | EBLOSUM62 | | |
| Gap penalty | 16 | | |
| Extend penalty | 4 | | |
| Length | 3998 | | |
| Identity | 1758/3998 (44.0%) | | |
| Similarity | 1758/3998 (44.0%) | | |
| Gaps | 281/3998 (7.0%) | | |
| Score | 6062 | | |
| 21560-25369 | 8 | TTTTTCTTGTTTTATTGCCACTAGTTTCTAGTCAGTGTGTTAATCTAACA | 57 |
| | | \|\|.\|\|\|....\|.\|\|\|\|.\|...\|.\|..\|.\|...\|\|.\|....\|.\|.....\|\| | |
| hub_1489433_G | 134 | TTGTTCACTATGTATTACATATTGAATTTTCACAATAATGTTAAGAGGCA | 183 |
| 21560-25369 | 58 | ACTAGAACTCAGTTACCTCCTGCATACACCA---ACTCATCCACCCGTGG | 104 |
| | | ..\|\|.\|\|.\|.\| \|\|\|\|\|\|\|. \|.\|.\|\|... \|....\|....\|.\|.\|. | |
| hub_1489433_G | 184 | GGTACAATTAA--TACCTCCA-CTTTCAGATGAGAAAATTAAGGCAGAGA | 230 |
| 21560-25369 | 105 | TGTCTATTACCCTGACAAAGTTTTCAGATCTTCAGTTTTACATTTAACTC | 154 |
| | | .\|\|....\|\|...\|\|.\|.\|\|\|.\|..\|\|.\|.\|\|\| \|.\|..\|\|\|.. \|.\|\|. | |
| hub_1489433_G | 231 | GGTTACATAATGTGCCCAAGGTACCACACCTT--GATAAACAGC-AGCTG | 277 |
| 21560-25369 | 155 | AGGATTTGTTTTTACCTTTCTTCTCCAA----TGTG-ACCTGGTTCC--- | 196 |
| | | .\|..\|.\|.......\|\|..\|\|..\|\|.\|\|. \|\|\|\| \|\|.\|...\|.\| | |
| hub_1489433_G | 278 | GGATTCTCACCCATCCAGTCAGCTTCAGAATCTGTGCACTTAACTACTAG | 327 |
| 21560-25369 | 197 | ATGCTATACATGTTTCAGGGACCAATGGTATTAAAAGGTTTGATAACCCA | 246 |
| | | \|\|\|\|\|\|\|\|.\|...\|\|.\|.\| \|\|\|\|...\|.\|.\|\|\|\|.....\|..\|....\| | |
| hub_1489433_G | 328 | ATGCTATATAGAATTAATG--CCAAAACTCTCAAAATCAGAGTCATGAGA | 375 |
| 21560-25369 | 247 | GTTCTGCCATTCAACGATGGCGTCTATTTTGCTTCCACTGAGAAGTCTAA | 296 |
| | | \|....\|\|\|\|. \|.\|.\|\|...\|.\|.\|\|.\|\|..\|\|....\|..\|.......\| | |
| hub_1489433_G | 376 | GAAAAGCCAA--AGCCATCATGCCAATATTTGTTAGGTTAGGTTAGGCTA | 423 |
| 21560-25369 | 297 | TATAATAAGAGGATGGATTTTTGGTACTACCTTAGATTCGAAGACCCAGT | 346 |
| | | \|.\|.\|.....\|..\|...\|\|\|\|\| \|.\|.\|\|.\|\|..\|\|\|..\|..\|. \| | |
| hub_1489433_G | 424 | TGTTAGGTTCGTTTTATTTTTT---ATTCCCCTAATTTCCTAATCT---T | 467 |
| 21560-25369 | 347 | CTCTACTTATTGTTAATAACGCTACTAATGTTGTTATTAAAGTCTGTGAA | 396 |
| | | \|\|..\|.\|\|\|..\|..\|...\|.\| \|.\|\|..\|.\|..\|.\|\|.\|\|.\|\|.\|.\|\|\|\| | |
| hub_1489433_G | 468 | CTACATTTAGGGGAAGAGATG-TGCTTCTATATTCATGAATGTTTATGAA | 516 |
| 21560-25369 | 397 | TTTCAATTTTGTAATGATCCATTTTTGGGTGTTTATTACCACAAAAACAA | 446 |
| | | \|. \|\|..\|.\|\|\|..\|..\|\|\|\|\|.\|.....\|....\|.\|..\|.\|.\|..... | |
| hub_1489433_G | 517 | TG--AACATCGTATGGGACCATTATAACTGGACCCTAAGGAGATATGTTC | 564 |
| 21560-25369 | 447 | CAAAAGTTGGATGGAAAGTGAGTTCAGAGTTTACTCTAGTGCGAATAATT | 496 |
| | | ...\|..\|........\|.....\|.\|\|\|\|..\|\| \|\|\|\|. \|\|.\|...\|.\|. | |
| hub_1489433_G | 565 | TTGACATAATTCATTATCAATGATCAGCATT--CTCTT-TGGGTTGATTG | 611 |
| 21560-25369 | 497 | GCACTTTTGAGTATGTCTCTCAGCCTTTTCTTATGGACCTTGAAGGAAAA | 546 |
| | | \|\|..\|.\|........\|\|\|\|...\|.\|.\|.\|...\|..\|..\|\|\| \|.\|.\|.\|\| | |
| hub_1489433_G | 612 | GCCATGTCTTTATCATCTCCACGTCCTATAGAACTGTTCTT-ATGAAGAA | 660 |
| 21560-25369 | 547 | CAGGGTAATTTCAAAAATCTTAGGGAATTCGTGTTTAAGAATATTGATGG | 596 |
| | | .\|..\|\|.\|...\|\|.\|.\|.........\|..\|..\|.....\|.....\|\|..\|. | |
| hub_1489433_G | 661 | TATAGTCAGGACACACACACACATACACACACGCGCGCGCGCGATGGGGA | 710 |
| 21560-25369 | 597 | TTATTT-CAAAATATATTCTAAACATACGCCTATTAATTTAGTGCGTGAT | 645 |
| | | .\|.\|\|. \|.\|..\|.....\|.\|...\|.\|.\|\|\|\|..\|.....\|\|..\|....\| | |
| hub_1489433_G | 711 | CTCTTAACTAGCTCACCCCCACCAAAAAGCCTCATCTAGAAGCACAGAGT | 760 |
| 21560-25369 | 646 | CTTCCCCCTGGTTTTTCAGCTTTAGAAC--CATTGG--TAGATCTGCCAA | 691 |
| | | ..\|\|.........\|.\|..\|.....\|..\| \|\|\|.\|\| \|.\|...\|.\|.\|\| | |
| hub_1489433_G | 761 | TATCATGAAATACTCTGGGGGAGGGGGCATCATGGGGGTGGCAATTCAAA | 810 |
| 21560-25369 | 692 | TAGGTAT---TAACATCACTAGGTTTCAAACTTTACTTGCTTTACATAGA | 738 |
| | | .\|\|..\|\| .\|\|.\|\|\|\|\|.\|\|.\|.\|..\|\|.\|..\|...\|..\|..\|...\| | |
| hub_1489433_G | 811 | GAGAAATGAGAAAAATCACAAGATGTTTAAATCAATGGGGATAGCGCTG- | 859 |
| 21560-25369 | 739 | AGCTATTTGACTCCTGGTGATTCTTCTTCAGGTTGGACAGCTG-----GT | 783 |
| | | \|...\|\|\|...\|\|\|\|\|..\|\|\|\|.\|\|.....\|\|.\|..\|\|..\|\|\| \|\| | |
| hub_1489433_G | 860 | -GAATTTTCCATCCTGAAGATTTTTTCCAGGGCTAAACCTCTGACTGAGT | 908 |
| 21560-25369 | 784 | GCTGCAGCTTATTATGTGG-----GTTATCTTCAACCAAGGACTTTTCTA | 828 |
| | | ..\|\|...\|\|\|.......\|\| \|\|..\|..\|.....\|....\|\|\|.\|.\|. | |
| hub_1489433_G | 909 | TTTGTTTCTTTAACAAAGGAGGTGGTGGTGGTGGTAGATTACCTTATTTT | 958 |
| 21560-25369 | 829 | CTAAAATATAATGAGAATGGAACCATTACAGATGC--TGTAGACTGTG-C | 875 |
| | | \|.\|\|\|\|..\|..\|..\|.\|...\|.\|\|\|..\|....\|.\| \|\|.\|.\|.\|\|\|. \| | |
| hub_1489433_G | 959 | CAAAAACGTTCTTTGTAAACATCCAAAATTATTTCCATGAAAATTGTTTC | 1008 |
| 21560-25369 | 876 | ACTTGAC-----CCTCTTTCAGAAACAAAGTGTACGTTAAAATCCTTCAC | 920 |
| | | .\|\|\|... \|\|\|\|..\|...\|..\|\|.. \|\|..\|.\|...\|.\|.\|\|\|... | |
| hub_1489433_G | 1009 | TCTTACATGTGACCTCAATTGTACTCAGC-TGACCCTGTGACTACTTGGA | 1057 |
| 21560-25369 | 921 | TGTTGAAAAAGGAATTTATCAAACCTCTAACTTTAG--AGTCCAACCAAC | 968 |
| | | \|\|\|\|.....\|.\|.....\|..\|\|\|...\|..\|\|...\| \|\|\|\|...\|.... | |
| hub_1489433_G | 1058 | -GTTGTGGTGGAACAAAGTGCAACAGTTTCCTCCTGGAAGTCTTTCATTT | 1106 |
| 21560-25369 | 969 | AGATTCTATTGTTAGATTCCCAAATATTACAAA-----CTTATGTCCT-- | 1011 |
| | | ..\|\|\|.\|\|\|.....\|......\|\|\|.\|.\|\|\|\|.. .\|\|\|..\|... | |
| hub_1489433_G | 1107 | TCATTGTATGAGGTGTGATAAAAAAAATACAGTGAATGTTTAAATAAAAA | 1156 |
| 21560-25369 | 1012 | -TTTGGTGAAGTTTTTAACGCC--ACCA--CATTCGCATCAGTTTATGCT | 1056 |
| | | \|\|\|..\|..\|\|\|.....\|\|.\|. \|\|\|\| .\|\|\|\|.\|.\|\|\|...\|\|..\|. | |
| hub_1489433_G | 1157 | ATTTATTACAGTAAAAGACACATTACCATTAATTCTCCTCAAAATACTCC | 1206 |
| 21560-25369 | 1057 | ---TGGAACAGAAAGAGAATTAGCAACTGTGTT-GCTGATTACTCTGTCC | 1102 |
| | | \|.\|....\|\|\|......\|...\|...\|\|\|.\|\| \|\|...\|\|.\|\|....\|. | |
| hub_1489433_G | 1207 | CCCTTGCTTTGAACACATGTATCCCTTTGTTTTTGCCACTTTCTGAAGCA | 1256 |
| 21560-25369 | 1103 | TATAT--AATTCCACTTCATTTTCTACCTTTAAATGTTATGGAGTGTCTC | 1150 |
| | | ..\|.\| \|\|.\|\|\|.\|\|\|...\|...\|..\|\|\|\|\|\|.\|\|\|..\|\|.\|\|\|\|.\|\|. | |
| hub_1489433_G | 1257 | GTTCTGGAAGTCCTCTTTTATGAGTGTCTTTAATTGTACTGTAGTGGCTG | 1306 |
| 21560-25369 | 1151 | CTACTAAATTAAATGATCTCTGCTTTACTAATGTTTATGCAGACTCATTT | 1200 |
| | | \|\|. \|.\|..\|.....\|\|\|..\|.\|...\|....\|...\|.\|...\|. \|\|\|\|\|\| | |
| hub_1489433_G | 1307 | CTT-TGATGTCCTGAATCAATTCAAAAAGTTTACCTTTTGTGG-TCATTT | 1354 |
| 21560-25369 | 1201 | GTGATTACAGGTGATGAAGTCAGACAAATTGCGCCAGGACAAACTGGAAA | 1250 |
| | | .\|..\|\|...\|\|....\|....\|\|\|.\|..\|..\|.\|\|\|\|\|..\|....\|...\|\| | |
| hub_1489433_G | 1355 | TTTCTTTAGGGAAGAGCCAGCAGTCGCACGGTGCCAGATCTGGTTAATAA | 1404 |
| 21560-25369 | 1251 | GATTGCTGACTACAATTATAAACTACC--AGATGATTTTACTGGTTGTGT | 1298 |
| | | \|...\|.\|\|\|..\|\|\|......\|\|...\|. \|\|.\|\|\|....\|.\|.\|.\|\|\|.\| | |
| hub_1489433_G | 1405 | GGCGGATGAGGACACACCATAATGTCTTTAGTTGACAGAAATTGCTGTAT | 1454 |
| 21560-25369 | 1299 | TATAGCTTGGAATTCTAAGCATATTGATGCAAAAGAGGGCGGTAATTTTA | 1348 |
| | | ...\|\|....\|\|..\|...\|\|\|\|\|..\|.\|\|\| \|.\|\|\|\|\|..\|.\|....... | |
| hub_1489433_G | 1455 | ACCAGAAGCGATGTTGGAGCATTGTCATG--ATAGAGGATGATTTACAGC | 1502 |
| 21560-25369 | 1349 | ACTATCTTTACCGTCTCTTTAGAAAAGCTAATCTTAAACCCTT-TGAGAG | 1397 |
| | | \|\|..\|.\|..\|.\|....\|\|\|.....\|\|\|...\|\|\|\|.\|.\|\|\|\|.. \|\|\|..\| | |
| hub_1489433_G | 1503 | ACACTGTAAAACACACCTTCTCTCAAGTGTATCTCACACCCAACTGACTG | 1552 |
| 21560-25369 | 1398 | GGATATCTCAACTGAAATTTACCAAGCA--GGCAGCAAACCTTGTAATGG | 1445 |
| | | ....\|....\|..\|\|\|\|\|.\|\|..\|\|..\|\| ...\|..\|\|...\|\|\|..\|\|\|. | |
| hub_1489433_G | 1553 | CACCAAACAAGTTGAAACTTGTCACACATCATTACTAAGGTTTGACATGC | 1602 |
| 21560-25369 | 1446 | TCAAACTGGTCTAAATTGCTACTACCCACTTTATAGATATGGATTTTACC | 1495 |
| | | .....\|\|.\|\|.\|..\|.\|....\|\|.\|\|.......\|.\|\|\|.....\|\|..... | |
| hub_1489433_G | 1603 | AGCTTCTTGTATTGAATATCCCTGCCTTTCCATTGGATGGCACTTAGCAG | 1652 |
| 21560-25369 | 1496 | CTAC--TGATGGTGTTG----GTCAC----CAACCTTATAGGGTAGTAGT | 1535 |
| | | \|.\|\| \|.\|..\|\|.\|\|\| .\|\|\|\| \|...\|\|\|\|\|...\|..\|.\|\|. | |
| hub_1489433_G | 1653 | CAACGTTCACTGTATTGTTTAATCACACCTCGTACTTATTCTGATGGAGA | 1702 |
| 21560-25369 | 1536 | ACTTT----CTTTTGAACTTCTAAATGCACCAGCAACTGTTTGTGGACCT | 1581 |
| | | \|.\|\|\| \|..\|\|\|\|.\|. \|....\|.\|.\|...\|\|.\|..\|\|\|.\|...\|. | |
| hub_1489433_G | 1703 | AATTTTTGTCAGTTGAGCA-CACTTTCCTCTCTCATCCTTTTATTTTCT- | 1750 |
| 21560-25369 | 1582 | AAGAAGTCTACTAACTTGGTTAAAAATAAATGTG-TCAAT-TTCAACTTT | 1629 |
| | | ..\|\|\|\|\| ...\|\|.\|\|\|....\|\|.\|..\|.\| .\|\|\|. \|.\|.\|.\|.\| | |
| hub_1489433_G | 1751 | ---GTGTCTA--GCTTTAGTTTGGGATGAGGGAGGACAAAGTACTATTAT | 1795 |
| 21560-25369 | 1630 | AATGGTTTAAC--TGGCACAGGTGTCCTCACAGAGTCTAATAAAAAGTTT | 1677 |
| | | .\|\|\|...\|.\|\| \|\|\|\|.\|.\|\|.\|.\|\|\|\|.\|\|.\|..\|\|.\|..\|..\|.... | |
| hub_1489433_G | 1796 | TATGAAATTACAGTGGCTCTGGAGGCCTCTCAAATCCTGACTATGACACA | 1845 |
| 21560-25369 | 1678 | CTACCTTTCCAACAATTTGGTAGAGACATTGCAGACACTACTGAT--GCC | 1725 |
| | | ..\|..\|\|...\|\|..\|.\|\|...\|\|....\|.\|.\|.........\|\|.\| \|\|. | |
| hub_1489433_G | 1846 | GAAAATTCTGAAATAATTCACAGCAGGAGTACTATAGGACTTGGTCAGCT | 1895 |
| 21560-25369 | 1726 | GTCCGTGATCCACAGACACTTGAGATTCTTGACATTACACCATGTTCTTT | 1775 |
| | | .\|.\|.\|....\|\|.\|....\|.......\|.\|\|\|.\|..\|.\|......\|\|\|\|.. | |
| hub_1489433_G | 1896 | TTGCATTGAACATAACTCCACATCTATATTGGCTCTGCTTTTGCTTCTCA | 1945 |
| 21560-25369 | 1776 | TGGTGGTGT-CAGTGTTATAACA---CCTGGAACAAATGCCTCTAACCAG | 1821 |
| | | ...\|....\| \|.....\|\|\|..\|\| \|..\|\|.\|\|.\|.\|...\|.\|\|.\|\|.. | |
| hub_1489433_G | 1946 | ATATTAAATGCTCAAATATGTCAGTGCTAGGCACTATTATTTATATCCCT | 1995 |
| 21560-25369 | 1822 | GTTGCTGTTCTTTATCAGGATGTTAACTGCA--CAGAAGTCCCTGTTGCT | 1869 |
| | | .\|......\|.\|\|\|. ....\|.\|....\|.\|\|\| \|\|\|\|\|\|.\|.\|.\|\| \|. | |
| hub_1489433_G | 1996 | CTGAAACATGTTTCTATTCAAGGATGCAGCATTCAGAAGACTCAGT--CC | 2043 |
| 21560-25369 | 1870 | ATCCATGCAGACCAACTTACTCCCACTTGGCGTGTTTACTCCACAGGTTC | 1919 |
| | | \|.\|.\|...\|.\|..\|\|...\|\|\|.\|\| \|\|\|\|\|..\|.\|.\|\|.. \|\|.\|\|. | |
| hub_1489433_G | 2044 | AGCGAGTGACAGAAAAAGACTTCC-CTTGGATTATCTATG----AGATTG | 2088 |
| 21560-25369 | 1920 | TAATGTTTTTCAAACACGTGCAGGTTGTTTAATAGGGGCT-GAACATGTC | 1968 |
| | | \|\|\|\|...\|\|.....\|\|..\| \|.\|.\|...\|.\|\|\|\|..\|.\|\| \|\|.\|\|\|... | |
| hub_1489433_G | 2089 | TAATAGCTTATCTGCATAT-CTGCTCACTGAATACTGCCTCGATCATTCA | 2137 |
| 21560-25369 | 1969 | AATAACTCG-TATGAGTGTGACATACCTATTGGTGC-AGGAATATGCGCC | 2016 |
| | | .\|\|\|.\|\|.\| \|...\|.\|\|.\|..\|\|...\|\|...\|\|\|. \|.\|\|\|\|...\|..\| | |
| hub_1489433_G | 2138 | TATATCTGGCTCACAATGGGTAATCAATAAATGTGTGATGAATGGTCTAC | 2187 |
| 21560-25369 | 2017 | AGTTATCAGA------CTCAAACTAATTCACGTAGTGTGGCCAGTCAAT- | 2059 |
| | | \|.\|\|..\|\|\|\| \|.\|.\|\|\|\|...\|\|\|.\|..\| \|...\|\|\|\|\|...\| | |
| hub_1489433_G | 2188 | AATTCCCAGATTGCAGCCCTAACTTGCTCATGATG-GCTTCCAGTAGTTT | 2236 |
| 21560-25369 | 2060 | -CTATTATTGCCTACACTATGTCACTTGGTGCAGAAAATTCAGTTGCTTA | 2108 |
| | | \|\|\|\|.\|..\|\|\| \|\|\|....\|\|\|\|.\| \|\|\|\|\|\|.\|.....\|\|\| \|... | |
| hub_1489433_G | 2237 | TCTATCAAAGCC-ACATGTGGTCAGT--GTGCAGGATGAGGAGT--CGAG | 2281 |
| 21560-25369 | 2109 | TTCTAATAACTCTATTGCCATACCTACAAATTTTACTATTAGTGTGACCA | 2158 |
| | | ..\|\|.\|.\|\|\|\|\|.\|.\| \|.\|.\|....\|.\|.\|....\|..\|\|\|.\|...\|\|.. | |
| hub_1489433_G | 2282 | CCCTTAAAACTCAACT-CTAGAAGACCTACTGAAGCAGTTATTACAACAT | 2330 |
| 21560-25369 | 2159 | -CTGAAATTCTACCTGT----GTCTATGACAA-AGACATCGGTAGACTGT | 2202 |
| | | \|\|..\|\|\|.\|.....\|. \|.\|\|...\|\|\|. \|\|\|.\|..\|\|\|.\|.\|\|\|. | |
| hub_1489433_G | 2331 | GCTACAATACACAAAGAACAAGACTTGTACATCAGAAACAGGTTGTCTGA | 2380 |
| 21560-25369 | 2203 | ACAATGTATAT-TTGTGGTGATTCAACTGAGTGCAGCAACCTTTTGTTG- | 2250 |
| | | \|.\|\|..\|.\|.\| \|\|\|.\|\|..\|\|..\|.\|..\|.\|\|...\|.\|..\|\|\|\|.\|.\| | |
| hub_1489433_G | 2381 | AAAAGTTTTCTATTGGGGGAATGAAGCAAATTGAGCCTAAGTTTTCTGGA | 2430 |
| 21560-25369 | 2251 | CAATATGGTA--GTTTTTGCACACAATTAAATCGTGCTTTAACTGGAATA | 2298 |
| | | \|\|\|.\|.\|..\| \|.\|..\|..\|\|.\|\|.\|\|.\|\| \|\| \|\|...\|\|...\|..\| | |
| hub_1489433_G | 2431 | CAAAAAGAAAAGGCTGATTTACTCAGTTTAA--GT-CTAAGACCAAAGAA | 2477 |
| 21560-25369 | 2299 | GCTGT-TGAACAGGACAAAAATACTCAAGAAGTTTT-TGCTCAAGTTAAA | 2346 |
| | | ...\|\| \|\|\|..\|..\|\|\|\|.\|.....\|..\|\|..\|\|.\| \|\|\|.\|....\|.\|. | |
| hub_1489433_G | 2478 | TAAGTCTGAGAAAAACAAGATGTTACCTGATCTTATATGCACTCTATTAT | 2527 |
| 21560-25369 | 2347 | CAAATTTATAAGACAC--CACCAATTAAAGATTTTGGTGGTTTCAAT-TT | 2393 |
| | | .\|..\|\|\|......\|\|. \|.\|...\|.\|.\|\|...\|\|\|\|\|..\|....\|\| .\| | |
| hub_1489433_G | 2528 | TATTTTTGCTTTGCATGTCCCTTGTAATAGTGATTGGTTTTAATGATCAT | 2577 |
| 21560-25369 | 2394 | TTCACA--AATATTACCAGATCCATCAAAACCAAGCAAGAGGTCATTTAT | 2441 |
| | | \|\|\|\|.\| \|\|.\|\|\|\|..\|.......\|\|......\|.\|.....\|\|\|\|...\|\| | |
| hub_1489433_G | 2578 | TTCATATAAAAATTAAAAAGAAGTACATTTTTTAACTTCCTGTCAAAAAT | 2627 |
| 21560-25369 | 2442 | TGAGGATTTACTTTTCAATAAAGTGACACTTGCT-GATGCTGGCTTCATC | 2490 |
| | | \|..\|..\|....\|..\|...\|.\|\|...\|\|\|\|..\|.\| \|.\|\|\|\|..\| \|.\|\| | |
| hub_1489433_G | 2628 | TCTGCCTAAGGTACTTCCTCAACACACACACGTTAGTTGCTACC--CCTC | 2675 |
| 21560-25369 | 2491 | AAACAATATGGTGATTGCCTTGGTGATATTGCTGCTAGGGATCTTATTTG | 2540 |
| | | ...\|\|\| \|\|...\|...\|.\|\|....\|.\|. \|\|.\|..\|....\|\|\|.\|\|\|\| | |
| hub_1489433_G | 2676 | CTTCAA---GGCTCTGTTCATGCCCGTCTC-CTCCACGAAGACTTTTTTG | 2721 |
| 21560-25369 | 2541 | TGCTCAAAAGTTCAATGGCCTTACTGTTCTGCCA----------CCTTTG | 2580 |
| | | \|.\|\|..\|......\|\|.\|..\|\|..\|\|..\|\|.\|.\|\| \|\|\|..\| | |
| hub_1489433_G | 2722 | TTCTACACCTAGAAAGGCTCTGCCTACTCAGGCAGTTGTTATTACCTCCG | 2771 |
| 21560-25369 | 2581 | CTCACAGATGAAATGATCGCTCAATACACTTCTGCACTATTAGCAGGTAC | 2630 |
| | | .\|..\|..\|..\|...\|\|\|\|.\|\|...\|\|...\|..\|....\|\|\|.\|..\|\|\|\|.. | |
| hub_1489433_G | 2772 | ATTTCCTACTATCAGATCTCTTCGTATTATCTTCTTATATGACTAGGTCT | 2821 |
| 21560-25369 | 2631 | AATCACTTCTGGTTGGACTTTTGGTGCAGGTGCTGCTTTACAAATACCAT | 2680 |
| | | .\|\|\|.\|..\|.......\|\|..\|...\|....\|.\|\|\|\|...\|\| ...\|.\|... | |
| hub_1489433_G | 2822 | CATCTCCCCCTCAACCACAATCTCTCTGAGGGCTGGAATA-TTGTGCACA | 2870 |
| 21560-25369 | 2681 | TTGCCATGCAAATGGCTTATAGGTTTAATGGTATTGGAGTTACACAGAAT | 2730 |
| | | \|\|\|\|\|.\|\|\|\|.\|\|.. \|.\|..\|.\|..\|..\|\|\|\|\|..\|..\|.\|....\|..\| | |
| hub_1489433_G | 2871 | TTGCCTTGCACATAA-TAAAGGCTCCAGAGGTATCTGTCTAAACTGGCTT | 2919 |
| 21560-25369 | 2731 | GTTCTCTATGAGA--ACCAAAAATTGAT---TGCCAACCAGTTTAATAGT | 2775 |
| | | ..\|.\|\|..\|\|\|\|\| \|\|.\|..\|.\|\|..\| \|\|\|\|\|..\|\|.\|\|\|.\|...\| | |
| hub_1489433_G | 2920 | TATTTCCTTGAGACTACAAGCACTTATTCTGTGCCAGGCACTTTTAGGTT | 2969 |
| 21560-25369 | 2776 | GCT-------ATTGGCAAAATTCAAGACTCACTTTCTTC--TACAGCAAG | 2816 |
| | | .\|. \|..\|\|.\|.\|\|..\|.\|\|\|\|.\|\|....\|\|.\| \|...\|.\|.. | |
| hub_1489433_G | 2970 | CCAGGGAAAAAGAGGTACAAAACCAGACACAAACCCTACCGTTATGGAGC | 3019 |
| 21560-25369 | 2817 | TGCACTTGGAAAACTTCAAGATGTTG---TCAACCAAAAT--GCACAAGC | 2861 |
| | | \|...\|\|\|...\|\|.....\|.\|..\|..\| \|.\|\|\|\|....\| \|..\|...\| | |
| hub_1489433_G | 3020 | TTACCTTTTTAATTAAAAGGTGGAAGGGATGAACCTTTTTTTGGTCTCTC | 3069 |
| 21560-25369 | 2862 | TTTAAACACGCT--TGTTAAACA----ACTTAGCTCCAATT--TTGGA-G | 2902 |
| | | \|..\|\|\|...\|\|. .\|...\|.\|\| \|..\|\|\|....\|\|.\| \|\|\|.\| \| | |
| hub_1489433_G | 3070 | TAGAAAGTTGCAGCAGGAGACCATAGGAAATAGTATAAAATAGTTGAAAG | 3119 |
| 21560-25369 | 2903 | CTATTTCTAGCGTGTTAAATGATATCCTT-TCACGTCTCGACAAAGTTGA | 2951 |
| | | \|..\|.\|..\|\|.\|\|\|....\|.\|\|\|\|.\|.\|. \|\|.\|.\|\|\|\|.\|....\|.\|\|. | |
| hub_1489433_G | 3120 | CACTGTGGAGTGTGAGTCAGGATACCTTGGTCTCATCTCTAATTTGATGT | 3169 |
| 21560-25369 | 2952 | GGCTGAAGTGCAGATTGACAGGTTGATCACAGGCAGACTTCAAAGCTTGC | 3001 |
| | | ..\|\| .\|.\|\|\|.\|\|\|.. \|\|.\|.\|\|..\|\|....\|\|......\|\|... | |
| hub_1489433_G | 3170 | ATCT--TGAGCACATTTC----TTAAACATTGGTCATCTGTTTCCCTGTA | 3213 |
| 21560-25369 | 3002 | AGACATATGTGACTCAACAATTAATTAGAGCTGCAGAAATCAGAG--CTT | 3049 |
| | | .\|.\|\|\|\|\|..\|\|.\|\|\|.....\|.\|.\|.\|.....\|.\|\|\| \|\|\|\|\|. \|.. | |
| hub_1489433_G | 3214 | TGCCATATAGGAATCATATGGTTACTGGGAAAACTGAA-TCAGAAAACAG | 3262 |
| 21560-25369 | 3050 | CTGCCAATCTTGCTG-------------CTACTAAAATGTCAGAGTGTGT | 3086 |
| | | .\|\|\|.\|\|\|\|.\|\|.\|\| \|.\|\|...\|.....\|\|..\|.\|.\| | |
| hub_1489433_G | 3263 | ATGCAAATCATGTTGGAGGGAACTTTCTCAACCTGATAAAAAGCATCTAT | 3312 |
| 21560-25369 | 3087 | ACTCGGACAATCAAAAAGAGTTGATTTTTGTGGAAAAGGCTATCATCTTA | 3136 |
| | | .......\|.\|.....\|\|.\|....\|.\|\|....\|..\|\|\|\|.\|\|...\|.\|.\|. | |
| hub_1489433_G | 3313 | GAAAAACCCACAGCTAACACCATACTTAAAGGTGAAAGACTGGAAGCCTT | 3362 |
| 21560-25369 | 3137 | TGTCTTTCCCTCAGTCAGCACCTCATGGTGTAGTCTTCTTGCATGTGACA | 3186 |
| | | ..\|\|......\|\|\|\|\|.\| \|\|...\|\|.\|\|.....\|..\|\|\|..\|...\|\|..\| | |
| hub_1489433_G | 3363 | CTTCCGAAGATCAGTAA-CAAGACAAGGATGTCTGCTCTCACCACTGCTA | 3411 |
| 21560-25369 | 3187 | TATGTCCCTGCACAAGAAAAGAACTTCACAACTGCTCCTGCCATTTGTCA | 3236 |
| | | \|....\|..\|..\|\|..\|\| \|\|..\|\|...\|\|..\|.\|\|...........\|.\| | |
| hub_1489433_G | 3412 | TTCAACATTCTACCGGA--AGTTCTAGCCAGGTTCTAAGTAAGAAAATGA | 3459 |
| 21560-25369 | 3237 | TGATGGAAAAGCACACTTTCCACGTGAAGGTGTTTTCG-- TTTCAAATG | 3283 |
| | | ......\|\|.\|....\|..\|\|..\|..\|\|\|\|\|..\|\|..\|.. \|.\|\|.\|.\|. | |
| hub_1489433_G | 3460 | AATAAAAAGAATCAAGATTGGAAATGAAGAAGTACTAAAACTATCTATTT | 3509 |
| 21560-25369 | 3284 | GCACACACTGGTTTGTTACACAAAGGAATTTTTATGAACCACAAATTATT | 3333 |
| | | .\|\|.\|..........\|\|\|\|..\|.\|..\|...\|..\|..\|.\|\|\|\|.....\|.. | |
| hub_1489433_G | 3510 | TCATATGACATGACCTTACTTAGAAAATGCTAAAGAATCCACCCCCAACC | 3559 |
| 21560-25369 | 3334 | ACAACA--GACAACACATTTGTCTCTGGTAGCTGTGAT----GTTGTAAT | 3377 |
| | | .\|.\|\|. .\|\|\|\|.\|\|..\|\|....\|\|..\|\|..\|\|...\| ....\|... | |
| hub_1489433_G | 3560 | CCCACCCCAACAAAACTATTAAAGCTAATAAGTGAATTCAGCAAGATTTC | 3609 |
| 21560-25369 | 3378 | AGGAATTGTCAACAACACAGTTTATGATCCTTTGCAACCAGAACTTGATT | 3427 |
| | | \|\|\|\|........\|\|\|.\|\|.\|...\|..\|....\|\|\|.\|. ..\|\|..\|.. | |
| hub_1489433_G | 3610 | AGGATACAAGGTCAATACGGAAAAAAAAAAGTTGTAT----TTCTATAAA | 3655 |
| 21560-25369 | 3428 | CATTCAAGGAGGAGTTGGATAAATACTTTAAAAATCATACATCACCTGAT | 3477 |
| | | \|...\|\|\|.\|\|..\|.\|..\|\|.\|\|..\|..\|\|\|\|\|\|\|.\|\| \|\|\|.\|\|..\|... | |
| hub_1489433_G | 3656 | CTAACAATGAACAATCTGAAAATGAAATTAAAAAACA-ACACCATTTATG | 3704 |
| 21560-25369 | 3478 | GTAGATTTAGGTGACATTTCTGGCATTAATGCTTCAGT----TGTCAATA | 3523 |
| | | .\|\|\|..\|\|\|......\|.\|\|..\|\|.\|\|.\|\|\|....\|\|......\|\|\|.\|... | |
| hub_1489433_G | 3705 | ATAGCATTAAAAAGAAATTAAGGAATAAATTTAGCAAAGAAGTGTAACAC | 3754 |
| 21560-25369 | 3524 | TTCAAAAGGAAATTGACCGCCTCAATG----AGGTTGCCAAAAATCTAAA | 3569 |
| | | \|\|..\|...\|.\|\|...\|.\|....\|\|.\|\| \|.\|....\|\|\|\|.\|.\|\|\|\|\| | |
| hub_1489433_G | 3755 | TTGTACGTGGAAAACAACAAAACATTGTTGAAAGAAATCAAAGACCTAAA | 3804 |
| 21560-25369 | 3570 | TGAATCTCTCATCGATCT-CCAAGAACTTGGAAAGTATGAACAGTATATA | 3618 |
| | | \|.\|\|..\|.\|.\|.....\|\| \|\|..........\|.\|..\|...\|...\|...\|\| | |
| hub_1489433_G | 3805 | TAAAATTTTTAAAATCCTGCCTTTGTGGATTAGAACACTTAATTTTGTTA | 3854 |
| 21560-25369 | 3619 | AAATGGCCATGGTACATTTGGCTAGGTTTTATAGCTGGCTTGATTGCCAT | 3668 |
| | | \|\|\|\|.\|\|..\|..\|.\| \|....\|.\|..\|\|..\|..\|.\|.......\|.\|.\|. | |
| hub_1489433_G | 3855 | AAATAGCAGTACTCC--TCAATTTGAATTATTCACAGCAAATCCTACAAA | 3902 |
| 21560-25369 | 3669 | AATAATGGTCACGATTATGCTT-TGCTGTA--TGACCAGTTGC-TGCAGT | 3714 |
| | | \|\|\|..\|.\|..\|\|..\|\|\|\|..\|. \|\|\|.\|.\| \|\|\|\|.\|\|.\|\|. \|..\|.. | |
| hub_1489433_G | 3903 | AATCTTAGCTACCTTTATTTTCCTGCAGAAATTGACAAGCTGAGTTTAAA | 3952 |
| 21560-25369 | 3715 | TGTCTCAAGG----GCTGTTGTTCTTGCGGATCTTGCTGCAAATTTGATG | 3760 |
| | | \|.\|..\|\|.\|\| \|\|.......\|..\|...\|\|\|..... \|\|\|..\|\|\|\|.. | |
| hub_1489433_G | 3953 | TTTTACATGGAAATGCAAGGAACCCAGAATATCCAAAA-CAATCTTGAAA | 4001 |
| 21560-25369 | 3761 | AAGACGA-CTCTGAGCCAGTGCTCAAAGGAGTCAAATTACATTACACA | 3807 |
| | | \|\|.\|.\|\| \|...\|.\|..\|...\|\|\|\|.\|. ...\|.\|\|\|\|..\|..\|\|..\| | |
| hub_1489433_G | 4002 | AAAAGGAACAAAGTGGGAAGACTCATAC-TTCCTAATTTAAAAACTGA | 4048 |

To investigate the nature of the viruses identified by Kraken2 systematically in detail, pipelines that integrate these sequencing reads to identify viral-like sequences with high confidence were developed (FIG. 9A). First, a metagenomic classification method (Kraken2) was employed to detect possible viral sequences. Next, a two-pronged strategy for assembling the RNA-seq into transcripts that can be utilized for viral sequence analysis was used. The first strategy was bottom-up: a de novo assembly (using 4,707,164 of the total) of the RNA-seq reads was performed that classified them as viruses and separated them into putative mammalian or non-mammalian viruses based on the VIRION database and then verified that the respective transcripts map to the bat genome. Additionally, 5 kb flanks per transcript locus within the genome were extracted to determine the extent of each potential viral integration. Using the bat genome as a scaffold, the second method was a “top-down” approach and involved mapping the Kraken2 codified RNA-seq reads to the bat genome and then extracting the respective genomic sequences with or without adding 5 kb flanking regions on each side. Then BLAST was utilized against a mammalian and a non-mammalian virus database to discover viral hits. Importantly, to avoid viral matches by chance, all transcripts or genomic sequences to each database were mapped after randomizing them by dinucleotide shuffling.

When the pipelines were applied to the bat stem cell transcriptome data, 311 and 82 transcripts estimated to be mammalian viruses and 351 and 58 non-mammalian viruses (bottom-up and top-down, respectively) were obtained. Direct genome mapping yielded 56 hits (out of 63 transcripts, bottom-up; 25 unique) and 82 (all transcripts from top-down approach; 19 unique) mammalian virus hits against the R. ferrumequinum genome. After applying the BLAST threshold, 31 transcripts, with 13 transcripts shared between both methods, mapped to both a viral sequence and a locus in the bat genome. The BLAST step on extended sequences from both methods yielded a total of 16 sequences within the R. ferrumequinum genome that aligned with known viruses at high confidence. Validating this stringent approach, using the shuffled sequence data, no hits were found for the bottom-up sequences and only two top-down BLAST hits passed the threshold, indicating that the vast majority of the viral hits are not chance matches but reflect bona fide homology. Indeed, this was confirmed by manual inspection of the alignment hits, which showed numerous longer, well-aligning regions substantially exceeding the length and quality of the matches of randomized sequences. The results indicated a taxonomically diverse collection of attributed viruses from a number of major viral families. Included among them are Flaviviridae, Herpesviridae, Poxviridae and Retroviridae. Overall, this exhaustive analysis shows that bat stem cells contain a surprising diversity of sequences that resemble viral genomes. To implement an orthogonal metagenomic strategy, a direct alignment method using the Microsoft Research Premonition pipeline was employed. Using bat stem cell RNA-seq reads as input, this classifier positively recognized 419 different putative viral-like sequences. Again, the taxonomy included a number of important viral families, such as Paramyxoviridae, Flaviviridae, Retroviridae, Coronaviridae and Poxviridae. Manual examination of the expressed virus-sequence revealed a wide range of lengths ranging from (near) full-length viral sequences to specific viral protein encoding domains to short fragments of viral regulatory sequences. As before, the Premonition pipeline predicted sequences were mapped to the bat genome, extended 5000 bp flanks, and performed BLAST searches against the VirusDB and shoed that a total of 13 extended bat genome sequences mapped to know virus genomes, 9 of which overlapped with the bottom-up/top-down approaches, indicating a high degree of consistency. Viruses linked to Hardy-Zuckermann 4 feline sarcoma virus, Friend murine leukemia virus, Porcine endogenous retrovirus E, and PreXMRV-1 provirus were examples. Consequently, both metagenomics pipelines methods reveal a significant number of endogenized sequences that resemble viral genomes with a final count of 20 high-confidence viral hits across all methods. Exemplary sequences of possible viral origin discovered with this method are listed in SEQ ID NOs: 1-349.

### Example 11 Identification of Viral Proteins Useful in Vaccine Development

This example describes the identification of viral nucleic acid sequences and viral proteins present in the bat genome and in bat cells for the use in vaccine development.

Briefly, viral DNA and RNA sequences can be identified as described in Example 8 Example 9, and Example 10. The viral DNA or RNA sequences can be assembled into long contigs such as SEQ ID NO: 1-349. The contigs can be translated into amino acid sequences. The identified amino acid sequences can be compared to known nucleic acid sequences and proteins using methods like BLAST (www.web.expasy.org/blast) and the sequences can be aligned and translated into amino acid sequences of peptides and proteins. Vital viral enzymes such as the essential genes are replicase ORF1ab, spike (S), envelope (E), membrane (M) and nucleocapsid (N), RNA polymerases, kinases, and viral proteases can be identified using homology models and sequence alignment as described in Example 10.

In order to develop a vaccine, immunogenic CD8+ T cell epitopes in the identified vital virus proteins can be predicted using for example a machine learning platform such as described in Bulik-Sullivan et al. (2018) Deep learning using tumor HLA peptide mass spectrometry datasets improves neoantigen identification. Nature Biotechnology 2018, 37(1). Predictions for these epitopes can be run for each HLA class I allele. Candidate CD8+ epitopes can be maximized for coverage of the prevalent HLA-types in a given population. The method described for generating candidate CD8/MHC class I epitopes can be used to generate peptides with sizes between 9 and 20 amino acids. Further, potential HLA-DRB, HLA-DQ, and HLA-DP MHC class II epitopes can be predicted. The predicted epitopes can then be displayed by MHCs and recognized by human T cells can be tested with methods such as mass spectrometry based HLA I and HLA II epitope binding prediction tools (e.g., Immune Epitope Database and Analysis Resource, www.iedb.org). Epitopes such as for HLA-I or HLA-II can be scored and identified for peptide sequences derived from the identified vital viral enzyme. Top-ranking peptides can be prioritized based on expected population coverage (allele frequencies). Predicted peptides can be tested for T cell responses using PBMCs from human donors and MHC multimers loaded with peptides and ranked. Further assays of T cell reactivity (e.g., interferon-gamma ELISpots, tetramers), which are stricter measures for T cell immunogenicity to epitopes, can be performed to further identify top immunogenic peptides.

The nucleotide sequences for the identified epitopes and peptides can be cloned into vectors with expression cassettes in order to express viral proteins for use in vaccines in recombinant cell. Recombinant cells for example HEK cells or CHO cells can be transfected with these vectors to produce vaccines, such as adenovirus based vaccines. mRNA based vaccines can be synthesized chemically or enzymatically and packaged into lipid particles, nanoparticles or liposomes for further delivery to a subject.

### REFERENCES

Anders S, Huber W. Differential expression analysis for sequence count data. Genome Biol. 2010; 11(10):R106. doi: 10.1186/gb-2010-11-10-r106. Epub 2010 Oct. 27. PMID: 20979621; PMCID: PMC3218662.
Andrews, S. (2010). FastQC: a quality control tool for high throughput sequence data. Available online at: http://www.bioinformatics.bbsrc.ac.uk/projects/fastqc.
Bolger A M, Lohse M, Usadel B. Trimmomatic: a flexible trimmer for Illumina sequence data. Bioinformatics. 2014 Aug. 1; 30(15):2114-20. doi: 10.1093/bioinformatics/btu170. Epub 2014 Apr. 1. PMID: 24695404; PMCID: PMC4103590.
Carlson C J, Gibb R J, Albery G F, Brierley L, Connor R P, Dallas T A, Eskew E A, Fagre A C, Farrell M J, Frank H K, Muylaert R L, Poisot T, Rasmussen A L, Ryan S J, Seifert S N. The Global Virome in One Network (VIRION): an Atlas of Vertebrate-Virus Associations. mBio. 2022 Apr. 26; 13(2):e0298521. doi: 10.1128/mbio.02985-21. Epub 2022 Mar. 1. PMID: 35229639; PMCID: PMC8941870.
Carter A C, Davis-Dusenbery B N, Koszka K, Ichida J K, Eggan K. Nanog-independent reprogramming to iPSCs with canonical factors. Stem Cell Reports. 2014 Jan. 31; 2(2):119-26. doi: 10.1016/j.stemcr.2013.12.010. PMID: 24527385; PMCID: PMC3923195.
Dejosez M, Krumenacker J S, Zitur L J, Passeri M, Chu L F, Songyang Z, Thomson J A, Zwaka T P. Ronin is essential for embryogenesis and the pluripotency of mouse embryonic stem cells. Cell. 2008 Jun. 27; 133(7):1162-74. doi: 10.1016/j.cell.2008.05.047.
Ewels P, Magnusson M, Lundin S, Kaller M. MultiQC: summarize analysis results for multiple tools and samples in a single report. Bioinformatics. 2016 Oct. 1; 32(19):3047-8. doi: 10.1093/bioinformatics/btw354. Epub 2016 Jun. 16. PMID: 27312411; PMCID: PMC5039924.
Huang Z, Whelan C V, Foley N M, Jebb D, Touzalin F, Petit E J, Puechmaille S J, Teeling E C. Longitudinal comparative transcriptomics reveals unique mechanisms underlying extended healthspan in bats. Nat Ecol Evol. 2019 July; 3(7):1110-1120. doi: 10.1038/s41559-019-0913-3. Epub 2019 Jun. 10. PMID: 31182815.
Jebb D, Huang Z, Pippel M, Hughes G M, Lavrichenko K, Devanna P, Winkler S, Jermiin L S, Skirmuntt E C, Katzourakis A, Burkitt-Gray L, Ray DA, Sullivan K A M, Roscito J G, Kirilenko B M, Divalos L M, Corthals A P, Power M L, Jones G, Ransome R D, Dechmann D K N, Locatelli A G, Puechmaille S J, Fedrigo O, Jarvis E D, Hiller M, Vernes S C, Myers E W, Teeling E C. Six reference-quality genomes reveal evolution of bat adaptations. Nature. 2020 July; 583(7817):578-584. doi: 10.1038/s41586-020-2486-3. Epub 2020 Jul. 22. PMID: 32699395; PMCID: PMC8075899.
Kacprzyk J, Locatelli A G, Hughes G M, Huang Z, Clarke M, Gorbunova V, Sacchi C, Stewart G S, Teeling E C. Evolution of mammalian longevity: age-related increase in autophagy in bats compared to other mammals. Aging (Albany NY). 2021 Mar. 21; 13(6):7998-8025. doi: 10.18632/aging.202852. Epub 2021 Mar. 21. PMID: 33744862; PMCID: PMC8034928.
Kim D, Paggi J M, Park C, Bennett C, Salzberg S L. Graph-based genome alignment and genotyping with HISAT2 and HISAT-genotype. Nat Biotechnol. 2019 August; 37(8):907-915. doi: 10.1038/s41587-019-0201-4. Epub 2019 Aug. 2. PMID: 31375807; PMCID: PMC7605509.
Knaupp A S, Buckberry S, Pflueger J, Lim S M, Ford E, Larcombe M R, Rossello F J, de Mendoza A, Alaei S, Firas J, Holmes M L, Nair S S, Clark S J, Nefzger C M, Lister R, Polo J M. Transient and Permanent Reconfiguration of Chromatin and Transcription Factor Occupancy Drive Reprogramming. Cell Stem Cell. 2017 Dec. 7; 21(6):834-845.e6. doi: 10.1016/j.stem.2017.11.007. PMID: 29220667.
Krueger, F. (2012). A wrapper tool around Cutadapt and FastQC to consistently apply quality and adapter trimming to FastQ files, with some extra functionality for MspI-digested RRBS-type (Reduced Representation Bisulfite-Seq) libraries. Available online at: https://www.bioinformatics.babraham.ac.uk/projects/trim_galore/
Li H, Handsaker B, Wysoker A, Fennell T, Ruan J, Homer N, Marth G, Abecasis G, Durbin R; 1000 Genome Project Data Processing Subgroup. The Sequence Alignment/Map format and SAMtools. Bioinformatics. 2009 Aug. 15; 25(16):2078-9. doi: 10.1093/bioinformatics/btp352. Epub 2009 Jun. 8. PMID: 19505943; PMCID: PMC2723002.
Liao Y, Smyth G K, Shi W. featureCounts: an efficient general purpose program for assigning sequence reads to genomic features. Bioinformatics. 2014 Apr. 1; 30(7):923-30. doi: 10.1093/bioinformatics/btt656. Epub 2013 Nov. 13. PMID: 24227677.
Love M I, Huber W, Anders S. Moderated estimation of fold change and dispersion for RNA-seq data with DESeq2. Genome Biol. 2014; 15(12):550. doi: 10.1186/s13059-014-0550-8. PMID: 25516281; PMCID: PMC4302049.
Metsalu T, Vilo J. ClustVis: a web tool for visualizing clustering of multivariate data using Principal Component Analysis and heatmap. Nucleic Acids Res. 2015 Jul. 1; 43(W1):W566-70. doi: 10.1093/nar/gkv468. Epub 2015 May 12. PMID: 25969447; PMCID: PMC4489295.
Ramirez F, Ryan D P, Grining B, Bhardwaj V, Kilpert F, Richter A S, Heyne S, Dindar F, Manke T. deepTools2: a next generation web server for deep-sequencing data analysis. Nucleic Acids Res. 2016 Jul. 8; 44(W1):W160-5. doi: 10.1093/nar/gkw257. Epub 2016 Apr. 13. PMID: 27079975; PMCID: PMC4987876.
Robinson J T, Thorvaldsdóttir H, Winckler W, Guttman M, Lander E S, Getz G, Mesirov J P. Integrative genomics viewer. Nat Biotechnol. 2011 January; 29(1):24-6. doi: 10.1038/nbt.1754. PMID: 21221095; PMCID: PMC3346182.
Shannon P, Markiel A, Ozier O, Baliga N S, Wang J T, Ramage D, Amin N, Schwikowski B, Ideker T. Cytoscape: a software environment for integrated models of biomolecular interaction networks. Genome Res. 2003 November; 13(11):2498-504. doi: 10.1101/gr.1239303. PMID: 14597658; PMCID: PMC403769.
Wickham H (2016). ggplot2: Elegant Graphics for Data Analysis. Springer-Verlag New York. ISBN 978-3-319-24277-4; Available online at: https://ggplot2.tidyverse.org.
Wood D E, Lu J, Langmead B. Improved metagenomic analysis with Kraken 2. Genome Biol. 2019 Nov. 28; 20(1):257. doi: 10.1186/s13059-019-1891-0. PMID: 31779668; PMCID: PMC6883579.
Yang Z. PAML 4: phylogenetic analysis by maximum likelihood. Mol Biol Evol. 2007 August; 24(8):1586-91. doi: 10.1093/molbev/msm088. Epub 2007 May 4. PMID: 17483113.
Yoshimatsu S, Nakajima M, Iguchi A, Sanosaka T, Sato T, Nakamura M, Nakajima R, Arai E, Ishikawa M, Imaizumi K, Watanabe H, Okahara J, Noce T, Takeda Y, Sasaki E, Behr R, Edamura K, Shiozawa S, Okano H. Non-viral Induction of Transgene-free iPSCs from Somatic Fibroblasts of Multiple Mammalian Species. Stem Cell Reports. 2021 Apr. 13; 16(4):754-770. doi: 10.1016/j.stemcr.2021.03.002. Epub 2021 Apr. 1. PMID: 33798453; PMCID: PMC8072067.
Xie Z, Bailey A, Kuleshov M V, Clarke D J B, Evangelista J E, Jenkins S L, Lachmann A, Wojciechowicz M L, Kropiwnicki E, Jagodnik K M, Jeon M, Ma'ayan A. Gene Set Knowledge Discovery with Enrichr. Curr Protoc. 2021 March; 1(3):e90. doi: 10.1002/cpz1.90. PMID: 33780170; PMCID: PMC8152575.
Zhang Y, Liu T, Meyer C A, Eeckhoute J, Johnson D S, Bernstein B E, Nusbaum C, Myers R M, Brown M, Li W, Liu X S. Model-based analysis of ChIP-Seq (MACS). Genome Biol. 2008; 9(9):R137. doi: 10.1186/gb-2008-9-9-r137. Epub 2008 Sep. 17. PMID: 18798982; PMCID: PMC2592715.

### EQUIVALENTS/OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

### SEQUENCE LISTING

| SEQ | |
|---|---|
| ID | |
| NO: | Sequence |
| 1 | RFe-V-MD1 |
| | GGAGAGAATTGATCAGAACTCCTGTCTTGTCTCCGGTCTTTGTGTCTCCCATTTTCCTCCCTTCTAGGTG |
| | CTTCGGGGTCCCTCGTGTAGTGTCCCGCGGGTCGGGACAACTGGCGCCCAACGTGGGGCCTGAAGTCTCC |
| | TAGAAGACGAGACGCCTGAGTTCGTCCGGTCTAAGGAGCTGCAGCATATTTCTTCTTTGATCACCATAAG |
| | ACTACCCAACTTGTGGGAGATCTGTACAGGTAAGCGGACGACTCCTTCAAAAAAATGGGACATATATTTG |
| | TGTTCTAGACTTATGTATAGTCTACAGGCTTCCCCTCAGACACTTAGACTAGGGTTCCCCTAACCTGTTG |
| | TCCCAGTCTCCCTTTTTATCTGCTCTCAGCTCACTTTGGGTTTTAGTCGTTCACCAACGAGACAGTTTTC |
| | TAGGTGTTTGGGACCGTTTGAGCGAGATTTTGCCTGCTTACTTTGAGCTCCAATCGTCCACCCAGAGGAT |
| | TTCCCGACCGGTTGAGTCCCGACTGGCTTTCGCCTGAGGGTCGTTACCAGCCGCGTCGCCTCTCGGGATC |
| | CGTGTTGGCGGATTATACCAACCGATTGCTCACGTAAGGGCTTTTTCTCCTCTCACCCCAACACCCCCGT |
| | GGCTCCGGCCGGGTGAGTCCCAAAAGACATTCGTCTGCGGGTCGTTACCATCCGTGCCGTCTCGTTTGGG |
| | TCCATGTTGGTGGATTGTACCAACCGACTGCCTATGTGAGGAGAGTCTTTATTCCTTATCATAATGGGAC |
| | AAGAGGTTAGTGTTCATGACATGTTTATCTCAGGACTAAAAGAGTCCTTACAAATAAGGAGAGTTAAAGT |
| | CAAGAAAAAAGATTTAGTTATCTTTTTTAATTTCTTAAAAGATGTTTGCCCTTGGCTCCCTCAGGAAGGA |
| | ACCATAGACCACAAAAGATGGAAAAGGATCGGAGATGCCCTTAATGACTTTTATAAAACTTTTGGCCCTA |
| | AAAAGAATCCCCATCACTGCTTTCACTTATTGGAATTGCATTATTGAGCTACTTATGGTACATCGCTACA |
| | CCCCTGACATCGACCGAGTGATACAAGAAGGAAACACATTTTTACAAAACGCTTCCCGCCCCTCCTCCTC |
| | CTTACAGGTCCCCTCTTCTAAGTCCTCTCACGATTCAGATTCTATTTCTATTTCAATGCCTCCTGAAGAT |
| | CCTGAGACCACCAAAAAAGATCCTAGTAAGCCTTATATCCTCCCCTACCACCTAATTGTCCTGATCTTAA |
| | TGTAAATTCTAGCCCACCTGAGGACGATCAGTTAAGCCCTGAGGACGAGGCTGATTTAGAGGAAGCTGCC |
| | GCTAAATATCATAATCCTGTCTGGCAGTTTCTGGCCTCTAATCAATTGCCCCCTCCCTATAATCCCCAAA |
| | TGCCTTTAGCTCCTATCCACGATCCTGATCAAACTCTCCTCTCCCACCAAGTCCAACAATTACAAAGAAC |
| | TGTTCAACTCAAAAAACAACATCTAACTCTCCTTAAACAACTTCAACAATTAGATTTACAACTCTCCTCT |
| | GCTGCTACTCAAAAAATTCCCCCCCCTTTCCATAAATCCTACAAAAACATTTCCCATCTCAAATAAAAAA |
| | AACCCTATTAATCTTTTCCCCGTTATTGAATTCCCCCCCAATAAAAACTGAAGGAGGCAGTGCAGATAGT |
| | GATAAAGACCCCGACAGAGACAATATAGAACCCCGCAAGACACTATAAACGCCTTGACTTAAAAACCACA |
| | AAAGAACTCAAAAAAGCGGTGGACGAATATGGCCCCACGGCCCCCTTTACACTCTCAATTTTACAATCCC |
| | TAGATGACCTCTGGTTAACCACCCATGATTGGCACTATTTGGCCCATGCCACCCTATCGGGGGGCGATTA |
| | TGTTCTCTGGAAATCTGAGTTTTCTGAGGCCTGTAAAGAAACTGCACACCGCAACGCAGAAGCGGGAGGC |
| | GAGTGCACTGATTCGACCTATGATAAGTTCAGGGGCTTTAAGCCCTACGATACAAATGAAGCTCAACTAC |
| | AATATCCATCTGGCCTTTTTTCTCAAATTTCACCTTGCCGCTACTAAGGCATGGAAAAAACTTCTCCCTA |
| | AGGGGCCGGCCACAACTCAACTCACTAGTATTAGACAGAGGCCAGAGGAACCTTATGCTGACTTCATCAG |
| | TCGCCTAACCAATGCCACTGAAAGACTCCTTGGTAGCACAGAAACTGATAGTGATTTTTTCAAACAATTA |
| | GCTTTTGAAAATACCAATTCTGCCTGTCAGGCAGCCATCTGCCCTAGAAAAAAGGATTCACTCTCTGATT |
| | ACATTCGCCTATGCACTGATATTTGGTCCTGGTCACCAAATGGGCCTCGCTATCGGGGCAGCTTTAAAAG |
| | ATTCATTACTTAATCTGTCTAAAGGCAAAAACAATTGTTTTTCATGTGGCCAGCCCGGACATTTCGCCAA |
| | ACAATGCCCAACCCCTCGCCAGAACACCATTAGGCCAACCCACTCCCACACCCATATTGCCCCCGCGAGT |
| | ATGTCCCAGATGCAAGAGAGACAAACATTGGGCCAATCAATGTAGATCAAAAATAGATGCCCACAACAAT |
| | CCTCTCCTGCCCCAGCAGGGGAAACTTCCTGAGGGGCCAGCCCCAGGCCCCTACAGGAGAATCCAAACCT |
| | TGGGGCGACTCGGTTTGCTCATCCACAACAAAACTTTGTCCCATCTCAAGTCTCCTCCGAGCAACCCCTG |
| | GCAGTGCTGGACTGGACCTCAGTCCCCTCCTCCAAATCAATATTAACTCCCTGACATGGGACCTCAGATA |
| | CTACCTACGGGTGTCACCGGACCCCTACCAACCAACACTTTTGGTCTAAAATTGGAAGAGGTAGTTCGAG |
| | CCTACAAGGCCTATATATTTACCCTGGTGTTATAGATAATGATTTTACGGGAGAAATACAGATTGTAGCC |
| | TCCTCCACTTCCTCTCTCATTTCTATACAACCGGGACAGAGAATAGCTCAACTACTCCTTCTCCCACTCC |
| | AGACCACCCATAAATCTGCCAACAATGAGCCTAGAAACAACAAAAATTTTAGATCCTCAGATGCTTATTG |
| | GATTCAAAATCTCTCCCCCAATAAGCCCATGCTAGATTTAAAACTTGATGGAAAAACCTTTTAAAGGCCT |
| | TATCGACACTGGTGCTGATGCAACCATTATTAGACAAAAAGACTGGCCGCTTTCTTGGCCCCTTTTCTGA |
| | CACACTTACTCACCTACAAGGCATAGGACAAACAACTAACCCCAGACAAAGTGCCAAGTTCCTAACATGG |
| | CTAGATAAAGAAAATAACTCTGGCACAGTACAACCTTACGTTGTACCCAACCCTCCCAGTAAATCTGTGG |
| | GGCCGTGACATATTATCCCAAATGGGAGTAATCATGTTCAGCCCCAATTCCAAGATAACCATCCAGATGT |
| | TAAAACAAGGGTTTCTCCCAGGTCAGGGATTAGAAAAACAAGGACAGGGAATTAAAAAACCCCTGTCTAC |
| | TGCTTCAGTGCCTGCCTTCGATTAGGCTTAGGACATTTTCACTAGTGGCCTCTGACCAACCTGCACCCCA |
| | TGCTGACCCTATATCCTGGAAAGGACAACTCGCCCATATGGGTGGATCAGTGGCCACTAAATTCAGAAAA |
| | ACTAAATGCTGCCAATCAGTTAGTGCAGAAACAATTGGCGGCAGGGCATCTAGAGCCCAGTAACTCCCCC |
| | CTGGAACACACCTATCTTTGTCGTAAAAAGAAATCTGGAAATTGGAGACTTCTCCAAGACCATAGGGAAG |
| | TCAATAAAACAATGATAATTATGGGCGCCCTTCAACCAGGCCTACCTACCCCCTGGAGCTATTCCCTCGG |
| | GGATCCTTAAAAATCATTATTGATCTCAAAGACTGCTTCTTCACTATCCCTCTACACCCTCAAGATAGAC |
| | AATGTTTTTGCTTTCAGCATACCTATAACTAATTTCCAAGGGCCCATGCAGAGATTTCAGTGGAAGGTCT |
| | TACCTCAGGGGCATGGCCAACAGCCCGACACTGTCAAATATTTGTTTGCTCTGGCCATCGATCCCATTCG |
| | AACTCAGTGGCCCTCTCTTTATATTATTCATTATATGGATGATATCTTAATAGCTGGCAAGAATGGGTCT |
| | GTACTTCCTCTCCCCAATATAAACAAGAAAAACCTCAGCCTTGTCCCGCTAAATGCTCTACTATTTACCC |
| | TATTATTCATAGTTCTTGTTACAATACCTATAAAACATGTACAGAAAAGATAACTCCTCTTATTATACGG |
| | CTGTCATGACAAGCACTGGTCCCGCTGTCCCTCATTCTGACTGGTCTAACACCCCTGCTGCGGTTGGCAT |
| | TTGGCTCCCATAAACCCGCACCCTGCGCGGCATCTAATATGTTAGAAAAAAATATTTGCTGGGCAGATCG |
| | AATCCCCTATACCATATGTTTCTGACGGCGGGGGGTCCAGCCGATCTCCAATCCAATGAAAAACGCATTA |
| | AAAAATTTGCTAAATACAAAAGACCCTTAACCCTAAATTTACCTATCACCCTTTGGCCCACCCTAAAAAA |
| | CCGGGGTCACGTGGACATTGATCCTCAGACTTTTGACATTCTTAGTTCTACCCACAAGTTATTGCTTTCT |
| | GTTAATTCATCCTACGCCAGAGACTGCTGGCTGTGTTTACTACAAGGTACCCCTTTACCATTAGCTATAC |
| | CCTATCCCTTTGTCACCTCTGACTACCAATAATTCATACAACATAGCTCTCCCCTTTTTTTAGTCCAACC |
| | CCTTGGCTTTAACAATACCCCGTGCATCCTCTCTCCCATTCAAAACAATACTACAGAGGTTATATTTAGG |
| | AAGCCTCTCCTTTACAAATTGCTCCTCCTTCATTAATGTATCCTCTCCTATGTGTACACCCAATGGATCG |
| | GTATATATTTGTGGAAATAATTATTGGCCTACACCTATTTACCACAAAACTGGACAGGAGTTTTGTACCC |
| | TAGGCTCCCTCCTCCCAGATGTATCCATCATTCCAGGAGATGAGCCAGTCCCTATCCCGACTTTCGAACA |
| | TATTGCAGGACGCACTAAACGTGCAGTCCATTTTATTCCCTTATTAGCGGGTCTAGACATCACCAGCACA |
| | CTTGCCACCGGGGTCCGCGGGGATAGGAACATTCCCTAGTACAATACCATAAATTATCTGGACAACTCAT |
| | ATCAGATGTCCAGGTACTCTCAGAAACTAATCCAAGATCTTCAAGATCAGGTTGATTCCCTAGCAGAAGT |
| | TGTCCTCCAAAACAGGAGGGGGATTAGATTTACTTACTGCAAAAAAAGGGGGCATCTGTCTGGCCCTCGG |
| | AGAAAAATGCTGTTTTTTATGCTAACAAATCTGGAATTGTTCGTGAACAGAGTCAAAAAAATTACAAAAA |
| | GACTTGAAAAAAAGAAGGGACCTCCTTTCCAACCCTCTCTGGACCGGATTCAATGGACTTTTACCCTACT |
| | TACTACCCCCTGCTTGGCCCCATACTCGGGTGCTTTATCCTACTATCACTGGGACCACATCCCTCCTCAA |
| | TAAACTCATGCGCTTTCTCAGACAACAAATAGAGGCCTTGCAGGCCAAGCCCATACAGGTCCATTACACC |
| | CGACGGGAGATGCAAGAGCGAGGAGATCCCTATCTCCCAATAACAGGAGTCATAAAACAGGACTCCTCCC |
| | CTGTGAGATGAACTGGATAGCCAATGACGGGTAAGAGGACAGCTCTCTAAGTAACATTAAAAAATCAAAA |
| | ACCTGTCGCTGTACCAGGTTTCACAGAGATGGACTGTCCCAACCTAAGACAGGCACAGTTCCCTAGGTGG |
| | CTCAGAGCTCTTTTTTATAAAACAGAAACGGGGGGACCTGTAGTGGGCGGGTGCCTGTAAGGCACCAATC |
| | ACATGACTGAGAAGCATGAGATAGAGGAAGTTACTTGGGTCTTTAGATAACACCCACATTCTGTAAGGTA |
| | TGTCCAGAGGGCTTAAGACCATCAGCCTGCGGCAACCCTGCTTATGTTAATGCCCCTCCACCCAGCACAA |
| | AAATGTATAATAACCCATGATTGAGCTGCAATAAAGAGAGACTTGATC |
| 2 | RFe-V-MD2 |
| | GGAGACCTCGTCGCGCAGCGGAGCGGTGCACCAGCCGGTCCTTCGTTACTAAAGGACTCAGGTGGAGGTA |
| | GGTGTGCGTTGGGCCGCTGATACTCGAGCTTGTGTGACCGGACTGCTTTTAAGAAATAGACATTTACACA |
| | CATATATAATTTAAAAAAGCAAACAAACATTTCAGGATGCATTACGTACCTTTATTGCCTGTCCTGCACT |
| | CTATTCAGTGTTCTGTTCCTTTGTCAGTTTTAAAATGTTGGTCCTGACTCACTGTATTGCTTTCATGACT |
| | CTCAGATGGGTCGCAACACACATTTTAAAAAATGCTGTAAGAATCCGGGAAGTGGGTGGTACCACGTTTT |
| | GACCGACTAGTGCCCCGTGTATACCTGCGTCAAACAGCACGTAGGTGTGAATGAGCCCAAGACCGGTCTC |
| | ACTGTGTCGTTGGCAGAAAAGAATCCTTGGCAGTTTCTGACAAAACTAAACAAAAAAGGATGAAATTCAC |
| | AGAAAATTTAAGTTATAGCCCTGCCTTAGTTATGTATCTTTTTGCACAATGACTAGGACTTTGGTAATAA |
| | CCTGTTTGTTTTCAACTTGAAAAATGCATAATGAATATCGTAGTATGTCATCAATAAATATTCATGTATA |
| | ACATACCTTTCAGTGACAGCAAAAGTTTGCATCCTACTGATGGACATTTTTAAAAGAAAAATATTTACTG |
| | AAGTTTAACAATTACACAAAAAGCATATGAAAGTGAACAACTCAATATATTTACACAAAGCAAGCAGACC |
| | CACGTACCTAGCACCCACTGTGAGAACCAAAATCATTACCAGAATCCCAGAGACTCCTGCCAAAGGTAGT |
| | GGGACCTCCCAGTCACTACCTTCCAAGCGTAATAATTATCCTGATTTCTACCACGGTATTAGTTTCACCT |
| | ATCCCTTCAGACCAGGCTGTCTCCCATAAACCACTGAATTTCTTTTGTCGCAACCACTTTTCTCTCCCTC |
| | TCCTCTCTCCCTTCTTATCCCTCTTCCTCTTTTCTCTGTTTAGGAGACCTGATTTCTCCATTTGCAAAAA |
| | GTATTTTTGCCCAACCTTCGTTTCACCTGGAGGTCTGTCTTCCTTTGCAAAGTTACTTTCTTGCTTTGTA |
| | CAACAGGCAACTGTCATCTCTGTATCCTTCCTTATCTGGAACTAGAAGAGAGTTAGAGTCGTGTAGTCGT |
| | GGCCGAGTGGTTAAGGCGATGGACTAGAAATCCATTGGGGTCTCCCCGCGCAGGTTCGAATCCTGCCGAC |
| | TACGGGGTTCTTTTTCTTCCCGAACCGCGAGTGACTCGGCAAAACCCGTGGCTGAACTTGCCGGGCCAGA |
| | GCTCCAGCGACGGGGAGGGAAGGTTCCGCGAGGAGCATGGCCCAGTTTCTGTCGCTCCTTCTTTTTAGGA |
| | CAGCTCTTCGTGAATTTTCCTCCCTATGATAAAGGGCTGCGGTCCCTGGGTCGCAGTCTCGGGTCAGCGA |
| | GAGATTCCAAGGGATCAGTGGGCCCAGCAGCCATCCTCGTTAGTATAGTGGTGAGTATCCCCGCCTGTCA |
| | CGCGGGAGACCGGGGTTCGATTCCCCGACGGGGAGGCAGTATGTTTTGTTTTGCACTCAGTCACCTGTTT |
| | TGGAGTTCCTGGAGACTCTGTGGTCCCTGCTAAGGACATGAATGCTACAGAGCTCTGTGTGGGTGCCACA |
| | GGTTCTGTGGGTCCTTCCCCTTGCAGCTCTCGGCGACCGCCCCTGCAGGGCTCTGGGGACTGAATGGCAG |
| | GGGACCTTCCTGTCAGCTCTTTTCAACTTGACCCTGCCCCCTGCCAGGCTTGTGCCACTCCCCGTTCTGC |
| | CGCTCTCTGATCAGAGAAACACTTCAGAGCGACTCTAAACTACCAAAACCTAGAGGGGAACTTAGGTTTT |
| | AAGTGACGCAGGACTTAGAACACTTACTGAGACTTAGTAAGAGTGTGGTTGTCTGCACGCGCCTCCCATT |
| | TGCAGAAAGAGCCACTGGGGGCAATGTGCGAGATGGCAAAAAAAATCCACGTGGGTCTTCAGGCCCTCCT |
| | TCCTCCTAGAGGTCACCTGGGAATGGGGACCGCCCACAGGCTCAGCTGGGGCTCTTTACTCCATCCTGGG |
| | CAACTGCTGCCCCTAGGCTCTTGCACCCAAGTGTGTGTAGGAAGGTGGTTAAGTGGTCTCGGACCTGTGG |
| | GAACAGGAGGCCTCCAAGTTCCAGGATACTGCTTTCAACAAGATCTGAAGCTCCTAGCAGTGTGCTTTTG |
| | AGTGTATGTTAGACTTTATGAACTAAAGCTTTCTGAAAGGAAAAAAAAAACCACTGTTATAAAGCCATGG |
| | CAGTCGAGACAGTGTGGCCCTTACTCAGGAATGGATAACTAAACGGATGGAACAGAACGCATCCTAAACA |
| | GATCCACTCATACAGCCATTTGGTTTAAAACAAAGGTGATGCCGCAATGCACTAGGGAAAGACCGTTCTT |
| | TTCAATAAATTGAAAATCAATAAATTGGTGGTTCAATTGGATATCAATATGGAAATAAATGAATTACAAC |
| | ATACCCCAAACTCAGTCACACGGAAGTATATTTAAACATCAAAGGGAAAGCAATAATGTTTCTGAAAGGT |
| | AACAGGATAATTTCTTCATGACTTTGGAGTATGCAAGAATTTCTAAAACAGCACAAAAAGCAGTCGTCAC |
| | AAAAGATAAGATATATGTATACATTACACTTCACCAATATTGGAAACTTTTGTTCATGACTAGCCACCAG |
| | TAAGCAAGTACAAGGCAAATGTTAGAGCAGGTGTTTGTATTACATGTACCTAATAAGAGACTGTGTCCCT |
| | AGACAGAGTTCTCCAGAGAAACAGAACCAATAAGAGGTATGCGTATGTAACAAGAGATCTGTTTTGAGGA |
| | ATTGGCTCACGCCATTCATTTCAACAATGTTTTGTGGCTTTCAGAGTATAACTTTTATACTTATTTTGTT |
| | AAATTTATTCCTATTTTATTTTTGCTATGATTTTTAAATGGAAGTATTTACTTTTGTCCTTTTTCTTTTC |
| | CTGTGAAACATTAGGAGGCTGACACCTCCCAGATGCAAGTATGAAGTGCTGAAAGATAGCAGGGATTAAT |
| | GTCCGCTAGGAGGGATACTCCATAAACATGCAAAGAAATATAGCCCACACAGGGAGAGTTTGAAAAAACT |
| | GCTTCAGACTCATAGGATAATGGCACAGATAAAGTGAGAAGCATACATACAATTGAAATGTGCAGTGTTT |
| | AGCTGGCTAGGACTTGAAGATGCTGATTGGAAGAAAGTGCTGATCCATGTCTTTCCATGTACAAGATGCA |
| | GCTCATGGAACTCGACCCTTAAAGTGGTGCCTGTTTGTTCTCAGAAGCAACAAGATAGAG |
| 3 | RFe-V-MD3 |
| | GAGAATTGGAGATGGCGGCGGCGCAGGGAACTTCGCAGGAACCGGCGGTTTCAGAACAGCCCGCTGAGCT |
| | GACTGCCTCCGTGCGGGCGAGCATCGAGCGGAAGCGGCAGCGGGCACTGATGCTGCGCCAGGCCCGGCTG |
| | GCGGCCCGGCCCTACCCGACGACGGAGGTTGCGGCTACCGGAGGTTCGGGCCCTGGCGGCGCCTGCCCCT |
| | GCCTTCTCCCGGCGGGCCGGGCGGTGCCGCGTCCCGTGTGTGGCGTCTACGCCTCCGGACTCCCAGCCCC |
| | GGGCTTTCCTCACTGCACCTGGGCGGTCCAGCTGCGGTCTTTAGCTTGGGGGTGCAGCCCCCCTCTCCGT |
| | CTGGAGGTGCCCACTAGTGCCCGTCCGCGCCGCAGCTCTCCCTTTCTGTTCTCTTCCGATAGCCTCCACC |
| | ATTCCCAGAGATGATGCTTGCAGAAAACTTTTAGACCTGTAACCCATCTCAGTAATCTGCACCCGCCTCT |
| | TCTTTCGTCCTCAGAGGGCACATTCCGGATCCAGCACAATGCTTGCCACGCGCAAGGCACCAAGAGGAGC |
| | AGAGAGACAGTAGCCACCGCCTTCGCGGGGCTCACAGAGTAGCCTCTGTTGTGCTTCATATGTTTGATTC |
| | TCGGAGCTAACCTGGAAAATTAGGGCAGGGTTTGGTATCCGTGTTGGTGAGGTGGTCGTTGCGGACAAGA |
| | AAAACGGGGTTTGCTTAGGTCCGTCTCAGTAAGTGCACAGGCTAATCAGGACTCGAACTCGGGTCATCCG |
| | ACACTGGGTTCAGGGCCTTTCCTTGCCACCAGCTGCCCCTGCTACACAAAGCACCTCTCCTACCCTTAGG |
| | AAGAAAGGCTGTTATTGTCTGGATTTCATCTTCCTCCTTTCTTAGGGTAGCTCTTCGCTGCGTATCTGTC |
| | GTGTATGTATTAATATGTGTAATTCTCCACTGTGGTCAAATAATAATCTTCCCCAGGGTGCCTAAAATAT |
| | AGTTTGGGTCTTCAGGGCTAGCTCTATAACGTGAAGTACATGTGTTCCTAAAGCTAATCCCATACTGTGT |
| | GAGTAGTTGAGCACAGTTTAAAGCTGTGTTATCTACTATCCTTTTGCAACAGTCAGAGTAAGGAAGAGTG |
| | ACCAGTCTGGGTCTGACTGCGTGTCTTGATATTGATACACTGAATCTGCAAATTCCAGCCACCTTTAATA |
| | ATTCTGGTCTTGTCCTTATTGCTTGTGTGTGTGTATGTTTTAATTCCTTTTTCAGCTTGAGGCATTCTAG |
| | AGTCAGGAGAAAAAGTTGTTCATTTGCATTGATTAATATTTATGATTCTATAAAGGATTCTAGATCTGTA |
| | CAGACAGTCCCCAACTTACAGTGATTTGACTTACGGTGGTGTGAATGTTATTCAGTAGAAACCATACTTT |
| | GAATTTTGATCTTTTCCTGGGATAGCCATATGTAGTACTATACTCTTGGGATGCTGAGCCACAGCTCCCT |
| | GCTAGCCACGTGATCATGTGGGTAAACAACCGATACTCTACAGTATAGTATTAAATGCATTTTCTTTTTT |
| | TAATGTTGTAAACATTAAAATATTATAGAGCAGAGATGTGTATTCAAAAAACACAGTCATAAACAGAAAC |
| | AAAATGTATTGGATGAAAAAAAGACAGTGCGCATTTGGGAAGGGTGATAGTGGAAAACTATTTAACACAT |
| | CATTAAATGCATTTTTGACTTAAAAAATTTTCTATTTATGATAGGTTTCTCTGGATGTAACCCCATTATA |
| | AACTGAGGAGCATTTGTACTAAATGTAGAATGGATGCAAAATAGAGTATAAACTAGTATTAAACTTCTGG |
| | TCATGGAAAGCAAGGTAGAATGAATATTCTGTAAGATTTCTTAGGCAGTTACCCAAGAAGTGAACTGTGT |
| | TGTAGTATTGCATACAACCCGCTGTGCTTTTAAGACTTAGGTAGGTACTGAGATTTTTATCTTCGCAGTA |
| | GTTTTATTTCAATGTACTGTACAATTTTCCATTTTCTGTATGTGCTCTGACATACACCATGAAAAAGATG |
| | GGGAAGAACTTGCTTAGAATGTGGTGCTAAGAAGTGGTGCTGAGGGCCTGGTGAAACAGCAAGGCATAGC |
| | AGCTGAGAAAAACTGGCATGATTTAGCATTGTTCAGGATCTTGCTCTAGTTTCAGCCTTGACTACTTTAG |
| | CTTCCCCTCTTCTTAATTCTCATTGCACTCTTGGTCATTCCAGTTATGTGCTACACGATTCATGAAATCA |
| | ATATCATTCTGGTATATTTATTGATTTCTATCCATCCAGTAGATATTCATGGAATGTTTAACTATCAGAA |
| | TTACAGAGATAAAACACTCAGTCTAATGGATGGATATACAGCCACCACTTCCGGAACCTTAGAAGTTTCC |
| | CTAAAGCCACGTTTTAGTCAATCAGCAACCCTCAGACATAACTACTGTTCTAACCATTTGATTAGTAATA |
| | GTATCTTTTTTTGAACCTCATGTAAATGGAATCATACAGTGCCTGGATAGTTTTGCTCAGCATAATATCT |
| | GCCAGATTCATCCATGTTGTTGCATGTTTTGGTAGTTTATTTATATGCTATATAGTTATTTTTTTTGTAT |
| | TATACCACAATTCTTCCATTTTTCCTTTTGGTGGATGTTTGGGTTGTTTGCAGTTTGGAGCTATCATGAA |
| | GAAAACTTTTGTGAACATTCTTTTAAAATTTTCAATTACATTTGACACACAGTATTAGTTTCAGGTGTAT |
| | ATCATAGTGATTAGACATTTATACAACTTACAAGTGATCACTCTGATTAAGTCTTGTAGCCATCTGACAC |
| | CATACATAGTTATTATAATATTATTGACTATATTTCTTTTCCCATGACTGTTTATAATTGGCAATTTGTA |
| | CTTCTTAATCTCTTCACCATTTTCATCCATTCCCCCACCCCCCTCCCATCTGGCAGCCATTCAGTTTGTT |
| | CTCTATATCTATGAGTTTGTTTTGTTTGTTCGTTTATCTTGTTTTTTAGATTCCACATTTAAGTGAAATC |
| | ACATGGTATTTGTCTTTCTCTGTTTGACATTTCACTTAGTATAATATCCACTAGGTTCATCCATGTCACA |
| | AATGACAAGATTTTGTTTTTTATAGCTGAGTAATATTCCATTGTATACATATACCACATCTTCTTTGTGT |
| | ATTCGTCTGTCAGTGAACTTTGGTTACTTCCATATCTTGGCTGTTGTAAATAATGCTGCAGTGAACATAG |
| | GGGTGTGTATATCTTTTCGAATTAGTATTTTGGATTTTTTTCAGATAAATACCCAGAAGTGGAATTGCTG |
| | GGTCATATGGTAATTCTATTTTTAATTTTTTGAGGGACCTCCATACTGTTTTCCGTAGTGGCTGCACCAA |
| | TTTACAAGGTGCTTTTCTCTACATCCTTGCCAACACTTGTTGTTTATTGATTTATTGATGATGGCCATTC |
| | TGACACGTGTGACATGATAGCTCATTGTGGTTTTAATTTGCATGTCCCTGATGATTAGTGACATTGAGTA |
| | TTTTTTCATATGTCTATTGGCCATCTCTGTGTCCTCTGGAGAAATGTCTGTTCAGGTCCTCTGCCCATTT |
| | TTTAAATCAGATTGTTTCGTTTTGTGTGTTAAGTTGTATGAGTTCCTTATATATTTTGGATATTAAACCC |
| | TTATTGGCATCTTCTCCCATTCAGCAGGTTATCATTTTGTTTTGCTAATGGCATCCTTCACTGTGCAAAA |
| | ACTGTTTAGTTTGATGTAGTCCCATTTGTTTATTTTTTTTCTTTTGTTTCCCCTGCCAGAGGAAACATAT |
| | TCAAAGAAATACTACTAAAAGAGATGTAAAAGCGTTTACTGCCTATATTTTCTTCTAGGAGTTTTACGGT |
| | TTTGGGTCTTAAATTTAACTCCTTAATCCATTTTTAGTTTATTCTTATATGTATACAGTGATCCAGTTTC |
| | ATTCTTTTGCATGTATCTGTCTATAGTTTTTCCAACACCATTTACTGAAGAGACTGTCTTTACCCAATTA |
| | TATATTTTTGCCTCCTGTCATAGATTAATTGACCATGTGGGCATGGGTTTATTTCTGGGTTCTGTTCCAT |
| | TGATTTATGTGTCTGTTTTTATGTCAGTACCATGATGTTTTGATTACTATGGTCTAGTAGTATAGTTTGA |
| | TATCAAGTAGCATGATACCTCCAGCTTTGTTCTTCTTTATCAAGATCGCTTTAGCTATCTGGGGTCTGTT |
| | GTGGGGTCTACAAATTTTAGGGTTACTTGTTCTGGTTCTGTGAAAATGCCATTGGTATTTTGATAGGAAT |
| | TGCATTGAATCTGTAGATTGATTTGGGTAGTATGAACATTTTAATGATGTTAATTCTTTCTATTCACAAA |
| | CATAGTATATGCTTCCATTTATTAGTATCTTAACTTTCATTCTTCAGTGTCTTACAGTTTTCCAAGCACA |
| | GGTCTTTTACTTCCTTAAATTCATTCCTAGGTATTTTATTCTATTTAATGCAATTTTAAATGGGATTGTT |
| | TTCTTAATCTCTCTTTCTGATAGTTTGTTATTGGTGTATAAAAATGCAACCAATTTCTGAATATTAATTT |
| | TGTGTCCTGATACTTTACTGAATTCATTTATTAGTTCTAATTGTTTTTTTGGTGGAATCTTAAGGTTCTC |
| | TCTATATAGTATCATGTCATCTGTGAATAATGACAATTTTACTTCTTCCTTTTCAATTTGGATGGCTTTT |
| | ATTTCTAGTCTGACTGCTGTGGCTGGGACTTCTAGTACTATGTTGAATAAAAGTGAAAGTGGCTTGTTCC |
| | TGATCTTAAAGGAAAAGCTTTCAGCTCTTCACTACTGAGTATGATGTTAGCTGTGGGTTTGTCCTATATG |
| | GCCTTTATTATGTTGAGGTATTTTCCCTCTATTCCCAATTTGCTGAGAGTTTTTATCATAAATAGATGTT |
| | GGATTTTGTCAAATGCTTTTTCTGCATCTATTGATATGATCATATGATTTTTATCTTTCATTTTGTTTAT |
| | ATAGTTTATCACATTAATTGATTTGCAAATATTGAACCAACCTTGCATGCCAGGAATAAATCCCACTTAA |
| | TCATGGTGTATGAACTTTTTAATGTACTGCTGAATTTGGTTTGCTAATACTTTGTTGAGGATTTTTGCAT |
| | CTATGTTGTTCATCAGGGATGTTGGGCATTTTTTTTTTTTTTTTGTATTGTCTCTGGTTTTGGTATCAGG |
| | CTAATGCTGGCCTTGTAAATGAGTTTGAGAGCCTTCCCTCCTTTTCAGTTTTTTGGAATGTTTGGTAAAA |
| | TTTACCTGTGAAGTCATTTGGTTCAGGGCTTTTGTTTGTTGGGAGTTTTTTGATTACTGATTCGATTTTG |
| | TTAGCAGTTACTGGTCTGTTCAGATTTTCTGTTACTGATTCAGCCTTAATTTTCTGCTGATTCAAGCCTT |
| | GGAAGATTGTATGTGTCTAGCGATTTATCCATCTCTTCCAGTTTGTCCAATTTGTCAGCATATAGTTGTT |
| | CTAGTGTTTCCTTATACTTCTTTGTATACCTGTGGTGTCAGTTGTCGTATCTCTTTCATTTCTGATTTTA |
| | TTTTGGCCCTCTCTCTTTTCTTCTTGAGTCTGGCTAAAGGTTTATCAATTTTGTTTATCTTTTCAGAGAA |
| | CCATCTCTTGCTTTTGTTCATCTTTTCTATTGTCTTTTTAGACTCTATTTTTGTTTCTACTGATCTTTAT |
| | TATCTCCTTCCTTTTACACACTTTGGGCTTTCTTCTTTTTCTAGTTCCTTCAGGTATAAGGTTAGATTGT |
| | TTATTTGATATTTTTTTTTTGTTTCTTGAGGTAGGCCTGTATTGCTATAAATTTCCTCTTAGAACTGCTT |
| | TCGCTGTGTCCCATAGATTTTGGGCTGTCGTGTTTTTATTTGTCTCAAGGTATTTTTTGATTTTCTCCTT |
| | AATTGCATTGTTGACCCAGTCATTGTTTAGTAACATGTTATTTAGCCGCCATGTGTTTGTGTGTGTTTCA |
| | GTTTTTTTCTTGTAATTGATTTCTAGTTTCATACCAGAGAAGATGCTTGGTATAATTTCAATTTACTGAG |
| | ACTTATTTTGTGGCCTAACGTGGTCTATCCTAGACAGTGTTCCATGTGCACTTGAATATACTGCCGCTTT |
| | TTGGTGAAATATCCTAAAATTATCATTCAAGTCCATCTGGTCTTATGTGTCATTTAATGTCACTCTTTCC |
| | TTGTTGGTGGGAATATAGTGATATTTTATTATGGTTTTAATTTGTATTTTCCCAGTGACCAGTGATGATA |
| | ACTTTTTCATGTGTTTACTAGCTATTTGGATACCCTCATTTGTGAAGTCCCTATTCAGGTCTTTTGCCTT |
| | TTTTTTTTTTTTTCAGTTGGGTAATTTGTCTTTTATTTATTTATAGGATTTCATTACATATTCTGGATGT |
| | GAATCCTTTGTCAGATATGCATCTTGCAAATAGCTTCTCCCAGTTTGCATCCTGTCTTTTCACTCTCCTA |
| | ATGGTGTCTTTTGATGAATAGAGGTTCTTTTAATCAAGACCAGTTTAACAATATTTTTTCCCCAATGGTT |
| | AGTACGTTAGGCCACTAAGAAAGTTTTAGCTATCTCAAGTTCATGAAGTTATTCTCTTGTGTTTTTTATT |
| | TTCTGGAAGCATTGTGTTTCACATTCAAGATTATGATCCATTAAAAAATGTTTTTTGGTGTATATTGCAT |
| | GAAGTAGGGTTAAAGTTCCTTTATTGAAAAGACCATTTTTTCCTCACTGTTTTGTAGTGTCACTTTTGTC |
| | ATAAATCCCAGTGTCATTTACTGAAAAGATTATTATTATTATTATTTTTTTAACCACAGAATTGCCTTGG |
| | AGCATTTGCTGTAAATTAAATGACCAAATATGTGTAGGTCTATTTCAAGATTCTCTCCTATTCCATTGAT |
| | CTCTTTGTTTGTCTTTGTGTCAGTATCACACTGTCTTAATTTATAGTAAATAGCTTTATAGTAAATCTTT |
| | AAAACCTCCAATTATTACATATAAATGTGAGAATCAGCTTGTCAGCGCCCACCTCAAGGTCCCCCCCCCC |
| | CCGATCCCTCCAACTACTGAGGTTTTGACTGGGATCATATTGGAGAGATAAATTTGGGGAGGCTGAGATC |
| | TTTACAGTATTGAGGCTTCCAATCTGCACATGGTATATTTCTCCATTTATTTAGGTCTTTGATTTCTCTT |
| | ACTGGTGTTTTCAGTGTAGACGTTTTATACATCTCTTCCTAGGTGTTATTTCTTAATTCTAATTGTAGAT |
| | TCCAATGGATATTCTACATACATAATCATATATTTGTGAATAAAGACTGATCTATTGCCAGCCTTGATGC |
| | TTGTTTTGATTTCTTACCATCGTGCACTAGCTGGCACCTTCAGATAATGTTGAATGGAAATGTAATAGTG |
| | GACAGTGCTTGTCCTGTTTGATATATATTAAATTTAGTGAAAGTTCCTGTTTCTACACGAGGGATCATAT |
| | GGGTTTACCTCGTTCAATTATTGACCACTTTTACTTATTTTTTGTAGGCATGGCTAATGTAAAAGCAGCC |
| | CCAAAGACAATTGACACAGGAGGAGGCTTCTTTCTGGAAGAGGAAGAAGAAGAAGAACATACAATTGGAA |
| | AAGTTGTTCATCAACCAGGACCTGTTATGGAATTTGATTATGCGATATGTGAAGAATGTGGTAGAGACTT |
| | CATGGATTCTTATCTTATGAACCACTTTGATTTGGCGACTTGTGATAACTGCAGAGATGCTGATGATAAA |
| | CACAAGCTTATAACTAAAACAGAAGCAAAACAAGAATACCTTCTGAATGACTGTGATTTAGAAAAAAGAG |
| | AACCAGCTCTTAAGTTTATTGTGAAGAAGAATCCTCATCATTCACAATGGGGTGATATGAAACTCTACTT |
| | AAAATTACAGATTGTGAAGCGGGCTCTTGAAGTTTGGGGTAGTCAGGAAGCATTAGAAGAAGCTAAGGAA |
| | GTTCGACAGAAAAACCGAGAAAAAATGAAACAGAAGAAGTTTGAT |
| 4 | RFe-V-MD4 |
| | AAGCAAATCCTAGAGCTTTTTGTTTTTTATACTATTCTATTGAAACAAAGTGGAAGGTTTAAAGAGGCAG |
| | CACATATACAAGTAGGTCAGTATCCCAGTCAATAAAAGTATTGTTTTATTGTCAACAAGCTGAATCTAAT |
| | GCACCACACACACATATATACACATCATCAGATAGATACAGACTTGGTTAATTTGATGAGTGGAGCAAAT |
| | GAGAACTAGACTGCTGCATCTACTGTTTTCTATGGAAGTGGACATTGAGCAACATAAATAGCTGATCAAA |
| | GATCTATAAGCACTGTCAGGAAACAAGAATTCCAGGTGTTTTCATGCTGTGACAATGAGCAACTCCAAGA |
| | AGATTAATCAGAAAAATGCATACCAAAAAAAAAAAAAAAAAAAGGAAGAAAAAAAAAAGAAAAATGCATT |
| | CCTACTCACAACCATACCATTTTGTCTTTTGTGAACTCCGTGTGCTGTCTTGGCGGTAGTGTGACACTGG |
| | AGAAATCTGTCCAGCAGCATCCTCCCTGTTAGATACCCTCACTCTTTCAACCTACAATGAAATATATTGT |
| | TTCCACTGAAATATCACGAGGGCCATCTACACAGCTTTTTCACGTTTTTGGCAGACCTCACTCCTTAGTG |
| | AACTCCTGGGGCAGTAACCTCTTCCTTCTCAAAATCATCTGGATGAATCCTCCTGTTATTTGAAAATCAT |
| | CTCACTGAGCTTCAAGGGTCCTCTTGTGAATTGTGACCATAGCCTACCTCATATCAACAAAAGTTTCCAA |
| | TATGAGGTGTGGAAAGAGGATAAACTTTATTCAGCTGAACAGTTGGTAAACAGGAAAAACCGAAAGTGCA |
| | CACCAAGACAAAGGGGAAGGGGCCTTTTACAGAGAAAGTTAGTGCCCTGGTTCCCATTTGGTCCATTTTT |
| | ATGCAAATGAGAAATCCAAATCACACAGTTCTGATCAGTCAGCATCATATGTTCTGATTGGTTGTTGTGA |
| | ATCAGTTCTGATTGGTCAGTATAGATGCAAATGAGGATATAACGCCACAGTTCTGATTGGATGGGACTAG |
| | TCTCAGTCCTTTGGAAGTTCCATCAGGAGTTCCATCAGGAAGTTCCTGACAATGGTTGACTTAGGCAGCA |
| | GCAGGAGCACAGTTCGGGAGGTGGAAATTTCAGTCTGTGGCTTTTCCCTGAAATGCAGAGTGTGCGAGAG |
| | GCTTGTGTCAGGAATGGCTGTTAGACTCTATTAAGAATTTGAGCTCAGTTAACCATGAGGAATCCTTCTT |
| | GGCAGATTATTTCTTCTCAAGGTTCACACTTATGAGGGAGACTGCTTCAGAGCTTCCAATGAAAGGGCGG |
| | GTACAAGGGTGGTGATTGGACTACTGATATGTCTTTCAGCCATAAGGCTCACATTGATGCTGGTAGGGAT |
| | CCCATTGCATCTGCAGATGGATGTGTGCTTTACGATTTGAGAATTGACTCTGACCCATGAGAAAACAGAG |
| | CTCGAAGACTGGCTGAGGAGGGTACATTTGGGTCAATGTGACACAGAGTATTAAAGTTAAGGCACACTGT |
| | TGTCAATTCATGTATTCAGAGTTGCTCTGTAATGTCCACAGTTTTTTAGTTGTTCTTCCTAGAACTTCTT |
| | TCTCAGGAAGCACTTGAAACTTCATTGTAACAGATGAAACCAAGAAGTCATTTTAAGCTCTTTTTTTTTT |
| | TTTAAACTCTTTTTAAAAAGGTATTTTAGTGTTTTGTTTCTTAGTTGACTAAGAACAATGGCACATCATT |
| | ATATTAAATACTAAAATTCAGTGGTCAAATTGGCTTATTTGAAATTTAGAAGGTAAAGTGAACTTTGGCC |
| | AAATTCCTTTCAAATGTAAAATAATTTCATTGTGATTCACTCAGCAACACTTTGAGATTAATTTGGGATT |
| | TGGGGATCAAAAACTATCAAGCTTTTAGGTTGATGGTTAGAGGACTCTAGAACTATAATTATTAATTTCC |
| | TTGGTTGTGCCAGACAGAGTTGGGCATTATTGCTCAGAAATGAATAAATCAAAGTTGTTTTGCATGAGAA |
| | ACTCACAAAGTTGCATGAGGGACAGAGTGGGTGTTGAGTGCTAGAGTGAAGGATACAGAGTGTTAAGCAA |
| | GTAAAGAGAAGCAACCCAGAATAAACATAATGCCAGAACACATTTCTAAAATTAGGTTATGCTAAAGATG |
| | ATTCTAAAGAAATATGTGGGTGTGGCAAGCAAAATAATGGCCCCTCAAAATGTGCTAATCCTAATCCCTG |
| | AAATATGTTAACATGTTACTTTATACAGCAAAATGGACCTTGTACAAATGATTAAATTAAGACTATTGAG |
| | ATGGGGAGATTGTTTTGTATTATCTGTGTGGATCCATTGTAATCTCAAGGGTCCTTGTAAGTGAAAGAGG |
| | TAGACAAGAGAATCATACAAAGAGATGTGATTATGGAAGCAGAGGTCAGAGTAATGTGGTCTCACATGAT |
| | GCCAAGTTTTGGAACTGGATGAGTGAGTGCCATTCAATAAAGGAGGGTCAGGTATTATTTGTTAATTCTT |
| | GACATCCATTTGCTTTATTCTGACAGCAGCTCTGTGTTTCATTTGAGGTTCTGTCCCTCTTTCCCCCACT |
| | CTCAGCCCGTGGGAGGTACCCATGAGCCCTGCGATGATGTGAAACGGCTAAACAGAGCAGTTCATTGCAT |
| | CTCTCTGGCTAACGTATTTGGTTCAGTGTTGGACATGTGACCTTAGCCGTTCTAATCTGAGTGACTGTCA |
| | AAACTTTGGTGGAAATACTAGGAAAATAGTAAAAACAGAAGCTGCACAGTTCTTTTCTGCCTGGTTAGAA |
| | TCTGGAAGCATGCAGTTTAGGGAGATGGTGGTAGTCATTTGTGGTCACAAATGACCAGCATTCTGAAGGT |
| | GAAATTAAAAAAAAAAAAGAGAAATGAGAAGGAACTAGCAAAACAGAAATGGCGCATGATCAGTGAGACT |
| | TGGAGCTTCTGCATCCAACGAGTCTTATCCTGGAACCAAAAGGTTATTTTGAGTTTTTTGTTTTTGTTTT |
| | TTCTACACAATTTGATTTTGACTTTCTCTTACTTGCAATCAAACTAATCTGAAGAGAGTACAGAAGAAAG |
| | GGCAGGCATGGATGTTTAAATTTAAAGACATCCACGTGGATTATGCTGTAAGGAAATGGAAAAATGGATT |
| | TAATGATCAGAAAGTAGTGTATATAGAAGATGTTTATTTGGGATTTATCAGCTCATAGATGGGAGAAAGC |
| | CGGGCATATTGATCATATTGAGTGAGACTAGAAGGGGTTTAAGGTCAGAAGTTGAAGAATACCAATGTTT |
| | AATAGTCAGGCACAGTACAAGAAAACTTCTAAAAGACAGGGAGAAATCATTGCCAGAGACTAAACCTAAA |
| | TTTGTCAGTTTTCAAAAGTGTAGTGTAGAGATTAAATAAAGAGAAGACACTTTAAGGAAATTTATTAAAA |
| | TGTGAAGCAGTGCTGTGTTTTTGTCTTTGGATATTGGGAATATGAATGATTTTTTCTCTTTTCACCTAAT |
| | TTTCTGTATCACTTCTGAAATAAACAATACGTTTTGTTGGGGTGGCCTAATGGCTCAGTTGGTTAGACTG |
| | TGAGCTCTCAACAACAAGGTTGCTGGTTCAATTCCCGCATGGGATGGTGGGCTGCGCCCCCTGCAACTAA |
| | AGATTGAAAAACGGCGACTGGACTTGGAGCTGAGCTGTGCCCTCCACACCTAGATTGAAGGGCAATGACT |
| | TGGAGCTGATGGGCCCTGGAGAAACACACTGTTCCCCTATATAGCACAATAAAAAAATTTAAATAAAATA |
| | CTCATAATAAGTCAACATAGAACATTGACTGTATTGAAAATCTTGAAATGTTTGTCAAAATATGGGGTCT |
| | TAAAATTAAGTTCGAGAACTTGCCACCTTGCGTTTACATTGGCAGCACTGTACAAACAGCTCGATAAGGT |
| | TTCATAACCTTGGTATATAAATCTCACAGCTGTGTCCGTGTGGACATGTGGCGGTGTTGCTGAATGGCAT |
| | TCATTATTGTTGTTGTGTGTTTTTGTGTTGCATCGCAAGAATGTCTGAGCTTGAATTAGAACAATGAACA |
| | AACATTAAATTTCTTGGTAAACCTGGCAAGAGTGGAAGTGAAATCAGGGACGTGTTAGTCCAAGTCTATG |
| | AGGATAATGCCAAGAATAAAATGGCAGTGTACTAGTGGAGTAAACGTTTTTTCCGAGGGGAGAGAACGTG |
| | CAACTGATAAAGAGAGGTCAGGGCATCCAATAACGAGTAGAACTGATGAAAAAAATTGCAAAAATTCATC |
| | AAATGATCCATCAAAGTTATTGGCTGACTCTGAGAAGCATAGTAGTCCAAGGTAAAATCAATAGAGAAAG |
| | ACAAAATCTGAACTGAAAATCTTGGCATGAGGAAGATGTGTGCAAAAATGGTCCCGAAGTAGCTCACCGG |
| | TGAACAAAAACAAAAGAGAGTCCAAGTTTGTCAAGACCTTTTGGAGAGGCAACATGACATTTTAGGCCAT |
| | GTTGTCACTGGTGATGAAACATGGGTGTACCAATATGATCCTATAACAGAATGTCAAAGTACAAAATGGA |
| | AGTCAGCCAATTCTCCACGAAGAAAAAAGTTCCATCAGTCCAAATCAAGGGTCCAAACGATGTTGCTGAC |
| | CTTTTTTGATATCAGAGGGATTATTCATTATGAATTTGTACCAACTGGACAAACAGTTAACCAAGTTTAC |
| | TATTTAGAAGTGCAGAAAAGGCTGCGTGAAAAACTTCAGACGAAAATGGCCTGAACGTTTCTCCAACAAT |
| | TCATGGATTTTGCATCATGACAATACACCGGCTCACACAGTCTGTGAGGGAGTTTTTAACCAGCAAACAA |
| | ATAACCGTATTGGAACACCCTCCCTACTCACTTCACCTGGCCCCCAATGCCTTCTCTCTTTACCTGATGA |
| | TAAAGGAAATATTGAAAGGAAAACATTTTGATGACATTCAGGACATCAAGGGTAACACGACGAGAGCTCT |
| | GATGACCATTCCAGGAAAAGAGTTCCAAAATTGCTTTGAAGGGTGGACTAGGCGCTGGCATCAGTGCACA |
| | GCTTCCCAAGGGGAGTACTTTGAAGGTGACCACAGTGATATTCACCAATGAGATATGCATTACTTTTTCT |
| | AGAATGAATTCACGAATGTAATTGTCAGACCTCGTATACTATAAGACAAGAATCGTAACCTCCAGTGCTT |
| | ATGGAGACAAAGAAGGTGACCAAAGTAAGTGAAGAACCCAGGTGGGGACAGTAGCAAACTAGAGAACACA |
| | TGTCTGATCTAAAAGGCACAGCACAGTAAGTGATCAAGAAGGACCAGGTTTGATTCTTTAGAGAAGCTTG |
| | ACATCCACATTCTACGTGAGTCTCCAAAATTGTCAGCGTTGATCAATACATGGAGGCAAATTAAACATAT |
| | CCAGGAGACACATTTAGTCTATAGGGCACTTGGGATTTTATATTTGCTGTTTCCAAATGGTTGTGTATAA |
| | TGTGAATATTTGTATGTAAAATCTTTCCTTTCTTTGGTATCCTACGTTTTATCCAAAAATTGGGCCGCAG |
| | CTTGCAATAAAGACAGCTTGTCATTTAGACTCATTTTACCCACTTCAGGAATTTTTCAAAACTTATTCAC |
| | ACCACAGTCCATTTGCATTTATTTTTCACAGATTGTTAATCAAATACTCAATTCCTGCATAGGACCGCTG |
| | ATTCTAAATTATTGAAACAGTTCCGTTCTGTTTTGGTACGAACTCCAGGTTCTTGATGTTTTGATGTTTA |
| | AACCTACCCTCCTGATTATGCCAGGGCTGTAGGAATTAAACAGACATATTGAGACAGTCTATCGCACAGC |
| | TTCAACTAAAAGGAAGGTTCATGATTTCTTACTGCTGCAGGAAAAGCATGCTGGTGGTAAACATTTATTG |
| | ATCTAACGACCTGAGCGTGAACAGAGATGCAAAACTCTTTCTTCAAGGGTCGGATTCTACTTATTAGTAG |
| | ACTACCCATCAGCAAATGTCTAAAGAGTCTCTGAGCGCCAGTGAATGACTGATGGCAAAAAGGAAACAGG |
| | TGTACTTCTGTAGGCCAGCAGATACCGCCAATGATATCCCTTTCACTTCTCGAGCCCACTGGTAAAGACA |
| | GTTCAAGTCAGCCTAAGCGTGTTGCAAAGGAGAGAGATGAAGTAAGTACCCCTCACTAACTGTACCTTTT |
| | CTAGAGGTTTCTTACGCTTTTGAAATCTGTGAAGTGATACATTACACTTATACATTCAGTACTTTTGAAA |
| | CAAGGGTTGTATCAGAAACTCGGGGAACTATTTCTAAATACACAATGTCCAGGCCTTATTAGATTGACTC |
| | AGTCAAAAACCTTCAGGGAGGAGTCCAGGCCTGTAAAGGTTTGTAAAGTTCCTCAAGTGACTGTGAGTCG |
| | CCAGCACAACTCTAGCTGAGAAATACTGCAGTAG |
| 5 | RFe-V-MD 5 |
| | TTCCCTCCTCCACTTACACCTGGAATGGTTGGATGGGTCCAGTGACATAGAAGGTGTGGTGGCTGGCAAA |
| | ATTCTGCCATACTTTGGGGTTACATGTATATAGATGTTAACTACTATACAGATGTGCCAGGCATTGTTCA |
| | CTATGTATTACATATTGAATTTTCACAATAATGTTAAGAGGCAGGTACAATTAATACCTCCACTTTCAGA |
| | TGAGAAAATTAAGGCAGAGAGGTTACATAATGTGCCCAAGGTACCACACCTTGATAAACAGCAGCTGGGA |
| | TTCTCACCCATCCAGTCAGCTTCAGAATCTGTGCACTTAACTACTAGATGCTATATAGAATTAATGCCAA |
| | AACTCTCAAAATCAGAGTCATGAGAGAAAAGCCAAAGCCATCATGCCAATATTTGTTAGGTTAGGTTAGG |
| | CTATGTTAGGTTCGTTTTATTTTTTATTCCCCTAATTTCCTAATCTTCTACATTTAGGGGAAGAGATGTG |
| | CTTCTATATTCATGAATGTTTATGAATGAACATCGTATGGGACCATTATAACTGGACCCTAAGGAGATAT |
| | GTTCTTGACATAATTCATTATCAATGATCAGCATTCTCTTTGGGTTGATTGGCCATGTCTTTATCATCTC |
| | CACGTCCTATAGAACTGTTCTTATGAAGAATATAGTCAGGACACACACACACATACACACACGCGCGCGC |
| | GCGATGGGGACTCTTAACTAGCTCACCCCCACCAAAAAGCCTCATCTAGAAGCACAGAGTTATCATGAAA |
| | TACTCTGGGGGAGGGGGCATCATGGGGGTGGCAATTCAAAGAGAAATGAGAAAAATCACAAGATGTTTAA |
| | ATCAATGGGGATAGCGCTGGAATTTTCCATCCTGAAGATTTTTTCCAGGGCTAAACCTCTGACTGAGTTT |
| | TGTTTCTTTAACAAAGGAGGTGGTGGTGGTGGTAGATTACCTTATTTTCAAAAACGTTCTTTGTAAACAT |
| | CCAAAATTATTTCCATGAAAATTGTTTCTCTTACATGTGACCTCAATTGTACTCAGCTGACCCTGTGACT |
| | ACTTGGAGTTGTGGTGGAACAAAGTGCAACAGTTTCCTCCTGGAAGTCTTTCATTTTCATTGTATGAGGT |
| | GTGATAAAAAAAATACAGTGAATGTTTAAATAAAAAATTTATTACAGTAAAAGACACATTACCATTAATT |
| | CTCCTCAAAATACTCCCCCTTGCTTTGAACACATGTATCCCTTTGTTTTTGCCACTTTCTGAAGCAGTTC |
| | TGGAAGTCCTCTTTTATGAGTGTCTTTAATTGTACTGTAGTGGCTGCTTTGATGTCCTGAATCAATTCAA |
| | AAAGTTTACCTTTTGTGGTCATTTTTTCTTTAGGGAAGAGCCAGCAGTCGCACGGTGCCAGATCTGGTTA |
| | ATAAGGCGGATGAGGACACACCATAATGTCTTTAGTTGACAGAAATTGCTGTATACCAGAAGCGATGTTG |
| | GAGCATTGTCATGATAGAGGATGATTTACAGCACACTGTAAAACACACCTTCTCTCAAGTGTATCTCACA |
| | CCCAACTGACTGCACCAAACAAGTTGAAACTTGTCACACATCATTACTAAGGTTTGACATGCAGCTTCTT |
| | GTATTGAATATCCCTGCCTTTCCATTGGATGGCACTTAGCAGCAACGTTCACTGTATTGTTTAATCACAC |
| | CTCGTACTTATTCTGATGGAGAAATTTTTGTCAGTTGAGCACACTTTCCTCTCTCATCCTTTTATTTTCT |
| | GTGTCTAGCTTTAGTTTGGGATGAGGGAGGACAAAGTACTATTATTATGAAATTACAGTGGCTCTGGAGG |
| | CCTCTCAAATCCTGACTATGACACAGAAAATTCTGAAATAATTCACAGCAGGAGTACTATAGGACTTGGT |
| | CAGCTTTGCATTGAACATAACTCCACATCTATATTGGCTCTGCTTTTGCTTCTCAATATTAAATGCTCAA |
| | ATATGTCAGTGCTAGGCACTATTATTTATATCCCTCTGAAACATGTTTCTATTCAAGGATGCAGCATTCA |
| | GAAGACTCAGTCCAGCGAGTGACAGAAAAAGACTTCCCTTGGATTATCTATGAGATTGTAATAGCTTATC |
| | TGCATATCTGCTCACTGAATACTGCCTCGATCATTCATATATCTGGCTCACAATGGGTAATCAATAAATG |
| | TGTGATGAATGGTCTACAATTCCCAGATTGCAGCCCTAACTTGCTCATGATGGCTTCCAGTAGTTTTCTA |
| | TCAAAGCCACATGTGGTCAGTGTGCAGGATGAGGAGTCGAGCCCTTAAAACTCAACTCTAGAAGACCTAC |
| | TGAAGCAGTTATTACAACATGCTACAATACACAAAGAACAAGACTTGTACATCAGAAACAGGTTGTCTGA |
| | AAAAGTTTTCTATTGGGGGAATGAAGCAAATTGAGCCTAAGTTTTCTGGACAAAAAGAAAAGGCTGATTT |
| | ACTCAGTTTAAGTCTAAGACCAAAGAATAAGTCTGAGAAAAACAAGATGTTACCTGATCTTATATGCACT |
| | CTATTATTATTTTTGCTTTGCATGTCCCTTGTAATAGTGATTGGTTTTAATGATCATTTCATATAAAAAT |
| | TAAAAAGAAGTACATTTTTTAACTTCCTGTCAAAAATTCTGCCTAAGGTACTTCCTCAACACACACACGT |
| | TAGTTGCTACCCCTCCTTCAAGGCTCTGTTCATGCCCGTCTCCTCCACGAAGACTTTTTTGTTCTACACC |
| | TAGAAAGGCTCTGCCTACTCAGGCAGTTGTTATTACCTCCGATTTCCTACTATCAGATCTCTTCGTATTA |
| | TCTTCTTATATGACTAGGTCTCATCTCCCCCTCAACCACAATCTCTCTGAGGGCTGGAATATTGTGCACA |
| | TTGCCTTGCACATAATAAAGGCTCCAGAGGTATCTGTCTAAACTGGCTTTATTTCCTTGAGACTACAAGC |
| | ACTTATTCTGTGCCAGGCACTTTTAGGTTCCAGGGAAAAAGAGGTACAAAACCAGACACAAACCCTACCG |
| | TTATGGAGCTTACCTTTTTAATTAAAAGGTGGAAGGGATGAACCTTTTTTTGGTCTCTCTAGAAAGTTGC |
| | AGCAGGAGACCATAGGAAATAGTATAAAATAGTTGAAAGCACTGTGGAGTGTGAGTCAGGATACCTTGGT |
| | CTCATCTCTAATTTGATGTATCTTGAGCACATTTCTTAAACATTGGTCATCTGTTTCCCTGTATGCCATA |
| | TAGGAATCATATGGTTACTGGGAAAACTGAATCAGAAAACAGATGCAAATCATGTTGGAGGGAACTTTCT |
| | CAACCTGATAAAAAGCATCTATGAAAAACCCACAGCTAACACCATACTTAAAGGTGAAAGACTGGAAGCC |
| | TTCTTCCGAAGATCAGTAACAAGACAAGGATGTCTGCTCTCACCACTGCTATTCAACATTCTACCGGAAG |
| | TTCTAGCCAGGTTCTAAGTAAGAAAATGAAATAAAAAGAATCAAGATTGGAAATGAAGAAGTACTAAAAC |
| | TATCTATTTTCATATGACATGACCTTACTTAGAAAATGCTAAAGAATCCACCCCCAACCCCCACCCCAAC |
| | AAAACTATTAAAGCTAATAAGTGAATTCAGCAAGATTTCAGGATACAAGGTCAATACGGAAAAAAAAAAG |
| | TTGTATTTCTATAAACTAACAATGAACAATCTGAAAATGAAATTAAAAAACAACACCATTTATGATAGCA |
| | TTAAAAAGAAATTAAGGAATAAATTTAGCAAAGAAGTGTAACACTTGTACGTGGAAAACAACAAAACATT |
| | GTTGAAAGAAATCAAAGACCTAAATAAAATTTTTAAAATCCTGCCTTTGTGGATTAGAACACTTAATTTT |
| | GTTAAAATAGCAGTACTCCTCAATTTGAATTATTCACAGCAAATCCTACAAAAATCTTAGCTACCTTTAT |
| | TTTCCTGCAGAAATTGACAAGCTGAGTTTAAATTTTACATGGAAATGCAAGGAACCCAGAATATCCAAAA |
| | CAATCTTGAAAAAAAGGAACAAAGTGGGAAGACTCATACTTCCTAATTTAAAAACTGACGGCAAAGCTAC |
| | AGTAATCAAGACTATGAGGTACTGGCATAAAGACAGACATATAAATCAATGGAATAGACTATGAGTCCAG |
| | AATAAATCCATGGTCAATTGATTTTTGATAAATGTGCCAAGACAATTCAATGGAAGAAAATAATCTTTTC |
| | AACAAATGGTGCTAAGACAACTGGATATCCACATGCAAAAGGATGAATTTTGAAACCCTACCTCACACCA |
| | TATACAAAAATTAGCTTGAAATGGATCAAAGATATACAAATAAGTGTTACAACTATAAAACTTGAAGAAA |
| | ACATAGGTGTAAATCTCCATGACCTTGGATTAAGCAATGTCTTCTTAGATACAACATCAAAGCACAAGCA |
| | ACAAAAGAAAACAATTGGATTTCATCAAAATTGAAAACTTTTGTGAGCCAACCCTCACAACCCTCACACG |
| | GTGGCTCAGGTGGTTGGAGCGCCATGCTGGTTCGATTCCCACGTGGGCCAGTGCGCTGCATCCTCTACAG |
| | CTAAGACTGTGAACAACGGCTCTCCCTGGAGCTGGGCTGCCACGGGCTGCCGTGGGCTACCATGTGCTGC |
| | CAGGAGCGGCTGGTGGCCAGCGTGAGTGACCGGCAGCCAGCGAGAACTGACATGAAGTGCTGTGAGTGGC |
| | CGAGAGGTCCAACCAGTAACCGACTGCCTCAGCTGGGGGGAGCGCAAGGCTCATAATACCAGCATGGGCC |
| | AGGGAGCTGTGTCCTACATAGCTAGACTGAGAAACAATAGCTTACGCCGGAGTGGTGGGGGAGGCGGAAG |
| | GGGAAAACAACAACAACAACAACAACAAAA |
| 6 | RfRV |
| | AAATTAAGACTCACGTTAGGGAAGGCTGAGACAAGCAGCAGAAACCACTAGATAGGAACAAGAAATGTGA |
| | GGAAATCAAGGCAGGGAGCATGTGAAGTGGCAGGGAGGGGACAATGGAAGAGTGAAACAGAGCAGAGGTG |
| | ACAGGCAGCAGAAGAGAAAGTGATTAGAAGAGAAGGTGGTACATTAAGCTGTTGGTAATAACAGAGACAA |
| | GAAATCGCAATAGAGGAAGAGTGTTGCTTCTGAAAGGAAAAAATCTAAATTAACTAACTAAAAGCAATCT |
| | ACGATCACAACTCTACCTGTTAGGAGCAAATAGCACTATATACCTACATACCTCTGTCATCCCACATGCA |
| | TTACAGTGCTGCCCTGGACAAACATGAGGGTGAATAAGTCCCCGCTTTCCCTGGGAATGTCCCAGTCTTA |
| | GCACGGAAAGTCCTGTATCCCAAGAAAACACACACACAGTAGCAGTCTAATCAGGACAGTTGTTCACCCT |
| | GATTAGCATTGACTCAAAATAGCAGTGCAGTTTGGGGCTGGTCTGTAAAGTGTCCCCTTAGTGGTACTCA |
| | GGATTATTACTGCTTCACAGTAACCACACACATGCTAGTAAGTGTTAAGATCCGGAATTGTCCCCCTCAG |
| | ACAGACAGAGAACCCGCACAATAATGCAAGTCACACAGCGAGGTTTATTACCAGCCGGCTGGGGTCCCCG |
| | TCTCTGCCCGACGCAGCGGGTTTTTAACAAGGACCCCAAACACTTAAAGCCAAGGGGTTATATAGTATTT |
| | TTAAGGCCTTCCATAGCTTCTGAGAGTACAAGATAAACTTACAGGACTTACATAGTTTCAAAGAGTATAA |
| | GATTTACTACAGGACAAGGAGACCAGGAGTATAAGATAAGCTACAGGACTTCTAGTCGCCATGCTGCAAC |
| | TGCCCACATCCTGGAATTTTATAATTATGTTGTTTCAGGCTAGGGGCTGTTAACCCTGACCTGAAGATAG |
| | CAGTTCTCATGCTAACAGTCTCTTACATGCTAACAGTCTCTTACATTTCCCCCCTGTGTGTTGTTCATAA |
| | TGAGGAATCTTGCTCATGTACGGGCCAGCCGTAGAGGTCCTCACGGGTGGGCACTGTCTTATACTGTTGT |
| | CTTAGGACCATGAGCTGGACAGTATTAACACGATCTTTTACAAAATTAATAAGCTTGTTTAATATGCAAG |
| | GGCCCAAGGTTAGAATAAGCACAAGCAAGATAATTGGGCCCACTAAGGTAGATACTAAGGTGGTGAGCCA |
| | AGGAGATCTGTTAAACCAAGCCTCAAACCAGTTCTCCTGAGCTTCCCTTTCCCGTTTTCTCTTAGCTAAC |
| | CCTTCTCTCACCTTTGCCATGGATTCTTTTACCACACCAGTATGGTCAGCATAAAAACAACATTCCTCCC |
| | CCAGCGCGGCACACAGTCCCCCTTGTTGGAGGAACAACAAATCAAGTCCTCTTTTATTCTGGAGTACTAC |
| | CTCGGATAGAGAGGTGAGCGACTTCTCTAAATGACTAATCGAGGTTTCCAACCTTTCTATGTCCTCATCT |
| | ATGGCTGCCCTTAGGGAGGTCATTCCTGATTGTTGAGTAGCCAGGGAAGCTATGCCGGTCCCAGCTCCGG |
| | CTATTCCCAAACTGAACAGGGTGGCAATGGTTAGTGCGGTGATGGGCTCTCTTTTACTTCTTGTGTCACT |
| | ATCCCAATGCGAATACATACTCTCCTCAGGGTGATAGAGGATGCGGGGCAGCACTGTTACTAGGACACAG |
| | AATTCATTGGCGGCATTAAAGACCGAGGTAGATAAGCACGGGGTGAGGCCAGTCTTTGAACATATCCACC |
| | ATCCATCAGTTCTGGGGATTAACCACTTAGTGTCACTTTTCCAACTAGGGGAGCTGTCTATGGAGGCACA |
| | TAAACTTTGTTTAGCCTGGGGCACCTTTCCTAGACAGGTCCCATTGCCGCTCACTAGTTGCATGGTTAAG |
| | CCAATTTTACGATTTCCCCATGAACACTGAGAAGGGTTCTTACCGTTAGAGGCGTTGTAAGTGGCATTAA |
| | GTCCTATTGCCTCATAGAACGGGGGCTTTATATCATAGCACAGCCAACAGGAGGTTGTGAGGTTAGGACT |
| | GGTGGCATTAAGGGTCTCGTATACAGTGCGCACCAGTTTCCGCAATGAGTCTTTGGTTGGCTGCGTAGCA |
| | GGCGTCAAAGGAGTTACAGAGGTCTTTGGCTGGGTTCCCGCAGTCCCTCCTGCGGTGTTTTTATCCCTTG |
| | ATATACCTGGGTTCTTGGTAGGGACGAGAGGGGCCAGCACCTTATTTGGACCCACCTGAGTGCTGATCAT |
| | TTCCACCGATAGTCGAATAGTTAGGAGACCGCCGGGGTGGGGACCTATCCATGCCCAACGGCCTATATCT |
| | AATTGGAACCCCCAAGTTAATCCTGATAACCAACCACGCTCTCTTCGCGCCACGTCTTGGTTAAACTGGA |
| | CACGTACCTGGGGCACCCGGTTGTGGGGGTCCCTAAAGGAGAATTTAACTAGATCCCTGTTCCCAACGTC |
| | CCACTGTCGGGGCCCGTCATAGGAGGTGACACAACTCCAACTACCACAATAGTAGCGGTCTGGGCCGCCA |
| | CAGGTTTTCCAATTGTTTCTTAGGTTCCCTGGGCAGGCCCAAAACCCTTGTGCACTATGTCCTTGAGAGG |
| | TGTCAATTACAGCCCGCTTGGACCTGACTGAGTAGTCATACTGCCGTCCACGTTTAGTGCCGAAAATGTC |
| | ACGCAGGTCGAAAAACAAGTCTGGCCACCACGTATTGATGGGGGCAGTATGTGTGGTGCTATTAAGGGTT |
| | GTTTGGGTCTGTCCATCTGTTAGGGTCCATGTTAGCTTATGGGGTTGGTGTGGGTTGATCCCCGCGTGGC |
| | TCTTCTCCCAGATATTGAGCAGAGTTAGGGCTAGCAGCCATTCCATCGTTAGCTGAGGCAGGGGGCTTGA |
| | CGCTTCCCCGAGGTCGGGAGAGCTGCAGCTTCAGAGGGTTATCAGGGTGTCGCCGTACGATCCACTCCTT |
| | AGCTTGCGTCTTCTCCAGCTGGCTGGCTCGGCGCACGTGAGAGTGATGGACCCAAGGCCCAATGCCGTCA |
| | ACCTTTAAGGCAGTGGGGGTAACCAGAATAACCACATAAGGACCTTTCCATCTCTCCTCCAGTGTCCGGG |
| | ACCTGTGTCTCCTTACCCATACCCAATCCCCTGGAACGATGCCATGTTCCGGGTTTGGGGCGTCCTTAAT |
| | TTCATACAGGGAACTCACTAGGGGCCATATCTCATGTTGGACCCCTTGTAGGGCCTTTAAACTGGCCAGA |
| | TAACTTGGGGCCACATTGGGGTCATGATCTGGTAGAGTACGAACAATAATGGGGGGTGGTGCCCCATACA |
| | GAATTTCGAAAGGTGTCAAACCATGTACATATGGTGAGTTCCGGACCCGGAAGATGGCATAGGGTAAGAG |
| | GGTCACCCAGTCCCCGCCAGTCTCGATGGCTAGTTTGGACAAGGTCTCCTTTAGAGTCCGATTCATTCTC |
| | TCTACCTGCCCTGAGCTCTGGGGATTATATTCACAATGTAACTTCCAATTGATCCCTATCGCTCGGGCTA |
| | GTCCTTGTAGGACGTTACTGATGAAAGCTGGGCCGTTATCGGAGCCTAAAACCTCAGGAACCCCATATCT |
| | GGGAATAATTTCTTCTAGTAATGCCTTAGCAACCACTTGGGCAGTCTCCCGTTTCGTGGGGAAGGCTTCC |
| | ACCCAGCCCGAAAATGTGTCAACCATTACTAGCAAGTACTTATACCCACACCTCCCAGGCTTTACCTCAG |
| | TAAAATCCACTTCCCAACTCCGTCCCGGCGCTCTTCCCCGTACCCTCGTACCTGTATGTTGGGGTCCTTT |
| | TCTACTGGGTCTCATAGCCTGGCACCCAATGCACTGATCTACAATCTCTTGAATCTGAGCCGCTTGTCGG |
| | GGAAACCGGAGGCGGGCGGACTCGAGAATTGTCAGCAACTTCTTTTTTCCTAAGTGGGTGGCTTGATGCA |
| | GGTTGGAGAGAAGAAACAGTCCTAGCTGTGCCGGCAGTATCAATCTTCCTTCTGTATCCCGATGCCACCC |
| | CTGCTGATCAGATTCCGGGCAGTGGTGGTTCTGGATCCATCGCAGGTCTTCTGGAGTGTAGTCAGGTCGC |
| | GGGGGCAGGCGAGGGAGCTCAGGTGTGGGCAGGGTGAGTGCTAAAGCTGATGAAGCTACTGCCACTGCCT |
| | TGGCGGCTTCATCCGCTCGCCGGTTTCCTTTAGCTTCCGGGGTCTGGGCAGACTGGTGCCCAGGGATGTG |
| | GACAACTGCGACTGCCCGGGGCATTTGTACAGCCATCAGCAGTCTTCGTACCTCAGGAAGATTGCGCAGA |
| | GTCTTTCCTTCCGCTGTAACAAAGCCTCTTTCCCGGTAGATAGCGCCATGCACATGGACAGTGCCAAAGG |
| | CGTAGCGGCTATCGGTGTAGACAGTCACTCGTCTCCCTTCGGCCCGTTCCAGCGCTTCCGCCAGCGCGAT |
| | CAGTTCGGCCTTCTGTGCTGATGTCCCCGGGGAAAGCGAGGCACTCCAAATGATGTTTCCCCCTTGGTCT |
| | ACCACCGCTGCGCCTGCCCTCCGCACACCATCTATAACGAAGCTGCTTCCATCAGTGTACCATACCAACT |
| | CACTGTTGGGTAGTGCGGTGTCCTGGAGGTCGGGGCGCACCTGGGTGACTTCTGCCATGATCTCTTGGCA |
| | GTCATGCAGGGGAGCTCTCAGATCCGGGGTCGGCAGCAGGGTGGCTGGATTCAGAGCGGTGGGTTCAGCG |
| | AAGATGATCCGGGGTGCATCTAGCAAGAGTCCTTGGTAATGTGTTAGTCGGGCATTAGTCATCCACCTAC |
| | CAGGGGGATATTTCAGGACCCCCTCGATCGCATGGGGGGTTACTACCTTCAGATGTTGCCCAAAAGTGAG |
| | TTTATCAGCATCCTTCACCATTAGGGCTACTGCCGCAATGATCCTCAAGCACGGGGGCCATCCTGCTGCA |
| | ACTGGATCTAGCTTCTTGGATAAATAGGCAACCGGGCGTTTCCAGGGCCCCAGACGCTGCATTAGCACCC |
| | CTTTCGCTATTCCCCTCCTCTCATCAACAAAGAGAGTGAAGGGCTTCAGGGGGTCTGGCAATGCCAGAGC |
| | CGGGGCTCTTAGGAGAGCGACCTTGAGTTCATCGTAGGCCTTCTGTTGGTCTGACCCCCAGGCCCAAGGG |
| | ACCTTATCCTTGGTTGCCTCATACAGAGGTTTTGCTATTTCAGCATACCCCAAAATCCACAGCCGGCAGT |
| | AGCCTGTCGTCCCTAAAAACTCACGGACCTCTCGTGCTGAGGTCGGGACTGGAAGTCTAAGAATAGTCTC |
| | TTTCATGGCCTCTGTCAGCCATCTGGCTCCTTTTTTTAGTTTATACCCCAGGTAGGTGACTGTTTGCCTG |
| | CATATTTGAGCCTTCTTTGCACTGGCCCGATAGCCCAACTGCCCCAGCTCCTGGAGGAGGTCTCCAGTGG |
| | CCTGTCGGCATTCAGCTTCGGAGGGGGCTGCCAGAAGCAAGTCATCTACGTACTGCAGGAGCGTAACTGA |
| | ATTATGGCTCTGGCGAAACGAGTCCAAATCCTGATTTAGGGCTTCATTAAACAGAGTTGGAGAGTTTTTG |
| | AAGCCTTGCGGTAGTCTAGTCCAGGTCAGCTGCCCGGGGGTTCCCGTATTGCCATCATTCCATTCGAAAG |
| | CAAAAATGTGTTGGCTGCTGGGTGCCAGGGCTATGCTAAAAAACCCATCCTTTAGGTCTAAGGTAGTATA |
| | CCAGACATGTGAAGGGGGCAAGTGACTTAGTAAGGTATAAGGGTTGGGGACCGTGGGATGGATGTCTTCA |
| | ACCCTCTTATTTACTTCCCTCAAGTCCTGGACTGGCCTATAATCTTTTCCCCCCGGTTTCTTAACGGGGA |
| | GAAGTGGGGTGTTCCAGGCAGAATGGCAAGGTTTCAGTATTCCAGCTTCCAGTAAACGGTTAATGTGCGG |
| | GGCAATCCCTTTCCGCGCCTCTGCAGACATGGGGTACTGGCGGATCCGGATAGGCTGGGCTGAGGCTTTA |
| | AGTTCCACCACTACTGGTGCTCGGCGGGCCGCCCGGCCCACACCCGCTATTTTTGCCCACGCCTGAGGGT |
| | ATGTTTTAAGCCAATAATCCATATCACGGGGCCATTCTGTAGAGGGAGGGTTGTAGGGGTTGTCCTGCAG |
| | GGCGAACAGGCGATGTTCATCCACAAGAGACAGGGTCAAAATGTGGAGGGGCTGTCCTTGGCCATCCAAT |
| | AGCTTAATGCCATCCGGCTCAAAATGGATCTGAGCCCTGATCTTAGTCAGGAGATCGCGCCCCAATAAGG |
| | GGGCAGGGCATTCAGGGATAACTAGGAAGGAGTGGGTCACTTGGTGGCGGCCTAAGTCTACTTGGCGCCT |
| | ACTAGTCCACCGATAAGCCTTGGACCCAGTTGCCCCTTGCACCAAACTGGTTTTCTGAGATAAGGGCTCT |
| | GTGGGCTTATTCAAAACTGAGTACTGGGCTCCTGTGTCTACCAGGAATCCTACTGGCTTCCCCTCCACAT |
| | ACGCAGTTACCCAAGACTCGGGGAGGGGATCCGAGTCCCGTCTCCCCTAGTCACTCTCCATCCCCGCCAG |
| | CAGGACCCGTGCGTCTTGCCCTGTTTGGCCCTGGCGCTTGGGGCACTCCCTCTTCCAATGTCCATACTCT |
| | TTGCAGTTTGCACACTGTCCCCTATCCAGTCGGGGCCGCGGTCTCCACGGTCGGGCTGGTCCGGCCGGAC |
| | TCGGTCCCACCCTGACTGTGCTTTGCACGCCTGCCAACAAGATCTTGGCCATCTCCCTCTGCTGCCTCCT |
| | GTTCTCCCTACTCTGATGCTCTCTGTCTTCCTTCCTGATTCGTTCCTGTAATTCCTGATTTTCTTTTCTA |
| | ATTCTATCCTCCCTTTCTTCGGGAGTCTCTCGAGTGTTGAAGACTCTCTCCGCTACTTTCATTAAATCCC |
| | GGATAGACATTTCTCCCAGTCCCTCCTGTTTGTACAATTTCTTCCTAATATCTGGGGCAGCCTGGTTTAT |
| | AAAGGACATAATTACAGCCGACTGGTTTTCCTCTGCCAGGGGGTCCAACGGGGTGTACTGTCTGTAAGCA |
| | TCATAGAGGCGTTCTAAAAACAGGGCCGGGCTTTCATTATCCCCTTGCATTATAGCTTTTACCTTGGCCA |
| | AATTGGTGGGGCGGCGTGCCGCCGCTCGGAGACCTGCCATAAGAGTCTGGCGGTAGACTCGGAGACGCTC |
| | CCTACCTTCTGCGTTCCCAAAGTCCCAATCCGGTCTATTCAGGGGAAAGCGCTCGTCTATCAGGTTCGGC |
| | AAGGTCGTCGGTCTTCCGTTGTCGCCGGGGACATTTTTTCTGGCCTCGGTGAGGATTCGCTCGCGCTCCT |
| | CGGTGGTGAATAAGGTCTTAAGAAGCTGCTGGCAATCATCCCAAGTGGGACTGTGTGTGTGCATGACAGA |
| | CTCGAACAGGTCAGTTAGGCCTTTCGGGTCCTCAGAAAAAGGAGGGTTTTGAGCCTTCCAGTTGTACAGA |
| | TCACTGCTAGAAAAAGGCCAGTACTGGTATGCTCGCTCCCCATCTGGACCAGTTCCTCCTAGTGCTCGCA |
| | CAGGGAGAATCGGCGCCCCAGCGGAGGTGGAGGAGGACGGTTCCTCCTCTGGGGTCTCTTCCCGGCGTCT |
| | GCGAGGCCTCAATCCCCTCGCCGGCCCTTGAGCTGGGGCAGGGGAACCCGGGGGAGGGGGAGCCATCGGG |
| | GAGGCGGTAGAGTGAGGCAGCTCGGAAGGAGCGGCAGCCTCAGGCGGGAGCGGCGCCATCGGGCTGGGCG |
| | CGCGCCGCGGCTGGAGCGGCGCCACCGGCGCGTAAGGAGGGGGGGTCTCTTCCAGATCTAGGTCTATCAG |
| | GGAGGGGTATATGTCTGACCCCTCCTGAAGGATGGGTCCCTGGGTCGGCTTCTCCGGGGGTTTCGGGCCG |
| | GCCGTGGGCACTGCCGACTGGCGGGAGGGTCCCGAAACGGTCAGGACTTTAAGGGGGAGGGGGGGATCTT |
| | CTGGCTTGTCGGGGATAAAGGGCTTAAGCCAGGAGGGAGGACTCTCTACTAAGGCTTGCCACATCAAAAT |
| | ATAGGGATATTGGTCCGGATGGCGCCGATTAATAATATCTCGGACCTTTTTAATAATGTCTAGGGAAAAA |
| | GTTCCCTGGGGGGGCCAGCCCACATTAAAAGTAGGCCATTCTGCAGAGCAGAATGTATCAAACTTACCTT |
| | TCTTCACTTCCACACCATGATTACGAGCCTTGGCGCGGATTTCAGGAAAGTGGTTCAGGAGCAGGGTCTT |
| | AGGTGTTACCTGAACCTGTCCCATAATTGTCACAAAGAGAAACCAAGAAAAGGCAAAAGAAAGGACAAAA |
| | GACACAGTGCCAGCAAATACACAACTTCGCACAGGACTCTTCAACACCCACCGGCCGGTCAACCACACCA |
| | CATCCACAGGCGCCGTTTCAATCACACCAGTCTCACCACGCTCAAGATCCTTACCTAGGGCCCGTCCAAA |
| | CGGCGTCCACTGTGGACGTCGCTGGGCCACCTTCTCGTCGGGGACGTCTCCCACGACTTCAAGTAACGAA |
| | GCCTCCAGGGTCGTAACCTGCACTTTCCTTCCCGTGAGAATTCTCAACTGGGACCGGGCAGAGACCTGTT |
| | TCAGTCTCTCCGGTCGAGGACCTGTTTCAGTCCTCCCCTGTTTGGGACCGGGCAGAGACCTGTTTCAGTC |
| | TCTCCGGTCGAGGACCTGTTTCAGTCCTCCCCTGTTTGGGACCGGGCAGAGACCTGTTTCAGTCTCTCCG |
| | GTCGAGGACCTGTTTCAGTCCTCCCCTATTGGAGGTGGCCAAACCTCCTTCCGCGGTTCCCTATGTAAAC |
| | CTCGGTATCGGGAGTTGTCTGTTCCCCTGAGGGGGGGCGTCCCGGGCGAGCCCCCAAATGTTAAGATCCG |
| | GAATTGTCCCCCTCAGACAGACAGAGAACCCGCACAATAATGCAAGTCACACAGCGAGGTTTATTACCAG |
| | CCGGCTGGGGTCCCCGTCTCTGCCCGACGCAGCGGGTTTTTAACAAGGACCCCAAACACTTAAAGCCAAG |
| | GGGTTATATAGTATTTTCAAGGCCTTCCATAGCTTCTGAGAGTACAAGATAAACTTACAGGACTTACATA |
| | GTTTCAAAGAGTATAAGATTTACTACAGGACAAGGAGACCAGGAGTATAAGATAAGCTACAGGACTTCTA |
| | GTCGCCATGCTGCAACTGCCCACATCCTGGAATTTTATAATTATGTTGTTTCAGGCTAGGGGCTGTTAAC |
| | CCTGACCTGAAGATAGCAGTTCTCATGCTAACAGTCTCTTACATGCTAACAGTCTCTTACAGTAAGAAGT |
| | TCCAAAGCCTGTGGTGGCAGTAAGTGAATTTCTTCCTTTTCAATAGACTATGAAGGAGGGACATTGCATT |
| | TGAACTCAGTCCATGAGTCATGATGCTCTTTATGTCCATTAAAAGGATTAACTTTCTCTCTATTCACTAT |
| | TTCTTTCACACTATTGTATAGGGTAACGTGTTTGGGGAGAAAAATCAATAAAAATGCTTAAAATAAAAGT |
| | TTCCATGCTCATAAGGTTTTTATCTTCCATTATAGGAAAATGAATCTATATGGAAGGGTACATTTTCTGA |
| | TGATGTTTTGTAAGAAGCATTATTCTATCAATCTATTAAAATATATTGATGCACTTTCC |
| 7 | Part of RFe-MD-2 sequence with Columbid/Falconid DNA homology |
| | TCCTCGTTAGTATAGTGGTGAGTATCCCCGCCTGTCACGCGGGAGACCGGGGTTCGATTCCCCGACGGGG |
| | AGGCAGv |
| 8 | Protein sequence of RFe-MD-2 fragment that shows homology with |
| and | Columbid and Falconid herpesvirus homologous with hypothetical |
| 356 | proteins CoHVHLJ_080/FaH\HV1S18_80 of the Columbid or Falconid |
| | herpesvirus PRRGIEPRSPA*QAGILTTILTRM |
| 9 | Part of RFe-MD-2 sequence with Sindbis virus (hairpin) homology |
| | TATAGTGGTGAGTATCCCCGCCTGTCACGCGGGAGACCGGGGTTCGATTCCCCGACGGGGAGGCA |
| 10 | Part of RFe-V-MD3 sequence with Human herpesvirus 4 isolate HKNPC6 |
| | homology |
| | TTCATCCATGTCACAAATGACAAGATTTTGTTTTTTATAGCTGAGTAATATTCCATTGTATACATATACC |
| | ACATCTTCTTTGTGTATTCGTCTGTCAGTGAACTTTGGTTACTTCCATATCTTGGCTGTTGTAAATAATG |
| | CTGCAGTGAACATAGGGGTGTGTATATCTTTTCGAATTAGTATTTTGGATTTTTTTCAGATAAATACCCA |
| | GAAGTGGAATTGCTGGGTCATATGGTAATTCTATTTTTAATTTTTTGAGGGACCTCCATACTGTTTTCCG |
| | TAGTGGCTGCACCAATTTACAAGGTGCTTTTCTCTACATCCTTGCCAACACTTGTTGTTTATTGATTTAT |
| | TGATGATGGCCATTCTGACACGTGTGACATGATAGCTCATTGTGGTTTTAATTTGCATGTCCCTGATGAT |
| | TAGTGACATTGAGTATTTTTTCATATGTCTATTGGCCATCTCTGTGTCCTCTGGAGAAATGTCTGTTCAG |
| | GTCCTCTGCCCATTTTTTAAATCAGATTGTTTCGTTTTGTGTGTTAAGTTGTATGAGTTCCTTATATATT |
| | TTGGATATTAAACCCTTATTGGCATCTTCTCCCATTCAGCAGGTTATCATTTTGTTTTGCTAATGGCATC |
| | CTTCACTGTGCAAAAACTGTTTAGTTTGATGTAGTCCCATTTGTTTATTTTTTTTCTTTTGTTTCCCCTG |
| | CCAGAGGAAACATATTCAAAGAAATACTACTAAAAGAGATGTAAAAGCGTTTACTGCCTATATTTTCTTC |
| | TAGGAGTTTTACGGTTTTGGGTCTTAAATTTAACTCCTTAATCCATTTTTAGTTTATTCTTATATGTATA |
| | CAGTGATCCAGTTTCATTCTTTTGCATGTATCTGTCTATAGTTTTTCCAACACCATTTACTGAAGAGACT |
| | GTCTTTACCCAATTATATATTTTTGCCTCCTGTCATAGATTAATTGACCATGTGGGCATGGGTTTATTTC |
| | TGGGTTCTGTTCCATTGATTTATGTGTCTGTTTTTATGTCAGTACCATGATGTTTTGATTACTATGGTCT |
| | AGTAGTATAGTTTGATATCAAGTAGCATGATACCTCCAGCTTTGTTCTTCTTTATCAAGATCGCTTTAGC |
| | TATCTGGGGTCTGTTGTGGGGTCTACAAATTTTAGGGTTACTTGTTCTGGTTCTGTGAAAATGCCATTGG |
| | TATTTTGATAGGAATTGCATTGAATCTGTAGATTGATTTGGGTAGTATGAACATTTTAATGATGTTAATT |
| | CTTTCTATTCACAAACATAGTATATGCTTCCATTTATTAGTATCTTAACTTTCATTCTTCAGTGTCTTAC |
| | AGTTTTCCAAGCACAGGTCTTTTACTTCCTTAAATTCATTCCTAGGTATTTTATTCTATTTAATGCAATT |
| | TTAAATGGGATTGTTTTCTTAATCTCTCTTTCTGATAGTTTGTTATTGGTGTATAAAAATGCAACCAATT |
| | TCTGAATATTAATTTTGTGTCCTGATACTTTACTGAATTCATTTATTAGTTCTAATTGTTTTTTTGGTGG |
| | AATCTTAAGGTTCTCTCTATATAGTATCATGTCATCTGTGAATAATGACAATTTTACTTCTTCCTTTTCA |
| | ATTTGGATGGCTTTTATTTCTAGTCTGACTGCTGTGGCTGGGACTTCTAGTACTATGTTGAATAAAAGTG |
| | AAAGTGGCTTGTTCCTGATCTTAAAGGAAAAGCTTTCAGCTCTTCACTACTGAGTATGATGTTAGCTGTG |
| | GGTTTGTCCTATATGGCCTTTATTATGTTGAGGTATTTTCCCTCTATTCCCAATTTGCTGAGAGTTTTTA |
| | TCATAAATAGATGTTGGATTTTGTCAAATGCTTTTTCTGCATCTATTGATATGATCATATGATTTTTATC |
| | TTTCATTTTGTTTATATAGTTTATCACATTAATTGATTTGCAAATATTGAACCAACCTTGCATGCCAGGA |
| | ATAAATCCCACTTAATCATGGTGTATGAACTTTTTAATGTACTGCTGAATTTGGTTTGCTAATACTTTGT |
| | TGAGGATTTTTGCATCTATGTTGTTCATCAGGGATGTTGGGCATTTTTTTTTTTTTTTTGTATTGTCTCT |
| | GGTTTTGGTATCAGGCTAATGCTGGCCTTGTAAATGAGTT |
| 11 | Part of RFe-V-MD3 sequence with Human herpesvirus 4 isolate |
| | HKD40homologyTAAATACCCAGAAGTGGAATTGCTGGGTCATATGGTAATTCTATTTTTTAATTTTTTG |
| | AGGGACCTCCATACTGTTTTCCGTAGTGGCTGCACCAATTTACAAGGTGCTTTTCTCTACATCCTTGCCA |
| | ACACTTGTTGTTTATTGATTTATTGATGATGGCCATTCTGACACGTGTGACATGATAGCTCATTGTGGTT |
| | TTAATTTGCATGTCCCTGATGATTAGTGACATTGAGTATTTTTTCATATGTCTATTGGCCATCTCTGTGT |
| | CCTCTGGAGAAATGTCTGTTCAGGTCCTCTGCCCATTTTTTAAATCAGATTGTTTCGTTTTGTGTGTTAA |
| | GTTGTATGAGTTCCTTATATATTTTGGATATTAAACCCTTATTGGCATCTTCTCCCATTCAGCAGGTTAT |
| | CATTTTGTTTTGCTAATGGCATCCTTCACTGTGCAAAAACTGTTTAGTTTGATGTAGTCCCATTTGTTTA |
| | TTTTTTTTCTTTTGTTTCCCCTGCCAGAGGAAACATATTCAAAGAAATACTACTAAAAGAGATGTAAAAG |
| | CGTTTACTGCCTATATTTTCTTCTAGGAGTTTTACGGTTTTGGGTCTTAAATTTAACTCCTTAATCCATT |
| | TTTAGTTTATTCTTATATGTATACAGTGATCCAGTTTCATTCTTTTGCATGTATCTGTCTATAGTTTTTC |
| | CAACACCATTTACTGAAGAGACTGTCTTTACCCAATTATATATTTTTGCCTCCTGTCATAGATTAATTGA |
| | CCATGTGGGCATGGGTTTATTTCTGGGTTCTGTTCCATTGATTTATGTGTCTGTTTTTATGTCAGTACCA |
| | TGATGTTTTGATTACTATGGTCTAGTAGTATAGTTTGATATCAAGTAGCATGATACCTCCAGCTTTGTTC |
| | TTCTTTATCAAGATCGCTTTAGCTATCTGGGGTCTGTTGTGGGGTCTACAAATTTTAGGGTTACTTGTTC |
| | TGGTTCTGTGAAAATGCCATTGGTATTTTGATAGGAATTGCATTGAATCTGTAGATTGATTTGGGTAGTA |
| | TGAACATTTTAATGATGTTAATTCTTTCTATTCACAAACATAGTATATGCTTCCATTTATTAGTATCTTA |
| | ACTTTCATTCTTCAGTGTCTTACAGTTTTCCAAGCACAGGTCTTTTACTTCCTTAAATTCATTCCTAGGT |
| | ATTTTATTCTATTTAATGCAATTTTAAATGGGATTGTTTTCTTAATCTCTCTTTCTGATAGTTTGTTATT |
| | GGTGTATAAAAATGCAACCAATTTCTGAATATTAATTTTGTGTCCTGATACTTTACTGAATTCATTTATT |
| | AGTTCTAATTGTTTTTTTGGTGGAATCTTAAGGTTCTCTCTATATAGTATCATGTCATCTGTGAATAATG |
| | ACAATTTTACTTCTTCCTTTTCAATTTGGATGGCTTTTATTTCTAGTCTGACTGCTGTGGCTGGGACTTC |
| | TAGTACTATGTTGAATAAAAGTGAAAGTGGCTTGTTCCTGATCTTAAAGGAAAAGCTTTCAGCTCTTCAC |
| | TACTGAGTATGATGTTAGCTGTGGGTTTGTCCTATATGGCCTTTATTATGTTGAGGTATTTTCCCTCTAT |
| | TCCCAATTTGCTGAGAGTTTTTATCATAAATAGATGTTGGATTTTGTCAAATGCTTTTTCTGCATCTATT |
| | GATATGATCATATGATTTTTATCTTTCATTTTGTTTATATAGTTTATCACATTAATTGATTTGCAAATAT |
| | TGAACCAACCTTGCATGCCAGGAATAAATCCCACTTAATCATGGTGTATGAACTTTTTAATGTACTGCTG |
| | AATTTGGTTTGCTAATACTTTGTTGAGGATTTTTGCATCTATGTTGTTCATCAGGGATGTTGGGCATTTT |
| | TTTTTTTTTTTTGTATTGTCTCTGGTTTTGGTATCAGGCTAATGCTGGCCTTGTAAATGAGTTTGAGAGC |
| | CTTCCCTCCTTTTCAGTTTTTTGGAATGTTTGGTAAAATTTACCTGTGAAGTCATTTGGTTCAGGGCTTT |
| | TGTTTGTTGGGAGTTTTTTGATTACTGATTCGATTTTGTTAGCAGTTACTGGTCTGTTCAGATTTTCTGT |
| | TACTGATTCAGCCTTAATTTTCTGCTGATTCAAGCCTTGGAAGATTGTATGTGTCTAGCGATTTATCCAT |
| | CTCTTCCAGTTTGTCCAATTTGTCAGCATATAGTTGTTCTAGTGTTTCCTTATACTTCTTTGTATACCTG |
| | TGGTGTCAGTTGTCGTATCTCTTTCATTTCTGATTTTATTTTGGCCCTCTCTCTTTTCTTCTTGAGTCTG |
| | GCTAAAGGTTTATCAAT |
| 12 | Part of RFe-V-MD3 sequence with Human respiratory syncytial virus |
| | (Kilifi isolate) |
| | homologyTTTTCTTCTAGGAGTTTTACGGTTTTGGGTCTTAAATTTAACTCCTTAATCCATTTTTAGTT |
| | TATTCTTATATGTATACAGTGATCCAGTTTCATTCTTTTGCATGTATCTGTCTATAGTTTTTCCAACACC |
| | ATTTACTGAAGAGACTGTCTTTACCCAATTATATATTTTTGCCTCCTGTCATAGATTAATTGACCATGTG |
| | GGCATGGGTTTATTTCTGGGTTCTGTTCCATTGATTTATGTGTCTGTTTTTATGTCAGTACCATGATGTT |
| | TTGATTACTATGGTCTAGTAGTATAGTTTGATATCAAGTAGCATGATACCTCCAGCTTTGTTCTTCTTTA |
| | TCAAGATCGCTTTAGCTATCTGGGGTCTGTTGTGGGGTCTACAAATTTTAGGGTTACTTGTTCTGGTTCT |
| | GTGAAAATGCCATTGGTATTTTGATAGGAATTGCATTGAATCTGTAGATTGATTTGGGTAGTATGAACAT |
| | TTTAATGATGTTAATTCTTTCTATTCACAAACATAGTATATGCTTCCATTTATTAGTATCTTAACTTTCA |
| | TTCTTCAGTGTCTTACAGTTTTCCAAGCACAGGTCTTTTACTTCCTTAAATTCATTCCTAGGTATTTTAT |
| | TCTATTTAATGCAATTTTAAATGGGATTGTTTTCTTAATCT |
| 13 | Part of RFe-V-MD3 sequence with SARS-CoV-2 |
| | homologyAGGTTCATCCATGTCACAAATGACAAGATTTTGTTTTTTATAGCTGAGTAATATTCCATTGT |
| | ATACATATACCACATCTTCTTTGTGTATTCGTCTGTCAGTGAACTTTGGTTACTTCCATATCTTGGCTGT |
| | TGTAAATAATGCTGCAGTGAACATAGGGGTGTGTATATCTTTTCGAATTAGTATTTTGGATTTTTTTCAG |
| | ATAAATACCCAGAAGTGGAATTGCTGGGTCATATGGTAATTCTATTTTTAATTTTTTGAGGGACCTCCAT |
| | ACTGTTTTCCGTAGTGGCTGCACCAATTTACAAGGTGCTTTTCTCTACATCCTTGCCAACACTTGTTGTT |
| | TATTGATTTATTGATGATGGCCATTCTGACACGTGTGACATGATAGCTCATTGTGGTTTTAATTTGCATG |
| | TCCCTGATGATTAGTGACATTGAGTATTTTTTCATATGTCTATTGGCCATCTCTGTGTCCTCTGGAGAAA |
| | TGTCTGTTCAGGTCCTCTGCCCATTTTTTAAATCAGATTGTTTCGTTTTGTGTGTTAAGTTGTATGAGTT |
| | CCTTATATATT |
| 14 | Part of RFe-V-MD3 with RNA-dependent DNA polymerase of Erythrocytic |
| and | necrosis virus homology |
| 358 | PTSLMNNIDAKILNKVLANQIQQYIKKFIHHD*VGFIPGMQGWFNICKSINVINYINKMKDKNHMIISID |
| | AEKAFDKIQHLFMIKTLSKLGIEGKYLNIIKAI |
| 15 | Part of RFe-V-MD3 with RNA-dependent DNA polymerase of Lymphocystis |
| and | disease virus homology |
| 357- | MTSQVNFTKHSKKLKRREGSQTHLQGQH*PDTKTRDNTkkkkKC-PTSLMNNIDAKILNK |
| 359 | VLANQIQQYIKKFIHHD*VGFIPGMQGWFNICKSINVINYINKMKDKNHMIISIDAEKAFDKIQHLFMIK |
| | TLSKLGIEGKYLNIIKAI*DKPTANIILSSEELKAFPLRSG |
| 16 | Prediction of a potential new spike protein sequence (RFe-SP2) |
| | (M)FVVVVVVVFPFRLPHHSGVSYCFSVLCRTQLPGPCWYYEPCAPPSGSRLLVGPLGHSQHFMSVLAGC |
| | RSLTLATSRSWQHMVAHGSPWQPSSRESRCSQSLRMQRTGPRGNRTSMALQPPEPPCEGCEGWLTKVFNF |
| | DEIQLFSFVACALMLYLRRHCLIQGHGDLHLCFLQVLLHLFVYLSISSFLYMVGRVSKFILLHVDIQLSH |
| | HLLKRLFSSIELSWHIYQKSIDHGFILDSSIPLIYMSVFMPVPHSLDYCSFAVSFIRKYESSHFVPFFQD |
| | CFGYSGFLAFPCKITQLVNFCRKIKVAKIFVGFAVNNSNGVLLFQNVFSTKAGFKFYLGLFLSTMFCCFP |
| | RTSVILLCIYSLISFCYHKWCCFLISFSDCSLLVYRNTTFFFPYPCILKSCIHLLALIVLLGWGLGVDSL |
| | AFSKGHVIKIVLVLLHFQSFFLFHFLTNLARTSGRMLNSSGESRHPCLVTDLRKKASSLSPLSMVLAVGF |
| | SMLFIRLRKFPPTFASVFFSFPSNHMIPIWHTGKQMTNVEMCSRYIKLEMRPRYPDSHSTVLSTILYYFL |
| | WSPAATFRDQKKVHPFHLLIKKVSSITVGFVSGFVPLFPWNLKVPGTEVLVVSRKSQFRQIPLEPLLCAR |
| | QCAQYSSPQRDCGGGDETSYKKIIRRDLIVGNRRQLPEAEPFVNKKVFVEETGMNRALKEGQLTCVCGST |
| | LGRIFDRKLKNVLLFNFYMKSLKPITITRDMQSKNNNRVHIRSGNILFFSDLFFGLRLKLSKSAFSFCPE |
| | NIGSICFIPPIENFFRQPVSDVQVLFFVYCSMLLLQVFSVLRARLLILHTDHMWLKTTGSHHEQVRAAIW |
| | ELTIHHTFIDYPLARYMNDRGSIQADMQISYYNLIDNPREVFFCHSLDVFMLHPIETCFRGIIIVPSTDI |
| | FEHLILRSKSRANIDVELCSMQSPSPIVLLLIISEFSVSSGFERPPEPLFHNNSTLSSLIPNSTQKIKGE |
| | RKVCSTDKNFSIRISTRCDTIQTLLLSAIQWKGRDIQYKKLHVKPCVTSFNLFGAVSWVDTLERRCVLQC |
| | AVNHPLSQCSNIASGIQQFLSTKDIMVCPHPPYPDLAPCDCWLFPKEKMTTKGKLFELIQDIKAATTVQL |
| | KTLIKEDFQNCFRKWQKQRDTCVQSKGEYFEENWCVFYCNKFFITFTVFFLSHLIQKKTSRRKLLHFVPP |
| | QLQVVTGSAEYNGHMEKQFSWKFWMFTKNVFENKVIYHHHHLLCRNKTQSEVPWKKSSGWKIPALSPLIT |
| | SCDFSHFSLNCHPHDAPSPRVFHDNSVLLDEAFWWGASESPSRARVCVCVCVSLYSSEQFYRTWRRHGQS |
| | TQRECSLIMNYVKNISPGPVIMVPYDVHSTFMNIEAHLFPMKIRKLGEKIKRTHSLTPNKYWHDGFGFSL |
| | MTLILRVLALILYSILSAQILKLTGWVRIPAAVYQGVVPWAHYVTSLPFSHLKVEVLIVPASHYCENSIC |
| | NTTMPGTSVLTSIYMPQSMAEFCQPPHLICHWTHPTIPGVSGGGXXXXXXXXXXXCX |
| 17 | Prediction of a potential new spike protein sequence (RFe-SP1) |
| | (M)YCSISQLELCWRLTVTGTLQTFTGLDSSLKVFDVNLIRPGHCVFRNSSPSFYNPCFKSTECISVMYH |
| | FTDFKSVRNLKRYSGVLTSSLSFATRLGLELSLPVGSRSERDIIGGICWPTEVHLFPFCHQSFTGAQRLF |
| | RHLLMGSLLISRIRPLKKEFCISVHAQVVRSINVYHQHAFPAAVRNHEPSFLKLCDRLSQYVCLIPTALA |
| | SGGVTSKHQEPGVRTKTERNCFNNLESAVLCRNVFDQSVKNKCKWTVVISFEKFLKWVKVMTSCLYCKLR |
| | PNFWIKRRIPKKGKILHTNIHIIHNHLETANIKSQVPYRINVSPGYVFASMYSTLTILETHVECGCQASL |
| | KNQTWSFLITYCAVPFRSDMCSLVCYCPHLGSSLTLVTFFVSISTGGYDSCLIVYEVQLHSIHSRKSNAY |
| | LIGEYHCGHLQSTPLGKLCTDASASTLQSNFGTLFLEWSSELSSCYPCPECHQNVFLSIFPLSSGKERRH |
| | WGPGEVSREGVPIRLFVCWLKTPSQTVAGVLSCKIHELLEKRSGHFRLKFFTQPFLHFIVNLVNCLSSWY |
| | KFIMNNPSDIKKGQQHRLDPFGLMELFSSWRIGLPFCTLTFCYRIILVHPCFITSDNMANVMLPLQKVLT |
| | NLDSLLFLFTGELLRDHFCTHLPHAKIFSSDFVFLYFYLGLLCFSESANNFDGSFDEFLQFFSSVLLVIG |
| | CPDLSLSVARSLPSEKTFTPLVHCHFILGIILIDLDHVPDFTSTLARFTKKFNVCSLFFKLRHSCDATQK |
| | HTTTIMNAIQQHRHMSTRTQLDLYTKVMKPYRAVCTVLPMTQGGKFSNLILRPHILTNISRFSIQSMFYV |
| | DLLVFYLNFFIVLYRGTVCFSRAHQLQVIALQSRCGGHSSAPSPVAVFQSLVAGGAAHHPMRELNQQPCC |
| | ELTVPTEPLGHPNKTYCLFQKYRKLGEKRKNHSYSQYPKTKTQHCFTFISLKCLLFISLHYTFENQIVSL |
| | AMISPCLLEVFLYCALLNIGILQLLTLNPFSHSISICPAFSHLADKSQINIFYIHYFLIIKSIFPFPYSI |
| | IHVDVFKFKHPCLFELLYSLQISLIASKRKSKSNCVEKTKTKNSKPFGSRIRLVGCRSSKSHSCAISVLL |
| | VPSHFSFFFLISPSECWSFVTTNDYHHLPKLHASRFPGRKELCSFCFYYFPSISTKVLTVTQIRTAKVTC |
| | PTLNQIRPERCNELLCLAVSHHRRAHGYLPRAESGGKRDRTSNETQSCCQNKANGCQELTNNTPSFIEWH |
| | SLIQFQNLASCETTLLPLLPSHLFVFSCLPLSLTRTLEITMDPHRYKTISPSQSFNHLYKVHFAVSNMLT |
| | YFRDDHILRGHYFACHTHIFLNHLHNLILEMCSGIMFILGCFSLLAHSVSFTLALNTHSVPHATLVSHAK |
| | QLFIHFAIMPNSVWHNQGNLFSPLTINLKAFLIPKSQINLKVLLSESQNYFTFERNLAKVHFTFISNKPI |
| | PLNFSIYNDVPLFLVNETKHNTFLKRVKKKKSLKLLGFICYNEVSSASERSSRKNNKTVDITEQLIHELT |
| | TVCLNFNTICHIDPNVPSSASIRALFSHGSESILKSSTHPSADAMGSIPASMALWLKDISVVQSPPLYPP |
| | FHWKLSSLPHKCEPEEIICQEGFLMVNAQILNRVQPFLTQASRTICISGKSHRLKFPPPELCSCCCLSQP |
| | LSGTSWNSWNFQRTETSPIQSELWRYILICIYTDQSELIHNNQSEHMMLTDQNCVIWISHLHKNGPNGNQ |
| | GTNFLCKRPLPLCLGVHFRFFLFTNCSAESLSSFHTSYWKLLLIGRLWSQFTRGPLKLSEMIFKQEDSSR |
| | FEGRGYCPRSSLRSEVCQKREKAVMALVIFQWKQYISLVERVRVSNREDAAGQISPVSHYRQDSTRSSQK |
| | TKWYGCEECIFLFFFFLFFFFFLVCIFLINLLGVAHCHSMKTPGILVSQCLIFDQLFMLLNVHFHRKQMQ |
| | QSSSHLLHSSNPSLYLSDDVYICVCGALDSACQNNTFIDWDTDLLVYVLPLTFHFVSIEYKKQKALGFA |
| 18 | ORF number 1 in reading frame 1 on the direct strand extends |
| | from base 610 to base 837 |
| | TCTCACCTAGCAGGAAGGscadmtctcaggaccatcccatacagcagggtggaggattggtgga |
| | tcaggtacataggcccaatacgtctggtcttcttctgcattgctgagggtcatcaatgtcatca |
| | gcaggtagagggtccacatgtcgcaccaatcgttctggcagccaccgagggccttctgcatcct |
| | gtggaaaaacacaaacatgccctcggccccatatga |
| 19 | Translation of ORF number 1 in reading frame 1 on the direct |
| | strand |
| | SHLAGRXXLRTIPYSRVEDWWIRYIGPIRLVFFCIAEGHQCHQQVEGPHVAPIVLAATEGLLHP |
| | VEKHKHALGPI |
| 20 | ORF number 2 in reading frame 1 on the direct strand extends |
| | from base 3349 to base 3699 |
| | Ccttggatgcccatggtaagagtgctgtggagcgcttttggcatccttctgctgcccctcaggc |
| | tttggtcaaatggaaagacccacttacaggctcttggcaaggcccagatccagtcctcatatgg |
| | ggccgagggcatgtttgtgtttttccacaggatgcagaaggccctcggtggctgccagaacgat |
| | tggtgcgacatgtggaccctctacctgctgatgacattgatgascadmccctctgcatcctgtg |
| | gaaaaacacaaacatgccctcggccccatatgaggactggatctgggccttgccaagagcctgt |
| | aagtgggtctttccatttgaccaaagcctga |
| 21 | Translation of ORF number 2 in reading frame 1 on the direct |
| | strand |
| | PWMPMVRVLWSAFGILLLPLRLWSNGKTHLQALGKAQIQSSYGAEGMFVFFHRMQKALGGCQND |
| | WCDMWTLYLLMTLMXXPSASCGKTQTCPRPHMRTGSGPCQEPVSGSFHLTKA |
| 22 | ORF number 3 in reading frame 1 on the direct strand extends |
| | from base 4186 to base 4740 |
| | agggtccacatgtcgcaccaatcgttctggcagccaccgagggccttctgcatcctgtggaaaa |
| | acacaaacatgccctcggccccatatgaggactggatctgggccttgccaagagcctgtaagtg |
| | ggtctttccatttgaccscadmaatggaaagacccacttacaggcttttggcaaggcccagatc |
| | cagtcctcatatggggccgagggcatgtttgtgtttttccacaggatacagaaggccctcggtg |
| | gctgccagaacgattggtgcgacatgtggaccctctacctgctgatgacattgatgaccctcaa |
| | caatgcagaagaagaccagacgtattgggcctatgscadmATCCCATACAGCAGGGTGGAGGAT |
| | tggtggatcaggtacataggcccaatacgtctggtcttcttctgtattgctgagggtcatcaat |
| | gtcatcagcaggtagagggtccacatgtcgcaccaatcgttctggcagccaccgagggccttct |
| | gtattctgtggaaaaacacaaacatgccctcggccccatatga |
| 23 | Translation of ORF number 3 in reading frame 1 on the direct |
| | strand |
| | RVHMSHQSFWQPPRAFCILWKNTNMPSAPYEDWIWALPRACKWVFPFDXXNGKTHLQAFGKAQI |
| | QSSYGAEGMFVFFHRIQKALGGCQNDWCDMWTLYLLMTLMTLNNAEEDQTYWAYXXIPYSRVED |
| | WWIRYIGPIRLVFFCIAEGHQCHQQVEGPHVAPIVLAATEGLLYSVEKHKHALGPI |
| 24 | ORF number 4 in reading frame 1 on the direct strand extends |
| | from base 4792 to base 6306 |
| | ccaaagccscadmtcgaatttccagagcctctgaaaagatatcagtggcgagtccttccccaag |
| | acatggcaaatagccccaccttgtgtcagaagtttgttagtaaaacaattgataacaccagaaa |
| | acagtttccttctgtgtacattattcattatatggatgacattttattggcttgtaagaaagaa |
| | ggagtattgttagcttgctttgcaaatctgcaaaagaatcttctaacctcgggtcttattattg |
| | cacccgaaaaaatacagagaagtgagccttgttcttacttgggatttcagttgtttgctcagta |
| | tttcactccacaaaaaaaagagcttagaaaagatcatcttaaatctcttaatgattttcaaaag |
| | ttgttgggagatattaattggctgcacccttctttgggattaactactggagatcttaaaccac |
| | tgtttgaaattttaaaaagagattctgatccgacctcccccaggtctcttactgagcctgcacg |
| | gaaggctctctctaaggttgagaaagccattcagcaacagcatgtttcctttttagattattct |
| | aaacctctatatgtgtatattttagataccaaacacacgcccacggcggtgttatggcaagaag |
| | ggccacttagatggatacacctccacgtggctgctcaaaagaatcttactccttattatgaact |
| | tgtggccagtttaattcaggagagtcgcttagaagctcgaaaatattatggaaaggagccagat |
| | tctattgttatcccttttacaaaaatgcagattcaaggcctgatgcagtttacaaacagttttc |
| | ctatcgccttggctcattttgcggggactttggataatcattatcctaagcataaattgcttca |
| | attttttcaacatcatgatccaatttttccttcaattgtgtcccatgctcctcttcctgctgta |
| | cctaatgtttttactgatggatctagcaatggtgtagctgtctatgcactcaatgaaaaagtca |
| | ccaagagagtgcagacacctccagcctcagctcaaattgttgagcttcgagcagttcatatggt |
| | attgcttgattttgcttcccagtcttttaatttattctctgacagccattatgtggttcgtgcc |
| | gtcagaaatttagaaacagtaccttttattagcaccagtaatcctgttattcaggatctgtttc |
| | ttcagatacaacaagccattcagctgcgctgtaacaaattttatattggccatattagagctca |
| | ctctaatcttccaggccctttagcctcaggaaatcaaactgcagattctgccacacagctcatt |
| | gttttaactcaaatagaaaaggcacaaaaggctcttagcttccaccatcaaaacaaccagagct |
| | taagactgcaatatactataactagagaaacagcacgccagatagtaaaacaatgcccagattg |
| | ttcgcatttacagcctgtgcctcattatggagtcaacccttga |
| 25 | Translation of ORF number 4 in reading frame 1 on the direct |
| | strand |
| | PKPXXEFPEPLKRYQWRVLPQDMANSPTLCQKFVSKTIDNTRKQFPSVYIIHYMDDILLACKKE |
| | GVLLACFANLQKNLLTSGLIIAPEKIQRSEPCSYLGFQLFAQYFTPQKKELRKDHLKSLNDFQK |
| | LLGDINWLHPSLGLTTGDLKPLFEILKRDSDPTSPRSLTEPARKALSKVEKAIQQQHVSFLDYS |
| | KPLYVYILDTKHTPTAVLWQEGPLRWIHLHVAAQKNLTPYYELVASLIQESRLEARKYYGKEPD |
| | SIVIPFTKMQIQGLMQFTNSFPIALAHFAGTLDNHYPKHKLLQFFQHHDPIFPSIVSHAPLPAV |
| | PNVFTDGSSNGVAVYALNEKVTKRVQTPPASAQIVELRAVHMVLLDFASQSFNLFSDSHYVVRA |
| | VRNLETVPFISTSNPVIQDLFLQIQQAIQLRCNKFYIGHIRAHSNLPGPLASGNQTADSATQLI |
| | VLTQIEKAQKALSFHHQNNQSLRLQYTITRETARQIVKQCPDCSHLQPVPHYGVNP |
| 26 | ORF number 5 in reading frame 1 on the direct strand extends |
| | from base 6307 to base 6987 |
| | ggcctacgtcctaatgatttatggcaaatggatgtaacacatatacctgaatttggaaaattaa |
| | aatatgttcatgtctccatagacacattttctggctttgtcgtggctaccgctcaaactggaga |
| | ggacacatctcatgttattagacattgtcttgctgcttttgctatgattggaacacctaaaaaa |
| | cttaaaacagataatggctcaggttataccagcaaaaaattctctttattttgccagcaattct |
| | cgatcaatcatgttactggcattccttacaatccccaagggcaagggattgttaaacgcactca |
| | tggcacattaaaagtcaatttacagaaaataaaaaagggggagttatatcccctgacgccccat |
| | aattacctgtctcattctctctttatccaaaattttttgaccttggatgcccatggtaagagtg |
| | ctgcggagtgcttttggcatccttctactgccactcaggctttggtcaaatggaaagacccact |
| | tacgggctcttggcaaggcccagatccagtcctcatatggggccgaggacatgtttgtgttttt |
| | ccacaggatgcagaaggccctcggtggctgccagaacgattggtgcgacatgtggaCCCTCTAC |
| | CTGCTGATGACATTGATGACscadmggctttggtcaaataa |
| 27 | Translation of ORF number 5 in reading frame 1 on the direct |
| | strand |
| | GLRPNDLWQMDVTHIPEFGKLKYVHVSIDTFSGFVVATAQTGEDTSHVIRHCLAAFAMIGTPKK |
| | LKTDNGSGYTSKKFSLFCQQFSINHVTGIPYNPQGQGIVKRTHGTLKVNLQKIKKGELYPLTPH |
| | NYLSHSLFIQNFLTLDAHGKSAAECFWHPSTATQALVKWKDPLTGSWQGPDPVLIWGRGHVCVF |
| | PQDAEGPRWLPERLVRHVDPLPADDIDDXXALVK |
| 28 | ORF number 6 in reading frame 1 on the direct strand extends |
| | from base 7282 to base 7590 |
| | TGGACACATAAAACAACATTTGAAAAGTTTTGTAAATCAGGCACTCCCTGCAGTCAGGTGACTG |
| | ATTTACAAGATGGGACTAGAGACTGGTCTAAAAAATCTGTTAATGTATCtgcttgtgttcscad |
| | mgggtcatcaatgtcatcagcaggtagagggtccacatatcgcaccaatcgttctggcagccac |
| | cgagggccctctgtatcctgtggaaaaacacaaacatgccctcggccccatatgaggactggat |
| | ctgggccttgccaagagcctgtaagtgggtctttccatttgaccaaagcctga |
| 29 | Translation of ORF number 6 in reading frame 1 on the direct |
| | strand |
| | WTHKTTFEKFCKSGTPCSQVTDLQDGTRDWSKKSVNVSACVXXGSSMSSAGRGSTYRTNRSGSH |
| | RGPSVSCGKTQTCPRPHMRTGSGPCQEPVSGSFHLTKA |
| 30 | ORF number 7 in reading frame 1 on the direct strand extends |
| | from base 8518 to base 8751 |
| | GGCGTGAgtgtcattgacataatctggaatctcaggaccatcccatacagcagggtggaggatt |
| | ggtggatcaggtacataggcccaatacgtctggtctttttctgcattgttgagggtcatcaatg |
| | tcatcagcaggtagagggtccacatgtcgcaccaatcgttttggcagccaccgagggccctctg |
| | tatcctgtggaaaaacacaaacatgccctcggccccatatga |
| 31 | Translation of ORF number 7 in reading frame 1 on the direct |
| | strand |
| | GVSVIDIIWNLRTIPYSRVEDWWIRYIGPIRLVFFCIVEGHQCHQQVEGPHVAPIVLAATEGPL |
| | YPVEKHKHALGPI |
| 32 | ORF number 8 in reading frame 1 on the direct strand extends |
| | from base 14551 to base 14847 |
| | agggtccatatgtcgcaccaatcgttctggcagccaccgagggccctctgcatcctgtggaaaa |
| | acacaaacatgccctcggccccatatgaggactggatctgggccttgccaagagcctgtaagtg |
| | ggtctttccatttgaccaaagcctgagtggcagtagaaggatgccaaaagcgctctgcagcact |
| | cttaccatgggcatccaaggtcaaaaaattttgaataaagagagaatgagacaggtaattatgg |
| | ggcgtcaggggatacaactcccccttttttattttttgtaa |
| 33 | Translation of ORF number 8 in reading frame 1 on the direct |
| | strand |
| | RVHMSHQSFWQPPRALCILWKNTNMPSAPYEDWIWALPRACKWVFPFDQSLSGSRRMPKALCST |
| | LTMGIQGQKILNKERMRQVIMGRQGIQLPLFYFL |
| 34 | ORF number 9 in reading frame 1 on the direct strand extends |
| | from base 15370 to base 15627 |
| | ctttggatgcctatgttaagagtgcagctgaacgtttctggcatccttctgccgtccctgaggc |
| | tttggtcagaaagaaggatccacttactggatcatggcaaggcccagacccagtcctcatatgg |
| | ggccgagggcatgtttgtgtttttccacaggatgcaaascadmAGGAGAAACAAGAATGGTGGT |
| | GGCTTTATATCGCAGATAGGAAGGAACAGACATTCGTATCTATGCCATATCATGTCTGTACATT |
| | AA |
| 35 | Translation of ORF number 9 in reading frame 1 on the direct |
| | strand |
| | LWMPMLRVQLNVSGILLPSLRLWSERRIHLLDHGKAQTQSSYGAEGMFVFFHRMQXXRRNKNGG |
| | GFISQIGRNRHSYLCHIMSVH |
| 36 | ORF number 10 in reading frame 1 on the direct strand extends |
| | from base 17263 to base 17661 |
| | cattatacccctcaatacctgaacacgtatcttctaagaacaagggccttttacatcagcacaa |
| | tacaattattatattcaggaagtttaacattgatatggtattattgtctaatatgcaatccgta |
| | ttcaaatttcctcaaatactccactaatacccgttacagtctttgtcttgtttttaagttcagg |
| | atccaatcagggatcacacattgcatttggttgccattcctcgttagcacacttcttggccttt |
| | ttctttttaaatttttcatgccattgatatttttgaggcgtccaggcaaggtattttgtaaatt |
| | agcccttaatttgaatttgtctcattggttactcctgattgtattcatcttaaatatttttggc |
| | aaaaatacaacatag |
| 37 | Translation of ORF number 10 in reading frame 1 on the direct |
| | strand |
| | HYTPQYLNTYLLRTRAFYISTIQLLYSGSLTLIWYYCLICNPYSNFLKYSTNTRYSLCLVFKFR |
| | IQSGITHCIWLPFLVSTLLGLFLFKFFMPLIFLRRPGKVFCKLALNLNLSHWLLLIVFILNIFG |
| | KNTT |
| 38 | ORF number 11 in reading frame 1 on the direct strand extends |
| | from base 18964 to base 19221 |
| | ttcagtgctgacactgtctacctggatctgataatatcagatcccacaggtcaagggctcagtc |
| | ccacaggacggctgtcccccccttcagatgccaatcacaagtcgcaggttgtcacctatataca |
| | ccaaatggctataaatcagggtacccgcgactccctccttgggttcagtaatttgccggaatgg |
| | ttcacagaactcaggaaaacacattaccagtttattatgaaagactatgataaaggatatatat |
| | ga |
| 39 | Translation of ORF number 11 in reading frame 1 on the direct |
| | strand |
| | FSADTVYLDLIISDPTGQGLSPTGRLSPPSDANHKSQVVTYIHQMAINQGTRDSLLGFSNLPEW |
| | FTELRKTHYQFIMKDYDKGYI |
| 40 | ORF number 12 in reading frame 1 on the direct strand extends |
| | from base 19894 to base 20241 |
| | aggttagatatagatattttcctattatctcacaGCATTTATCTTAGAAATAAGAACTTGGTTA |
| | GAATGATTGCCTTTCTGGTGAAGTCTATTTTATTTCAACATTTCTTTCATTATTTTATTTTAAA |
| | Ataccaaattaacatgttgtatgccttaaatttgcacaatgttacatgtcaaatacattttttt |
| | tttaaacttttacttattttaagtgtgttttcccaggacccatcagctccaagtcaagtagttt |
| | caatcgagttgtggagggcgcagctcacagtggcccatgtggggattgaaccagcaaccttgtt |
| | gttaagagctcacgctctaaccgactga |
| 41 | Translation of ORF number 12 in reading frame 1 on the direct |
| | strand |
| | RLDIDIFLLSHSIYLRNKNLVRMIAFLVKSILFQHFFHYFILKYQINMLYALNLHNVTCQIHFF |
| | FKLLLILSVFSQDPSAPSQVVSIELWRAQLTVAHVGIEPATLLLRAHALTD |
| 42 | ORF number 13 in reading frame 1 on the direct strand extends |
| | from base 21031 to base 21306 |
| | CATTTTAGAGTATACTCTTTGTGTATGTATCATTTGAAGCACACTCCCATTAGTGTTTACCATT |
| | TTACTTGGGATTTTTATAAAAGTCATTCTATGGTGTTAAAGAGATTGTGCTGCAGTATAGTTTC |
| | ACTGTGTACTGCAGTCCCAAAGGAAAGGGAGCCAGTAAAGACGTGCCGCTTTTTTTCCACAAGA |
| | GTACCATATTTCTTAACGTTGGCTATAAAATTTTACTTCATGAGTCCCGAAGCAGCAAAATACC |
| | TCTTTGAAAGTCACATTTGA |
| 43 | Translation of ORF number 13 in reading frame 1 on the direct |
| | strand |
| | HFRVYSLCMYHLKHTPISVYHFTWDFYKSHSMVLKRLCCSIVSLCTAVPKEREPVKTCRFFSTR |
| | VPYFLTLAIKFYFMSPEAAKYLFESHI |
| 44 | ORF number 14 in reading frame 1 on the direct strand extends |
| | from base 21622 to base 21849 |
| | TGTCTACATTTAATTCTTTGTAGTTGGAAGTTCACGAGGCTAAGCCCGTGCCAGAAAATCACCC |
| | GCAGTGGGATACAGCAGTGGAGGGGGATGAAGACCAGGAGGACAGCGAGGGCTTTGAAGACAGC |
| | TTTgaggaagaggaggaagaagaggaagatgacgaCTAAGCAGTACTGCAAACGGACCACAATA |
| | CTTTCACATTTTCACTGTTTTGGAAGTGTAGAATAA |
| 45 | Translation of ORF number 14 in reading frame 1 on the direct |
| | strand |
| | CLHLILCSWKFTRLSPCQKITRSGIQQWRGMKTRRTARALKTALRKRRKKRKMTTKQYCKRTTI |
| | LSHFHCFGSVE |
| 46 | ORF number 15 in reading frame 1 on the direct strand extends |
| | from base 22447 to base 22875 |
| | ctttggatgcctatgttaagagtgcagctgaacgtttctggcatccttctgcggtccctgaggc |
| | tttggtcagaaagaaggatccacttactggatcatggcaaggcccagacccagtcctcatatgg |
| | ggccgagggcatgtttgtgtttttccacaggatgcagaaggccctcggtggctgccagaacgat |
| | tggtgcgacatgtggaccctctacctgctgatgacattgatgaccctcagcaatgcagaagaag |
| | accagacgtattgggcctacgtacttgatccacctattctccaccctgctgtgtgggatggtcc |
| | tgagattccagactatgtcaatgacacTCACGCCCTAGGATTGCCTTCTGATGGACACATAAAA |
| | CATTTGGAAAGTTTTGTAAATCAGGCACTCCCTGCAGTCAGGTGA |
| 47 | Translation of ORF number 15 in reading frame 1 on the direct |
| | strand |
| | LWMPMLRVQLNVSGILLRSLRLWSERRIHLLDHGKAQTQSSYGAEGMFVFFHRMQKALGGCQND |
| | WCDMWTLYLLMTLMTLSNAEEDQTYWAYVLDPPILHPAVWDGPEIPDYVNDTHALGLPSDGHIK |
| | HLESFVNQALPAVR |
| 48 | ORF number 16 in reading frame 1 on the direct strand extends |
| | from base 23074 to base 23310 |
| | tacttaaacaaccatcttttgttatgcttcctgttaatatctctggaccttggtatactaaaag |
| | aaatttggcatgatgttaatgtgtctttagatatgtttcagcttcatgagaaaattcaaaatsc |
| | admtcatcagcaggtagagggtccacatgtcgcaccaatcgttctggcagccactgagggcctt |
| | ctgcatcctgtggaaaaacacaaacatgccctcggccccatatga |
| 49 | Translation of ORF number 16 in reading frame 1 on the direct |
| | strand |
| | YLNNHLLLCFLLISLDLGILKEIWHDVNVSLDMFQLHEKIQNXXHQQVEGPHVAPIVLAATEGL |
| | LHPVEKHKHALGPI |
| 50 | ORF number 17 in reading frame 1 on the direct strand extends |
| | from base 23362 to base 23859 |
| | ccaaagcctgaggggcagcagaaggatgccagaaacgttcagctgcactcttaccascadmctg |
| | gcattccttacaatccacagggacaagggattgttgaacgcactcatggcacattaaaagtcaa |
| | tttacaaaaaataaaaaagggggagtcatatcccctgacgccccataattatctgtctcattct |
| | ctctttattcaaaattttttgaccttggatgcccatggtaagagtgctgcagagcgcttttggc |
| | atccttccactgccactcaggctttggtcaaatggaaagacccacttacgggctcttggcaagg |
| | cccagatccagtcctcatatggggccgagggcatgtttgtgtttttccacaggatgcagaaggc |
| | cctcggtggctgccagaacgattggtgcgacatgtggaccctctacctgctgatgacattgatg |
| | accctaagcaatgcagaagaagaccagacgtattgggcctatgtacctga |
| 51 | Translation of ORF number 17 in reading frame 1 on the direct |
| | strand |
| | PKPEGQQKDARNVQLHSYXXXGIPYNPQGQGIVERTHGTLKVNLQKIKKGESYPLTPHNYLSHS |
| | LFIQNFLTLDAHGKSAAERFWHPSTATQALVKWKDPLTGSWQGPDPVLIWGRGHVCVFPQDAEG |
| | PRWLPERLVRHVDPLPADDIDDPKQCRRRPDVLGLCT |
| 52 | ORF number 18 in reading frame 1 on the direct strand extends |
| | from base 23947 to base 24384 |
| | tggacacatgaaacaacaTTTGGAAAGTTTTGTAAATCAGGCACTCCCTGCAGTCAGGTGACTG |
| | ATTTACAAGACGGGACTAGAGACTGGTCTAAGAAATCTGTTAATATATCTGCTTGTGTTCCTTC |
| | CCCATATACACTTTTGATTscadmttggtcaaatggaaagacccacttacaggctcttggcaag |
| | gcccagatccagtcctcatatggggccgagggcatgtttgtgtttttccacaggatgcagaagg |
| | ccctcggtggttgccagaacgattggtgcgacatgtggaccctctacctgctgatgacattgat |
| | gaccctcagcaatacagaagaagaccagacgtattgggcctatgtacctgatccaccaatcctc |
| | caccctgttgtatgggaaggtcctgagattccAGTscadmaaataaaactataa |
| 53 | Translation of ORF number 18 in reading frame 1 on the direct |
| | strand |
| | WTHETTFGKFCKSGTPCSQVTDLQDGTRDWSKKSVNISACVPSPYTLLIXXWSNGKTHLQALGK |
| | AQIQSSYGAEGMFVFFHRMQKALGGCQNDWCDMWTLYLLMTLMTLSNTEEDQTYWAYVPDPPIL |
| | HPVVWEGPEIPVXXIKL |
| 54 | ORF number 19 in reading frame 1 on the direct strand extends |
| | from base 24625 to base 24948 |
| | cgccccataattacttgtctttttattcaaaattttttgactttggatgcctatgttaagagtg |
| | cagctgaacgtttctggcatccttctgccgaccctgaggctttggtcagaaagaaggatccact |
| | tactggatcatggcaaggcccagacccagtcctcatatggggccgagggcatgtttgtgttttt |
| | ccacaggatgcagatagtcctcggtggctgccagaacgattggtgcgacatgtggaccctctac |
| | ctgctgatgacattgatgaccctcagcaatgcagaagaagaccagacgtattgggcctacgtac |
| | ctga |
| 55 | Translation of ORF number 19 in reading frame 1 on the direct |
| | strand |
| | RPIITCLFIQNFLTLDAYVKSAAERFWHPSADPEALVRKKDPLTGSWQGPDPVLIWGRGHVCVF |
| | PQDADSPRWLPERLVRHVDPLPADDIDDPQQCRRRPDVLGLRT |
| 56 | ORF number 20 in reading frame 1 on the direct strand extends |
| | from base 25126 to base 25380 |
| | ACCACTGTTGTTAAAACTGTTAATATATCtgcttgtgttccttccccttatatacttttgatta |
| | aaaatattaatgtacacscadmagaacaggtctggggtattttccccaggggtcatagatttac |
| | ctgtactccaccaaaaaactacaaaggcaataatttggaaaacagatacacctgtgtggataga |
| | tcagtggccccttacacaggaaaagatatcggccgcccaggcgcttgtacaggagcagcttga |
| 57 | Translation of ORF number 20 in reading frame 1 on the direct |
| | strand |
| | TTVVKTVNISACVPSPYILLIKNINVHXXEQVWGIFPRGHRFTCTPPKNYKGNNLENRYTCVDR |
| | SVAPYTGKDIGRPGACTGAA |
| 58 | ORF number 21 in reading frame 1 on the direct strand extends |
| | from base 28306 to base 28737 |
| | ccttggatgcccatggtaagagtgctgcagagcgcttttggcatccttctactgccactcaggc |
| | tttggtcaaatggaaagacccacttacaggctcttggcaaggcccagatccagtcctcatatgg |
| | ggccgagggcatgtttgtgtttttccacaggatgcagagggccctcggtggctgccaagacgat |
| | tggtgcgacatgtggaccctctacctgctgatgacattgatgaccctcagcaatgcagaagaag |
| | accagacgtattgggcctatgttcctgatccaccaatcctccaccctgctgtatgggaaggtcc |
| | tgagattccagactatgtcaatgacactcacgccctaggattgccTTCTGATGGACACATAAAA |
| | CAACATTTGGAAAGTTTTGTAAATCAGGCACTCCCTGCAGTCAGGTGA |
| 59 | Translation of ORF number 21 in reading frame 1 on the direct |
| | strand |
| | PWMPMVRVLOSAFGILLLPLRLWSNGKTHLQALGKAQIQSSYGAEGMFVFFHRMQRALGGCQDD |
| | WCDMWTLYLLMTLMTLSNAEEDQTYWAYVPDPPILHPAVWEGPEIPDYVNDTHALGLPSDGHIK |
| | QHLESFVNQALPAVR |
| 60 | ORF number 22 in reading frame 1 on the direct strand extends |
| | from base 30907 to base 31191 |
| | ctttggatgcccatggtaaaagtgcagctgcacgttttttggcatccttcaactagccctcagg |
| | ccttggtcaaatggaaggacccacttacgggtgtctggcaaggcccagatccagtcctcatatg |
| | gggccgagggcatgtttgtgtttttccacaggatgcagatagtcctcggtggctgctagaacga |
| | ttggtgcgacatgtggaccctctacctgctgatgacattgatgaccctcagcaatgcagaagaa |
| | gaccagacgtattgggcctacgtacctga |
| 61 | Translation of ORF number 22 in reading frame 1 on the direct |
| | strand |
| | LWMPMVKVQLHVFWHPSTSPQALVKWKDPLTGVWQGPDPVLIWGRGHVCVFPQDADSPRWLLER |
| | LVRHVDPLPADDIDDPQQCRRRPDVLGLRT |
| 62 | ORF number 23 in reading frame 1 on the direct strand extends |
| | from base 31279 to base 32070 |
| | TGGACACATGAAACAACATTTGGAAAGTTTTGTAAATCAGGCACTCCCTGCAGTCAGGTGACTG |
| | ACTTACAAGACGGGACTAGAGACTGGTCTAAGAAATCTGTTAATGTATCTGCTTGTGTTCCTTC |
| | CCCTTATACACTTTTGATTGAAAATATTAATGTACATTTTGTAGGAGTTCAGTTTAtggaagat |
| | gtgattcagagtataaaagttaaatcttatttagaatgtcattcagaatatcattggatacgtg |
| | ttacttctaaaaggtataataatagtcaatatgattggaatcgggttcgtttacatcttcaagg |
| | aatttggcatgatgctaatgtgtctttagatascadmCGAGGAGTGCAGATAGAGCCGGCGGCG |
| | GCGGCGCAGCGAGCGAGCAGTGACCGCGCTCCTACCCAGTTCTGCCCCACGGCTCCTACCTGCT |
| | TGCCTCCCTCAGCCCCTCGCCCGGCTGTGACTAACCGCGACCATGATGTTCTCCAGCTTCAACG |
| | CCGACTACGACGCGGCCTCTTCCCGCTGCAGCAGCGCCTCCCCAGCTGGGGACAGTCTCTCCTA |
| | CTACCACTCACCCGCCGACTCCTTCTCCAGCATGGGCTCTCCTGTCAATGCGCAGGTAAGGCTG |
| | GCTTCACCGAGCCCAGGGCTCGGGGTCACTGGGGTGGAGGCATCGGGCGGGAAGCTCAGGAAGA |
| | CGAGTCGGGTACCCCTTTTGGCGGGGAGGGAGCAGCCCTAACTCGCGAGTCCCGGACTTGTGGG |
| | GCGCTCACACACGCTTGTCAGTAA |
| 63 | Translation of ORF number 23 in reading frame 1 on the direct |
| | strand |
| | WTHETTFGKFCKSGTPCSQVTDLQDGTRDWSKKSVNVSACVPSPYTLLIENINVHFVGVQFMED |
| | VIQSIKVKSYLECHSEYHWIRVTSKRYNNSQYDWNRVRLHLQGIWHDANVSLDXXRGVQIEPAA |
| | AAQRASSDRAPTQFCPTAPTCLPPSAPRPAVTNRDHDVLQLQRRLRRGLFPLQQRLPSWGQSLL |
| | LPLTRRLLLQHGLSCQCAGKAGFTEPRARGHWGGGIGREAQEDESGTPFGGEGAALTRESRTCG |
| | ALTHACQ |
| 64 | ORF number 24 in reading frame 1 on the direct strand extends |
| | from base 34747 to base 35073 |
| | CAGACCTCCTGCCCTGGCGGATGCCATGGATTCCAGAGCCCTAGTCTCCCACCCCTCACTGTCG |
| | CAGGACAGTCTGGGCATGTTTGCACATGCTCCTGCTGCACAGGGCACTCTCTCGTAATGTATCT |
| | CAGAGTTCAGTCCCATAGATGGCCTTATAACGTAAGTACTCTTCTAAGCACTGAAGGACATTAT |
| | CATCCACTTTGGGGTCAAACTTGTTGGCCAACAGGTGAGGGTTACGAAGAATCCAGTGCAGGTC |
| | CCCAGCCCCATAAATGCAGATACCCCGCTGGTGGGTTCCAGAGCAAGGTCCATAAGGTGCCCCC |
| | TTACTGA |
| 65 | Translation of ORF number 24 in reading frame 1 on the direct |
| | strand |
| | QTSCPGGCHGFQSPSLPPLTVAGQSGHVCTCSCCTGHSLVMYLRVQSHRWPYNVSTLLSTEGHY |
| | HPLWGQTCWPTGEGYEESSAGPQPHKCRYPAGGFQSKVHKVPPY |
| 66 | ORF number 25 in reading frame 1 on the direct strand extends |
| | from base 36097 to base 36516 |
| | GAGAAAGTCTCAGAGCGACAATGGCCAGCAGGAAATAGCAGCCCAGAGCCCACAGGTAGTGCTT |
| | CTGGAAGAGTTTCTTCTTCCACCAAATCATCTTCATGGAATGGAAGATCGGTAGAATTTGGGCA |
| | CCAGGAAGAAGAAGGATGGGATCCTTscadmACCCTGGCCGCGGGGGCGGCGCGCACCGTCCAC |
| | GCGTCCGGGGCCCAGCGGGGCCGGGCCCGGAGTCGGCATGAATCGCTGCTGGGCGCTCTTCCTG |
| | TCTCTCTGCTGCTACCTGCGTCTGGTCAGCGCCGAGGTGAGTTGCGACAGCCGTGGGGCTGGTT |
| | CGCTTCATTCATTGCCCCCACCCCCATCCCTGTTGCCCCCTCCCCTCCCTGCAGTGAACTTTGG |
| | ACCCTTGCAGCCCGTGGGCCTGGCGCCCGGCGCTAG |
| 67 | Translation of ORF number 25 in reading frame 1 on the direct |
| | strand |
| | EKVSERQWPAGNSSPEPTGSASGRVSSSTKSSSWNGRSVEFGHQEEEGWDPXXTLAAGAARTVH |
| | ASGAQRGRARSRHESLLGALPVSLLLPASGQRRGELRQPWGWFASFIAPTPIPVAPSPPCSELW |
| | TLAARGPGARR |
| 68 | ORF number 26 in reading frame 1 on the direct strand extends |
| | from base 36649 to base 36957 |
| | TCTTATCCCCCACCTCCTCAGAAACCCCAGAATAAGCCCCTAACTGGCCTAAGGGAGAGGGGGT |
| | GGGGTGGTGCCGAGGGTGCAGAAGGCGGCGCGTCCTTCCAAGCCCACTTCAGTTCCAGCTTAGG |
| | TTCTGTCCGGGAACCGGCTTGCACGGAAGGTGCGAGCTCGCGCACTGGTGGCAGCCACGCCAAC |
| | CTACGGCAGGGGTTTGCGTCCCACCCTGGCTCCCGCTCCAGCTCTTGCTTGCTCGGCCCCAGAG |
| | CGTGGTGCAGGAGCAGCTTGTGTCTTGGGCGCGGCGGGGGTACAGAGAGATAG |
| 69 | Translation of ORF number 26 in reading frame 1 on the direct |
| | strand |
| | SYPPPPQKPQNKPLTGLRERGWGGAEGAEGGASFQAHFSSSLGSVREPACTEGASSRTGGSHAN |
| | LRQGFASHPGSRSSSCLLGPRAWCRSSLCLGRGGGTER |
| 70 | ORF number 27 in reading frame 1 on the direct strand extends |
| | from base 37270 to base 38031 |
| | GGTGAAGAGGCTCAGGGGCTGCGAGGAGCGCTACGCGCCTGGTCCCGTCCCGCCTCAGCTCGGC |
| | GGCCGCCGGGAGCCCGCACCGAGCCGGCTCCTGGGAGGGCCGGCCCCTCTCGGGCCTCCAACGA |
| | GGAGCAGGAAGGAGGCGGCGGCGGCGGCGAAGGGGTTAAGGTGAAGGGCTTCGAGGCCGCGGCC |
| | GGGCCTTGGGCCGCAGCCAGCGCAGGTTGTTTTGACCACGGAGGAGCCGTCTCCGTCTCCTTTT |
| | GTTCTCGGGGCTCCTCGAGGGCCGCCGGCCGTCCGCCCTGGGGCCCCGCCCTTCCGCGGCCGTC |
| | CCCCGTGGCCCGCACCCGGGAGGGAGGACGCGGGGATCAGCCTGGCTGCCTGCAGTCCCCTCCC |
| | GACGCCCCCTCCTCTCCTCCTGCTGATGCCCCCCGGGCCGCGGCCAGCTGTTGGGGCGGGGGGC |
| | GCCGGCCGGCCCCAGCTGCCGCCTCGCCGCggggcctgggggctgggccctgtgccagggcgtc |
| | ctgggAACGGCGGCGCCCCAGCCGCTGCTCTCCGCAGCCCACCCCGCCCGGCCCCCCGACTCGC |
| | TCACTCACCCCACGCATGCACACTCTTGGCCGGAGGCGATGCTGCGCTCCGGCGGGCGGGCGCG |
| | CAGGGCGACGGGCACGCACTGGCGCGGCCGGGTcgcgcgcccgccgccacgcccgtgcacatgc |
| | gggacacacgcgcgcgcactacacacacacacgcatggtccccgcacacacggcttga |
| 71 | Translation of ORF number 27 in reading frame 1 on the direct |
| | strand |
| | GEEAQGLRGALRAWSRPASARRPPGARTEPAPGRAGPSRASNEEQEGGGGGGEGVKVKGFEAAA |
| | GPWAAASAGCFDHGGAVSVSFCSRGSSRAAGRPPWGPALPRPSPVARTREGGRGDQPGCLQSPP |
| | DAPSSPPADAPRAAASCWGGGRRPAPAAASPRGLGAGPCARASWERRRPSRCSPQPTPPGPPTR |
| | SLTPRMHTLGRRRCCAPAGGRAGRRARTGAAGSRARRHARAHAGHTRAHYTHTRMVPAHTA |
| 72 | ORF number 28 in reading frame 1 on the direct strand extends |
| | from base 38401 to base 38718 |
| | GCTTCCGAGGGGGCTCCCACCCCCCACTGTTCTGTGCTCTTTGCTGATCCCAGCCAGCACGCTG |
| | CAGAGAGGCTGGGTGACAGCTGGATAAGGCTTTCCCGCCTGCCCTTACCATTCCCAGCTTCATC |
| | CAGCACCTCCTCCTCCTTTCCCACAACTCCCTGGGTGTGTGTTTGGGGGGTGAGCCTATGGCAC |
| | AGAAACTGGTGCCTGTCTCCTCACTTTAATCACAGCATCCTTGGACACATGGCTCTCAGGAACC |
| | CACAGTTGTGTGGTGCTTTGCAGTTTACGAAGCACTTTCCTGCTAAGCCTTACTCTGAGTAA |
| 73 | Translation of ORF number 28 in reading frame 1 on the direct |
| | strand |
| | ASEGAPTPHCSVLFADPSQHAAERLGDSWIRLSRLPLPFPASSSTSSSFPTTPWVCVWGVSLWH |
| | RNWCLSPHENHSILGHMALRNPQLCGALQFTKHFPAKPYSE |
| 74 | ORF number 29 in reading frame 1 on the direct strand extends |
| | from base 39607 to base 39849 |
| | TCCTCAGCCCAGGGTAGCCTAGAATGGCCACACTGCTCTTCACCAGGCATCCTCATTCGAGCCC |
| | CCCCGGCCCCCCATCTTGAGAGACAAGCATATCTTTCTTTTCCATGTCTTGGGCTGCCAATATT |
| | GGACAGGACAGAGGGGAAGAAACAGAAGGAAAATCAGATCGCAAGGCTTCTGTGTATCTTGAGC |
| | AGGCCTGGGCCTCAGTTGCCGCCGCGTGAGAATATGAGAAGGTTGGATTAG |
| 75 | Translation of ORF number 29 in reading frame 1 on the direct |
| | strand |
| | SSAQGSLEWPHCSSPGILIRAPPAPHLERQAYLSFPCLGLPILDRTEGKKQKENQIARLLCILS |
| | RPGPQLPPRENMRRLD |
| 76 | ORF number 30 in reading frame 1 on the direct strand extends |
| | from base 41215 to base 41634 |
| | gCAATCCAGGTTTCCTTGGCAGCTGAAGCTCTACAgtttctctgcctctccactgttgacattt |
| | ggggccagacagttcttgattgtgggggaggctgtcctgtgcatagtaggatgtttagcagcaa |
| | ccctggcctctacctactagacaccagtagcaggcctccagttgtgataaccaaaagtgcctcc |
| | agacctggccagtgtcccctgggggtcaCTCACTCCCTGCTCTATGACCTCCACTGGGTGAAGA |
| | GTGGACCTGAACTGAAAACAGTCCATGAAAGAGGGAGGGGCCCGCTCTGCTCCTTACCAGTCGT |
| | GTTGACCTTTAGCCATTTACTTAATTTTTCTAAGCCTCAGCTTCCTCATTTGGAAGACAGGGAT |
| | ACAAACAGTGACAGCCTCTTGATTGTATTTGATTGA |
| 77 | Translation of ORF number 30 in reading frame 1 on the direct |
| | strand |
| | AIQVSLAAEALQFLCLSTVDIWGQTVLDCGGGCPVHSRMFSSNPGLYLLDTSSRPPVVITKSAS |
| | RPGQCPLGVTHSLLYDLHWVKSGPELKTVHERGRGPLCSLPVVLTFSHLLNFSKPQLPHLEDRD |
| | TNSDSLLIVFD |
| 78 | ORF number 31 in reading frame 1 on the direct strand extends |
| | from base 41872 to base 42114 |
| | GGGAATCATGCAGGAGAGAACCCCAGGGAGAAGGGGAGAGTCCTTCATGCATTTTACCAGTGTT |
| | TAGTGAGCACCTACTCTGTGCTTTCCCCCAGTCTCTGTCCTGGGCTCTTCCCCGTGCAGGCTGG |
| | GAGGGTGGGGTTCTGGGTTTGTTTCCATAAGACATCATCGTCTCTTTTTTATTATAGGCCGGGT |
| | CCAGGGTGTCCACTGGGCCCAGCTGGGATCTGCCTACTCTGCCATGGCTAG |
| 79 | Translation of ORF number 31 in reading frame 1 on the direct |
| | strand |
| | GNHAGENPREKGRVLHAFYQCLVSTYSVLSPSLCPGLFPVQAGRVGFWVCFHKTSSSLFYYRPG |
| | PGCPLGPAGICLLCHG |
| 80 | ORF number 32 in reading frame 1 on the direct strand extends |
| | from base 42115 to base 42393 |
| | CAGCTGCAGCCAGCTCTCCAGTGGGCAAGGAGGTCTTGGCATGAGTGTTACGTGCCATTTGGTA |
| | CTGGGTCTTCAGTCCGCTCTCCTAAGAGGTTAATTGATTCATTATGCCACAAACAGCCTGGGAG |
| | ACCTGGCTGGGCACCCCCACTTCGGCTTCCTCTGCTGCTGCCTCTCCTGCCAACCCCAGACAGA |
| | ATTAGAATTAAAATCAAATCAAATGGCTACAACCCCCTCAGTTCACAGGTGATAGCCAGGACCC |
| | GAGAGGGGCAGCAACCAACCTGA |
| 81 | Translation of ORF number 32 in reading frame 1 on the direct |
| | strand |
| | QLQPALQWARRSWHECYVPFGTGSSVRSPKRLIDSLCHKQPGRPGWAPPLRLPLLLPLLPTPDR |
| | IRIKIKSNGYNPLSSQVIARTREGQQPT |
| 82 | ORF number 33 in reading frame 1 on the direct strand extends |
| | from base 44644 to base 44922 |
| | AGGGTGGGGCTGTGGGAGGGGAGGCAGGCAGGGAGAAGGTGCCCAGGGCATCTGCACCCTGAGT |
| | ATCCAGGTGTGGACTCAGCCAGGGAGGGTGGTGCTGGAGGAGCCACCTCCCTGTCTCTCTGGCC |
| | AAAGGCCCGCTCTACAAGGTCTCCCGGGGACACCTGGCCGGGACCAGTGGGCAGCCCTGCCCGT |
| | GCCCAAGAGGGCACTCAGAGAATGGGCACGTGCTTGGTGGCACACACGTGGCAGGGCTGGCGGG |
| | CTGTGTCGGGAATGTATTTATAA |
| 83 | Translation of ORF number 33 in reading frame 1 on the direct |
| | strand |
| | RVGLWEGRQAGRRCPGHLHPEYPGVDSAREGGAGGATSLSLWPKARSTRSPGDTWPGPVGSPAR |
| | AQEGTQRMGTCLVAHTWQGWRAVSGMYL |
| 84 | ORF number 34 in reading frame 1 on the direct strand extends |
| | from base 44923 to base 45165 |
| | ACGCTGTCTTCAGAGCAAATTCCATTCTATTCTAACCTCTGGCCTGTTCCCTGGAGCCCTGGTC |
| | AGCACCCCCCTGCACCCCCAGCTCCCCTTCCCTCTGGGGTTTTGTCTCTTTGTCACTTTGTAAT |
| | CCTTGCCCAGACTGCTATCTACGGGGGACAGCATTTCCTGCCTTTGTTTCCTCTCCCAGTTGGG |
| | CCCCTGGCTCCCTCTCAAAAGCATTCCCCGGGCCCTTTCAAACCCGCCTAG T |
| 85 | Translation of ORF number 34 in reading frame 1 on the direct |
| | strand |
| | TLSSEQIPFYSNLWPVPWSPGQHPPAPPAPLPSGVLSLCHFVILAQTAIYGGQHFLPLFPLPVG |
| | PLAPSQKHSPGPFKPA |
| 86 | ORF number 35 in reading frame 1 on the direct strand extends |
| | from base 45313 to base 45786 |
| | CTTTTCTGCTGTTTCTTTCCAAGGTCCTTCGCCCCCACCCTCATATTGCCCCTCCACACCCCGG |
| | GTGGGGGTCGGGTCGGAGAAGACGAGGTTTTCAATAGCAGGCCTGTTTCGAGGCAACCATGTGG |
| | CTATTTTTTCCTAATCAACTTAACCTTTCCACAAAGCACATCTTTTCCCCATCTCCTCCCAACC |
| | AGGGACATTCCAGAAATGGCAGAGAGAAAGGAATGGAGCCAGAGGGACAGACAGACACACTGTT |
| | CGTGGGACAATAGGCTAGACGGAAGTGCATCAGTTTTAGGAAAGTCTGCTCTAAACAGGGCCCC |
| | TTGGGAGCCCACAGGGACGAGCAATAGTTTTGTCATGGGCAGTGGCAGTGGGATGGGGAGACAG |
| | TGTGACCCTGAGATGCTGTGTGGAGGGGGACAGAGCTTGTCCCCGACACCCTTCAGTGTATTTG |
| | CTGGCTTTCAGCCATCAGAGAGCTAG |
| 87 | Translation of ORF number 35 in reading frame 1 on the direct |
| | strand |
| | LFCCFFPRSFAPTLILPLHTPGGGRVGEDEVFNSRPVSRQPCGYFFLINLTFPQSTSFPHLLPT |
| | RDIPEMAERKEWSQRDRQTHCSWDNRLDGSASVLGKSALNRAPWEPTGTSNSFVMGSGSGMGRQ |
| | CDPEMLCGGGQSLSPTPFSVFAGFQPSES |
| 88 | ORF number 36 in reading frame 1 on the direct strand extends |
| | from base 45787 to base 46023 |
| | AAGAGTCTGCCCACCATTCAACGTCAAGCTCAAAGTTCCCCTGTCCAGCCCTCACTTTCCGCAG |
| | CCGGCTTCCGGCTGCCTCTACCCAGAGGGATGTCTCCAAGGAGTGCTGATGGTGCTGAGATGAG |
| | GGCCTCCAGGCTAGAGAAGGGAGCTGTAGTTGTGACCTTAGGAATAAATGTACAGCTTAGGGCA |
| | GGCATGGGGCAAAAGGTCAGAGGGAGAGAGACAGAAACACAATGA |
| 89 | Translation of ORF number 36 in reading frame 1 on the direct |
| | strand |
| | KSLPTIQRQAQSSPVQPSLSAAGFRLPLPRGMSPRSADGAEMRASRLEKGAVVVTLGINVQLRA |
| | GMGQKVRGRETETQ |
| 90 | ORF number 37 in reading frame 1 on the direct strand extends |
| | from base 46072 to base 46383 |
| | GGGCTCCCCTATCCCAGCAGTTCCAGctccctacctctctctgcctttagtccccaccccaccc |
| | caccccacccctctccttcccaccctctctcccgcccaacTGAACCATTGTCAGGGGCTCCACA |
| | GGGGCTGTGTCCAGGGCATGCTGGTCCCCCCTGGGGACTATGGGAATTTCTCCATTCAGCACTT |
| | CCTATGGGAACGCTGGGTGGAGGGGCACTGGAAAGTGGCCTCAGAGCTCTGGGTCCTTGCCCTG |
| | CCCTGGAGGCCGAGGAGGGTTCGCTTACAGTAGCAAAAGGGAACGGTTATTTTTAA |
| 91 | Translation of ORF number 37 in reading frame 1 on the direct |
| | strand |
| | GLPYPSSSSSLPLSAFSPHPTPPHPSPSHPLSRPTEPLSGAPQGLCPGHAGPPWGLWEFLHSAL |
| | PMGTLGGGALESGLRALGPCPALEAEEGSLTVAKGNGYF |
| 92 | ORF number 38 in reading frame 1 on the direct strand extends |
| | from base 46576 to base 46890 |
| | GGGGCAAGAGGCAATCCTTCCGTCTGTCCCAGAGCCCCCACTGGAGTCCCCAGCCCGTGGTATG |
| | ACCAGCCAGCACTTGTCACAGTGCTTCTGACTGTGCCTTCTCTTGCAGATGAAGACGGGGCTGA |
| | GTTGGACCTGAATTTGACTCAGTCCCATTCTGGAGGCAAGCTGGAGAGCTTATCCCGAGGGAGA |
| | AGGAGCCTAGGTAAGAATGAGGGTGCAAACGGGGGCCCCTCAAAGGTGGGGGCCAGGGAAGAAG |
| | AACTGAGCACACAGCCTGCCGGAGGCTGTGAGGGTGGGCCCTGTTTGTCCCACACTTAG |
| 93 | Translation of ORF number 38 in reading frame 1 on the direct |
| | strand |
| | GARGNPSVCPRAPTGVPSPWYDQPALVTVLLTVPSLADEDGAELDLNLTQSHSGGKLESLSRGR |
| | RSLGKNEGANGGPSKVGAREEELSTQPAGGCEGGPCLSHT |
| 94 | ORF number 39 in reading frame 1 on the direct strand extends |
| | from base 47176 to base 47406 |
| | GGGCTGTGGCCTGGGACAGCAGGCATGGAGCAAGCCTGGGACCTGCCTCCTGCTGTACTGCAGA |
| | AACCAAAAGGAGAATGTAGATCAGGGAAGGCAAGTGCCCACTCCACGCCCCTCTTCCTCTGTGC |
| | CCACCTGCAGTCCCCAAAACACTGTAGACAGTGGCTGGGGGGCCTCCAGGTAAGAGTCAGTGGC |
| | CTGAGTTCCACTCTTTGCTCTGTGAATTTGGGCATCTAA |
| 95 | Translation of ORF number 39 in reading frame 1 on the direct |
| | strand |
| | GLWPGTAGMEQAWDLPPAVLQKPKGECRSGKASAHSTPLFLCAHLQSPKHCRQWLGGLQVRVSG |
| | LSSTLCSVNLGI |
| 96 | ORF number 40 in reading frame 1 on the direct strand extends |
| | from base 47863 to base 48297 |
| | CAGCCAGTACTTAAAACCTCCCCTGACGTGGAAGGAAGAGGGAATCGGCCAACCGTTTTGGAGG |
| | TGCTCCACTTCCTGCCGGCTAGGGGCCCTGAGCAGCCCTCCACCCCACTCCTGACGGAGTTCCC |
| | TCTCCCTTCAGATTCCCAGCCGGTCGCCGAGCCAGCCATGATCGCGGAGTGTAAAACGCGCACT |
| | GAGGTGTTTGAGATCTCCCGGCGCCTGGTTGACCGCACCAACGCCAACTTCCTGGTGTGGCCGC |
| | CCTGCGTGGAGGTGCAGCGCTGCTCCGGCTGCTGCAACAATCGCAACGTGCAGTGCCGCCCCAC |
| | CCAGGTGCAGCTGCGACATGTCCAGGTGTGCAGGCCCCACGTCCCCTCCTGGGCTGGCCCAGCT |
| | GAGAGCAGGGGCTGCCCCTCTGGGGCTGGCACTCACGGACCAGGCTCTTGA |
| 97 | Translation of ORF number 40 in reading frame 1 on the direct |
| | strand |
| | QPVLKTSPDVEGRGNRPTVLEVLHFLPARGPEQPSTPLLTEFPLPSDSQPVAEPAMIAECKTRT |
| | EVFEISRRLVDRTNANFLVWPPCVEVQRCSGCCNNRNVQCRPTQVQLRHVQVCRPHVPSWAGPA |
| | ESRGCPSGAGTHGPGS |
| 98 | ORF number 41 in reading frame 1 on the direct strand extends |
| | from base 48298 to base 48570 |
| | ATGCGTCAAAAGGCATTCCTGGCAGGGTGTGGGCTCAGTCCAGAGAAGGCGCTCTCAGGAAGCT |
| | CTCCGGACAGGTGTGCGGAGGCTGCCCAAGAATCCTCTATGGCCTCCCAAGCCACTGTGACAAA |
| | AAGTCACAGGCAGACCTCCAGACAGGCTGGGTATGGGACATTAAGTAAAAGGCATTGCCTCATT |
| | CTTTACAGGGATAAAATCCCAAAATGTCTCTTGAAGAGACATGTCTACAAACATATTGGACCCT |
| | CAGGATGTTCTGGGTAG |
| 99 | Translation of ORF number 41 in reading frame 1 on the direct |
| | strand |
| | MRQKAFLAGCGLSPEKALSGSSPDRCAEAAQESSMASQATVTKSHRQTSRQAGYGTLSKRHCLI |
| | LYRDKIPKCLLKRHVYKHIGPSGCSG |
| 100 | ORF number 42 in reading frame 1 on the direct strand extends |
| | from base 49246 to base 49800 |
| | AGCCTCCCCTCACTTCCTTCCAGACAACCATCTCCCGCCTCTGCCACAGCCCCTGACCTTGGCT |
| | GGCGCTCCAGGAATGAGGACACCACAGGCTCCACGCTCCACCCGGAAATGCCTTTCTCCCTCTC |
| | TGAGAGCACCGAGGGGGGCTGTGGCCAAGCTGGAGGCCAGGTCGGGAGGGCTTGTTTTGATGGA |
| | AAAGCTACAAGAAGGGCAGAGGGCAAGGTCCTGCTATTGTTTTGGCCGCAGTGTCTGCACTGCT |
| | GCTCTTCAGGCTTTCGAGGAAAGATTCCCCACAGAGGACGCTGGGGTGGGAAGAGAAGGCAGGC |
| | AGCTACCTCAGCCCCTGCCCAAGTGGTCTTACAGAGGCACTTGGGTGGTTCTGTCCTCCAGGTG |
| | AGGAAGATCGAGATTGTACGGAAGAAGCCAAGCTTTAAGAAGGCCACAGTGACCCTGGAGGACC |
| | ACCTGGCGTGCAAGTGTGAGACGGTAGTGGCTGCACGACCTGTGACCCGAAGCCCAGGGAGTTC |
| | CCAGGAGCAGCGAGGTAACCTTCAGTCCAGGGTTGGTCTCTGA |
| 101 | Translation of ORF number 42 in reading frame 1 on the direct |
| | strand |
| | SLPSLPSRQPSPASATAPDLGWRSRNEDTTGSTLHPEMPFSLSESTEGGCGQAGGQVGRACFDG |
| | KATRRAEGKVLLLFWPQCLHCCSSGFRGKIPHRGRWGGKRRQAATSAPAQVVLQRHLGGSVLQV |
| | RKIEIVRKKPSFKKATVTLEDHLACKCETVVAARPVTRSPGSSQEQRGNLQSRVGL |
| 102 | ORF number 43 in reading frame 1 on the direct strand extends |
| | from base 53419 to base 53697 |
| | TATTGTTCCCCTCGTCCGTCTGTCTCGATGCCTGATTCGGACGGCCAATGGTGCTTCCCCGCCC |
| | CTCCACGCGTCCGTCCACCCCTCTGCCAGTGGGTCTCCCCTCAGTGGCscadmCTCAGGGGCTG |
| | CGAGGAGCGCTACGCGCCTGGTCCCGTCCCGCCTCAGCTCGGCGGCCGCCGGGAGCCCGCACCG |
| | AGCCGGCTCCTGGGAGGGCCGGCCCCTCTCGGGCCTCCAACGAGGAGCAGGAAGGAGGCGGCGG |
| | CGGCGGCGAAGGGGTTAAGGTGA |
| 103 | Translation of ORF number 43 in reading frame 1 on the direct |
| | strand |
| | YCSPRPSVSMPDSDGQWCFPAPPRVRPPLCQWVSPQWXXLRGCEERYAPGPVPPQLGGRREPAP |
| | SRLLGGPAPLGPPTRSRKEAAAAAKGLR |
| 104 | ORF number 44 in reading frame 1 on the direct strand extends |
| | from base 53698 to base 54324 |
| | AGGGCTTCGAGGCCGCGGCCGGGCCTTGGGCCGCAGCCAGCGCAGGTTGTTTTGACCACGGAGG |
| | AGCCGTCTCCGTCTCCTTTTGTTCTCGGGGCTCCTCGAGGGCCGCCGGCCGTCCGCCCTGGGGC |
| | CCCGCCCTTCCGCGGCCGTCCCCCGTGGCCCGCACCCGGGAGGGAGGACGCGGGGATCAGCCTG |
| | GCTGCCTGCAGTCCCCTCCCGACGCCCCCTCCTCTCCTCCTGCTGATGCCCCCCGGGCCGCGGC |
| | CAGCTGTTGGGGCGGGGGGCGCCGGCCGGCCCCAGCTGCCGCCTCGCCGCggggcctgggggct |
| | gggccctgtgccagggcgtcctgggAACGGCGGCGCCCCAGCCGCTGCTCTCCGCAGCCCACCC |
| | CGCCCGGCCCCCCGACTCGCTCACTCACCCCACGCATGCACACTCTTGGCCGGAGGCGATGCTG |
| | CGCTCCGGCGGGCGGGCGCGCAGGGCGACGGGCACGCACTGGCGCGGCCGGGTcgcgcgcccgc |
| | cgccacgcccgtgcacatgcgggacacacgcgcgcgcactacacacacacacgcatggtccccg |
| | cacacacggcttgagcacacgtgcgcgcacacccacgcacgcacAGCCTAG |
| 105 | Translation of ORF number 44 in reading frame 1 on the direct |
| | strand |
| | RASRPRPGLGPQPAQVVLTTEEPSPSPFVLGAPRGPPAVRPGAPPFRGRPPWPAPGREDAGISL |
| | AACSPLPTPPPLLLLMPPGPRPAVGAGGAGRPQLPPRRGAWGLGPVPGRPGNGGAPAAALRSPP |
| | RPAPRLAHSPHACTLLAGGDAALRRAGAQGDGHALARPGRAPAATPVHMRDTRARTTHTHAWSP |
| | HTRLEHTCAHTHARTA |
| 106 | ORF number 45 in reading frame 1 on the direct strand extends |
| | from base 54394 to base 54621 |
| | CTCTGTTTCCTTCTTGGGTGTTCTGAGGGAGGGGAAACAGGAACCCTCCCTCGGGTCCCTCTCC |
| | ACAGCACCCATGGGTGTGTTTTTTTTTTTTTTTGGTCAGGTCAGTTCCACACCCTTTGCGCATT |
| | ACCCTTCTATGATTGCTTTCTTTCAGCCACTCCCATGTGGCTGAAAATAGTGTCGATGTGCTTG |
| | GGGGGTACTGTTCAGAGCATTTCTCCCTTCAAGTAA |
| 107 | Translation of ORF number 45 in reading frame 1 on the direct |
| | strand |
| | LCFLLGCSEGGETGTLPRVPLHSTHGCVFFFFWSGQFHTLCALPFYDCFLSATPMWLKIVSMCL |
| | GGTVQSISPFK |
| 108 | ORF number 46 in reading frame 1 on the direct strand extends |
| | from base 54838 to base 55116 |
| | GCCTATGGCACAGAAACTGGTGCCTGTCTCCTCACTTTAATCACAGCATCCTTGGACACATGGC |
| | TCTCAGGAACCCACAGTTGTGTGGTGCTTTGCAGTTTACGAAGCACTTTCCTGCTAAGCCTTAC |
| | TCTGAGTAAGCAAGCCTCAGGCAGCTCTTGGGGAAGAGACCTAAAGGGAAAACCTATCGACATG |
| | GGGACCAGTCCAGGAAGGTGGACTTCAGGAGATCTTACTGGCAGAGGTGGCCTTGGGGCTGGCC |
| | ACGTCTCAGGCCTGTGTGGCTGA |
| 109 | Translation of ORF number 46 in reading frame 1 on the direct |
| | strand |
| | AYGTETGACLLTLITASLDTWLSGTHSCVVLCSLRSTFLLSLTLSKQASGSSWGRDLKGKPIDM |
| | GTSPGRWTSGDLTGRGGLGAGHVSGLCG |
| 110 | ORF number 47 in reading frame 1 on the direct strand extends |
| | from base 56464 to base 56892 |
| | ATTAGGCCTGAGACTCTCAGGCAGGCTGGCGCTTGGAGGTATGGTCGGCTTCTGCCTCTGCCAA |
| | CCATAGACAACGCCCCTGGGTGCTGGGGCCAAGAGCGACGTCCTCTCTCAGCTGAACGGCGCAC |
| | TGGGGAgtgtgtatctgtgtgcagagtgtgtgtctgtgtgCGCTGGGGCCCAGGTGGAGGGTGG |
| | GGTCCAAGCCCCTTTGATCTGCCAGCATGGTTGGGAGCAGGTAATTCACCTGGCCTCACGCTTC |
| | CTACCTTCTGCAGCTGGTGTTGGGGGTGGGGTGGGGTGGGGAAGAGACTGTTTGCCTTGGCTCC |
| | CAAGGCTGGCTGTGCCCCAGCTGCCTTCTCGCCACGCCCTCACCCTGCTAGGAACCCCAGGCCT |
| | GAGATCTGGGACAGTTTCCTCATAGTACCAAGCCTCCTTTCCTAG |
| 111 | Translation of ORF number 47 in reading frame 1 on the direct |
| | strand |
| | IRPETLRQAGAWRYGRLLPLPTIDNAPGCWGQERRPLSAERRTGECVSVCRVCVCVRWGPGGGW |
| | GPSPFDLPAWLGAGNSPGLTLPTFCSWCWGWGGVGKRLFALAPKAGCAPAAFSPRPHPARNPRP |
| | EIWDSFLIVPSLLS |
| 112 | ORF number 48 in reading frame 1 on the direct strand extends |
| | from base 57937 to base 58194 |
| | GAGTTAGTTGTGGTATTATCAAACCCAGGGCCTCTTAGTGAGTTCTGGGCACCCAGTGGTCAAA |
| | TTGCTAGAAGCATGTGCAGGAATGACCTCTCTGCTAAGAATAAAGTGGACTCTATAGGAAACAA |
| | TTTGCATGTGTGGGGGGTGGTATGGGAGACTATCCCAGGTGGTCCTCCTGGTGGAGGAGGTGAG |
| | GGAATCATGCAGGAGAGAACCCCAGGGAGAAGGGGAGAGTCCTTCATGCATTTTACCAGTGTTT |
| | AG |
| 113 | Translation of ORF number 48 in reading frame 1 on the direct |
| | strand |
| | ELVVVLSNPGPLSEFWAPSGQIARSMCRNDLSAKNKVDSIGNNLHVWGVVWETIPGGPPGGGGE |
| | GIMQERTPGRRGESFMHFTSV |
| 114 | ORF number 49 in reading frame 1 on the direct strand extends |
| | from base 58198 to base 58467 |
| | GCACCTACTCTGTGCTTTCCCCCAGTCTCTGTCCTGGGCTCTTCCCCGTGCAGGCTGGGAGGGT |
| | GGGGTTCTGGGTTTGTTTCCATAAGACATCATCGTCTCTTTTTTATTATAGGCCGGGTCCAGGG |
| | TGTCCACTGGGCCCAGCTGGGATCTGCCTACTCTGCCATGGCTAGCAGCTGCAGCCAGCTCTCC |
| | AGTGGGCAAGGAGGTCTTGGCATGAGTGTTACGTGCCATTTGGTACTGGGTCTTCAGTCCGCTC |
| | TCCTAAGAGGTTAA |
| 115 | Translation of ORF number 49 in reading frame 1 on the direct |
| | strand |
| | APTLCFPPVSVLGSSPCRLGGWGSGFVSIRHHRLFFIIGRVQGVHWAQLGSAYSAMASSCSQLS |
| | SGQGGLGMSVTCHLVLGLQSALLRG |
| 116 | ORF number 50 in reading frame 1 on the direct strand extends |
| | from base 59461 to base 59850 |
| | GGCACTGAGTTGTTAGACCCAAGGTTAAACAGTGGTAAGTCAAGTCAGCTGACACCCTCCCAGG |
| | GCTCCTCCCACGAGACCATGCCGTCCTGTGTGTTTGTGCACACACGTGTGTGTTTGTGCACACA |
| | CGTGTGTGTTTGCCTGGGAGTGAGTGCGGAGGTACAGCAGCATCTTATGCATTTTCTTTGCCCT |
| | GAGGCGCTGCGTGTCAGCTTTGTGTATCTCAGATTCTCATCTGCCCTCACTTCTTTCTCTAGAC |
| | CTCTGGCTTCAGCCCCTTGGGTCTCCCTGGACAGGGGGGGATGTGGCTGCGTCCTTCCTATCGG |
| | GCTGCTCTCATGTCATTGTGGGTCCTGTGGTTTCCCTGGAGGAAGCCCAGCTCCGAGTGGGGCC |
| | TGTTAA |
| 117 | Translation of ORF number 50 in reading frame 1 on the direct |
| | strand |
| | GTELLDPRLNSGKSSQLTPSQGSSHETMPSCVFVHTRVCLCTHVCVCLGVSAEVQQHLMHFLCP |
| | EALRVSFVYLRFSSALTSFSRPLASAPWVSLDRGGCGCVLPIGLLSCHCGSCGFPGGSPAPSGA |
| | C |
| 118 | ORF number 51 in reading frame 1 on the direct strand extends |
| | from base 60442 to base 60786 |
| | CCCGGCTGTCCACCTGTCCATGTCCAAGAGGCCCCGTGGGAACTTTCTGTAGGGGATAGTGTCT |
| | GTTGGGGCGAAGAGGGCTGTGGCTGGAAAGTCCTTACTCCCAGCGTGTTTGCCTGGCAGGGGGA |
| | CCCCATTCCTGAGGAACTCTATGAGATGCTGAGTGACCACTCGATCCGCTCCTTCGATGACCTC |
| | CAGCGCCTGCTGCACGGAGACTCCGTAGGTAAATTGAATCCTCGCCCAGGGCTCTGGCCCTCCA |
| | CTGAGTCCTCGCGTGCCAGGGGGTGGGGAGTGGGTGCCGGGCAAGGGCCATCCTCTCTTTTGTG |
| | CCATCCAGAGACCTGTGGCAGCTGA |
| 119 | Translation of ORF number 51 in reading frame 1 on the direct |
| | strand |
| | PGCPPVHVQEAPWELSVGDSVCWGEEGCGWKVLTPSVFAWQGDPIPEELYEMLSDHSIRSFDDL |
| | QRLLHGDSVGKLNPRPGLWPSTESSRARGWGVGAGQGPSSLLCHPETCGS |
| 120 | ORF number 52 in reading frame 1 on the direct strand extends |
| | from base 60787 to base 61305 |
| | GGGAGGACTTGGCCACACCTGTCTGGGGCAGGGCTGAGTAGGCGGACGGGCTGGTACCTAGGGT |
| | GTGAGGTGTGGCAGGAGAAGCATCCACATGTGGCTCTGGCTTGGGGTAGAGGGTGGGGCTGTGG |
| | GAGGGGAGGCAGGCAGGGAGAAGGTGCCCAGGGCATCTGCACCCTGAGTATCCAGGTGTGGACT |
| | CAGCCAGGGAGGGTGGTGCTGGAGGAGCCACCTCCCTGTCTCTCTGGCCAAAGGCCCGCTCTAC |
| | AAGGTCTCCCGGGGACACCTGGCCGGGACCAGTGGGCAGCCCTGCCCGTGCCCAAGAGGGCACT |
| | CAGAGAATGGGCACGTGCTTGGTGGCACACACGTGGCAGGGCTGGCGGGCTGTGTCGGGAATGT |
| | ATTTATAAACGCTGTCTTCAGAGCAAATTCCATTCTATTCTAACCTCTGGCCTGTTCCCTGGAG |
| | CCCTGGTCAGCACCCCCCTGCACCCCCAGCTCCCCTTCCCTCTGGGGTTTTGTCTCTTTGTCAC |
| | TTTGTAA |
| 121 | Translation of ORF number 52 in reading frame 1 on the direct |
| | strand |
| | GRTWPHLSGAGLSRRTGWYLGCEVWQEKHPHVALAWGRGWGCGRGGRQGEGAQGICTLSIQVWT |
| | QPGRVVLEEPPPCLSGQRPALQGLPGTPGRDQWAALPVPKRALREWARAWWHTRGRAGGLCREC |
| | IYKRCLQSKFHSILTSGLFPGALVSTPLHPQLPFPLGFCLFVTL |
| 122 | ORF number 53 in reading frame 1 on the direct strand extends |
| | from base 61306 to base 61710 |
| | TCCTTGCCCAGACTGCTATCTACGGGGGACAGCATTTCCTGCCTTTGTTTCCTCTCCCAGTTGG |
| | GCCCCTGGCTCCCTCTCAAAAGCATTCCCCGGGCCCTTTCAAACCCGCCTAGGCCGGGGGCTGA |
| | TGATGCAGGCAGGAGGGGGCCCCAGCTGGGCCCACCTATTGTTCACCAGGCCCCCCACCCGATG |
| | TCTCCCACACCCCCACCCCATGCCCGACTGGCCAGCCCTGGCCAACACAATGGGGCAACTTCCA |
| | AATTTAGCTTTTCTGCTGTTTCTTTCCAAGGTCCTTCGCCCCCACCCTCATATTGCCCCTCCAC |
| | ACCCCGGGTGGGGGTCGGGTCGGAGAAGACGAGGTTTTCAATAGCAGGCCTGTTTCGAGGCAAC |
| | CATGTGGCTATTTTTTCCTAA |
| 123 | Translation of ORF number 53 in reading frame 1 on the direct |
| | strand |
| | SLPRLLSTGDSISCLCFLSQLGPWLPLKSIPRALSNPPRPGADDAGRRGPQLGPPIVHQAPHPM |
| | SPTPPPHARLASPGQHNGATSKFSFSAVSFQGPSPPPSYCPSTPRVGVGSEKTRFSIAGLFRGN |
| | HVAIFS |
| 124 | ORF number 54 in reading frame 1 on the direct strand extends |
| | from base 61879 to base 62169 |
| | ACAGGGCCCCTTGGGAGCCCACAGGGACGAGCAATAGTTTTGTCATGGGCAGTGGCAGTGGGAT |
| | GGGGAGACAGTGTGACCCTGAGATGCTGTGTGGAGGGGGACAGAGCTTGTCCCCGACACCCTTC |
| | AGTGTATTTGCTGGCTTTCAGCCATCAGAGAGCTAGAAGAGTCTGCCCACCATTCAACGTCAAG |
| | CTCAAAGTTCCCCTGTCCAGCCCTCACTTTCCGCAGCCGGCTTCCGGCTGCCTCTACCCAGAGG |
| | GATGTCTCCAAGGAGTGCTGATGGTGCTGAGATGA |
| 125 | Translation of ORF number 54 in reading frame 1 on the direct |
| | strand |
| | TGPLGSPQGRAIVLSWAVAVGWGDSVTLRCCVEGDRACPRHPSVYLLAFSHQRARRVCPPFNVK |
| | LKVPLSSPHFPQPASGCLYPEGCLQGVLMVLR |
| 126 | ORF number 55 in reading frame 1 on the direct strand extends |
| | from base 62218 to base 62616 |
| | ATGTACAGCTTAGGGCAGGCATGGGGCAAAAGGTCAGAGGGAGAGAGACAGAAACACAATGAGG |
| | GACTGGGAGATGGAGAGAGACCAAGACCTAGAAGGACGCTGGGTGAGGGCTCCCCTATCCCAGC |
| | AGTTCCAGctccctacctctctctgcctttagtccccaccccaccccaccccacccctctcctt |
| | cccaccctctctcccgcccaacTGAACCATTGTCAGGGGCTCCACAGGGGCTGTGTCCAGGGCA |
| | TGCTGGTCCCCCCTGGGGACTATGGGAATTTCTCCATTCAGCACTTCCTATGGGAACGCTGGGT |
| | GGAGGGGCACTGGAAAGTGGCCTCAGAGCTCTGGGTCCTTGCCCTGCCCTGGAGGCCGAGGAGG |
| | GTTCGCTTACAGTAG |
| 127 | Translation of ORF number 55 in reading frame 1 on the direct |
| | strand |
| | MYSLGQAWGKRSEGERQKHNEGLGDGERPRPRRTLGEGSPIPAVPAPYLSLPLVPTPPHPTPLL |
| | PTLSPAQLNHCQGLHRGCVQGMLVPPGDYGNFSIQHFLWERWVEGHWKVASELWVLALPWRPRR |
| | VRLQ |
| 128 | ORF number 56 in reading frame 1 on the direct strand extends |
| | from base 62677 to base 62925 |
| | AGAGCCCAGAGTGGGGCTGAAGGCCCTCCGAGGGTACAGTCTGGGCCCCATCACCTCCTGAACC |
| | CCATGGCCACCCTGGGGTTTGCCTGGAGGGCGCCTCCTCAGAGGCAGGGAGCCAGAAGGGGAGT |
| | ATGTTCTCTGGAGTGGGGTCCCAGTGAGGGGCAAGAGGCAATCCTTCCGTCTGTCCCAGAGCCC |
| | CCACTGGAGTCCCCAGCCCGTGGTATGACCAGCCAGCACTTGTCACAGTGCTTCTGA |
| 129 | Translation of ORF number 56 in reading frame 1 on the direct |
| | strand |
| | RAQSGAEGPPRVQSGPHHLLNPMATLGFAWRAPPQRQGARRGVCSLEWGPSEGQEAILPSVPEP |
| | PLESPARGMTSQHLSQCF |
| 130 | ORF number 57 in reading frame 1 on the direct strand extends |
| | from base 63295 to base 63612 |
| | ccctattttataaaattggagactggagcccagagaagggaaagaagtggctgtggtgacacag |
| | ctagcatgtggtacggctgggatcccaaTAGCTCTTCTCAGTGCCGCCTGCTGTGTGTCTCTGC |
| | TGTGGCTAAGGGCTGTGGCCTGGGACAGCAGGCATGGAGCAAGCCTGGGACCTGCCTCCTGCTG |
| | TACTGCAGAAACCAAAAGGAGAATGTAGATCAGGGAAGGCAAGTGCCCACTCCACGCCCCTCTT |
| | CCTCTGTGCCCACCTGCAGTCCCCAAAACACTGTAGACAGTGGCTGGGGGGCCTCCAGGTAA |
| 131 | Translation of ORF number 57 in reading frame 1 on the direct |
| | strand |
| | PYFIKLETGAQRRERSGCGDTASMWYGWDPNSSSQCRLLCVSAVAKGCGLGQQAWSKPGTCLLL |
| | YCRNQKENVDQGRQVPTPRPSSSVPTCSPQNTVDSGWGASR |
| 132 | ORF number 58 in reading frame 1 on the direct strand extends |
| | from base 63946 to base 64236 |
| | AATGGATGGGGGCTGGCGGAAGGAAACTGGCATTTACAACATGCAGCAGCCTCTGAATTACCTC |
| | ACTTGATCCTGACAGTGGTTCTTGGGTGTAGACCTCATCACCCCCACTTGCACAGGGGGAAACA |
| | GATTCAGAACCCATCAGCGACCTGCCCAAATACCATGGCTGATAACAGCCAGTACTTAAAACCT |
| | CCCCTGACGTGGAAGGAAGAGGGAATCGGCCAACCGTTTTGGAGGTGCTCCACTTCCTGCCGGC |
| | TAGGGGCCCTGAGCAGCCCTCCACCCCACTCCTGA |
| 133 | Translation of ORF number 58 in reading frame 1 on the direct |
| | strand |
| | NGWGLAEGNWHLQHAAASELPHLILTVVLGCRPHHPHLHRGKQIQNPSATCPNTMADNSQYLKP |
| | PLTWKEEGIGQPFWRCSTSCRLGALSSPPPHS |
| 134 | ORF number 59 in reading frame 1 on the direct strand extends |
| | from base 64288 to base 64677 |
| | TCGCGGAGTGTAAAACGCGCACTGAGGTGTTTGAGATCTCCCGGCGCCTGGTTGACCGCACCAA |
| | CGCCAACTTCCTGGTGTGGCCGCCCTGCGTGGAGGTGCAGCGCTGCTCCGGCTGCTGCAACAAT |
| | CGCAACGTGCAGTGCCGCCCCACCCAGGTGCAGCTGCGACATGTCCAGGTGTGCAGGCCCCACG |
| | TCCCCTCCTGGGCTGGCCCAGCTGAGAGCAGGGGCTGCCCCTCTGGGGCTGGCACTCACGGACC |
| | AGGCTCTTGAATGCGTCAAAAGGCATTCCTGGCAGGGTGTGGGCTCAGTCCAGAGAAGGCGCTC |
| | TCAGGAAGCTCTCCGGACAGGTGTGCGGAGGCTGCCCAAGAATCCTCTATGGCCTCCCAAGCCA |
| | CTGTGA |
| 135 | Translation of ORF number 59 in reading frame 1 on the direct |
| | strand |
| | SRSVKRALRCLRSPGAWLTAPTPTSWCGRPAWRCSAAPAAATIATCSAAPPRCSCDMSRCAGPT |
| | SPPGLAQLRAGAAPLGLALTDQALECVKRHSWQGVGSVQRRRSQEALRTGVRRLPKNPLWPPKP |
| | L |
| 136 | ORF number 60 in reading frame 1 on the direct strand extends |
| | from base 65287 to base 65886 |
| | TCTGGTGACTTCACCACGCCCCCTCCCCTGCGGTCAGCTGTGGCCCTTCCTCTTGCCCACCTTC |
| | CATCCCAGGGCTGGGCCCTGAGCCCGAGATTACGAGTGTCACTCTCCACCCCACCTCCCACTGC |
| | CATGGTATCTCCTGTCCCCAATGCTTCCAGCTCTATGGATGGACACCTGACAGCTGACCTCCCC |
| | CTTCCCGCCTCCCTCCTGGATAAAGCCTCCCCTCACTTCCTTCCAGACAACCATCTCCCGCCTC |
| | TGCCACAGCCCCTGACCTTGGCTGGCGCTCCAGGAATGAGGACACCACAGGCTCCACGCTCCAC |
| | CCGGAAATGCCTTTCTCCCTCTCTGAGAGCACCGAGGGGGGCTGTGGCCAAGCTGGAGGCCAGG |
| | TCGGGAGGGCTTGTTTTGATGGAAAAGCTACAAGAAGGGCAGAGGGCAAGGTCCTGCTATTGTT |
| | TTGGCCGCAGTGTCTGCACTGCTGCTCTTCAGGCTTTCGAGGAAAGATTCCCCACAGAGGACGC |
| | TGGGGTGGGAAGAGAAGGCAGGCAGCTACCTCAGCCCCTGCCCAAGTGGTCTTACAGAGGCACT |
| | TGGGTGGTTCTGTCCTCCAGGTGA |
| 137 | Translation of ORF number 60 in reading frame 1 on the direct |
| | strand |
| | SGDFTTPPPLRSAVALPLAHLPSQGWALSPRLRVSLSTPPPTAMVSPVPNASSSMDGHLTADLP |
| | LPASLLDKASPHFLPDNHLPPLPQPLTLAGAPGMRTPQAPRSTRKCLSPSLRAPRGAVAKLEAR |
| | SGGLVLMEKLQEGQRARSCYCFGRSVCTAALQAFEERFPTEDAGVGREGRQLPQPLPKWSYRGT |
| | WVVLSSR |
| 138 | ORF number 61 in reading frame 1 on the direct strand extends |
| | from base 65995 to base 66225 |
| | CCCGAAGCCCAGGGAGTTCCCAGGAGCAGCGAGGTAACCTTCAGTCCAGGGTTGGTCTCTGATT |
| | GCTCAGCCTGGCCAGCCCCTTCTCCTGTGGCAGCTGCCGGGTGGGGGGAAATTGGATCAGGCAT |
| | GCGCCCCACCCCCcactcctggttaaattcatctgaagctttccatctcacagaacaatccaga |
| | ttcatccccccgactgcaaggccctatatgaggatgtag |
| 139 | Translation of ORF number 61 in reading frame 1 on the direct |
| | strand |
| | PEAQGVPRSSEVTFSPGLVSDCSAWPAPSPVAAAGWGEIGSGMRPTPHSWLNSSEAFHLTEQSR |
| | FIPPTARPYMRM |
| 140 | ORF number 62 in reading frame 1 on the direct strand extends |
| | from base 67639 to base 67965 |
| | TCAGAACCCTGGGCTAAAATTTCTGCTCTGTCACTTGTGAGTTGTACGACAACCTTGAGCTGGC |
| | TCGGGCTTTGCCAGTCCAGTGTCCTGCTGGTGGCCTGGTCTCTGGATCAGAAACTCCAGGCCCT |
| | CAATGGTTCTTCTGGGTACAAAGGTCCCAAGTCCCTGAATTGCAGAGATAGGGTAACTACTTTA |
| | TGGGAGCTTGTGTCTGCAAGGTGGGAGGTCAAGTGTTTAACCCAAAGAGTGGGGTGGGCCTTGA |
| | GCTTGGCAGAGAAAGCTTTCATTTTCTACTTGGGGGCCCAGGAGGAAGAGAGATGTAAGCGCAA |
| | ACCTTGA |
| 141 | Translation of ORF number 62 in reading frame 1 on the direct |
| | strand |
| | SEPWAKISALSLVSCTTTLSWLGLCQSSVLLVAWSLDQKLQALNGSSGYKGPKSLNCRDRVTTL |
| | WELVSARWEVKCLTQRVGWALSLAEKAFIFYLGAQEEERCKRKP |
| 142 | ORF number 63 in reading frame 1 on the direct strand extends |
| | from base 68611 to base 68883 |
| | gtctgtgggcggatggggctcagctgggtggttctactgctgtctctcatagtttcggtcagtc |
| | atctggaggccacactgggacagctgggcctctgtcattcagggcctctcttttccatatggtc |
| | tccccagcagggtaaccagacttcttatgtggcggcacagggctccacaaagtgcaaaggtggg |
| | acctaccaggcctttttaggcttatgcctggacctggcacagcactgctctgcctccttttatt |
| | gTTTAACAGatagatag |
| 143 | Translation of ORF number 63 in reading frame 1 on the direct |
| | strand |
| | VCGRMGLSWVVLLLSLIVSVSHLEATLGQLGLCHSGPLFSIWSPQQGNQTSYVAAQGSTKCKGG |
| | TYQAFLGLCLDLAQHCSASFYCLTDR |
| 144 | ORF number 64 in reading frame 1 on the direct strand extends |
| | from base 69562 to base 69948 |
| | GCGTGGCATGGAGTTCCTAGGCTGCTTCTGACCCCGTGTTCCTCTGCTTACCTTACAGGGTTAT |
| | TTAATATGGTATTTGCTGTATTGCCCCCATGGGGTCCTTGGAGTGATAATATTGTTCCCCTCGT |
| | CCGTCTGTCTCGATGCCTGATTCGGACGGCCAATGGTGCTTCCCCGCCCCTCCACGCGTCCGTC |
| | CACCCCTCTGCCAGTGGGTCTCCCCTCAGTGGCscadmCTCAGGGGCTGCGAGGAGCGCTACGC |
| | GCCTGGTCCCGTCCCGCCTCAGCTCGGCGGCCGCCGGGAGCCCGCACCGAGCCGGCTCCTGGGA |
| | GGGCCGGCCCCTCTCGGGCCTCCAACGAGGAGCAGGAAGGAGGCGGCGGCGGCGGCGAAGGGGT |
| | TAA |
| 145 | Translation of ORF number 64 in reading frame 1 on the direct |
| | strand |
| | AWHGVPRLLLTPCSSAYLTGLFNMVFAVLPPWGPWSDNIVPLVRLSRCLIRTANGASPPLHASV |
| | HPSASGSPLSGXXSGAARSATRLVPSRLSSAAAGSPHRAGSWEGRPLSGLQRGAGRRRRRRRRG |
| 146 | ORF number 65 in reading frame 1 on the direct strand extends |
| | from base 70192 to base 70821 |
| | TGCCCCCCGGGCCGCGGCCAGCTGTTGGGGCGGGGGGCGCCGGCCGGCCCCAGCTGCCGCCTCG |
| | CCGCggggcctgggggctgggccctgtgccagggcgtcctgggAACGGCGGCGCCCCAGCCGCT |
| | GCTCTCCGCAGCCCACCCCGCCCGGCCCCCCGACTCGCTCACTCACCCCACGCATGCACACTCT |
| | TGGCCGGAGGCGATGCTGCGCTCCGGCGGGCGGGCGCGCAGGGCGACGGGCACGCACTGGCGCG |
| | GCCGGGTcgcgcgcccgccgccacgcccgtgcacatgcgggacacacgcgcgcgcactacacac |
| | acacacgcatggtccccgcacacacggcttgagcacacgtgcgcgcacacccacgcacgcacAG |
| | CCTAGCGCCAGGTGCCCACCCCCGCGCCACAGGTGGGCCCACGGTAGGCCCTGGAACCTCGTCA |
| | ACTCTAGTGACTCTGTTTCCTTCTTGGGTGTTCTGAGGGAGGGGAAACAGGAACCCTCCCTCGG |
| | GTCCCTCTCCACAGCACCCATGGGTGTGTTTTTTTTTTTTTTTGGTCAGGTCAGTTCCACACCC |
| | TTTGCGCATTACCCTTCTATGATTGCTTTCTTTCAGCCACTCCCATGTGGCTGA |
| 147 | Translation of ORF number 65 in reading frame 1 on the direct |
| | strand |
| | CPPGRGQLLGRGAPAGPSCRLAAGPGGWALCQGVLGTAAPQPLLSAAHPARPPDSLTHPTHAHS |
| | WPEAMLRSGGRARRATGTHWRGRVARPPPRPCTCGTHARALHTHTHGPRTHGLSTRARTPTHAQ |
| | PSARCPPPRHRWAHGRPWNLVNSSDSVSFLGVLREGKQEPSLGSLSTAPMGVFFFFFGQVSSTP |
| | FAHYPSMIAFFQPLPCG |
| 148 | ORF number 66 in reading frame 1 on the direct strand extends |
| | from base 71266 to base 71607 |
| | AGGGAAAACCTATCGACATGGGGACCAGTCCAGGAAGGTGGACTTCAGGAGATCTTACTGGCAG |
| | AGGTGGCCTTGGGGCTGGCCACGTCTCAGGCCTGTGTGGCTGAGCCTCAGGTAGAGGGTAGAGG |
| | CCTCAGCAGCTGGGAAGGAGGGTTGGGACGGCTGAGGCAGGGCCTGGCAGGGGGTCAGCTGAGG |
| | CCTGTGAGGTTCCACCTCCATCAGCTGAACTGGCTTCAGGAGAGTGACTCCCACTGTCACGTGA |
| | GGCCTCCTGCCTTAGCACCCTTCTGCTGGGAAAGAGTGAAGGGGCACTACCGCCCTTCACCACC |
| | CAGCTTCCTTCTGGTTTGCTAA |
| 149 | Translation of ORF number 66 in reading frame 1 on the direct |
| | strand |
| | RENLSTWGPVQEGGLQEILLAEVALGLATSQACVAEPQVEGRGLSSWEGGLGRLRQGLAGGQLR |
| | PVRFHLHQLNWLQESDSHCHVRPPALAPFCWERVKGHYRPSPPSFLLVC |
| 150 | ORF number 67 in reading frame 1 on the direct strand extends |
| | from base 71608 to base 71940 |
| | TGCCTTAGGTGGTGGGAGACCAACTTGCTGGAATCTCCCAGCCCTAGACGTGTCTGCAAGGTTA |
| | AGATCAAACAGAATTTGGAGCTCTGGTGCAAAGCTAGGAACAGTGCGTGCATGCGCATgagaga |
| | gagagagagagagagagagagagagagagagagagagagagCCCTCTTCAGCAGGAGTGGTAAA |
| | GAGGTGTTTACCATGGGCCTCATAAATCTCTCAAAGTCTTCCCCCCCAACCCACCCGGTTGAAA |
| | TGCCCCTTCTAGACAGCTATTTTCATTTTCTGGTttatttagttgtttattatctgttttttct |
| | cactggagtgtaa |
| 151 | Translation of ORF number 67 in reading frame 1 on the direct |
| | strand |
| | CLRWWETNLLESPSPRRVCKVKIKQNLELWCKARNSACMRMRERERERERERERERALFSRSGK |
| | EVFTMGLINLSKSSPPTHPVEMPLLDSYFHFLVYLVVYYLFFLTGV |
| 152 | ORF number 68 in reading frame 1 on the direct strand extends |
| | from base 72526 to base 72789 |
| | CAGTTTTTCTGCTCAAGGGAGAGGTGGGGAGCCCAGTGGGAGGCTGGGCTCACATTAAGGAGGG |
| | GTGGGGGGGGGAGGGCCTCTGGAGCACTAGGAAAGGGAAATGGTAGGTGGGAAAGGCTGGGTCT |
| | AAATGGCTTCTGTGGTCTGCCCAGAGGAGGCGTCTTCAAAGGGCTTGGCTTTGGCGTTGAATCT |
| | AAATTAGGCCTGAGACTCTCAGGCAGGCTGGCGCTTGGAGGTATGGTCGGCTTCTGCCTCTGCC |
| | AACCATAG |
| 153 | Translation of ORF number 68 in reading frame 1 on the direct |
| | strand |
| | QFFCSRERWGAQWEAGLTLRRGGGGRASGALGKGNGRWERLGLNGFCGLPRGGVFKGLGFGVES |
| | KLGLRLSGRLALGGMVGFCLCQP |
| 154 | ORF number 69 in reading frame 1 on the direct strand extends |
| | from base 72790 to base 73128 |
| | ACAACGCCCCTGGGTGCTGGGGCCAAGAGCGACGTCCTCTCTCAGCTGAACGGCGCACTGGGGA |
| | gtgtgtatctgtgtgcagagtgtgtgtctgtgtgCGCTGGGGCCCAGGTGGAGGGTGGGGTCCA |
| | AGCCCCTTTGATCTGCCAGCATGGTTGGGAGCAGGTAATTCACCTGGCCTCACGCTTCCTACCT |
| | TCTGCAGCTGGTGTTGGGGGTGGGGTGGGGTGGGGAAGAGACTGTTTGCCTTGGCTCCCAAGGC |
| | TGGCTGTGCCCCAGCTGCCTTCTCGCCACGCCCTCACCCTGCTAGGAACCCCAGGCCTGAGATC |
| | TGGGACAGTTTCCTCATAG |
| 155 | Translation of ORF number 69 in reading frame 1 on the direct |
| | strand |
| | TTPLGAGAKSDVLSQLNGALGSVYLCAECVSVCAGAQVEGGVQAPLICQHGWEQVIHLASRFLP |
| | SAAGVGGGVGWGRDCLPWLPRLAVPQLPSRHALTLLGTPGLRSGTVSS |
| 156 | ORF number 70 in reading frame 1 on the direct strand extends |
| | from base 74314 to base 74541 |
| | GAAACAATTTGCATGTGTGGGGGGTGGTATGGGAGACTATCCCAGGTGGTCCTCCTGGTGGAGG |
| | AGGTGAGGGAATCATGCAGGAGAGAACCCCAGGGAGAAGGGGAGAGTCCTTCATGCATTTTACC |
| | AGTGTTTAGTGAGCACCTACTCTGTGCTTTCCCCCAGTCTCTGTCCTGGGCTCTTCCCCGTGCA |
| | GGCTGGGAGGGTGGGGTTCTGGGTTTGTTTCCATAA |
| 157 | Translation of ORF number 70 in reading frame 1 on the direct |
| | strand |
| | ETICMCGGWYGRLSQVVLLVEEVRESCRREPQGEGESPSCILPVFSEHLLCAFPQSLSWALPRA |
| | GWEGGVLGLFP |
| 158 | ORF number 71 in reading frame 1 on the direct strand extends |
| | from base 75868 to base 76191 |
| | GTGCGGAGGTACAGCAGCATCTTATGCATTTTCTTTGCCCTGAGGCGCTGCGTGTCAGCTTTGT |
| | GTATCTCAGATTCTCATCTGCCCTCACTTCTTTCTCTAGACCTCTGGCTTCAGCCCCTTGGGTC |
| | TCCCTGGACAGGGGGGGATGTGGCTGCGTCCTTCCTATCGGGCTGCTCTCATGTCATTGTGGGT |
| | CCTGTGGTTTCCCTGGAGGAAGCCCAGCTCCGAGTGGGGCCTGTTAAAGTGCTTATTAAGTTTC |
| | AAGTGTTTTTGGTAACAGGCCAGAGAGGCTCTAAAAATAGGGTTTGCCTGGGCACCGGGCATGG |
| | GTAA |
| 159 | Translation of ORF number 71 in reading frame 1 on the direct |
| | strand |
| | VRRYSSILCIFFALRRCVSALCISDSHLPSLLSLDLWLQPLGSPWTGGDVAASFLSGCSHVIVG |
| | PVVSLEEAQLRVGPVKVLIKFQVFLVTGQRGSKNRVCLGTGHG |
| 160 | ORF number 72 in reading frame 1 on the direct strand extends |
| | from base 76456 to base 76749 |
| | CAGACGCTGGCTGTCATCTGTCAGGTGTGGAGGAGAAGCATAAAGATTGTGGGGTTTCCCGGAA |
| | CCTGTAGTGTGATGAGGGAGATGGATGTATACAATCAATCAGAGCAAACTGGGGGTCCTCTTTG |
| | GAGGCGAGGGATACAGCATCCTCTCTGGGTCTTCAAGGCTTCGGCAGATTCTGGCCCTTGGGCC |
| | TTTGTGTTCCTGGTTCTCAGGCCTGGAATCTACCTCCTGCCCACCCCTAGCCCGGCTGTCCACC |
| | TGTCCATGTCCAAGAGGCCCCGTGGGAACTTTCTGTAG |
| 161 | Translation of ORF number 72 in reading frame 1 on the direct |
| | strand |
| | QTLAVICQVWRRSIKIVGFPGTCSVMREMDVYNQSEQTGGPLWRRGIQHPLWVFKASADSGPWA |
| | FVFLVLRPGIYLLPTPSPAVHLSMSKRPRGNFL |
| 162 | ORF number 73 in reading frame 1 on the direct strand extends |
| | from base 77218 to base 77469 |
| | GTATCCAGGTGTGGACTCAGCCAGGGAGGGTGGTGCTGGAGGAGCCACCTCCCTGTCTCTCTGG |
| | CCAAAGGCCCGCTCTACAAGGTCTCCCGGGGACACCTGGCCGGGACCAGTGGGCAGCCCTGCCC |
| | GTGCCCAAGAGGGCACTCAGAGAATGGGCACGTGCTTGGTGGCACACACGTGGCAGGGCTGGCG |
| | GGCTGTGTCGGGAATGTATTTATAAACGCTGTCTTCAGAGCAAATTCCATTCTATTCTAA |
| 163 | Translation of ORF number 73 in reading frame 1 on the direct |
| | strand |
| | VSRCGLSQGGWCWRSHLPVSLAKGPLYKVSRGHLAGTSGQPCPCPRGHSENGHVLGGTHVAGLA |
| | GCVGNVFINAVFRANSILF |
| 164 | ORF number 74 in reading frame 1 on the direct strand extends |
| | from base 77470 to base 77925 |
| | CCTCTGGCCTGTTCCCTGGAGCCCTGGTCAGCACCCCCCTGCACCCCCAGCTCCCCTTCCCTCT |
| | GGGGTTTTGTCTCTTTGTCACTTTGTAATCCTTGCCCAGACTGCTATCTACGGGGGACAGCATT |
| | TCCTGCCTTTGTTTCCTCTCCCAGTTGGGCCCCTGGCTCCCTCTCAAAAGCATTCCCCGGGCCC |
| | TTTCAAACCCGCCTAGGCCGGGGGCTGATGATGCAGGCAGGAGGGGGCCCCAGCTGGGCCCACC |
| | TATTGTTCACCAGGCCCCCCACCCGATGTCTCCCACACCCCCACCCCATGCCCGACTGGCCAGC |
| | CCTGGCCAACACAATGGGGCAACTTCCAAATTTAGCTTTTCTGCTGTTTCTTTCCAAGGTCCTT |
| | CGCCCCCACCCTCATATTGCCCCTCCACACCCCGGGTGGGGGTCGGGTCGGAGAAGACGAGGTT |
| | TTCAATAG |
| 165 | Translation of ORF number 74 in reading frame 1 on the direct |
| | strand |
| | PLACSLEPWSAPPCTPSSPSLWGFVSLSLCNPCPDCYLRGTAFPAFVSSPSWAPGSLSKAFPGP |
| | FQTRLGRGLMMQAGGGPSWAHLLFTRPPTRCLPHPHPMPDWPALANTMGQLPNLAFLLFLSKVL |
| | RPHPHIAPPHPGWGSGRRRRGFQ |
| 166 | ORF number 75 in reading frame 1 on the direct strand extends |
| | from base 78691 to base 78993 |
| | ACCATTGTCAGGGGCTCCACAGGGGCTGTGTCCAGGGCATGCTGGTCCCCCCTGGGGACTATGG |
| | GAATTTCTCCATTCAGCACTTCCTATGGGAACGCTGGGTGGAGGGGCACTGGAAAGTGGCCTCA |
| | GAGCTCTGGGTCCTTGCCCTGCCCTGGAGGCCGAGGAGGGTTCGCTTACAGTAGCAAAAGGGAA |
| | CGGTTATTTTTAACTCCATTGACATGGGTTCTGTCCAAAAATGTGGCTGAAGAGCCCAGAGTGG |
| | GGCTGAAGGCCCTCCGAGGGTACAGTCTGGGCCCCATCACCTCCTGA |
| 167 | Translation of ORF number 75 in reading frame 1 on the direct |
| | strand |
| | TIVRGSTGAVSRACWSPLGTMGISPFSTSYGNAGWRGTGKWPQSSGSLPCPGGRGGFAYSSKRE |
| | RLFLTPLTWVLSKNVAEEPRVGLKALRGYSLGPITS |
| 168 | ORF number 76 in reading frame 1 on the direct strand extends |
| | from base 80761 to base 80985 |
| | GAGCAGGGGCTGCCCCTCTGGGGCTGGCACTCACGGACCAGGCTCTTGAATGCGTCAAAAGGCA |
| | TTCCTGGCAGGGTGTGGGCTCAGTCCAGAGAAGGCGCTCTCAGGAAGCTCTCCGGACAGGTGTG |
| | CGGAGGCTGCCCAAGAATCCTCTATGGCCTCCCAAGCCACTGTGACAAAAAGTCACAGGCAGAC |
| | CTCCAGACAGGCTGGGTATGGGACATTAAGTAA |
| 169 | Translation of ORF number 76 in reading frame 1 on the direct |
| | strand |
| | EQGLPLWGWHSRTRLLNASKGIPGRVWAQSREGALRKLSGQVCGGCPRILYGLPSHCDKKSQAD |
| | LQTGWVWDIK |
| 170 | ORF number 77 in reading frame 1 on the direct strand extends |
| | from base 81946 to base 82179 |
| | TGGAAAAGCTACAAGAAGGGCAGAGGGCAAGGTCCTGCTATTGTTTTGGCCGCAGTGTCTGCAC |
| | TGCTGCTCTTCAGGCTTTCGAGGAAAGATTCCCCACAGAGGACGCTGGGGTGGGAAGAGAAGGC |
| | AGGCAGCTACCTCAGCCCCTGCCCAAGTGGTCTTACAGAGGCACTTGGGTGGTTCTGTCCTCCA |
| | GGTGAGGAAGATCGAGATTGTACGGAAGAAGCCAAGCTTTAA |
| 171 | Translation of ORF number 77 in reading frame 1 on the direct |
| | strand |
| | WKSYKKGRGQGPAIVLAAVSALLLFRLSRKDSPQRTLGWEEKAGSYLSPCPSGLTEALGWFCPP |
| | GEEDRDCTEEAKL |
| 172 | ORF number 78 in reading frame 1 on the direct strand extends |
| | from base 82474 to base 82701 |
| | ggatgtagcccccagttggccctttggtcttgctgccaaccaatcccccctcactgtgacaccc |
| | cagccagcctggcctttttgaatggccagctacatttctgcctcagggcctttgcacatgccac |
| | tctgtctgaaactcacttctctcagctcttcacaagcctactccttctcttcatttggatctta |
| | gctcagaagtcatctcctcctagaagtctgccctga |
| 173 | Translation of ORF number 78 in reading frame 1 on the direct |
| | strand |
| | GCSPQLALWSCCQPIPPHCDTPASLAFLNGQLHFCLRAFAHATLSETHFSQLFTSLLLLFIWIL |
| | AQKSSPPRSLP |
| 174 | ORF number 79 in reading frame 1 on the direct strand extends |
| | from base 84400 to base 84645 |
| | gggtttctggctattttcatatactatctcctaatcctaggaggccagggctgctggcatctcc |
| | attttagagatgtggaaattgaggcacagggagtttatatgacttgcccaaaccacatgactaa |
| | cacgtgggagagcccagatttgaacccaggtGGTCTGGCCCACCATCTGAGCTCTGGACTGCCC |
| | CACTGTGCCGTTACTCTAAGTGGCGAGGGTAAGGCAGACGTCAGGCGCAACTGA |
| 175 | Translation of ORF number 79 in reading frame 1 on the direct |
| | strand |
| | GFLAIFIYYLLILGGQGCWHLHFRDVEIEAQGVYMTCPNHMTNTWESPDLNPGGLAHHLSSGLP |
| | HCAVTLSGEGKADVRRN |
| 176 | ORF number 80 in reading frame 1 on the direct strand extends |
| | from base 85966 to base 86799 |
| | TTCGGACGGCCAATGGTGCTTCCCCGCCCCTCCACGCGTCCGTCCACCCCTCTGCCAGTGGGTC |
| | TCCCCTCAGTGGCscadmCTCAGGGGCTGCGAGGAGCGCTACGCGCCTGGTCCCGTCCCGCCTC |
| | AGCTCGGCGGCCGCCGGGAGCCCGCACCGAGCCGGCTCCTGGGAGGGCCGGCCCCTCTCGGGCC |
| | TCCAACGAGGAGCAGGAAGGAGGCGGCGGCGGCGGCGAAGGGGTTAAGGTGAAGGGCTTCGAGG |
| | CCGCGGCCGGGCCTTGGGCCGCAGCCAGCGCAGGTTGTTTTGACCACGGAGGAGCCGTCTCCGT |
| | CTCCTTTTGTTCTCGGGGCTCCTCGAGGGCCGCCGGCCGTCCGCCCTGGGGCCCCGCCCTTCCG |
| | CGGCCGTCCCCCGTGGCCCGCACCCGGGAGGGAGGACGCGGGGATCAGCCTGGCTGCCTGCAGT |
| | CCCCTCCCGACGCCCCCTCCTCTCCTCCTGCTGATGCCCCCCGGGCCGCGGCCAGCTGTTGGGG |
| | CGGGGGGCGCCGGCCGGCCCCAGCTGCCGCCTCGCCGCggggcctgggggctgggccctgtgcc |
| | agggcgtcctgggAACGGCGGCGCCCCAGCCGCTGCTCTCCGCAGCCCACCCCGCCCGGCCCCC |
| | CGACTCGCTCACTCACCCCACGCATGCACACTCTTGGCCGGAGGCGATGCTGCGCTCCGGCGGG |
| | CGGGCGCGCAGGGCGACGGGCACGCACTGGCGCGGCCGGGTcgcgcgcccgccgccacgcccgt |
| | gcacatgcgggacacacgcgcgcgcactacacacacacacgcatggtccccgcacacacggctt |
| | ga |
| 177 | Translation of ORF number 80 in reading frame 1 on the direct |
| | strand |
| | FGRPMVLPRPSTRPSTPLPVGLPSVAXXQGLRGALRAWSRPASARRPPGARTEPAPGRAGPSRA |
| | SNEEQEGGGGGGEGVKVKGFEAAAGPWAAASAGCFDHGGAVSVSFCSRGSSRAAGRPPWGPALP |
| | RPSPVARTREGGRGDQPGCLQSPPDAPSSPPADAPRAAASCWGGGRRPAPAAASPRGLGAGPCA |
| | RASWERRRPSRCSPQPTPPGPPTRSLTPRMHTLGRRRCCAPAGGRAGRRARTGAAGSRARRHAR |
| | AHAGHTRAHYTHTRMVPAHTA |
| 178 | ORF number 81 in reading frame 1 on the direct strand extends |
| | from base 87169 to base 87486 |
| | GCTTCCGAGGGGGCTCCCACCCCCCACTGTTCTGTGCTCTTTGCTGATCCCAGCCAGCACGCTG |
| | CAGAGAGGCTGGGTGACAGCTGGATAAGGCTTTCCCGCCTGCCCTTACCATTCCCAGCTTCATC |
| | CAGCACCTCCTCCTCCTTTCCCACAACTCCCTGGGTGTGTGTTTGGGGGGTGAGCCTATGGCAC |
| | AGAAACTGGTGCCTGTCTCCTCACTTTAATCACAGCATCCTTGGACACATGGCTCTCAGGAACC |
| | CACAGTTGTGTGGTGCTTTGCAGTTTACGAAGCACTTTCCTGCTAAGCCTTACTCTGAGTAA |
| 179 | Translation of ORF number 81 in reading frame 1 on the direct |
| | strand |
| | ASEGAPTPHCSVLFADPSQHAAERLGDSWIRLSRLPLPFPASSSTSSSFPTTPWVCVWGVSLWH |
| | RNWCLSPHFNHSILGHMALRNPQLCGALQFTKHFPAKPYSE |
| 180 | ORF number 82 in reading frame 1 on the direct strand extends |
| | from base 88375 to base 88617 |
| | TCCTCAGCCCAGGGTAGCCTAGAATGGCCACACTGCTCTTCACCAGGCATCCTCATTCGAGCCC |
| | CCCCGGCCCCCCATCTTGAGAGACAAGCATATCTTTCTTTTCCATGTCTTGGGCTGCCAATATT |
| | GGACAGGACAGAGGGGAAGAAACAGAAGGAAAATCAGATCGCAAGGCTTCTGTGTATCTTGAGC |
| | AGGCCTGGGCCTCAGTTGCCGCCGCGTGAGAATATGAGAAGGTTGGATTAG |
| 181 | Translation of ORF number 82 in reading frame 1 on the direct |
| | strand |
| | SSAQGSLEWPHCSSPGILIRAPPAPHLERQAYLSFPCLGLPILDRTEGKKQKENQIARLLCILS |
| | RPGPQLPPRENMRRLD |
| 182 | ORF number 83 in reading frame 1 on the direct strand extends |
| | from base 89983 to base 90402 |
| | gCAATCCAGGTTTCCTTGGCAGCTGAAGCTCTACAgtttctctgcctctccactgttgacattt |
| | ggggccagacagttcttgattgtgggggaggctgtcctgtgcatagtaggatgtttagcagcaa |
| | ccctggcctctacctactagacaccagtagcaggcctccagttgtgataaccaaaagtgcctcc |
| | agacctggccagtgtcccctgggggtcaCTCACTCCCTGCTCTATGACCTCCACTGGGTGAAGA |
| | GTGGACCTGAACTGAAAACAGTCCATGAAAGAGGGAGGGGCCCGCTCTGCTCCTTACCAGTCGT |
| | GTTGACCTTTAGCCATTTACTTAATTTTTCTAAGCCTCAGCTTCCTCATTTGGAAGACAGGGAT |
| | ACAAACAGTGACAGCCTCTTGATTGTATTTGATTGA |
| 183 | Translation of ORF number 83 in reading frame 1 on the direct |
| | strand |
| | AIQVSLAAEALQFLCLSTVDIWGQTVLDCGGGCPVHSRMFSSNPGLYLLDTSSRPPVVITKSAS |
| | RPGQCPLGVTHSLLYDLHWVKSGPELKTVHERGRGPLCSLPVVLTFSHLLNFSKPQLPHLEDRD |
| | TNSDSLLIVFD |
| 184 | ORF number 84 in reading frame 1 on the direct strand extends |
| | from base 90640 to base 90882 |
| | GGGAATCATGCAGGAGAGAACCCCAGGGAGAAGGGGAGAGTCCTTCATGCATTTTACCAGTGTT |
| | TAGTGAGCACCTACTCTGTGCTTTCCCCCAGTCTCTGTCCTGGGCTCTTCCCCGTGCAGGCTGG |
| | GAGGGTGGGGTTCTGGGTTTGTTTCCATAAGACATCATCGTCTCTTTTTTATTATAGGCCGGGT |
| | CCAGGGTGTCCACTGGGCCCAGCTGGGATCTGCCTACTCTGCCATGGCTAG |
| 185 | Translation of ORF number 84 in reading frame 1 on the direct |
| | strand |
| | GNHAGENPREKGRVLHAFYQCLVSTYSVLSPSLCPGLFPVQAGRVGFWVCFHKTSSSLFYYRPG |
| | PGCPLGPAGICLLCHG |
| 186 | ORF number 85 in reading frame 1 on the direct strand extends |
| | from base 90883 to base 91161 |
| | CAGCTGCAGCCAGCTCTCCAGTGGGCAAGGAGGTCTTGGCATGAGTGTTACGTGCCATTTGGTA |
| | CTGGGTCTTCAGTCCGCTCTCCTAAGAGGTTAATTGATTCATTATGCCACAAACAGCCTGGGAG |
| | ACCTGGCTGGGCACCCCCACTTCGGCTTCCTCTGCTGCTGCCTCTCCTGCCAACCCCAGACAGA |
| | ATTAGAATTAAAATCAAATCAAATGGCTACAACCCCCTCAGTTCACAGGTGATAGCCAGGACCC |
| | GAGAGGGGCAGCAACCAACCTGA |
| 187 | Translation of ORF number 85 in reading frame 1 on the direct |
| | strand |
| | QLQPALQWARRSWHECYVPFGTGSSVRSPKRLIDSLCHKQPGRPGWAPPLRLPLLLPLLPTPDR |
| | IRIKIKSNGYNPLSSQVIARTREGQQPT |
| 188 | ORF number 86 in reading frame 1 on the direct strand extends |
| | from base 93412 to base 93690 |
| | AGGGTGGGGCTGTGGGAGGGGAGGCAGGCAGGGAGAAGGTGCCCAGGGCATCTGCACCCTGAGT |
| | ATCCAGGTGTGGACTCAGCCAGGGAGGGTGGTGCTGGAGGAGCCACCTCCCTGTCTCTCTGGCC |
| | AAAGGCCCGCTCTACAAGGTCTCCCGGGGACACCTGGCCGGGACCAGTGGGCAGCCCTGCCCGT |
| | GCCCAAGAGGGCACTCAGAGAATGGGCACGTGCTTGGTGGCACACACGTGGCAGGGCTGGCGGG |
| | CTGTGTCGGGAATGTATTTATAA |
| 189 | Translation of ORF number 86 in reading frame 1 on the direct |
| | strand |
| | RVGLWEGRQAGRRCPGHLHPEYPGVDSAREGGAGGATSLSLWPKARSTRSPGDTWPGPVGSPAR |
| | AQEGTQRMGTCLVAHTWQGWRAVSGMYL |
| 190 | ORF number 87 in reading frame 1 on the direct strand extends |
| | from base 93691 to base 93933 |
| | ACGCTGTCTTCAGAGCAAATTCCATTCTATTCTAACCTCTGGCCTGTTCCCTGGAGCCCTGGTC |
| | AGCACCCCCCTGCACCCCCAGCTCCCCTTCCCTCTGGGGTTTTGTCTCTTTGTCACTTTGTAAT |
| | CCTTGCCCAGACTGCTATCTACGGGGGACAGCATTTCCTGCCTTTGTTTCCTCTCCCAGTTGGG |
| | CCCCTGGCTCCCTCTCAAAAGCATTCCCCGGGCCCTTTCAAACCCGCCTAG |
| 191 | Translation of ORF number 87 in reading frame 1 on the direct |
| | strand |
| | TLSSEQIPFYSNLWPVPWSPGQHPPAPPAPLPSGVLSLCHFVILAQTAIYGGQHFLPLFPLPVG |
| | PLAPSQKHSPGPFKPA |
| 192 | ORF number 88 in reading frame 1 on the direct strand extends |
| | from base 94081 to base 94554 |
| | CTTTTCTGCTGTTTCTTTCCAAGGTCCTTCGCCCCCACCCTCATATTGCCCCTCCACACCCCGG |
| | GTGGGGGTCGGGTCGGAGAAGACGAGGTTTTCAATAGCAGGCCTGTTTCGAGGCAACCATGTGG |
| | CTATTTTTTCCTAATCAACTTAACCTTTCCACAAAGCACATCTTTTCCCCATCTCCTCCCAACC |
| | AGGGACATTCCAGAAATGGCAGAGAGAAAGGAATGGAGCCAGAGGGACAGACAGACACACTGTT |
| | CGTGGGACAATAGGCTAGACGGAAGTGCATCAGTTTTAGGAAAGTCTGCTCTAAACAGGGCCCC |
| | TTGGGAGCCCACAGGGACGAGCAATAGTTTTGTCATGGGCAGTGGCAGTGGGATGGGGAGACAG |
| | TGTGACCCTGAGATGCTGTGTGGAGGGGGACAGAGCTTGTCCCCGACACCCTTCAGTGTATTTG |
| | CTGGCTTTCAGCCATCAGAGAGCTAG |
| 193 | Translation of ORF number 88 in reading frame 1 on the direct |
| | strand |
| | LFCCFFPRSFAPTLILPLHTPGGGRVGEDEVFNSRPVSRQPCGYFFLINLTFPQSTSFPHLLPT |
| | RDIPEMAERKEWSQRDRQTHCSWDNRLDGSASVLGKSALNRAPWEPTGTSNSFVMGSGSGMGRQ |
| | CDPEMLCGGGQSLSPTPFSVFAGFQPSES |
| 194 | ORF number 89 in reading frame 1 on the direct strand extends |
| | from base 94555 to base 94791 |
| | AAGAGTCTGCCCACCATTCAACGTCAAGCTCAAAGTTCCCCTGTCCAGCCCTCACTTTCCGCAG |
| | CCGGCTTCCGGCTGCCTCTACCCAGAGGGATGTCTCCAAGGAGTGCTGATGGTGCTGAGATGAG |
| | GGCCTCCAGGCTAGAGAAGGGAGCTGTAGTTGTGACCTTAGGAATAAATGTACAGCTTAGGGCA |
| | GGCATGGGGCAAAAGGTCAGAGGGAGAGAGACAGAAACACAATGA |
| 195 | Translation of ORF number 89 in reading frame 1 on the direct |
| | strand |
| | KSLPTIQRQAQSSPVQPSLSAAGFRLPLPRGMSPRSADGAEMRASRLEKGAVVVTLGINVQLRA |
| | GMGQKVRGRETETQ |
| 196 | ORF number 90 in reading frame 1 on the direct strand extends |
| | from base 94840 to base 95151 |
| | GGGCTCCCCTATCCCAGCAGTTCCAGctccctacctctctctgcctttagtccccaccccaccc |
| | caccccacccctctccttcccaccctctctcccgcccaacTGAACCATTGTCAGGGGCTCCACA |
| | GGGGCTGTGTCCAGGGCATGCTGGTCCCCCCTGGGGACTATGGGAATTTCTCCATTCAGCACTT |
| | CCTATGGGAACGCTGGGTGGAGGGGCACTGGAAAGTGGCCTCAGAGCTCTGGGTCCTTGCCCTG |
| | CCCTGGAGGCCGAGGAGGGTTCGCTTACAGTAGCAAAAGGGAACGGTTATTTTTAA |
| 197 | Translation of ORF number 90 in reading frame 1 on the direct |
| | strand |
| | GLPYPSSSSSLPLSAFSPHPTPPHPSPSHPLSRPTEPLSGAPQGLCPGHAGPPWGLWEFLHSAL |
| | PMGTLGGGALESGLRALGPCPALEAEEGSLTVAKGNGYF |
| 198 | ORF number 91 in reading frame 1 on the direct strand extends |
| | from base 95344 to base 95658 |
| | GGGGCAAGAGGCAATCCTTCCGTCTGTCCCAGAGCCCCCACTGGAGTCCCCAGCCCGTGGTATG |
| | ACCAGCCAGCACTTGTCACAGTGCTTCTGACTGTGCCTTCTCTTGCAGATGAAGACGGGGCTGA |
| | GTTGGACCTGAATTTGACTCAGTCCCATTCTGGAGGCAAGCTGGAGAGCTTATCCCGAGGGAGA |
| | AGGAGCCTAGGTAAGAATGAGGGTGCAAACGGGGGCCCCTCAAAGGTGGGGGCCAGGGAAGAAG |
| | AACTGAGCACACAGCCTGCCGGAGGCTGTGAGGGTGGGCCCTGTTTGTCCCACACTTAG |
| 199 | Translation of ORF number 91 in reading frame 1 on the direct |
| | strand |
| | GARGNPSVCPRAPTGVPSPWYDQPALVTVLLTVPSLADEDGAELDLNLTQSHSGGKLESLSRGR |
| | RSLGKNEGANGGPSKVGAREEELSTQPAGGCEGGPCLSHT |
| 200 | ORF number 92 in reading frame 1 on the direct strand extends |
| | from base 95944 to base 96174 |
| | GGGCTGTGGCCTGGGACAGCAGGCATGGAGCAAGCCTGGGACCTGCCTCCTGCTGTACTGCAGA |
| | AACCAAAAGGAGAATGTAGATCAGGGAAGGCAAGTGCCCACTCCACGCCCCTCTTCCTCTGTGC |
| | CCACCTGCAGTCCCCAAAACACTGTAGACAGTGGCTGGGGGGCCTCCAGGTAAGAGTCAGTGGC |
| | CTGAGTTCCACTCTTTGCTCTGTGAATTTGGGCATCTAA |
| 201 | Translation of ORF number 92 in reading frame 1 on the direct |
| | strand |
| | GLWPGTAGMEQAWDLPPAVLQKPKGECRSGKASAHSTPLFLCAHLQSPKHCRQWLGGLQVRVSG |
| | LSSTLCSVNLGI |
| 202 | ORF number 93 in reading frame 1 on the direct strand extends |
| | from base 96631 to base 97065 |
| | CAGCCAGTACTTAAAACCTCCCCTGACGTGGAAGGAAGAGGGAATCGGCCAACCGTTTTGGAGG |
| | TGCTCCACTTCCTGCCGGCTAGGGGCCCTGAGCAGCCCTCCACCCCACTCCTGACGGAGTTCCC |
| | TCTCCCTTCAGATTCCCAGCCGGTCGCCGAGCCAGCCATGATCGCGGAGTGTAAAACGCGCACT |
| | GAGGTGTTTGAGATCTCCCGGCGCCTGGTTGACCGCACCAACGCCAACTTCCTGGTGTGGCCGC |
| | CCTGCGTGGAGGTGCAGCGCTGCTCCGGCTGCTGCAACAATCGCAACGTGCAGTGCCGCCCCAC |
| | CCAGGTGCAGCTGCGACATGTCCAGGTGTGCAGGCCCCACGTCCCCTCCTGGGCTGGCCCAGCT |
| | GAGAGCAGGGGCTGCCCCTCTGGGGCTGGCACTCACGGACCAGGCTCTTGA |
| 203 | Translation of ORF number 93 in reading frame 1 on the direct |
| | strand |
| | QPVLKTSPDVEGRGNRPTVLEVLHFLPARGPEQPSTPLLTEFPLPSDSQPVAEPAMIAECKTRT |
| | EVFEISRRLVDRTNANFLVWPPCVEVQRCSGCCNNRNVQCRPTQVQLRHVQVCRPHVPSWAGPA |
| | ESRGCPSGAGTHGPGS |
| 204 | ORF number 94 in reading frame 1 on the direct strand extends |
| | from base 97066 to base 97338 |
| | ATGCGTCAAAAGGCATTCCTGGCAGGGTGTGGGCTCAGTCCAGAGAAGGCGCTCTCAGGAAGCT |
| | CTCCGGACAGGTGTGCGGAGGCTGCCCAAGAATCCTCTATGGCCTCCCAAGCCACTGTGACAAA |
| | AAGTCACAGGCAGACCTCCAGACAGGCTGGGTATGGGACATTAAGTAAAAGGCATTGCCTCATT |
| | CTTTACAGGGATAAAATCCCAAAATGTCTCTTGAAGAGACATGTCTACAAACATATTGGACCCT |
| | CAGGATGTTCTGGGTAG |
| 205 | Translation of ORF number 94 in reading frame 1 on the direct |
| | strand |
| | MRQKAFLAGCGLSPEKALSGSSPDRCAEAAQESSMASQATVTKSHRQTSRQAGYGTLSKRHCLI |
| | LYRDKIPKCLLKRHVYKHIGPSGCSG |
| 206 | ORF number 95 in reading frame 1 on the direct strand extends |
| | from base 98014 to base 98568 |
| | AGCCTCCCCTCACTTCCTTCCAGACAACCATCTCCCGCCTCTGCCACAGCCCCTGACCTTGGCT |
| | GGCGCTCCAGGAATGAGGACACCACAGGCTCCACGCTCCACCCGGAAATGCCTTTCTCCCTCTC |
| | TGAGAGCACCGAGGGGGGCTGTGGCCAAGCTGGAGGCCAGGTCGGGAGGGCTTGTTTTGATGGA |
| | AAAGCTACAAGAAGGGCAGAGGGCAAGGTCCTGCTATTGTTTTGGCCGCAGTGTCTGCACTGCT |
| | GCTCTTCAGGCTTTCGAGGAAAGATTCCCCACAGAGGACGCTGGGGTGGGAAGAGAAGGCAGGC |
| | AGCTACCTCAGCCCCTGCCCAAGTGGTCTTACAGAGGCACTTGGGTGGTTCTGTCCTCCAGGTG |
| | AGGAAGATCGAGATTGTACGGAAGAAGCCAAGCTTTAAGAAGGCCACAGTGACCCTGGAGGACC |
| | ACCTGGCGTGCAAGTGTGAGACGGTAGTGGCTGCACGACCTGTGACCCGAAGCCCAGGGAGTTC |
| | CCAGGAGCAGCGAGGTAACCTTCAGTCCAGGGTTGGTCTCTGA |
| 207 | Translation of ORF number 95 in reading frame 1 on the direct |
| | strand |
| | SLPSLPSRQPSPASATAPDLGWRSRNEDTTGSTLHPEMPFSLSESTEGGCGQAGGQVGRACFDG |
| | KATRRAEGKVLLLFWPQCLHCCSSGFRGKIPHRGRWGGKRRQAATSAPAQVVLQRHLGGSVLQV |
| | RKIEIVRKKPSFKKATVTLEDHLACKCETVVAARPVTRSPGSSQEQRGNLQSRVGL |
| 208 | ORF number 96 in reading frame 1 on the direct strand extends |
| | from base 102187 to base 103830 |
| | TATTGTTCCCCTCGTCCGTCTGTCTCGATGCCTGATTCGGACGGCCAATGGTGCTTCCCCGCCC |
| | CTCCACGCGTCCGTCCACCCCTCTGCCAGTGGGTCTCCCCTCAGTGGCscadmatctgtggcca |
| | gcctaattcaagaaagtcgtttggaagctcgaaaatattacggaaaggagccagatttgattgt |
| | tgttccttttacaaaaatgcagattcaaggcttgatgcagtttacagttttcccatcgccttgg |
| | ctcattttacaggaactttagataatcattatcctaagcataaattgcttcagttttttcaaca |
| | tcatgatccaatttttccttcaattgtgtcacatgctcctcttcctgctgttccaaatgttttt |
| | actgatggatctaataatggagtagctgtttatgcactcaataaaaaagtcaccaagagagtac |
| | agacacctccagcttcagctcaaatagttgagcttcgagcagtacataaggtgctgcttgattt |
| | tgcttctcagtcttttaatttattctctgacagccattatgtggttcgtgcagtcagaaattta |
| | gaaacagtaccttttattagcactagtaatcctgttattcaggatttgtttcttcagatacaac |
| | aggccattcagctgcgctgtaaaaaattttatattggccatattagagctcactctaatcttcc |
| | aggtcctttagcagcaggcaatcaaattgcagattctgccacgcagcttattgccttaactcaa |
| | atagaaaaagcacaaaaggctcatagcctccaccatcaaaatagccagagcctaagattacagt |
| | ataagatcctcagagaagcagcacgccagattataaaacaatgtccagattgctcgcatttaca |
| | acctgtgcctcattatggcattaaccctcgaggcttgcgtcccaatgatctgtggcaaatggat |
| | gttactcatatacctgaatttggaaaattaaaatacgtccatgtctctatagacacgttttctg |
| | gctttgtaatagcttctgctcaatcaggagaagctacatctcatgttattagacattgtcttgc |
| | tgcttttgccatgattggcactcctaaaaaacttaaaacagataatggctccggctacaccagt |
| | aaaaaatttgctttattttgtcaacaatttttaattaatcatgttactggcattccttacaatc |
| | cccagcgacaagggattgttgaacgtactcatggcacattaaaagtcattttacaaaaaataaa |
| | aaagggggagttatatcccctaacgccccataattacttgtctcattctctttttattcaaaat |
| | tttttgaccttggatgcccatggtaagagtgctgcagagcgcttttggcatccttctactgcca |
| | ctcaggctttggtcaaatggaaagatccacttactggatcttggcaaggcccagatccagtcct |
| | catatggggccgagggcatgtttgtgtttttccacaggatgcagaaggccctcggtggctgcca |
| | gaacgattggtgcgacatgtggaccctctacctgctgatgacattgatgascadmATGTTGTTT |
| | TATGTGTCCATCAGAAGGCAATCCTAGGGCGTGAGTGTCATTGA |
| 209 | Translation of ORF number 96 in reading frame 1 on the direct |
| | strand |
| | YCSPRPSVSMPDSDGQWCFPAPPRVRPPLCQWVSPQWXXICGQPNSRKSFGSSKILRKGARFDC |
| | CSFYKNADSRLDAVYSFPIALAHFTGTLDNHYPKHKLLQFFQHHDPIFPSIVSHAPLPAVPNVF |
| | TDGSNNGVAVYALNKKVTKRVQTPPASAQIVELRAVHKVLLDFASQSFNLFSDSHYVVRAVRNL |
| | ETVPFISTSNPVIQDLFLQIQQAIQLRCKKFYIGHIRAHSNLPGPLAAGNQIADSATQLIALTQ |
| | IEKAQKAHSLHHQNSQSLRLQYKILREAARQIIKQCPDCSHLQPVPHYGINPRGLRPNDLWQMD |
| | VTHIPEFGKLKYVHVSIDTFSGFVIASAQSGEATSHVIRHCLAAFAMIGTPKKLKTDNGSGYTS |
| | KKFALFCQQFLINHVTGIPYNPQRQGIVERTHGTLKVILQKIKKGELYPLTPHNYLSHSLFIQN |
| | FLTLDAHGKSAAERFWHPSTATQALVKWKDPLTGSWQGPDPVLIWGRGHVCVFPQDAEGPRWLP |
| | ERLVRHVDPLPADDIDXXXVVLCVHQKAILGRECH |
| 210 | ORF number 97 in reading frame 1 on the direct strand extends |
| | from base 107215 to base 107613 |
| | tgtgggacacagctccctagcccatgctggtattatgagccttgcgctccccctaattcctccc |
| | ccatcactggtcgttggtcgactgctcacagcagctcacggcagctcttgccggctgccagcca |
| | ctcatgctggttgccggccactcacgctgaccatcggccgctcctggcagcacacggcagcaca |
| | cagcagcccgcggcagctcacgccgacctctggctgctcgcagcccagctccagggagccgttg |
| | ttcacaatcttagctgtagagggtgcagctcactggcccatgtgggaatcgaaccggtgacctc |
| | gttgttaggcgcacggcgctccaaccacctgagccaccaggcggcccTGATTGTGTTTCTATAT |
| | ACTGtgtttccctga |
| 211 | Translation of ORF number 97 in reading frame 1 on the direct |
| | strand |
| | CGTQLPSPCWYYEPCAPPNSSPITGRWSTAHSSSRQLLPAASHSCWLPATHADHRPLLAAHGST |
| | QQPAAAHADLWLLAAQLQGAVVHNLSCRGCSSLAHVGIEPVTSLLGARRSNHLSHQAALIVFLY |
| | TVFP |
| 212 | ORF number 98 in reading frame 1 on the direct strand extends |
| | from base 107752 to base 107997 |
| | aataagaccgggttttatattaagttttgctccaaaagacgcattagagctgattgtccagcta |
| | ggtcttattttcggggaaacatggTAGAGAATCATACAGATTCTCTGCATATAAGGAATTTTGT |
| | AAAGGAGAAGGGTACTGAGCAGAGATTATATCTCTCAAATAACACTATTCTCTCTTCCTTTTTG |
| | ATTTTACAGTGGAGGAAAGGAGGACAAAGTACTAAAGTGAAAAGTAGATCTTGA |
| 213 | Translation of ORF number 98 in reading frame 1 on the direct |
| | strand |
| | NKTGFYIKFCSKRRIRADCPARSYFRGNMVENHTDSLHIRNFVKEKGTEQRLYLSNNTILSSFL |
| | ILQWRKGGQSTKVKSRS |
| 214 | ORF number 99 in reading frame 1 on the direct strand extends |
| | from base 113266 to base 113505 |
| | AGAGAACTGAGGTTGCTTGTCTTTATAGCTACTAGTGGCCTCAAAAGGCCAATACATCTGTCTC |
| | CATTTGTCCCTTGCTCAATACCCTCTGATTTACAAAGCCTTTCTTCTCTTAGGAAACGAATGGC |
| | AGAGAATGAACTGAGCCGGTCGGTGAATGAGTTTCTGTCCAAGCTGCAGGATGACCTCAAAGAG |
| | GCAATGAATACCATGATGTGCAGCCGATGCCAGGGAAAGCATAGGTAG |
| 215 | Translation of ORF number 99 in reading frame 1 on the direct |
| | strand |
| | RELRLLVFIATSGLKRPIHLSPFVPCSIPSDLQSLSSLRKRMAENELSRSVNEFLSKLQDDLKE |
| | AMNTMMCSRCQGKHR |
| 216 | ORF number 100 in reading frame 1 on the direct strand extends |
| | from base 113818 to base 114210 |
| | GGAGTTGTCCTTTTGTTGGGTTGTAGGAGGTTTGAAATGGACCGGGAACCTAAGAGTGCCAGAT |
| | ACTGTGCTGAGTGTAATAGGCTGCATCCCGCTGAAGAAGGAGACTTTTGGGCAGAGTCTAGCAT |
| | GTTGGGCCTGAAAATCACCTACTTTGCGCTGATGGATGGAAAGGTGTATGACATCACAGGTACA |
| | CTTCTGTCCTCTAGAATTCCAGACTCATGTATGCTCAAAACTGTTATGTATTGGCTAATTATTT |
| | CTCATGCTTGCAGAGTGGGCTGGATGCCAGCGTGTGGGAATCTCCCCAGATACCCACAGAGTCC |
| | CCTATCACATCTCATTTGGTTCTCGGATCCCAGGCACCAGTGGGCGACAGAGGTGGGTGATATT |
| | TTCCAATAA |
| 217 | Translation of ORF number 100 in reading frame 1 on the direct |
| | strand |
| | GVVLLLGCRRFEMDREPKSARYCAECNRLHPAEEGDFWAESSMLGLKITYFALMDGKVYDITGT |
| | LLSSRIPDSCMLKTVMYWLIISHACRVGWMPACGNLPRYPQSPLSHLIWFSDPRHQWATEVGDI |
| | FQ |
| 218 | ORF number 101 in reading frame 1 on the direct strand extends |
| | from base 114376 to base 114630 |
| | CTCTTAATTTCTTTTGCCTCATTATTCTTTTGTTTTCCACCCAGAGCCACCCCAGATGCCCCTC |
| | CTGCTGACCTTCAGGATTTCTTGAGCCGGATCTTTCAAGTACCCCCAGGACAGATGTCTAATGG |
| | GAACTTCTTTGCAGCTCCTCAGCCTGGCCCTGGGGGCACCGCAGCCTCCAAGCCTAACAGCACA |
| | GTACCCAAGGGAGAAGCCAAACCGAAGAGGCGGAAGAAAGTGAGGAGGCCCTTCCAACGTTGA |
| 219 | Translation of ORF number 101 in reading frame 1 on the direct |
| | strand |
| | LLISFASLFFCFPPRATPDAPPADLQDFLSRIFQVPPGQMSNGNFFAAPQPGPGGTAASKPNST |
| | VPKGEAKPKRRKKVRRPFQR |
| 220 | ORF number 102 in reading frame 1 on the direct strand extends |
| | from base 114631 to base 114945 |
| | CACCCCTTCTCTTCTCTCCTCAAATCAATGTCAGGGAGTCAAAAGGGCTGTGTACAGCACAGGA |
| | TGGAGTTTGATTTGTTTATTTTTAAATATTTAAAAAGGAAAATTTTAAGCTCAAATTGTTCACT |
| | CAGTACTTGTAGscadmgagaacaggtctggggtattttccccaggggtcatagatttacctgt |
| | actccaccaaaaaactgcaaaggcaataatttggaaaacagatacacctgtgtgaatagatcag |
| | tggccccttacacagaaaaagatatcggccgcccaggcgcttgtacaggagcagcttga |
| 221 | Translation of ORF number 102 in reading frame 1 on the direct |
| | strand |
| | HPFSSLLKSMSGSQKGCVQHRMEFDLFIFKYLKRKILSSNCSLSTCXXREQVWGIFPRGHRFTC |
| | TPPKNCKGNNLENRYTCVNRSVAPYTEKDIGRPGACTGAA |
| 222 | ORF number 103 in reading frame 1 on the direct strand extends |
| | from base 119038 to base 119274 |
| | gtgatagctccacgacctcgtgttacggagcttgagtgggctcgtaactgcgtttccggcactg |
| | tcttacggctaaacggcgatcaaaacttcggttttgccagggcgggggtttataccgccacgct |
| | taattgccacgatagtcttggtcccgcgaggggcacggccagccgagcatctgtgtgTTTTACT |
| | TGTGTGAAAGAAGGGCCGAGGATAAAGGGAAATGGGTCACGCTAA |
| 223 | Translation of ORF number 103 in reading frame 1 on the direct |
| | strand |
| | VIAPRPRVTELEWARNCVSGTVLRLNGDQNFGFARAGVYTATLNCHDSLGPARGTASRASVCFT |
| | CVKEGPRIKGNGSR |
| 224 | ORF number 104 in reading frame 1 on the direct strand extends |
| | from base 121210 to base 122190 |
| | caaagacggcaaacccttacagggaaactgggtgaggggccagccccaggccccgactcagcaa |
| | tgttatggggcactgcaggttcaggaacagacccaggagccgaaaaagaacgaacccctgctag |
| | gaagcatgtcacagacttattcagggccaccacaggcagcgcaggattggacttgtgttccacc |
| | tccgacatcatattaactcctgaaatgggaatgcaagttttgcccactggagtttttgggcccc |
| | tgccacctaaaacggtgggtttactgttaggaagaagcagctccgttataaagggaattcatgt |
| | ttctccagggattattgatgaggattttacaggagaaataaaaattatggctcattctcctctt |
| | aatatttctgccattcctgctggaacccgtattgcacaactgtttattttgcctcgtcttaata |
| | ttggaaaaaacaggcaaaatcaagagcgggggaaccaaggatttggctcttctgatgtatattg |
| | gattcaagaaataaaaaaggatcgacccgtattgttactcaaaataaatggaaaagattttcaa |
| | ggacttctggacactggagccgatgtctcgtgcatatctgctgaacattggccctccagttggc |
| | cgacgcgctttactaataccaatttacaaggcataggccaatcgcaatcccccctccaaagtag |
| | tgatcttttgtcttggcaagatccggagggtcatcaggggacgtttcagccatatattatccct |
| | ggtcttccagttaatttatggggaagagatgttatgagtaaaatgggagtttatctttacagtc |
| | ctagttcacaagtaactcaacagatgtttgatcaaggctttctccctggtcagggcttaggctc |
| | ggtgggacaagggcgccgagagcctatttcaactaatcctaacttacagagaacaggtctgggg |
| | tattttccccaggggtcatag |
| 225 | Translation of ORF number 104 in reading frame 1 on the direct |
| | strand |
| | QRRQTLTGKLGEGPAPGPDSAMLWGTAGSGTDPGAEKERTPARKHVTDLFRATTGSAGLDLCST |
| | SDIILTPEMGMQVLPTGVFGPLPPKTVGLLLGRSSSVIKGIHVSPGIIDEDFTGEIKIMAHSPL |
| | NISAIPAGTRIAQLFILPRLNIGKNRQNQERGNQGFGSSDVYWIQEIKKDRPVLLLKINGKDFQ |
| | GLLDTGADVSCISAEHWPSSWPTRFTNTNLQGIGQSQSPLQSSDLLSWQDPEGHQGTFQPYIIP |
| | GLPVNLWGRDVMSKMGVYLYSPSSQVTQQMFDQGFLPGQGLGSVGQGRREPISTNPNLQRTGLG |
| | YFPQGS |
| 226 | ORF number 105 in reading frame 1 on the direct strand extends |
| | from base 122728 to base 123048 |
| | ctttggatgcctatgttaagagtgcagctgaacgtttctggcatccttctgccgtccctgaggc |
| | tttggtcagaaagaaggatccacttactggatcatggcaaggcccagacccagtcctcatatgg |
| | ggccgagggcatgtttgtgtttttccacaggatgcagatagtcctcggtggctgccagaacgat |
| | tggtgcgacatgtggaccctctacctgctgatgacattgatgaccctcagcaatgcagaagaag |
| | accagacgtattgggcctacgtacctgatccacctattctccaccctgctgtatscadmatgta |
| | a |
| 227 | Translation of ORF number 105 in reading frame 1 on the direct |
| | strand |
| | LWMPMLRVQLNVSGILLPSLRLWSERRIHLLDHGKAQTQSSYGAEGMFVFFHRMQIVLGGCQND |
| | WCDMWTLYLLMTLMTLSNAEEDQTYWAYVPDPPILHPAVXXM |
| 228 | ORF number 106 in reading frame 1 on the direct strand extends |
| | from base 123565 to base 123798 |
| | ggcgtgagtgtcactgacataatctggaatctcaggaccatcccatacagcagggtggagaata |
| | ggtggatcaggtacgtaggcccaatacgtctggtcttcttctgcattgctgagggtcatcaatg |
| | tcatcagcaggtagagggtccacatgtcgcaccaatcgttctggcagccaccgaggactatctg |
| | catcctgtggaaaaacacaaacatgccctcggccccatatga |
| 229 | Translation of ORF number 106 in reading frame 1 on the direct |
| | strand |
| | GVSVTDIIWNLRTIPYSRVENRWIRYVGPIRLVFFCIAEGHQCHQQVEGPHVAPIVLAATEDYL |
| | HPVEKHKHALGPI |
| 230 | ORF number 107 in reading frame 1 on the direct strand extends |
| | from base 125896 to base 126126 |
| | GCGGTGAGGACGTGTGCGCCCTTCCTCCTTCCTCTTTCTCGACTCCATCTTCGCGGTAGCGGTA |
| | GCGGCCGCAGTTCAGGTAAGATTTGGGCCACGGCTGGATCCGGACGACTTAATAGGTTAGCCGC |
| | GAGGTCTGACGGCTTGGGAAAAATAGAGGAAGAGGGGCTGCTCTGTGGGCCGGGTTCTTGTCAC |
| | CACCCGACCTCCCTGGCTGGCCTGGCCTTAGGCACGTGA |
| 231 | Translation of ORF number 107 in reading frame 1 on the direct |
| | strand |
| | AVRTCAPFLLPLSRLHLRGSGSGRSSGKIWATAGSGRLNRLAARSDGLGKIEEEGLLCGPGSCH |
| | HPTSLAGLALGT |
| 232 | ORF number 108 in reading frame 1 on the direct strand extends |
| | from base 126127 to base 126387 |
| | GACCCGCGATCGTCCCCGGCCCGCCACCCACTCCCCGACTCCCTTACTCCCAGAGCATTTCTTC |
| | TCTTACAAGCATTTCTTTCCTCAGTCGCCGACATGCAGCTCTTTGTTCGCGCCCAAGATCTACA |
| | CACCCTCGAGGTGACCGGCCAGGAGACTGTCTCCCAGATCAAGGTAAGGCTGCGTGGTGCTCCT |
| | GGTCTGCATCCTCTTGTGTTCTTTAACCTCGCTCCCCACGGGAGCGCTGAGCCTCACTTTCCCC |
| | TGTAG |
| 233 | Translation of ORF number 108 in reading frame 1 on the direct |
| | strand |
| | DPRSSPARHPLPDSLTPRAFLLLQAFLSSVADMQLFVRAQDLHTLEVTGQETVSQIKVRLRGAP |
| | GLHPLVFFNLAPHGSAEPHFPL |
| 234 | ORF number 109 in reading frame 1 on the direct strand extends |
| | from base 126961 to base 127260 |
| | AGTCCATGGTTCCTTGGCCCGTGCTGGGAAAGTAAGAGGTCAGACTCCCAAGGTAAGAGAGTAT |
| | TAGTGGTGCCCTTTGGACTTTTGTTTTCCTGTCACCTTCCTCATGAAATGAGCCTGAGGGAAGG |
| | CACGGAAGAGATGAACCAGGGTCTGATTAGCCCTCCTTTTTCCCAGGTGGCCAAACaggagaag |
| | aagaagaagaagaCTGGCCGAGCCAAGCGGCGGATGCAGTACAACCGGCGTTTTGTCAATGTTG |
| | TGCCCACCTTTGGCAAGAAGAAGGGCCCCAATGCCAACTCTTAA |
| 235 | Translation of ORF number 109 in reading frame 1 on the direct |
| | strand |
| | SPWFLGPCWESKRSDSQGKRVLVVPFGLLFSCHLPHEMSLREGTEEMNQGLISPPFSQVAKQEK |
| | KKKKTGRAKRRMQYNRRFVNVVPTFGKKKGPNANS |
| 236 | ORF number 110 in reading frame 1 on the direct strand extends |
| | from base 129976 to base 130284 |
| | ccttggatgcccatggtaagagtgctgcagagcgcttttggcatccttccactgccactcaggc |
| | tttgttcaaatggaaagacccacttacaggctcttggcaaggcccagatccagtcctcatatgg |
| | ggccgagggcatgtttgtgtttttccacaggatgcagaaggccctcggtggttgccagaacgat |
| | tggtgcgacatgtggaccctctacctgctgatgacattgatgascadmTTACAAAACTTTCCAA |
| | ATGTTGTTTTATGTGTCCATCAGAAggcaatcctagggcgtgagtgtcattga |
| 237 | Translation of ORF number 110 in reading frame 1 on the direct |
| | strand |
| | PWMPMVRVLQSAFGILPLPLRLCSNGKTHLQALGKAQIQSSYGAEGMFVFFHRMQKALGGCQND |
| | WCDMWTLYLLMTLMXXLQNFPNVVLCVHQKAILGRECH |
| 238 | ORF number 111 in reading frame 1 on the direct strand extends |
| | from base 130801 to base 131133 |
| | aggggaatgggacttaattggggaacagtgtgtacttccaggacattttccaagtcaagttgtc |
| | ctttcagtcttagttgtggagggcactgttcagccccaggtccagttgccgttgttagttgcag |
| | ggggtggagcccagcaccccttgcgggagttgaaccagcaagcttgtggttgagagcccactgg |
| | cccatgtgggctctggaaccggcagccttcaatgttaggagcacagagctccaaccgcctgagc |
| | cactgggccggcccACCCCCCCTTTTTTTTTTTTTAAGAAAAAGTATTTTTTTCTCTCAAAAGC |
| | TTCCTTATATTAG |
| 239 | Translation of ORF number 111 in reading frame 1 on the direct |
| | strand |
| | RGMGLNWGTVCTSRTFSKSSCPFSLSCGGHCSAPGPVAVVSCRGWSPAPLAGVEPASLWLRAHW |
| | PMWALEPAAFNVRSTELQPPEPLGRPTPPFFFFKKKYFFLSKASLY |
| 240 | ORF number 112 in reading frame 1 on the direct strand extends |
| | from base 131335 to base 131946 |
| | GGGAGAATGAATGAATTAGCCTTTGAAGCTGATGTGTCTGATTTGGTTCTTTTCCTCTCAGGTG |
| | AAAAGCTCCGGGTCTTAGGCTACAATCACAATGGCGAATGGTGTGAAGCCCAAACCAAAAATGG |
| | CCAAGGGTGGGTTCCCAGCAACTACATCACGCCCGTCAACAGCCTGGAGAAACATTCCTGGTAC |
| | CACGGGCCCGTGTCCCGCAATGCTGCCGAGTACCTGCTGAGCAGTGGGATCAACGGCAGCTTCC |
| | TGGTGCGGGAGAGTGAGAGCAGCCCCGGGCAGAGGTCCATCTCGCTGAGATACGAAGGGAGGGT |
| | GTACCACTACAGGATCAACACAGCTTCGGACGGCAAGGTgggcggggcggggcgccgggggcgg |
| | ggcCTGAGTCTTGGGCCAGAACTCAGAGATCCCTCTGCTGGGTGGATAATGTTTTTACGACAAT |
| | ACTCGAGAAGTGGTTGGCAGACACTTTCATGTAAACAGCAGGCGTCATTCATTAGCCTCATCGA |
| | TGATCCCCTGTGGAGGACTGATCATGTGACATTACAAGTCCACGGGCTGGGCTGGTTCTCTGGT |
| | TGTCCTGCTGGACGTTTGTTGTTAACAGTTTCATAA |
| 241 | Translation of ORF number 112 in reading frame 1 on the direct |
| | strand |
| | GRMNELAFEADVSDLVLFLSGEKLRVLGYNHNGEWCEAQTKNGQGWVPSNYITPVNSLEKHSWY |
| | HGPVSRNAAEYLLSSGINGSFLVRESESSPGQRSISLRYEGRVYHYRINTASDGKVGGAGRRGR |
| | GLSLGPELRDPSAGWIMFLRQYSRSGWQTLSCKQQASFISLIDDPLWRTDHVTLQVHGLGWFSG |
| | CPAGRLLLTVS |
| 242 | ORF number 113 in reading frame 1 on the direct strand extends |
| | from base 132532 to base 132804 |
| | GGGTGTAGCCAGATGGATTGTCGGTGTGGCTCCAGATGGTGTATATATTTTTTAGTAATATGTA |
| | ATGTATGCACACGGTTTTTAAAAAAATCAATTACAGTGAAAGGTAATTTCGTTTCTAGTTTAGT |
| | TCCCTGCCCAGAAGCAATCACTGTAACCACCTTCTTCCAGAGTAACACGGTGTTATATACACGG |
| | TGTATATActgtgtttccctgaaaataagacctaaccggacagtaagccctagcatgatttttc |
| | aggatgacgtcccctga |
| 243 | Translation of ORF number 113 in reading frame 1 on the direct |
| | strand |
| | GCSQMDCRCGSRWCIYFLVICNVCTRFLKKSITVKGNFVSSLVPCPEAITVTTFFQSNTVLYTR |
| | CIYCVSLKIRPNRTVSPSMIFQDDVP |
| 244 | ORF number 114 in reading frame 1 on the direct strand extends |
| | from base 134401 to base 134862 |
| | CTTGTAGAATTTGAGAGTCAGCCAATGAGGAAGCCGACCCCTCTGTCTAAAAGCTGGTGTGTGC |
| | TGGGGCTCCTTTCACTCCGGGTGGAACTCAGGGAGTTCATTTGCTCAAGCACTGTCCACCCCCG |
| | GGCAGCTCGTCAGACAGTTCTGGGCTTCTCGccctcctccctccctccctccAGCTGTCTGAGC |
| | ACCTGGAGCCTCCTGGGCCTACAGGGTCATCGGGCAGACCCTCTGCAGAGGCTCCTGCCTGTGT |
| | TGGGTGGGAGCACATTCCAAAAGGAGTGGAACAGTGTCTGCATGGGGAGGTACTCCAGTGATGC |
| | AGGCGACAGCCTGGCACTGAGGAGCTGCTCCAAGCGGAGCTTTGAGGGGATCCTTTTAGGATTT |
| | CTAAGGGGAACATTTAAGGCTGGTAGGAGGGACAGGCTGGGGTTGAAGAAATTTAGTTCTTATT |
| | TTCAAATGAGCTGA |
| 245 | Translation of ORF number 114 in reading frame 1 on the direct |
| | strand |
| | LVEFESQPMRKPTPLSKSWCVLGLLSLRVELREFICSSTVHPRAARQTVLGFSPSSLPPSSCLS |
| | TWSLLGLQGHRADPLQRLLPVLGGSTFQKEWNSVCMGRYSSDAGDSLALRSCSKRSFEGILLGF |
| | LRGTFKAGRRDRLGLKKFSSYFQMS |
| 246 | ORF number 115 in reading frame 1 on the direct strand extends |
| | from base 136801 to base 137037 |
| | GGTAGTAGGATCGCTACGAAAAGACTGTCAGTTATAAAACCTCTGAGCCAGAGTTTGCTATTGG |
| | CTTGCCTGACTTTTAACTGTCCATGTGTGTCATCTCCCCAGAACagagagagagagagagagag |
| | agagagagagagaaagagagagagaATCTCCTTGTTAATGAATCCTGCTTACCTTCTTGAGGGT |
| | TATAGAAGGTATCAACTTGTATATGTTGTTATTTCTCTCTTTTAA |
| 247 | Translation of ORF number 115 in reading frame 1 on the direct |
| | strand |
| | GSRIATKRLSVIKPLSQSLLLACLTFNCPCVSSPQNRERERERERERKRERISLLMNPAYLLEG |
| | YRRYQLVYVVISLF |
| 248 | ORF number 116 in reading frame 1 on the direct strand extends |
| | from base 137737 to base 138054 |
| | AAAGAGAAGAAAAATGATAGCTGTCCCCATCCACATTGCGCCCTCTGTCGTGTGCTCCTTTCCC |
| | TTCTCTCGTCTCAGTTGGTCCGGACGAGAACTCCTTGTGGAGGGGCTTCCTGCACAGGTGCTCA |
| | CCACTGTCCATCTCACAGGAGACTCATGTGCGTGTGTCTGAAAACCCTCTTCCTGCCTTCCCGG |
| | CCATGGAAAAACCTGGATGGCCTTGGGCAGCCCTCCAGCCCCTGCTCTGTTCCTGGAGAGCACT |
| | GGCCAAGGAACCACGGGGTGTATTACTGGGTCACGGGGTGTACTGCAGGTCTTGATCTATGA |
| 249 | Translation of ORF number 116 in reading frame 1 on the direct |
| | strand |
| | KEKKNDSCPHPHCALCRVLLSLLSSQLVRTRTPCGGASCTGAHHCPSHRRLMCVCLKTLFLPSR |
| | PWKNLDGLGQPSSPCSVPGEHWPRNHGVYYWVTGCTAGLDL |
| 250 | ORF number 117 in reading frame 1 on the direct strand extends |
| | from base 138724 to base 139011 |
| | GGCTTCGCTGTGCATCGCGTTTCGTTAGCAGCAAAGCTGGTTCGTTGGCGTTGTTTGCGTTGGT |
| | GTCTGCTCTGTGGCCTGAAGGCTGTCCCTGTTTTCCTCAGCTCTACGTCTCCTCAGAGAGCCGC |
| | TTCAACACTTTGGCCGAGTTGGTTCATCATCACTCCACTGTGGCAGACGGGCTCATCACCACTC |
| | TCCACTATCCAGCCCCCAAGCGCAACAAGCCCACCGTCTACGGCGTGTCTCCCAACTATGACAA |
| | GTGGGAGATGGAGCGCACGGACATCACCATGA |
| 251 | Translation of ORF number 117 in reading frame 1 on the direct |
| | strand |
| | GFAVHRVSLAAKLVRWRCLRWCLLCGLKAVPVFLSSTSPQRAASTLWPSWFIITPLWQTGSSPL |
| | STIQPPSATSPPSTACLPTMTSGRWSARTSP |
| 252 | ORF number 118 in reading frame 1 on the direct strand extends |
| | from base 139498 to base 139740 |
| | CCAAAAAGCGCTCAGCTCTTCTGTGGATTTTTGTTGGCAGATTTGAAATGCAAGTGCTGCTTAG |
| | TTCCTAGCAGGTTCCTGTTCTTTGTATTGTGTGTCCAGACTTCTGGAATGAAGCAAACATTAAG |
| | GCTTCTTACTAACTCAGATCAGCCCTTCCCCCCTTCTTTCTTGTTATCTGTGACTTGCACCCTC |
| | GCCACTAATGCACAGTGTTTGTGGTTTCCAGGCGCTTTGTTTTTCTTTTGA |
| 253 | Translation of ORF number 118 in reading frame 1 on the direct |
| | strand |
| | PKSAQLFCGFLLADLKCKCCLVPSRFLFFVLCVQTSGMKQTLRLLTNSDQPFPPSFLLSVTCTL |
| | ATNAQCLWFPGALFFF |
| 254 | ORF number 119 in reading frame 1 on the direct strand extends |
| | from base 142240 to base 142551 |
| | AAATCACTTCTTCCCCTCTCCCCTTCTCCGCCATTTGCCCCCCTCAGAGTCTATAGCTGTGATC |
| | TACCTTGCTCTTCAAGACTCCTTGGGAAACCCGTGCAGCTCCAGCTCCAGCTTTCGTTTGCTCA |
| | GCGGTTCTCACCAAGCACCTCTTCACCTCTCCATGCCAGTCCTCACTGGGCACCTGAGTCTCGG |
| | TCCCCTCCTGCCTCCCTGTCCTGCCTGTTTTGCCTTGCTGGCCCCGCAAAGGGCAGTGCCAGCT |
| | CCTCCTTAGCCAGCAGGGGGAGCAAGGCCGGACTTTTAACCGCGACTCCATATTGA |
| 255 | Translation of ORF number 119 in reading frame 1 on the direct |
| | strand |
| | KSLLPLSPSPPFAPLRVYSCDLPCSSRLLGKPVQLQLQLSFAQRFSPSTSSPLHASPHWAPESR |
| | SPPASLSCLFCLAGPAKGSASSSLASRGSKAGLLTATPY |
| 256 | ORF number 120 in reading frame 1 on the direct strand extends |
| | from base 143080 to base 143724 |
| | AAGCACATGGCAGCATGCTGTGGACACTGGTCTGTAGCCTACTGTCCACTGACTGTATCCGCAC |
| | AGCTGTTCCTTGTCGGTACACATAAGGTCGCCTTGTTTTTATGTGGTGGATGTCAGCATGTAGC |
| | AGCCCTCTGTGGGCATTTGCGTTCTTCCCAGTGCGTGGCTGTTACAGAAGTGCTGCAGGGATTC |
| | TCCTTGTTTGCACACAGGGGACAGTGTCCTGGAGGGCCAGCACTCAGAGGGGAACGACTGCGTC |
| | AGGGGCCGTGTGTGTTTGTCGTCTTCCTCACACTCCCAAAGCCTCCCAAGGAGCTCGTACCTGT |
| | CTGCGCTCTGCCGCGCGTGTTGGGGGAGTGCCTGCTTCCCGTCCCTGCACTGACACAGTGTGCT |
| | TTGCTTTGGGGTTTATTTTTGTCATTTTCCCCCAGGAAATTTATTGGCAAGCTCAGAAACGAGC |
| | AGAGAAGGAAAGGTTCCGTGACAGCACTGACACTAGACCGGCCCACGCAGTGGCCATGTGACTA |
| | CGCGGGGGGTGTGCACCAGGGAGAGGCCACCATTGCCGTGTGGCACTTGCTGTTACACTGGGTT |
| | CTCTTCTGGCTGTGCAGCGAGACCCAGCTGCCGTGTTTGGGGACCAGACTTCTGGGGGCTCCTC |
| | TGTGA |
| 257 | Translation of ORF number 120 in reading frame 1 on the direct |
| | strand |
| | KHMAACCGHWSVAYCPLTVSAQLFLVGTHKVALFLCGGCQHVAALCGHLRSSQCVAVTEVLQGF |
| | SLFAHRGQCPGGPALRGERLRQGPCVFVVFLTLPKPPKELVPVCALPRVLGECLLPVPALTQCA |
| | LLWGLFLSFSPRKFIGKLRNEQRRKGSVTALTLDRPTQWPCDYAGGVHQGEATIAVWHLLLHWV |
| | LFWLCSETQLPCLGTRLLGAPL |
| 258 | ORF number 121 in reading frame 1 on the direct strand extends |
| | from base 145531 to base 145887 |
| | CTTGTCCTCTGGAAGTCTTCCCTCAGATCCGCGGCCAGCGGCGAATGCGGCAATCCTGGGCAGT |
| | TGTGCCGTAAGCACACCTTAGAGCCTGGTCGCCCCGAGGGGCAGGTCCCACATTTCAATAAACT |
| | CGATAAAGCTTTCTTCTTGGGGGAGGCTAGTTTTCAAGACGTTCACTCCCCATCTCCCATACAG |
| | TCTTTCTCTTCAGACAATTCAAACTCCCTGTGGAAACTTGAAGGGTGGGCTCTTGCCTCCCTGG |
| | TGGGCCTTTGTAGCCAAGTTCTCACAGCAAACAGATCGTGTCATTTACCGCCACCCGCTTCCTG |
| | TTTTGAGGGTCAGTTCAGAGGACAGTGGGTCCTTTAA |
| 259 | Translation of ORF number 121 in reading frame 1 on the direct |
| | strand |
| | LVLWKSSLRSAASGECGNPGQLCRKHTLEPGRPEGQVPHENKLDKAFFLGEASFQDVHSPSPIQ |
| | SFSSDNSNSLWKLEGWALASLVGLCSQVLTANRSCHLPPPASCFEGQFRGQWVL |
| 260 | ORF number 122 in reading frame 1 on the direct strand extends |
| | from base 146674 to base 146928 |
| | TTTCACTACCTTTTTTTCCTACAGGAGGACACCATGGAGGTGGAAGAGTTTTTGAAGGAAGCTG |
| | CGGTAATGAAAGAGATCAAGCACCCTAACCTAGTACAGTTACTTGGTGAGTGCGAGGAGCTCGG |
| | AAGGGGGGGCCTTTGCATTAAACCCGCTGGGGTGATCCAGGTGCTGTCAAAGAGGAGATGGCTG |
| | CCTCGCTACATGAATTCTTCTCATTTGGACATCTGTTCTCTACTAACATTCAGCCCTCGGTAA |
| 261 | Translation of ORF number 122 in reading frame 1 on the direct |
| | strand |
| | FHYLFFLQEDTMEVEEFLKEAAVMKEIKHPNLVQLLGECEELGRGGLCIKPAGVIQVLSKRRWL |
| | PRYMNSSHLDICSLLTFSPR |
| 262 | ORF number 123 in reading frame 1 on the direct strand extends |
| | from base 147094 to base 147399 |
| | TTTAGGCCATTTGATGTGTGCCTGGCCTTTGCTTCTGAACTCGGTGGCAGCCTCTTCCTGTTTA |
| | AGTTCATTGGCTTGAGAGGAAGAAAAGAGCAGGCCATGTACCACCCCCTGTCTCCCCCCCCAGA |
| | AACATCATCTCAAGTCACAGGTGCTTGGAACCGTCTTAGCACTGAGTCCAGGGCTTGGGGGCAG |
| | AGTCAGATCCATTTCAGAAGCCTTTTCCTTGAGGTCCAGTCCTTTCTGATGCCTGTGCTGTGTC |
| | TCGTTGGCAGGGGTCTGCACCCGGGAGCCCCCGTTCTATATAATCACTGA |
| 263 | Translation of ORF number 123 in reading frame 1 on the direct |
| | strand |
| | FRPFDVCLAFASELGGSLFLFKFIGLRGRKEQAMYHPLSPPPETSSQVTGAWNRLSTESRAWGQ |
| | SQIHFRSLFLEVQSFLMPVLCLVGRGLHPGAPVLYNH |
| 264 | ORF number 124 in reading frame 1 on the direct strand extends |
| | from base 147445 to base 147708 |
| | CCGGCAGGAGGTGAACGCTGTGGTGCTGCTGTACATGGCCACGCAGATCTCGTCAGCCATGGAG |
| | TACCTGGAGAAGAAAAACTTCATCCACAGGTAGGAGCCTGCCGAGGCCGCCTCCCCACAGGGCC |
| | CCGGCACCCTTCTGTAAAAGGCCCCACCTTGAGGGGTGACCGCTCGGCCTCTCCCTTCAGTGCT |
| | GGCAACATGTTAGGTCTGAGACAAGAGCGCAGCGGTGGGTTCCGACGTGGCCAGCTCTGGGTGT |
| | GTGTCTAG |
| 265 | Translation of ORF number 124 in reading frame 1 on the direct |
| | strand |
| | PAGGERCGAAVHGHADLVSHGVPGEEKLHPQVGACRGRLPTGPRHPSVKGPTLRGDRSASPESA |
| | GNMLGLRQERSGGFRRGQLWVCV |
| 266 | ORF number 125 in reading frame 1 on the direct strand extends |
| | from base 147796 to base 148275 |
| | GGGGCATACTCAGTGTTTCATACAAGGAGTCGAGTGCTCCTTGTTCCGCCGAGCCCAGCCGGCG |
| | GGCGCCGTAGTGACCTCTTCCCCGGAGCGGGTGGCCCTGCCCTGACACACGGCAAGAGCGGCCA |
| | GTGCATGGGTTTCGGTTTTGTGCTGCGTGTTTTTTTTCTCCCTTCTCTTTATTATCATTTCATT |
| | CTCCACTTAACTTGCTGTCACCGGCCTCGGCAATGTTTCCACAATTGGCAGAATTGTGTAGATG |
| | CGGCTCTAAGTGAAGTGTCTTTGCTGTTTCAAAGCCCGGAGTGTTGTGACCTTCAGGTGCGCCA |
| | CAATTATCCTGGTCTTCACATTCTTTGCTGGTGGAAATGGCTTCCTAGCAGAGTGACAGCCTAT |
| | CCAGGGCAGAGCCTGTGGGCTTTGCCAGAGTCGTTCATACAAGACATTCTCTCTGCCACCACTG |
| | TGACCTTTCCTGTCCAATTATCTCGACTATGA |
| 267 | Translation of ORF number 125 in reading frame 1 on the direct |
| | strand |
| | GAYSVFHTRSRVLLVPPSPAGGRRSDLFPGAGGPALTHGKSGQCMGFGFVLRVFFLPSLYYHFI |
| | LHLTCCHRPRQCFHNWQNCVDAALSEVSLLFQSPECCDLQVRHNYPGLHILCWWKWLPSRVTAY |
| | PGQSLWALPESFIQDILSATTVTFPVQLSRL |
| 268 | ORF number 126 in reading frame 1 on the direct strand extends |
| | from base 153391 to base 153885 |
| | AAAAAAAAAAGGAAACCAACATACCAACATGACAGCATTACTGATGGCTGCTGCTTTTtgtgtt |
| | gtttttgtgtgtgtgtgtgtatgtgGTTCTTAGAAGTGGAAAAGGAACTGGGGAAAAAAGGCAT |
| | GCGAGGGGTTGCAAGCACTCTGCTGCAGGCCCCAGAGCTGCCCACCAAGACAAGAACCTCCAGG |
| | AGAGCTGTGGAACACAAAGACCCCACCGACGTGCCCGAGACACCCCACTCCAAGGGCCCGGGAG |
| | AGCCTGGTATGTCTGCACCCCACCCCCACTGCAGGCTCAGGGTCAGTGCCCTTAGGGCCAGGGT |
| | GGCAGACGGGGAGCAGTGCGCGCAGCCTGCACAGAAAGGCAGGCAAACTCCCATTAGTTGTCCA |
| | GCGGTGGAGAAGGTTCTTCTCTCCCTGCAGCATCCCACCCTCCCTCTGGGAATCGTTAGGGGCC |
| | ATTGGCTTCAGCAGGTAGTTCAGTCTGATGGGCAGAGGTGCTTCTGA |
| 269 | Translation of ORF number 126 in reading frame 1 on the direct |
| | strand |
| | KKKRKPTYQHDSITDGCCFLCCFCVCVCMWFLEVEKELGKKGMRGVASTLLQAPELPTKTRTSR |
| | RAVEHKDPTDVPETPHSKGPGEPGMSAPHPHCRLRVSALRARVADGEQCAQPAQKGRQTPISCP |
| | AVEKVLLSLQHPTLPLGIVRGHWLQQVVQSDGQRCF |
| 270 | ORF number 127 in reading frame 1 on the direct strand extends |
| | from base 155347 to base 155637 |
| | AAACTGGAAAAGGTCACCCCTTCTTGTTTCCCAAGCATAATGGCCCAGTGTCACTGCACTCTGT |
| | GGGATGTGTCCCGTTCCCTCCAGGTCACACCCTGTAGAAACCACCAGTTGGCTGGTCTGAGAGG |
| | CACAGGTTATGACCCTTTGCTCGGCCGTGTCATAGTTTTTACTCACAAGATAGTGAGGGGACTC |
| | TGCAGATATAAAGGAAACCAGTGCAGGGGTGGGGGAGACGGGGACGTCCCGGCTTTTTGTTCTG |
| | CTGTCTTCAAGGAGAGAGACCTAAGCTCTTCCtaa Translation of |
| 271 | ORF number 127 in reading frame 1 on the direct strand |
| | KLEKVTPSCFPSIMAQCHCTLWDVSRSLQVTPCRNHQLAGLRGTGYDPLLGRVIVFTHKIVRGL |
| | CRYKGNQCRGGGDGDVPAFCSAVFKERDLSSS |
| 272 | ORF number 128 in reading frame 1 on the direct strand extends |
| | from base 156277 to base 156714 |
| | GTGCGGCGGGGGGCGGCCGCGGCCAGTGGGGGGGGGCGCTGGAGTTGGGGCGGCAGGGCCGAGC |
| | GGCCCGGGGCGGGGAGTCGCTGTCCTCGCCGAGCGCGCGGGCGCACGGGGGCGCAGGTGAGCCG |
| | CGGGCGGGGCGCTGCGGCTGGGGGCTGGGGGCGGCAGGGCGGCTTCGTGTGCCACTCGGCCTCG |
| | GCAGGCCAGCTCTTCGAGCTCCGTGTCCCTGGCTCTGTCCTCCTTGGGACCCCACAAGTGCCCT |
| | CAGGAAGGCTGTGGGGTTCCCCTGCGCCGAGGCCCACCCGTGGCCATGCGCTAGGAGGTGTCTC |
| | CCACCCGCCGGAGTCCCAAGGACCCCTCCCAAGAGCTCGGGCACCCTGCGGCCATCACACCCAA |
| | CAGGCGAGTCGGGGTGTAGGAAGTCCACTGCTCACAAGGGCACCCCCTCATTAA |
| 273 | Translation of ORF number 128 in reading frame 1 on the direct |
| | strand |
| | VRRGAAAASGGGRWSWGGRAERPGAGSRCPRRARGRTGAQVSRGRGAAAGGWGRQGGFVCHSAS |
| | AGQLFELRVPGSVLLGTPQVPSGRLWGSPAPRPTRGHALGGVSHPPESQGPLPRARAPCGHHTQ |
| | QASRGVGSPLLTRAPPH |
| 274 | ORF number 129 in reading frame 1 on the direct strand extends |
| | from base 156715 to base 156966 |
| | ACATCAGAAATTGGAGACACCCCGGATGGATGGGGGCCTTGGCCCCAAACCCTTTTTCTGTCCC |
| | ACCTGTTTCCGTGCCCCTACACCTCCTGTGGGTCTTTTCTTGTCTGTGAGTCTGTGCTTACCAG |
| | GGGGAACCCTGGGCCCACAGGGCCTCCTCACTCACCTGCCTTGTTTTCTCAGAACTTCTCATGG |
| | CTGCAGGCCCCATGGGTTTCCCTTAGTTTAACTTatgtgggtcttctccttggagcgtaa |
| 275 | Translation of ORF number 129 in reading frame 1 on the direct |
| | strand |
| | TSEIGDTPDGWGPWPQTLFLSHLFPCPYTSCGSFLVCESVLTRGNPGPTGPPHSPALFSQNFSW |
| | LQAPWVSLSLTYVGLLLGA |
| 276 | ORF number 130 in reading frame 1 on the direct strand extends |
| | from base 157057 to base 157377 |
| | atacttgtcgaatgCACCGACATGCCCAGTGGGGCCTGGAACCTGTCGTCGGTTGGCACTGGCC |
| | TGCCTGGGCACGCTGCTGTGTGCTCCACCGTGGCAGGACCTGTTCCCTTAGGGAGGGGGACTGG |
| | TGACCTCAGCCTGGGCGCCTCCAGTTCGGGCTTTCTGCCTACTCAGCAACTTCTAATTTGGGTG |
| | CGTGGTTGGGAGATGCTCTCAGCTGTCAGTCCTGCCCTTGGGGGGCCAGCTTCCTGCCTCTCAC |
| | AGCCATTAAGTGCAGCTGGACGCAGGACCCCTGTCCCACTCCTGGGCTGCAGGAGCCACAGGTG |
| | A |
| 277 | Translation of ORF number 130 in reading frame 1 on the direct |
| | strand |
| | ILVECTDMPSGAWNLSSVGTGLPGHAAVCSTVAGPVPLGRGTGDLSLGASSSGFLPTQQLLIWV |
| | RGWEMLSAVSPALGGPASCLSQPLSAAGRRTPVPLLGCRSHR |
| 278 | ORF number 131 in reading frame 1 on the direct strand extends |
| | from base 157717 to base 158037 |
| | CACAAGCTTTTCTGCCTGTTGCACCGAGGGGGACCCTCGTCCTCGGACCTGAGGGCACAAGAGG |
| | TGCAGGGAGGGGCTCGTGGTGCACATACTGCGTCCCAGGAGGGGTGGGGGTCCCTAAGCAGTGT |
| | CCTCGCGCAGGACTCCTACCGGAAGCAAGTGGTCATCGATGGGGAGACGTGTCTGCTGGACATC |
| | CTGGACACGGCGGGCCAGGAGGAGTACAGCGCCATGCGGGACCAGTACATGCGCACCGGCGAGG |
| | GTTTCCTCTGCGTGTTTGCCATCAACAACACCAAGTCCTTTGAAGACATCCACCAGTACCGGTG |
| | A |
| 279 | Translation of ORF number 131 in reading frame 1 on the direct |
| | strand |
| | HKLFCLLHRGGPSSSDLRAQEVQGGARGAHTASQEGWGSLSSVLAQDSYRKQVVIDGETCLLDI |
| | LDTAGQEEYSAMRDQYMRTGEGFLCVFAINNTKSFEDIHQYR |
| 280 | ORF number 132 in reading frame 1 on the direct strand extends |
| | from base 158281 to base 158505 |
| | GCTGGCTCCCTGCCCACCTGTAGCCAGGGCCCCGCCCGCCCCGCCAGGGAGCCGTGCTCACCGC |
| | CCCTCTCCCTCGACACAGGGCAGCCGCTCTGGCTCCAGCTCCGGGACCCCGGGACCCAGCGGCC |
| | CCTCGCGCTGTscadmCGGAGCCCATGCGCCGGAGGAGCTgcgcgccccggcccccgcccccgc |
| | ccgacccggcccggGGGGCTGTCGCTCCAGTGA |
| 281 | Translation of ORF number 132 in reading frame 1 on the direct |
| | strand |
| | AGSLPTCSQGPARPAREPCSPPLSLDTGQPLWLQLRDPGTQRPLALXXRSPCAGGAARPGPRPR |
| | PTRPGGLSLQ |
| 282 | ORF number 133 in reading frame 1 on the direct strand extends |
| | from base 158506 to base 159063 |
| | GCGGTGAGTGCGGCGGGGGGCGGCCGCGGCCAGTGGGGGGGGGCGCTGGAGTTGGGGCGGCAGG |
| | GCCGAGCGGCCCGGGGCGGGGAGTCGCTGTCCTCGCCGAGCGCGCGGGCGCACGGGGGCGCAGG |
| | TGAGCCGCGGGCGGGGCGCTGCGGCTGGGGGCTGGGGGCGGCAGGGCGGCTTCGTGTGCCACTC |
| | GGCCTCGGCAGGCCAGCTCTTCGAGCTCCGTGTCCCTGGCTCTGTCCTCCTTGGGACCCCACAA |
| | GTGCCCTCAGGAAGGCTGTGGGGTTCCCCTGCGCCGAGGCCCACCCGTGGCCATGCGCTAGGAG |
| | GTGTCTCCCACCCGCCGGAGTCCCAAGGACCCCTCCCAAGAGCTCGGGCACCCTGCGGCCATCA |
| | CACCCAACAGGCGAGTCGGGGTGTAGGAAGTCCACTGCTCACAAGGGCACCCCCTCATTAAACA |
| | TCAGAAATTGGAGACACCCCGGATGGATGGGGGCCTTGGCCCCAAACCCTTTTTCTGTCCCACC |
| | TGTTTCCGTGCCCCTACACCTCCTGTGGGTCTTTTCTTGTCTGTGA |
| 283 | Translation of ORF number 133 in reading frame 1 on the direct |
| | strand |
| | AVSAAGGGRGQWGGALELGRQGRAARGGESLSSPSARAHGGAGEPRAGRCGWGLGAAGRLRVPL |
| | GLGRPALRAPCPWLCPPWDPTSALRKAVGFPCAEAHPWPCARRCLPPAGVPRTPPKSSGTLRPS |
| | HPTGESGCRKSTAHKGTPSLNIRNWRHPGWMGALAPNPFSVPPVSVPLHLLWVESCL |
| 284 | ORF number 134 in reading frame 1 on the direct strand extends |
| | from base 159424 to base 159651 |
| | CCTCAGCCTGGGCGCCTCCAGTTCGGGCTTTCTGCCTACTCAGCAACTTCTAATTTGGGTGCGT |
| | GGTTGGGAGATGCTCTCAGCTGTCAGTCCTGCCCTTGGGGGGCCAGCTTCCTGCCTCTCACAGC |
| | CATTAAGTGCAGCTGGACGCAGGACCCCTGTCCCACTCCTGGGCTGCAGGAGCCACAGGTGAGC |
| | GGTCGGCCGTTGTTCGGCTGCTACCCTGATGCCTGA |
| 285 | Translation of ORF number 134 in reading frame 1 on the direct |
| | strand |
| | PQPGRLQFGLSAYSATSNLGAWLGDALSCQSCPWGASFLPLTAIKCSWTQDPCPTPGLQEPQVS |
| | GRPLFGCYPDA |
| 286 | ORF number 135 in reading frame 1 on the direct strand extends |
| | from base 159919 to base 160251 |
| | GCGGGGCTGACTCCCCGCCCAGCCCTAATCCTGACACAAGCTTTTCTGCCTGTTGCACCGAGGG |
| | GGACCCTCGTCCTCGGACCTGAGGGCACAAGAGGTGCAGGGAGGGGCTCGTGGTGCACATACTG |
| | CGTCCCAGGAGGGGTGGGGGTCCCTAAGCAGTGTCCTCGCGCAGGACTCCTACCGGAAGCAAGT |
| | GGTCATCGATGGGGAGACGTGTCTGCTGGACATCCTGGACACGGCGGGCCAGGAGGAGTACAGC |
| | GCCATGCGGGACCAGTACATGCGCACCGGCGAGGGTTTCCTCTGCGTGTTTGCCATCAACAACA |
| | CCAAGTCCTTTGA |
| 287 | Translation of ORF number 135 in reading frame 1 on the direct |
| | strand |
| | AGLTPRPALILTQAFLPVAPRGTLVLGPEGTRGAGRGSWCTYCVPGGVGVPKQCPRAGLLPEAS |
| | GHRWGDVSAGHPGHGGPGGVQRHAGPVHAHRRGFPLRVCHQQHQVL |
| 288 | ORF number 136 in reading frame 1 on the direct strand extends |
| | from base 160252 to base 160539 |
| | AGACATCCACCAGTACCGGTGAGCTGCCAGCACCCGCGCAGGCCGTCCCTTCTGGCGCCCTGGA |
| | CGCAGCCTGCCGGTGGCTCACACCATCCTCCTTGCAGGGAGCAGATCAAGCGGGTGAAGGACTC |
| | GGACGACGTGCCCATGGTGCTGGTGGGAAACAAGTGTGACCTGGCTGCACGCACTGTGGAGTCT |
| | CGGCAGGCACAGGACCTGGCCCGCAGCTACGGCATCCCCTACATCGAGACCTCGGCCAAGACGC |
| | GCCAGGTGAGCTGGCTCCCTGCCCACCTGTAG |
| 289 | Translation of ORF number 136 in reading frame 1 on the direct |
| | strand |
| | RHPPVPVSCQHPRRPSLLAPWTQPAGGSHHPPCREQIKRVKDSDDVPMVLVGNKCDLAARTVES |
| | RQAQDLARSYGIPYIETSAKTRQVSWLPAHL |
| 290 | ORF number 137 in reading frame 1 on the direct strand extends |
| | from base 160720 to base 161094 |
| | gtcaatttacaaaaaataaaaaagggggagttgtatcccctgacgccccataattacctgtctc |
| | attctctctttattcaaaattttttgaccttggatgcccatggtaagagtgctgcagagcgctt |
| | ttggcatccttctactgcccctcaggctttggtcaaatggaaagacccatttacaggctcttgg |
| | caaggcccagatctagtcctcatatggggccgagggcatgtttgtgtttttccacaggatgcag |
| | aaggccctcggtggctgccagaacgattggtgcgacatgtggaccctctacctgctgatgacat |
| | tgattactctcagcaatgtagaagaagaccagacatattgggcctatgttcctga |
| 291 | Translation of ORF number 137 in reading frame 1 on the direct |
| | strand |
| | VNLQKIKKGELYPLTPHNYLSHSLFIQNFLTLDAHGKSAAERFWHPSTAPQALVKWKDPFTGSW |
| | QGPDLVLIWGRGHVCVFPQDAEGPRWLPERLVRHVDPLPADDIDYSQQCRRRPDILGLCS |
| 292 | ORF number 138 in reading frame 1 on the direct strand extends |
| | from base 163255 to base 163488 |
| | GGCGTGAGTGTCATTGACATAGTCTGGAATCTCAGGaccttcccatacagcagggtggagaata |
| | ggtggatcaggtacgtaggcccaatacgtctggtcttcttctgcattgctgagggtcatcaatg |
| | tcatcagcaggtagagggtccacatgtcgcaccaatcgttctggcagccaccgagggccttctg |
| | tatcctgtggaaaaacacaaacatgccctcggccccatatga |
| 293 | Translation of ORF number 138 in reading frame 1 on the direct |
| | strand |
| | GVSVIDIVWNLRTFPYSRVENRWIRYVGPIRLVFFCIAEGHQCHQQVEGPHVAPIVLAATEGLL |
| | YPVEKHKHALGPI |
| 294 | ORF number 139 in reading frame 1 on the direct strand extends |
| | from base 163810 to base 164130 |
| | ccggagccattatctgttttaagttttttaggagtggcagaagggtgtggtaacccscadmtgg |
| | tcaaatggaaagacccacttacgggctcttggcaaggcccagatccagtcctcatatggggccg |
| | agggcatgtttgtgtttttccacaggatacagaaggccctcggtggctgccagaacgattggtg |
| | cgacatgtggaccctctacttgctgatgacattgatgaccctcagcaatacagaagaagaccag |
| | acgtattscadmcaagcaGATACATTAACAGATTTTTTAGACCAGTCTCTAGTCCCATCTTGTA |
| | A |
| 295 | Translation of ORF number 139 in reading frame 1 on the direct |
| | strand |
| | PEPLSVLSFLGVAEGCGNPXXVKWKDPLTGSWQGPDPVLIWGRGHVCVFPQDTEGPRWLPERLV |
| | RHVDPLLADDIDDPQQYRRRPDVXXXSRYINRFFRPVSSPIL |
| 296 | ORF number 140 in reading frame 1 on the direct strand extends |
| | from base 164356 to base 164601 |
| | agggtccacatgtcgcaccaatcattctggcagccaccgagggccttctgcatcctgtggaaaa |
| | acacaaacatgccctcggccccatatgaggactggatctgggccttgccaagagcctgtaagtg |
| | ggtctttccatttgaccaaagcctgagtggcagcagaaggatgccaaaagcgctccgcagcact |
| | cttaccatgggcatccascadmCTCTAGTCCCGTCTTGTAAATCAGTCACCTGA |
| 297 | Translation of ORF number 140 in reading frame 1 on the direct |
| | strand |
| | RVHMSHQSFWQPPRAFCILWKNTNMPSAPYEDWIWALPRACKWVFPFDQSLSGSRRMPKALRST |
| | LTMGIXXXLVPSCKSVT |
| 298 | ORF number 141 in reading frame 1 on the direct strand extends |
| | from base 164788 to base 165093 |
| | gggtcatcaatgtcatcagcaggtagagggtccacatgtcacaccaatcgttctggcagccacc |
| | gagggccttctgtatcctgtggaaaaacacaaacatgccctcggccccatatgaggactggata |
| | tgggcscadmatttgtggccagcttaattcaagaaagccgtttggaagctcgaaaatattatgg |
| | gaaagagccagatttgattgttgttccttttacaaaaacacagattcaaggcttgatgcagttt |
| | acagacagttttcccatcgccttggctcattttgcaggaactttagataa |
| 299 | Translation of ORF number 141 in reading frame 1 on the direct |
| | strand |
| | GSSMSSAGRGSTCHTNRSGSHRGPSVSCGKTQTCPRPHMRTGYGXXICGQLNSRKPFGSSKILW |
| | ERARFDCCSFYKNTDSRLDAVYRQFSHRLGSFCRNER |
| 300 | ORF number 142 in reading frame 1 on the direct strand extends |
| | from base 165112 to base 166104 |
| | attgcttcagtttttcaacatcatgatccaatttttccttcaattgtgtcacatgctcctcttc |
| | ctgcggtaccaaatgtctttactgatggatctaacaatggtgtcgctgtttatgcactcaataa |
| | acaaattaaaaagatccagacacctccagcttcagctcaaatagttgagcttcgagcagttcat |
| | atggtgttgcttgattttgcttcccagtcttttaatttattctctgacagccattatgtggttc |
| | gtgcagtcaaaaatttagaaacagtaccgtttattaataccagtaatcctgttattcaggattt |
| | atttcttcagatacaacaagccattcagctgcgctgtaaaaaattttatattggccatattaga |
| | gctcactctagtcttccaggccctttagcagcaggcaatcaaattgcagattctgccacgcagc |
| | ttattgccttaactcaaatagaaaaagcacaaaaggctcatagcctccaccatcaaaacagcca |
| | gagcctaagattacagtataagatccccagagaagcagcacgccagattgtaaagcaatgtcct |
| | gactgttcacatttacagcctgtgcctcattatggagttaaccctcggggcttgcgtcccaatg |
| | atctgtggcagacggatgtgactcatatacctgaatttgggaaattaaaatacgtccatgtctc |
| | tatagacacgttctctggctttgtaattacttctggtcaatcaggagaagctacgtctcatgtt |
| | atcagacactgtcttgctgcttttgccatgattggcactcctaaaaaacttaaaacagataatg |
| | gctccggctacaccagcaagaaatttgctttattttgccagcaattttcaattaatcatgttac |
| | tggcattccttacaatccccaaggacaagggattgttgaacgcactcatggcacattaaaagtc |
| | attttacaaaaaataaaaaagggggagttatag |
| 301 | Translation of ORF number 142 in reading frame 1 on the direct |
| | strand |
| | IASVFQHHDPIFPSIVSHAPLPAVPNVFTDGSNNGVAVYALNKQIKKIQTPPASAQIVELRAVH |
| | MVLLDFASQSFNLFSDSHYVVRAVKNLETVPFINTSNPVIQDLFLQIQQAIQLRCKKFYIGHIR |
| | AHSSLPGPLAAGNQIADSATQLIALTQIEKAQKAHSLHHQNSQSLRLQYKIPREAARQIVKQCP |
| | DCSHLQPVPHYGVNPRGLRPNDLWQTDVTHIPEFGKLKYVHVSIDTFSGFVITSGQSGEATSHV |
| | IRHCLAAFAMIGTPKKLKTDNGSGYTSKKFALFCQQFSINHVTGIPYNPQGQGIVERTHGTLKV |
| | ILQKIKKGEL |
| 302 | ORF number 143 in reading frame 1 on the direct strand extends |
| | from base 166105 to base 166485 |
| | cccctgacgccccataattacctgtctcattctctctttattcaacattttttgaccttggatg |
| | cccatggtaagagtgctgcagagcgcttttggcatccttctactgccactcaggctttggtcaa |
| | atggaaagactcacttacaggctcttggcaaggcccagatccagtcctcatatggggccgaggg |
| | catgtttgtgtttttccacaggatgcagaaggccctcggtggctgccagaacgattggtgcgac |
| | atgtggaccctctatttgctgatgascadmGCCATGCACTGTGTCCGCGTCCCGCTCGCTACCA |
| | TTGGGAACCAGCAGCAGCCGCTGCAGCTCTCGCCCCTGAAGGGGCTCAGCCTAGCGGATAA |
| 303 | Translation of ORF number 143 in reading frame 1 on the direct |
| | strand |
| | PLTPHNYLSHSLFIQHFLTLDAHGKSAAERFWHPSTATQALVKWKDSLTGSWQGPDPVLIWGRG |
| | HVCVFPQDAEGPRWLPERLVRHVDPLFADXXXHALCPRPARYHWEPAAAAAALAPEGAQPSG |
| 304 | ORF number 144 in reading frame 1 on the direct strand extends |
| | from base 168031 to base 168300 |
| | TGCAACCAATGTCCAGTGACCCAGATTGCGCTGAACTTTGATGTGTTTACCACTAGGTGGAGCG |
| | GTTTAGCCAAGAAGTTCAGATTACAGAAGCCCGCTGTTTCTATGGCTTCCAAATTGCCATGGAA |
| | AACATACATTCTGAGATGTATAGTCTCCTCATTGACACTTACATCAAAGATTCCAAGGAAAGGT |
| | GAGTATTTGAGTGGTATGCCAACATGTTTGGGACTCACTAATTGTTTATTTCAAGTTTTTGGAT |
| | TCAGACCGGGATAG |
| 305 | Translation of ORF number 144 in reading frame 1 on the direct |
| | strand |
| | CNQCPVTQIALNFDVFTTRWSGLAKKFRLQKPAVSMASKLPWKTYILRCIVSSLTLTSKIPRKG |
| | EYLSGMPTCLGLTNCLFQVFGFRPG |
| 306 | ORF number 145 in reading frame 1 on the direct strand extends |
| | from base 172837 to base 173121 |
| | GCACGCTCGGGCCGGGTTGGGGTGGCGGGTACCTGGGGGACTCGGGCATGCCTCTCACCGCATG |
| | TCTCCCCGCAGCCACCCGCTCTCAACGGCACCCGCGTGCTGGCCAGCAAGGCGGCCCGGAGGAT |
| | CTTCCAGGAGGCGGCGGAGTCCGTGGAGCCGGTGAGCGGATGCCCGAGGGCGGAGACAGCGCAG |
| | TGGGCGTGGCCAGCGCGCAGCGCCTGGGGGCGACAGCCGACTTCGCCGGCTCTCTGGCGCCATG |
| | GCTTTCTTTGTCTTTCTACTTACTCATAA |
| 307 | Translation of ORF number 145 in reading frame 1 on the direct |
| | strand |
| | ARSGRVGVAGTWGTRACLSPHVSPQPPALNGTRVLASKAARRIFQEAAESVEPVSGCPRAETAQ |
| | WAWPARSAWGRQPTSPALWRHGFLCLSTYS |
| 308 | ORF number 146 in reading frame 1 on the direct strand extends |
| | from base 173212 to base 173502 |
| | CAGCTGACACGTAAGACACtggaccacatgaaattgccgacaattgaatgtaactggatgggaa |
| | aaatggcaatttcatatggttcgaTGGATACTTCACATTTTCATTACTTTCTCCCCCAACAGAA |
| | AACTAAGGTGTCTGCCCTCAGCGGGCAGGATGAACCACTGCTGAGAGAAAACCCCCGCCGCTTT |
| | GTCGTCTTTCCCATCGAATACCATGATATCTGGCAGATGTATAAGAAAGCGGAGGCTTCCTTTT |
| | GGACAGCTGAGGAGGTAATCAGATTCAGGAGCTAG |
| 309 | Translation of ORF number 146 in reading frame 1 on the direct |
| | strand |
| | QLTRKTLDHMKLPTIECNWMGKMAISYGSMDTSHFHYFLPQQKTKVSALSGQDEPLLRENPRRF |
| | VVFPIEYHDIWQMYKKAEASFWTAEEVIRFRS |
| 310 | ORF number 147 in reading frame 1 on the direct strand extends |
| | from base 178783 to base 179067 |
| | GCACGCTCGGGCCGGGTTGGGGTGGCGGGTACCTGGGGGACTCGGGCATGCCTCTCACCGCATG |
| | TCTCCCCGCAGCCACCCGCTCTCAACGGCACCCGCGTGCTGGCCAGCAAGGCGGCCCGGAGGAT |
| | CTTCCAGGAGGCGGCGGAGTCCGTGGAGCCGGTGAGCGGATGCCCGAGGGCGGAGACAGCGCAG |
| | TGGGCGTGGCCAGCGCGCAGCGCCTGGGGGCGACAGCCGACTTCGCCGGCTCTCTGGCGCCATG |
| | GCTTTCTTTGTCTTTCTACTTACTCATAA |
| 311 | Translation of ORF number 147 in reading frame 1 on the direct |
| | strand |
| | ARSGRVGVAGTWGTRACLSPHVSPQPPALNGTRVLASKAARRIFQEAAESVEPVSGCPRAETAQ |
| | WAWPARSAWGRQPTSPALWRHGFLCLSTYS |
| 312 | ORF number 148 in reading frame 1 on the direct strand extends |
| | from base 179158 to base 179448 |
| | CAGCTGACACGTAAGACACtggaccacatgaaattgccgacaattgaatgtaactggatgggaa |
| | aaatggcaatttcatatggttcgaTGGATACTTCACATTTTCATTACTTTCTCCCCCAACAGAA |
| | AACTAAGGTGTCTGCCCTCAGCGGGCAGGATGAACCACTGCTGAGAGAAAACCCCCGCCGCTTT |
| | GTCGTCTTTCCCATCGAATACCATGATATCTGGCAGATGTATAAGAAAGCGGAGGCTTCCTTTT |
| | GGACAGCTGAGGAGGTAATCAGATTCAGGAGCTAG |
| 313 | Translation of ORF number 148 in reading frame 1 on the direct |
| | strand |
| | QLTRKTLDHMKLPTIECNWMGKMAISYGSMDTSHFHYFLPQQKTKVSALSGQDEPLLRENPRRF |
| | VVFPIEYHDIWQMYKKAEASFWTAEEVIRFRS |
| 314 | ORF number 149 in reading frame 1 on the direct strand extends |
| | from base 186598 to base 186852 |
| | ctttggatgcccatggtaaaagtgcagctgaacgtttttggcatccttcaactagccctcaggc |
| | cttggtcaaatggaaggacccacttacgggtgtctggcaaggcccagatccagtcctcatatgg |
| | gggcgagggcatgtttgtgtttttccacaggatgcagaaggccctcggtggctgccagaacgat |
| | tggtgcgacatgtggaccctctacctgctgatgacattgatgascadmCTCCGCTTCAGCTAG |
| 315 | Translation of ORF number 149 in reading frame 1 on the direct |
| | strand |
| | LWMPMVKVQLNVFGILQLALRPWSNGRTHLRVSGKAQIQSSYGGEGMFVFFHRMQKALGGCQND |
| | WCDMWTLYLLMTLMXXLRFS |
| 316 | ORF number 150 in reading frame 1 on the direct strand extends |
| | from base 187354 to base 187623 |
| | gacagggagctgatgaatcttttcaagattttgtgtctcgccttactgttgctgcgggacggac |
| | ctttggagcgtccgtggctacggaggctttcattaaacagcttgcttatgaaaatgcaaattct |
| | gcctgccaagcgattattaggcccattaagaaaaaaggcactatctctgattttatccgttcct |
| | gtgccgatgtcggcccctccttttcacagggagtggccctggctgccgctttacaaggaaaaag |
| | cattcatgaagtaa |
| 317 | Translation of ORF number 150 in reading frame 1 on the direct |
| | strand |
| | DRELMNLFKILCLALLLLRDGPLERPWLRRLSLNSLLMKMQILPAKRLLGPLRKKALSLILSVP |
| | VPMSAPPFHREWPWLPLYKEKAFMK |
| 318 | ORF number 151 in reading frame 1 on the direct strand extends |
| | from base 187624 to base 187863 |
| | tgcagcaacaggccaagcttcatgctagtggccgcgcaggagcttgttttaactgtggaaaaat |
| | gggacatcgagcttctcaatgcccacataaaatggaggctaacaatccgtcggctactgctgtg |
| | gttaaaaaacctccagggccttgtcccaggtacaagaaaggcgctcattgggctaataaatgta |
| | aatccaaaactgacaaagacggcaaacccttacagggaaactgggtga |
| 319 | Translation of ORF number 151 in reading frame 1 on the direct |
| | strand |
| | CSNRPSFMLVAAQELVLTVEKWDIELLNAHIKWRLTIRRLLLWLKNLQGLVPGTRKALIGLINV |
| | NPKLTKTANPYRETG |
| 320 | ORF number 152 in reading frame 1 on the direct strand extends |
| | from base 188323 to base 188637 |
| | ttacttgtctttttattcaaaatttttttgactttggatgcctatgttaagagtgcagctgaac |
| | gtttctggcatccttctgccgtccctgaggctttggtcagaaagaaggatccacttactggatc |
| | atggcaaggcccagacccagtcctcatatggggccgagggcatgtttgtgtttttccacaggat |
| | gcagatagtcctcggtggttgccagaacgattggtgcgacatgtggaccctctacctgctgatg |
| | acattgatgaccctcagcaatacagaagaagaccagacgtattgggcctacgtacctga |
| 321 | Translation of ORF number 152 in reading frame 1 on the direct |
| | strand |
| | LLVFLFKIFLTLDAYVKSAAERFWHPSAVPEALVRKKDPLTGSWQGPDPVLIWGRGHVCVFPQD |
| | ADSPRWLPERLVRHVDPLPADDIDDPQQYRRRPDVLGLRT |
| 322 | ORF number 153 in reading frame 1 on the direct strand extends |
| | from base 188725 to base 189525 |
| | tggacacatgaaacaacaTTTGGAAAGTTTTGTAAATCAGGCACTCCCTGCAGTCAGGTGACTG |
| | ATTTACAAGACGGGACTAGAGACTGGTCTAAGAAATCTGTTAATGTATCTGCTTGTGTTCCTTC |
| | CCCTTATACACTTTTGATTGGAAATATTAATGTACATTTTGTAGGAGTTCAGTTTAtggaagat |
| | gtgattcagagtataaaagttaaatcttatttaaaatgtcattcagaatatcattggatatgtg |
| | ttacttcscadmccccggcgacggggcgcgcggggggcggggcggactgtgcccagtgcgcccc |
| | gggcgggtcgcgccgtcgggcccggggggtttccaggcgccacgccgtgaccaaagcacagcga |
| | agcgagcgcacggggtcagcggcgatgtcggccacccacccgacccgtcttgaaacacggacca |
| | aggagtctaacacgtgcgcgagtcaggggctcgcacgaaagccgccgtggcgcaatgaaggtga |
| | aggccggcgccgctcgccggccgaggtgggatcccgaggcctctccagtccgccgagggcgcac |
| | caccggcccgtctcgcccgcagcgccggggaggtggagcacgagcgcacgtgttaggacccgaa |
| | agatggtgaactatgcctgggcagggcgaagccagaggaaactctggtggaggtccgtagcggt |
| | cctgacgtgcaaatcggtcgtccgacctgggtataggggcgaaagactaatcgaaccatctagt |
| | agctggttccctccgaagtttccctcaggatag |
| 323 | Translation of ORF number 153 in reading frame 1 on the direct |
| | strand |
| | WTHETTFGKFCKSGTPCSQVTDLQDGTRDWSKKSVNVSACVPSPYTLLIGNINVHFVGVQFMED |
| | VIQSIKVKSYLKCHSEYHWICVTSXXPATGRAGGGADCAQCAPGGSRRRARGVSRRHAVTKAQR |
| | SERTGSAAMSATHPTRLETRTKESNTCASQGLARKPPWRNEGEGRRRSPAEVGSRGLSSPPRAH |
| | HRPVSPAAPGRWSTSARVRTRKMVNYAWAGRSQRKLWWRSVAVLTCKSVVRPGYRGERLIEPSS |
| | SWFPPKFPSG |
| 324 | ORF number 154 in reading frame 1 on the direct strand extends |
| | from base 189922 to base 190194 |
| | ccttggatgcccatggtaagagtgctgcggagcgcttttggcatccttctgctgccactcaggc |
| | tttggtcaaatggaaagacccacttacaggctcttggcaaggcccagatccagtcctcatatgg |
| | ggccgagggcatgtttgtgtttttccacaggatgcagaaggccctcggtggctgccagaacgat |
| | tggtgcgacatgtggaccctctacctgctgatgacattgatgaccscadmgttgagggtcatca |
| | atgtcatcagcaagtag |
| 325 | Translation of ORF number 154 in reading frame 1 on the direct |
| | strand |
| | PWMPMVRVLRSAFGILLLPLRLWSNGKTHLQALGKAQIQSSYGAEGMFVFFHRMQKALGGCQND |
| | WCDMWTLYLLMTLMTXXLRVINVISK |
| 326 | ORF number 155 in reading frame 1 on the direct strand extends |
| | from base 190195 to base 190644 |
| | agggtccacatgtcgcaccaatcgttctggcagccaccgagggccttctgcatcctgtggaaaa |
| | acacaaacatgccctcggccccatatgaggactggatctgggccttgccaagagcctgtaagtg |
| | ggtctttccatttgaccaaagcctgagtggcagcagaaggatgccaaaagcgctccgcagcact |
| | cttaccatgggcatccaaggtcaaaaaattttgaataaagagagaatgscadmGACCGGGCCGG |
| | GCTCATCGCCCGGCGGCCGCCGCCGCCGCTTTCTCGTtaatgatccttccgcaggttcacctac |
| | ggaaaccttgttacgacttttacttcctctagatagtcaagttcgaccgtcttctcagcgctcc |
| | gccagggccgtgggccgaccccggcggggccgatccgagggcctcactaaaccatccaatcggt |
| | ag |
| 327 | Translation of ORF number 155 in reading frame 1 on the direct |
| | strand |
| | RVHMSHQSFWQPPRAFCILWKNTNMPSAPYEDWIWALPRACKWVFPFDQSLSGSRRMPKALRST |
| | LTMGIQGQKILNKERMXXTGPGSSPGGRRRRFLVNDPSAGSPTETLLRLLLPLDSQVRPSSQRS |
| | ARAVGRPRRGRSEGLTKPSNR |
| 328 | ORF number 156 in reading frame 1 on the direct strand extends |
| | from base 191302 to base 191622 |
| | tcgtcttcgaacctccgactttcgttcttgattaatgaaaacattcttggcaaatgctttcgct |
| | ctggtccgtcttgcgccggtccaagaatttcacctctagcggcgcaatacgaatgcccccggcc |
| | gtccctcttaatcatggcctcagttccgaaaaccaacaaaatagaaccgcggtcctattccats |
| | cadmttgctgagggtcatcaatgtcatcagcaggtagagggtccacatgtcgcaccaatcgttc |
| | tggcagccaccgagggccttctgcatcctgtggaaaaacacaaacatgccctcggccccatatg |
| | a |
| 329 | Translation of ORF number 156 in reading frame 1 on the direct |
| | strand |
| | SSSNLRLSFLINENILGKCFRSGPSCAGPRISPLAAQYECPRPSLLIMASVPKTNKIEPRSYSX |
| | XXAEGHQCHQQVEGPHVAPIVLAATEGLLHPVEKHKHALGPI |
| 330 | ORF number 157 in reading frame 1 on the direct strand extends |
| | from base 191674 to base 191952 |
| | ccaaagcctgagtggcagtggaaggatgccaaaagcgctccgcagcactcttaccascadmtgt |
| | catcagcaggtagagggtccacatgtcgcaccaatcgttctggcagccaccgagggccttctgc |
| | atcctgtggaaaaacacaaacatgccctcggccccatatgaggactgggtctgggccttgccat |
| | gatccagtaagtggatccttctttctgaccaaagcctcagggacggcagaaggatgccagaaac |
| | gttcagctgcactcttaacatag |
| 331 | Translation of ORF number 157 in reading frame 1 on the direct |
| | strand |
| | PKPEWQWKDAKSAPQHSYXXXSSAGRGSTCRTNRSGSHRGPSASCGKTQTCPRPHMRTGSGPCH |
| | DPVSGSFFLTKASGTAEGCQKRSAALLT |
| 332 | ORF number 158 in reading frame 1 on the direct strand extends |
| | from base 192412 to base 192966 |
| | CACTGCCCTTCCTTCGAGCACAGGCTGACCTCAGTGACAGATGAACTGGCTGCGGTCACCGCAG |
| | TGGTGTTCAGCCGGCAGGAGGTGGTCACCCAGCTGCAGCGCGAGCTGCGGAATGAGGAACAGAA |
| | CATCCACCCCCGGCAGCGGTCAGTGGGTCCCACCTATTGTAGCCTTGTGCCCGCGCCCCACCCC |
| | ACACACCTGCCCTGCAGCCAGCTGCAGGCTGAGCCCTCTCTCTGCCCCCTCCCACCTCCCACCT |
| | GCCTGTCTCCTTTCAGGGTTTACCTGCTGGGCAAGAGGCAGGTATTGCAGGAGGAGCTCCAGGG |
| | GCTGCAGGTGGCACTGTGCAGCCAGGCCAAGCTGGAGGCCCAGCAGGATCTTTTGCAGGCCAAG |
| | CTGGAGCAGCTGGGCCCCGGGGATCCCCCGCCTGTGCCGCTCCTACAGGACGACCGCCACTCTA |
| | CCTCCTCCTCGGTGAGTGCCCTACTGCCCTCCGTGGTCACCTTGCTGCCAGCCCAGGCTGTGTC |
| | CTCATTTTCGCCCTCCCCCTCCCCAAGCCTGGCCACCCGCTGA |
| 333 | Translation of ORF number 158 in reading frame 1 on the direct |
| | strand |
| | HCPSFEHRLTSVTDELAAVTAVVFSRQEVVTQLQRELRNEEQNIHPRQRSVGPTYCSLVPAPHP |
| | THLPCSQLQAEPSLCPLPPPTCLSPFRVYLLGKRQVLQEELQGLQVALCSQAKLEAQQDLLQAK |
| | LEQLGPGDPPPVPLLQDDRHSTSSSVSALLPSVVTLLPAQAVSSFSPSPSPSLATR |
| 334 | ORF number 159 in reading frame 1 on the direct strand extends |
| | from base 192967 to base 193197 |
| | CGTCTGTCCCTGGCCTCAGGAGCAGGAGCGGGAAGGGGTACGGACGCCTACCCTGGAGCTCCTG |
| | AAGAGCCACATCTCAGGAATCTTTCGCCCCAAGTTTTCGGTGAGTGGCACCTGTCTGGGCCTGC |
| | GCCTCTGCCCTTCTCCAAGGGGTGGGCTGGGCCAGGGGTCTCAGACATGCCCCCACTGCACCCC |
| | GCCCACATGGTGTTCTGGTTAGCCCCTGGGTTGCCCTAA |
| 335 | Translation of ORF number 159 in reading frame 1 on the direct |
| | strand |
| | RLSLASGAGAGRGTDAYPGAPEEPHLRNLSPQVFGEWHLSGPAPLPFSKGWAGPGVSDMPPLHP |
| | AHMVFWLAPGLP |
| 336 | ORF number 160 in reading frame 1 on the direct strand extends |
| | from base 193198 to base 193455 |
| | AGAGGAGGCTCTCTCCACGCCGCTTTTATTGGGGTGCCAAGCACCAACGTCCCCAGATCCTGCC |
| | ACTCTCACACCCCCTTCTTCTCTGCCATCACATGTGCTGAAGGGACTCACAGCTTTAGTGACCC |
| | CATGGCTCTCCCTGCTCCAGGAGTGGTTGGGGGGCCGCAGCCTGGTGGAAAAGGCAAAAGTTTG |
| | GTTTGGGACCAGTCAGCCGGCCCCCCCATCCCAGCTGTGCCTGGGCCAGTCTATGGCCTGCTCT |
| | AG |
| 337 | Translation of ORF number 160 in reading frame 1 on the direct |
| | strand |
| | RGGSLHAAFIGVPSTNVPRSCHSHTPFFSAITCAEGTHSFSDPMALPAPGVVGGPQPGGKGKSL |
| | VWDQSAGPPIPAVPGPVYGLL |
| 338 | ORF number 161 in reading frame 1 on the direct strand extends |
| | from base 193816 to base 194112 |
| | CGTGAGTGGTGCCAGGACCCGCGCCCACCCTGCCCCACCCTTCCCTGTCACCAGAATGACCTTG |
| | AGAGGGTAGGAAGAAAGGGGCTGCTAGTCTTAGATGCTAGTCAGAGCTGCAAGGGGCCATGGAG |
| | ACCACTTAGTCCCTATAACAGAACAGGCGTAAGTAGCATGGGTAGCAGGTGTGTTGGGCGCCAT |
| | GAGGTCGTGCCTTCCTGCAGTGTCTCTGCCTCTCGTCCCAGGCAGGCCCTTTCTCCCTGCTACT |
| | CTCCCGCTCCCCTCCCAGGGCTCAGGCCCCCTCAGCAGTAG |
| 339 | Translation of ORF number 161 in reading frame 1 on the direct |
| | strand |
| | REWCQDPRPPCPTLPCHQNDLERVGRKGLLVLDASQSCKGPWRPLSPYNRTGVSSMGSRCVGRH |
| | EVVPSCSVSASRPRQALSPCYSPAPLPGLRPPQQ |
| 340 | ORF number 162 in reading frame 1 on the direct strand extends |
| | from base 194113 to base 194427 |
| | AGGCTGCTGACCCCAAGTTGCCCTGCCCTGCAGAACCTGTACCGACTGGAAGGTGATGGTTTTC |
| | CCAGCGTCCCCTTGCTCATTGACCACCTGCTGCAGTCCCAGCAGCCCCTCACCAAGAAGAGCGG |
| | TATTGTCCTGAACAGAGCTGTGCCCAAGGTGAGCCTGCACCCCACCGGCCCACACCACCCACCA |
| | CAGGGTTTGGGGAGCGCGGGTTCAGGCCCACAGAATCGGGGCAGGAGGGGCTTTCCAGGTCTCT |
| | GGTCTACGGTCTGGGTACCACGCGACTCCTCACTCTCCAAGGGGTCAGCTCCCTCCTAG |
| 341 | Translation of ORF number 162 in reading frame 1 on the direct |
| | strand |
| | RLLTPSCPALQNLYRLEGDGFPSVPLLIDHLLQSQQPLTKKSGIVLNRAVPKVSLHPTGPHHPP |
| | QGLGSAGSGPQNRGRRGFPGLWSTVWVPRDSSLSKGSAPS |
| 342 | ORF number 163 in reading frame 1 on the direct strand extends |
| | from base 196108 to base 196377 |
| | GTGCGGGCACGGCCTCGTGCTGCCCACGCCAGCCCCCCAGTAACCCCGCCCAAGCACAGGCCAT |
| | GCTGTCACCCCGTGCCCCCTTTCCCGAGGGACCATGAGTCCTGGGCAGGGAGCGGCCCTTGTTC |
| | ATGTCTATGTGTGGAGTCCCCAGCTCAGGGAGGTGACGGGTGCGGTGTGTGGTGGCTGAGTGAG |
| | CCCCTTTCCTGCTTTATCCAGGGACCTTGCTGCTCGGAACTGCCTGGTCACAGAGAAGAATGTC |
| | TTGAAGATCAGTGA |
| 343 | Translation of ORF number 163 in reading frame 1 on the direct |
| | strand |
| | VRARPRAAHASPPVTPPKHRPCCHPVPPFPRDHESWAGSGPCSCLCVESPAQGGDGCGVWWLSE |
| | PLSCFIQGPCCSELPGHREECLEDQ |
| 344 | ORF number 164 in reading frame 1 on the direct strand extends |
| | from base 196516 to base 196761 |
| | GGCTGGGCGTGCCTCTGGCTGATGGACGTGGGTGGCTCACTCACACTGCCTCACCTCCTTGCAG |
| | GCCGCTATTCGTCCGAGAGCGATGTGTGGAGCTTTGGCATCTTGCTCTGGGAGGCCTTCAGCCT |
| | GGGGGCCTCCCCCTACCCCAACCTCAGCAATCAGCAGACTCGGGAGTTCGTAGAAAAAGGTAAG |
| | GCAACCCCACTGCATGACAGCAGCCCGACCCACGCGCTCATCCCAGTGCTATAG |
| 345 | Translation of ORF number 164 in reading frame 1 on the direct |
| | strand |
| | GWACLWLMDVGGSLTLPHLLAGRYSSESDVWSFGILLWEAFSLGASPYPNLSNQQTREFVEKGK |
| | ATPLHDSSPTHALIPVL |
| 346 | ORF number 165 in reading frame 1 on the direct strand extends |
| | from base 197161 to base 197598 |
| | CGCTGTGTTCAGGCTCATGGAGCAGTGCTGGGCCTACGAGCCCAGTCAGCGACCCAGCTTCAGC |
| | ACCATCTACCAGGAGCTGCAGACCATCCGAAAGCGGCATCGGTGAGGCTCGGCCCGCTTCTCAA |
| | GCCAGTGGCTTCTGTTGGCAAGATTATACCTCCTCCCCAGCTCCAGCTCACACCGTGGGACAGC |
| | CCTTCCCAGTCCTGGACTCTGGCCGCCGGCATCCATGCTGCCAGGGGGGATGCAGCTCCATGTC |
| | TGCTGTGCGTCCCCATTCCTGCCAGscadmgatttaacctttatgctttgaatgacatctccca |
| | TATACTGAACTCCTACAAAATGTACATTAATATTTCCAATCAAAAGTGTATATGGGGAAGGAAC |
| | ACAAGCAGATATATTAACAGATTTCTTAGACCAGTCTCTAGTCCCGTCTGGTAA |
| 347 | Translation of ORF number 165 in reading frame 1 on the direct |
| | strand |
| | RCVQAHGAVLGLRAQSATQLQHHLPGAADHPKAASVRLGPLLKPVASVGKIIPPPQLQLTPWDS |
| | PSQSWTLAAGIHAARGDAAPCLLCVPIPAXXRFNLYALNDISHILNSYKMYINISNQKCIWGRN |
| | TSRYINRFLRPVSSPVW |
| 348 | ORF number 166 in reading frame 1 on the direct strand extends |
| | from base 197797 to base 198024 |
| | gggtcatcaatgtcatcagcaggtagagggtccacatgttgcaccaatcgttctggcagccacc |
| | gaggactatctgcatcctgtggaaaaacacaaacatgccctcggccccatatgaggactgggtc |
| | tgggccttgccatgatccagtaagtggatccttccttctgaccaaagcctcagggacggcagaa |
| | ggatgccagaaacgttcagctgcactcttaacatag |
| 349 | Translation of ORF number 166 in reading frame 1 on the direct |
| | strand |
| | GSSMSSAGRGSTCCTNRSGSHRGLSASCGKTQTCPRPHMRTGSGPCHDPVSGSFLLTKASGTAE |
| | GCQKRSAALLT |

What is claimed is:

1. An induced pluripotent bat stem cell (bat IPSC), wherein the cell is in a pluripotent state.

2. The bat IPSC of claim 1, wherein the cell is in a pluripotent state characterized by the expression of one or more factors selected from the group of Klf4, Klf17, Essrb, Tfcp2l1, Tfe3, Dppa, Oct4, Sox2, Nanog, and Dusp6.

3. The bat IPSC of claim 1 or 2, wherein the cell is in a naïve pluripotent state.

4. The bat IPSC of any one of claims 1-3, wherein the cell further is characterized by the expression of one or more factors selected from the group of Otx2 or Zic2.

5. The bat IPSC of any one of claims 1-4, wherein the cell is derived from a bat fibroblast.

6. The bat IPSC of claim 5, wherein the cell is derived from a bat embryonic fibroblast or a bat fibroblast from an adult bat.

7. The bat IPSC of any one of claims 1-6, wherein the cell is derived from a Rhinolophus bat or a Myotis bat.

8. The bat IPSC of claim 7, wherein the cell is derived from a Rhinolophus ferrumequinum bat or a Myotis myotis bat.

9. The bat IPSC of any one of claims 1-8, wherein the cell is capable of differentiating into embryonic bodies.

10. The bat IPSC of claim 9, wherein the embryonic bodies are capable of differentiating into three-dimensional structures comprising three germ layer markers.

11. A method of producing induced pluripotent bat stem cells (bat IPSCs), the method comprising:
(i) reprogramming isolated bat cells with Oct4, Sox2, cMyc, and Klf4 factors;
(ii) culturing the reprogrammed cells on feeder cells in a medium comprising FGF, Leukemia inhibitory factor (Lif), SCF, and Forskolin until colonies appear; and
(iii) splitting cells using a low concentration EDTA buffer;
thereby producing IPSCs from bats.

12. The IPSCs produced by the method of claim 11.

13. The method of claim 11 or claim 12, wherein the isolated bat cell is a bat fibroblast.

14. The method of claim 13, wherein the isolated bat cell is a bat embryonic fibroblast or an bat adult fibroblast.

15. The method of any one of claims 11-14, wherein the isolated bat cell is derived from a Rhinolophus bat.

16. The method of claim 15, wherein the isolated bat cell is derived from a Rhinolophus ferrumequinum bat.

17. The method of any one of claims 11-16, wherein the Lif is at a concentration of 10∝U/ml.

18. The method of any one of claims 11-17, wherein the FGF is at a concentration of 100 ng/ml.

19. The method of any one of claims 11,-18 wherein the SCF is at a concentration of 100 ng/ml.

20. The method of any one of claims 11-19, wherein the Forskolin is at a concentration of 20 nM.

21. The method of any one of claims 11-20, wherein the feeder cell is a mouse CF1 mouse embryonic fibroblasts (MEF).

22. The method of any one of claims 11-21, the method further comprising passaging the bat IPSCs every 5 days onto feeder cells.

23. The method of any one of claims 11-22, wherein the bat IPSC is further differentiated into embryonic bodies.

24. The method of claim 23 wherein the embryonic bodies are further differentiated into three-dimensional structures comprising three germ layer markers.

25. A method of producing induced pluripotent bat stem cells (bat IPSCs), the method comprising:
(i) reprogramming isolated bat cells with Oct4, Sox2, cMyc, and Klf4 factors;
(ii) culturing the reprogrammed cells in feeder free medium comprising FGF, Leukemia inhibitory factor (Lif), SCF, and Forskolin until colonies appear; and
(iii) splitting cells using a low concentration EDTA buffer
thereby producing IPSCs from bats.

26. A composition for reprogramming a bat cell to produce pluripotent stem cells comprising a medium comprising FGF, Leukemia inhibitory factor (Lif), SCF, and Forskolin.

27. The composition of claim 18, wherein the Lif is at a concentration of 10\hat{ }4 U/ml.

28. The composition of claim 18, wherein the FGF is at a concentration of 100 ng/ml.

29. The composition of claim 18, wherein the SCF is at a concentration of 100 ng/ml.

30. The composition of claim 18, wherein the Forskolin is at a concentration of 20 nM.

31. A method of obtaining viral sequences from bat IPSCs, the method comprising
obtaining bat IPSCs;
identifying viral sequences residing in the bat iPSC genome or intracellular virus genome; and
assembling the viral sequences;
thereby obtaining viral sequences from the bat iPSCs.

32. The method of claim 31, wherein the identifying comprises sequencing the bat genome or the genome of viral particles residing in the bat IPSCs, or of viral particles shed by the bat IPSCs.

33. The method of claim 31 or claim 32, wherein the identifying comprises sequencing the RNA of the bat genome or the genome of viral particles residing in the bat IPSCs, or of viral particles shed by the bat IPSCs.

34. The method of claim 31, wherein the identifying the proteins and peptides produced by the viral genome by proteomics e.g., LC-MS.

35. The method of claim 31, further comprising translating the sequence into a protein sequence and determining whether the translated sequence has a significant homology to a known protein sequence in a viral protein database.

36. The method of claim 35, wherein the sequence is selected from SEQ ID NO: 1-349.

37. The method of claim 31, wherein the virus is selected from the group of a SARS-CoV-2 virus, endogenous retrovirus (RfRV), and sindbis virus.

38. The method of claim 31, wherein the virus is a coronavirus.

39. The method of claim 35, wherein the sequence is encoding a gag protein, a pol protein, or an env Protein.

40. A method of obtaining viral sequences from virus particles shed by bat IPSCs or cells derived from bat IPSCs, the method comprising
obtaining bat IPSCs or cells derived from bat IPSCs;
culturing the bat IPSCs or cells derived from bat IPSCs under conditions that allows shedding of virus particles into the culture media;
collecting the culture media;
identifying viral sequences residing in the culture media; and
assembling the viral sequences,
thereby obtaining viral sequences from virus particles shed by bat iPSCs or cells derived from bat IPSCs.

41. Use of any one of the viral sequences of claims 31-40 for the development of a vaccine.

42. A recombinant nucleic acid molecule, comprising
a promoter, and
a nucleic acid selected from SEQ ID NO: 1-349 encoding for a viral protein or fragment thereof.

43. A recombinant, replication deficient adenovirus, comprising the nucleic acid of claim 42.

44. A mRNA comprising the nucleic acid of claim 42.

45. An expression vector comprising
a promoter and
a nucleic acid selected from SEQ ID NO: 1-349 encoding for a viral protein or fragment thereof.

46. An isolated protein or peptide comprising an amino acid sequence encoded in a nucleic acid set forth in SEQ ID NO: 1-349, wherein the peptide is no more than 100 amino acids in length, and an optional pharmaceutically acceptable carrier.

47. The isolated protein or peptide of claim 46, wherein the protein or peptide is no more than 30 amino acids in length or 20 amino acids in length.

48. The isolated protein or peptide of claims 46 or 47, where the protein or peptide is synthetic.

49. A pharmaceutical composition comprising the adenovirus of claim 43, the mRNA of claim 44, or the protein or peptide of any one of claims 46-48 and a pharmaceutically acceptable carrier or excipient.

50. A pharmaceutical composition comprising a plurality of (e.g., 2, 3, 4, 5, 6, 7, 8, 9, 10, or more) proteins or peptides of any one of claims 46-48 and a pharmaceutically acceptable carrier or excipient.

51. A pharmaceutical composition comprising a nucleic acid encoding the mRNA of claim 44 or the protein or peptide of any one of claims 46-48 and a pharmaceutically acceptable carrier or excipient.

52. A pharmaceutical composition comprising one or more nucleic acids encoding a plurality of (e.g., 2, 3, 4, 5, 6, 7, 8, 9, 10, or more) mRNAs of claim 44 or proteins or peptides of any one of claims 46-48, and a pharmaceutically acceptable carrier or excipient.

53. The pharmaceutical composition of any one of claims 49-52, further comprising a liposome, wherein the protein or peptide or the nucleic acid encoding the protein or peptide is disposed within the liposome.

54. The pharmaceutical composition of any one of claims 49-52, further comprising a lipid nanoparticle, wherein the protein or peptide or the nucleic acid encoding the protein or peptide is disposed within the lipid nanoparticle.

55. The pharmaceutical composition of any one of claims 49-54, further comprising an immunogenicity enhancing adjuvant.

56. The pharmaceutical composition of any one of claims 49-55, wherein the protein or peptide or nucleic acid encoding the protein or peptide is synthetic.

57. A vaccine that stimulates a T cell mediated immune response when administered to a subject, the vaccine comprising the pharmaceutical composition of any one of claims 49-56.

58. A vaccine comprising the pharmaceutical composition of any one of claims 49-57.

59. The vaccine of claims 57 or 58, wherein the vaccine is a priming vaccine and/or a booster vaccine.

60. A recombinant cell comprising a nucleic acid or a portion of a nucleic acid set forth in SEQ ID NO: 1-349.

61. A recombinant cell comprising a protein or a portion of a protein encoded by a nucleic acid set forth in SEQ ID NO: 1-349.

62. A composition comprising an inhibitor of a protein encoded by a nucleic acid selected from SEQ ID NO: 1-349.
